(19) United States
(12) Patent Application Publication
(10) Pub. No.: US 2026/0234583 A1
(43) Pub. Date: Aug. 13, 2026

(54) PERSONALIZED CRISPR PROFILING FOR CANCER

(71) Applicant: Integrate Bioscience LLC, San Diego, CA (US)

(72) Inventors: Stephane CORBEL, San Diego, CA (US); Karl HODEL, Dana Point, CA (US); Christian SCHMEDT, El Cerrito, CA (US); Srihari C. SAMPATH, San Diego, CA (US); Srinath C. SAMPATH, San Diego, CA (US)

(21) Appl. No.: 19/155,227

(22) PCT Filed: Feb. 9, 2024
(86) PCT No.: PCT/US2024/015256

§ 371 Date: Aug. 8, 2025

Related U.S. Application Data
(60) Provisional application No. 63/484,468, filed on Feb. 10, 2023.

(51) Int. Cl. C12N 9/22; A61K 31/713; C12N 15/11; C12N 15/113; C12N 15/86; C40B 40/06

(52) U.S. Cl. CPC C12N 9/22; A61K 31/713; C12N 15/111; C12N 15/113; C12N 15/86; C40B 40/06; C07K 2319/09; C07K 2319/81; C12N 2310/122; C12N 2310/14; C12N 2310/20; C12N 2740/15041

(57) ABSTRACT

Provided are methods for treating cancer in a subject by selecting a therapeutic molecule after modifying cancer cells from the subject by knocking down gene function of one or more genes without targeted DNA cleavage of one or more genomic loci. Also provided herein are methods of perturbing gene function in one or more modified cancer cells from a subject without targeted DNA cleavage of one or more genomic loci. Also provided herein are CRISPRi systems for gene modulation and high titer CRISPRi lentiviral gene modulation solutions for knockdown of target gene function in primary cells.

### CROSS-REFERENCE TO RELATED APPLICATION

This application claims the benefit of U.S. provisional application Ser. No. 63/484,468 filed Feb. 10, 2023; which is hereby incorporated by reference in its entirety.

### SEQUENCE LISTING

The instant application contains a Sequence Listing which has been submitted electronically in XML format and is hereby incorporated by reference in its entirety. Said XML copy, created on Feb. 7, 2024, is named 56322-704_601_SL.xml and is 24,304,018 bytes in size.

### BACKGROUND

Cancers are extraordinarily heterogeneous, differing in DNA sequence, epigenomic landscape, RNA expression, and protein levels, resulting in vast combinatorial complexity in cell behavior. The extreme molecular complexity and heterogeneity underlying cancer cell behavior has led to dramatic shortfalls to predict which patients will benefit from any particular therapy. The lack of an effective means of predicting patient response directly leads to cycles of futile therapy, at enormous opportunity cost to patients and economic cost to both patients and healthcare payers. Therefore, there remains a need to improve profiling methods in order to identify and predict effective cancer therapy in each patient.

### SUMMARY

Provided herein are methods of treating cancer in a subject in need thereof. In one aspect described herein, the methods of treating cancer in a subject in need thereof comprise administering to the subject a therapeutic molecule selected from a compilation of therapeutic molecules; wherein the therapeutic molecule has been selected by a method comprising: modifying cancer cells from the subject by knocking down the function of a plurality of genes without targeted DNA cleavage of one or more genomic loci to generate modified cancer cells, each gene in the plurality of genes encoding for a protein target of a therapeutic molecule in the compilation of therapeutic molecules, whereby the therapeutic molecule has been selected if knocking down the function of the gene that encodes for the protein target of the selected therapeutic molecule impairs cancer cell viability or proliferation rate during in vitro propagation. In some embodiments, modifying cancer cells from the subject by knocking down the function of the plurality of genes comprises knocking down gene transcription of the plurality of genes. In some embodiments, knocking down gene transcription of the plurality of genes comprises (i) reducing mRNA expression of the plurality of genes to below a threshold level, and/or (ii) altering a phenotype of the modified cancer cells. In some embodiments, modifying cancer cells comprises perturbing the function of the plurality of genes without induction of a double-strand break (DSB) response in a majority of the modified cancer cells, wherein a majority comprises greater than 50% of the modified cancer cells. In some embodiments, knocking down the function of the plurality of genes comprises using CRISPR interference (CRISPRi). In some embodiments, using CRISPRi comprises using a catalytically inactive form of a CAS endonuclease. In some embodiments, the catalytically inactive form of the CAS endonuclease is delivered to the cancer cells in a single vector. In some embodiments, the catalytically inactive form of the CAS endonuclease is delivered to the cancer cells in two or more vectors. In some embodiments, the catalytically inactive form of CAS endonuclease comprises dCas9 dCas12a, dCas12e, dCas12f, dCas12f1, dCasMINI, dCas12j2, or dCas12j3. In some embodiments, the catalytically inactive form of CAS endonuclease is fused to an epigenetic modulator. In some embodiments, the epigenetic modulator comprises a KRAB (also referred to as KOX), SID, MBD2, MBD3, HPla, DNMT family (including DNMT1, DNMT3A, DNMT3B, DNMT3L, DNMT2A), Sin3a, Rb, MeCP2, ROM2, AtHD2A, LSD1, SUV39H1, G9a (EHMT2), ZFP1, ZFP14, ZFP82, ZIM2, ZIM3, ZNF18, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF419, ZNF140, ZNF175, ZNF214, ZNF184, ZNF8, ZNF60, ZNF595, ZNF596, ZNF10, ZNF37A, ZNF394, ZNF610, ZNF273, ZNF34, ZNF250, ZNF98, ZNF675, ZNF213, ZFP28-2, ZNF224, ZNF257, GLP (EHMT2), or a variant or combination thereof. In some embodiments, the KRAB, wherein the KRAB comprises a KRAB, or variant thereof, selected from Table 1 or Table 2. In some embodiments, the KRAB, wherein the KRAB comprises a ZNF10 KRAB. In some embodiments, the catalytically inactive form of CAS endonuclease is fused to the epigenetic modulator via a linker. In some embodiments, the catalytically inactive form of CAS endonuclease has one or more protein domains positioned in between the catalytically inactive form of the CAS endonuclease and the epigenetic modulator. In some embodiments, the linker has about one to about 100 amino acids, optionally a flexible linker, and further optionally the linker is about 16 amino acids. In some embodiments, the catalytically inactive form of the CAS endonuclease comprises a nuclear localization sequence (NLS) positioned amino-terminal (N-terminal) to a RuvC-1 domain of the catalytically inactive form of the CAS endonuclease. In some embodiments, the NLS positioned N-terminal to the RuvC-1 domain comprises a triple SV40 NLS. In some embodiments, the catalytically inactive form of the CAS endonuclease comprises an NLS positioned carboxy-terminal (C-terminal) to a RuvC-III domain of the catalytically inactive form of the CAS endonuclease. In some embodiments, the NLS positioned C-terminal to the RuvC-III domain comprises a single nucleoplasmin NLS. In some embodiments, the catalytically inactive form of the CAS endonuclease comprises a peptide-linked downstream reporter C-terminal to a RuvC-III domain of the catalytically inactive form of the CAS endonuclease. In some embodiments, the peptide-linked downstream reporter C-terminal to the RuvC-III domain comprises a self-cleaving T2A-peptide linked downstream reporter. In some embodiments, the self-cleaving T2A-peptide linked downstream reporter is linked to the catalytically inactive form of CAS endonuclease C-terminal relative to the NLS positioned C-terminal to the RuvC-III domain. In some embodiments, the self-cleaving T2A-peptide linked downstream reporter comprises Thyl. In some embodiments, the epigenetic modulator represses transcription of each of the plurality of genes. In some embodiments, the epigenetic modulator inhibits RNA polymerase binding to a promoter sequence near a transcription start site of each of the plurality of genes. In some embodiments, the epigenetic modulator stalls RNA polymerase during mRNA transcription of each of plurality of genes. In some embodiments, the epigenetic modulator inhibits DNA helicase function near a region of genomic DNA for each of the plurality of genes. In some embodiments, using CRISPRi comprises using PAM anchored gene interference without creating double-strand breaks in DNA for each of the plurality of genes. In some embodiments, using CRISPRi comprises using a library of gene modulatory reagents to generate the modified cancer cells. In some embodiments, the library of gene modulatory reagents comprises a nucleic acid sequence at least 80% identical to at least about 15 contiguous nucleotides of a target gene of Table 3, Table 4, Table 9A, Table 9B, Table 9C, Table 9D, Table 10A, Table 10B, or Table 10C. In some embodiments, one or more of the library of gene modulatory reagents comprise a guide RNA (gRNA) sequence comprising complementarity to at least a portion of the gene that encodes a protein target of a therapeutic molecule in the compilation of therapeutic molecules. In some embodiments, the gRNA sequence further comprises a barcode sequence. In some embodiments, the barcode sequence comprises an iBAR barcode sequence incorporated into a first stemloop of the gRNA sequence. In some embodiments, the library comprises a plurality of random sequence labels (RSLs) incorporated during library amplification. In some embodiments, the plurality of random sequence labels comprises a plurality of random sequence barcodes of about 1 to about 15 nucleotides each. In some embodiments, the complementarity to at least a portion of the gene comprises complementarity to the sense strand of each of the plurality of genes. In some embodiments, the complementarity to at least a portion of the gene comprises complementarity to the antisense strand of each of the plurality of genes. In some embodiments, the at least a portion of the gene is downstream of a transcriptional start site for each of the plurality of genes. In some embodiments, a majority of the modified cancer cells do not exhibit a mild non-specific fitness defect. In some embodiments, the knocking down the function of the plurality of genes comprises using Transcription activator-like effector (TALE) repression. In some embodiments, using TALE repression comprises using an engineered TALE binding protein fused to an epigenetic modulator. In some embodiments, the epigenetic modulator comprises an epigenetic modulator domain, or variant thereof, which is listed in Table 1 or Table 2. In some embodiments, knocking down the function of the plurality of genes comprises using a catalytically inactive zinc finger nuclease. In some embodiments, modifying cancer cells comprises post-transcriptional knocking down of gene mRNA function. In some embodiments, post-transcriptional knocking down of gene mRNA function comprises using RNA interference (RNAi). In some embodiments, using RNAi comprises introducing a plurality of double-stranded RNA (dsRNA) molecules comprising RNA sequence complimentary to mRNA sequences of the plurality of genes into the cancer cells to generate the modified cancer cells. In some embodiments, using RNAi comprises introducing a plurality of small hairpin RNA (shRNA) molecules comprising RNA sequence complimentary to mRNA sequences of the plurality of genes into the cancer cells to generate the modified cancer cells. In some embodiments, components used for knocking down the function of the plurality of genes are delivered by a viral vector. In some embodiments, the viral vector comprises a retroviral vector, an adenoviral vector, an adeno-associated viral vector, or a herpes simplex viral vector. In some embodiments, the retroviral vector comprises a lentiviral vector. In some embodiments, the lentiviral vector comprises a pseudotyped lentiviral vector. In some embodiments, the pseudotyped lentiviral vector comprises a plurality of envelope glycoproteins, wherein the plurality of envelope glycoproteins are derived from Morbillivirus, Sendai virus (SeV), Nipah virus (NiV), Newcastle disease virus (NDV), or Baboon endogenous virus (BaEV), or a combination thereof; and/or wherein the plurality of envelope glycoproteins are attached to an antibody or antibody fragment thereof, including, but not limited to, a single-chain antibody fragment, nanobody, or darpin. In some embodiments, modifying cancer cells comprises depleting a target protein level during in vitro propagation of the modified cancer cells by at least 10%, 20%, 30%, 40%, 50%, 60%, 70%, 75%, 80%, 85%, 90%, 95%, or 99% compared to a target protein level prior to modifying the cancer cells. In some embodiments, the compilation of therapeutic molecules comprises at least 1, at least 2, at least 3, at least 4, at least 5, at least 10, or at least 20 therapeutic agents of Table 3, Table 4, Table 10A, Table 10B, and/or Table 10C. In some embodiments, the selected therapeutic molecule comprises sorafenib, gilteritinib, quizartinib, glasdegib, ivosidenib, midostaurin, venetoclax, pemigatinib, or ruxolitinib. In some embodiments, the cancer cells prior to modification exhibit an active DNA damage response. In some embodiments, the cancer cells prior to modification exhibit a normal or a near-normal p53-dependent DNA double strand break (DSB) response. In some embodiments, the normal or the near-normal p53-dependent DNA DSB response in the cancer cells prior to modification reflects an extent of p53-dependent DNA DSB response of the cancer of the subject. In some embodiments, the cancer in the subject comprises a hematologic malignancy. In some embodiments, the hematologic malignancy comprises a leukemia, a lymphoma, a myeloma, or a combination thereof. In some embodiments, the leukemia comprises Acute Myeloid Leukemia (AML). In some embodiments, the cancer in the subject comprises Glioblastoma Multiforme, Soft Tissue Tumors and Sarcomas, Kidney Adenomas and Adenocarcinomas, Liver Hepatocellular Carcinoma, Adrenocortical Carcinoma, Skin Cutaneous Melanoma, Mesothelioma, Prostate Adenocarcinomas, Diffuse Large B-cell Lymphoma, Cholangiocarcinoma, Cervical Squamous Cell Carcinoma and Endocervical Adenocarcinoma, Acute Myeloid Leukemia, Multiple Myeloma, Thymoma, Kidney Renal Clear Cell Carcinoma, Kidney Renal Papillary Cell Carcinoma, Acute Lymphoblastic Leukemia, Testicular Germ Cell Tumors, or Thyroid Carcinoma. In some embodiments, the cancer cells from the subject comprise primary cancer cells. In some embodiments, the cancer cells from the subject are isolated ex vivo and assayed for cancer cell viability or proliferation rate during in vitro propagation. In some embodiments, the cancer cells prior to modification do not comprise a mutation in p53. In some embodiments, the impairment of cancer cell viability or proliferation rate during in vitro propagation indicates a determination of a functional response of the cancer of the subject. In some embodiments, the determination further comprises a functional call for suggested therapy. In some embodiments, the functional call for suggested therapy indicates a weak, an intermediate, or a strong predicted response to the selected therapeutic molecule. In some embodiments, the methods further comprise a weak predicted response to one or more small molecule therapeutics drugs listed in Table 3, 4, 10A, 10B, or 10C, an intermediate predicted response to one or more small molecule therapeutics drugs listed in Table 3, 4, 10A, 10B, or 10C, or a strong predicted response to one or more small molecule therapeutics drugs listed in Table 3, 4, 10A, 10B, or 10C.

In another aspect described herein, are methods of treating cancer in a subject in need thereof comprise administering to the subject a therapeutic molecule selected from a compilation of therapeutic molecules; wherein the cancer in the subject has been determined to be susceptible to the selected therapeutic molecule by a method comprising: contacting a sample of cancer cells from the subject with a library of gene modulatory reagents to generate a plurality of modified cancer cells, wherein each modified cancer cell harbors one or more of the gene modulatory reagents, wherein the one or more of the gene modulatory reagents are capable of knocking down the function of a gene that encodes a protein target of a therapeutic molecule in the library of therapeutic molecules, wherein the one or more of the gene modulatory reagents does not cleave DNA, and sequencing a marker from each of the one or more of the gene modulatory reagents in the plurality of modified cancer cells, wherein a gene modulatory reagent that impairs cell viability or proliferation rate will have fewer sequence reads than a gene modulatory reagent that does not impair cell viability or proliferation rate, wherein the one or more of the gene modulatory reagents capable of knocking down the function of the gene comprise a sequence complementarity to a gene that encodes for the protein targeted by the selected therapeutic molecule. In some embodiments, the sequencing comprises sequencing DNA extracted from the plurality of modified cancer cells. In some embodiments, the marker from each of the one or more of the gene modulatory reagents comprises a barcode sequence. In some embodiments, the marker from each of the one or more of the gene modulatory reagents comprises a random sequence label. In some embodiments, the marker from each of the one or more of the gene modulatory reagents comprises a nucleotide sequence encoding an sgRNA.

Provided herein are methods of perturbing gene function in a plurality of modified cancer cells from a subject having cancer. In an aspect described herein, the methods of perturbing gene function in a plurality of modified cancer cells from a subject having cancer comprise delivering a library of gene modulatory reagents to a sample of cancer cells from the subject to generate the plurality of modified cancer cells; wherein each modified cancer cell harbors one or more of the gene modulatory reagents, and each gene modulatory reagent is capable of perturbing the function of a gene that encodes a protein target from a compilation of protein targets.

Provided herein are compilations comprising a plurality of modified cancer cells. In an aspect described herein, compilations comprise a plurality of modified cancer cells, wherein each modified cancer cell harbors one or more gene modulatory reagents, and each gene modulatory reagent is capable of knocking down the function of a gene that encodes a protein target from a compilation of protein targets.

Provided herein are libraries comprising a plurality of gene perturbation reagents. In an aspect described herein, libraries comprise a plurality of gene perturbation reagents, wherein each gene perturbation reagent is capable of knocking down the function of a gene that encodes a protein target from a library of protein targets.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a graphical representation of an example Cas9/sgRNA plasmid used for expression of the Cas9 enzyme and an sgRNA specific for a target gene.

FIG. 2 shows a graph of scatter plots for gene dependency calling using CRISPRi gene modulation in the Kasumi-1 AML cell line.

FIG. 3A-FIG. 3B show a successful establishment of a CRISPR-based genetic screen using cells from AML patients as quantified by the transduction efficiency and cell numbers at the end of the CRISPR-based screen. As shown in FIG. 3A, cell surface expression of mThyl was quantified by flow cytometry to access transduction efficiency in the cell population. Transduction above 5% was considered as successful. Control samples without virus were used as negative control for mThyl expression. FIG. 3B shows the cell counting of 63 individual AML samples collected at the end of the CRISPR-screen experiment (Day 8). These cells were able to survival and proliferate. The dotted line indicates the starting number of cells.

FIG. 4A-FIG. 4F shows scatter plots of 6 representative CRISPRi screens on primary AML samples. The results show that CRISPR-based screening allows identification of FLT3, RET (b-Raf pathway) and BIRC5 as essential genes for AML survival. FIG. 4A and FIG. 4B demonstrate FLT3 dependency. FIG. 4C demonstrates FLT3 and RET dependency. FIG. 4D and FIG. 4F demonstrate FLT3 and BIRC5 dependency. FIG. 4E demonstrates BIRC5 dependency.

FIG. 5A-FIG. 5B shows scatter plots of 2 CRISPRi screens on primary AML samples. The results show that CRISPR-based screening allows for an retrospective functional genomic prediction of response to sorafenib therapy by identifying known therapeutic targets of sorafenib (FLT3, ABL1, BRAF) as being functionally dependent in AML cells derived from a patient (FX166) determined to be a sorafenib responder during treatment in FIG. 5A and no identification of known therapeutic targets of sorafenib as being functionally dependent in AML cells derived from a patient (FX165) determined to be a sorafenib non-responder during treatment in FIG. 5B.

FIG. 6A-FIG. 6B shows scatter plots of 8 CRISPRi screens on primary AML samples from subjects established to be clinically responsive to sorafenib therapy to determine functional genomic dependence on genes tested including known therapeutic targets of sorafenib. A functional genomic prediction of sorafenib responsiveness was determined for each sample. Results from responding subjects FX166, FX167, FX175, and FX176 are graphed in FIG. 6A. Results from responding subjects FX211, FX220, FX221, and FX257 are graphed in FIG. 6B.

FIG. 7 shows scatter plots of 6 CRISPRi screens on primary AML samples from subjects established to be clinically responsive to sorafenib therapy to determine functional genomic dependence on genes tested including known therapeutic targets of sorafenib. A functional genomic prediction of sorafenib responsiveness was determined for each sample.

FIG. 8 shows scatter plots of 8 CRISPRi screens on primary AML samples from subjects established to be clinically non-responsive to sorafenib therapy to determine functional genomic dependence on genes tested including known therapeutic targets of sorafenib. A functional genomic prediction of sorafenib responsiveness was determined for each sample.

### DETAILED DESCRIPTION OF THE INVENTION

Cancers are heterogenous and often have a variety of molecular changes and backgrounds. It is difficult for healthcare providers to identify a specific drug or drug combination for each cancer patient, and various drug regimens often require ongoing modification. The present disclosure provides personalized means of treating cancer based on genetic screening using primary cancer cells obtained from patients to identify effective target(s) for cancer treatment. Certain methods described herein comprise, establishing cell cultures obtained from clinical samples of cancer patients; transducing cancer cell cultures with gene modification systems to alter function of a plurality of genes, wherein each gene in the plurality of genes encodes a protein target of a therapeutic molecule in the compilation of therapeutic molecules; maintaining the cell cultures for a period of time; collecting cells to assess cell viability or proliferation; identifying target hits; and selecting the therapeutic molecules corresponding to the target hits for treatment in each cancer patient. The methods as described herein provide new treatment opportunities that are tailored to be more effective and specific for individual cancer patients.

AML is the most common acute leukemia in adults over 50 years of age. Current therapeutic approaches often focus on high or low dose chemotherapy in a front line setting, with use of molecularly targeted agents in unfit patients or in a relapsed or refractory setting. Genomic profiling for common molecular lesions associated with AML (e.g. FLT3) has been used to stratify the target patient population, however this is often an incomplete stratification and positive identification of oncogenic molecular lesions in cells from a subject with AML does not always corresponding to a functional deficiency within molecular pathway of the genetic lesion. This genetic mutation-based approach to determining targeted cancer therapeutics has proven to only be moderately successful. A more personalized and comprehensive approach focused on determining functional gene dependencies for a given sample and then selecting specific therapeutics known to target the molecular pathways of these functional gene dependencies could be of significant clinical benefit. This approach could lead to more favorable patient-based outcomes, more favorable patient quality of life, and/or to a lower probability of therapeutic switching during a period of treatment. The solution presented herein was the development of an in vitro functional genomics platform for the systemic characterization of gene dependency on specific cancer therapeutic drug targets as applied to cell samples derived from and analyzed in regard to individual subjects with cancer. This platform creates the opportunity for precision cancer treatment using comprehensive functional genomic profiling in an unbiased and patient-specific manner by uncovering drug target dependencies not identified by conventional genomic profiling.

In one aspect, provided herein are methods of treating cancer in a subject in need thereof, the methods comprising administering to the subject a therapeutic molecule selected from a compilation of therapeutic molecules using personalized profiling, e.g., a CRISPR-based screen. In some instances, the therapeutic molecules are selected by a screening method comprising: establishing cell cultures from cancer patients; modifying cancer cells from the subject by altering a function, e.g., knocking down, of a plurality of genes using a library of gene modulatory reagents, where each gene in the plurality of genes encodes a protein target of a therapeutic molecule in the compilation of therapeutic molecules; accessing cell function, viability, or proliferation; identifying the protein targets, e.g., by sequencing; and selecting the therapeutic molecule acting on the protein targets. In some instances, the protein target is identified if altering the function of the target gene impairs cancer cell viability or proliferation rate during in vitro propagation.

In another aspect, the present disclosure provides methods of perturbing gene function in a plurality of modified cancer cells from a subject having cancer, the methods comprising delivering a library of gene modulatory reagents to a sample of cancer cells from the subject to generate the plurality of modified cancer cells. In this instance, each modified cancer cell harbors one or more of the gene modulatory reagents, and each gene modulatory reagent is capable of perturbing the function of a gene that encodes a protein target from a library of protein targets.

In one aspect, the present disclosure provides a library comprising a plurality of gene perturbation reagents, where each gene perturbation reagent is capable of knocking down the function of a gene that encodes a protein target from a library of protein targets.

In another aspect, the present disclosure provides a compilation comprising a plurality of modified cancer cells, where each modified cancer cell harbors one or more gene modulatory reagents, and each gene modulatory reagent is capable of knocking down the function of a gene that encodes a protein target from a library of protein targets.

In one aspect, the present disclosure provides a method of evaluating the functional effect of perturbing gene activity in cancer cells from a subject, the method comprising sequencing a plurality of modified cancer cells, where each modified cancer cell harbors one or more gene modulatory reagents, and each gene perturbation reagent capable of knocking down the function of a gene that encodes a protein target in a library of protein targets. In this instance, a gene perturbation reagent that impairs cell viability or proliferation rate has fewer sequence reads than a gene perturbation reagent that does not significantly impair cell viability or proliferation rate.

In another aspect, the present disclosure provides a method of treating cancer in a subject in need thereof, the method comprising administering to the subject a therapeutic molecule selected from a compilation of therapeutic molecules, where the cancer of the subject has been determined to be susceptible to the selected therapeutic molecule by a screening method. In this instance, the screening method comprises: contacting a sample of cancer cells from the subject with a library of gene modulatory reagents to generate a plurality of modified cancer cells, where each modified cancer cell harbors one or more of the gene modulatory reagents, and where the one or more of the gene modulatory reagents are capable of knocking down the function of a gene that encodes a protein target of a therapeutic molecule in the library of therapeutic molecules; and sequencing the plurality of modified cancer cells, where a gene modulatory reagent that impairs cell viability or proliferation rate has fewer sequence reads than a gene modulatory reagent that does not impair cell viability or proliferation rate, and where the one or more of the gene modulatory reagents capable of knocking down the function of the gene comprise sequence complementarity to a gene that encodes for the protein targeted by the selected therapeutic molecule. In some embodiments, contacting the sample of cancer cells from the subject with the library of gene modulatory reagents comprises adding the library of gene modulatory reagents to a culture medium in which the cancer cells are being maintained ex vivo. In some embodiments, the gene modulatory reagents comprise infectious particles used to deliver nucleic acid vector sequence encoding the gene modulatory reagents.

In any of the methods described herein, in some embodiments, the one or more gene modulatory reagents knock down the function of a plurality of genes. In some cases, the methods do not comprise cleavage of genomic DNA of the cancer cells modified by the one or more gene modulatory reagents. Example gene modulatory reagents include CRISPR interference (CRISPRi) reagents, for instance, as further described herein. In example embodiments, the methods do not employ use of a catalytically active CAS endonuclease.

### Cancers

Cancer is a disease where abnormal cells in the body divide uncontrollably and can invade other tissues. In some instances, cancer is caused by changes in gene(s) which in turn affect the function of proteins encoded by those genes, e.g., genes that control cell growth, division, and/or survival. Cancer can occur in any part of the body. Examples of cancer include, but are not limited to, lung cancer, prostate cancer, colorectal cancer, and hematologic malignancies.

Hematological malignancy, which is also known as blood cancer or heme cancer, is a type of cancer that begins in blood-forming tissue, e.g., the bone marrow or cells of the immune system. Three main types of hematological malignancies are myeloid neoplasms, lymphoid neoplasm, and histiocytic and dendritic cell neoplasms. Myeloid neoplasms are conditions where myeloid cells, which are cells that give rise to red blood cells, granulocytes, monocytes, and platelets, become cancerous. Lymphoid neoplasms are conditions where lymphoid cells, which are cells that give rise to lymphocytes, e.g., T cells and B cells, and natural killer cells, become cancerous. Histiocytic and dendritic cell neoplasms are rare and characterized by shared morphologic, immunophenotypic, and ultrastructural characteristics of mature histiocytic/dendritic neoplasms. In some instances, histiocytic and dendritic cell neoplasms arise de novo or in association with lymphoid neoplasms or myeloid neoplasms. In some instances, three types of hematological malignancies can be categorized based on the affected cells/organs/tissues, and these three types of hematological malignancies are leukemia, lymphoma, and myeloma. Leukemia typically originates in the blood or bone marrow, and this is a condition in which the bone marrow produces too many white blood cells, but not enough red blood cells and platelets. Lymphoma usually starts in lymph nodes or other parts of the lymphatic system. Two main subtypes of lymphoma are Hodgkin's lymphoma and Non-Hodgkin's lymphoma. Myeloma usually originates in the plasma cells, which is a type of white blood cells that produce large amounts of specific antibodies.

In some embodiments, the present disclosure provides a method of treating malignancies in a subject in need thereof, the method comprising administering to the subject a therapeutic molecule selected from a compilation of therapeutic molecules, where the cancer of the subject has been determined to be susceptible to the selected therapeutic molecule by a screening method. In some embodiments, the malignancies comprise carcinomas. In some embodiments, the carcinoma is a squamous cell carcinoma, an adenocarcinoma, a renal cell carcinoma, a ductal carcinoma in situ, an invasive ductal carcinoma, a transitional cell carcinoma, or a basal cell carcinoma. In some embodiments, the malignancies comprise sarcomas. In some embodiments, the sarcoma is a soft tissue carcinoma. In some embodiments, the sarcoma is a bone sarcoma. In some embodiments, the soft tissue carcinoma is a gastrointestinal stromal tumor, a liposarcoma, a leiomyosarcoma, an undifferentiated pleomorphic sarcoma, a synovial sarcoma, a malignant peripheral nerve sheath tumor, a desmoid tumor, a solitary fibrous tumor, a hemoangiopericytoma, a fibrosarcoma, a vascular sarcoma, an epithelioid sarcoma, an alveolar soft part sarcoma, a clear cell sarcoma, a melanoma, or an extraskeletal myxoid chondrosarcoma.

In some embodiments, the present disclosure provides a method of treating hematological malignancies in a subject in need thereof, the method comprising administering to the subject a therapeutic molecule selected from a compilation of therapeutic molecules, where the cancer of the subject has been determined to be susceptible to the selected therapeutic molecule by a screening method. In some embodiments, the hematological malignancies comprise myeloid neoplasms, lymphoid neoplasm, or histiocytic and dendritic cell neoplasms. In some embodiments, the cancer of the subject comprises a hematologic malignancy. In some embodiments, the hematologic malignancy comprises a leukemia, a lymphoma, a myeloma, or a combination thereof. In some embodiments, the leukemia comprises Acute Myeloid Leukemia (AML).

In some embodiments, the myeloid neoplasms comprise a condition listed in TABLE 5. In some embodiments, the myeloid neoplasms comprise myeloproliferative neoplasms, mastocytosis, myeloid/lymphoid neoplasms with eosinophilia and gene rearrangement, myelodysplastic/myeloproliferative neoplasms, myelodysplastic syndromes, myeloid neoplasms with germline predisposition, acute myeloid leukemia and related neoplasms, blastic plasmacytoid dendritic cell neoplasm, or acute leukemias of ambiguous lineage.

In some embodiments, the myeloproliferative neoplasms comprise chronic myeloid leukemia, BCR-ABL1-positive, chronic neutrophilic leukemia, polycythemia vera, primary myelofibrosis, essential thrombocythemia, chronic eosinophilic leukemia, not otherwise specified, or myeloproliferative neoplasm, unclassifiable.

In some embodiments, the mastocytosis comprises cutaneous mastocytosis, systemic mastocytosis, or mast cell sarcoma. In some embodiments, the systemic mastocytosis comprises indolent systemic mastocytosis, smoldering systemic mastocytosis, systemic mastocytosis with an associated hematological neoplasm, aggressive systemic mastocytosis, or mast cell leukemia.

In some embodiments, the myeloid/lymphoid neoplasms with eosinophilia and gene rearrangement comprises myeloid/lymphoid neoplasms with PDGFRA rearrangement, myeloid/lymphoid neoplasms with PDGFRB rearrangement, myeloid/lymphoid neoplasms with FGFR1 rearrangement, or myeloid/lymphoid neoplasms with PCM1-JAK2.

In some embodiments, the myelodysplastic/myeloproliferative neoplasms comprise chronic myelomonocytic leukemia, atypical chronic myeloid leukemia, BCR-ABL1-negative, juvenile myelomonocytic leukemia, myelodysplastic/myeloproliferative neoplasm with ring sideroblasts and thrombocytosis, or myelodysplastic/myeloproliferative neoplasm, unclassifiable.

In some embodiments, the myelodysplastic syndromes comprise myelodysplastic syndrome with single lineage dysplasia, myelodysplastic syndrome with multilineage dysplasia, myelodysplastic syndrome with ring sideroblasts, myelodysplastic syndrome with excess blasts, myelodysplastic syndrome with isolated del (5q), myelodysplastic syndrome, unclassifiable, or refractory cytopenia of childhood (provisional entity). In some embodiments, the myelodysplastic syndrome with ring sideroblasts comprises myelodysplastic syndrome with ring sideroblasts and single lineage dysplasia, or myelodysplastic syndrome with ring sideroblasts and multilineage dysplasia.

In some embodiments, the myeloid neoplasms with germline predisposition comprises myeloid neoplasms with germline predisposition without a preexisting disorder or organ dysfunction, myeloid neoplasms with germline predisposition and preexisting platelet disorders, or myeloid neoplasms with germline predisposition and other organ dysfunction. In some embodiments, the myeloid neoplasms with germline predisposition without a preexisting disorder or organ dysfunction comprises acute myeloid leukemia with germline CEBPA mutation, or myeloid neoplasms with germline DDX41 mutation. In some embodiments, the myeloid neoplasms with germline predisposition and preexisting platelet disorders comprises myeloid neoplasms with germline RUNX1 mutation, myeloid neoplasms with germline ANKRD26 mutation, or myeloid neoplasms with germline ETV6 mutation. In some embodiments, the myeloid neoplasms with germline predisposition and other organ dysfunction comprises myeloid neoplasms with germline GATA2 mutation, myeloid neoplasms associated with bone marrow failure syndromes, myeloid neoplasms associated with telomere biology disorders, juvenile myelomonocytic leukemia associated with neurofibromatosis, Noonan syndrome or Noonan syndrome-like disorders, or myeloid neoplasms associated with Down syndrome.

In some embodiments, the acute myeloid leukemia and related neoplasms comprises acute myeloid leukemia with recurrent genetic abnormalities, acute myeloid leukemia with myelodysplasia-related changes, therapy-related myeloid neoplasms, acute myeloid leukemia, not otherwise specified, myeloid sarcoma, or myeloid proliferations related to Down syndrome. In some embodiments, the acute myeloid leukemia with recurrent genetic abnormalities comprises acute myeloid leukemia with t (8;21) (q22;q22.1); RUNX1-RUNXIT1, acute myeloid leukemia with inv (16) (p13.1q22) ort (16;16) (p13.1;q22); CBFB-MYH11, acute promyelocytic leukemia with PML-RARA, acute myeloid leukemia with t (9;11) (p21.3;q23.3); MLLT3-KMT2A, acute myeloid leukemia with t (6;9) (p23;q34.1); DEK-NUP214, acute myeloid leukemia with inv (3) (q21.3q26.2) or t (3;3) (q21.3;q26.2); GATA2, MECOM, acute myeloid leukemia (megakaryoblastic) with t (1;22) (p13.3;q13.3); RBM15-MKL1, acute myeloid leukemia with BCR-ABL1 (provisional entity), acute myeloid leukemia with mutated NPM1, acute myeloid leukemia with biallelic mutations of CEBPA, or acute myeloid leukemia with mutated RUNX1 (provisional entity).

In some embodiments, the acute myeloid leukemia, not otherwise specified comprises acute myeloid leukemia with minimal differentiation, acute myeloid leukemia without maturation, acute myeloid leukemia with maturation, acute myelomonocytic leukemia, acute monoblastic/monocytic leukemia, pure erythroid leukemia, acute megakaryoblastic leukemia, acute basophilic leukemia, or acute panmyelosis with myelofibrosis.

In some embodiments, the myeloid proliferations related to Down syndrome comprises transient abnormal myelopoiesis or myeloid leukemia associated with Down syndrome.

In some embodiments, the acute leukemias of ambiguous lineage comprises acute undifferentiated leukemia, mixed phenotype acute leukemia with t (9;22) (q34.1;q11.2); BCR-ABL1, mixed phenotype acute leukemia with t (v;11q23.3); KMT2A rearranged, mixed phenotype acute leukemia, B/myeloid, not otherwise specified, or mixed phenotype acute leukemia, T/myeloid, not otherwise specified.

In some embodiments, the lymphoid neoplasms comprise a condition listed in TABLE 6. In some embodiments, the lymphoid neoplasms comprise precursor lymphoid neoplasms, mature B-cell neoplasms, mature T- and NK-cell neoplasms, Hodgkin lymphoma, or posttransplant lymphoproliferative disorders (PTLD).

In some embodiments, the precursor lymphoid neoplasms comprise B-lymphoblastic leukemia/lymphoma, T-lymphoblastic leukemia/lymphoma, or natural killer (NK) cell lymphoblastic leukemia/lymphoma (provisional entity).

In some embodiments, the B-lymphoblastic leukemia/lymphoma comprises B-lymphoblastic leukemia/lymphoma, not otherwise specified or B-lymphoblastic leukemia/lymphoma with recurrent genetic abnormalities. In some embodiments, the B-lymphoblastic leukemia/lymphoma with recurrent genetic abnormalities comprises B-lymphoblastic leukemia/lymphoma with t (9;22) (q34.1;q11.2); BCR-ABL1, B-lymphoblastic leukemia/lymphoma with t (v;11q23.3); KMT2A rearranged, B-lymphoblastic leukemia/lymphoma with t (12;21) (p13.2;q22.1); ETV6-RUNX1, B-lymphoblastic leukemia/lymphoma with hyperdiploidy, B-lymphoblastic leukemia/lymphoma with hypodiploidy, B-lymphoblastic leukemia/lymphoma with t (5;14) (q31.1;q32.3); IL3-IGH, B-lymphoblastic leukemia/lymphoma with t (1;19) (q23;p13.3); TCF3-PBX1, B-lymphoblastic leukemia/lymphoma, BCR-ABL1-like (provisional entity), or B-lymphoblastic leukemia/lymphoma with iAMP21 (provisional entity).

In some embodiments, the T-lymphoblastic leukemia/lymphoma comprises early T-cell precursor lymphoblastic leukemia (provisional entity).

In some embodiments, the mature B-cell neoplasms comprises chronic lymphocytic leukemia/small lymphocytic lymphoma, monoclonal B-cell lymphocytosis, B-cell prolymphocytic leukemia, splenic marginal zone lymphoma, hairy cell leukemia, splenic B-cell lymphoma/leukemia, unclassifiable, lymphoplasmacytic lymphoma, IgM monoclonal gammopathy of undetermined significance, heavy-chain diseases, plasma cell neoplasms, extranodal marginal zone lymphoma of mucosa-associated lymphoid tissue (MALT lymphoma), nodal marginal zone lymphoma, follicular lymphoma, pediatric-type follicular lymphoma, large B-cell lymphoma with IRF4 rearrangement (provisional entity), primary cutaneous follicle center lymphoma, mantle cell lymphoma, diffuse large B-cell lymphoma, not otherwise specified, T-cell/histiocyte-rich large B-cell lymphoma, primary diffuse large B-cell lymphoma of the central nervous system, primary cutaneous diffuse large B-cell lymphoma, leg type, EBV-positive diffuse large B-cell lymphoma, not otherwise specified, EBV-positive mucocutaneous ulcer (provisional entity), diffuse large B-cell lymphoma associated with chronic inflammation, lymphomatoid granulomatosis, primary mediastinal (thymic) large B-cell lymphoma, intravascular large B-cell lymphoma, ALK-positive large B-cell lymphoma, plasmablastic lymphoma, primary effusion lymphoma, HHV8-associated lymphoproliferative disorders, Burkitt lymphoma, Burkitt-like lymphoma with 11q aberration (provisional entity), high-grade B-cell lymphoma, or B-cell lymphoma, unclassifiable, with features intermediate between diffuse large B-cell lymphoma and classical Hodgkin lymphoma.

In some embodiments, the splenic B-cell lymphoma/leukemia, unclassifiable comprises splenic diffuse red pulp small B-cell lymphoma (provisional entity) or hairy cell leukemia-variant (provisional entity). In some embodiments, the lymphoplasmacytic lymphoma comprises Waldenström macroglobulinemia.

In some embodiments, the heavy-chain diseases comprise u heavy-chain disease, γ heavy-chain disease, or a heavy-chain disease.

In some embodiments, the plasma cell neoplasms comprise non-IgM monoclonal gammopathy of undetermined significance (IgG/A), plasma cell myeloma (a.k.a. multiple myeloma), plasma cell myeloma variants, plasmacytoma, monoclonal immunoglobulin deposition diseases, or plasma cell neoplasms with associated paraneoplastic syndrome. In some embodiments, the plasma cell myeloma variants comprise smoldering (asymptomatic) plasma cell myeloma, non-secretory myeloma, or plasma cell leukemia. In some embodiments, the plasmacytoma comprises solitary plasmacytoma of bone or extraosseous plasmacytoma. In some embodiments, the monoclonal immunoglobulin deposition diseases comprise primary amyloidosis or light chain and heavy chain deposition diseases. In some embodiments, the plasma cell neoplasms with associated paraneoplastic syndrome comprises POEMS syndrome or TEMPI syndrome.

In some embodiments, the nodal marginal zone lymphoma comprises pediatric nodal marginal zone lymphoma (provisional entity).

In some embodiments, the follicular lymphoma comprises testicular follicular lymphoma, in situ follicular neoplasia, or duodenal-type follicular lymphoma.

In some embodiments, the mantle cell lymphoma comprises leukemic non-nodal mantle cell lymphoma or in situ mantle cell neoplasia.

In some embodiments, the diffuse large B-cell lymphoma, not otherwise specified comprises germinal center B-cell type or activated B-cell type.

In some embodiments, the diffuse large B-cell lymphoma associated with chronic inflammation comprises fibrin-associated diffuse large B-cell lymphoma.

In some embodiments, the HHV8-associated lymphoproliferative disorders comprises multicentric Castleman disease, HHV8-positive diffuse large B-cell lymphoma, not otherwise specified (provisional entity), or HHV8-positive germinotropic lymphoproliferative disorder.

In some embodiments, the high-grade B-cell lymphoma comprises high-grade B-cell lymphoma with MYC and BCL2 and/or BCL6 rearrangements or high-grade B-cell lymphoma, not otherwise specified.

In some embodiments, the mature T- and NK-cell neoplasms comprises T-cell prolymphocytic leukemia, T-cell large granular lymphocytic leukemia, chronic lymphoproliferative disorder of NK cells (provisional entity), aggressive NK-cell leukemia, EBV-positive T-cell and NK-cell lymphoproliferative diseases of childhood, adult T-cell leukemia/lymphoma, extranodal NK-/T-cell lymphoma, nasal type, intestinal T-cell lymphoma, hepatosplenic T-cell lymphoma, subcutaneous panniculitis-like T-cell lymphoma, mycosis fungoides, Sézary syndrome, primary cutaneous CD30+ T-cell lymphoproliferative disorders, primary cutaneous peripheral T-cell lymphomas, rare subtypes, peripheral T-cell lymphoma, not otherwise specified, angioimmunoblastic T-cell lymphoma and other nodal lymphomas of T follicular helper cell origin, anaplastic large-cell lymphoma, or breast implant-associated anaplastic large-cell lymphoma (provisional entity).

In some embodiments, the EBV-positive T-cell and NK-cell lymphoproliferative diseases of childhood comprises systemic EBV-positive T-cell lymphoma of childhood, chronic active EBV infection of T- and NK-cell type, systemic form, hydroa vacciniforme-like lymphoproliferative disorder, or severe mosquito bite allergy.

In some embodiments, the intestinal T-cell lymphoma comprises enteropathy-associated T-cell lymphoma, monomorphic epitheliotropic intestinal T-cell lymphoma, intestinal T-cell lymphoma, not otherwise specified, or indolent T-cell lymphoproliferative disorder of the gastrointestinal tract (provisional entity).

In some embodiments, the primary cutaneous CD30+ T-cell lymphoproliferative disorders comprises lymphomatoid papulosis or primary cutaneous anaplastic large cell lymphoma.

In some embodiments, the primary cutaneous peripheral T-cell lymphomas, rare subtypes comprise primary cutaneous γδ T-cell lymphoma, primary cutaneous CD8+ aggressive epidermotropic cytotoxic T-cell lymphoma (provisional entity), primary cutaneous acral CD8+ T-cell lymphoma (provisional entity), or primary cutaneous CD4+ small/medium T-cell lymphoproliferative disorder (provisional entity).

In some embodiments, the angioimmunoblastic T-cell lymphoma and other nodal lymphomas of T follicular helper cell origin comprises angioimmunoblastic T-cell lymphoma, follicular T-cell lymphoma (provisional entity), or nodal peripheral T-cell lymphoma with T follicular helper phenotype (provisional entity).

In some embodiments, the anaplastic large-cell lymphoma comprises anaplastic large-cell lymphoma, ALK-positive or anaplastic large-cell lymphoma, ALK-negative.

In some embodiments, the Hodgkin lymphoma comprises nodular lymphocyte predominant Hodgkin lymphoma or classical Hodgkin lymphoma. In some embodiments, the classical Hodgkin lymphoma comprises nodular sclerosis classical Hodgkin lymphoma, lymphocyte-rich classical Hodgkin lymphoma, mixed cellularity classical Hodgkin lymphoma, or lymphocyte-depleted classical Hodgkin lymphoma.

In some embodiments, the posttransplant lymphoproliferative disorders (PTLD) comprises plasmacytic hyperplasia PTLD, infectious mononucleosis PTLD, florid follicular hyperplasia PTLD, polymorphic PTLD, monomorphic PTLD, or classical Hodgkin lymphoma PTLD.

In some embodiments, the monomorphic PTLD comprises monomorphic B-cell PTLD or monomorphic T/NK-cell PTLD.

In some embodiments, the histiocytic and dendritic cell neoplasms comprise a condition listed in TABLE 7. In some embodiments, the histiocytic and dendritic cell neoplasms comprise histiocytic sarcoma, tumors derived from Langerhans cells, indeterminate dendritic cell tumor, interdigitating dendritic cell sarcoma, follicular dendritic cell sarcoma, fibroblastic reticular cell tumor, disseminated juvenile xanthogranuloma, or Erdheim-Chester disease. In some embodiments, the tumors derived from Langerhans cells comprise Langerhans cell histiocytosis or Langerhans cell sarcoma. In some embodiments, the follicular dendritic cell sarcoma comprises inflammatory pseudotumor-like follicular/fibroblastic dendritic cell sarcoma.

Genomic instability is a frequent characteristic in many types of tumor cells. The molecular nature of this genomic instability in tumor cells is complex and involves significant influences from DNA damage, tumor-specific DNA repair defects, and failure to halt or stall the cell cycle prior to damaged DNA being passed on to daughter cells during cellular proliferation. Normal metabolic activities and environmental factors, including radiation, can cause DNA damage. If not repaired, this DNA damage cell alter or impair an ability of a cell transcribe genetic loci near or within a site of DNA damage. Most healthy cells possess an ability to sense and repair damaged DNA. Cellular machinery is capable of facilitating DNA damage repair such as breaks in chromosomal DNA. DNA damage repair processes often either effectively repair lesions in DNA or instruct a cell to undergo apoptosis should irreparable DNA damage occur including double-stranded DNA breaks or DNA crosslinkages. Through these active DNA damage repair processes prevalent in healthy cells, damaged cells can be prevented from proceeding to conversion into malignant cells, or pre-malignant cells can be repressed from a pathway toward malignancy and uncontrolled cellular proliferation. A defective DNA damage repair response in a cell can promote the clonal evolution of cells towards a cancer state by the accumulation of genetic driver aberrations, including alterations in gene copy number, genetic rearrangements, and oncogenic mutations. The tumor suppressor gene p53 controls a variety of DNA-damage-response (DDR) cellular mechanisms. p53 is known to play a prominent role as a facilitator of DNA repair by stopping the cells cycle for a long enough period to allow cellular repair machineries to restore genome stability. This is accomplished through holding the cell cycle at the Gl/S regulation point. p53 can also activate DNA repair proteins when DNA damage has been detected. If the DNA damage is irreparable, p53 can initiate cellular apoptosis, eliminating a cell which may otherwise remain as a pre-cancerous or cancerous cell.

In some embodiments of methods of treating cancer described herein, tumor cells do not maintain an active DDR as part of their functional cellular machinery. In some embodiments of methods of treating cancer described herein, tumor cells maintain an active DDR as part of their functional cellular machinery. In some embodiments, tumor cells originating from myeloid neoplasms maintain an active DDR as part of their functional cellular machinery. In some embodiments, tumor cells originating from lymphoid neoplasms maintain an active DDR as part of their functional cellular machinery. In some embodiments, tumor cells originating from histiocytic and dendritic cell neoplasms maintain an active DDR as part of their functional cellular machinery. In some embodiments, the tumor cells that maintain an active DDR maintain an active p53-dependent DDR. In some embodiments described herein, methods of genetic modification that create targeted DNA cleavage (e.g. CRISPRn) would be toxic to the tumor cells maintaining an active p53-dependent DDR. In some embodiments described herein, methods of genetic modification that create targeted DNA cleavage (e.g. CRISPRn) would be less effective at knocking down gene function in tumor cells maintaining an active p53-dependent DDR than methods that knock down gene function without creating targeted DNA cleavage. In some embodiments described herein, methods of modifying cancer cells from the subject by knocking down the function of a plurality of genes without targeted DNA cleavage of one or more genomic loci in cancer cells maintaining and active p53-dependent DDR can allow for analysis of functional consequences of gene repression in these cancer cells. In some embodiments, the use of CRISPRi to modify cancer cells by knocking down gene function without targeted DNA cleavage allows for the functional analysis of growth, proliferation, and cellular maintenance in cancer cells with an active p53-dependent DDR. In some embodiments, the use of RNAi to modify cancer cells by knocking down gene function without targeted DNA cleavage allows for the functional analysis of growth, proliferation, and cellular maintenance in cancer cells with an active p53-dependent DDR.

### Genetic Screening Using Cancer Cells

Provided herein are methods and compositions to identify therapeutic molecules for use in a treatment of a subject in need thereof using personalizing profiling methods that are based on genetic screening established from cancer cells. In some embodiments, the personalizing profiling methods comprise establishing cell cultures obtained from clinical samples of cancer patients; transducing cancer cell cultures with gene modification systems to alter function of a plurality of genes, where each gene in the plurality of genes encodes a protein target of a therapeutic molecule in the library of therapeutic molecules; maintaining the cell cultures for a period of time; collecting cells to assess cell viability or proliferation; identifying target hits; and selecting the therapeutic molecule or molecules corresponding to the target hits for treatment in each cancer patient.

In some embodiments, the cancer cells are obtained from the subject. In some embodiments, the cancer cells from the subject comprise primary cancer cells. In some embodiments, the cancer cells from the subject comprise cells from an established cancer cell line. In some embodiments, the cancer cells from the subject comprise cells from an immortalized cell line.

In some embodiments, genetic screening comprises gene modification systems. In some embodiments, the gene modification systems comprise gene modulatory reagents. In some embodiments, the gene modulatory reagents are configured for knocking down the function of the plurality of genes. In some embodiments, the gene modulatory reagents are configured for knocking down the function of a selected gene. In some embodiments, the gene modulatory reagents comprise CRISPR-based gene modification, Transcription activator-like effector (TALE) repression, or post-transcriptional knocking down of gene mRNA function. In some embodiments, the CRISPR-based gene modification comprises CRISPR interference (CRISPRi). In some embodiments, the post-transcriptional knocking down of gene mRNA function comprises using RNA interference (RNAi). In some embodiments, the gene modulatory reagents used for knocking down the function of the plurality of genes are delivered by a viral vector comprising gene modification systems. In some embodiments, the gene modification systems comprise CRISPR-based gene modification. In some embodiments, the CRISPR-based gene modification comprises CRISPRi.

### Establishing Cancer Cell Culture

Cells from a subject may be isolated and maintained ex vivo. As described herein, cells isolated from a subject and maintained ex vivo may be used in methods of treating cancer, in methods of perturbing gene function, in methods of assessing efficacy of a prior or ongoing cancer treatment, in methods of evaluating the functional effect of perturbing gene activity in cancer cells from a subject, or as part of a compilation comprising a plurality of modified cancer cells. In some embodiments, cells isolated from a subject and maintained ex vivo comprise oncogenic cells. In some embodiments, cells isolated from a subject and maintained ex vivo comprise cancer cells. In some embodiments, cells isolated from a subject and maintained ex vivo comprise primary cancer cells. In some embodiments, cells isolated from a subject and maintained ex vivo comprise a heterogeneous population of primary cancer cells. In some embodiments, the portions of the heterogeneous population of primary cancer cell may comprises distinct oncogenic or tumor suppressive genomic mutations. In some embodiments, cells isolated from a subject and maintained ex vivo comprise a heterogenous mixture of healthy cells and primary cancer cells. In some embodiments, cells isolated from a subject and maintained ex vivo are obtained from a biopsy of the subject. In some embodiments, the biopsy is taken from a site of a primary tumor. In some embodiments, the biopsy is taken from a secondary tumor site. In some embodiments, the biopsy is taken from a site of tumor metastasis. In some embodiments, cells isolated from a subject and maintained ex vivo are obtained from a collection of a blood sample from the subject. In some embodiments, cells isolated from a subject and maintained ex vivo are obtained from a preparation of a plasma sample from the subject. In some embodiments, cells isolated from a subject and maintained ex vivo are obtained from a buffy coat layer in a preparation of a plasma sample from the subject.

Cells from a subject that have been isolated may be maintained ex vivo using a variety of cell maintenance techniques. In some embodiments, cells isolated from the subject are maintained by establishing a cell culture. In some embodiments, fresh samples of peripheral blood or bone marrow from subjects are obtained and maintained by establishing a cell culture. In some embodiments, viably frozen samples of peripheral blood or bone marrow from subjects are obtained and stored prior to maintaining the cells by thawing and establishing a cell culture. In some embodiments, the peripheral blood or bone marrow cells are taken from the subject prior to a diagnosis. In some embodiments, the peripheral blood or bone marrow cells are taken from the subject prior to an AML diagnosis. In some embodiments, the peripheral blood or bone marrow cells are taken from the subject prior to initiation of a treatment. In some embodiments, the peripheral blood or bone marrow cells are taken from the subject during an active stage of treatment. In some embodiments, the peripheral blood or bone marrow cells are taken from the subject following completion of a treatment. In some embodiments, the peripheral blood or bone marrow cells are taken from the subject at multiple time points. In some embodiments, the peripheral blood or bone marrow cells are stored viably frozen until thawed and maintained by establishing a cell culture. In some embodiments, the peripheral blood or bone marrow cells taken from the subject at multiple time points comprise one or more samples taken prior to initiation of a treatment and one or more samples taken following completion of a treatment. In some embodiments, results of gene dependency determination for cells from a subject are determined using CRISPRi gene modulation prior to initiation of a treatment and following completion of a treatment and the results are compared. In some embodiments, the cell culture comprises cancer cells derived from the subject. In some embodiments, the cancer cells are metastatic cancer cells. In some embodiments, the cell culture maintains cells in a growth medium compatible with maintenance, cell division, and growth of the isolated cells. In some embodiments, the cell culture maintains cells for growth on a sterile cell culture plate under normoxic growth conditions. In some embodiments, the cell culture maintains cells for growth on a sterile cell culture plate under hypoxic growth conditions. In some embodiments, the cell culture maintains cells according to non-adherent cell culture protocols known in the art for growing myeloid neoplasms, lymphoid neoplasm, or histiocytic and dendritic cell neoplasms. In some embodiments, the cell culture maintains cells for growth with supplemental growth factors included in the cell culture medium. In some embodiments, cells are maintained ex vivo in preparation for methods involving genetic modification of the cells. In some embodiments, cells are maintained ex vivo in preparation for methods involving targeted knockdown of gene function in the cells. In some embodiments, cells are maintained ex vivo during methods involving genetic modification and targeted knockdown of gene function in the cells, such as the use of CRISPRi. In some embodiments, cells are maintained ex vivo after methods involving genetic modification of the cells. In some embodiments, cells are maintained ex vivo after methods involving targeted knockdown of gene function in the cells. In some embodiments, the extent of cell growth or cell proliferation after methods involving genetic modification of the cells or targeted knockdown of gene function in the cells is quantitated following a period of maintenance of the cells ex vivo.

In one aspect, provided herein are methods of propagating the plurality of genetically modified cells. For example, the genetically modified cells are modified using a CRISPRi gene editing system or RNAi as described herein. The cells may be modified from primary cancer cells. The cells may be modified from a cancer cell line. In some embodiments, the plurality of modified cells is propagated in 2D format in vitro, 3D format in vitro, or in vivo. Non-limiting examples of the 3D in vitro format include, but are not limited to, propagating cells embedded in sponge matrices (e.g., collagen-based), scaffolds, extracellular matrix (ECM) conditions such as basement membrane extract or Matrigel, in suspension, in organoid culture, or in microfluidic platforms. Exemplary materials constituting 3D in vitro format for cell propagation include, but are not limited to, collagen, gelatin, elastin, fibronectin, laminin, vitronectin, poly-lysine, poly-L-ornithine, silicone, polysaccharide polymers such as alginate, agar, dextran, carrageenan, chitosan, pectin, cellulose, gellan gum, xanthan gum, pullulan, glycosaminoglycan and any fragmented or derivative forms, hyaluronic acid, heparan, heparin, dermatan, chondroitin, or any hydrogel or biocompatible polymer. For in vitro approaches with cancer cells, the cancer cells are maintained under conditions that support bulk cell survival and allow selective pressure from induced mutations. For in vivo approaches, a propagation technique is selected which maximizes engraftment efficiency and survival. In some embodiments, in vivo cell propagation includes, but is not limited to, patient derived xenograft via either heterotopic implantation or orthotopic implantation. Additionally, for in vivo approaches, modified cancer cells may be implanted orthotopically (e.g., within the pancreas, for a pancreatic-origin tumor) or ectopically (e.g., subcutaneously, for a pancreatic origin tumor). In some embodiments, no cell sorting is used on peripheral blood or bone marrow cells obtained from the subject prior to or during cell maintenance through establishing a cell culture. In some embodiments, there are no steps of removing non-cancerous cells from peripheral blood or bone marrow cell samples from the subject prior to or during cell maintenance through establishing a cell culture. In some embodiments, there are no steps of enriching cancerous cells for blast subset from peripheral blood or bone marrow cell samples from the subject prior to or during cell maintenance through establishing a cell culture. In some embodiments, cells are sorted prior to or during cell maintenance stages. In some embodiments, cells are separated prior to or during cell maintenance stages. In some embodiments, some non-cancerous cells are removed from the cell sample prior to or during cell maintenance stages. In some embodiments, cell separation is used to separate blast cells from normal cells. In some embodiments, cell samples from the subject undergo an enrichment for blast subset prior to or during cell maintenance stages. In some embodiments, one or more cells from the cell sample derived from the subject are assayed for blast cells. In some embodiments, assaying for blast cells entails assaying one or more cells from the cell sample for particular markers of cancerous cells. In some embodiments, assaying for blast cells in one or more cells from the cell sample will indicate a proportion of cancer calls present in the entire cell sample. In some embodiments, CD34 and/or CD33 expression are assayed in one or more cells from the cell sample as representative cancer markers used for the blast subset. In some embodiments, assaying for blast cells in the cell sample is completed prior to CRISPR-based gene modulation being performed on cells from the subject. In some embodiments, assaying for blast cells in the cell sample is completed prior to viably freezing cells from the cell sample for later analysis using CRISPR-based gene modulation.

### Methods of Genetic Modification

An exemplary method for generating a plurality of modified cancer cells from a subject comprises: delivering a library of gene modulatory reagents to a sample of cancer cells from the subject to generate the plurality of modified cancer cells, wherein each modified cancer cell harbors one or more of the gene modulatory reagents, and each gene modulatory reagent is capable of knocking down or knocking out the function of a gene that encodes a protein target from a compilation of protein targets.

In some embodiments, the method for generating the plurality of modified cancer cells comprises a CRISPR/endonuclease-based gene editing system, CRISPRi, Transcription activator-like effector (TALE) repression, Zinc-finger protein based repression, or RNAi. For instance, one or more of the gene modulatory reagents comprises a gRNA sequence comprising homology to at least a portion of the gene whose function is knocked out in the modified cancer cell. The gRNA may comprise homology to about 10 to about 50 contiguous nucleotides of the gene. The homology may be at least about 90% sequence identity. In some embodiments, one or more of the gene modulatory reagents each comprise a gRNA sequence comprising at least about 90% identity to a sequence selected from SEQ ID NOS: 55-1669. In some embodiments, one or more of the gene modulatory reagents each comprise a gRNA sequence comprising at least about 90% identity to a sequence selected from SEQ ID NOS: 55-459. In some embodiments, one or more of the gene modulatory reagents each comprise a gRNA sequence comprising at least about 90% identity to a sequence selected from SEQ ID NOS: 460-1669. In some embodiments, one or more of the gene modulatory reagents each comprise a gRNA sequence comprising at least about 90% identity to a sequence selected from SEQ ID NOS: 1670-8119. In some embodiments, one or more of the gene modulatory reagents comprising a gRNA sequence comprising at least about 90% identity to a sequence selected from SEQ ID NOS: 1670-8119 target one or more kinase genes. In some embodiments, one or more of the gene modulatory reagents each comprise a gRNA sequence comprising at least about 90% identity to a sequence selected from SEQ ID NOS: 8120-12949. In some embodiments, one or more of the gene modulatory reagents comprising a gRNA sequence comprising at least about 90% identity to a sequence selected from SEQ ID NOS: 8120-12949 target one or more IgSF genes. A sequence encoding the gRNA may be positioned within a vector, e.g., for viral delivery as discussed herein. In some embodiments, the gRNA is expressed following transcription of genetic material from the vector.

The method for generating modified cancer cells may comprise contacting the cancer cells with an endonuclease. The method for generating modified cancer cells may comprise expressing an endonuclease within the cancer cells. In some embodiments, a sequence encoding the endonuclease protein may be positioned within a vector, e.g., for viral delivery as discussed herein. The endonuclease may comprise a Cas9 or Cas12a endonuclease. Non-limiting examples of Cas9 or Cas12a endonucleases include, but are not limited to, S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpf1), Lachnospiraceae bacterium (LbCpf1), AsCpf1 RR variant, LbCpf1 RR variant, AsCpf1 RVR variant, C. jejuni Cas9 (CjCas9), N. meningitidis (NmCas9), S. thermophilus (StCas9), T. denticola (TdCas9), and Mad7. In some cases, the endonuclease does not comprise a Cas9 or Cas12a endonuclease.

In some instances, the endonuclease is a mutated endonuclease. In some embodiments, the mutated endonuclease comprises one or more mutation in nucleolytic domains and cannot cleave target DNA sequence but can still bind to the target DNA sequence, for instance, a dead Cas, or dCas. In some embodiments, the mutated endonuclease has reduced activity for targeted DNA cleavage. In some embodiments, the mutated endonuclease is derived from wildtype endonucleases such as Cas9, Cas12, Cas12a (or Cpf1 or Mad7), Cas12b (or C2c1 or Cpf2), Cas12c (C2c3), Cas12d (or CasY), Cas12e (or CasX), Cas13, Cas13a (or C2c2), Cas13b (or C2c6), Cas13c (or C2c7), Cas13d (or Casrx), Cas14, Cas14a, Cas14b, Cas14c, Casl, CaslB, Cas2, Cas3, Cas4, Cas5, Cas5e (CasD), Cas6, Cas6e, Cas6f, Cas7, Cas8a, Cas8a1, Cas8a2, Cas8b, Cas8c, Csnl, Csx12, Cas10, Cas10d, CasIO, CaslOd, CasF, CasG, CasH, Csy1, Csy2, Csy3, Csel (CasA), Cse2 (CasB), Cse3 (CasE), Cse4 (CasC), Csc1, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, CsxIO, Csx16, CsaX, Csx3, Csxl, Csx15, Csf1, Csf2, Csf3, Csf4, or Cul966, or derivative thereof, variant thereof, fragment thereof, or any combination thereof. In some embodiments, the mutated endonuclease is derived from the wildtype Cas9 or Cas12a family, which may include, but is not limited to, S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpf1), Lachnospiraceae bacterium (LbCpf1), AsCpf1 RR variant, LbCpf1 RR variant, AsCpf1 RVR variant, C. jejuni Cas9 (CjCas9), N. meningitidis (NmCas9), S. thermophilus (StCas9), T. denticola (TdCas9), and Mad7. In some instances, the mutated endonuclease is described herein in CRISPRi.

In some embodiments, the method for generating the plurality of modified cancer cells comprises an RNA interference (RNAi) gene silencing system. For instance, each gene modulatory reagent comprises a shRNA sequence targeting mRNA, or production of mRNA, wherein the mRNA encodes for a protein target from the library of protein targets. The shRNA may have homology to about 10 to about 50 contiguous nucleotides of the gene. The homology may be at least about 90% sequence identity. A sequence encoding the shRNA may be positioned within a vector, e.g., for viral delivery as discussed herein.

In some embodiments, the library of gene modulatory reagents comprises from about 10 to about 2,000, from about 10 to about 500, from about 10 to about 200, from about 10 to about 150, from about 50 to about 500, from about 50 to about 200, from about 50 to about 2,000, from about 100 to about 2,000, from about 500 to about 2,000, or from about 2000 to about 6,500 different gene modulatory reagents. In some embodiments, the library of gene modulatory reagents comprises about 405, 1210, 1615, 4830, or 6450 different gene modulatory reagents. In some cases, at least about 90% of the gene modulatory reagents are present in the library in a quantity within about 10% of the average gene modulatory reagent quantity.

In some embodiments, the library of protein targets comprises at least 1, at least 2, at least 3, at least 4, at least 5, at least 10, or at least 20 protein targets encoded by the genes of TABLE 9A. In some embodiments, the library of protein targets comprises at least 1, at least 2, at least 3, at least 4, at least 5, at least 10, or at least 20 protein targets encoded by the genes of TABLE 9B. In some embodiments, the library of protein targets comprises at least 1, at least 2, at least 3, at least 4, at least 5, at least 10, or at least 20 protein targets encoded by the kinase genes of TABLE 9C. In some embodiments, the library of protein targets comprises at least 1, at least 2, at least 3, at least 4, at least 5, at least 10, or at least 20 protein targets encoded by the IgSF genes of TABLE 9D.

In some embodiments, the sample of cancer cells comprises primary cancer cells. The sample of cancer cells may comprise about 10⁵ to about 10⁸ cells. The sample of cancer cells may comprise less than about 10⁸ cells. The sample of cancer cells may comprise less than about 10⁷ cells. The sample of cancer cells may comprise less than about 10⁶ cells. The sample of cancer cells may comprise less than about 10⁵ cells. The sample of cancer cells may have been processed to preserve cell viability. The method may thus further comprise preparing the sample of cancer cells to preserve cell viability prior to and/or after delivery of the library of gene modulatory reagents. The method may also further comprise propagating the modified cancer cells. Propagation may comprise maintenance of the modified cancer cells in a 2D in vitro culture. Propagation may comprise maintenance of the modified cancer cells in a 3D in vitro culture. Propagation may comprise maintenance of the modified cancer cells in vivo. In some cases, propagation occurs within an animal model, e.g., in a rodent.

### CRISPR-Based Methods

In some embodiments, a sample of cells is modified using a CRISPR-based method. The CRISPR-based method may comprise contacting the sample of cells with a plurality of gRNA sequences, wherein one or more of the gRNAs have sequence homology to a target gene encoding a protein targeted by a therapeutic agent. In some embodiments, sequence homology to a target gene comprises at least about 90% sequence identity of a continuous stretch of 10-50 nucleotides of the transcribed gRNA sequence to a homologous template strand of genomic DNA. In some embodiments, the template strand of genomic DNA is the sense strand corresponding to a targeted gene. In some embodiments, the template strand of genomic DNA is the antisense strand corresponding to a targeted gene. In some embodiments, contacting the sample of cells with the plurality of gRNA sequences comprises expressing the plurality of gRNA sequences in cells from the sample of cells using a CRISPR expression vector wherein the plurality of expressed gRNA sequences are expressed intracellularly.

Non-limiting examples of target genes are provided in TABLE 9A, TABLE 9B, TABLE 9C, and TABLE 9D. Non-limiting examples of therapeutic agents are provided in TABLES 3, 4, 10A, 10B, and 10C. In some embodiments, the sample of cells is contacted with at least one or a plurality of gRNA sequences chosen from SEQ ID NOS: 55-12949. In some embodiments, the sample of cells is contacted with at least one or a plurality of gRNA sequences, each having at least about 85% identity to a sequence chosen from SEQ ID NOS: 55-1669. In some embodiments, the sample of cells is contacted with at least one or a plurality of gRNA sequences chosen from SEQ ID NOS: 55-1669. In some embodiments, the sample of cells is contacted with at least one or a plurality of gRNA sequences, each having at least about 85% identity to a sequence chosen from SEQ ID NOS: 55-459. In some embodiments, the sample of cells is contacted with at least one or a plurality of gRNA sequences chosen from SEQ ID NOS: 55-459. In some embodiments, the sample of cells is contacted with at least one or a plurality of gRNA sequences, each having at least about 85% identity to a sequence chosen from SEQ ID NOS: 460-1669. In some embodiments, the sample of cells is contacted with at least one or a plurality of gRNA sequences chosen from SEQ ID NOS: 460-1669. In some embodiments, the sample of cells is contacted with at least one or a plurality of gRNA sequences, each having at least about 85% identity to a sequence chosen from SEQ ID NOS: 1670-8119. In some embodiments, the sample of cells is contacted with at least one or a plurality of gRNA sequences chosen from SEQ ID NOS: 1670-8119. In some embodiments, the sample of cells is contacted with at least one or a plurality of gRNA sequences, each having at least about 85% identity to a sequence chosen from SEQ ID NOS: 8120-12949. In some embodiments, the sample of cells is contacted with at least one or a plurality of gRNA sequences chosen from SEQ ID NOS: 8120-12949. The sample of cells is also contacted with an RNA-guided endonuclease, e.g., Cas9, Cas12, Cas12a (or Cpf1 or Mad7), Cas12b (or C2c1 or Cpf2), Cas12c (C2c3), Cas12d (or CasY), Cas12e (or CasX), Cas13, Cas13a (or C2c2), Cas13b (or C2c6), Cas13c (or C2c7), Cas13d (or Casrx), Cas14, Cas14a, Cas14b, Cas14c, Casl, CaslB, Cas2, Cas3, Cas4, Cas5, Cas5e (CasD), Cas6, Case, Casof, Cas7, Cas8a, Cas8a1, Cas8a2, Cas8b, Cas8c, Csnl, Csx12, Cas10, Cas10d, CasIO, CaslOd, CasF, CasG, CasH, Csy1, Csy2, Csy3, Csel (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, CsxIO, Csx16, CsaX, Csx3, Csx1, Csx15, Csf1, Csf2, Csf3, Csf4, Cul966, zinc finger nucleases (ZFN), transcription activator-like effector nucleases (TALEN), meganucleases, RNA-binding proteins (RBP), recombinases, flippases, transposases, Argonaute (Ago) proteins (e.g., prokaryotic Argonaute (pAgo), archaeal Argonaute (aAgo), or eukaryotic Argonaute (eAgo)), or derivative thereof, variant thereof, fragment thereof, or combination thereof. In some embodiments, contacting the cells from the sample of cells with the RNA-guided endonuclease comprises expressing an mRNA encoding the RNA-guided endonuclease in cells from the sample of cells, wherein the mRNA encoding the RNA-guided endonuclease is translated into the RNA-guided endonuclease by way of endogenous cellular protein translation mechanisms.

### RNAi and Other Non-CRISPRi Gene Repression

In some embodiments, a sample of cells is modified using an RNAi method. In some embodiments, the sample of cells is contacted with a plurality of shRNA sequences, each shRNA sequence complementary to a portion of a target mRNA of a protein targeted by a therapeutic agent. Non-limiting examples of target proteins include those encoded by the genes listed in TABLE 9A, TABLE 9B, TABLE 9C, and TABLE 9D. Non-limiting examples of therapeutic agents are provided in TABLES 3, 4, 10A, 10B, and 10C. In some embodiments, contacting the sample of cells with the plurality of shRNA sequences comprises expressing the plurality of shRNA sequences in cells from the sample of cells using an expression vector wherein the plurality of expressed shRNA sequences are expressed intracellularly. In some embodiments, contacting the sample of cells with the plurality of shRNA sequences comprises transfection of shRNA sequences.

In some embodiments, a sample of cells is modified using an oligonucleotide to repress expression of the target. In some embodiments, the oligonucleotide comprises antisense oligonucleotides (ASOs), short interfering RNAs (siRNAs), aptamers and microRNA (miRNA) inhibitors (anti-miRs) and mimics (pro-miRs).

In some instances, a sample of cells is modified using a transcription activator-like effector nucleases (TALEN) to repress expression of the target.

In some instances, a sample of cells is modified using a zinc finger protein with a repressor domain to repress expression of the target.

### CRISPR Interference (CRISPRi)

CRISPRi is a genetic modulation technique that allows sequence-specific repression of gene expression using gRNA to block transcription of targeted mRNA without altering genomic gene sequences. CRISPRi can repress transcription by blocking either transcriptional initiation or elongation. CRISPRi can repress transcription to knock-down a function of a targeted gene or a plurality of targeted genes.

In one aspect, provided is a library comprising a plurality of gene modulatory reagents, wherein each modulatory reagent comprises a guide RNA (gRNA) homologous to a target gene. Non-limiting examples of gRNA sequences used in CRISPRi are provided in TABLE 9A, TABLE 9B, TABLE 9C, and TABLE 9D. Guide RNA sequences listed in TABLE 9A, TABLE 9B, TABLE 9C, and TABLE 9D correspond to DNA sequences indicated within or adjacent to a respective target gene. Guide RNA sequences may be represented in a corresponding DNA sequence in an expression vector. Guide RNA sequences listed in TABLE 9A, TABLE 9B, TABLE 9C, and TABLE 9D when present in a cell as part of a gRNA correspond to the equivalent transcribed RNA sequence compared to the DNA sequences listed in TABLE 9A, TABLE 9B, TABLE 9C, and TABLE 9D. Non-limiting examples of gRNA sequences used in CRISPRi include sequences at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 55-12949. In some embodiments, gRNA sequences used in CRISPRi are constructed to target one or more genes encoding a serine/threonine kinase. In some embodiments, gRNA sequences used in CRISPRi are constructed to target one or more genes encoding a tyrosine kinase. In some embodiments, gRNA sequences used in CRISPRi are constructed to target one or more genes encoding IgSF family members.

In some embodiments, the library comprises an RNA-guided endonuclease that does not have nuclease activity and therefore does not cleave or edit the genome. In some embodiments, the library comprises an RNA-guided endonuclease that comprises reduced nuclease activity and therefore cleaves or edits the genome with a reduced capacity compared to an endonuclease that has not been mutated. In some embodiments, the RNA-guided endonuclease is a mutated endonuclease. In some embodiments, the mutated endonuclease comprises at least one mutation in a nucleolytic domain and cannot cleave target DNA sequence but can still bind to the target DNA sequence. In some embodiments, the mutated endonuclease is mutated from a wildtype endonucleases such as Cas9, Cas12, Cas12a (or Cpf1 or Mad7), Cas12b (or C2c1 or Cpf2), Cas12c (C2c3), Cas12d (or CasY), Cas12e (or CasX), Cas13, Cas13a (or C2c2), Cas13b (or C2c6), Cas13c (or C2c7), Cas13d (or Casrx), Cas14, Cas14a, Cas14b, Cas14c, Casl, CaslB, Cas2, Cas3, Cas4, Cas5, Cas5e (CasD), Cas6, Cas6e, Casof, Cas7, Cas8a, Cas8a1, Cas8a2, Cas8b, Cas8c, Csnl, Csx12, Cas10, Cas10d, CasIO, CaslOd, CasF, CasG, CasH, Csy1, Csy2, Csy3, Csel (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, CsxIO, Csx16, CsaX, Csx3, Csx1, Csx15, Csf1, Csf2, Csf3, Csf4, or Cul966, or derivative thereof, variant thereof, fragment thereof, or any combination thereof. In some embodiments, the mutated endonuclease is mutated from the wildtype Cas9 or Cas12a family, which may include, but is not limited to, S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpf1), Lachnospiraceae bacterium (LbCpf1), AsCpf1 RR variant, LbCpf1 RR variant, AsCpf1 RVR variant, C. jejuni Cas9 (CjCas9), N. meningitidis (NmCas9), S. thermophilus (StCas9), T. denticola (TdCas9), and Mad7.

A nuclease-inactive Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., Science. 337:816-821 (2012); Qi et al., (2013) Cell. 28; 152 (5): 1173-83). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821 (2012); Qi et al., Cell. 28; 152 (5): 1173-83 (2013). Additional exemplary suitable nuclease-inactive Cas9 domains include, but are not limited to, D10A/H840A, D10A/D839A/H840A, and D10A/D839A/H840A/N863A mutant domains (See, e.g., Prashant et al., Nature Biotechnology. 2013; 31 (9): 833-838).

In some embodiments, a Cas nuclease is provided in a split system. For instance, a Cas nuclease is “split” into an N-terminal portion and a C-terminal portion. The portions may be delivered on separate vectors (e.g., separate lentiviral vectors) into one cell and co-expressed, may be joined to form a complete and functional Cas protein (e.g., via splicing).

In some embodiments, CRISPRi components comprises a catalytically inactive Cas nuclease (dCas). In some embodiments, the mutated endonuclease comprises a catalytically inactivate Cas nuclease (dCas). In some embodiments, the dCas comprises a mutated Cas protein. In some embodiments, the dCas is expressed from an expression vector (e.g., a lentiviral vector) that has been introduced into cells from a cell sample. In some embodiments, the mutated Cas protein is a mutated Cas9 protein. In some embodiments, the mutated Cas9 protein is a dCas9 protein. In some embodiments, the mutated Cas protein is a mutated Cas12a protein. In some embodiments, the mutated Cas12a protein is a dCas12a protein. In some embodiments, the mutated Cas protein is a mutated Cas12f protein. In some embodiments, the mutated Cas12f protein is a dCas12f protein. Exemplary dCas protein sequences are listed in TABLE 11. In some embodiments, the dCas is a truncated Cas protein. In some embodiments, the dCas9 comprises a sequence at least 90% identical to the dCas9 of SEQ ID NO: 12950 (S. pyogenes dCas9 (D10A and H840A)). In some cases, the dCas9 has a sequence at least about 85%, 86%, 87%, 88%, 89%, 90%, 91%, 92%, 93%, 94%, 95%, 96%, 97%, 98%, or 99% identical to the dCas of SEQ ID NO: 12950. In some cases, the sequence of dCas9 is SEQ ID NO: 12950. In some embodiments, the dCas 12a comprises a sequence at least 90% identical to the dCas12a of SEQ ID NO: 12951. In some cases, the dCas12a has a sequence at least about 85%, 86%, 87%, 88%, 89%, 90%, 91%, 92%, 93%, 94%, 95%, 96%, 97%, 98%, or 99% identical to the dCas of SEQ ID NO: 12951. In some cases, the sequence of dCas12a is SEQ ID NO: 12951. In some embodiments, the dCas12f1 comprises a sequence at least 90% identical to the dCas12f1 of SEQ ID NO: 12952. In some cases, the dCas12f1 has a sequence at least about 85%, 86%, 87%, 88%, 89%, 90%, 91%, 92%, 93%, 94%, 95%, 96%, 97%, 98%, or 99% identical to the dCas of SEQ ID NO: 12952. In some cases, the sequence of dCas12f1 is SEQ ID NO: 12952. In some cases, the dCas12f1 is termed dCasMINI. In some embodiments, the dCasMINI comprises a sequence at least 90% identical to the dCas12f1 of SEQ ID NO: 12953. In some cases, the dCasMINI has a sequence at least about 85%, 86%, 87%, 88%, 89%, 90%, 91%, 92%, 93%, 94%, 95%, 96%, 97%, 98%, or 99% identical to the dCas of SEQ ID NO: 12953. In some cases, the sequence of dCasMINI is SEQ ID NO: 12953.

In some embodiments, the dCas9 is a truncated or mutated Cas9 protein. In some embodiments, the dCas comprises dCas12. In some embodiments, the dCas12 is a truncated or mutated Cas12 protein. In some embodiments, the dCas comprises a dCas9 from Streptococcus pyogenes, Staphylococcus aureus, Campylobacter jejuni, S. thermophilus, S. pneumoniae, Neisseria meningitidis, Corynebacter diphtheriae, Eubacterium ventriosum, Streptococcus pasteurianus, Lactobacillus farciminis, Sphaerochaeta globus, Azospirillum B510, Gluconacetobacter diazotrophicus, Neisseria cinerea, Roseburia intestinalis, Parvibaculum lavamentivorans, Nitratifractor salsuginis DSM 16511, Campylobacter lari CF89-12, or Streptococcus thermophilus LMD-9. In some embodiments, the dCas has a REC2 domain deletion. In some embodiments, the dCas has a REC3 domain deletion. In some embodiments, the dCas has a HNH deletion. In some embodiments, the dCas has a nuclease (NUC) lobe deletion. In some embodiments, the dCas has a RuvC deletion. These truncations include but are not limited to, REC2 domain deletion, REC3 domain deletion, HNH deletion, and deletions of the domains of the nuclease (NUC) lobe, RuvC, or any combination of the aforementioned domains.

In some embodiments, the dCas is a dCas9. In some embodiments, the dCas9 has one or more mutation in RuvC domain. In some embodiments, the dCas9 has one or more mutation in HNH domain. In some embodiments, the dCas9 has a point mutation D10A in RuvC domain. In some embodiments, the dCas9 has a point mutation H840A in HNH domain. In some embodiments, the dCas9 has point mutations at both D10A and H840A in RuvC and HNH domains, respectively.

In some embodiments, the CRISPRi components comprise a modulatory domain, such as a repressor domain. In some embodiments, nucleotide sequence in a vector encodes CRISPRi components comprising a modulatory domain, such as a repressor domain. In some embodiments, nucleotide sequence encoding the repressor domain is fused to the 5′ end of nucleotide sequence encoding the endonuclease. In some embodiments, the nucleotide sequence encoding the repressor domain is fused to the 5′ end of nucleotide sequence encoding the endonuclease via nucleotide sequencing encoding at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids comprising a linker, where the linker may be flexible. In some embodiments, the repressor domain is fused to the N-terminus of the endonuclease via at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids comprising a linker where the linker may be a flexible linker. In some embodiments, nucleotide sequencing encoding the repressor domain is fused to the 5′ end of nucleotide sequence encoding the endonuclease via 16 amino acid flexible linker. In some embodiments, the repressor domain is fused to the N-terminus of the endonuclease via a flexible linker comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 amino acids. In some embodiments, the repressor domain is fused to the N-terminus of the endonuclease via a flexible linker comprising the amino acid sequence YPTFLYKVVGGSGGSA or YPTFLYKVVGGSGGSA with 1, 2, 3, 4, 5, 6, 7, 8, 9, or 10 amino acid substitutions, additions, deletions, or combinations thereof. In some embodiments, the repressor domain is fused to the N-terminus of the endonuclease via a 16 amino acid flexible linker. In some embodiments, the repressor domain is fused to the N-terminus of the endonuclease via a flexible linker comprising the amino acid sequence YPTFLYKVVGGSGGSA. In some embodiments, the amino acid flexible linker sequence comprises SEQ ID NO: 12954. In some embodiments, the repressor domain is fused to the N-terminus of the endonuclease via a flexible linker consisting of the amino acid sequence YPTFLYKVVGGSGGSA. An exemplary linker peptide sequence is listed in TABLE 13. In some embodiments, nucleotide sequence encoding the repressor domain is fused to the 5′ end of nucleotide sequence encoding the endonuclease with intervening nucleotide sequence encoding a linker domain, wherein the nucleotide sequences encodes a single polypeptide chain comprising a repressor domain, a linker domain, and an endonuclease. In some embodiments, the endonuclease is a dCas protein.

In some embodiments, the repressor domain comprises KRAB (also referred to as KOX), SID, MBD2, MBD3, HP1a, DNMT family (including DNMT1, DNMT3A, DNMT3B, DNMT3L, DNMT2A), Sin3a, Rb, MeCP2, ROM2, AtHD2A, LSD1, SUV39H1, G9a (EHMT2), ZFP1, ZFP14, ZFP82, ZIM2, ZIM3, ZNF18, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF419, ZNF140, ZNF175, ZNF214, ZNF184, ZNF8, ZNF60, ZNF595, ZNF596, ZNF10, ZNF37A, ZNF394, ZNF610, ZNF273, ZNF34, ZNF250, ZNF98, ZNF675, ZNF213, ZFP28-2, ZNF224, ZNF257, GLP (EHMT2), or a variant or combination thereof. In some embodiments, the repressor domain is selected from TABLE 1. In some embodiments, the repressor domain comprises a KRAB domain comprising an amino acid sequence listed in the KRAB domain coordinates for exemplary human KRAB domain proteins listed in TABLE 1. In some embodiments, the repressor domain comprises KRAB domain from ZNF10 comprising amino acids 13-85 corresponding to KRAB domain sequence from Uniprot ID Entry P21506. In some embodiments, the repressor domain comprises KRAB domain from ZNF10 comprising amino acids 14-75 of NCBI protein entry NP_056209.2 corresponding to KRAB domain sequence. In some embodiments, the repressor domain comprises KRAB domain from ZNF10 comprising amino acids 14-75 of NCBI protein entry NP_056209.2 with an L38V substitution corresponding to KRAB domain sequence. In some embodiments, the repressor domain is selected from TABLE 2. In some embodiments, the repressor domain comprises a KRAB domain comprising an amino acid sequence listed in the KRAB domain coordinates for exemplary human KRAB domain proteins listed in TABLE 2. In some embodiments, the repressor domain comprises KRAB domain comprising amino acid sequence of KRAB domain coordinates of ZFP1 from SEQ ID NO: 1. In some embodiments, the repressor domain comprises KRAB domain comprising amino acid sequence of KRAB domain coordinates of ZFP14 from SEQ ID NO: 2. In some embodiments, the repressor domain comprises KRAB domain comprising amino acid sequence of KRAB domain coordinates of ZFP82 from SEQ ID NO: 3. In some embodiments, the repressor domain comprises KRAB domain comprising amino acid sequence of KRAB domain coordinates of ZIM2 from SEQ ID NO: 4. In some embodiments, the repressor domain comprises KRAB domain comprising amino acid sequence of KRAB domain coordinates of ZIM3 from SEQ ID NO: 5. In some embodiments, the repressor domain comprises KRAB domain comprising amino acid sequence of KRAB domain coordinates of ZNF140 from SEQ ID NO: 6. In some embodiments, the repressor domain comprises KRAB domain comprising amino acid sequence of KRAB domain coordinates of ZNF175 from SEQ ID NO: 7. In some embodiments, the repressor domain comprises KRAB domain comprising amino acid sequence of KRAB domain coordinates of ZNF18 from SEQ ID NO: 8. In some embodiments, the repressor domain comprises KRAB domain comprising amino acid sequence of KRAB domain coordinates of ZNF250 from SEQ ID NO: 14. In some embodiments, the repressor domain comprises KRAB domain comprising amino acid sequence of KRAB domain coordinates of ZNF37A from SEQ ID NO: 27. In some embodiments, the repressor domain comprises KRAB domain comprising amino acid sequence of KRAB domain coordinates of ZNF419 from SEQ ID NO: 31. In some embodiments, the repressor domain comprises KRAB domain comprising amino acid sequence of KRAB domain coordinates of ZNF595 from SEQ ID NO: 42. In some embodiments, the repressor domain comprises KRAB domain comprising amino acid sequence of KRAB domain coordinates of ZNF60 from SEQ ID NO: 44. In some embodiments, the repressor domain comprises KRAB domain comprising amino acid sequence of KRAB domain coordinates of ZNF8 from SEQ ID NO: 52. In some embodiments, the repressor domain comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the KRAB domain coordinates listed in TABLE 2 for any one of SEQ ID NOs: 1-54. In some embodiments, the repressor domain comprises a sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 1-54. In some embodiments, the repressor domain comprises amino acid sequence identical to KRAB domain coordinates listed in TABLE 2 for any one of SEQ ID NOs: 1-54. In some embodiments, the repressor domain comprises amino acid sequence identical to a continuous portion of a human KRAB domain protein listed in TABLE 2, wherein the continuous portion comprises at least about 40, 50, 60, 70, or 80 amino acids identical to a segment of KRAB domain coordinates listed in TABLE 2 from SEQ ID NOs: 1-54. In some embodiments, the repressor domain comprises an amino acid sequence at least about 40, 50, 60, 70, 80, 90, or 100 amino acids in length, wherein the amino acid sequence is at least about 95%, 96%, 97%, 98%, 99%, or 100% identical to a portion of human KRAB domain protein listed in TABLE 2, wherein the sequence of the portion of human KRAB domain protein is contained within one of SEQ ID NOs: 1-54. In some embodiments, the repressor domain comprises an amino acid sequence less than about 150, 140, 130, 120, 110, 100, 90, or 80 amino acids in length, wherein the amino acid sequence is at least about 95%, 96%, 97%, 98%, 99%, or 100% identical to a portion of human KRAB domain protein listed in TABLE 2, wherein the sequence of the portion of human KRAB domain protein is contained within one of SEQ ID NOs: 1-54. In some embodiments, the repressor domain comprises about 110 amino acids of a KRAB domain protein listed in TABLE 2. In some embodiments, the repressor domain comprises about 100 amino acids of a KRAB domain protein listed in TABLE 2. In some embodiments, the repressor domain comprises about 90 amino acids of a KRAB domain protein listed in TABLE 2. In some embodiments, the repressor domain comprises about 80 amino acids of a KRAB domain protein listed in TABLE 2. In some embodiments, the repressor domain comprises about 70 amino acids of a KRAB domain protein listed in TABLE 2.

Nuclear localization signals (NLS) are short peptides that act as a signal fragment that mediates the transport of proteins from the cytoplasm into the nucleus. In some embodiments, nucleotide sequencing encoding a nuclear localization signal (NLS) is fused near or to the 5′ end of the nucleotide sequence encoding the endonuclease. In some embodiments, nucleotide sequencing encoding a nuclear localization signal (NLS) is fused near or to the 3′ end of the nucleotide sequence encoding the repressor domain. In some embodiments, the nuclear localization signal is fused near or to N-terminus of the endonuclease. In some embodiments, the nuclear localization signal is fused to the N-terminus of the endonuclease. In some embodiments, the nuclear localization signal is fused near or to the C-terminus of the repressor domain. In some embodiments, the nuclear localization signal is fused to the C-terminus of the repressor domain. In some embodiments, at least 1, at least 2, at least 3, at least 4, or at least 5 copies of nucleotide sequence encoding nuclear localization signals are fused near or to the 5′ end of the nucleotide sequence encoding the endonuclease. In some embodiments, at least 1, at least 2, at least 3, at least 4, or at least 5 copies of nucleotide sequence encoding nuclear localization signals are fused near or to the N-terminus of the endonuclease. In some embodiments, at least 1, at least 2, at least 3, at least 4, or at least 5 copies of nucleotide sequence encoding nuclear localization signals are fused near or to the 3′ end of the nucleotide sequence encoding the repressor domain. In some embodiments, at least 1, at least 2, at least 3, at least 4, or at least 5 copies of nucleotide sequence encoding nuclear localization signals are fused near or to the C-terminus of the repressor domain.

In some embodiments, the nuclear localization signal comprises a classical nuclear localization signal, non-classical NLS signal, or other types. In some embodiments, the nuclear localization comprises nuclear localization sequences from simian virus 40 (SV40), VACM-1/CUL5, CXCR4, VP1, 53BP1, ING4, IER5, ERK5, Hrp1, UL79, EWS, PTHrP, Pho4, rpL23a, MSX1, NLS-RARa, or as described in Lu, J. et al. 2021. Cell Communication and Signaling, which is incorporated by reference in its entity. In some embodiments, nucleotide sequence encoding an SV40 NLS comprising the amino acid sequence of SEQ ID NO: 12955 is fused near or to the 5′ end of the nucleotide sequence encoding the endonuclease. In some embodiments, an SV40 NLS comprising the amino acid sequence of SEQ ID NO: 12955 is fused near or to the N-terminus of the endonuclease. In some embodiments, nucleotide sequence encoding an SV40 NLS comprising the amino acid sequence of SEQ ID NO: 12956 is fused near or to the 5′ end of the nucleotide sequence encoding the endonuclease. In some embodiments, an SV40 NLS comprising the amino acid sequence of SEQ ID NO: 12956 is fused near or to the amino-terminal end of the endonuclease. In some embodiments, nucleotide sequence encoding an SV40 NLS comprising the amino acid sequence of SEQ ID NO: 12957 is fused near or to the 5′ end of the nucleotide sequence encoding the endonuclease. In some embodiments, an SV40 NLS comprising the amino acid sequence of SEQ ID NO: 12957 is fused near or to the amino-terminal end of the endonuclease. In some embodiments, nucleotide sequence encoding a triple SV40 NLS of SEQ ID NO: 12958 is fused near or to the 5′ end of the nucleotide sequence encoding the endonuclease. In some embodiments, nucleotide sequence encoding triple SV40 NLS of SEQ ID NO: 12958 is fused to the 5′ end of the nucleotide sequence encoding the endonuclease. In some embodiments, a triple SV40 NLS of SEQ ID NO: 12958 is fused near or to the N-terminus of the endonuclease. In some embodiments, a triple SV40 NLS of SEQ ID NO: 12958 is fused to the N-terminus of the endonuclease. In some embodiments, nucleotide sequence encoding the nuclear localization signal fused near or to the 5′ end of the nucleotide sequence encoding the endonuclease comprises amino acid sequence of nucleoplasmin NLS of SEQ ID NO: 12959. In some embodiments, the nuclear localization signal fused to the N-terminus of the endonuclease comprises nucleoplasmin NLS of SEQ ID NO: 12959. In some embodiments, nucleotide sequence encoding a single nucleoplasmin NLS of SEQ ID NO: 12959 is fused near or to the 5′ end of the nucleotide sequence encoding the endonuclease. In some embodiments, a single nucleoplasmin NLS of SEQ ID NO: 12959 is fused to the N-terminus of the endonuclease. Exemplary NLS peptide sequences are listed in TABLE 13. In some embodiments, nucleotide sequence encoding the NLS comprises SEQ ID NO: 12981. In some embodiments, nucleotide sequence encoding the NLS is incorporated into a vector sequence (e.g., a CRISPRi vector sequence) that is used in a gene modulation system described herein.

In some embodiments, nucleotide sequence encoding a nuclear localization signal (NLS) is fused near or to the 3′ end of the nucleotide sequence encoding the endonuclease. In some embodiments, the nuclear localization signal is fused near or to C-terminus of the endonuclease. In some embodiments, at least 1, at least 2, at least 3, at least 4, or at least 5 copies of nucleotide sequence encoding nuclear localization signals are fused near or to the 3′ end of the nucleotide sequence encoding the endonuclease. In some embodiments, at least 1, at least 2, at least 3, at least 4, or at least 5 copies of nuclear localization signal amino acid sequences are fused to the C-terminus of the endonuclease. In some embodiments, nucleotide sequence encoding the nuclear localization signal fused near or to the 3′ end of the nucleotide sequence encoding the endonuclease comprises amino acid sequence of nucleoplasmin NLS of SEQ ID NO: 12959. In some embodiments, the nuclear localization signal fused to the C-terminus of the endonuclease comprises nucleoplasmin NLS of SEQ ID NO: 12959. In some embodiments, nucleotide sequence encoding a single nucleoplasmin NLS of SEQ ID NO: 12959 is fused near or to the 3′ end of the nucleotide sequence encoding the endonuclease. In some embodiments, nucleotide sequence encoding a single nucleoplasmin NLS of SEQ ID NO: 12959 is fused to the 3′ end of the nucleotide sequence encoding the endonuclease. In some embodiments, a single nucleoplasmin NLS of SEQ ID NO: 12959 is fused to the C-terminus of the endonuclease. In some embodiments, nucleotide sequence encoding an SV40 NLS comprising the amino acid sequence of SEQ ID NO: 12955, 12956, 12957, or 12958 is fused near or to the 3′ end of the nucleotide sequence encoding the endonuclease. In some embodiments, nucleotide sequence encoding triple SV40 NLS of SEQ ID NO: 12958 is fused to the 3′ end of the nucleotide sequence encoding the endonuclease. In some embodiments, a triple SV40 NLS of SEQ ID NO: 12958 is fused to the C-terminus of the endonuclease. In some embodiments, nucleotide sequence encoding the amino acid sequence Gly Ser immediately precedes the nucleotide sequence encoding the nucleoplasmin NLS of SEQ ID NO: 12959. In some embodiments, nucleotide sequence encoding the amino acid sequence Gly Ser immediately follows the nucleotide sequence encoding the nucleoplasmin NLS of SEQ ID NO: 12959. In some embodiments, nucleotide sequence encoding T2A follows a nucleotide sequence encoding the nucleoplasmin NLS of SEQ ID NO: 12959. In some embodiments, nucleotide sequence encoding T2A follows nucleotide sequence encoding the amino acid sequence Gly Ser which immediately follows nucleotide sequence encoding the nucleoplasmin NLS of SEQ ID NO: 12959. In some embodiments, nucleotide sequence encoding T2A follows nucleotide sequence encoding the nucleoplasmin NLS of SEQ ID NO: 12959 which follows nucleotide sequence encoding the amino acid sequence Gly Ser. Exemplary NLS and T2A peptide sequences are listed in TABLE 13. In some embodiments, nucleotide sequence encoding the T2A peptide comprises SEQ ID NO: 12971. In some embodiments, nucleotide sequence encoding the T2A peptide with linkers comprises SEQ ID NO: 12972.

In some embodiments, a 2A self-cleaving peptide is fused in order to link a downstream reporter to the endonuclease. In some embodiments, nucleotide sequence encoding the 2A self-cleaving peptide is fused near or to nucleotide sequencing encoding the 3′ end of the nuclear localization signal located near or on the 3′ end of the nucleotide sequence encoding the endonuclease. In some embodiments, the 2A self-cleaving peptide is fused near or to the C-terminus of the nuclear localization signal located near or on the C-terminus of the endonuclease. In some embodiments, the 2A self-cleaving peptide comprises P2A, E2A, F2A, or T2A. In some embodiments, the 2A self-cleaving peptide comprises T2A of SEQ ID NO: 12960. In some embodiments, the 2A self-cleaving peptide is fused near or to the N-terminal peptide sequence of the nucleoplasmin NLS. In some embodiments, the peptide sequence of the nucleoplasmin NLS immediately precedes the peptide sequence of the 2A self-cleaving peptide. In some embodiments, the peptide sequence of the nucleoplasmin NLS precedes the peptide sequence of the 2A self-cleaving peptide with an insertion of 1, 2, 3, 4, 5, 6, 7, 8, 9, or 10 amino acid residues in between the peptide sequence of the nucleoplasmin NLS and the peptide sequence of the 2A self-cleaving peptide.

In some embodiments, the 2A self-cleaving peptide is fused near or to the C-terminal peptide sequence of the nucleoplasmin NLS. In some embodiments, the downstream reporter comprises Thy-1 cell surface antigen (also known as CD90). In some embodiments, nucleotide sequence encoding the Thy-1 marker comprises SEQ ID NO: 12973.

In some instances, the knocking down of the function of the target gene comprises use of CRISPRi to repress expression of the target gene. In some embodiments, the knocking down of the function of the target gene comprises use of CRISPRi to repress a function of the target gene below a threshold level of function. In some embodiments, the knocking down of the function of the target gene comprises use of CRISPRi to repress mRNA expression of a target gene below a threshold level mRNA expression. In some embodiments, the threshold level of mRNA expression is less than about 50%, 40%, 30%, 20%, 15%, 10%, 5%, 3%, 2%, or 1% of the mRNA expression level in cells that are not targeted to knock down their function.

### Plasmid Vectors for CRISPRi

In some embodiments, a plasmid vector is used for expression of a Cas enzyme and a sgRNA specific for the target gene or target genes. In some embodiments, the plasmid vector used for expression of the Cas enzyme and the sgRNA is contained within a single vector. In some embodiments, the plasmid vector is used to produce a lentiviral delivery vector to delivery an ssRNA copy of the vector sequence to cells of the cell sample. In some embodiments, the ssRNA copy of the vector sequence is converted into dsDNA and integrated into the genome of cells of the cell sample. In some embodiments, integrated dsDNA comprising the vector sequence is configured to transcribe an sgRNA and a mutated endonuclease (e.g., a dCas) fused to a transcription repressor domain (e.g., a KRAB domain). In some embodiments, integrated dsDNA comprising the vector sequence is configured to separately transcribe an sgRNA from a first promoter and a mutated endonuclease (e.g., a dCas) fused to a transcription repressor domain (e.g., a KRAB domain) from a second promoter. In some embodiments, the first promoter comprises a U6 promoter. In some embodiments, the second promoter comprises a UbiC promoter. In some embodiments, the first and second promoter drive ubiquitous expression in a variety of cell types. In some embodiments, one or more promoter sequences are contained within the plasmid vector to promote transcription of elements contained within the plasmid vector. In some embodiments, the one or more promoter sequences may be selected from RSV, U6, UbiC, Thy1, AmpR, CMV, SV40, EF1α, and β-actin. In some embodiments, the plasmid vector contains several long terminal repeats (LTR). In some embodiments, the plasmid vector contains two identical or nearly identical LTR sequences. In some embodiments, two identical LTR sequences are positioned flanking sequences in the plasmid vector containing DNA coding for gRNA and Cas enzyme and sequences which promote expression of the gRNA and Cas enzyme and sequences which further help to define the function of the gRNA and the Cas enzyme. In some embodiments, an LTR sequence positioned 5′ relative to the gRNA and Cas enzyme sequences is termed the 5′ LTR and expression of the 5′ LTR is regulated by an upstream promoter sequence. In some embodiments, the upstream promoter sequence for the 5′ LTR is an RSV promoter sequence. In some embodiments, the RSV promoter is used to produce lentiviral vectors. In some embodiments, the nucleotide sequence for the RSV promoter comprises SEQ ID NO: 12961. In some embodiments, downstream from the 5′ LTR in the plasmid vector is a sgRNA sequence specific for the target gene or target genes and a trans-activating crispr RNA (tracrRNA). In some embodiments, the nucleotide sequence for a truncated 5′ LTR comprises SEQ ID NO: 12962. The tracrRNA is partially complementary to and base pairs with a continuous transcript (pre-crRNA) forming an RNA duplex. In some embodiments, sgRNA sequence specific for the target gene or target genes and a tracrRNA has a promoter sequence located upstream to promote expression of the sgRNA and tracrRNA. In some embodiments, the promoter sequence located upstream to promote expression of the sgRNA and tracrRNA is a U6 promoter sequence. In some embodiments, the U6 promoter sequence comprises a minimum human U6 promoter sequence of SEQ ID NO: 12964. In some embodiments, the U6 promoter sequence comprises a full length human U6 promoter sequence of SEQ ID NO: 12963. In some embodiments, the promoter sequence located upstream to promote expression of the sgRNA and tracrRNA is a minimal compound pol-III promoter M11 sequence. In some embodiments, the minimal compound pol-III promoter M11 sequence comprises SEQ ID NO: 12965. In some embodiments, a 3′ LTR sequence is positioned in the plasmid vector to produce lentiviral vector with ssRNA vector sequence corresponding to the nucleotide sequence in between a truncated 5′ LTR and the 3′ LTR. In some embodiments, the nucleotide sequence for the 3′ LTR sequence comprises SEQ ID NO: 12974. In some embodiments, the plasmid vector comprises an SV40 polyadenylation signal sequence comprising SEQ ID NO: 12975. In some embodiment, the plasmid comprises an Ori sequence comprising SEQ ID NO: 12976. In some embodiment, the plasmid comprises a sequence encoding AmpR comprising SEQ ID NO: 12977. In some embodiment, the plasmid comprises a sequence encoding an AmpR promoter to drive AmpR expression comprising SEQ ID NO: 12977.

In some embodiments, sequence encoding a Cas enzyme is located downstream of the sgRNA and tracrRNA sequences. In some embodiments, a promoter sequence is located upstream of the Cas enzyme sequence to promote expression of the Cas enzyme. In some embodiments, the promoter sequence located upstream of the Cas enzyme sequence to promote expression of the Cas enzyme is a UbiC promoter sequence. In some embodiments, the UbiC promoter sequence comprises SEQ ID NO: 12970. In some embodiments, an epigenetic modulator sequence is located upstream of the Cas enzyme sequence and downstream of the promoter sequence located upstream of the Cas enzyme sequence to promote expression of the Cas enzyme. In some embodiments, the epigenetic modulator sequence is arranged in the vector to code for an epigenetic modulator protein sequence in frame with the Cas enzyme and capable of being transcribed from a single transcript. In some embodiments, the epigenetic modulator sequence codes for an epigenetic modulator or functional fragment thereof are selected from TABLE 1 or TABLE 2. In some embodiments, the epigenetic modulator sequence codes for an epigenetic modulator that functions to repress transcription. In some embodiments, the epigenetic modulator sequence that codes for the epigenetic modulator that functions to repress transcription is capable of repressing transcription of the target gene or target genes of interest which contain sequence complimentary to the sgRNA sequence. In some embodiments, the epigenetic modulator sequence that codes for the epigenetic modulator that functions to repress transcription is capable of knocking down the function of the target gene or target genes of interest which contain sequence complimentary to the sgRNA sequence by repressing transcription of the target gene or target genes of interest. In some embodiments, the epigenetic modulator sequences codes for a KRAB domain. In some embodiments, the KRAB domain is a ZNF10 KRAB domain. In some embodiments, the KRAB domain is a ZFP1 KRAB domain. In some embodiments, the KRAB domain is a ZFP14 KRAB domain. In some embodiments, the KRAB domain is a ZFP82 KRAB domain. In some embodiments, the KRAB domain is a ZIM2 KRAB domain. In some embodiments, the KRAB domain is a ZIM3 KRAB domain. In some embodiments, the KRAB domain is a ZNF140 KRAB domain. In some embodiments, the KRAB domain is a ZNF175 KRAB domain. In some embodiments, the KRAB domain is a ZNF18 KRAB domain. In some embodiments, the KRAB domain is a ZNF250 KRAB domain. In some embodiments, the KRAB domain is a ZNF37A KRAB domain. In some embodiments, the KRAB domain is a ZNF419 KRAB domain. In some embodiments, the KRAB domain is a ZNF595 KRAB domain. In some embodiments, the KRAB domain is a ZNF60 KRAB domain. In some embodiments, the KRAB domain is a ZNF8 KRAB domain.

In some embodiments, a linker sequence is positioned between the epigenetic modulator sequence and the Cas enzyme sequence. In some embodiments, the linker sequence retains the same coding frame for the epigenetic modulator sequence and the Cas enzyme sequence to be transcribed within a single transcript. In some embodiments, the linker sequence comprises a sequence coding for between 1-100 amino acids. In some embodiments, one or more protein domain sequences are positioned in between the epigenetic modulator sequence and the Cas enzyme sequence. In some embodiments, the one or more protein domain sequences prevent against steric hindrance between the epigenetic modulator and the Cas enzyme which are located together in a translated fusion protein sequence. In some embodiments, an NLS sequence is positioned in between the linker sequence and the Cas enzyme sequence. In some embodiments, the NLS sequence positioned in between the linker sequence and the Cas enzyme sequence is an SV40 NLS sequence. In some embodiments, the SV40 NLS comprises SEQ ID NO: 12981. In some embodiments, the NLS sequence positioned in between the linker sequence and the Cas enzyme sequence functions to translocate the translated fusion protein sequence comprising an epigenetic modulator and a Cas enzyme from cytoplasm to the nucleus. In some embodiments, the NLS sequence positioned in between the linker sequence and the Cas enzyme sequence allows the epigenetic modulator and a Cas enzyme fusion protein to repress transcription from a genomic locus corresponding to the sgRNA sequence or sgRNA sequences expressed from the plasmid vector. In some embodiments, a transactivation domain 2 (TA2) motif is positioned downstream of the Cas enzyme sequence coding for a TA2 motif which can be transcribed in frame with the Cas enzyme sequence. In some embodiments, an NLS sequence is positioned downstream of the Cas enzyme sequence. In some embodiments, the NLS sequence positioned downstream of the Cas enzyme sequence is a nucleoplasmin NLS sequence. In some embodiments, the NLS sequence positioned downstream of the Cas enzyme sequence functions to translocate the translated fusion protein sequence comprising an epigenetic modulator and a Cas enzyme from cytoplasm to the nucleus. In some embodiments, an LTR named a 3′ LTR which is identical to at least a portion of the 5′ LTR sequence is positioned downstream of the Cas9 enzyme sequence. In some embodiments, contained in between the 5′ LTR sequence and the 3′ LTR sequence are sequencing comprising a U6 promoter, a gRNA sequence, a tracrRNA sequence, a UbiC promoter, an epigenetic modulator sequence, a linker sequence, an SV40 NLS sequence, a Cas enzyme sequence, a nucleoplasmin NLS sequence, and a T2A sequence.

In some embodiments, the nucleotide sequence encoding the tracrRNA comprises SEQ ID NO: 12966. In some embodiments, the nucleotide sequence encoding a tracrRNA containing an iBAR sequence for barcode sequence analysis comprises SEQ ID NO: 12967, wherein positions labeled as “n” comprise a distinct 6 nucleotide length barcode sequence per each sgRNA comprising a specific gRNA target. In some embodiments of methods described herein, iBAR sequences corresponding to particular sgRNA constructs are sequenced from genomic DNA extracted following CRISPRi targeting in cells from a cell sample to determine the relative frequency of particular sgRNA constructs present in a library of sgRNA constructs compared to other representatives of the library. In some embodiments, the plasmid vector comprises a random sequence label (RSL) within an i7 index binding site. In some embodiments of methods described herein, RSL sequences corresponding to particular sgRNA constructs of a vector are sequenced from genomic DNA extracted following CRISPRi targeting in cells from a cell sample to determine the relative frequency of particular sgRNA constructs present in a library of sgRNA constructs compared to other representatives of the library. In some embodiments, the nucleotide sequence for the i7 index binding site and RSL comprise SEQ ID NO: 12979 wherein “n” represents a specific RSL nucleotide sequence for each vector containing a distinct sgRNA. In some embodiments, the nucleotide sequence encoding the tracrRNA comprises SEQ ID NO: 12968. In some embodiments, the nucleotide sequence encoding the tracrRNA comprises SEQ ID NO: 12969. In some embodiments, the gRNA sequence and the tracrRNA sequence are configured to form an sgRNA expressed from an upstream human U6 promoter sequence. Due the specific gRNA sequence incorporated into the sgRNA, the sgRNA will target a particular location in the human genome for transcriptional repression of a target gene. In some embodiments, the gRNA sequence is selected from one of SEQ ID NOs: 55-459. In some embodiments, the gRNA sequence is selected from one of SEQ ID NOs: 460-1669. In some embodiments, the gRNA sequence is selected from one of SEQ ID NOs: 1670-8119. In some embodiments, the gRNA sequence is selected from one of SEQ ID NOs: 8120-12949. In some embodiments, PAM sequence of NGG is found directly downstream of the gRNA target sequence in the non-target strand of genomic DNA of cells from the cell sample.

In some embodiments, the epigenetic modulator sequence is a sequence encoding a KRAB domain. In some embodiments, the epigenetic modulator sequence is a sequence encoding a ZNF10 KRAB domain. In some embodiments, the epigenetic modulator sequence is a sequence encoding a ZFP1 KRAB domain. In some embodiments, the epigenetic modulator sequence is a sequence encoding a ZFP14 KRAB domain. In some embodiments, the epigenetic modulator sequence is a sequence encoding a ZFP82 KRAB domain. In some embodiments, the epigenetic modulator sequence is a sequence encoding a ZIM2 KRAB domain. In some embodiments, the epigenetic modulator sequence is a sequence encoding a ZIM3 KRAB domain. In some embodiments, the epigenetic modulator sequence is a sequence encoding a ZNF140 KRAB domain. In some embodiments, the epigenetic modulator sequence is a sequence encoding a ZNF175 KRAB domain. In some embodiments, the epigenetic modulator sequence is a sequence encoding a ZNF18 KRAB domain. In some embodiments, the epigenetic modulator sequence is a sequence encoding a ZNF250 KRAB domain. In some embodiments, the epigenetic modulator sequence is a sequence encoding a ZNF37A KRAB domain. In some embodiments, the epigenetic modulator sequence is a sequence encoding a ZNF419 KRAB domain. In some embodiments, the epigenetic modulator sequence is a sequence encoding a ZNF595 KRAB domain. In some embodiments, the epigenetic modulator sequence is a sequence encoding a ZNF60 KRAB domain. In some embodiments, the epigenetic modulator sequence is a sequence encoding a ZNF8 KRAB domain. In some embodiments, the Cas enzyme sequences is a dCas9 sequence. In some embodiments, an SV40 poly adenylation sequence is positioned downstream of the 3′ LTR. In some embodiments, a sequence coding from an antibiotic resistance gene is position downstream of the SV40 poly adenylation sequence and oriented in the opposite orientation. In some embodiments, the sequence coding from an antibiotic resistance gene is an AmpR sequence encoding for a molecule providing resistance to ampicillin. In some embodiments, an AmpR promoter sequence is positioned upstream of the AmpR sequence. In some embodiments, an origin of replication (Ori) sequence is positioned downstream of the sequence coding from an antibiotic resistance gene in the same orientation.

### Epigenetic Modulators for Use in CRISPRi

A variety of epigenetic modulator protein domains are known to exist. One category of epigenetic modulator protein domain is a transcriptional repressor domain. Active transcriptional repressor domains are generally believed to produce repression of DNA transcription at a particular genomic locus by actively inhibiting the activation of transcription through interactions with other cellular proteins, such as basal transcription factors, transcriptional activator proteins or transcriptional coactivator proteins, and corepressors at a given genomic locus. Transcriptional repressors can further recruit other corepressors to a genomic location to further inhibit transcription. As such, the activity of transcriptional repressors and transcriptional repressor domains is dependent on both their ability to interact with other transcriptional regulators and on their ability to be directed or recruited within a close proximity to genomic locus to have its transcription repressed.

In some embodiments described herein, an epigenetic modulator that is capable of repressing transcription is produced as a fusion protein further comprising a Cas enzyme. In some embodiments, the Cas enzyme is a mutated Cas enzyme or a dCas. In some embodiments, the epigenetic modulator is a transcriptional repressor. In some embodiments, the transcriptional repressor is a full length protein. In some embodiments, the transcriptional repressor is a protein domain having transcriptional repressor functional properties. In some embodiments, the transcriptional repressor comprises a Krüppel associated box (KRAB) domain. A KRAB domain is a category of transcriptional repressor domain present in numerous zinc finger transcription factors. In some embodiments, the KRAB domain is between 40-200 amino acids in length. In some embodiments, the KRAB domain is between 45-100 amino acids in length. In some embodiments, a typical KRAB domain contains about 75 amino acid residues. In some embodiments, the minimal repression module for a KRAB domain contains about 45 amino acid residues. In some embodiments, the KRAB domain used to repress transcription at a genomic locus of interest is a ZNF10 KRAB domain. In some embodiments, the KRAB domain used to repress transcription at a genomic locus of interest is a ZFP1 KRAB domain. In some embodiments, the KRAB domain used to repress transcription at a genomic locus of interest is a ZFP14 KRAB domain. In some embodiments, the KRAB domain used to repress transcription at a genomic locus of interest is a ZFP84 KRAB domain. In some embodiments, the KRAB domain used to repress transcription at a genomic locus of interest is a ZIM2 KRAB domain. In some embodiments, the KRAB domain used to repress transcription at a genomic locus of interest is a ZIM3 KRAB domain. In some embodiments, the KRAB domain used to repress transcription at a genomic locus of interest is a ZNF140 KRAB domain. In some embodiments, the KRAB domain used to repress transcription at a genomic locus of interest is a ZNF175 KRAB domain. In some embodiments, the KRAB domain used to repress transcription at a genomic locus of interest is a ZNF18 KRAB domain. In some embodiments, the KRAB domain used to repress transcription at a genomic locus of interest is a ZNF250 KRAB domain. In some embodiments, the KRAB domain used to repress transcription at a genomic locus of interest is a ZNF37A KRAB domain. In some embodiments, the KRAB domain used to repress transcription at a genomic locus of interest is a ZNF419 KRAB domain. In some embodiments, the KRAB domain used to repress transcription at a genomic locus of interest is a ZNF595 KRAB domain. In some embodiments, the KRAB domain used to repress transcription at a genomic locus of interest is a ZNF60 KRAB domain. In some embodiments, the KRAB domain used to repress transcription at a genomic locus of interest is a ZNF8 KRAB domain. In some embodiments, epigenetic modulator used to repress transcription at a genomic locus of interest is selected from TABLE 1 or TABLE 2.

### CRISPRi System

In an aspect described herein is a CRISPR interference (CRISPRi) system comprising a single vector transfer plasmid. In some embodiments, the CRISPRi system comprises a composition for use in targeted knockdown of expression of one or more target genes. In some embodiments, the CRISPRi system comprises a composition for use in targeted repression of mRNA expression of one or more target genes. In some embodiments, the CRISPRi system is formulated into a solution for use in targeted knockdown of expression of one or more target genes. In some embodiments, the CRISPRi system is formulated into a solution for use in targeted knockdown of expression of one target gene. In some embodiments, the one target gene is listed in Table 9A. In some embodiments, the one target gene is targeted with a gRNA sequence produced from a gene sequence listed in SEQ ID NOs: 44-459. In some embodiments, the one target gene is listed in Table 9B. In some embodiments, the one target gene is targeted with a gRNA sequence produced from a gene sequence listed in SEQ ID NOs: 460-1669. In some embodiments, the one target gene is a kinase target gene listed in Table 9C. In some embodiments, the one target gene is targeted with a gRNA sequence produced from a gene sequence listed in SEQ ID NOs: 1670-8119. In some embodiments, the one target gene is an IgSF target gene listed in Table 9D. In some embodiments, the one target gene is targeted with a gRNA sequence produced from a gene sequence listed in SEQ ID NOs: 8120-12949. In some embodiments, the CRISPRi system comprises 1, 2, 3, 4, 5, 6 or more separate sgRNA sequences that target a selected target gene to knockdown mRNA expression of the selected target gene. In some embodiments, the CRISPRi system is formulated into a solution for use in targeted knockdown of expression of two or more target genes. In some embodiments, the CRISPRi system is formulated into a solution for use in targeted knockdown of expression of a plurality of genes listed in Table 9A. In some embodiments, the CRISPRi system is formulated into a solution for use in targeted knockdown of expression of a plurality of genes listed in Table 9B. In some embodiments, the CRISPRi system is formulated into a solution for use in targeted knockdown of expression of a plurality of genes listed in Table 9C. In some embodiments, the CRISPRi system is formulated into a solution for use in targeted knockdown of expression of a plurality of genes listed in Table 9D. In some embodiments, the CRISPRi system is formulated into a single-tube solution for use in targeted knockdown of expression one or more target genes. In some embodiments, the CRISPRi system is formulated into a single-tube solution for use in targeted knockdown of expression one target genes. In some embodiments, the CRISPRi system is formulated into a single-tube solution for use in targeted knockdown of expression a plurality of target genes. In some embodiments, a single gene is targeted by the CRISPRi system. In some embodiments, a single gene is targeted by the CRISPRi system by one gRNA sequence. In some embodiments, the gRNA is formatted as an sgRNA. In some embodiments, a single gene is targeted by the CRISPRi system by two or more distinct gRNA sequences targeting the same target gene. In some embodiments, a single gene is targeted by the CRISPRi system by three or more distinct gRNA sequences targeting the same target gene. In some embodiments, a single gene is targeted by the CRISPRi system by four or more distinct gRNA sequences targeting the same target gene. In some embodiments, a single gene is targeted by the CRISPRi system by five, six, seven, eight, nine, ten or more distinct gRNA sequences targeting the same target gene. In some embodiments, the gRNA sequences targeting the same gene overlap. In some embodiments, the gRNA sequences targeting the same gene comprise non-overlapping gRNA sequences. In some embodiments, a pool of two or more genetically distinct lentiviral particles, each genetically distinct lentiviral particle comprise a single vector encoding a non-overlapping gRNA and a CAS endonuclease, wherein each non-overlapping gRNA targets a non-overlapping sense or an antisense strand of a gene sequence. In some embodiments, a pool of gRNA sequences target two or more target genes. In some embodiments, a pool of gRNA sequences target three or more target genes. In some embodiments, a pool of gRNA sequences target four or more target genes. In some embodiments, a pool of gRNA sequences target five or more target genes. In some embodiments, a pool of gRNA sequences target six, seven, eight, nine, ten or more target genes. In some embodiments, a pool of gRNA sequences target a plurality of target genes listed in Table 9A. In some embodiments, a pool of gRNA sequences target a plurality of target genes listed in Table 9B. In some embodiments, a pool of gRNA sequences target a plurality of target genes listed in Table 9C. In some embodiments, a pool of gRNA sequences target a plurality of target genes listed in Table 9D. In some embodiments, the CRISPRi system comprising a single vector transfer plasmid system comprises: a) a first promoter operably linked to a first nucleotide sequence encoding a catalytically inactive RNA-directed nuclease fused to a transcriptional repressor, and b) a second promoter operably linked to a second nucleotide sequence encoding a single-guide RNA (sgRNA) sequence comprising a CRISPR RNA (crRNA) sequence having complementarity to at least a portion of a gene that encodes a protein target and a trans-activating CRISPR RNA (tracrRNA) sequence. In some embodiments the crRNA sequence comprises at least about 80%, 85%, 87%, 90%, 92%, 95%, or 100% sequence identity to a gRNA sequence selected from SEQ ID NOs: 55-12949. In some embodiments the crRNA sequence is identical to a gRNA sequence selected from SEQ ID NOs: 55-12949.

In some embodiments, the CRISPRi system comprises a single vector transfer plasmid system comprising: a) a first promoter operably linked to a first nucleotide sequence encoding a catalytically inactive RNA-directed nuclease fused to a transcriptional repressor; and b) a second promoter operably linked to a second nucleotide sequence encoding a single-guide RNA (sgRNA) sequence comprising a CRISPR RNA (crRNA) sequence having complementarity to at least a portion of a gene that encodes a protein target and a trans-activating CRISPR RNA (tracrRNA) sequence. In some embodiments, the second nucleotide sequence encodes an sgRNA sequence comprising a nucleic acid sequence corresponding to at least 5 contiguous bases and up to at most 30 contiguous bases from the sense strand or the antisense strand of a gene sequence selected from SEQ ID NOs: 55-12949. In some embodiments, the second nucleotide sequence encodes an sgRNA sequence comprising a nucleic acid sequence corresponding to the sense strand or the antisense strand of a gene sequence selected from SEQ ID NOs: 55-12949. In some embodiments, the second nucleotide sequence encodes an sgRNA sequence comprising a nucleic acid sequence corresponding to the sense strand or the antisense strand of a gene sequence selected from SEQ ID NOs: 55-459. In some embodiments, the second nucleotide sequence encodes an sgRNA sequence comprising a nucleic acid sequence corresponding to the sense strand or the antisense strand of a gene sequence selected from SEQ ID NOs: 460-1669. In some embodiments, wherein the second nucleotide sequence encodes an sgRNA sequence comprising a nucleic acid sequence corresponding to the sense strand or the antisense strand of a gene sequence selected from SEQ ID NOs: 1670-8119. In some embodiments, the second nucleotide sequence encodes an sgRNA sequence comprising a nucleic acid sequence corresponding to the sense strand or the antisense strand of a gene sequence selected from SEQ ID NOs: 8120-12949. In some embodiments, the sgRNA comprise a tracrRNA sequence encoded by the nucleotide sequence of SEQ ID NO: 12966. In some embodiments, the sgRNA comprise a tracrRNA sequence encoded by the nucleotide sequence of SEQ ID NO: 12967. In some embodiments, the sgRNA comprise a tracrRNA sequence encoded by the nucleotide sequence of SEQ ID NO: 12968. In some embodiments, the sgRNA comprise a tracrRNA sequence encoded by the nucleotide sequence of SEQ ID NO: 12969. Exemplary nucleotide sequences for use in constructing the single vector transfer plasmid are listed in TABLE 14. In some embodiments, a U6 promoter sequence is used to drive expression of an sgRNA. In some embodiments, the U6 promoter sequences comprises SEQ ID NO: 12964. In some embodiments, the U6 promoter sequences comprises SEQ ID NO: 12963. In some embodiments, a minimal compound pol-III promoter M11 sequence is used to drive expression of an sgRNA. In some embodiments, the minimal compound pol-III promoter M11 sequence comprises SEQ ID NO: 12965. In some embodiments, a UbiC promoter sequence is used to drive expression of an mRNA encoding a dCas fused to a transcriptional repressor domain. In some embodiments, the UbiC promoter sequence comprises SEQ ID NO: 12970. In some embodiments, the CRISPRi system comprises a nucleotide sequence encoding a T2A self-cleaving peptide. In some embodiments, the CRISPRi system comprises a nucleotide sequence encoding a marker gene. In some embodiments, the marker gene encodes a fluorescent protein. In some embodiments, the fluorescent protein comprises GFP, eGFP, T-Shappire, EBFP2, m Turquoise, mTFP1, Topaz, YPet, ZsYellow1, mOrange, DsREd, mRuby, mCherry, sfGFP, or muGFP. In some embodiments, the marker gene comprises Thy 1. In some embodiments, the CRISPRi system comprises a 5′ LTR sequence and a 3′ LTR sequence. In some embodiments, the CRISPRi system comprises an iBar barcode. In some embodiments, the CRISPRi system comprises a random sequence label (RSL) within an i7 index binding site.

In some embodiments, the single vector transfer plasmid system comprises a single vector transfer plasmid formatted for co-transfection with one or more viral packaging plasmids. In some embodiments, the CRISPRi system comprises one or more viral packaging plasmids. In some embodiments, the one or more viral packaging plasmids comprise nucleotide coding sequence to encode lentiviral particles, retroviral particles, adenoviral particles, adeno-associated viral particles, or herpes simplex viral particles. In some embodiments, the one or more viral packaging plasmids comprise nucleotide coding sequence to encode lentiviral particles. In some embodiments, the one or more viral packaging plasmids comprise nucleotide coding sequence to encode retroviral particles. In some embodiments, the one or more viral packaging plasmids comprise nucleotide coding sequence to encode adenoviral particles. In some embodiments, the one or more viral packaging plasmids comprise nucleotide coding sequence to encode adeno-associated viral particles. In some embodiments, the one or more viral packaging plasmids comprise nucleotide coding sequence to encode herpes simplex viral particles.

In some embodiments, the CRISPRi system comprises a single vector transfer plasmid system comprising: a) a first promoter operably linked to a first nucleotide sequence encoding a catalytically inactive RNA-directed nuclease fused to a transcriptional repressor; and b) a second promoter operably linked to a second nucleotide sequence encoding a single-guide RNA (sgRNA) sequence comprising a CRISPR RNA (crRNA) sequence having complementarity to at least a portion of a gene that encodes a protein target and a trans-activating CRISPR RNA (tracrRNA) sequence, wherein a) and b) are contained within the single vector transfer plasmid. In some embodiments, the single vector transfer plasmid has a plasmid size of less than about 9.2 kilobases (kb), 9 kb, 8.6 kb, 8.5 kb, 8 kb, 7.5 kb, 7 kb, 6.5 kb, 6 kb, 5.5 kb, 5 kb, 4.5 kb, 4 kb, 3.5 kb, 3 kb, 2.5 kb, or 2 kb. In some embodiments, the single vector transfer plasmid has a plasmid size of at least about 8.6 kilobases (kb), 8.5 kb, 8 kb, 7.5 kb, 7 kb, 6.5 kb, 6 kb, 5.5 kb, 5 kb, 4.5 kb, 4 kb, 3.5 kb, 3 kb, 2.5 kb, or 2 kb. In some embodiments, the single vector transfer plasmid has a plasmid size of about 8.7 kb.

In some embodiments, the CRISPRi system comprises a single vector transfer plasmid system comprising: a) a first promoter operably linked to a first nucleotide sequence encoding a catalytically inactive RNA-directed nuclease fused to a transcriptional repressor; and b) b) a second promoter operably linked to a second nucleotide sequence encoding a single-guide RNA (sgRNA) sequence comprising a CRISPR RNA (crRNA) sequence having complementarity to at least a portion of a gene that encodes a protein target and a trans-activating CRISPR RNA (tracrRNA) sequence, wherein the catalytically inactive RNA-directed nuclease is dCas9, dCas12a, dCas12f1, dCasMINI, or a variant thereof. In some embodiments, the catalytically inactive RNA-directed nuclease is dCas9 or a variant thereof. In some embodiments, the catalytically inactive RNA-directed nuclease is dCas12a or a variant thereof. In some embodiments, the catalytically inactive RNA-directed nuclease is dCas12f1 or a variant thereof. In some embodiments, the catalytically inactive RNA-directed nuclease is dCasMINI or a variant thereof. In some embodiments, the first nucleotide sequence comprises coding sequence for a dCas9 polypeptide, a dCas12 polypeptide, a dCas12f1 polypeptide, or a dCasMINI polypeptide, or a variant thereof. In some embodiments, the coding sequence for the dCas9 polypeptide encodes a dCas9 polypeptide at least about 90% identical to the amino acid sequence of SEQ ID NO: 12950. In some embodiments, the coding sequence for the dCas9 polypeptide encodes a dCas9 polypeptide comprising the amino acid sequence of SEQ ID NO: 12950. In some embodiments, the coding sequence for the dCas12a polypeptide encodes a dCas 12a polypeptide at least about 90% identical to the amino acid sequence of SEQ ID NO: 12951. In some embodiments, the coding sequence for the dCas12a polypeptide encodes a dCas12a polypeptide comprising the amino acid sequence of SEQ ID NO: 12951. In some embodiments, the coding sequence for the dCas 12f1 polypeptide encodes a dCas12f1 polypeptide at least about 90% identical to the amino acid sequence of SEQ ID NO: 12952. In some embodiments, the coding sequence for the dCas 12f1 polypeptide encodes a dCas12f1 polypeptide comprising the amino acid sequence of SEQ ID NO: 12952. In some embodiments, the coding sequence for the dCasMINI polypeptide encodes a dCasMINI polypeptide at least about 90% identical to the amino acid sequence of SEQ ID NO: 12953. In some embodiments, the coding sequence for the dCasMINI polypeptide encodes a dCasMINI polypeptide comprising the amino acid sequence of SEQ ID NO: 12953.

In some embodiments, the CRISPRi system comprises a first promoter operably linked to a first nucleotide sequence encoding a catalytically inactive RNA-directed nuclease fused to a transcriptional repressor. In some embodiments, the transcriptional repressor comprises a sequence of KRAB (also referred to as KOX), SID, MBD2, MBD3, HP1a, DNMT family (including DNMT1, DNMT3A, DNMT3B, DNMT3L, DNMT2A), Sin3a, Rb, MeCP2, ROM2, AtHD2A, LSD1, SUV39H1, G9a (EHMT2), ZFP1, ZFP14, ZFP82, ZIM2, ZIM3, ZNF18, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF419, ZNF140, ZNF175, ZNF214, ZNF184, ZNF8, ZNF60, ZNF595, ZNF596, ZNF10, ZNF37A, ZNF394, ZNF610, ZNF273, ZNF34, ZNF250, ZNF98, ZNF675, ZNF213, ZFP28-2, ZNF224, ZNF257, GLP (EHMT2), or a variant or combination thereof. In some embodiments, the transcriptional repressor comprises a sequence of KRAB. In some embodiments, the KRAB comprises a KRAB protein domain, or variant thereof, derived from one or more of genes listed in Table 1 or Table 2. In some embodiments, the KRAB protein domain, or variant thereof, comprises a sequence at least about 90%, 91%, 92%, 93%, 94%, 95%, 96%, 97%, or 98% identical to a portion of at least 60 contiguous amino acids of a sequence selected from SEQ ID NOs: 1-54. In some embodiments, the transcriptional repressor comprises a KRAB protein domain derived from ZFP1, ZFP14, ZFP82, ZIM2, ZIM3, ZNF140, ZNF175, ZNF18, ZNF250, ZNF37A, ZNF419, ZNF595, ZNF60, or ZNF8. In some embodiments, the transcriptional repressor comprises a KRAB protein domain comprising an amino acid sequence corresponding to the KRAB domain coordinates listed in Table 1 or Table 2.

In some embodiments of the CRISPRi system, the catalytically inactive RNA-directed nuclease is fused to the transcriptional repressor via a peptide linker. In some embodiments, the catalytically inactive RNA-directed nuclease fused to the transcriptional repressor is contained within a single polypeptide chain and the catalytically inactive RNA-directed nuclease is separated from the transcriptional repressor by a peptide linker comprising about 1 to about 100 amino acids. In some embodiments, the peptide linker comprises a flexible linker. In some embodiments, the flexible linker is about 10, 11, 12, 13, 14, 15, 16, 17, 18, 19, 20, 21, 22, 23, 24, 25, 26, 27, 28, 29, or 30 amino acids in length. In some embodiments, the peptide linker comprises SEQ ID NO: 12954. In some embodiments, the peptide linker consists essentially of the amino acid sequence of SEQ ID NO: 12954.

In some embodiments of the CRISPRi system, the catalytically inactive RNA-directed nuclease comprises a nuclear localization sequence (NLS) positioned amino-terminal (N-terminal) to a RuvC-1 domain of the CAS endonuclease. In some embodiments, the NLS positioned N-terminal to the RuvC-1 domain comprises a triple SV40 NLS. In some embodiments of the CRISPRi system, the catalytically inactive RNA-directed nuclease comprises an NLS positioned carboxy-terminal (C-terminal) to a RuvC-III domain of the CAS endonuclease. In some embodiments, the NLS positioned C-terminal to the RuvC-III domain comprises a single nucleoplasmin NLS. In some embodiments of the CRISPRi system, the catalytically inactive RNA-directed nuclease comprises a peptide-linked downstream reporter C-terminal to a RuvC-III domain of the catalytically inactive RNA-directed nuclease. In some embodiments, the peptide-linked downstream reporter C-terminal to the RuvC-III domain comprises a self-cleaving T2A-peptide linked downstream reporter. In some embodiments, the self-cleaving T2A-peptide linked downstream reporter is linked to the catalytically inactive RNA-directed nuclease C-terminal relative to the NLS positioned C-terminal to the RuvC-III domain. In some embodiments, the self-cleaving T2A-peptide linked downstream reporter comprises Thy 1. In some embodiments of the CRISPRi system, the single vector encodes a first promoter sequence, wherein the first promoter sequence is operably-linked to coding sequence of the catalytically inactive RNA-directed nuclease. In some embodiments, the first promoter sequence comprises a UbiC promoter sequence. In some embodiments of the CRISPRi system, the single vector encodes a second promoter sequence, wherein the second promoter sequence is operably-linked to coding sequence of the sgRNA. In some embodiments, the second promoter sequence comprises a U6 promoter sequence. In some embodiments of the CRISPRi system, the single vector encodes a 5′ LTR sequence and a 3′ LTR sequence. In some embodiments, the 5′ LTR sequence and/or the 3′ LTR sequence comprises a deletion that creates a self-inactivating vector. In some embodiments, the nucleotide sequence encoding the 5′ LTR comprises SEQ ID NO: 12962. In some embodiments, the nucleotide sequence encoding the full length Human U6 promoter comprises SEQ ID NO: 12963. In some embodiments, the nucleotide sequence encoding the minimal human U6 promoter comprises SEQ ID NO: 12964. In some embodiments, the nucleotide sequence encoding the T2A comprises SEQ ID NO: 12971. In some embodiments, the nucleotide sequence encoding the T2A with linker elements comprises SEQ ID NO: 12972. In some embodiments, the nucleotide sequence encoding the Thy 1 marker sequence comprises SEQ ID NO: 12973. In some embodiments, the nucleotide sequence encoding the 3′ LTR sequence comprises SEQ ID NO: 12974. In some embodiments, the nucleotide sequence encoding the 3×SV40 NLS comprises SEQ ID NO: 12981.

In some aspects, the CRISPRi system described herein is formulated into a composition. In some embodiments, the composition is contained within a single tube. In some embodiments, the composition comprises a high titer CRISPRi lentiviral gene modulation solution comprising the CRISPRi system described herein. In some embodiments, the high titer CRISPRi lentiviral gene modulation solution is sufficient to transduce a majority of primary cells maintained in culture, wherein the primary cells are derived from a subject. In some embodiments, the high titer CRISPRi lentiviral gene modulation solution is sufficient to transduce at least about 50%, 55%, 60%, 65%, 70%, 75%, 80%, 85%, 90%, 95%, 98%, or 99% of primary cells maintained in culture, wherein the primary cells are derived from a subject. In some embodiments, the high titer CRISPRi lentiviral gene modulation solution is concentrated to a titer of at least 1×10⁷, 2×10⁷, 3×10⁷, 4×10⁷, 5×10⁷, 6×10⁷, 7×10⁷, 8×10⁷, 9×10⁷, or 1×10⁸ Infectious Units/mL. In some embodiments, the high titer CRISPRi lentiviral gene modulation solution is concentrated to a titer of at most about 1×10⁸ or 5×10⁷ Infectious Units/mL. In some embodiments, the high titer CRISPRi lentiviral gene modulation solution is concentrated to a titer between about 1×10⁷-1×10⁸ Infectious Units/mL.

### RNAi and Other Non-CRISPRi Gene Repression

In one aspect, provided herein is a library comprising a plurality of gene modulatory reagents, wherein one or more of the modulatory reagents comprise a short hairpin RNA (shRNA) complementary to a target mRNA of a protein targeted by a known therapeutic agent (e.g., a therapeutic agent chosen from TABLES 3, 4, 10A, 10B, and 10C). Non-limiting examples of target proteins include those encoded by the genes listed in TABLE 9A, TABLE 9B, TABLE 9C, and TABLE 9D. In some embodiments, one or more of the shRNA each comprise a sequence at least about 85%, 90%, 95%, or 100% complementary to at least about 10, 15, or 20 contiguous nucleobases of a target mRNA. In some embodiments, one or more of the shRNA each comprise a sequence at least about 85%, 90%, 95%, or 100% complementary to at least about 10, 15, or 20 contiguous nucleobases of a target mRNA encoding for a protein selected from TABLE 9A, TABLE 9B, TABLE 9C, and TABLE 9D. The library may comprise from about 10 to about 2,000, from about 50 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different shRNA sequences.

In some instances, the knocking down of the function of the target comprises use of an oligonucleotide to repress expression of the target. In some embodiments, the oligonucleotide comprises antisense oligonucleotides (ASOs), short interfering RNAs (siRNAs), aptamers and microRNA (miRNA) inhibitors (anti-miRs) and mimics (pro-miRs).

In some instances, the knocking down of the function of the target comprises use of a transcription activator-like effector nucleases (TALEN) to repress expression of the target.

In some instances, the knocking down of the function of the target comprises use of zinc finger proteins and a repressor domain to repress expression of the target bound by the zinc finger proteins.

### Targeted Pharmacologic Landscape

In one aspect, provided herein is a pharmacologic landscape comprising a compilation of therapeutic agents having known protein targets, referred to as a drug compilation. The drug compilation may include low molecular weight drugs (e.g., having a molecule weight less than about 1 kDa) and biologic drugs (e.g., proteins such as antibodies). The drug compilation may comprise drugs suitable for a patient's particular disease or condition, e.g., hematological malignant cancers. In various embodiments, the drug compilation includes, but is not limited to, FDA-approved therapeutic agents and as such may be expanded as new drugs are developed. The drug compilation may include, but is not limited to, all or nearly all of the targeted drugs treating a particular class of disease, e.g., the drug compilation includes at least about 50%, at least about 60%, at least about 70%, at least about 80%, at least about 90%, or 100% of known FDA-approved drugs for a particular disease class having a known protein target. Also provided herein are compilations of a particular target classes of interest (e.g., G-protein coupled receptors, kinases, etc.). In some embodiments, a drug compilation comprises drugs listed in TABLE 3, TABLE 4, TABLE 10A, TABLE 10B, or TABLE 10C, or any combination thereof.

### Gene Target Libraries

Further provided herein is a library of genetic targets comprising the genes encoding the proteins targeted by the therapeutic agents in the drug library. For therapeutic agents that are non-specific inhibitors, such as multi-kinase inhibitors, the targets may include, but are not limited to, multiple gene targets. The number of targeted genes must be significantly smaller than the “whole genome,” generating a compact library amenable to both in vitro and in vivo analysis. Non-limiting examples of targeted genes are shown TABLE 9A, TABLE 9B, TABLE 9C, and TABLE 9D.

Non-limiting examples of targeted genes for oncology are shown in TABLES 9A-9B. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 genes from TABLE 9A. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 genes from TABLE 9B. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 genes from TABLE 9C. The targeted genes described herein may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 genes from TABLE 9D.

### Compilation of Modified Cancer Cells

Further provided herein are compilations of modified cancer cells. An exemplary compilation comprises a plurality of modified cancer cells, wherein each modified cancer cell harbors one or more gene modulatory reagents, and each gene modulatory reagent is capable of knocking down or knocking out the function of a gene that encodes a protein target from a library of protein targets. In some embodiments, the library of protein targets comprises at least 1, at least 2, at least 3, at least 4, at least 5, at least 10, or at least 20 protein targets of TABLE 9A. In some embodiments, the library of protein targets comprises at least 1, at least 2, at least 3, at least 4, at least 5, at least 10, or at least 20 protein targets of TABLE 9B. In some embodiments, the library of protein targets comprises at least 1, at least 2, at least 3, at least 4, at least 5, at least 10, or at least 20 protein targets of TABLE 9C. In some embodiments, the library of protein targets comprises at least 1, at least 2, at least 3, at least 4, at least 5, at least 10, or at least 20 protein targets of TABLE 9D. In some embodiments, the modified cancer cells are modified primary cancer cells. The modified cancer cells may comprise from about 10 to about 2,000, from about 50 to about 2,000, from about 10 to about 500, from about 10 to about 200, from about 10 to about 150, from about 50 to about 500, from about 50 to about 200, from about 100 to about 2,000, or from about 500 to about 2,000 different populations of modified cancer cells.

The modified cancer cells may comprise from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents. At least one of the one or more gene modulatory reagents may comprise a sequence selected from SEQ ID NOS: 55-459, 460-1669. At least one of the one or more of gene modulatory reagents may comprise a sequence at least about 90% identical to a sequence selected from SEQ ID NOS: 55-1669. At least one of the one or more gene modulatory reagents may comprise a sequence selected from SEQ ID NOS: 55-459. At least one of the one or more of gene modulatory reagents may comprise a sequence at least about 90% identical to a sequence selected from SEQ ID NOS: 55-459. At least one of the one or more gene modulatory reagents may comprise a sequence selected from SEQ ID NOS: 460-1669. At least one of the one or more of gene modulatory reagents may comprise a sequence at least about 90% identical to a sequence selected from SEQ ID NOS: 460-1669. At least one of the one or more gene modulatory reagents may comprise a sequence selected from SEQ ID NOS: 1670-8119. At least one of the one or more of gene modulatory reagents may comprise a sequence at least about 90% identical to a sequence selected from SEQ ID NOS: 1670-8119. At least one of the one or more gene modulatory reagents may comprise a sequence selected from SEQ ID NOS: 8120-12949. At least one of the one or more of gene modulatory reagents may comprise a sequence at least about 90% identical to a sequence selected from SEQ ID NOS: 8120-12949.

The modified cancer cells may have been modified by CRISPR-based methods. As such, the gene modulatory reagents harbored by the modified cancer cells may comprise a gRNA sequence comprising homology to at least a portion of the gene whose function is knocked down or knocked out in the modified cancer cell. In some cases, the gRNA comprises homology to about 10 to about 50 contiguous nucleotides of the gene. The homology may be at least about 90% sequence identity. The gRNA may be positioned within a vector, e.g., for viral delivery as discussed herein. The gRNA sequence may be formatted as an sgRNA sequence comprising a gRNA and tracrRNA sequence. In some embodiments, DNA encoding the sgRNA sequence is integrated into the genome of cells of the cell sample to be expressed from a vector sequence delivered to cells of the cell sample by a lentiviral vector.

The modified cancer cells may also comprise an endonuclease, for instance, where the cells are modified using CRISPR-based methods comprising CRISPRn, or CRISPRi. The endonuclease may comprise a Cas9 or Cas12a endonuclease. Non-limiting examples of Cas9 or Cas12a endonuclease include, but are not limited to, S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpf1), Lachnospiraceae bacterium (LbCpf1), AsCpf1 RR variant, LbCpf1 RR variant, AsCpf1 RVR variant, C. jejuni Cas9 (CjCas9), N. meningitidis (NmCas9), S. thermophilus (StCas9), T. denticola (TdCas9), and Mad7. The endonuclease may not comprise a Cas9 or Cas12a endonuclease.

In some instances, the endonuclease is a mutated endonuclease. In some embodiments, the mutated endonuclease comprises one or more mutation in nucleolytic domains and cannot cleave target DNA sequence but can still bind to the target DNA sequence. In some embodiments, the mutated endonuclease is derived from wildtype endonucleases such as Cas9, Cas12, Cas12a (or Cpf1 or Mad7), Cas12b (or C2c1 or Cpf2), Cas12c (C2c3), Cas12d (or CasY), Cas12e (or CasX), Cas13, Cas13a (or C2c2), Cas13b (or C2c6), Cas13c (or C2c7), Cas13d (or Casrx), Cas14, Cas14a, Cas14b, Cas14c, Casl, CaslB, Cas2, Cas3, Cas4, Cas5, Cas5e (CasD), Cas6, Casoe, Casof, Cas7, Cas8a, Cas8a1, Cas8a2, Cas8b, Cas8c, Csnl, Csx12, Cas10, Cas10d, CasIO, CaslOd, CasF, CasG, CasH, Csy1, Csy2, Csy3, Csel (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, CsxIO, Csx16, CsaX, Csx3, Csx1, Csx15, Csf1, Csf2, Csf3, Csf4, or Cul966, or derivative thereof, variant thereof, fragment thereof, or any combination thereof. In some embodiments, the mutated endonuclease is derived from the wildtype Cas9 or Cas12a family, which may include, but is not limited to, S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpf1), Lachnospiraceae bacterium (LbCpf1), AsCpf1 RR variant, LbCpf1 RR variant, AsCpf1 RVR variant, C. jejuni Cas9 (CjCas9), N. meningitidis (NmCas9), S. thermophilus (StCas9), T. denticola (TdCas9), and Mad7. In some instances, the mutated endonuclease described herein for use is CRISPRi.

The modified cancer cells may have been modified by gene silencing using shRNA gene modulatory reagents. Therefore, one or more of the gene modulatory reagents may comprise an shRNA sequence comprising homology to at least a portion of the gene whose function is knocked down in the modified cancer cell. The shRNA may comprise homology to about 10 to about 50 contiguous nucleotides of the gene. The homology may be at least about 90% sequence homology or identity. The shRNA may be positioned within a vector, e.g., for viral delivery as discussed herein.

In some embodiments, the modified cancer cells comprise depleting a target protein level during in vitro propagation of the modified cancer cells by at least 10%, 20%, 30%, 40%, 50%, 60%, 70%, 75%, 80%, 85%, 90%, 95%, or 99% compared to a target protein level prior to cancer cell modification.

In some embodiments, in order to determine whether the subject is susceptible to treatment, functional assay is performed. In some embodiments, the functional assay is an in vitro assay. In some embodiments, the functional assay comprises isolating cancer cells from the subject. In some embodiments, functional assay comprises determining if proliferation and/or viability of cancer cells isolated from the subject is reduced and/or inhibited after (i) knocking down the function of the target of the targeted cancer therapeutic or (ii) knocking out the target of the targeted cancer therapeutic

### Delivery Vehicle

In some embodiments, the components of CRISPR-based method are delivered to the cancer cells isolated from the subject in a multiple delivery vector system. In some embodiments, the multiple delivery vector system comprises multiple independent constructs encoding different gRNA sequences. In some embodiments, the multiple delivery vector system comprises at least 1, at least 2, at least 3, at least 4, at least 5, at least 6, at least 7, at least 8, at least 9, at least 10, at least 100, at least 1000, at least 10000, or at least 100000 construct(s). In some embodiments, the multiple delivery vector system comprises at least 1, at least 2, at least 3, at least 4, at least 5, at least 6, at least 7, at least 8, at least 9, at least 10, at least 100, at least 1000, at least 10000, or at least 100000 gRNAs. In some embodiments, the multiple delivery vector system comprises less than about 15000, 10000, 5000, 2000, 1250, or 500 distinct gRNAs. In some embodiments, the multiple delivery vector system comprises between about 20-100, 100-500, 200-450, 400-1210, 1200-1620, 1000-4830, or 1200-6450 distinct gRNAs.

In some embodiments, the components of CRISPR-based method are delivered to T cells isolated from a subject in a single delivery vector system. In some embodiments, the components of CRISPR-based method are delivered to T cells isolated from the subject in a split delivery vector system.

In a non-limiting embodiment of a single delivery vector system, one lentiviral transfer vector is used for expression of both gRNA(s) and endonuclease protein. In some embodiments, the single delivery vector system comprises at least 1, at least 2, at least 3, at least 4, at least 5, at least 6, at least 7, at least 8, at least 9, at least 10, at least 100, at least 300, at least 500, at least 700, at least 900, at least 1000, at least 1250, at least 1500, at least 2000, at least 2500, at least 3000, at least 4000, at least 5000, at least 10000, or at least 50000 gRNA(s), and up to about 100000 gRNAs. In some embodiments, the single delivery vector system comprises at most 1, at most 2, at most 3, at most 4, at most 5, at most 6, at most 7, at most 8, at most 9, at most 10, at most 100, at most 300, at most 500, at most 700, at most 900, at most 1000, at most 1250, at most 1500, at most 2000, at most 2500, at most 3000, at most 4000, at most 5000, at most 10000, or at most 50000 gRNA(s), and at most up to about 100000 gRNAs. In some embodiments, one or more gRNA(s) is carried by one lentiviral vector. In some embodiments, a sequence encoding one gRNA is carried by one lentiviral vector. In some embodiments, sequences encoding two gRNAs are carried by one lentiviral vector. In some embodiments, one or more gRNA(s) can target the same gene. In some embodiments, one or more gRNA(s) can target different genes. In some instances, the endonuclease is a dCas. In some instances, the dCas is a dCas9.

In some embodiments, a repressor domain is fused to the endonuclease protein. In some embodiments, nucleotide sequence encoding the repressor domain is fused to the 5′ end of nucleotide sequence encoding the endonuclease. In some embodiments, nucleotide sequence encoding the repressor domain is fused to the 5′ end of nucleotide sequence encoding the endonuclease via at a linker (e.g., a linker having least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids, and optionally flexible). In some embodiments, the repressor domain is fused to the N-terminus of the endonuclease via a linker (e.g., at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids, and optionally flexible). In some embodiments, nucleotide sequence encoding the repressor domain is fused to the 5′ end of nucleotide sequence encoding the endonuclease via a 16 amino acid flexible linker. In some embodiments, the repressor domain is fused to the N-terminus of the endonuclease via 16 amino acid flexible linker. In some embodiments, the amino acid flexible linker sequence comprises SEQ ID NO: 996.

In some embodiments, the repressor domain comprises KRAB (also referred to as KOX), SID, MBD2, MBD3, HP1a, DNMT family (including DNMT1, DNMT3A, DNMT3B, DNMT3L, DNMT2A), Sin3a, Rb, MeCP2, ROM2, AtHD2A, LSD1, SUV39H1, G9a (EHMT2), ZFP1, ZFP14, ZFP82, ZIM2, ZIM3, ZNF18, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF419, ZNF140, ZNF175, ZNF214, ZNF184, ZNF8, ZNF60, ZNF595, ZNF596, ZNF10, ZNF37A, ZNF394, ZNF610, ZNF273, ZNF34, ZNF250, ZNF98, ZNF675, ZNF213, ZFP28-2, ZNF224, ZNF257, GLP (EHMT2), NLuc, VP64, Rta, P16, P65, p300, TET1 catalytic domain, TDG, Ldb1 self-association domain, SAM activator (VP64, p65, HSF1), VPR (VP64, p65, Rta), CD, or SunTag domain, or a variant or combination thereof. In some embodiments, the repressor domain comprises KRAB domain derived from ZNF10 comprising amino acids 13-85 corresponding to the KRAB domain sequence from Uniprot ID Entry P21506. In some embodiments, the repressor domain comprises KRAB domain derived from ZFP1 sequence listed in SEQ ID NO: 1. In some embodiments, the repressor domain comprises KRAB domain derived from ZFP14 sequence listed in SEQ ID NO: 2. In some embodiments, the repressor domain comprises KRAB domain derived from ZFP82 sequence listed in SEQ ID NO: 3. In some embodiments, the repressor domain comprises KRAB domain derived from ZIM2 sequence listed in SEQ ID NO: 4. In some embodiments, the repressor domain comprises KRAB domain derived from ZIM3 sequence listed in SEQ ID NO: 5. In some embodiments, the repressor domain comprises KRAB domain derived from ZNF140 sequence listed in SEQ ID NO: 6. In some embodiments, the repressor domain comprises KRAB domain derived from ZNF175 sequence listed in SEQ ID NO: 7. In some embodiments, the repressor domain comprises KRAB domain derived from ZNF18 sequence listed in SEQ ID NO: 8. In some embodiments, the repressor domain comprises KRAB domain derived from ZNF250 sequence listed in SEQ ID NO: 14. In some embodiments, the repressor domain comprises KRAB domain derived from ZNF37A sequence listed in SEQ ID NO: 27. In some embodiments, the repressor domain comprises KRAB domain derived from ZNF419 sequence listed in SEQ ID NO: 31. In some embodiments, the repressor domain comprises KRAB domain derived from ZNF595 sequence listed in SEQ ID NO: 42. In some embodiments, the repressor domain comprises KRAB domain derived from ZNF60 sequence listed in SEQ ID NO: 44. In some embodiments, the repressor domain comprises KRAB domain derived from ZNF8 sequence listed in SEQ ID NO: 52. In some embodiments, the repressor domain is selected from TABLE 1. In some embodiments, the repressor domain is selected from TABLE 2.

In some embodiments, the single delivery vector is a lentiviral vector. In some embodiments, lentivirus are used to deliver CRISPR-Cas9 and selected sgRNA components into target cells derived from the subject. In some embodiments, the lentiviral vector is modified for high titer virus production. In some embodiments, the lentiviral vector comprises internal transcription units. In some embodiments, the internal transcription units comprise CRISPR components. In some embodiments, the CRISPR components comprises CRISPRi components. In some embodiments, the CRISPR components comprises one or more gRNA(s) and endonuclease. In some instances, the endonuclease is a dCas. In some instances, the dCas is a dCas9. In some embodiments, the lentiviral vector comprises CMV promoter located downstream of 5′ long term repeat (5′ LTR) to drive gene expression. In some embodiments, the lentiviral vector comprises U6 promoter located downstream of 5′ long term repeat (5′ LTR) to drive gene expression. In some embodiments, the lentiviral vector comprises UbiC promoter located downstream of 5′ long term repeat (5′ LTR) to drive gene expression. In some embodiments, the lentiviral vector comprises cis-acting viral elements, e.g., 5′LTR, packaging signal, extended Gag-coding region, posttranscriptional regulatory element (PRE), or central polypurine tract-central termination sequence (cPPT)/CTS. In some embodiments, the cis-acting viral elements are arranged in one continuous block located upstream of the CRISPR components.

In some embodiments, the internal transcription units are arranged in the same orientation as viral genome. Arrangement of internal transcription units, e.g., gRNA and endonuclease, in same orientation as viral genome can avoid generation of dsRNA during virus production, which can trigger protein kinase R (PKR) activation and suppress translation in the packaging cells. In some embodiments, the internal transcription units comprise CRISPR components. In some embodiments, the CRISPR components comprises CRISPRi component. In some embodiments, the CRISPR component comprises one or more gRNA(s) and endonuclease. In some instances, the endonuclease is a dCas. In some instances, the dCas is a dCas9. In some embodiments, the internal transcription units are arranged in the different orientation as viral genome.

In some embodiments, the lentiviral vector is modified to reduce the overall size of viral insert, e.g., omitting protein-based reporter and WPRE element that is commonly used in viral expression vector. In some embodiments, the single vector does not comprise a protein-based reporter and/or WPRE element.

In some embodiments, the lentiviral vector comprises a pseudotyped lentiviral vector. In some embodiments, the pseudotyped lentiviral vector comprises a plurality of envelope glycoproteins derived from Morbillivirus, Sendai virus (SeV), Nipah virus (NiV), Newcastle disease virus (NDV), or Baboon endogenous virus (BaEV), or a combination thereof. In some embodiments, the Morbillivirus is a measles virus (MV).

In some embodiments, the pseudotyped lentiviral vector comprises a VSV-G envelope protein. In some embodiments, the VSV-G envelope protein is fused to ZZ-type zinc finger domain (ZZ domain), which mediates binding to antibody Fc-domains. The ZZ domain peptide is an IgG Fc-binding peptide derived from protein A. This enables targeting of viral particles to target cell surface proteins mediated by an appropriate antibody that simultaneously binds the viral particle (via Fc domain) and the antibody target (via CDR). In some embodiments, the VSV-G envelope protein is co-expressed with the ZZ domain peptide. In some embodiments, one or more envelope glycoproteins are attached to an antibody or antigen binding domain of an antibody, e.g., a single-chain antibody fragment, nanobody, or darpin.

In some embodiments, the VSV-G envelope protein is fused to biotin-binding domain. Fusion of the VSV-G envelope protein with biotin-binding domains can be used for biotinylated affinity reagents, e.g., antibodies. In some embodiments, the VSV-G envelope protein is co-expressed with biotin-binding domain.

Expressing multiple gRNAs, or multiplexing, is a technique used in CRISPR technology to express multiple gRNAs from one plasmid. This technique can increase the probability of successful gene targeting/modulation. Multiple gRNAs can be expressed using a polycistronic transcript/precursor, which is when the mRNA encodes multiple proteins and its expression is controlled by a single promoter and a single terminator. In some embodiments, the polycistronic transcript/precursor can be utilized to express at least 1, at least 2, at least 3, at least 4, at least 5, at least 6, or at least 7 gRNAs. In some instances, the polycistronic transcript/precursor can be utilized to express one or more, 2 or more, 3 or more, 4 or more, 5 or more, 6 or more, or 7 or more gRNAs. In some embodiments, one or more gRNAs are expressed from a single vector. In some embodiments, one or more gRNAs are expressed via a polycistronic transcript. In some embodiments, the one or more gRNAs are expressed under the same promoter. In some embodiments, the one or more gRNAs are expressed under different promoters. In some embodiments, the one or more gRNAs are designed to target a single gene. In some embodiments, the one or more gRNAs are designed to target different genes.

The dCas 12a is smaller size compared to dCas9. The dCas 12a retains RNA processing activity. Since dCas12a can process its own gRNA from a polycistronic precursor/transcript, the orientation of the gRNA polycistronic precursor/transcript is in the antisense direction relative to the viral genome. This can result in generation of dsRNA which can trigger protein kinase R (PKR) activation and suppress translation in the packaging cells. Prevent PKR activation can be done by inclusion of VA-RNA expression vector in packing plasmid mixture. Alternatively, generation of PKR knockout packing cell line can be performed and used for packaging CRISPR-based components, e.g., CRISPRi or CRISPRn, as described herein.

In some embodiments, the dCas is a dCas12a. In some embodiments, the dCas12a is expressed using a single delivery vector. In some embodiments, the dCas12a and one or more gRNAs are expressed using a single delivery vector.

In some embodiments, the single delivery vector comprises the dCas12a and one or more gRNAs. In some embodiments, the one or more gRNAs are expressed as a polycistronic transcript/precursor. In some embodiments, the one or more gRNAs are designed to target a single gene. In some embodiments, the one or more gRNAs are designed to target different genes.

### Viral Concentration and Packaging

In order to generate virus for transfection of CRISPR-based components or RNAi to modulate target genes, packaging plasmids are utilized to aid viral generation. In some embodiments, packaging plasmids comprise lentiviral packaging plasmids. In some embodiments, the packaging plasmids comprise the 2^{nd} generation packaging plasmids. In some embodiments, the packaging plasmids comprise the 3^{rd} generation packaging plasmids.

In some embodiments, the 2^{nd} generation packaging plasmids comprise HIV transacting factors. In some embodiments, the packing plasmids comprises envelope protein expression vector. In some embodiments, the envelope protein expression vector comprises a plurality of envelope glycoproteins derived from Morbillivirus, Sendai virus (SeV), Nipah virus (NiV), Newcastle disease virus (NDV), or Baboon endogenous virus (BaEV), or a combination thereof. In some embodiments, the Morbillivirus is a measles virus (MV).

In some embodiments, the envelope protein expression vector comprises a VSV-G envelope protein. In some embodiments, the VSV-G envelope protein is fused to ZZ-type zinc finger domain (ZZ domain), which mediates binding to antibody Fc-domains. The ZZ domain peptide is an IgG Fc-binding peptide derived from protein A. This enables targeting of viral particles to target cell surface proteins mediated by an appropriate antibody that simultaneously binds the viral particle (via Fc domain) and the antibody target (via CDR). In some embodiments, the VSV-G envelope protein is co-expression with the ZZ domain.

In some embodiments, the VSV-G envelope protein is fused to biotin-binding domain. Fusion of the VSV-G envelope protein with biotin-binding domains can be used for biotinylated affinity reagents, e.g., antibodies. In some embodiments, the VSV-G envelope protein is co-expressed with biotin-binding domain.

In some embodiments, the packaging plasmids further comprise an expression vector of adenovirus-derived VA-RNAs. This expression vector of adenovirus-derived VA-RNAs can prevent PKR activation induced by dsRNA. Inhibition of PKR activation during viral packaging can increase the gene expression and effective viral packaging process.

Some cells, e.g., myeloid cells, dendritic cells, and macrophages, are refractory to lentiviral vector transduction due to the myeloid-specific restriction factor, SAMHD1. However, the SAMHD1-mediated restriction of viral transduction can be relieved by Vpx, which is a virion-packaged accessory protein that induces degradation of SAMHD1 upon infection. In some embodiments, the packing plasmids comprise a modified 2^{nd} generation packaging plasmid. In some embodiments, Vpx is incorporated into virions during viral particle production. In some embodiments, the modified 2^{nd} generation packaging plasmids comprises binding site for Vpx (derived from SIV-Gag) in Gag protein and expression vector encoding Vpx. In some embodiments, once Vpx is expressed in the packaging cells, Vpx bind to Gag protein and is co-packaged into viral particles. When released into infected cells, Vpx inhibits the protein SAMHD1.

Various transfection reagents and harvest medium for viral packaging can be used. In some embodiments, the transfection reagents comprise Lipofectamine 3000. In some embodiments, the transfection reagents comprise PEI. In some embodiments, the transfection reagents comprise liposomes. In some embodiments, the transfection reagents comprise TransIT. In some embodiments, the transfection reagents comprise nanoparticles. In some embodiments, the transfection reagents comprise cyclodextrins. In some embodiments, the transfection reagents comprise polymers. In some embodiments, the polymers are DEAE-dextran or polyethyleneimine. In some embodiments, the transfection reagents comprise dendrimers. In some embodiments, the transfection reagents comprise Fugene. In some embodiments, the transfection reagents utilize calcium phosphate precipitation. In some embodiments, electroporation is used for transfection. In some embodiments, microinjection is used for transfection. In some embodiments, a gene gun is used for transfection. In some embodiments, impalefection is used for transfection. In some embodiments, use of hydrostatic pressure is used for transfection. In some embodiments, continuous infusion is used for transfection. In some embodiments, sonication is used for transfection. In some embodiments, targeted ultrasound is used for transfection. In some embodiments, sonoporation is used for transfection. In some embodiments, optical methods using a focused laser is used for transfection. In some embodiments, biolistic particle delivery is used for transfection. In some embodiments, magnetofection is used for transfection.

In some embodiments, harvest medium comprises DMEM based medium. In some embodiments, the harvest medium comprises Advanced DMEM, HEPES, Fetal Calf Serum (FCS), MgCl₂, DNAse-I, or sodium-butyrate. In some embodiments, the HEPES is supplemented in the harvest medium at concentration of about 1 mM, about 10 mM, about 20 mM, about 30 mM, about 40 mM, about 50 mM, about 100 mM, or about 500 mM. In some embodiments, the FCS is supplemented in the harvest medium at concentration of about 0.1%, about 0.5%, about 1%, about 1.5%, about 2%, about 3%, about 5%, or about 10%. In some embodiments, the MgCl₂ is supplemented in the harvest medium at concentration of about 0.1 μM, about 0.5 μM, about 1 μM, about 2 μM, about 3 μM, about 4 μM, about 5 μM, about 6 μM, about 7 μM, about 8 μM, about 9 μM, about 10 μM, about 15 μM, about 20 μM, about 30 μM, about 50 μM, or about 100 μM. In some embodiments, the DNAse-I is supplemented in the harvest medium at concentration of about 0.1 U/mL, about 0.5 U/mL, about 1 U/mL, about 2 U/mL, about 3 U/mL, about 4 U/mL, about 5 U/mL, about 6 U/mL, about 7 U/mL, about 8 U/mL, about 9 U/mL, or about 10 U/mL. In some embodiments, the sodium-butyrate is supplemented in the harvest medium at concentration of about 0.1 μM, about 0.5 μM, about 1 μM, about 2 μM, about 3 μM, about 4 μM, about 5 μM, about 6 μM, about 7 μM, about 8 μM, about 9 μM, about 10 μM, about 15 μM, about 20 μM, about 30 μM, about 50 μM, or about 100 μM.

In some embodiments, the harvest medium further comprises cholesterol. In some embodiments, the cholesterol is complexed with methyl-beta-cyclodextrin. In some embodiments, the cholesterol is supplemented in the harvest medium at concentration of about 0.01 mM, about 0.02 mM, about 0.03 mM, about 0.04 mM, about 0.05 mM, about 0.06 mM, about 0.07 mM, about 0.08 mM, about 0.09 mM, about 0.10 mM, or about 1 mM. In some embodiments, the cholesterol complexed with methyl-beta-cyclodextrin is supplemented in the harvest medium at concentration of about 0.01 mM, about 0.02 mM, about 0.03 mM, about 0.04 mM, about 0.05 mM, about 0.06 mM, about 0.07 mM, about 0.08 mM, about 0.09 mM, about 0.10 mM, or about 1 mM.

In some embodiments, the harvest medium further comprises sorbitol. In some embodiments, the sorbitol is supplemented in the harvest medium at concentration of about 1 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, or about 300 mM.

In some embodiments, the harvest medium further comprises ataxia telangiectasia and Rad3-related protein kinase (ATR) inhibitor. In some embodiments, the ATR inhibitor is caffeine. In some embodiments, the caffeine is supplemented in the harvest medium at concentration of about 0.1 mM, about 1 mM, about 2 mM, about 3 mM, about 4 mM, about 5 mM, about 6 mM, about 7 mM, about 8 mM, about 9 mM, about 10 mM, about 15 mM, about 20 mM, about 30 mM, about 40 mM, or about 50 mM.

Packaging cells are used to package viral vector into a delivery vehicle, e.g., viral particle. In some embodiments, the packaging cells used to pack viral particles as described herein comprises 293T cells or modified cell lines or derivative cell lines from 293T cells. In some embodiments, the 293T cells are Lenti-X 293T (LX293T) cells.

In some embodiments, the 293T cells are seeded on culture plate coated with culture reagents. In some embodiments, the culture reagents comprise poly-L-Lysine, collagen, gelatin, Matrigel, or poly-D-Lysine. In some embodiments, gelatin is diluted to 0.1% in water. In some embodiments, the 293T cells are seeded on non-coated culture plate. In some embodiments, the poly-L-lysine is used at the final concentration of about 0.001%, about 0.002%, about 0.003%, about 0.004%, about 0.005%, about 0.006%, about 0.007%, about 0.008%, about 0.009%, about 0.010%, about 0.015%, or about 0.020%.

In some embodiments, after reverse transfection, the 293T cells are seeded at density of about 1×10⁶, about 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶ per 15-cm culture dish. In some embodiments, after reverse transfection, the 293T cells are seeded at density of at least 1×10⁶, 5×10⁶, 10×10⁶, 15×10⁶, 20×10⁶, 25×10⁶, 30×10⁶, 35×10⁶, 40×10⁶, 45×10⁶, 50×10⁶, 55×10⁶, 60×10⁶, 65×10⁶, 70×10⁶, 75×10⁶, 80×10⁶, 85×10⁶ per 15-cm culture dish.

In some embodiments, after reverse transfection, the LX293T cells are seeded at density of about 1×10⁶, about 5×10⁶, about 10×10⁶, about 15×10⁶, about 20×10⁶, about 25×10⁶, about 30×10⁶, about 35×10⁶, about 40×10⁶, about 45×10⁶, about 50×10⁶, about 55×10⁶, about 60×10⁶, about 65×10⁶, about 70×10⁶, about 75×10⁶, about 80×10⁶, about 85×10⁶ per 15-cm culture dish. In some embodiments, after reverse transfection, the LX293T cells are seeded at density of at least 1×10⁶, 5×10⁶, 10×10⁶, 15×10⁶, 20×10⁶, 25×10⁶, 30×10⁶, 35×10⁶, 40×10⁶, 45×10⁶, 50×10⁶, 55×10⁶, 60×10⁶, 65×10⁶, 70×10⁶, 75×10⁶, 80×10⁶, 85×10⁶ per 15-cm culture dish.

Viral particles can be concentrated during the collection of harvest medium. Using ultra-centrifugation of at least 25,000×g to collect viral particle can damage large viral genome, e.g., lentiviral particles, during the viral particle collection. In some embodiments, sucrose cushion is used during the collection of viral particles. In some embodiments, the viral particles are concentrated using high-speed centrifugation. In some embodiments, the high-speed centrifugation is used at least at 5,000×g, at last at 6,000×g, at least at 7,000×g, at least at 8,000×g, at least at 9,000×g, at least at 10,000×g, at least at 11,000×g, at least at 12,000×g, at least at 13,000×g, at least at 14,000×g, at least at 15,000×g, at least at 16,000×g, at least at 17,000×g, at least at 18,000×g, at least at 19,000×g, at least at 20,000×g, at least at 21,000×g, at least at 22,000×g, at least at 23,000×g, at least at 24,000×g, at least at 25,000×g, at least at 26,000×g, at least at 27,000×g, at least at 28,000×g, at least at 29,000×g, or at least at 30,000×g. In some embodiments, viral particles are collected using high-speed centrifugation of harvest medium at 10,000g through sucrose cushion.

Precipitation can be performed to increase viral concentration. In some embodiments, precipitation is performed using PEG, e.g., PEG 8000 or PEG 6000. In embodiments, the PEG is used at the final concentration of about 1%, about 5%, about 10%, about 15%, about 20%, about 25%, about 30%, about 35%, about 40% or about 45%, e.g., in PBS/saline. In embodiments, the PEG is used at the final concentration of at least 1%, at least 5%, at least 10%, at least 15%, at least 20%, at least 25%, at least 30%, at least 35%, at least 40% or at least 45%, e.g., in PBS/saline. In some instances, viral particles are incubated with PEG 8000 or PEG 6000 reagents for at least 6 hours. The viral concentration can be increased using centrifugation, for instance, at about 7,500×g. In some instance, centrifugation at 7,500×g is maintained for 90 minutes.

### Barcoding Strategies and Screening Method

The CRISPR gRNA or shRNA can be labeled with a barcode to allow identification of target gene during the screening process. Pooled screens utilize different barcoding strategies. Barcode sequences comprise unique nucleotide sequences that can be incorporated into the shRNA or CRISPR gRNA.

In CRISPR gRNA, internal barcode can be embedded within the gRNA, e.g., iBAR technology as disclosed in Zhu, S. et al. 2019. Genome Biology 20:20, which is herein incorporated by reference in its entirety. Briefly, a 6-nucleotide barcode sequence is embedded in the tetra loop of the gRNA scaffold. In some embodiments, the 6-nucleotide barcode sequence is embedded in the first stemloop of gRNA. This barcoding strategy enables to track an identical gRNA sequence in multiple independent constructs; thus, creating statistically independent bins for analysis. If using Twist Bioscience is used for library synthesis, iBAR barcode is included in the library design. The barcode is read out as part of read-1 during NGS and closely linked to the gRNA, which is also read out by read-1.

Alternatively, addition of random sequence labels (RSL) during library amplification can be utilized for barcoding strategies. This RSL strategy introduces random barcodes, about 10-nucleotide sequence, during the library amplification step. The oligonucleotide pool requires amplification by PCR before cloning into the gRNA delivery vector. RSL are introduced by the reverse primer during PCR. As many as 410 different RSL can be added to each gRNA oligo in the library. Each gRNA construct can be track at least 1 million times independently. RSLs can also be used to simply bin infection events for the same gRNA construct into 4, 16 or 64 bins (based on nucleotide in positions 1, 1+2 or 1+2+3) to enhance statistical analysis. RSLs are read out during NGS either as p7-index or as read-2, dependent on position in the library amplification primer relative to the p7-primer binding site co-introduced with the RSL. Since the barcode variations exceed the complexity of the libraries, repeated use of the same barcode with the same gRNA is rare. The number of distinct combinations of gRNA and RSL provides estimation of the total number of independent infection events (given the typically low number of infected cells in a given study—~ 100,000).

In some embodiments, one or more gRNAs further comprise internal barcode sequences. In some embodiments, the internal barcode sequence comprises at least 4 nucleotides, at least 5 nucleotides, at least 6 nucleotides, at least 7 nucleotides, at least 8 nucleotides, at least 9 nucleotides, at least 10 nucleotides, at least 11 nucleotides, or at least 12 nucleotides. In some embodiments, the internal barcode sequence is located upstream of gRNA. In some embodiments, the internal barcode sequence is located downstream of gRNA. In some embodiments, the internal barcode sequence is located within the gRNA as part of the gRNA sequence listed in Table 9A-Table 9D. In some embodiments, the internal barcode sequence is located in the tetra loop of the gRNA scaffold. In some embodiments, the internal barcode sequence is located in the first stemloop of gRNA.

In some embodiments, the gRNA delivery vector further comprises barcode sequences. In some embodiments, the barcode sequences comprise random sequence labels (RSL). In some embodiments, the RSL comprises at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, or at least 15 nucleotides. In some embodiments, the RSL is located in the same vector encoding gRNA. In some embodiments, the RSL is located upstream of the gRNA. In some embodiments, the RSL is located downstream of the gRNA.

In some embodiments, the gRNA delivery vector comprises a single gRNA. In some embodiments, the gRNA delivery vector comprises multiple gRNA(s). In some embodiments, the gRNA delivery vector comprises a single barcode sequence. In some embodiments, the gRNA delivery vector comprises multiple barcode sequences. In some embodiments, the gRNA delivery vector comprises combination of gRNA. In some embodiments, the gRNA delivery vector comprises concatenated barcodes.

In one aspect, provided are methods of evaluating a sample of cells for the presence, absence, and/or quantity of a nucleic acid sequence from the genetic pharmacopeia. The power of the genetic pharmacopeia becomes evident in the ability to read out effects on cell growth directly via ‘barcode’ counting of modified cells (e.g., transduced cancer cells). Cells harboring a gRNA or shRNA impairing cell viability will be less represented in the overall population (i.e. will ‘dropout’); this manifests as less frequent appearance of the gRNA/shRNA sequence itself within the overall population of guide/shRNA sequences. The method may employ next-generation sequencing (NGS), which is well-established, cost effective, commercial scale, robust, highly quantitative, and highly amenable to multiplexed analysis. Using sample index barcodes, sequence reads are demultiplexed and FASTQ files are mapped to a reference (FASTA format) of guide RNA sequences using Bowtie2 v2.4.51. Unmapped reads and secondary alignments are removed using Samtools v1.62. Guide RNA-level counts are generated by counting all primary read alignments using Pandas v1.4.23 via a custom script written in Python v3.9.134.

Sequencing can be performed with any appropriate sequencing technology, including but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLID sequencing. In some cases, next-generation sequencing involves high-throughput sequencing methods. Additional sequencing methods available to one of skill in the art may also be employed.

The resulting barcode distributions are interpreted to determine the effect of individual perturbations on the viability of a subject's cells. In some implementations, raw sequencing read counts are interpreted and remapped back into ‘drug space’. For instance, in the hypothetical case described above, if a particular gRNA was found to be less prevalent than expected within the population, this would suggest that the protein encoded by the gene target of the gRNA is required for the survival or proliferation of the patient's cancer cells. As such, the drug targeting that protein (identified in step 1 above) is suggested to be a potentially higher value therapeutic for the patient. Guide RNA-level counts are imported into R v4.2.15 for further processing and analysis using Tidyverse v1.3.16 packages. Low abundance guide RNAs (any guide containing<10 counts in any sample) are discarded. Count data are imported into CRISPRBetaBinomial v1.3.4 (CB2) and normalized through calculation of counts per million (CPM) values. The beta-binomial test (implemented in CB2) is then used to test for guide-level differential abundance between experimental groups. Guide-level P values are subsequently aggregated to the gene-level using Fisher's combined probability test. The Benjamini & Hochberg multiple hypothesis testing adjustment is used to estimate false discovery rate (FDR). One-sided FDR testing for the depletion of guide RNAs and logarithm of read count fold change comparing input and final timepoints are used to make gene dependency calls, with thresholds of FDR<=0.05 and log fold change <=−0.45 respectively. In some embodiments, use of methods of CRISPR-based gene modulation functional gene dependency determination are based on measuring sgRNA levels in a particular cell sample for one or more target genes measured by read counts assay from CRISPR-modified cells after a period of maintenance in culture. A mathematical model is used to assess gene dependency by comparing to non-dependent genes. Gene dependencies in these samples are defined as those genes without which the cells in the tumor sample experience a measurable fitness defect.

An exemplary method of evaluating the functional effect of genetically modifying cancer cells from a subject comprises: sequencing a plurality of modified cancer cells, wherein each modified cancer cell harbors one or more gene modulatory reagents, each gene modulatory reagent capable of knocking down or knocking out the function of a gene that encodes a protein target in a library of protein targets; and wherein a gene modulatory reagent that impairs cell viability will have fewer sequence reads than a gene modulatory reagent that does not impair cell viability. The method may further comprise determining which gene modulatory regents have fewer than a threshold number of sequence reads. The threshold number of sequence reads may be an expected number of sequence reads if the gene modulatory reagent did not impair cell viability. In some cases the threshold number of sequence reads is an average number of sequence reads for each gene modulatory reagent in the plurality of modified cancer cells. In some embodiments, the sequencing comprises sequencing genomic DNA extracted from cells of the cell sample following expression of CRISPRi gene modulatory reagents in the plurality of modified cancer cells. In some embodiments, genomic DNA extracted from a heterogeneous population of cells from the cell sample is sequenced to determine counts of sequencing reads of barcodes present in a library of CRISPRi gene modulatory reagents expressed in the plurality of modified cancer cells. In some embodiments, the heterogeneous population of cells from the cell sample comprises cancerous and non-cancerous cells. In some embodiments, the heterogeneous population of cells from the cell sample comprises cancerous cells with distinct functional gene dependencies for maintenance, growth, and/or proliferation in culture. In some embodiments, the amount of the one or more gene modulatory reagents that are delivered to cells of the cell sample is titrated to deliver on average a single distinct lentiviral vector to each cell of the cell sample that becomes a modified cancer cell following the successful delivery of the gene modulatory reagents. In some embodiments, the amount of the one or more gene modulatory reagents that are delivered to cells of the cell sample results in two or more distinct lentiviral vectors that are delivered to certain cells of the cell sample, wherein these cells of the cell sample are modified cancer cells comprising CRISPRi targeting reagents to target two or more genomic locations for transcriptional repression by RNA-guided targeting of a KRAB-dCas protein via distinct gRNA sequences. In some embodiments, some cells of the cell sample do not receive one or more gene modulatory reagents and remain unmodified cancer cells. In some embodiments, the sequencing comprises sequencing genomic DNA extracted from a plurality of cells from the cell sample, wherein the plurality of cells comprise modified cancer cells and unmodified cancer cells. In some embodiments, a sample of genomic DNA extracted from the plurality of cells from the cell sample comprises genomic DNA from at least about 10⁴, 5×10⁴, 10⁵, 5×10⁵, 10⁶, 5×10⁶, 10⁷, 5×10⁷, 10⁸, 5×10⁸, or 10⁹ cells. In some embodiments, at least about 20% of cells from the cell sample are cancer cells. In some embodiments, a sample of genomic DNA extracted from the plurality of cells from the cell sample comprises genomic DNA from at least about 10⁴, 5×10⁴, 10⁵, 5×10⁵, 10⁶, 5×10⁶, 10⁷, 5×10⁷, 10⁸, or 5×10⁸, cancer cells. In some embodiments, a sample of genomic DNA extracted from the plurality of cells from the cell sample comprises genomic DNA from at least about 10⁴, 5×10⁴, 10⁵, 5×10⁵, 10⁶, 5×10⁶, 10⁷, 5×10⁷, 10⁸, or 5×10⁸, modified cancer cells.

In some embodiments, the method further comprises correlating each gene modulatory reagent that has fewer than the threshold number of sequence reads to its corresponding protein target in the library of protein targets. The method may then also comprise correlating the corresponding protein target to a therapeutic molecule. The library of protein targets may comprise at least 1, at least 2, at least 3, at least 4, at least 5, at least 10, or at least 20 protein targets of TABLE 3. The library of protein targets may comprise at least 1, at least 2, at least 3, at least 4, at least 5, at least 10, or at least 20 protein targets of TABLE 4. The library of protein targets may comprise at least 1, at least 2, at least 3, at least 4, at least 5, at least 10, or at least 20 protein targets of TABLE 10A. The library of protein targets may comprise at least 1, at least 2, at least 3, at least 4, at least 5, at least 10, or at least 20 protein targets of TABLE 10B. The library of protein targets may comprise at least 1, at least 2, at least 3, at least 4, at least 5, at least 10, or at least 20 protein targets of TABLE 10C. In some embodiments, the method further comprises correlating each gene modulatory reagent derived from TABLE 9C containing gRNA sequences targeting kinase genes that has fewer than the threshold number of sequence reads to its corresponding protein target in the library of protein targets. The method may then also comprise correlating the corresponding protein target to a therapeutic molecule selected from TABLE 10C. In some embodiments, TABLE 10C lists exemplary therapeutic agents with associated kinase-related targets or gene products. In some embodiments, the method further comprises correlating each gene modulatory reagent derived from TABLE 9D containing gRNA sequences targeting IgSF genes that has fewer than the threshold number of sequence reads to its corresponding protein target in the library of protein targets. The method may then also comprise correlating the corresponding protein target to a therapeutic molecule selected from TABLE 10B. In some embodiments, TABLE 10B lists exemplary therapeutic agents with associated IgSF-related targets or gene products.

At least one of the one or more of the gene modulatory reagents may comprise a gRNA sequence comprising at least about 90% identity to a sequence selected from SEQ ID NOS: 55-1669. At least one of the one or more of the gene modulatory reagents may comprise a gRNA sequence comprising at least about 90% identity to a sequence selected from SEQ ID NOS: 55-459. At least one of the one or more of the gene modulatory reagents may comprise a gRNA sequence comprising at least about 90% or identity to a sequence selected from SEQ ID NOS: 460-1669. At least one of the one or more of the gene modulatory reagents may comprise a gRNA sequence comprising at least about 90% or identity to a sequence selected from SEQ ID NOS: 1670-8119. At least one of the one or more of the gene modulatory reagents may comprise a gRNA sequence comprising at least about 90% or identity to a sequence selected from SEQ ID NOS: 8120-12949.

In some embodiments, positive references are assayed for validation and Quality Control evaluation of gene modulation platform operation. In some embodiments, CRISPR-based methods include several sgRNAs targeting genes known to serve as core essential genes. These core essential genes serve as positive references. These positive reference target genes were selected according to depmap and literature as genes whose disruption impedes proliferation compared to wild-type cells. In some embodiments, these core essential genes serve as positive references for cancer cells, for normal cells, for healthy cells, for leukemia cells, for myeloid cells, for lymphoid cells, for acute myeloid leukemia cells, for acute lymphoid leukemia cells, for carcinoma cells, for sarcoma cells, for lymphoma cells, for cancer cell line cells, or any combination thereof. In some embodiments, core essential genes selected to serve as positive references for operation of a gene modulation platform are selected from: BIRC5, DNMT1, ERCC2, GART, KIF11, PSMB5, RAD51C, RAD51D, RPL3, SF3B1, SMARCB1, SRSF2, TOP2A, TUBG1, U2AF1, and ZRSR2. Positive identification of a positive reference gene as a gene dependency for maintenance, growth, and/or proliferation of cells from a cell sample in methods of a gene modulation platform serves to confirm the working operation of identification of functional gene dependencies in cells from the cell sample.

In some embodiments, negative references are assayed for validation and Quality Control evaluation of gene modulation platform operation. In some embodiments, non-targeting sgRNA (Controls) which are not expected to bind to any gene locus and therefore have no impact on cell behavior are include in operation of a CRISPR-based gene modulation platform. In some embodiments, selected non-impacting genes for cell types which are present in the cell sample are included as Controls in operation of a CRISPR-based gene modulation platform which are expected to have no impact on cell maintenance, growth, and/or proliferation of the cell type. In some embodiments, immune genes for T and B cells, such as CD3, CD4, and CD19 are included as negative Controls for assaying myeloid cells from a cell sample derived from a subject with AML. In some embodiments, absence of identification of one or more negative references as gene dependencies for maintenance, growth, and/or proliferation of cells from a cell sample in methods of a gene modulation platform serves to confirm the working operation of identification of functional gene dependencies in cells from the cell sample.

### Genetic Pharmacopeia

In one aspect, provided herein is a library of genetic elements which represent a collection of existing drugs for a particular disease or condition, e.g., hematological malignant cancer. These genetic elements are capable of modifying a patient's cells to mimic the effect of the existing drugs on the patient, allowing for personalized comprehensive functional profiling. The profiling may be performed in a pooled screening format to allow for screening of the effects of the modifications in parallel. Such highly parallel functional genomics methodology is utilized in preclinical biology, but has not been applicable to personalized therapeutic sensitivity profiling. Additionally, this approach enables comprehensive assessment of the impact of therapeutic manipulations in an in vivo testing paradigm, of critical importance for the reasons previously indicated herein.

Accordingly, disclosed herein are methods for the design, construction, and use of a genetic pharmacopeia comprising a plurality of gene modulatory reagents capable of modifying a patient's cells to knock out, or knock down, function of genes encoding for protein targets of a collection of existing drugs. In some embodiments, a genetic pharmacopeia is designed using publicly available tools, e.g., publicly available methods and reagents for gene editing or gene silencing. In some embodiments, a subset of these reagents will work poorly, most will be acceptable, and a minority will demonstrate exceptional performance. Pre-selection of reagents that have been validated to work well will be advantageous both with regard to efficiency of delivery and production of a more “compact” library, both of which reduce the number of patient-derived cells needed and increase the quality of data produced. In some embodiments, the design includes selection of the most efficacious or advantageous modulatory mechanism (e.g., CRISPRn, CRISPRi, RNAi). For CRISPR-based methods, the design comprises selection of the most advantageous RNA-guided endonuclease (e.g., Cas9 vs. Cas12a vs. Mad7). The design may also include selection of the most efficacious guide or seed sequences. The design may also include multiple gene modulatory reagents expressed from a single vector as a single or multiple transcriptional units. For instance, multiplexed gRNAs may be constructed for use with a Cas12 based nuclease (e.g., Cpf1) to generate a highly compact library. The design may also include elements in the library that allow for the identification, selection, or enrichment of transduced cells (e.g., fluorescent markers, antibiotic resistance cassettes, surface epitope expression cassettes).

The genetic pharmacopeia may be constructed in a format that is compatible with use in patient derived cells, e.g., primary cancer cells. In some embodiments, a viral delivery method is chosen for introduction of the gene modulatory reagent (e.g., guide or seed sequence). Non-limiting examples of viruses include lentivirus, adenovirus, adeno-associated virus, and other viruses disclosed herein. In some embodiments, a non-viral delivery method is selected. As a non-limiting example, the delivery method is transposase-mediated transposition. The library may be constructed using a combination of gene synthesis and pooled molecular cloning techniques. The library may be subject to quality control analysis to ensure full and approximately equal representation of the desired sequences. In some embodiments of a viral delivery method, pooled high-titer virus is prepared. In other embodiments, the virus is delivered in an array to facilitate an arrayed screening format.

### Library of Gene Modulatory Reagents

In one aspect, provided herein are libraries comprising a plurality of gene modulatory reagents, each gene modulatory reagent capable of knocking down or knocking out the function of a gene that encodes a protein target from a library of protein targets. The plurality of gene modulatory reagents may be capable of knocking down or knocking out the function of at least about 50%, at least about 60%, at least about 70%, at least about 80%, at least about 90%, or at least about 95% of the genes that encode for the protein targets in the library. In some cases, the library of protein targets comprises all known proteins targeted by known drugs capable of treating a particular disease or condition, e.g., hematological malignant cancers. In some cases, the library of protein targets comprises a plurality of kinase proteins targeted by drugs known to target one or more kinases or one or more kinase-regulated molecular pathways. In some embodiments, the library of gene modulatory reagents is selected to target one or more genes encoding a kinase. In some embodiments, the library of gene modulatory reagents is selected to target one or more IgSF genes. In some cases, the library of protein targets comprises a plurality of IgSF proteins targeted by drugs known to target one or more IgSF proteins or one or more IgSF-regulated molecular pathways.

In some embodiments, the library of gene modulatory reagents is capable of knocking down or knocking out the function of one or more genes encoding protein targets selected from TABLE 9A. In some cases, the protein targets comprise at least 1, at least 2, at least 3, at least 4, at least 5, at least 10, or at least 20 protein targets of TABLE 9A. In some embodiments, the library of gene modulatory reagents is capable of knocking down or knocking out the function of one or more genes encoding protein targets selected from TABLE 9B. In some cases, the protein targets comprise at least 1, at least 2, at least 3, at least 4, at least 5, at least 10, or at least 20 protein targets of TABLE 9B. In some embodiments, the library of gene modulatory reagents is capable of knocking down or knocking out the function of one or more genes encoding protein targets selected from TABLE 9C. In some cases, the protein targets comprise at least 1, at least 2, at least 3, at least 4, at least 5, at least 10, or at least 20 protein targets of TABLE 9C. In some embodiments, the library of gene modulatory reagents is capable of knocking down or knocking out the function of one or more genes encoding protein targets selected from TABLE 9D. In some cases, the protein targets comprise at least 1, at least 2, at least 3, at least 4, at least 5, at least 10, or at least 20 protein targets of TABLE 9D. In some cases, the library of gene modulatory reagents is capable of knocking down or knocking out the function of one or more genes encoding for protein targets of one or more known drugs selected from TABLES 3, 4, 10A, 10B, or 10C. In some cases, the one or more known drugs comprises at least 1, at least 2, at least 3, at least 4, at least 5, at least 10, or at least 20 drugs of TABLES 3, 4, 10A, 10B, or 10C. In some cases, the library of gene modulatory reagents is capable of knocking down or knocking out the function of one or more genes encoding for protein targets of one or more known drugs selected from TABLE 9A, TABLE 9B, TABLE 9C, or TABLE 9D. In some cases, the one or more known drugs comprises at least 1, at least 2, at least 3, at least 4, at least 5, at least 10, or at least 20 drugs of TABLES 3, 4, 10A, 10B, or 10C. The plurality of gene modulatory reagents may be capable of knocking down or knocking out the function of about 10 to about 2,000, from about 10 to about 500, from about 10 to about 200, from about 10 to about 150, from about 50 to about 500, from about 50 to about 200, about 50 to about 2,000, or about 100 to about 2,000 genes. The library may comprise about 10 to about 2,000, from about 10 to about 500, from about 10 to about 200, from about 10 to about 150, from about 50 to about 500, from about 50 to about 200, about 50 to about 2,000, or about 100 to about 2,000 gene modulatory reagents.

At least one of the gene modulatory reagents may be capable of knocking out the function of a gene. For instance, the at least one gene modulatory reagent is part of a CRISPR-based gene editing system. In some cases, one or more of the plurality of gene modulatory reagents each comprise a gRNA sequence comprising at least about 95% identity to a sequence selected from SEQ ID NOS: 55-1669. In some cases, one or more of the plurality of gene modulatory reagents each comprise a gRNA sequence comprising at least about 95% identity to a sequence selected from SEQ ID NOS: 55-459. In some cases, one or more of the plurality of gene modulatory reagents each comprise a gRNA sequence selected from SEQ ID NOS: 55-459. In some cases, one or more of the plurality of gene modulatory reagents each comprise a gRNA sequence comprising at least about 95% identity to a sequence selected from SEQ ID NOS: 460-1669. In some cases, one or more of the plurality of gene modulatory reagents each comprise a gRNA sequence selected from SEQ ID NOS: 460-1669. In some embodiments, gRNA sequences used in a CRISPR-based gene editing system are constructed to target one or more genes encoding a serine/threonine kinase. In some embodiments, gRNA sequences used in CRISPR-based gene editing system are constructed to target one or more genes encoding a tyrosine kinase. In some cases, one or more of the plurality of gene modulatory reagents each comprise a gRNA sequence comprising at least about 95% identity to a sequence selected from SEQ ID NOS: 1670-8119. In some cases, one or more of the plurality of gene modulatory reagents each comprise a gRNA sequence selected from SEQ ID NOS: 1670-8119. In some embodiments, gRNA sequences used in a CRISPR-based gene editing system are constructed to target one or more genes encoding IgSF family members. In some cases, one or more of the plurality of gene modulatory reagents each comprise a gRNA sequence comprising at least about 95% identity to a sequence selected from SEQ ID NOS: 8120-12949. In some cases, one or more of the plurality of gene modulatory reagents each comprise a gRNA sequence selected from SEQ ID NOS: 8120-12949. In some cases, at least one of the gene modulatory reagents comprise a gRNA sequence having homology to at least a portion of the gene whose function is knocked out by the gene modulatory reagent. In some embodiments, the gene modulatory reagents comprise one or more control sequences. As a non-limiting example, the sequence is a gRNA control that does not have a target in the human genome.

At least one of the gene modulatory reagents may be capable of knocking down the function of a gene. For instance, the at least one gene modulatory reagent comprises an shRNA sequence having homology to at least a portion of the gene whose function is knocked down by the gene modulatory reagent. The homology may be at least about 90% sequence identity. The at least a portion may be at least about 15 contiguous nucleotides.

In some embodiments, a library of gene modulatory reagents comprises one or more gene modulatory reagents that target a gene of TABLE 9A. In some embodiments, the library comprises one or more gene modulatory reagents that target at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, or all of the gene targets of TABLE 9A. In some embodiments, the library comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at at least 45, at least 50, at least 55, at least 60, at least 65, at least 70, at least 75, at least 80, at least 85, at least 90, or all of the gRNA of TABLE 9A.

In some embodiments, a library of gene modulatory reagents comprises one or more gene modulatory reagents that target a gene of TABLE 9B. In some embodiments, the library comprises one or more gene modulatory reagents that target at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, or all of the gene targets of TABLE 9B. In some embodiments, the library comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, or all of the gRNA of TABLE 9B.

In some embodiments, a library of gene modulatory reagents comprises one or more gene modulatory reagents that target a gene of TABLE 9C. In some embodiments, the library comprises one or more gene modulatory reagents that target at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, or all of the gene targets of TABLE 9C. In some embodiments, the library comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, or all of the gRNA of TABLE 9C.

In some embodiments, a library of gene modulatory reagents comprises one or more gene modulatory reagents that target a gene of TABLE 9D. In some embodiments, the library comprises one or more gene modulatory reagents that target at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, or all of the gene targets of TABLE 9D. In some embodiments, the library comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at at least 45, at least 50, at least 55, at least 60, at least 65, at least 70, at least 75, at least 80, at least 85, at least 90, or all of the gRNA of TABLE 9D.

In some embodiments, one or more of the gene modulatory reagents is designed to knock out or knock down the function of a positive control gene, such as a core essential gene for the cell. Such reagents may serve as a positive control for library functionality. In some embodiments, one or more of the gene modulatory reagents is designed to knock out or knock down the function of a non-targeting gene and/or a targeting and non-genic gene. Such gene modulatory reagents may serve as negative controls.

In some embodiments, one or more of the gene modulatory reagents is positioned within a vector. The vector may comprise an adapter sequence. The adapter sequence may comprise a type IIS restriction enzyme cleavage site, which may allow for GoldenGate assembly cloning. The adapter sequence may comprise homology arms compatible with a destination vector allowing for cloning by overhang homology based methods, such as Gibson assembly. The vector may also comprise genetic elements of a virus. Non-limiting examples of viruses include, but are not limited to, adenovirus, retrovirus, adeno-associated virus (AAV), pox virus, parvovirus, baculovirus, measles virus, herpes simplex virus (HSV), Moloney Murine Leukemia Virus (MoMLV, MMLV, MuLV, or MLV), Murine Stem cell Virus (MSCV), and human immunodeficiency virus (HIV). The vector may also comprise a sequence encoding a marker, an antibiotic resistance cassette, or surface epitope expression cassette, or a combination thereof. The marker may be a fluorescent marker.

### Therapeutic Molecules

Methods described herein may be used to select a therapeutic molecule or a plurality of therapeutic molecules which target a particular protein or a particular molecular pathway that has been functionally validated to impact cancer cell survival, growth, and/or proliferation for a specific cancer from a subject. In one aspect, provided herein is a pharmacologic landscape comprising a compilation of therapeutic agents having known protein targets, referred to as a drug compilation. The drug compilation may include low molecular weight drugs (e.g., having a molecule weight less than about 1 kDa) and biologic drugs (e.g., proteins such as antibodies). The drug compilation may comprise drugs suitable for a patient's particular disease or condition, e.g., hematological malignant cancers. In various embodiments, the drug compilation includes FDA-approved therapeutic agents and as such may be expanded as new drugs are developed. The drug compilation may include all or nearly all of the targeted drugs treating a particular class of disease, e.g., the drug compilation includes at least about 50%, at least about 60%, at least about 70%, at least about 80%, at least about 90%, or 100% of known FDA-approved drugs for a particular disease class having a known protein target. Also provided herein are compilations of a particular target classes of interest (e.g., G-protein coupled receptors, kinases, etc.).

Selected therapeutic molecules can be administered by a variety of methods to the subject in need thereof. In some embodiments, the selected therapeutic molecule or molecules are administered orally. In some embodiments, the selected therapeutic molecule or molecules are administered by injection. In some embodiments, the selected therapeutic molecule or molecules are administered intravenously. In some embodiments, the selected therapeutic molecule or molecules are administered nasally. In some embodiments, the selected therapeutic molecule or molecules are administered through inhalation. In some embodiments, the selected therapeutic molecule or molecules are administered buccally. In some embodiments, the selected therapeutic molecule or molecules are administered topically. In some embodiments, dosages of the selected therapeutic molecule or molecules are determined to administer a therapeutically effective dosage. In some embodiments, dosages of the selected therapeutic molecule or molecules are administered as part of a treatment regimen. In some embodiments, a clinical evaluation of the subject may occur during the course of a treatment regimen. In some embodiments, the clinical evaluation may indicate a need to revise a treatment dosage. In some embodiments, the clinical evaluation may indicate a need to revise a treatment administration frequency. In some embodiments, the clinical evaluation may indicate a need to change to the administration of a different selected therapeutic molecule or molecules. In some embodiments, the clinical evaluation may indicate a need to cease a treatment regimen. In some embodiments, the clinical evaluation may indicate a need to restart a treatment regimen.

### Methods of Treatment

Further provided herein are methods of treating a subject having a disease or condition, wherein the subject has been determined to be susceptible to a targeted therapeutic agent using a method described herein. In some cases, the disease or condition is cancer. In some embodiments, the cancer comprises a carcinoma. In some embodiments, the cancer comprises a sarcoma. In some embodiments, the cancer maintains an active p53-dependent DDR. In some embodiments, the cancer comprises a hematologic malignancy. In some embodiments, the hematological malignancy comprises a myeloid neoplasm, lymphoid neoplasm, or histiocytic and dendritic cell neoplasm.

In some embodiments, the cancer of the subject comprises a hematologic malignancy. In some embodiments, the hematologic malignancy comprises a leukemia, a lymphoma, a myeloma, or a combination thereof. In some embodiments, the leukemia comprises Acute Myeloid Leukemia (AML).

In some embodiments, the myeloid neoplasms comprise a condition listed in TABLE 5. In some embodiments, the myeloid neoplasms comprise myeloproliferative neoplasms, mastocytosis, myeloid/lymphoid neoplasms with eosinophilia and gene rearrangement, myelodysplastic/myeloproliferative neoplasms, myelodysplastic syndromes, myeloid neoplasms with germline predisposition, acute myeloid leukemia and related neoplasms, blastic plasmacytoid dendritic cell neoplasm, or acute leukemias of ambiguous lineage.

In some embodiments, the myeloproliferative neoplasms comprise chronic myeloid leukemia, BCR-ABL1-positive, chronic neutrophilic leukemia, polycythemia vera, primary myelofibrosis, essential thrombocythemia, chronic eosinophilic leukemia, not otherwise specified, or myeloproliferative neoplasm, unclassifiable.

In some embodiments, the mastocytosis comprises cutaneous mastocytosis, systemic mastocytosis, or mast cell sarcoma. In some embodiments, the systemic mastocytosis comprises indolent systemic mastocytosis, smoldering systemic mastocytosis, systemic mastocytosis with an associated hematological neoplasm, aggressive systemic mastocytosis, or mast cell leukemia.

In some embodiments, the myeloid/lymphoid neoplasms with eosinophilia and gene rearrangement comprises myeloid/lymphoid neoplasms with PDGFRA rearrangement, myeloid/lymphoid neoplasms with PDGFRB rearrangement, myeloid/lymphoid neoplasms with FGFR1 rearrangement, or myeloid/lymphoid neoplasms with PCM1-JAK2 (provisional entity).

In some embodiments, the myelodysplastic/myeloproliferative neoplasms comprise chronic myelomonocytic leukemia, atypical chronic myeloid leukemia, BCR-ABL1-negative, juvenile myelomonocytic leukemia, myelodysplastic/myeloproliferative neoplasm with ring sideroblasts and thrombocytosis, or myelodysplastic/myeloproliferative neoplasm, unclassifiable.

In some embodiments, the myelodysplastic syndromes comprise myelodysplastic syndrome with single lineage dysplasia, myelodysplastic syndrome with multilineage dysplasia, myelodysplastic syndrome with ring sideroblasts, myelodysplastic syndrome with excess blasts, myelodysplastic syndrome with isolated del (5q), myelodysplastic syndrome, unclassifiable, or refractory cytopenia of childhood (provisional entity). In some embodiments, the myelodysplastic syndrome with ring sideroblasts comprises myelodysplastic syndrome with ring sideroblasts and single lineage dysplasia, or myelodysplastic syndrome with ring sideroblasts and multilineage dysplasia.

In some embodiments, the myeloid neoplasms with germline predisposition comprises myeloid neoplasms with germline predisposition without a preexisting disorder or organ dysfunction, myeloid neoplasms with germline predisposition and preexisting platelet disorders, or myeloid neoplasms with germline predisposition and other organ dysfunction. In some embodiments, the myeloid neoplasms with germline predisposition without a preexisting disorder or organ dysfunction comprises acute myeloid leukemia with germline CEBPA mutation, or myeloid neoplasms with germline DDX41 mutation. In some embodiments, the myeloid neoplasms with germline predisposition and preexisting platelet disorders comprises myeloid neoplasms with germline RUNX1 mutation, myeloid neoplasms with germline ANKRD26 mutation, or myeloid neoplasms with germline ETV6 mutation. In some embodiments, the myeloid neoplasms with germline predisposition and other organ dysfunction comprises myeloid neoplasms with germline GATA2 mutation, myeloid neoplasms associated with bone marrow failure syndromes, myeloid neoplasms associated with telomere biology disorders, juvenile myelomonocytic leukemia associated with neurofibromatosis, Noonan syndrome or Noonan syndrome-like disorders, or myeloid neoplasms associated with Down syndrome.

In some embodiments, the acute myeloid leukemia and related neoplasms comprises acute myeloid leukemia with recurrent genetic abnormalities, acute myeloid leukemia with myelodysplasia-related changes, therapy-related myeloid neoplasms, acute myeloid leukemia, not otherwise specified, myeloid sarcoma, or myeloid proliferations related to Down syndrome. In some embodiments, the acute myeloid leukemia with recurrent genetic abnormalities comprises acute myeloid leukemia with t (8;21) (q22;q22.1); RUNX1-RUNXITI, acute myeloid leukemia with inv (16) (p13.1q22) ort (16;16) (p13.1;q22); CBFB-MYH11, acute promyelocytic leukemia with PML-RARA, acute myeloid leukemia with t (9;11) (p21.3;q23.3); MLLT3-KMT2A, acute myeloid leukemia with t (6;9) (p23;q34.1); DEK-NUP214, acute myeloid leukemia with inv (3) (q21.3q26.2) or t (3;3) (q21.3;q26.2); GATA2, MECOM, acute myeloid leukemia (megakaryoblastic) with t (1;22) (p13.3;q13.3); RBM15-MKL1, acute myeloid leukemia with BCR-ABL1 (provisional entity), acute myeloid leukemia with mutated NPM1, acute myeloid leukemia with biallelic mutations of CEBPA, or acute myeloid leukemia with mutated RUNX1 (provisional entity).

In some embodiments, the acute myeloid leukemia, not otherwise specified comprises acute myeloid leukemia with minimal differentiation, acute myeloid leukemia without maturation, acute myeloid leukemia with maturation, acute myelomonocytic leukemia, acute monoblastic/monocytic leukemia, pure erythroid leukemia, acute megakaryoblastic leukemia, acute basophilic leukemia, or acute panmyelosis with myelofibrosis.

In some embodiments, the myeloid proliferations related to Down syndrome comprises transient abnormal myelopoiesis or myeloid leukemia associated with Down syndrome.

In some embodiments, the acute leukemias of ambiguous lineage comprises acute undifferentiated leukemia, mixed phenotype acute leukemia with t (9;22) (q34.1;q11.2); BCR-ABL1, mixed phenotype acute leukemia with t (v;11q23.3); KMT2A rearranged, mixed phenotype acute leukemia, B/myeloid, not otherwise specified, or mixed phenotype acute leukemia, T/myeloid, not otherwise specified.

In some embodiments, the lymphoid neoplasms comprise a condition listed in TABLE 6. In some embodiments, the lymphoid neoplasms comprise precursor lymphoid neoplasms, mature B-cell neoplasms, mature T- and NK-cell neoplasms, Hodgkin lymphoma, or posttransplant lymphoproliferative disorders (PTLD).

In some embodiments, the precursor lymphoid neoplasms comprise B-lymphoblastic leukemia/lymphoma, T-lymphoblastic leukemia/lymphoma, or natural killer (NK) cell lymphoblastic leukemia/lymphoma (provisional entity).

In some embodiments, the B-lymphoblastic leukemia/lymphoma comprises B-lymphoblastic leukemia/lymphoma, not otherwise specified or B-lymphoblastic leukemia/lymphoma with recurrent genetic abnormalities. In some embodiments, the B-lymphoblastic leukemia/lymphoma with recurrent genetic abnormalities comprises B-lymphoblastic leukemia/lymphoma with t (9;22) (q34.1;q11.2); BCR-ABL1, B-lymphoblastic leukemia/lymphoma with t (v;11q23.3); KMT2A rearranged, B-lymphoblastic leukemia/lymphoma with t (12;21) (p13.2;q22.1); ETV6-RUNX1, B-lymphoblastic leukemia/lymphoma with hyperdiploidy, B-lymphoblastic leukemia/lymphoma with hypodiploidy, B-lymphoblastic leukemia/lymphoma with t (5;14) (q31.1;q32.3); IL3-IGH, B-lymphoblastic leukemia/lymphoma with t (1;19) (q23;p13.3); TCF3-PBX1, B-lymphoblastic leukemia/lymphoma, BCR-ABL1-like (provisional entity), or B-lymphoblastic leukemia/lymphoma with iAMP21 (provisional entity).

In some embodiments, the T-lymphoblastic leukemia/lymphoma comprises early T-cell precursor lymphoblastic leukemia (provisional entity).

In some embodiments, the mature B-cell neoplasms comprises chronic lymphocytic leukemia/small lymphocytic lymphoma, monoclonal B-cell lymphocytosis, B-cell prolymphocytic leukemia, splenic marginal zone lymphoma, hairy cell leukemia, splenic B-cell lymphoma/leukemia, unclassifiable, lymphoplasmacytic lymphoma, IgM monoclonal gammopathy of undetermined significance, heavy-chain diseases, plasma cell neoplasms, extranodal marginal zone lymphoma of mucosa-associated lymphoid tissue (MALT lymphoma), nodal marginal zone lymphoma, follicular lymphoma, pediatric-type follicular lymphoma, large B-cell lymphoma with IRF4 rearrangement (provisional entity), primary cutaneous follicle center lymphoma, mantle cell lymphoma, diffuse large B-cell lymphoma, not otherwise specified, T-cell/histiocyte-rich large B-cell lymphoma, primary diffuse large B-cell lymphoma of the central nervous system, primary cutaneous diffuse large B-cell lymphoma, leg type, EBV-positive diffuse large B-cell lymphoma, not otherwise specified, EBV-positive mucocutaneous ulcer (provisional entity), diffuse large B-cell lymphoma associated with chronic inflammation, lymphomatoid granulomatosis, primary mediastinal (thymic) large B-cell lymphoma, intravascular large B-cell lymphoma, ALK-positive large B-cell lymphoma, plasmablastic lymphoma, primary effusion lymphoma, HHV8-associated lymphoproliferative disorders, Burkitt lymphoma, Burkitt-like lymphoma with 11q aberration (provisional entity), high-grade B-cell lymphoma, or B-cell lymphoma, unclassifiable, with features intermediate between diffuse large B-cell lymphoma and classical Hodgkin lymphoma.

In some embodiments, the splenic B-cell lymphoma/leukemia, unclassifiable comprises splenic diffuse red pulp small B-cell lymphoma (provisional entity) or hairy cell leukemia-variant (provisional entity). In some embodiments, the lymphoplasmacytic lymphoma comprises Waldenström macroglobulinemia.

In some embodiments, the heavy-chain diseases comprise μ heavy-chain disease, γ heavy-chain disease, or a heavy-chain disease.

In some embodiments, the plasma cell neoplasms comprise non-IgM monoclonal gammopathy of undetermined significance (IgG/A), plasma cell myeloma (a.k.a. multiple myeloma), plasma cell myeloma variants, plasmacytoma, monoclonal immunoglobulin deposition diseases, or plasma cell neoplasms with associated paraneoplastic syndrome. In some embodiments, the plasma cell myeloma variants comprise smoldering (asymptomatic) plasma cell myeloma, non-secretory myeloma, or plasma cell leukemia. In some embodiments, the plasmacytoma comprises solitary plasmacytoma of bone or extraosseous plasmacytoma. In some embodiments, the monoclonal immunoglobulin deposition diseases comprise primary amyloidosis or light chain and heavy chain deposition diseases. In some embodiments, the plasma cell neoplasms with associated paraneoplastic syndrome comprises POEMS syndrome or TEMPI syndrome.

In some embodiments, the nodal marginal zone lymphoma comprises pediatric nodal marginal zone lymphoma (provisional entity).

In some embodiments, the follicular lymphoma comprises testicular follicular lymphoma, in situ follicular neoplasia, or duodenal-type follicular lymphoma.

In some embodiments, the mantle cell lymphoma comprises leukemic non-nodal mantle cell lymphoma or in situ mantle cell neoplasia.

In some embodiments, the diffuse large B-cell lymphoma, not otherwise specified comprises germinal center B-cell type or activated B-cell type.

In some embodiments, the diffuse large B-cell lymphoma associated with chronic inflammation comprises fibrin-associated diffuse large B-cell lymphoma.

In some embodiments, the HHV8-associated lymphoproliferative disorders comprises multicentric Castleman disease, HHV8-positive diffuse large B-cell lymphoma, not otherwise specified (provisional entity), or HHV8-positive germinotropic lymphoproliferative disorder.

In some embodiments, the high-grade B-cell lymphoma comprises high-grade B-cell lymphoma with MYC and BCL2 and/or BCL6 rearrangements or high-grade B-cell lymphoma, not otherwise specified.

In some embodiments, the mature T- and NK-cell neoplasms comprises T-cell prolymphocytic leukemia, T-cell large granular lymphocytic leukemia, chronic lymphoproliferative disorder of NK cells (provisional entity), aggressive NK-cell leukemia, EBV-positive T-cell and NK-cell lymphoproliferative diseases of childhood, adult T-cell leukemia/lymphoma, extranodal NK-/T-cell lymphoma, nasal type, intestinal T-cell lymphoma, hepatosplenic T-cell lymphoma, subcutaneous panniculitis-like T-cell lymphoma, mycosis fungoides, Sézary syndrome, primary cutaneous CD30+ T-cell lymphoproliferative disorders, primary cutanoue peripheral T-cell lymphomas, rare subtypes, peripheral T-cell lymphoma, not otherwise specified, angioimmunoblastic T-cell lymphoma and other nodal lymphomas of T follicular helper cell origin, anaplastic large-cell lymphoma, or breast implant-associated anaplastic large-cell lymphoma (provisional entity).

In some embodiments, the EBV-positive T-cell and NK-cell lymphoproliferative diseases of childhood comprises systemic EBV-positive T-cell lymphoma of childhood, chronic active EBV infection of T- and NK-cell type, systemic form, hydroa vacciniforme-like lymphoproliferative disorder, or severe mosquito bite allergy.

In some embodiments, the intestinal T-cell lymphoma comprises enteropathy-associated T-cell lymphoma, monomorphic epitheliotropic intestinal T-cell lymphoma, intestinal T-cell lymphoma, not otherwise specified, or indolent T-cell lymphoproliferative disorder of the gastrointestinal tract (provisional entity).

In some embodiments, the primary cutaneous CD30+ T-cell lymphoproliferative disorders comprises lymphomatoid papulosis or primary cutaneous anaplastic large cell lymphoma.

In some embodiments, the primary cutanoue peripheral T-cell lymphomas, rare subtypes comprises primary cutaneous γδ T-cell lymphoma, primary cutaneous CD8+ aggressive epidermotropic cytotoxic T-cell lymphoma (provisional entity), primary cutaneous acral CD8+ T-cell lymphoma (provisional entity), or primary cutaneous CD4+ small/medium T-cell lymphoproliferative disorder (provisional entity).

In some embodiments, the angioimmunoblastic T-cell lymphoma and other nodal lymphomas of T follicular helper cell origin comprises angioimmunoblastic T-cell lymphoma, follicular T-cell lymphoma (provisional entity), or nodal peripheral T-cell lymphoma with T follicular helper phenotype (provisional entity).

In some embodiments, the anaplastic large-cell lymphoma comprises anaplastic large-cell lymphoma, ALK-positive or anaplastic large-cell lymphoma, ALK-negative.

In some embodiments, the Hodgkin lymphoma comprises nodular lymphocyte predominant Hodgkin lymphoma or classical Hodgkin lymphoma. In some embodiments, the classical Hodgkin lymphoma comprises nodular sclerosis classical Hodgkin lymphoma, lymphocyte-rich classical Hodgkin lymphoma, mixed cellularity classical Hodgkin lymphoma, or lymphocyte-depleted classical Hodgkin lymphoma.

In some embodiments, the posttransplant lymphoproliferative disorders (PTLD) comprises plasmacytic hyperplasia PTLD, infectious mononucleosis PTLD, florid follicular hyperplasia PTLD, polymorphic PTLD, monomorphic PTLD, or classical Hodgkin lymphoma PTLD.

In some embodiments, the monomorphic PTLD comprises monomorphic B-cell PTLD or monomorphic T/NK-cell PTLD.

In some embodiments, the histiocytic and dendritic cell neoplasms comprise a condition listed in TABLE 7. In some embodiments, the histiocytic and dendritic cell neoplasms comprise histiocytic sarcoma, tumors derived from Langerhans cells, indeterminate dendritic cell tumor, interdigitating dendritic cell sarcoma, follicular dendritic cell sarcoma, fibroblastic reticular cell tumor, disseminated juvenile xanthogranuloma, or Erdheim-Chester disease. In some embodiments, the tumors derived from Langerhans cells comprise Langerhans cell histiocytosis or Langerhans cell sarcoma. In some embodiments, the follicular dendritic cell sarcoma comprises inflammatory pseudotumor-like follicular/fibroblastic dendritic cell sarcoma. In some embodiments, exemplary different classes and subtypes of heme malignancies are listed in TABLE 8.

In one aspect, the present disclosure provides a method of treating a subject having cancer with a targeted cancer therapeutic, the method comprising: identifying the subject as having a cancer susceptible to treatment with the targeted cancer therapeutic, and administering to the subject the targeted cancer therapeutic; wherein the cancer of the subject is susceptible to treatment if proliferation and/or viability of cancer cells isolated from the subject is reduced and/or inhibited after (i) knocking down the function of the target of the targeted cancer therapeutic or (ii) knocking out the target of the targeted cancer therapeutic. In some embodiments, determining whether the subject has a cancer susceptible to treatment is performed before treating the subject. In some embodiments, the cancer is susceptible to treatment if proliferation and/or viability of cancer cells isolated from the subject is reduced and/or inhibited after knocking down the function of the target of the targeted cancer therapeutic. In some embodiments, subjects that have been treated for a cancer are assessed to determine if they are considered to have exhibited complete response (CR) to treatment without molecular residual disease (MRD), CR to treatment with MRD, partial response (PR), non-responders with MRD, or non-responders with refractory disease. In some embodiments, determining whether the subject has a cancer that is susceptible to treatment is performed during or immediately following completion of a treatment regimen of the subject. In some embodiments, immediately following completion of a treatment regimen is a time within 2 weeks after administration of a final dosage of therapeutic from the treatment regimen. In some embodiments, a biopsy (e.g., a bone marrow aspirate) or sample collection (e.g., peripheral blood draw) is performed prior to initiation of a treatment regimen. In some embodiments, a biopsy (e.g., a bone marrow aspirate) or sample collection (e.g., peripheral blood draw) is performed during or immediately following completion of a treatment regimen. In some embodiments, cells from a cell sample collected during or immediately following completion of a treatment regimen are analyzed and compared to results from cells from a cell sample collected prior to initiation of a treatment regimen to determine a change in functional gene dependency of cancer cells during or following the treatment regimen. In some embodiments, the treatment regimen specifies dosage, schedule, and duration of the treatment. In some embodiments, identifying the subject as having a cancer susceptible to a treatment with a targeted cancer therapeutic that targets the determined functional gene dependency in cells from the cell sample is used to direct cancer treatment in the subject to a new treatment regimen comprising administering a targeted cancer therapeutic to the identified gene dependency. In some embodiments, identifying the subject as having a cancer susceptible to a treatment with one or more targeted cancer therapeutics that target the one or more determined functional gene dependencies in cells from the cell sample is used to direct cancer treatment in the subject to a new treatment regimen comprising administering one or more targeted cancer therapeutics to the identified gene dependency or dependencies. In some embodiments, the NGS is used to identify oncogenic or tumor suppressor mutations in cancer cells from the subject. In some embodiments, confirmation of one or more oncogenic or tumor suppressor mutations in cancer cells from the subject is combined with the determination of functional gene dependency or dependencies in cells from the cell sample to direct cancer treatment in the subject to a new treatment regimen comprising administering one or more targeted cancer therapeutics.

In some embodiments, methods comprising: identifying the subject as susceptible to treatment with the targeted cancer therapeutic, and administering to the subject the targeted cancer therapeutic; wherein the subject is susceptible to treatment if proliferation and/or viability of cancer cells isolated from the subject is reduced and/or inhibited after (i) knocking down the function of the target of the targeted cancer therapeutic or (ii) knocking out the target of the targeted cancer therapeutic are performed either before or after the subject has been determined to have exhibited complete response (CR) to treatment without molecular residual disease (MRD), CR to treatment with MRD, non-responders with MRD, or non-responders with refractory disease. In some embodiments, the methods analyze a plurality of samples for the subject and the plurality of samples may be taken before treatment initiation, during a period of treatment, or following completion of treatment. In some embodiments, the conclusions of predicted gene dependencies and selection of targeted cancer therapeutic are different for cell samples obtained before treatment initiation, during a period of treatment, or following completion of treatment. In some embodiments, assaying samples during a period of treatment or following completion of treatment may inform a decision to switch treatment in the subject to a more effective targeted cancer therapeutic.

In some cases, prior to sequencing, the plurality of modified cancer cells has been propagated. Propagation may comprise maintenance of the modified cancer cells in a 2D in vitro culture. Propagation may comprise maintenance of the modified cancer cells in a 3D in vitro culture. Propagation may comprise maintenance of the modified cancer cells in vivo. Propagation may occur within an animal model, e.g., where the animal is a rodent.

In some embodiments, the cancer cells contacted with the library of gene modulatory reagents are primary cancer cells. Contacting may comprise introducing the one or more gene modulatory reagents into a plurality of cancer cells by a viral or non-viral delivery method. Each of the gene modulatory reagents in the library may be encoded on a viral vector. In some embodiments, some cells of the cell sample do not receive the one or more gene modulatory reagents. In some embodiments, individual cells of the cell sample do receive at least one gene modulatory reagent. In some embodiments, individual cells of the cell sample receive one or gene modulatory reagent. In some embodiments, cells receiving at least one of the one or gene modulatory reagents become modified cancer cells.

In non-limiting embodiments, the viral vector comprises a lentiviral vector, adenoviral vector, or adeno-associated viral vector. An exemplary non-viral delivery method comprises transposase-mediated transposition.

In some embodiments, the library of therapeutic molecules comprises at least 1, at least 2, at least 3, at least 4, at least 5, at least 10, or at least 20 therapeutic agents of TABLE 3. In some embodiments, the library of therapeutic molecules comprises at least 1, at least 2, at least 3, at least 4, at least 5, at least 10, or at least 20 therapeutic agents of TABLE 4. In some embodiments, the library of therapeutic molecules comprises at least 1, at least 2, at least 3, at least 4, at least 5, at least 10, or at least 20 therapeutic agents of TABLE 10A. In some embodiments, the library of therapeutic molecules comprises at least 1, at least 2, at least 3, at least 4, at least 5, at least 10, or at least 20 therapeutic agents of TABLE 10B. In some embodiments, the library of therapeutic molecules comprises at least 1, at least 2, at least 3, at least 4, at least 5, at least 10, or at least 20 therapeutic agents of TABLE 10C. In some embodiments, the library of gene modulatory reagents comprises from about 10 to about 2,000, from about 10 to about 500, from about 10 to about 200, from about 10 to about 150, from about 50 to about 500, from about 50 to about 200, from about 50 to about 2,000, from about 100 to about 2,000, or from about 500 to about 2,000 different gene modulatory reagents. In some embodiments, one or more gene modulatory reagents from the library of gene modulatory reagents comprise a nucleic acid sequence at least about 80%, 85%, 90%, 91%, 92%, 93%, 94%, 95%, or 100% identical to at least about 15 contiguous nucleotides of a gene encoding a protein of TABLE 9A. In some embodiments, one or more gene modulatory reagents from the library of gene modulatory reagents comprise a nucleic acid sequence identical to at least about 15 contiguous nucleotides of a gene encoding a protein of TABLE 9A. In some embodiments, one or more gene modulatory reagents from the library of gene modulatory reagents comprise a nucleic acid sequence at least about 80%, 85%, 90%, 91%, 92%, 93%, 94%, 95%, or 100% identical to at least about 15 contiguous nucleotides of a gene encoding a protein of TABLE 9B. In some embodiments, one or more gene modulatory reagents from the library of gene modulatory reagents comprise a nucleic acid sequence identical to at least about 15 contiguous nucleotides of a gene encoding a protein of TABLE 9B. In some embodiments, one or more gene modulatory reagents from the library of gene modulatory reagents comprise a nucleic acid sequence at least about 80%, 85%, 90%, 91%, 92%, 93%, 94%, 95%, or 100% identical to at least about 15 contiguous nucleotides of a gene encoding a protein of TABLE 9C. In some embodiments, one or more gene modulatory reagents from the library of gene modulatory reagents comprise a nucleic acid sequence identical to at least about 15 contiguous nucleotides of a gene encoding a protein of TABLE 9C. In some embodiments, one or more gene modulatory reagents from the library of gene modulatory reagents comprise a nucleic acid sequence at least about 80%, 85%, 90%, 91%, 92%, 93%, 94%, 95%, or 100% identical to at least about 15 contiguous nucleotides of a gene encoding a protein of TABLE 9D. In some embodiments, one or more gene modulatory reagents from the library of gene modulatory reagents comprise a nucleic acid sequence identical to at least about 15 contiguous nucleotides of a gene encoding a protein of TABLE 9D. The identity may be least about 80% sequence identity. The identity may be least about 90% sequence identity.

In some cases, one or more of the gene modulatory reagents each comprise a gRNA sequence comprising at least about 90% identity to a sequence selected from SEQ ID NOS: 55-469. In some cases, one or more of the gene modulatory reagents each comprise a gRNA sequence comprising at least about 90% identity to a sequence selected from SEQ ID NOS: 460-1669. In some cases, one or more of the gene modulatory reagents each comprise a gRNA sequence comprising at least about 90% identity to a sequence selected from SEQ ID NOS: 1670-8119. In some cases, one or more of the gene modulatory reagents each comprise a gRNA sequence comprising at least about 90% identity to a sequence selected from SEQ ID NOS: 8120-12949. In some embodiments, each gene modulatory reagent comprises a gRNA sequence comprising homology to at least a portion of the gene that encodes a protein target of a therapeutic molecule in the library of therapeutic molecules. The gRNA may comprise homology to about 10 to about 50 contiguous nucleotides of the gene. The homology may be at least about 90% sequence identity. The gRNA may be positioned within a vector, e.g., for viral delivery as discussed herein.

The method of determining cancer cell susceptibility to the selected therapeutic molecule may further comprise contacting cells of a cell sample comprising cancer cells with an endonuclease. In some embodiments, the endonuclease comprises a Cas9 or Cas12a endonuclease. Non-limiting examples of Cas9 or Cas12a endonucleases include S. pyogenes Cas9 (SpCas9), SpCas9 D1135E variant, SpCas9 VRER variant, SpCas9 EQR variant, xCas9, SpCas9-NG, S. aureus Cas9 (SaCas9), Acidaminococcus sp. (AsCpf1), Lachnospiraceae bacterium (LbCpf1), AsCpf1 RR variant, LbCpf1 RR variant, AsCpf1 RVR variant, C. jejuni Cas9 (CjCas9), N. meningitidis (NmCas9), S. thermophilus (StCas9), T. denticola (TdCas9), and Mad7. In some embodiments, the endonuclease does not comprise a Cas9 or Cas12a endonuclease.

In some embodiments, the method of treating cancer in a subject may further comprise determining a cancer therapeutic for administering to the subject by uncovering a drug target dependency by combining a prediction of functional genomic gene dependency in a cell sample from the subject with results determined based on conventional genomic profiling for known oncogene or tumor suppressor mutations associated with the type of diagnosed cancer in the subject. In some embodiments, the prediction of functional genomic gene dependency in a cell sample from the subject is combined with positive identification of a mutation in a known oncogenic gene associated with the type of diagnosed cancer in the subject to select the cancer therapeutic for the administering.

In some embodiments, the gene modulatory reagents comprise a shRNA sequence comprising homology to at least a portion of the gene that encodes a protein target of a therapeutic molecule in the library of therapeutic molecules. The shRNA may comprise homology to about 10 to about 50 contiguous nucleotides of the gene. The homology may be at least about 90% sequence homology or identity. The shRNA may be positioned within a vector, e.g., for viral delivery as discussed herein.

As used in the specification and claims, the singular forms “a”, “an” and “the” include plural references unless the context clearly dictates otherwise. For example, the term “a sample” includes a plurality of samples, including mixtures thereof.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### EXAMPLES

The following examples are included for illustrative purposes only and are not intended to limit the scope of the invention.

### Example 1: Validating the CRISPR-Based Genetic Screen Platform Using a Previously Characterized Immortalized AML Cell Line

In this example, cell culture using the Kasumi-1 cell line (CRL-2724™ available from ATCC) was used to validate methods described herein using a CRISPR-based genetic screen to determine gene dependency for continued maintenance, growth, and/or proliferation of a myeloblast cell line. The Kasumi-1 cell line was isolated from a peripheral blood sample of an AML Asian male patient. The characterized cell type and morphology of this cells line are both myeloblast. The disease diagnosed in the host subject was acute myeloblastic leukemia.

### Known Genetic Characteristics of Kasumi-1 Cells

Kasumi-1 cells have a t (8:21) (q22:q22) karyotype. This translocation gives rise to an AML1-ETO fusion gene (also known as RUNX1-CBF2T1) which produces a chimeric AML1-ETO protein. The AML1-ETO chimeric oncoprotein prolongs survival of hematopoietic stem cells (HSCs) and inhibitions differentiation of HSCs. This chimeric oncoprotein has been shown to downregulate CEBPA activity which is essential for granulocyte differentiation. The full-length AML1-ETO fusion gene introduced in mouse models is not sufficient to induce leukemia in animals. This suggests that additional mutations or epigenetic interactions may be required for leukemogenesis within an t (8:21) (q22:q22) background that produces an AML1-ETO fusion protein. Activating mutations in the cytokine receptor tyrosine kinase KIT have been identified in approximately half of patients with t (8;21) AML indicating KIT functional interaction with an AML1-ETO fusion protein genetic background in AML development and/or disease progression. Kasumi-1 cells have been characterized to possess KIT gene dependency. Hence, Kasumi-1 cells serve as a model for clinical samples of AML cells with a known control gene (KIT) required for continued growth and AML cellular characteristics.

### Cell Culture

Kasumi-1 cells were maintained and proliferated in suspension. Cells were maintained in RPMI-1640 medium supplemented with 20% fetal bovine serum in a humidified incubator with 5% CO2 at 37° C.

Cells were maintained in sterile tissue culture containers at a density of between 3×10⁵ to 3×10⁶ viable cells/mL. Cell culture medium was replaced with fresh medium every 2-3 days. Kasumi-1 cells are positive for myeloperoxidase and demonstrate a morphology of myeloid maturation. Kasumi-1 cells are known to show a positive response to the cytokines IL-3, IL-6, G-CSF, and GM-CGF in cellular proliferation assays, but not to IL-1 or IL-5. Granulocytic maturation is not observed in Kasumi-1 cells upon addition of G-CSF or dimethyl sulfoxide. Eosinophilic maturation is not observed in Kasumi-1 cells upon addition of IL-5. Adding phorbol ester during culture of Kasumi-1 cells induces macrophage-like cells. Cell viability was determined by automated cell counting using Trypan blue exclusion or Acridine Orange/Propidium Iodide fluorescent method, which labeled dead and live with different dye colors. Live cell number was recorded. Then, cell concentration was adjusted to 1.2×10⁶/mL for an infection step with lentivirus containing a vector coding for CRISPRi gene modulation components described herein. The Lentivirus Infection Step and Infected cell culture step listed below in Example 2 were then used to assess operation of the CRISPRi gene modulation pathway in Kasumi-1 cells. The library of sgRNA sequences used in lentivirus in Example 1 target 120 genes and contained 350 different sgRNA elements selected from those listed in TABLE 9A and TABLE 9B. Gene dependency for these 120 target genes was assessed by next generation sequencing.

### Results

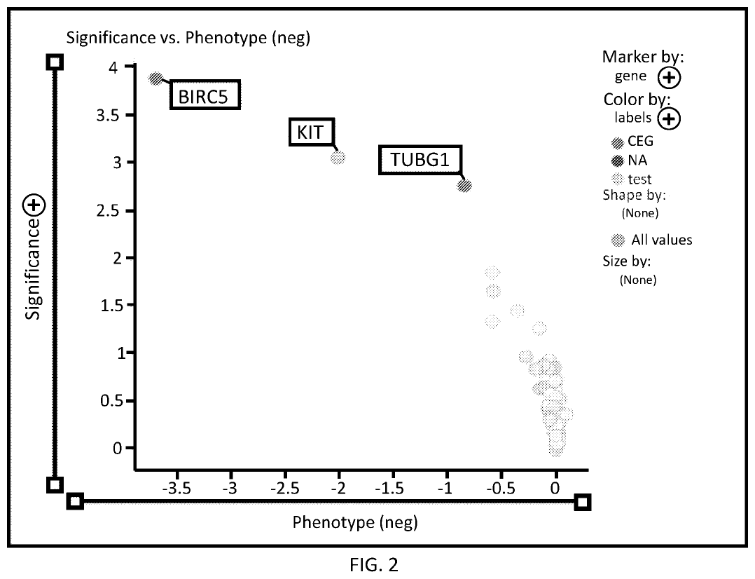

As shown in FIG. 2, scatter plots for gene dependency calling for CRISPRi-based gene modulation in Kasumi-1 cells indicated that knock-down of function for several genes was scored as significant indicating a dependency on function of those genes for Kasumi-1 maintenance, growth, and/or proliferation in cell culture after lentiviral infection. Among those genes scored as significant, were two known essential core genes (BIRC5 and TUBG1) and KIT. These result indicated that the CRISPRi gene modulation platform operates in an AML cell line to correctly identify positive control known essential genes (e.g., BIRC5 and TUBG1) and also to positively identify known AML-specific gene interactions (e.g., KIT) in this specific genetic background of Kasumi-1 cells. Hence, the CRISPRi gene modulation platform was validated as an AML functional gene dependency determination system.

### Example 2: Establishing Cell Cultures of Clinical Samples from Patients with Acute Myeloid Leukemia for CRISPR-Based Genetic Screen

In this example, cell cultures of clinical samples from acute myeloid leukemia (AML) patients were established for a CRISPR-based genetic screen.

### Cell Culture

Heme samples from peripheral blood draws or bone marrow biopsies from AML patients were sent directly to Function Oncology from affiliated medical centers. If fresh samples could be shipped immediately, blood samples were collected in vacutainers tubes and shipped directly to Function laboratory in a secured package containing cold packs. If samples cannot be sent fresh or only frozen samples were available, samples were processed at the medical center to remove red blood cells and freeze viable cells. Then, frozen samples were shipped in secured containers containing dry ice.

The median age of patients (for example from Fred Hutchison, Seattle, WA) was 54.2 years. The age range was 20 to 80 years old. If the information was available, the treatment regimens (e.g., chemotherapy and/or targeted therapies) and response assessment (e.g., remissions, relapse) were obtained from the collaborating teams.

Once samples were received, cells were recovered from frozen vials or freshly isolated blood samples. Frozen vials were thawed in water bath and then quickly spun down at 1,500 rpm for 10 minutes to remove preservative media. For the freshly available samples, red blood cells were removed using a Ficoll gradient density. The volume of blood or bone marrow biopsy was overlaid on top of a Ficoll-Hypaque media and tube spun at 2,000 rpm for 30 minutes. Then, the layer visible at the interface of red cell pellet and plasma was collected. Purified cells were washed with PBS and then counted. If not completed earlier by the clinical center, sample composition, e.g., blasts, myeloid and lymphocytes, and cell membrane markers profiling could be performed by flow cytometry.

Initial cultures were set up by seeding between 3-10 million cells in a non-treated culture vessel for a minimum of 24 hrs containing Stem Span (Stem Cell Technologies, Vancouver, BC) growth media supplemented with growth factors (SCF, TPO, IL-6, IL-3, FLT3L and GM-CSF) (Peprotech, Cranbury, USA)) at a final concentration of 10 ng/mL. The cells were maintained in a humidified incubator with 5% CO₂ at 37° C.

Cell viability was determined by automated cell counting using Trypan blue exclusion or Acridine Orange/Propidium Iodide fluorescent method, which labeled dead and live with different dye colors. Live cell number was recorded. Then, cell concentration was adjusted to 1.2×10⁶/mL for the infection step.

### Lentivirus Infection

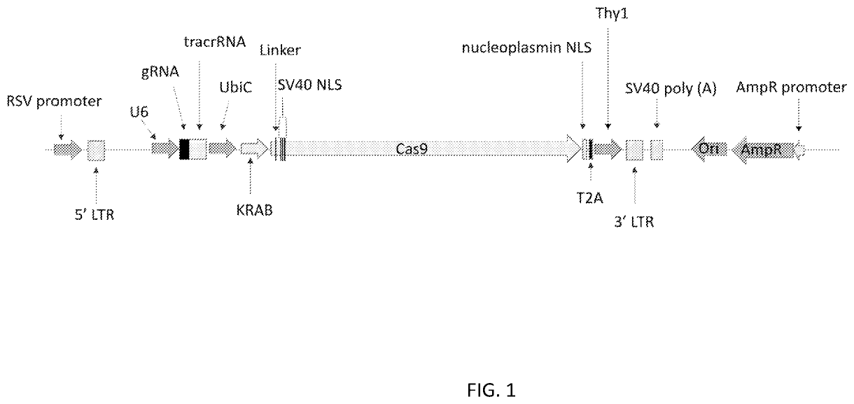

CRISPR-lentiviral particles required for infection and expression of the genes to achieve the screening procedure were generated independently. A 2^{nd} generation 3 vector system was used to generate lentivirus particles containing the CRISPR-Cas9 enzyme and sgRNA guide libraries listed in TABLES 9A-9D (sgRNAs targeting genes of interest and control sgRNAs). To produce fully functional particles, the library plasmid Cas9/sgRNA, as shown in FIG. 1, was co-transfected with the pVSV-G envelope plasmid and the p8.91 packaging construct (Gag-Pol, tat and rev). In order to detect virus integration and transgenes expression, murine Thy1 (mThy1) gene was also inserted into the lentivirus backbone. To enhance transfection, Lipofectamine was added to the transfection cocktail.

CRISPR-lentiviral particles were prepared using 293T cells. Briefly, 293T cells were expanded until confluency and transfected with envelop plasmid and packaging plasmid together with the CRISPR library plasmid and Lipofectamine. Transfection media (OptiMEM, Invitrogen, USA) was then replaced 6 hrs later and substituted with harvesting media (Advanced-DMEM, Invitrogen, USA). Every 12 hrs, harvesting media was collected and plates were replenished with fresh media. The virus was harvested at 12, 24, 36 and 48 hrs post-transfection.

Precipitation and concentration steps were performed using the PEG method. Briefly, virus was spun and harvested at 1500 rpm for 5 minutes to remove debris and filter the supernatant through 0.45 μM PES filter. Then the supernatant was mixed with PEG-8000 solution in 3:1 ratio and incubate with agitation at 4° C. for at least 4 hours. The solution was spun down at 4° C. and 4000 rpm for 1.5 hrs. The supernatant was removed, and the pellet was gently resuspended in 400 μL PBS and distributed in 20 μL aliquots. For long-term storage, the concentrated virus was kept at −80° C.

Virus stock concentration was assessed by transduction of target 293T cells. Cells were seeded at 10,000 cells in a 96 wells plate and serial dilutions of concentrated virus were added. 3 days later, the murine Thy 1 (mThy1) reporter gene co-expressed with the lenti-construct was detected by flow cytometry. Titer levels was calculated based on the Thy1 expression. The titer was between 5E7 and 10E7/mL Infectious Units per milliliter of concentrated virus media.

Transduction of the AML cells was performed in 96-well plate format. Approximately 200,000 cells were transduced with concentrated lentivirus at M.O.I of 1.2. Cell concentration in Stem Span media (Stem Cell Technologies, Vancouver, BC) with growth factors was adjusted to 1.2E6/mL. Lentivirus solution (250,000 IFUs in 30 μL) was mixed with 200,000 AML cells (170 μL of pre-made suspension). Spinfection was done by centrifugation for 20 minutes at 1,500 rpm. The cells were maintained in a humidified incubator with 5% CO₂ at 37° C.

### Infected Cells Culture

Following spinfection, cells were incubated with virus for 16 hrs. Cells were then transferred to a 12 well plate with fresh media to expand. Half of the cells from each well was harvested and pelleted by centrifugation at day 3. The remaining half was supplemented with fresh media for late time point. Media was changed at day 6. For final timepoint, day 8, cells were collected. Cells were stored for Next Generation Sequencing.

### Results

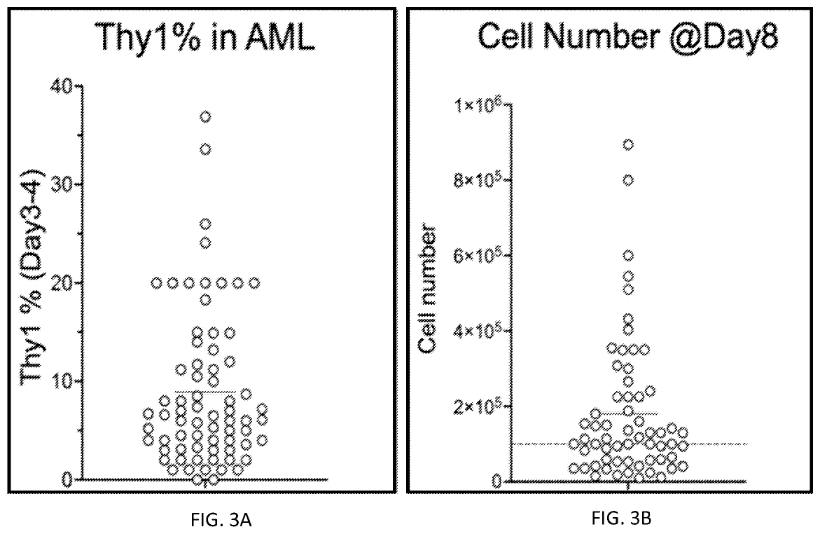

As shown in FIGS. 3A-3B, transduction efficiency and cell numbers were quantified, and multiple samples from AML patients were infected with CRISPR-Library lentivirus. Cell surface expression of mThy1 was quantified by flow cytometry to investigate transduction efficiency in the cell population. The data in FIG. 3A showed mThy1 quantification in 63 individual AML samples on 3 days post lentivirus infection. Expression of mThy 1 by flow cytometry was detected using APC-conjugated antibody and samples run on a Novocyte cytometer. Transduction above 5% was considered as successful. On average, a 10% transduction efficiency was equivalent to a 100× library representation. Samples transduced at 5% or more were subsequently designated for NGS analysis. Control samples without virus were used as negative control for mThy1 expression.

In parallel, to assess in vitro cell behavior and survival or proliferation, cell numbers were quantified at day 8. FIG. 3B showed the cell counting of 63 individual AML samples collected at the end of the CRISPR-screen experiment. The dotted line indicates the starting number of cells. Cells were harvested at Day 8 and individually counted using hemocytometer and trypan blue exclusion dye.

Overall, these results show a successful establishment of CRISPR-based genetic screen using cells from AML patients.

### Example 3: Identification of Target Genes Using CRISPR-Based Genetic Screen from AML Cells

In this example, data from the CRISPR-based genetic screen generated from the previous example using AML cells was obtained and analyzed.

### Next-Generation Sequencing

Genomic DNA was extracted using Zymo DNA kit protocol and gDNA was stored for NGS analysis. The pool of sgRNA amplicons was amplified and multiplexed using PCR method using forward primers with unique barcodes and a common reverse primer. The whole amount of gDNA, which could be split in multiple 1 μg DNA reaction if needed, was amplified using Q5-NEB High-Fidelity 2×PCR Master Mix.

The following PCR setting was used: 98° C./3 min; 25 cycles of 98° C./10s, 60° C./15s, 72° C./60s, and 72° C./2 min.

All PCR amplifications for each sample were examined by gel electrophoresis in 2% agarose and the amplicons' quality was determined with an expected amplicon size of about 350 bp. Then, amplicons were purified using Zymo magnetic beads extraction kit.

A Qubit fluorometer (Thermo Fisher Scientific) was used to quantify samples labeled with different indices. All samples were then pooled in equimolar amounts and were deep sequenced using Illumina MiSeq 4000 platform at Admera Health. Sequencing runs were analyzed using algorithm specifically designed for CRISPR screen experiments.

### Results

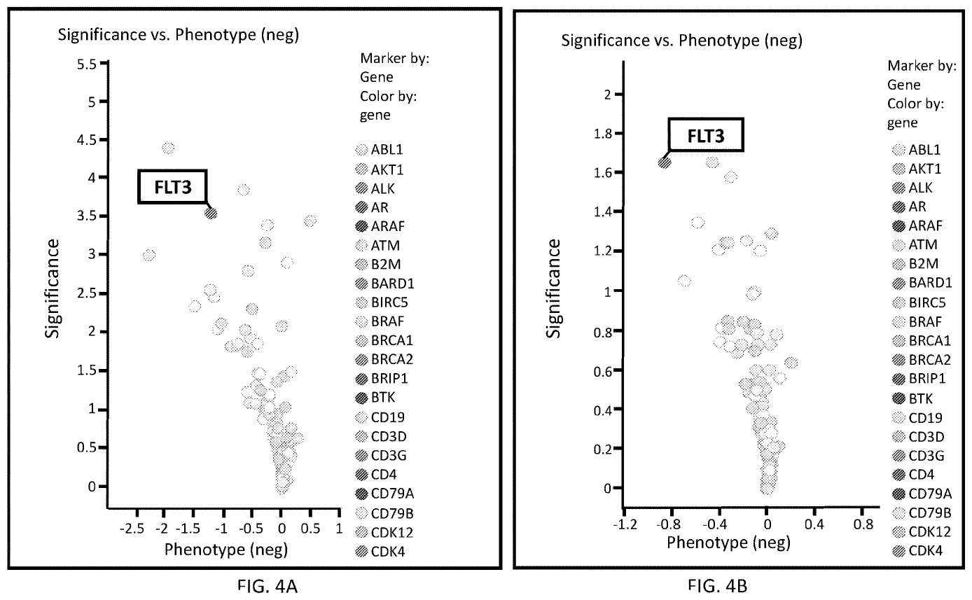

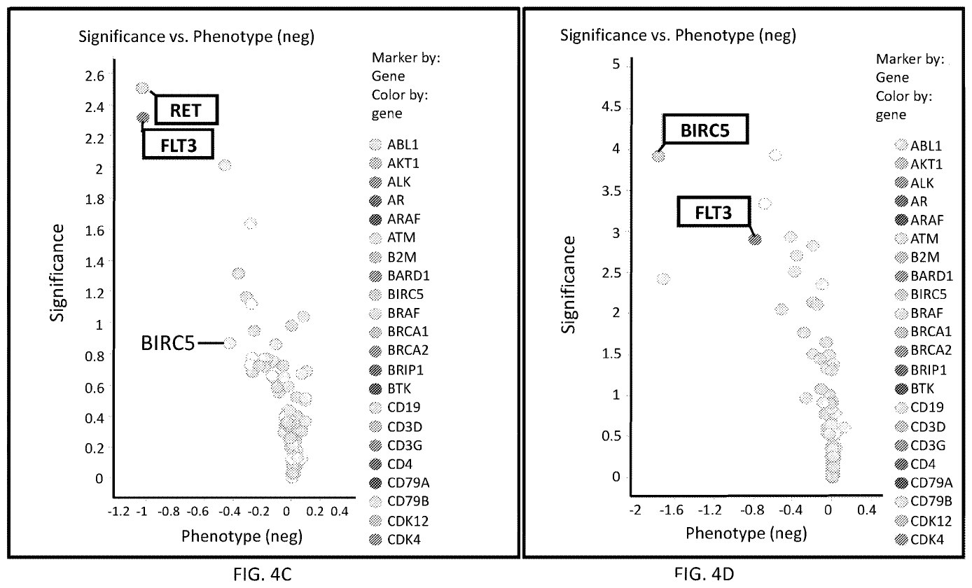

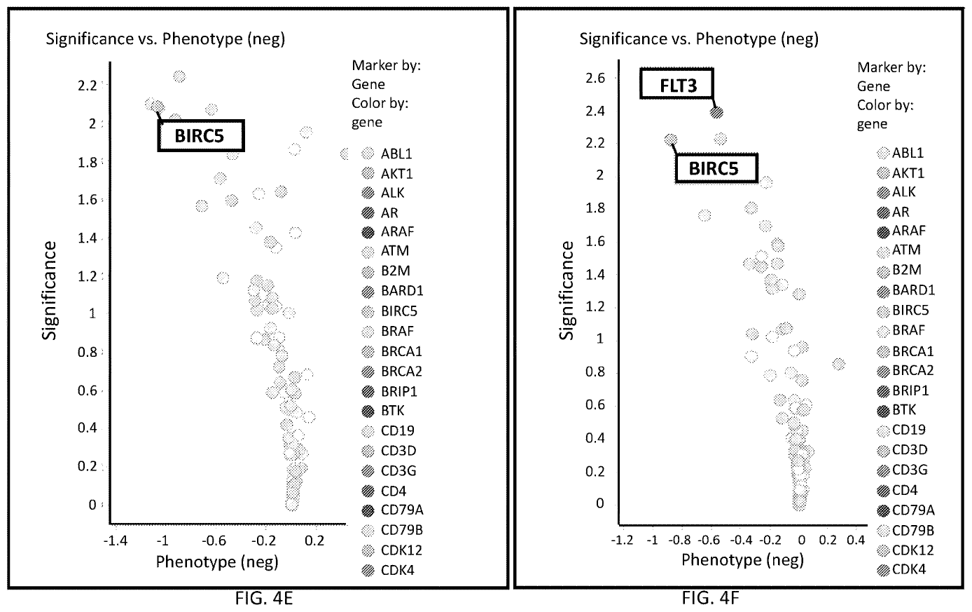

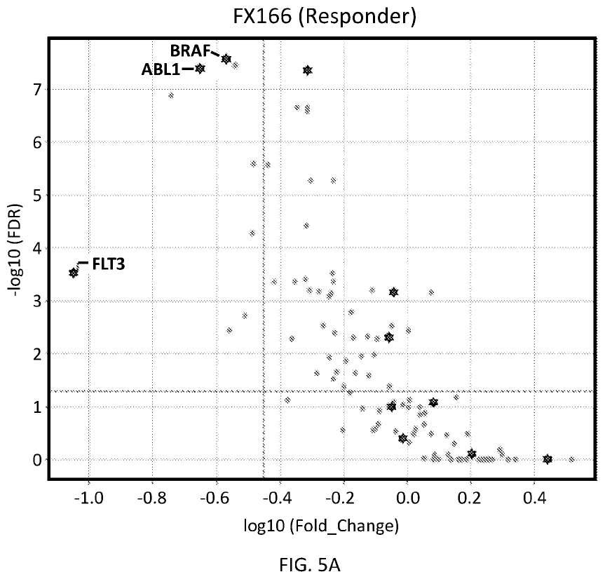

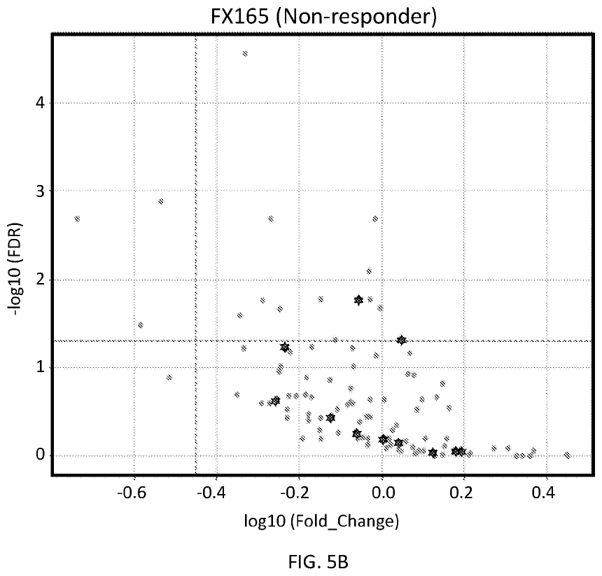

FIG. 4A-FIG. 4F show scatter plots of 6 representative CRISPR screens on primary AML samples. In this experiment, genomic DNA was extracted, and specific primers were used to amplify the CRISPR-library amplicon by PCR. Then, sample barcoding was generated using Illumina primers following supplier's recommendations and amplicons were sequenced. Data processing was performed in house, including read counts, enrichment or depletion of guides calculation. The CB2 statistical package was used for data analysis to compare read counts from Day 3 and Day 8. FIG. 4A-FIG. 4F depict typical data representations of gene dependencies in 6 AML samples, with cut-off FDR<0.1 and Log FC<−0.3. The results show that CRISPR-based screen allows identification of FLT3, RET (b-Raf pathway) and BIRC5 as essential genes for AML survival.

### CONCLUSIONS

These genes can be targeted for therapeutic development or treatment. These genes can be targeted in a method of treatment comprising administering a selected targeted cancer therapeutic to the subject wherein the selected targeted cancer therapeutic targets the determined functional gene dependency in the cancer cells from the subject.

### Example 4: Studies Operating a CRISPR-Based Genetic Screening Platform on Pre-Therapy Samples from Selected Cohorts of AML Patients

In this example, data from a CRISPR-based genetic screen using AML cells taken from a selected cohort of subjects prior to initiation of a therapy was obtained and analyzed.

### Cell Sample Collection, Maintenance, and CRISPRi Genetic Modification by Lentiviral Transduction

Fresh or viably frozen samples of peripheral blood or bone marrow from AML patients were obtained and shipped overnight to the laboratory for CRISPR-based genetic screening. The samples were checked for “blast” cell (cancerous cells) markers which indicated the proportion of cancer cells in the whole sample. In general, CD34 and CD33 are the common markers used for the “blast” subset. This procedure was exclusively performed by the clinical lab prior to freezing and shipping the samples. Cells were recovered from frozen vials or freshly isolated blood samples and red blood cells, if present, were removed using Red Cell Lysis buffer. No AML cell enrichment procedures (e.g., enrichment for blast subset or removal of non-cancer cells) were performed prior to sample collection and freezing or following sample thawing and maintenance in cell culture. Remaining cells from the samples, comprising white blood cells, were cultured in standard growth factor (TPO, SCF, FLT3L, IL6, IL3, GM-CSF) supplemented media (Stem Cell Technologies) and maintained in a humidified incubator with 5% CO₂ at 37° C. Cell viability was determined by automated cell counting (Acridine Orange/Propidium Iodide method) and cell concentration of the cultured cells was adjusted on this basis prior to lentivirus infection. Sorafenib-treated samples were obtained post therapy but were collected from subjects prior to the initiation of sorafenib treatment. Clinical information provided prior to and during analysis was limited to type and method of cancer therapeutic treatment, clinical response of the subject, and eventually any prior treatment when available.

Lentivirus was used to deliver CRISPR-Cas9 and sgRNA components into the target cells. The CRISPR-Cas9 and sgRNA components were encoded in single vectors comprising nucleotide coding sequence for: an RSV promoter; a 5′ long terminal repeat (LTR) under transcriptional regulation of the RSV promoter; a U6 promoter; a single guide RNA (sgRNA) comprising a guide RNA (gRNA) sequence listed in TABLE 9A or TABLE 9B selected from a sequence from SEQ ID NOs: 55-1669 and a tracrRNA sequence under transcriptional regulation of the U6 promoter; a UbiC promoter; a single polypeptide sequence comprising a sequence containing the KRAB domain region derived from a human KRAB domain protein, a flexible linker sequence, an SV40 NLS, a dCas9 sequence, a nucleoplasmin NLS, a T2A polypeptide sequence, and a Thy1 sequence under transcriptional regulation of the UbiC promoter. Concentrated lentivirus (in-house virus production) containing all the elements of the gRNA library pool was prepared by standard procedures in 293T cells. Virus titer was determined by flow-cytometry using an encoded marker gene. Cell samples comprising AML cells were infected by spinfection, in replicate at MOI~1. Following infection, cells were maintained in a humidified incubator with 5% CO₂ at 37° C.

Following transduction, cells derived from the subject comprising CRISPRi genetically-modified cells, were cultured for 72 hours, at which time a baseline sample (input sample) was harvested and frozen at −20° C. The remaining cells were supplemented with fresh media, cultured for 5 additional days, and then frozen at −20° C. (final sample).

Cell pellets were then collected from the freezer and genomic DNA (gDNA) was extracted from each of two replicates for input and final samples. gDNA concentration and yield were measured and an aliquot was used to assess viral copy number (VCN) for Quality Control.

### Molecular Biology: Library Preparation and NGS Protocol

The remaining gDNA was used to prepare PCR amplicons containing the gRNA sequences of all integrated lentivirus vectors (LVV). This is a 2-stage protocol with a limited number of cycles performed in the first round, followed by a second round which added sample index barcodes and flow-cell binding sequences. An aliquot was examined by gel electrophoresis for Quality Control purposes. Amplicons were purified and quantitated using standard methods, and then pooled in equimolar ratio for next generation DNA sequencing (NGS). NGS was performed using the MiSeq instrument (Illumina), operated by Admera Health, or the MiniSeq instrument (Illumina) at an on-site laboratory, per manufacturer instructions.

### Mutational Analysis of Clinical Samples

Mutation data for each sample was obtained directly from the clinical site. Following sample collection, an NGS panel for several genes known to be associated with AML was used to assay an aliquot of each sample. In parallel, some samples are also assayed at an in-house laboratory using Whole Exon Sequencing to identify all mutations residing within exons associated with the clinical samples.

### Retrospective Study on Pre-Therapy AML Samples

To demonstrate clinical relevance of CRISPR-based functional genomics for AML treatment, CRISPRi-based methods of genetic modulation described herein were applied to viably banked tumor cell samples from patients diagnosed with acute myeloid leukemia (AML). Pre-treatment peripheral blood mononuclear cells were obtained from 22 patients with newly diagnosed AML, all of whom were subsequently treated with a combination of the multi-kinase inhibitor sorafenib and chemotherapy (mostly commonly the GCLAM regimen). Sorafenib is a molecularly targeted agent which potently inhibits multiple serine/threonine and tyrosine kinases. Importantly, many physiologically relevant targets of sorafenib are known beyond FLT3, including RAF paralogues (BRAF, RAF1, ARAF), PDGFRα/β.

This assay was performed on banked PBMCs as described above in Example 3. Complete response to treatment without molecular residual disease (CR without MRD) was defined as ‘expected’ whenever Gene Dependency (GD) was observed for any primary sorafenib target (i.e., FLT3, FLT4, ABL1, BRAF, RAF1, ARAF, KDR, KIT, PDGFRA/B, RET), regardless of mutational status. Treatment response was assessed using standard criteria (bone marrow measurement of blast percentage), with additional assessment for molecular residual disease by either flow cytometry or NGS. Importantly, the backbone chemotherapy regimen administered in this cohort has been reported to frequently yield complete responses in newly-diagnosed AML.

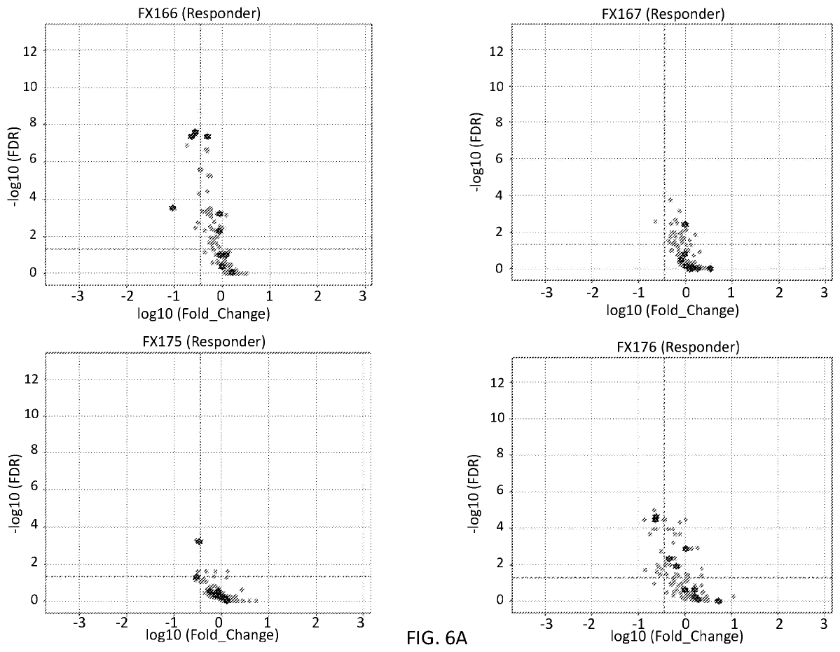

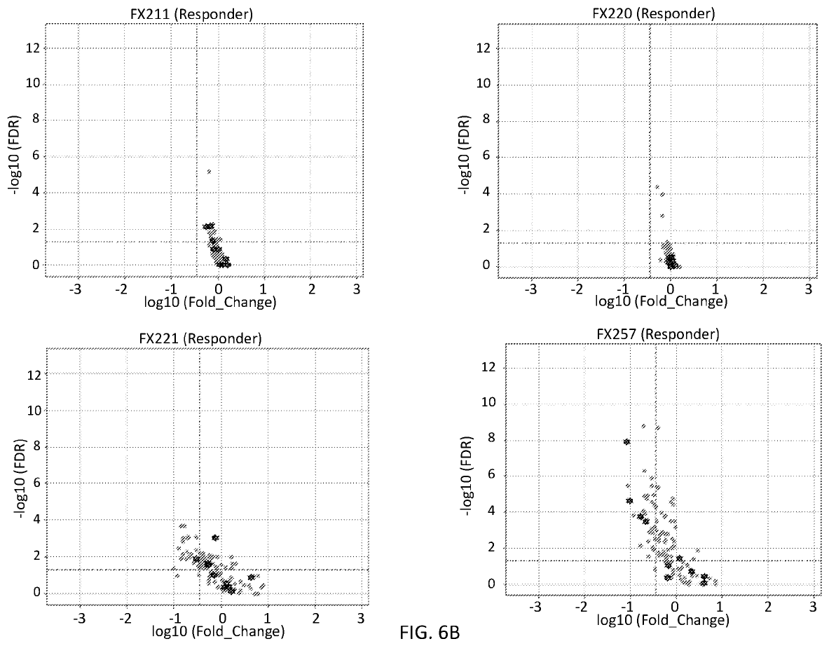

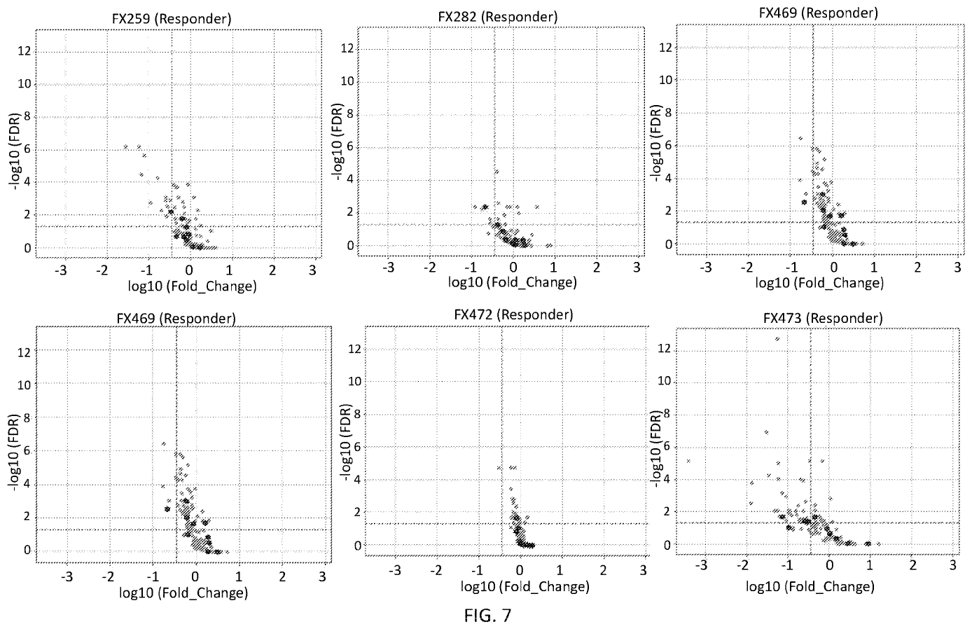

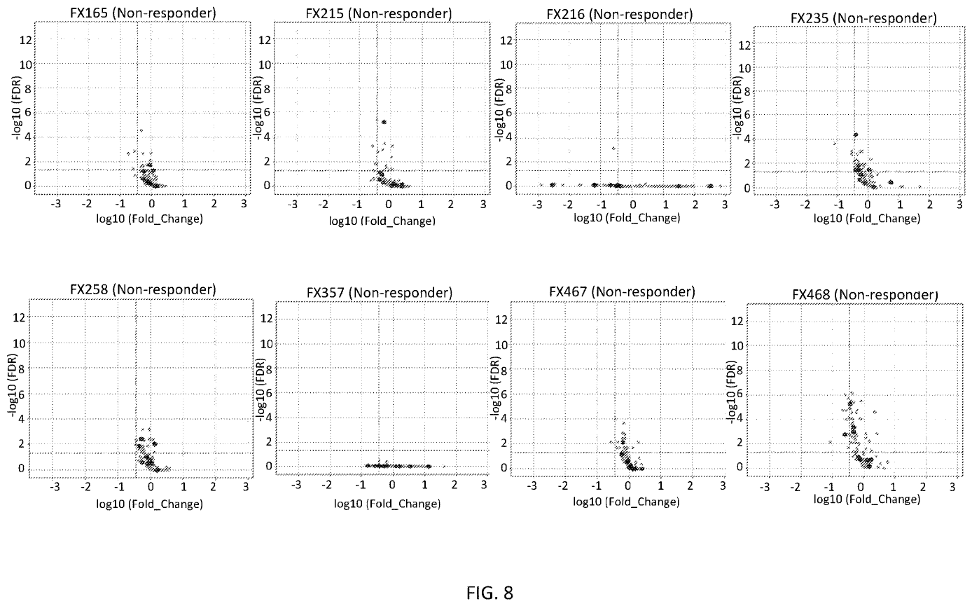

Among the 22 AML patients included in this analysis, 14 reached CR and 8 were classified as non-responders with refractory or molecular residual disease (MRD). Patients were predicted to respond if they demonstrated functional dependence on at least one primary sorafenib target. Compared with stratification using routine genomics (e.g., FLT3 mutation), GD predicted response to sorafenib appeared more consistent with observed clinical response. As an example, patient FX166, who received sorafenib+GCLAM treatment and achieved complete response, tested negative for mutations in genes typically regarded as genomic biomarkers for AML therapy. Patient FX166 tested FLT3 wild-type (WT), IDH1 WT, and IDH2 WT). The results of running this CRISPRi-based genome modulation assay indicated dependency on multiple sorafenib targets, thus predicting positive sorafenib response, which was consistent with the observed clinical response. Conversely, patient FX165 also received sorafenib+GCLAM treatment, but had detectable MRD. Despite the presence of the canonical ITD mutation in FLT3, the CRISPRi-based genome modulation assay did not detect dependency on sorafenib targets. The GD predicted response for this patient (non-responder) is thus consistent with the observed clinical response. FIG. 5A shows scatter plot results to support the conclusion of FLT3 tumor cell dependency and sorafenib+GCLAM responsiveness in Patient FX166 despite testing FLT3 wild-type (WT), IDH1 WT, and IDH2 WT for genomic analysis. FIG. 5B shows scatter plots following operation of the CRISPRi-based method of functional genomic modulation to support the conclusion of no significant FLT3 tumor cell dependency in Patient FX165 which predicted that the patient would be a non-responder to sorafenib+GCLAM treatment despite the presence of the canonical ITD mutation in FLT3. FIG. 5A-FIG. 5B demonstrate calling of functional gene dependency following operation of the CRISPRi-based method of functional genomic modulation for two samples from AML patients in which the functional gene dependency matched the clinical outcome following treatment but was not predicted based on the results of genomic analysis of known AML causing mutations. Each panel in FIG. 5A-FIG. 5B shows the scatterplot for one patient sample, with sample ID and observed response status labeled in panel heading. X-axis is the logarithm of read count fold change while the y-axis is the logarithm of false discover rate (FDR). The dashed lines are cutoffs (FDR<=0.05 and log fold change <=−0.45) used for gene dependency calling. Data points outlined in stars represent primary sorafenib target genes (e.g., FLT3, FLT4, ABL1, ARAF, BRAF, RAF1, KDR, KIT, RET, PDGFRA/B) and data points in gray dots are the remainder of the library panel genes tested with gRNA sequences selected from TABLES 9A and 9B. AML cells from patient FX166 (who was established to be a sorafenib responder following therapeutic treatment) were determined to be functionally dependent on the known sorafenib target genes FLT3, ABL1, and BRAF through CRISPRi screening. In this instance, a positive clinical response to sorafenib was predicted for a patient that matched with the determined clinical outcome. AML cells from patient FX165 (who was established to be a sorafenib non-responder following therapeutic treatment) had no known sorafenib target genes identified as being functionally dependent through CRISPRi screening indicating the functional prediction for response to sorafenib matched with the determined clinical outcome. FIG. 6A, FIG. 6B and FIG. 7 show results for CRISPRi screens on AML cells from 14 patients determined to be clinically responsive to sorafenib treatment. FIG. 8 show results for CRISPRi screens on AML cells from 8 patients determined to be clinically non-responsive to sorafenib treatment. Each panel in FIG. 6A, FIG. 6B, FIG. 7, and FIG. 8 shows the scatterplot for one patient sample, with sample ID and observed response status labeled in panel heading. X-axis is the logarithm of read count fold change while the y-axis is the logarithm of FDR. The dashed lines are cutoffs (FDR<=0.05 and log fold change <=−0.45) used for gene dependency calling. Data points plotted in a star outline represent primary sorafenib target genes (e.g., FLT3, FLT4, ABL1, ARAF, BRAF, RAF1, KDR, KIT, RET, PDGFRA/B) and data points in gray dots are the remainder of the assayed genes.

Comparing the GD predicted response against observed response across the cohort of 22 AML patients, the prediction performance was 78.6% sensitivity, 87.5% specificity, 91.7% PPV and 70.0% NPV (TABLE 12-confusion matrix table for sorafenib response prediction; FIG. 6A, FIG. 6B, FIG. 7, and FIG. 8—scatter plots for gene dependency calling for all 22 patient samples). Similar comparisons were conducted for available mutation data of primary sorafenib target genes (FLT3 and KIT mutations) against observed response and showed inferior association between mutation and response than GD. The prediction performance for FLT3 mutations only is 64.3% sensitivity, 50% specificity, 69.2% PPV and 44.4% NPV, while for FLT3 and KIT mutations combined is 71.4% sensitivity, 37.5% specificity, 66.7% PPV and 42.9% NPV.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Aspects

Aspect 1: A CRISPR interference (CRISPRi) system comprising a single vector transfer plasmid system comprising:
a) a first promoter operably linked to a first nucleotide sequence encoding a catalytically inactive RNA-directed nuclease fused to a transcriptional repressor; and
b) a second promoter operably linked to a second nucleotide sequence encoding a single-guide RNA (sgRNA) sequence comprising a CRISPR RNA (crRNA) sequence having complementarity to at least a portion of a gene that encodes a protein target and a trans-activating CRISPR RNA (tracrRNA) sequence.
Aspect 2: The CRISPRi system of aspect 1, wherein the second nucleotide sequence encodes an sgRNA sequence comprising a nucleic acid sequence corresponding to at least 5 contiguous bases and up to at most 30 contiguous bases from the sense strand or the antisense strand of a gene sequence selected from SEQ ID NOs: 55-12949.
Aspect 3: The CRISPRi system of aspect 1 or 2, wherein the second nucleotide sequence encodes an sgRNA sequence comprising a nucleic acid sequence corresponding to the sense strand or the antisense strand of a gene sequence selected from SEQ ID NOs: 55-12949.
Aspect 4: The CRISPRi system of any one of aspects 1-3, wherein the second nucleotide sequence encodes an sgRNA sequence comprising a nucleic acid sequence corresponding to the sense strand or the antisense strand of a gene sequence selected from SEQ ID NOs: 55-1669.
Aspect 5: The CRISPRi system of any one of aspects 1-3, wherein the second nucleotide sequence encodes an sgRNA sequence comprising a nucleic acid sequence corresponding to the sense strand or the antisense strand of a gene sequence selected from SEQ ID NOs: 1670-8119.
Aspect 6: The CRISPRi system of any one of aspects 1-3, wherein the second nucleotide sequence encodes an sgRNA sequence comprising a nucleic acid sequence corresponding to the sense strand or the antisense strand of a gene sequence selected from SEQ ID NOs: 8120-12949.
Aspect 7: The CRISPRi system of any one of aspects 1-6, wherein the single vector transfer plasmid system comprises a single vector transfer plasmid formatted for co-transfection with one or more viral packaging plasmids.
Aspect 8: The CRISPRi system of any one of aspect 1-7, comprising one or more viral packaging plasmids.
Aspect 9: The CRISPRi system of aspect 8, wherein the one or more viral packaging plasmids comprise nucleotide coding sequence to encode lentiviral particles, retroviral particles, adenoviral particles, adeno-associated viral particles, or herpes simplex viral particles.
Aspect 10: The CRISPRi system of aspect 9, wherein the one or more viral packaging plasmids comprise nucleotide coding sequence to encode lentiviral particles.
Aspect 11: The CRISPRi system of any one of aspects 1-10, wherein a) and b) are contained within the single vector transfer plasmid.
Aspect 12: The CRISPRi system of any one of aspects 1-11, wherein the single vector transfer plasmid has a plasmid size of less than about 9.2 kilobases (kb), 9 kb, 8.7 kb, 8.5 kb, 8 kb, 7.5 kb, 7 kb, 6.5 kb, 6 kb, 5.5 kb, 5 kb, 4.5 kb, 4 kb, 3.5 kb, 3 kb, 2.5 kb, or 2 kb.
Aspect 13: The CRISPRi system of any one of aspects 1-12, wherein the single vector transfer plasmid has a plasmid size of at least than about 8.6 kb, 8.5 kb, 8 kb, 7.5 kb, 7 kb, 6.5 kb, 6 kb, 5.5 kb, 5 kb, 4.5 kb, 4 kb, 3.5 kb, 3 kb, 2.5 kb, or 2 kb.
Aspect 14: The CRISPRi system of any one of aspects 1-13, wherein the catalytically inactive RNA-directed nuclease is dCas9, dCas12a, dCas12f1, dCasMINI, or a variant thereof.
Aspect 15: The CRISPRi system of any one of aspects 1-14, wherein the first nucleotide sequence comprises coding sequence for a dCas9 polypeptide, a dCas12a polypeptide, a dCas12f1 polypeptide, or a dCasMINI polypeptide, or a variant thereof.
Aspect 16: The CRISPRi system of aspect 15, wherein the coding sequence for the dCas9 polypeptide encodes a dCas9 polypeptide at least about 90% identical to the amino acid sequence of SEQ ID NO: 12950.
Aspect 17: The CRISPRi system of aspect 15 or 16, wherein the coding sequence for the dCas9 polypeptide encodes a dCas9 polypeptide comprising the amino acid sequence of SEQ ID NO: 12950.
Aspect 18: The CRISPRi system of aspect 15, wherein the coding sequence for the dCas 12a polypeptide encodes a dCas 12a polypeptide at least about 90% identical to the amino acid sequence of SEQ ID NO: 12951.
Aspect 19: The CRISPRi system of aspect 15 or 18, wherein the coding sequence for the dCas 12a polypeptide encodes a dCas 12a polypeptide comprising the amino acid sequence of SEQ ID NO: 12951.
Aspect 20: The CRISPRi system of aspect 15, wherein the coding sequence for the dCas12f1 polypeptide encodes a dCas12f1 polypeptide at least about 90% identical to the amino acid sequence of SEQ ID NO: 12952.
Aspect 21: The CRISPRi system of aspect 15 or 20, wherein the coding sequence for the dCas12f1 polypeptide encodes a dCas12f1 polypeptide comprising the amino acid sequence of SEQ ID NO: 12952.
Aspect 22: The CRISPRi system of aspect 15, wherein the coding sequence for the dCasMINI polypeptide encodes a dCasMINI polypeptide at least about 90% identical to the amino acid sequence of SEQ ID NO: 12953.
Aspect 23: The CRISPRi system of aspect 15 or 22, wherein the coding sequence for the dCasMINI polypeptide encodes a dCasMINI polypeptide comprising the amino acid sequence of SEQ ID NO: 12953.
Aspect 24: The CRISPRi system of any one of aspects 1-23, wherein the transcriptional repressor comprises a sequence of KRAB (also referred to as KOX), SID, MBD2, MBD3, HP1a, DNMT family (including DNMT1, DNMT3A, DNMT3B, DNMT3L, DNMT2A), Sin3a, Rb, MeCP2, ROM2, AtHD2A, LSD1, SUV39H1, G9a (EHMT2), ZFP1, ZFP14, ZFP82, ZIM2, ZIM3, ZNF18, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF419, ZNF140, ZNF175, ZNF214, ZNF184, ZNF8, ZNF60, ZNF595, ZNF596, ZNF10, ZNF37A, ZNF394, ZNF610, ZNF273, ZNF34, ZNF250, ZNF98, ZNF675, ZNF213, ZFP28-2, ZNF224, ZNF257, GLP (EHMT2), or a variant or combination thereof.
Aspect 25: The CRISPRi system of aspect 24, comprising the KRAB, wherein the KRAB comprises a KRAB protein domain, or variant thereof, derived from one or more of proteins listed in Table 1 or Table 2.
Aspect 26: The CRISPRi system of claim 25, wherein the KRAB protein domain, or variant thereof, comprises a sequence at least about 90%, 91%, 92%, 93%, 94%, 95%, 96%, 97%, or 98% identical to a portion of at least 60 contiguous amino acids of a sequence selected from SEQ ID NOs: 1-54.
Aspect 27: The CRISPRi system of any one of aspects 24-26, wherein the transcriptional repressor comprises a KRAB protein domain derived from ZFP1, ZFP14, ZFP82, ZIM2, ZIM3, ZNF140, ZNF175, ZNF18, ZNF250, ZNF37A, ZNF419, ZNF595, ZNF60, or ZNF8.
Aspect 28: The CRISPRi system of any one of aspects 24-27, wherein the transcriptional repressor comprises a KRAB protein domain comprising an amino acid sequence corresponding to the KRAB domain coordinates listed in Table 1 or Table 2.
Aspect 29: The CRISPRi system of any one of aspects 1-28, wherein the catalytically inactive RNA-directed nuclease fused to the transcriptional repressor is contained within a single polypeptide chain and the catalytically inactive RNA-directed nuclease is separated from the transcriptional repressor by a peptide linker comprising about 1 to about 100 amino acids.
Aspect 30: The CRISPRi system of aspect 29, wherein the peptide linker comprises a flexible linker (e.g., about 16 amino acids).
Aspect 31: The CRISPRi system of aspects 29 or 30, wherein the peptide linker comprises SEQ ID NO: 12954.
Aspect 32: The CRISPRi system of any one of aspects 1-31, wherein the catalytically inactive RNA-directed nuclease comprises a nuclear localization sequence (NLS) positioned amino-terminal (N-terminal) to a RuvC-1 domain of the CAS endonuclease.
Aspect 33: The CRISPRi system of aspect 32, wherein the NLS positioned N-terminal to the RuvC-1 domain comprises a triple SV40 NLS.
Aspect 34: The CRISPRi system of any one of aspects 1-33, wherein the catalytically inactive RNA-directed nuclease comprises an NLS positioned carboxy-terminal (C-terminal) to a RuvC-III domain of the CAS endonuclease.
Aspect 35: The CRISPRi system of claim 238, wherein the NLS positioned C-terminal to the RuvC-III domain comprises a single nucleoplasmin NLS.
Aspect 36: The CRISPRi system of any one of aspects 1-35, wherein the catalytically inactive RNA-directed nuclease comprises a peptide-linked downstream reporter C-terminal to a RuvC-III domain of the catalytically inactive RNA-directed nuclease.
Aspect 37: The CRISPRi system of aspect 36, wherein the peptide-linked downstream reporter C-terminal to the RuvC-III domain comprises a self-cleaving T2A-peptide linked downstream reporter.
Aspect 38: The CRISPRi system of aspect 37, wherein the self-cleaving T2A-peptide linked downstream reporter is linked to the catalytically inactive RNA-directed nuclease C-terminal relative to the NLS positioned C-terminal to the RuvC-III domain.
Aspect 39: The CRISPRi system of aspect 37 or 38, wherein the self-cleaving T2A-peptide linked downstream reporter comprises Thy1.
Aspect 40: The CRISPRi system of any one of aspects 1-39, wherein the single vector encodes a first promoter sequence, wherein the first promoter sequence is operably-linked to coding sequence of the catalytically inactive RNA-directed nuclease.
Aspect 41: The CRISPRi system of aspect 40, wherein the first promoter sequence comprises a UbiC promoter sequence.
Aspect 42: The CRISPRi system of any one of aspects 1-41, wherein the single vector encodes a second promoter sequence, wherein the second promoter sequence is operably-linked to coding sequence of the sgRNA.
Aspect 43: The CRISPRi system of aspect 42, wherein the second promoter sequence comprises a U6 promoter sequence.
Aspect 44: The CRISPRi system of any one of aspects 1-43, wherein the single vector encodes a 5′ LTR sequence and a 3′ LTR sequence.
Aspect 45: The CRISPRi system of aspect 44, wherein the 5′ LTR sequence and/or the 3′ LTR sequence comprises a deletion that creates a self-inactivating vector.
Aspect 46: A composition comprising a high titer CRISPRi lentiviral gene modulation solution comprising the CRISPRi system of any one of aspects 1-45.
Aspect 47: The composition of aspect 46, wherein the high titer CRISPRi lentiviral gene modulation solution is sufficient to transduce a majority of primary cells maintained in culture, wherein the primary cells are derived from a subject.
Aspect 48: The composition of aspect 46 or 47, wherein the high titer CRISPRi lentiviral gene modulation solution is sufficient to transduce at least about 50%, 55%, 60%, 65%, 70%, 75%, 80%, 85%, 90%, 95%, 98%, or 99% of primary cells maintained in culture, wherein the primary cells are derived from a subject.
Aspect 49: The composition of any one of aspects 46-48, wherein the high titer CRISPRi lentiviral gene modulation solution is concentrated to a titer of at least 1×10⁷, 2×10⁷, 3×10⁷, 4×10⁷, 5×10⁷, 6×10⁷, 7×10⁷, 8×10⁷, 9×10⁷, or 1×10⁸ Infectious Units/mL.
Aspect 50: The composition of any one of aspects 46-49, wherein the high titer CRISPRi lentiviral gene modulation solution is concentrated to a titer of at most about 1×10⁸ or 5×10⁷ Infectious Units/mL.
Aspect 51: The composition of any one of aspects 46-50, wherein the high titer CRISPRi lentiviral gene modulation solution is concentrated to a titer between about 1×10⁷-1×10⁸ Infectious Units/mL.

### Tables

TABLE 1
Exemplary human KRAB domain proteins

| | | | | KRAB domain |
|---|---|---|---|---|
| | | | | coordinates |
| Gene Symbol | Description | Uniprot ID | Gene ID | (aa numbering) |
| KRBA1 | KRAB-A domain containing 1 | A0A0C4DH65 | 84626 | 6 . . . 85 |
| KRBA2 | KRAB-A domain containing 2 | A0A2R8Y7G8 | 124751 | 8 . . . 41 |
| KRBOX1 | KRAB box domain containing 1 | A0A024R2N1 | 100506243 | 14 . . . 86 |
| KRBOX4 | KRAB box domain containing 4 | Q5JUW0 | 55634 | 7 . . . 79 |
| KRBOX5 | KRAB box domain containing 5 | Q7Z2F6 | 124411 | 4 . . . 75 |
| POGK | pogo transposable element derived | A0A024R8Y1 | 57645 | 47 . . . 118 |
| | with KRAB domain | | | |
| PRDM7 | PR/SET domain 7 | Q9NQW5 | 11105 | 26 . . . 96 |
| PRDM9 | PR/SET domain 9 | Q9NQV7 | 56979 | 26 . . . 96 |
| RBAK | RB associated KRAB zinc finger | Q9NYW8 | 57786 | 7 . . . 79 |
| RBAK-RBAKDN | RBAK-RBAKDN readthrough | A0A0A6YYG8 | 100533952 | 7 . . . 79 |
| SSX1 | SSX family member 1 | Q16384 | 6756 | 23 . . . 82 |
| SSX2 | SSX family member 2 | Q16385 | 6757 | 23 . . . 82 |
| SSX3 | SSX family member 3 | A0A024R1B1 | 10214 | 23 . . . 82 |
| SSX4 | SSX family member 4 | Q60224 | 6759 | 23 . . . 82 |
| SSX5 | SSX family member 5 | Q60225 | 6758 | 23 . . . 82 |
| SSX7 | SSX family member 7 | A0A024R019 | 280658 | 23 . . . 82 |
| ZFP1 | ZFP1 zinc finger protein | H3BV40 | 162239 | 7 . . . 76 |
| ZFP14 | ZFP14 zinc finger protein | Q9HCL3 | 57677 | 5 . . . 78 |
| ZFP28 | ZFP28 zinc finger protein | Q8NHY6 | 140612 | 102 . . . 174, |
| | | | | 231 . . . 299 |
| ZFP30 | ZFP30 zinc finger protein | D3Y2A0 | 22835 | 5 . . . 78 |
| ZFP37 | ZFP37 zinc finger protein | Q9Y6Q3 | 7539 | 32 . . . 103 |
| ZFP57 | ZFP57 zinc finger protein | A0A1U9X8V5 | 346171 | 43 . . . 116 |
| ZFP69 | ZFP69 zinc finger protein | Q49AA0 | 339559 | 76 . . . 147 |
| ZFP69B | ZFP69 zinc finger protein B | Q9UJL9 | 65243 | 74 . . . 145 |
| ZFP82 | ZFP82 zinc finger protein | D3Y299 | 284406 | 5 . . . 77 |
| ZFP90 | ZFP90 zinc finger protein | J3QKQ8 | 146198 | 13 . . . 85 |
| ZFP92 | ZFP92 zinc finger protein | A6NM28 | 139735 | 13 . . . 85 |
| ZIK1 | zinc finger protein interacting | B3KS08 | 284307 | 27 . . . 136 |
| | with K protein 1 | | | |
| ZIM2 | zinc finger imprinted 2 | A0A024R4S8 | 23619 | 175 . . . 246 |
| ZIM3 | zinc finger imprinted 3 | Q96PE6 | 114026 | 8 . . . 80 |
| ZKSCAN1 | zinc finger with KRAB and SCAN | B3KNP9 | 7586 | 191 . . . 264 |
| | domains 1 | | | |
| ZKSCAN2 | zinc finger with KRAB and SCAN | Q63HK3 | 342357 | 227 . . . 300 |
| | domains 2 | | | |
| ZKSCAN3 | zinc finger with KRAB and SCAN | Q9BRR0 | 80317 | 214 . . . 274 |
| | domains 3 | | | |
| ZKSCAN4 | zinc finger with KRAB and SCAN | A0A0S2Z658 | 387032 | 221 . . . 317 |
| | domains 4 | | | |
| ZKSCAN5 | zinc finger with KRAB and SCAN | B3KNK8 | 23660 | 218 . . . 291 |
| | domains 5 | | | |
| ZKSCAN7 | zinc finger with KRAB and SCAN | A7MAY2 | 55888 | 102 . . . 176 |
| | domains 7 | | | |
| ZKSCAN8 | zinc finger with KRAB and SCAN | A0A024RCK7 | 7745 | 220 . . . 316 |
| | domains 8 | | | |
| ZNF10 | zinc finger protein 10 | P21506 | 7556 | 13 . . . 85 |
| ZNF100 | zinc finger protein 100 | Q8IYN0 | 163227 | 34 . . . 107 |
| ZNF101 | zinc finger protein 101 | Q8IZC7 | 94039 | 3 . . . 82 |
| ZNF107 | zinc finger protein 107 | A0A087WYK5 | 51427 | 3 . . . 76 |
| ZNF112 | zinc finger protein 112 | Q0VG01 | 7771 | 2 . . . 73 |
| ZNF114 | zinc finger protein 114 | Q8NC26 | 163071 | 5 . . . 85 |
| ZNF12 | zinc finger protein 12 | P17014 | 7559 | 7 . . . 79 |
| ZNF124 | zinc finger protein 124 | J3QKQ6 | 7678 | 12 . . . 77 |
| ZNF132 | zinc finger protein 132 | B3KQ54 | 7691 | 38 . . . 121 |
| ZNF133 | zinc finger protein 133 | P52736 | 7692 | 1 . . . 72 |
| ZNF135 | zinc finger protein 135 | P52742 | 7694 | 14 . . . 85 |
| ZNF136 | zinc finger protein 136 | P52737 | 7695 | 3 . . . 90 |
| ZNF138 | zinc finger protein 138 | A0A0A0MRG3 | 7697 | 3 . . . 89 |
| ZNF14 | zinc finger protein 14 | P17017 | 7561 | 3 . . . 76 |
| ZNF140 | zinc finger protein 140 | P52738 | 7699 | 5 . . . 77 |
| ZNF141 | zinc finger protein 141 | D6RIY0 | 7700 | 4 . . . 75 |
| ZNF154 | zinc finger protein 154 | A0A024R4Q0 | 7710 | 14 . . . 88 |
| ZNF155 | zinc finger protein 155 | Q12901 | 7711 | 8 . . . 78 |
| ZNF157 | zinc finger protein 157 | P51786 | 7712 | 26 . . . 98 |
| ZNF160 | zinc finger protein 160 | A0A024R4Q2 | 90338 | 8 . . . 79 |
| ZNF169 | zinc finger protein 169 | Q14929 | 169841 | 14 . . . 85 |
| ZNF17 | zinc finger protein 17 | P17021 | 7565 | 8 . . . 101 |
| ZNF175 | zinc finger protein 175 | Q9Y473 | 7728 | 26 . . . 98 |
| ZNF177 | zinc finger protein 177 | Q13360 | 7730 | 13 . . . 84 |
| ZNF18 | zinc finger protein 18 | B3KXT5 | 7566 | 211 . . . 283 |
| ZNF180 | zinc finger protein 180 | A0A0A0MP75 | 7733 | 45 . . . 118 |
| ZNF181 | zinc finger protein 181 | Q2M3W8 | 339318 | 4 . . . 76 |
| ZNF182 | zinc finger protein 182 | P17025 | 7569 | 26 . . . 98 |
| ZNF184 | zinc finger protein 184 | A0A024RCM2 | 7738 | 28 . . . 99 |
| ZNF189 | zinc finger protein 189 | A0A087X0K2 | 7743 | 1 . . . 85 |
| ZNF19 | zinc finger protein 19 | P17023 | 7567 | 14 . . . 85 |
| ZNF195 | zinc finger protein 195 | O14628 | 7748 | 4 . . . 75 |
| ZNF197 | zinc finger protein 197 | O14709 | 10168 | 216 . . . 289 |
| ZNF2 | zinc finger protein 2 | A0A087WYR5 | 7549 | 13 . . . 85 |
| ZNF20 | zinc finger protein 20 | P17024 | 7568 | 6 . . . 78 |
| ZNF202 | zinc finger protein 202 | A0A024R3M3 | 7753 | 236 . . . 308 |
| ZNF205 | zinc finger protein 205 | I0J0A3 | 7755 | 123 . . . 193 |
| ZNF208 | zinc finger protein 208 | M0QYK4 | 7757 | 3 . . . 75 |
| ZNF211 | zinc finger protein 211 | A0A0A0MTK1 | 10520 | 32 . . . 104 |
| ZNF212 | zinc finger protein 212 | A0A090N8N3 | 7988 | 141 . . . 219 |
| ZNF213 | zinc finger protein 213 | A0A0S2Z4L6 | 7760 | 202 . . . 292 |
| ZNF214 | zinc finger protein 214 | Q9UL59 | 7761 | 3 . . . 83 |
| ZNF215 | zinc finger protein 215 | Q9UL58 | 7762 | 163 . . . 237 |
| ZNF221 | zinc finger protein 221 | Q9UK13 | 7638 | 30 . . . 100 |
| ZNF222 | zinc finger protein 222 | Q9UK12 | 7673 | 8 . . . 80 |
| ZNF223 | zinc finger protein 223 | Q9UK11 | 7766 | 8 . . . 78 |
| ZNF224 | zinc finger protein 224 | Q9NZL3 | 7767 | 8 . . . 78 |
| ZNF225 | zinc finger protein 225 | Q9UK10 | 7768 | 8 . . . 78 |
| ZNF226 | zinc finger protein 226 | A0A024R0P4 | 7769 | 8 . . . 78 |
| ZNF227 | zinc finger protein 227 | Q658S5 | 7770 | 9 . . . 73 |
| ZNF229 | zinc finger protein 229 | A0A087WVZ7 | 7772 | 33 . . . 111 |
| ZNF23 | zinc finger protein 23 | B3KR55 | 7571 | 1 . . . 43 |
| ZNF230 | zinc finger protein 230 | Q9UIE0 | 7773 | 8 . . . 76 |
| ZNF233 | zinc finger protein 233 | A6NK53 | 353355 | 8 . . . 79 |
| ZNF234 | zinc finger protein 234 | A0A024R0N3 | 10780 | 8 . . . 90 |
| ZNF235 | zinc finger protein 235 | Q14590 | 9310 | 8 . . . 79 |
| ZNF248 | zinc finger protein 248 | A0A087WTK3 | 57209 | 8 . . . 79 |
| ZNF25 | zinc finger protein 25 | P17030 | 219749 | 7 . . . 79 |
| ZNF250 | zinc finger protein 250 | B3KNS9 | 58500 | 22 . . . 93 |
| ZNF251 | zinc finger protein 251 | Q9BRH9 | 90987 | 14 . . . 86 |
| ZNF253 | zinc finger protein 253 | Q75346 | 56242 | 4 . . . 75 |
| ZNF254 | zinc finger protein 254 | A0A087WZJ7 | 9534 | 1 . . . 43 |
| ZNF256 | zinc finger protein 256 | Q9Y2P7 | 10172 | 14 . . . 96 |
| ZNF257 | zinc finger protein 257 | Q9Y2Q1 | 113835 | 3 . . . 75 |
| ZNF26 | zinc finger protein 26 | P17031 | 7574 | 14 . . . 85 |
| ZNF263 | zinc finger protein 263 | D3DUC1 | 10127 | 217 . . . 289 |
| ZNF264 | zinc finger protein 264 | O43296 | 9422 | 13 . . . 85 |
| ZNF266 | zinc finger protein 266 | A0A024R7B6 | 10781 | 1 . . . 42 |
| ZNF267 | zinc finger protein 267 | Q14586 | 10308 | 4 . . . 75 |
| ZNF268 | zinc finger protein 268 | A0A075B6T9 | 10795 | 80 . . . 152 |
| ZNF273 | zinc finger protein 273 | Q14593 | 10793 | 36 . . . 108 |
| ZNF274 | zinc finger protein 274 | A0A0A0MR47 | 10782 | 1 . . . 43, |
| | | | | 244 . . . 318 |
| ZNF28 | zinc finger protein 28 | P17035 | 7576 | 8 . . . 81 |
| ZNF282 | zinc finger protein 282 | A0A090N8Y3 | 8427 | 197 . . . 271 |
| ZNF283 | zinc finger protein 283 | Q8N7M2 | 284349 | 72 . . . 146 |
| ZNF284 | zinc finger protein 284 | Q2VY69 | 342909 | 8 . . . 78 |
| ZNF285 | zinc finger protein 285 | B7ZLR9 | 26974 | 32 . . . 110 |
| ZNF286A | zinc finger protein 286A | B2RCD9 | 57335 | 45 . . . 111 |
| ZNF287 | zinc finger protein 287 | Q6PEZ3 | 57336 | 162 . . . 231 |
| ZNF3 | zinc finger protein 3 | B3KRP4 | 7551 | 33 . . . 106 |
| ZNF30 | zinc finger protein 30 | P17039 | 90075 | 13 . . . 85 |
| ZNF300 | zinc finger protein 300 | Q96RE9 | 91975 | 7 . . . 79 |
| ZNF302 | zinc finger protein 302 | A0A087WYF4 | 55900 | 4 . . . 76 |
| ZNF304 | zinc finger protein 304 | E7EQD3 | 57343 | 14 . . . 84 |
| ZNF311 | zinc finger protein 311 | A0A1U9X8W3 | 282890 | 63 . . . 135 |
| ZNF316 | zinc finger protein 316 | A6NFI3 | 100131017 | 157 . . . 229 |
| ZNF317 | zinc finger protein 317 | A0A024R775 | 57693 | 56 . . . 131 |
| ZNF320 | zinc finger protein 320 | A2RRD8 | 162967 | 8 . . . 80 |
| ZNF324 | zinc finger protein 324 | A0A024R4R8 | 25799 | 1 . . . 72 |
| ZNF324B | zinc finger protein 324B | Q6AW86 | 388569 | 1 . . . 72 |
| ZNF331 | zinc finger protein 331 | A0A024R4J5 | 55422 | 5 . . . 78 |
| ZNF333 | zinc finger protein 333 | B3KSN8 | 84449 | 93 . . . 165 |
| ZNF334 | zinc finger protein 334 | A0A087X1P4 | 55713 | 8 . . . 80 |
| ZNF337 | zinc finger protein 337 | A8K5C0 | 26152 | 12 . . . 83 |
| ZNF33A | zinc finger protein 33A | A0A0A0MR11 | 7581 | 29 . . . 101 |
| ZNF33B | zinc finger protein 33B | Q06732 | 7582 | 11 . . . 83 |
| ZNF34 | zinc finger protein 34 | A0A0C4DG42 | 80778 | 14 . . . 87 |
| ZNF343 | zinc finger protein 343 | A0A087WZQ2 | 79175 | 61 . . . 145 |
| ZNF347 | zinc finger protein 347 | A0A024R4L7 | 84671 | 8 . . . 80 |
| ZNF350 | zinc finger protein 350 | Q9GZX5 | 59348 | 7 . . . 79 |
| ZNF354A | zinc finger protein 354A | Q60765 | 6940 | 13 . . . 85 |
| ZNF354B | zinc finger protein 354B | Q96LW1 | 117608 | 13 . . . 85 |
| ZNF354C | zinc finger protein 354C | Q86Y25 | 30832 | 11 . . . 83 |
| ZNF37A | zinc finger protein 37A | A0A1B0GVV4 | 7587 | 7 . . . 79 |
| ZNF382 | zinc finger protein 382 | Q96SR6 | 84911 | 6 . . . 78 |
| ZNF383 | zinc finger protein 383 | Q8NA42 | 163087 | 5 . . . 77 |
| ZNF394 | zinc finger protein 394 | Q53GI3 | 84124 | 155 . . . 230 |
| ZNF398 | zinc finger protein 398 | A0A090N7S8 | 57541 | 142 . . . 214 |
| ZNF404 | zinc finger protein 404 | Q494X3 | 342908 | 5 . . . 78 |
| ZNF41 | zinc finger protein 41 | A0A024R1C4 | 7592 | 26 . . . 98 |
| ZNF416 | zinc finger protein 416 | Q9BWM5 | 55659 | 28 . . . 111 |
| ZNF417 | zinc finger protein 417 | M0R230 | 147687 | 14 . . . 87 |
| ZNF418 | zinc finger protein 418 | B7Z1P3 | 147686 | 26 . . . 112 |
| ZNF419 | zinc finger protein 419 | Q96HQ0 | 79744 | 27 . . . 98 |
| ZNF420 | zinc finger protein 420 | Q8TAQ5 | 147923 | 5 . . . 86 |
| ZNF425 | zinc finger protein 425 | A0A090N7U3 | 155054 | 9 . . . 80 |
| ZNF426 | zinc finger protein 426 | A0A024R7D7 | 79088 | 41 . . . 112 |
| ZNF429 | zinc finger protein 429 | Q86V71 | 353088 | 3 . . . 75 |
| ZNF43 | zinc finger protein 43 | A0A024R7P7 | 7594 | 1 . . . 69 |
| ZNF430 | zinc finger protein 430 | A8K360 | 80264 | 34 . . . 107 |
| ZNF431 | zinc finger protein 431 | A0A024R7Q8 | 170959 | 35 . . . 106 |
| ZNF432 | zinc finger protein 432 | A0A024R413 | 9668 | 8 . . . 79 |
| ZNF433 | zinc finger protein 433 | C9JQA6 | 163059 | 17 . . . 96 |
| ZNF436 | zinc finger protein 436 | Q9C0F3 | 80818 | 13 . . . 86 |
| ZNF439 | zinc finger protein 439 | A0A804HI69 | 90594 | 23 . . . 106 |
| ZNF44 | zinc finger protein 44 | F8W7T7 | 51710 | 3 . . . 90 |
| ZNF440 | zinc finger protein 440 | Q81Y18 | 126070 | 3 . . . 86 |
| ZNF441 | zinc finger protein 441 | Q8N8Z8 | 126068 | 3 . . . 79 |
| ZNF442 | zinc finger protein 442 | Q9H7R0 | 79973 | 28 . . . 115 |
| ZNF443 | zinc finger protein 443 | Q9Y2A4 | 10224 | 3 . . . 86 |
| ZNF445 | zinc finger protein 445 | P59923 | 353274 | 234 . . . 304 |
| ZNF446 | zinc finger protein 446 | Q9NWS9 | 55663 | 208 . . . 254 |
| ZNF45 | zinc finger protein 45 | Q02386 | 7596 | 8 . . . 78 |
| ZNF454 | zinc finger protein 454 | Q8N9F8 | 285676 | 13 . . . 84 |
| ZNF460 | zinc finger protein 460 | Q14592 | 10794 | 12 . . . 99 |
| ZNF461 | zinc finger protein 461 | B4DRP8 | 92283 | 5 . . . 77 |
| ZNF468 | zinc finger protein 468 | Q5VIY5 | 90333 | 8 . . . 80 |
| ZNF470 | zinc finger protein 470 | Q6ECI4 | 388566 | 22 . . . 94 |
| ZNF471 | zinc finger protein 471 | A0A024R4Q9 | 57573 | 13 . . . 85 |
| ZNF473 | zinc finger protein 473 | A0A024QZI1 | 25888 | 6 . . . 75 |
| ZNF479 | zinc finger protein 479 | Q96JC4 | 90827 | 16 . . . 87 |
| ZNF480 | zinc finger protein 480 | B7Z8E1 | 147657 | 27 . . . 115 |
| ZNF483 | zinc finger protein 483 | Q6P088 | 158399 | 169 . . . 241 |
| ZNF484 | zinc finger protein 484 | Q5JVG2 | 83744 | 7 . . . 78 |
| ZNF485 | zinc finger protein 485 | A0A024R7T5 | 220992 | 10 . . . 82 |
| ZNF 486 | zinc finger protein 486 | Q4G180 | 90649 | 53 . . . 124 |
| ZNF490 | zinc finger protein 490 | Q9ULM2 | 57474 | 57 . . . 132 |
| ZNF492 | zinc finger protein 492 | Q9P255 | 57615 | 1 . . . 43 |
| ZNF496 | zinc finger protein 496 | Q96IT1 | 84838 | 221 . . . 291 |
| ZNF500 | zinc finger protein 500 | Q60304 | 26048 | 223 . . . 247 |
| ZNF506 | zinc finger protein 506 | Q5JVG8 | 440515 | 4 . . . 75 |
| ZNF510 | zinc finger protein 510 | Q6NUI8 | 22869 | 45 . . . 117 |
| ZNF514 | zinc finger protein 514 | Q96K75 | 84874 | 1 . . . 72 |
| ZNF517 | zinc finger protein 517 | A0AV05 | 340385 | 14 . . . 85 |
| ZNF519 | zinc finger protein 519 | Q8TB69 | 162655 | 4 . . . 78 |
| ZNF525 | zinc finger protein 525 | Q8N782 | 170958 | 8 . . . 81 |
| ZNF527 | zinc finger protein 527 | Q8NB42 | 84503 | 13 . . . 85 |
| ZNF528 | zinc finger protein 528 | Q3MIS6 | 84436 | 8 . . . 79 |
| ZNF529 | zinc finger protein 529 | Q6P280 | 57711 | 38 . . . 119 |
| ZNF530 | zinc finger protein 530 | M0R1P0 | 348327 | 2 . . . 21 |
| ZNF534 | zinc finger protein 534 | Q1T7F5 | 147658 | 8 . . . 79 |
| ZNF540 | zinc finger protein 540 | Q8NDQ6 | 163255 | 5 . . . 77 |
| ZNF543 | zinc finger protein 543 | Q08ER8 | 125919 | 8 . . . 80 |
| ZNF544 | zinc finger protein 544 | B4DL50 | 27300 | 13 . . . 87 |
| ZNF546 | zinc finger protein 546 | B3KVL3 | 339327 | 33 . . . 105 |
| ZNF547 | zinc finger protein 547 | Q8IVP9 | 284306 | 11 . . . 82 |
| ZNF548 | zinc finger protein 548 | Q8NEK5 | 147694 | 8 . . . 79 |
| ZNF549 | zinc finger protein 549 | Q6P9A3 | 256051 | 27 . . . 140 |
| ZNF550 | zinc finger protein 550 | Q7Z398 | 162972 | 11 . . . 83 |
| ZNF551 | zinc finger protein 551 | Q7Z340 | 90233 | 29 . . . 100 |
| ZNF552 | zinc finger protein 552 | B7Z1H1 | 79818 | 10 . . . 86 |
| ZNF554 | zinc finger protein 554 | Q86TJ5 | 115196 | 44 . . . 148 |
| ZNF555 | zinc finger protein 555 | Q8NEP9 | 148254 | 3 . . . 77 |
| ZNF556 | zinc finger protein 556 | A0A0C4DGQ3 | 80032 | 4 . . . 77 |
| ZNF557 | zinc finger protein 557 | Q8N988 | 79230 | 35 . . . 107 |
| ZNF558 | zinc finger protein 558 | Q96NG5 | 148156 | 42 . . . 114 |
| ZNF559 | zinc finger protein 559 | A0A024R7B5 | 84527 | 13 . . . 89 |
| ZNF560 | zinc finger protein 560 | Q96MR9 | 147741 | 12 . . . 84, |
| | | | | 109 . . . 181 |
| ZNF561 | zinc finger protein 561 | Q8N587 | 93134 | 40 . . . 112 |
| ZNF562 | zinc finger protein 562 | A0A024R7A3 | 54811 | 40 . . . 120 |
| ZNF563 | zinc finger protein 563 | Q8TA94 | 147837 | 4 . . . 96 |
| ZNF564 | zinc finger protein 564 | Q8TBZ8 | 163050 | 3 . . . 83 |
| ZNF565 | zinc finger protein 565 | Q8N9K5 | 147929 | 45 . . . 117 |
| ZNF566 | zinc finger protein 566 | Q969W8 | 84924 | 5 . . . 77 |
| ZNF567 | zinc finger protein 567 | B2R956 | 163081 | 1 . . . 43 |
| ZNF568 | zinc finger protein 568 | A0A087WZ84 | 374900 | 48 . . . 119, |
| | | | | 137 . . . 208 |
| ZNF569 | zinc finger protein 569 | A0A024R0G4 | 148266 | 8 . . . 79 |
| ZNF57 | zinc finger protein 57 | A5HJR3 | 126295 | 3 . . . 76 |
| ZNF570 | zinc finger protein 570 | K7EP39 | 148268 | 69 . . . 141 |
| ZNF571 | zinc finger protein 571 | A0A024R0L0 | 51276 | 5 . . . 84 |
| ZNF573 | zinc finger protein 573 | Q86YE8 | 126231 | 27 . . . 100 |
| ZNF577 | zinc finger protein 577 | A0A024R4N6 | 84765 | 22 . . . 94 |
| ZNF578 | zinc finger protein 578 | Q96N58 | 147660 | 24 . . . 98 |
| ZNF582 | zinc finger protein 582 | A0A024R4P7 | 147948 | 6 . . . 77 |
| ZNF583 | zinc finger protein 583 | Q96ND8 | 147949 | 5 . . . 77 |
| ZNF584 | zinc finger protein 584 | B4DQS1 | 201514 | 8 . . . 80 |
| ZNF585A | zinc finger protein 585A | A0A0C4DFY4 | 199704 | 1 . . . 42 |
| ZNF585B | zinc finger protein 585B | Q52M93 | 92285 | 26 . . . 97 |
| ZNF586 | zinc finger protein 586 | Q9NXT0 | 54807 | 14 . . . 87 |
| ZNF587 | zinc finger protein 587 | Q96SQ5 | 84914 | 15 . . . 88 |
| ZNF587B | zinc finger protein 587B | E7ETH6 | 100293516 | 15 . . . 91 |
| ZNF589 | zinc finger protein 589 | Q86UQ0 | 51385 | 34 . . . 108 |
| ZNF595 | zinc finger protein 595 | A0A075B7G4 | 152687 | 3 . . . 78 |
| ZNF596 | zinc finger protein 596 | Q8TC21 | 169270 | 6 . . . 78 |
| ZNF597 | zinc finger protein 597 | Q96LX8 | 146434 | 14 . . . 72 |
| ZNF599 | zinc finger protein 599 | Q96NL3 | 148103 | 8 . . . 80 |
| ZNF600 | zinc finger protein 600 | A0A3B3IT03 | 162966 | 24 . . . 98 |
| ZNF605 | zinc finger protein 605 | Q86T29 | 100289635 | 6 . . . 77 |
| ZNF606 | zinc finger protein 606 | A0A024R4S7 | 80095 | 61 . . . 133 |
| ZNF607 | zinc finger protein 607 | Q96SK3 | 84775 | 5 . . . 78 |
| ZNF610 | zinc finger protein 610 | Q8N9Z0 | 162963 | 24 . . . 95 |
| ZNF611 | zinc finger protein 611 | Q8N823 | 81856 | 24 . . . 97 |
| ZNF613 | zinc finger protein 613 | Q6PF04 | 79898 | 7 . . . 78 |
| ZNF614 | zinc finger protein 614 | Q8N883 | 80110 | 7 . . . 79 |
| ZNF615 | zinc finger protein 615 | B4DH87 | 284370 | 12 . . . 84 |
| ZNF616 | zinc finger protein 616 | Q08AN1 | 90317 | 7 . . . 84 |
| ZNF620 | zinc finger protein 620 | Q6ZNG0 | 253639 | 10 . . . 82 |
| ZNF621 | zinc finger protein 621 | Q6ZSS3 | 285268 | 10 . . . 83 |
| ZNF624 | zinc finger protein 624 | Q9P2J8 | 57547 | 53 . . . 125 |
| ZNF626 | zinc finger protein 626 | Q68DY1 | 199777 | 4 . . . 75 |
| ZNF627 | zinc finger protein 627 | Q7L945 | 199692 | 3 . . . 80 |
| ZNF630 | zinc finger protein 630 | Q2M218 | 57232 | 7 . . . 79 |
| ZNF641 | zinc finger protein 641 | Q96N77 | 121274 | 108 . . . 181 |
| ZNF649 | zinc finger protein 649 | Q9BS31 | 65251 | 7 . . . 79 |
| ZNF658 | zinc finger protein 658 | A8K7Y5 | 26149 | 7 . . . 79 |
| ZNF662 | zinc finger protein 662 | Q6ZS27 | 389114 | 1 . . . 44 |
| ZNF665 | zinc finger protein 665 | Q9H7R5 | 79788 | 8 . . . 83 |
| ZNF667 | zinc finger protein 667 | Q5HYK9 | 63934 | 13 . . . 85 |
| ZNF669 | zinc finger protein 669 | Q96BR6 | 79862 | 89 . . . 176 |
| ZNF670 | zinc finger protein 670 | Q9BS34 | 93474 | 3 . . . 90 |
| ZNF671 | zinc finger protein 671 | Q8TAW3 | 79891 | 49 . . . 120 |
| ZNF674 | zinc finger protein 674 | A0A804HHU7 | 641339 | 7 . . . 79 |
| ZNF675 | zinc finger protein 675 | Q8TD23 | 171392 | 4 . . . 75 |
| ZNF676 | zinc finger protein 676 | Q8N7Q3 | 163223 | 1 . . . 43 |
| ZNF677 | zinc finger protein 677 | Q86XU0 | 342926 | 8 . . . 90 |
| ZNF679 | zinc finger protein 679 | Q8IYX0 | 168417 | 16 . . . 87 |
| ZNF680 | zinc finger protein 680 | Q8NEM1 | 340252 | 12 . . . 84 |
| ZNF681 | zinc finger protein 681 | Q96N22 | 148213 | 4 . . . 75 |
| ZNF682 | zinc finger protein 682 | B3KUX2 | 91120 | 1 . . . 43 |
| ZNF684 | zinc finger protein 684 | B3KSP5 | 127396 | 7 . . . 79 |
| ZNF688 | zinc finger protein 688 | A0A0S2Z633 | 146542 | 26 . . . 97 |
| ZNF689 | zinc finger protein 689 | Q96CS4 | 115509 | 29 . . . 100 |
| ZNF69 | zinc finger protein 69 | Q9UC07 | 7620 | 23 . . . 106 |
| ZNF695 | zinc finger protein 695 | Q8IW36 | 57116 | 4 . . . 87 |
| ZNF699 | zinc finger protein 699 | Q32M78 | 374879 | 18 . . . 90 |
| ZNF7 | zinc finger protein 7 | E9PIH3 | 7553 | 15 . . . 87 |
| ZNF700 | zinc finger protein 700 | A0A087WVH9 | 90592 | 26 . . . 107 |
| ZNF701 | zinc finger protein 701 | Q6NWZ7 | 55762 | 8 . . . 80 |
| ZNF705A | zinc finger protein 705A | Q6ZN79 | 440077 | 7 . . . 78 |
| ZNF705B | zinc finger protein 705B | P0CI00 | 100132396 | 7 . . . 78 |
| ZNF705D | zinc finger protein 705D | P0CH99 | 728957 | 7 . . . 78 |
| ZNF705EP | zinc finger protein 705E, | A8MWA4 | 100131539 | 7 . . . 78 |
| | pseudogene | | | |
| ZNF705G | zinc finger protein 705G | A8MUZ8 | 100131980 | 7 . . . 78 |
| ZNF707 | zinc finger protein 707 | Q96C28 | 286075 | 7 . . . 79 |
| ZNF708 | zinc finger protein 708 | P17019 | 7562 | 3 . . . 75 |
| ZNF709 | zinc finger protein 709 | Q8N972 | 163051 | 3 . . . 89 |
| ZNF713 | zinc finger protein 713 | Q8N859 | 349075 | 31 . . . 102 |
| ZNF714 | zinc finger protein 714 | A0A087WU35 | 148206 | 1 . . . 47 |
| ZNF716 | zinc finger protein 716 | A6NP11 | 441234 | 16 . . . 87 |
| ZNF717 | zinc finger protein 717 | C9J5W8 | 100131827 | 21 . . . 93 |
| ZNF718 | zinc finger protein 718 | Q3SXZ3 | 255403 | 4 . . . 75 |
| ZNF726 | zinc finger protein 726 | A6NNF4 | 730087 | 4 . . . 75 |
| ZNF727 | zinc finger protein 727 | A8MUV8 | 442319 | 4 . . . 75 |
| ZNF728 | zinc finger protein 728 | P0DKX0 | 388523 | 3 . . . 75 |
| ZNF729 | zinc finger protein 729 | A6NN14 | 100287226 | 12 . . . 84 |
| ZNF730 | zinc finger protein 730 | Q6ZMV8 | 100129543 | 4 . . . 75 |
| ZNF732 | zinc finger protein 732 | B4DXR9 | 654254 | 4 . . . 75 |
| ZNF735 | zinc finger protein 735 | P0CB33 | 730291 | 16 . . . 87 |
| ZNF736 | zinc finger protein 736 | B4DX44 | 728927 | 4 . . . 75 |
| ZNF737 | zinc finger protein 737 | O75373 | 100129842 | 4 . . . 75 |
| ZNF74 | zinc finger protein 74 | A8K5P3 | 7625 | 42 . . . 114 |
| ZNF746 | zinc finger protein 746 | Q6NUN9 | 155061 | 96 . . . 167 |
| ZNF747 | zinc finger protein 747 | A0A024QZG5 | 65988 | 37 . . . 140 |
| ZNF749 | zinc finger protein 749 | A0A024R4P6 | 388567 | 1 . . . 94 |
| ZNF75A | zinc finger protein 75a | A0A590UJE0 | 7627 | 235 . . . 307 |
| ZNF75D | zinc finger protein 75D | P51815 | 7626 | 235 . . . 318 |
| ZNF761 | zinc finger protein 761 | Q6IQ01 | 388561 | 8 . . . 70 |
| ZNF763 | zinc finger protein 763 | Q0D2J5 | 284390 | 3 . . . 86 |
| ZNF764 | zinc finger protein 764 | A0A024QZB2 | 92595 | 26 . . . 97 |
| ZNF765 | zinc finger protein 765 | Q7L2R6 | 91661 | 8 . . . 81 |
| ZNF766 | zinc finger protein 766 | Q5HY98 | 90321 | 9 . . . 80 |
| ZNF77 | zinc finger protein 77 | Q15935 | 58492 | 4 . . . 99 |
| ZNF772 | zinc finger protein 772 | Q68DY9 | 400720 | 27 . . . 98 |
| ZNF773 | zinc finger protein 773 | M0QYZ8 | 374928 | 14 . . . 85 |
| ZNF776 | zinc finger protein 776 | B4DSC6 | 284309 | 14 . . . 89 |
| ZNF777 | zinc finger protein 777 | Q9ULD5 | 27153 | 284 . . . 356 |
| ZNF778 | zinc finger protein 778 | A0A0A0MSW5 | 197320 | 42 . . . 113 |
| ZNF780A | zinc finger protein 780A | Q75290 | 284323 | 5 . . . 77 |
| ZNF780B | zinc finger protein 780B | A0A024R0P7 | 163131 | 5 . . . 77 |
| ZNF782 | zinc finger protein 782 | Q6ZMW2 | 158431 | 7 . . . 79 |
| ZNF783 | zinc finger protein 783 | Q6ZMS7 | 100289678 | 142 . . . 214 |
| ZNF785 | zinc finger protein 785 | A0A024QZC7 | 146540 | 29 . . . 100 |
| ZNF786 | zinc finger protein 786 | Q8N393 | 136051 | 8 . . . 80 |
| ZNF788P | zinc finger family member 788, | Q6ZQV5 | 388507 | 23 . . . 82 |
| | pseudogene | | | |
| ZNF789 | zinc finger protein 789 | Q5FWF6 | 285989 | 11 . . . 82 |
| ZNF79 | zinc finger protein 79 | B4DY65 | 7633 | 14 . . . 85 |
| ZNF790 | zinc finger protein 790 | B4DMI3 | 388536 | 5 . . . 76 |
| ZNF791 | zinc finger protein 791 | Q3KP31 | 163049 | 3 . . . 90 |
| ZNF792 | zinc finger protein 792 | Q3KQV3 | 126375 | 14 . . . 85 |
| ZNF793 | zinc finger protein 793 | Q6ZN11 | 390927 | 7 . . . 79 |
| ZNF799 | zinc finger protein 799 | Q96GE5 | 90576 | 3 . . . 86 |
| ZNF8 | zinc finger protein 8 | B3KS94 | 7554 | 24 . . . 96 |
| ZNF805 | zinc finger protein 805 | Q5CZA5 | 390980 | 12 . . . 84 |
| ZNF808 | zinc finger protein 808 | Q8N4W9 | 388558 | 24 . . . 98 |
| ZNF81 | zinc finger protein 81 | P51508 | 347344 | 20 . . . 92 |
| ZNF813 | zinc finger protein 813 | Q6ZN06 | 126017 | 8 . . . 81 |
| ZNF814 | zinc finger protein 814 | B7Z6K7 | 730051 | 15 . . . 91 |
| ZNF816 | zinc finger protein 816 | A0A024R4J0 | 125893 | 24 . . . 98 |
| ZNF816-ZNF321P | ZNF816-ZNF321P readthrough | A0A0X1KG74 | 100529240 | 24 . . . 97 |
| ZNF823 | zinc finger protein 823 | P16415 | 55552 | 3 . . . 97 |
| ZNF829 | zinc finger protein 829 | Q3KNS6 | 374899 | 34 . . . 106 |
| ZNF836 | zinc finger protein 836 | Q6ZNA1 | 162962 | 7 . . . 83 |
| ZNF84 | zinc finger protein 84 | P51523 | 7637 | 8 . . . 79 |
| ZNF844 | zinc finger protein 844 | B4DSG8 | 284391 | 3 . . . 86 |
| ZNF845 | zinc finger protein 845 | Q96IR2 | 91664 | 8 . . . 81 |
| ZNF846 | zinc finger protein 846 | Q147U1 | 162993 | 7 . . . 80 |
| ZNF85 | zinc finger protein 85 | Q03923 | 7639 | 3 . . . 75 |
| ZNF850 | zinc finger protein 850 | A0A087X0M6 | 342892 | 6 . . . 85 |
| ZNF852 | zinc finger protein 852 | Q6ZMS4 | 285346 | 8 . . . 82 |
| ZNF860 | zinc finger protein 860 | A6NHJ4 | 344787 | 24 . . . 99 |
| ZNF862 | zinc finger protein 862 | 060290 | 643641 | 10 . . . 77, |
| | | | | 332 . . . 404 |
| ZNF875 | zinc finger protein 875 | K7EPW3 | 284459 | 14 . . . 85 |
| ZNF878 | zinc finger protein 878 | C9JN71 | 729747 | 3 . . . 90 |
| ZNF879 | zinc finger protein 879 | B4DU55 | 345462 | 13 . . . 85 |
| ZNF880 | zinc finger protein 880 | Q6PDB4 | 400713 | 7 . . . 78 |
| ZNF888 | zinc finger protein 888 | P0CJ79 | 388559 | 8 . . . 79 |
| ZNF891 | zinc finger protein 891 | A8MT65 | 101060200 | 42 . . . 113 |
| ZNF90 | zinc finger protein 90 | Q03938 | 7643 | 4 . . . 75 |
| ZNF91 | zinc finger protein 91 | Q05481 | 7644 | 13 . . . 84 |
| ZNF92 | zinc finger protein 92 | Q03936 | 168374 | 3 . . . 75 |
| ZNF93 | zinc finger protein 93 | P35789 | 81931 | 4 . . . 75 |
| ZNF98 | zinc finger protein 98 | A6NK75 | 148198 | 13 . . . 84 |
| ZNF99 | zinc finger protein 99 | A8MXY4 | 7652 | 3 . . . 75 |
| ZSCAN25 | zinc finger and SCAN domain | Q6NSZ9 | 221785 | 231 . . . 259 |
| | containing 25 | | | |

TABLE 2
Exemplary sequences of human KRAB domain proteins

| | Alternative | | KRAB domain |
|---|---|---|---|
| Gene | Gene | | coordinates |
| Symbol | Symbol | SEQ ID NO: | (aa numbering) |
| ZFP1 | ZFP | 1 | 8 . . . 76 |
| ZFP14 | ZFP4 | 2 | 6 . . . 78 |
| ZFP82 | ZFP82 | 3 | 6 . . . 77 |
| ZIM2 | ZIM2 | 4 | 176 . . . 246 |
| ZIM3 | ZIM3 | 5 | 8 . . . 80 |
| ZNF140 | ZNF40 | 6 | 6 . . . 77 |
| ZNF175 | ZNF75 | 7 | 27 . . . 98 |
| ZNF18 | ZNF8 | 8 | 211 . . . 283 |
| ZNF184 | ZNF84 | 9 | 28 . . . 99 |
| ZNF189 | ZNF89 | 10 | 1 . . . 85 |
| ZNF213 | ZNF23 | 11 | 202 . . . 292 |
| ZNF214 | ZNF24 | 12 | 3 . . . 83 |
| ZNF224 | ZNF224 | 13 | 8 . . . 78 |
| ZNF250 | ZNF250 | 14 | 22 . . . 93 |
| ZNF254 | ZNF254 | 15 | 1 . . . 43 |
| ZNF257 | ZNF257 | 16 | 4 . . . 75 |
| ZNF264 | ZNF264 | 17 | 14 . . . 85 |
| ZNF273 | ZNF273 | 18 | 66 . . . 108 |
| ZNF30 | ZNF30 | 19 | 14 . . . 85 |
| ZNF320 | ZNF320 | 20 | 8 . . . 80 |
| ZNF324 | ZNF324 | 21 | 1 . . . 72 |
| ZNF331 | ZNF33 | 22 | 6 . . . 78 |
| ZNF33A | ZNF33A | 23 | 30 . . . 101 |
| ZNF34 | ZNF34 | 24 | 14 . . . 87 |
| ZNF350 | ZNF350 | 25 | 8 . . . 79 |
| ZNF354A | ZNF354A | 26 | 14 . . . 85 |
| ZNF37A | ZNF37A | 27 | 8 . . . 79 |
| ZNF394 | ZNF394 | 28 | 155 . . . 230 |
| ZNF41 | ZNF4 | 29 | 27 . . . 87 |
| ZNF416 | ZNF46 | 30 | 28 . . . 111 |
| ZNF419 | ZNF49 | 31 | 27 . . . 98 |
| ZNF436 | ZNF436 | 32 | 14 . . . 86 |
| ZNF45 | ZNF45 | 33 | 8 . . . 78 |
| ZNF490 | ZNF490 | 34 | 57 . . . 132 |
| ZNF528 | ZNF528 | 35 | 8 . . . 79 |
| ZNF543 | ZNF543 | 36 | 9 . . . 80 |
| ZNF547 | ZNF547 | 37 | 11 . . . 82 |
| ZNF554 | ZNF554 | 38 | 45 . . . 148 |
| ZNF557 | ZNF557 | 39 | 36 . . . 107 |
| ZNF566 | ZNF566 | 40 | 6 . . . 77 |
| ZNF582 | ZNF582 | 41 | 6 . . . 77 |
| ZNF595 | ZNF595 | 42 | 4 . . . 78 |
| ZNF596 | ZNF596 | 43 | 7 . . . 78 |
| ZNF610 | ZNF60 | 44 | 24 . . . 95 |
| ZNF669 | ZNF669 | 45 | 90 . . . 176 |
| ZNF675 | ZNF675 | 46 | 4 . . . 75 |
| ZNF677 | ZNF677 | 47 | 8 . . . 90 |
| ZNF680 | ZNF680 | 48 | 13 . . . 84 |
| ZNF729 | ZNF729 | 49 | 13 . . . 84 |
| ZNF764 | ZNF764 | 50 | 26 . . . 97 |
| ZNF785 | ZNF785 | 51 | 29 . . . 100 |
| ZNF8 | ZNF8 | 52 | 25 . . . 96 |
| ZNF816 | ZNF86 | 53 | 24 . . . 98 |
| ZNF98 | ZNF98 | 54 | 13 . . . 84 |

TABLE 3
Exemplary molecular heme therapeutic agents

| Drug (brand name) | Target gene | Target gene (inhibition) | Indication |
|---|---|---|---|
| Glasdegib maleate | SMO | SMO | Acute myeloid leukemia (AML) |
| (Daurismo) | | | |
| Fludarabine | | | B-cell chronic lymphocytic leukemia |
| phosphate | | | |
| (Fludara) | | | |
| Idelalisib (Zydelig) | PIK3KD | PIK3KD | Chronic lymphocytic leukaemia (CLL), B-cell |
| | | | non-Hodgkin's lymphoma, and small lymphocytic |
| | | | lymphoma (SLL) |
| Duvelisib | PIK3KD, PIK3KG | PIK3KD, PIK3KG | Chronic lymphocytic leukaemia (CLL), |
| (Copiktra) | | | follicular lymphoma (FL), |
| | | | and small lymphocytic lymphoma (SLL) |
| Venetoclax | BCL2 | BCL2 | Chronic lymphocytic leukemia |
| (Venclexta) | | | |
| Nilotinib HCl | ABL1 | ABL1 | Chronic myelogenous leukemia |
| (Tasigna) | | | |
| Imatinib mesylate | ABL1 | ABL1 | Chronic myeloid leukemia |
| (Gleevec) | | | |
| Dasatinib (Sprycel) | Tyrosine Kinase | | Chronic myeloid leukemia |
| Omacetaxine | Proteasome | PSMB5 | Chronic or accelerated-phase chronic |
| mepesuccinate | | | myeloid leukemia |
| (Synribo) | | | |
| Bosutinib | ABL1, SRC | ABL1, BTK, FYN, LYN, | Chronic, accelerated or blast phase Ph+ chronic |
| monohydrate | family | LCK, FGR, HCK, SRC | myelogenous leukemia |
| (Bosulif) | | | |
| Ponatinib HCl | BCR-ABL, KIT, | ABL1, KIT, RET, FLT3 | Chronic, accelerated or blast-phase chronic myeloid |
| (Iclusig) | RET and FLT3 | | leukemia |
| Vorinostat | HDAC | HDAC1, HDAC2, . . . | Cutaneous T-cell lymphoma |
| (Zolinza) | | HDAC11 | |
| Romidepsin | HDAC | HDAC1, HDAC2, . . . | Cutaneous T-cell lymphoma |
| (Istodax) | | HDAC11 | |
| Bexarotene | RXR | RXRA, RXRB, RXRG | Cutaneous T-cell lymphoma |
| (Targretin) | | | |
| Midostaurin | FLT3 and many | FLT3, . . . | FLT3-positive acute myeloid leukemia (AML) |
| (Rydapt) | others | | |
| Gilteritinib | FLT3 | FLT3 | FLT3-positive AML |
| fumarate | | | |
| (Xospata) | | | |
| Copanlisib 2HCI | PIK3KA, PIK3KD | PIK3KA, PIK3KD | Follicular lymphoma (FL) |
| (Aliqopa) | | | |
| Cladribine | Adenosine | ADA | Hairy cell leukemia and multiple sclerosis |
| (Leustatin) | Deaminase | | |
| Ivosidenib | IDH1 | IDH1 | IDH1-mutated acute myeloid leukemia (AML) |
| (Tibsovo) | | | |
| Enasidenib | IDH2 | IDH2 | IDH2-mutated acute myeloid leukemia (AML) |
| mesylate (Idhifa) | | | |
| Pentostatin | Adenosine | ADA | INFa-refractory hairy cell leukemia |
| (Nipent) | Deaminase | | |
| Zanubrutinib | BTK | BTK | Mantle cell lymphoma |
| (Brukinsa) | | | |
| Ibrutinib | BTK | BTK | Mantle cell lymphoma |
| (Imbruvica) | | | |
| Acalabrutinib | BTK | BTK | Mantle cell lymphoma |
| (Calquence) | | | |
| Carfilzomib | Proteasome | PSMB5 | Multiple myeloma |
| (Kyprolis) | | | |
| Selinexor (Xpovio) | XPO1 | XPO1 | Multiple myeloma |
| Panobinostat | HDAC | HDAC1, HDAC2, . . . | Multiple myeloma |
| lactate (Farydak) | | HDAC11 | |
| Ixazomib citrate | Proteasome | PSMB5 | Multiple myeloma |
| (Ninlaro) | | | |
| Melphalan | | | Multiple myeloma |
| flufenamide HCI | | | |
| (Pepaxto) | | | |
| Bortezomib | Proteasome | PSMB5 | Multiple myeloma |
| (Velcade) | | | |
| Pomalidomide | | | Multiple myeloma |
| (Pomalyst) | | | |
| Pralatrexate | | | Peripheral T cell lymphoma |
| (Folotyn) | | | |
| Belinostat | HDAC | HDAC1, HDAC2, . . . | Peripheral T-cell lymphoma |
| (Beleodaq) | | HDAC11 | |
| Asciminib HCI | ABL1 | ABL1 | Philadelphia chromosome-positive chronic myeloid |
| (Scemblix) | | | leukemia (Ph+ CML) |
| Teniposide | Topoisomerase II | TOP2A | Refractory childhood acute lymphoblastic leukemia |
| (Vumon) | | | |
| Clofarabine | | | Relapsed or refractory acute lymphoblastic leukemia |
| (Clolar) | | | |
| Umbralisib tosylate | PIK3KD, CK1e | PIK3KD, CSNK1E | Relapsed or refractory marginal zone lymphoma (MZL), |
| (Ukoniq) | | | follicular lymphoma |
| Nelarabine | | | T-cell acute lymphoblastic leukaemia and T-cell |
| (Arranon) | | | lymphoblastic lymphoma |

TABLE 4
Exemplary biologic heme therapeutic agents

| Drug (brand name) | Target gene | Target gene | Indication |
|---|---|---|---|
| Asparaginase erwinia | | | Acute lymphoblastic leukaemia (ALL), and lymphoblastic |
| chrysanthemi (Rylaze) | | | lymphoma (LBL), in patients allergic to E. coli-derived |
| | | | products |
| Asparaginase Erwinia | | | Acute lymphoblastic leukemia |
| chrysanthemi (Erwinaze) | | | |
| Calaspargase pegol (Asparlas) | | | Acute lymphoblastic leukemia (ALL) |
| Plerixafor (Mozobil) | CXCR4 | CXCR4 | Autologous transplantation in patients with |
| | | | non-Hodgkin's lymphoma and multiple myeloma |
| Inotuzumab ozogamicin | CD22 | CD22 | B cell precursor acute lymphoblastic leukemia (ALL) |
| (Besponsa) | | | |
| Alemtuzumab (Campath) | CD52 | CD52 | B-cell chronic lymphocytic leukemia |
| Loncastuximab tesirine | CD19 | CD19 | B-cell lymphoma |
| (Zynlonta) | | | |
| Ibritumomab tiuxetan (Zevalin) | CD20 | CD20 | B-cell non-Hodgkin's lymphoma |
| Rituximab (Rituxan) | CD20 | CD20 | B-cell non-Hodgkin's lymphoma |
| Blinatumomab (Blincyto) | CD19, CD3 | CD19, CD3 | B-cell precursor acute lymphoblastic leukemia (ALL) |
| Ofatumumab (Arzerra) | CD20 | CD20 | Chronic lymphocytic leukemia |
| Bendamustine HCl (Treanda) | | | Chronic lymphocytic leukemia |
| Denileukin diftitox (Ontak) | | | Cutaneous T-cell lymphoma |
| Polatuzumab vedotin (Polivy) | CD79b | CD79b | Diffuse large B-cell lymphoma |
| Tafasitamab (Monjuvi) | CD19 | CD19 | Diffuse large B-cell lymphoma (DLBCL) |
| Moxetumomab pasudotox | CD22 | CD22 | Hairy cell leukemia |
| (Lumoxiti) | | | |
| Brentuximab vedotin (Adcetris) | CD30 | CD30 | Hodgkin's lymphoma; systemic anaplastic large cell |
| | | | lymphoma |
| Belantamab mafodotin (Blenrep) | BCMA | BCMA | Multiple myeloma |
| Daratumumab (Darzalex) | CD38 | CD38 | Multiple myeloma |
| Isatuximab (Sarclisa) | CD38 | CD38 | Multiple myeloma |
| Elotuzumab (Empliciti) | SLAMF7 | SLAMF7 | Multiple myeloma |
| Sargramostim (Leukine) | GM-CSF | | Myeloid reconstitution after PBPC/bone marrow |
| | receptor | | transplantation, or with NHL, ALL, HL |
| Tositumomab and iodine I-131 | CD20 | CD20 | Non-Hodgkin's lymphoma |
| tositumomab (Bexxar) | | | |
| Pegaspargase (Oncaspar) | | | Pediatric acute lymphoblastic leukaemia |
| Obinutuzumab (Gazyva) | CD20 | CD20 | Previously untreated chronic lymphocytic leukemia |
| Gemtuzumab ozogamicin | CD33 | CD33 | Relapsed or refractory CD33-positive acute myeloid |
| (Mylotarg) | | | leukemia (AML) |

TABLE 5
Exemplary Myeloid neoplasms

| | | |
|---|---|---|
| Myeloproliferative | chronic myeloid leukemia, BCR-ABL1-positive | |
| neoplasms | chronic neutrophilic leukemia | |
| | polycythemia vera | |
| | primary myelofibrosis | |
| | essential thrombocythemia | |
| | chronic eosinophilic leukemia, not otherwise | |
| | specified | |
| | myeloproliferative neoplasm, unclassifiable | |
| Mastocytosis | cutaneous mastocytosis | |
| | systemic mastocytosis | indolent systemic mastocytosis |
| | | smoldering systemic mastocytosis |
| | | systemic mastocytosis with an associated |
| | | hematological neoplasm |
| | | aggressive systemic mastocytosis |
| | | mast cell leukemia |
| | mast cell sarcoma | |
| myeloid/lymphoid | myeloid/lymphoid neoplasms | |
| neoplasms with | with PDGFRA rearrangement | |
| eosinophilia and gene | myeloid/lymphoid neoplasms | |
| rearrangement | with PDGFRB rearrangement | |
| | myeloid/lymphoid neoplasms | |
| | with FGFR1 rearrangement | |
| | myeloid/lymphoid neoplasms with PCM1- | |
| | JAK2 (provisional entity) | |
| myelodysplastic/ | chronic myelomonocytic leukemia | |
| myeloproliferative | atypical chronic myeloid leukemia, BCR-ABL1- | |
| neoplasms | negative | |
| | juvenile myelomonocytic leukemia | |
| | myelodysplastic/myeloproliferative neoplasm with | |
| | ring sideroblasts and thrombocytosis | |
| | myelodysplastic/myeloproliferative neoplasm, | |
| | unclassifiable | |
| myelodysplastic | myelodysplastic syndrome with single lineage | |
| syndromes | dysplasia | |
| | myelodysplastic syndrome with multilineage | |
| | dysplasia | |
| | myelodysplastic syndrome with ring sideroblasts | myelodysplastic syndrome with ring sideroblasts and |
| | | single lineage dysplasia |
| | | myelodysplastic syndrome with ring sideroblasts and |
| | | multilineage dysplasia |
| | myelodysplastic syndrome with excess blasts | |
| | myelodysplastic syndrome with isolated del(5q) | |
| | myelodysplastic syndrome, unclassifiable | |
| | refractory cytopenia of childhood (provisional | |
| | entity) | |
| myeloid | myeloid neoplasms with germline predisposition | acute myeloid leukemia with |
| neoplasms with | without a preexisting disorder or organ dysfunction | germline CEBPA mutation |
| germline | | myeloid neoplasms with germline DDX41 mutation |
| predisposition | myeloid neoplasms with germline predisposition | myeloid neoplasms with germline RUNX1 mutation |
| | and preexisting platelet disorders | myeloid neoplasms with germline ANKRD26 mutation |
| | | myeloid neoplasms with germline ETV6 mutation |
| | myeloid neoplasms with germline predisposition | myeloid neoplasms with germline GATA2 mutation |
| | and other organ dysfunction | myeloid neoplasms associated with bone marrow |
| | | failure syndromes |
| | | myeloid neoplasms associated with telomere biology |
| | | disorders |
| | | juvenile myelomonocytic leukemia associated |
| | | with neurofibromatosis, Noonan syndrome or Noonan |
| | | syndrome-like disorders |
| | | myeloid neoplasms associated with Down syndrome |
| acute myeloid | acute myeloid leukemia with recurrent genetic | acute myeloid leukemia with |
| leukemia and | abnormalities | t(8; 21)(q22; q22.1); RUNX1-RUNX1T1 |
| related | | acute myeloid leukemia with inv(16)(p13.1q22) or |
| neoplasms | | t(16; 16)(p13.1; q22); CBFB-MYH11 |
| | | acute promyelocytic leukemia with PML-RARA |
| | | acute myeloid leukemia with |
| | | t(9; 11)(p21.3; q23.3); MLLT3-KMT2A |
| | | acute myeloid leukemia with t(6; 9)(p23; q34.1); DEK- |
| | | NUP214 |
| | | acute myeloid leukemia with inv(3)(q21.3q26.2) or |
| | | t(3; 3)(q21.3; q26.2); GATA2, MECOM |
| | | acute myeloid leukemia (megakaryoblastic) with |
| | | t(1; 22)(p13.3; q13.3); RBM15-MKL1 |
| | | acute myeloid leukemia with BCR-ABL1 (provisional |
| | | entity) |
| | | acute myeloid leukemia with mutated NPM1 |
| | | acute myeloid leukemia with biallelic mutations |
| | | of CEBPA |
| | | acute myeloid leukemia with |
| | | mutated RUNX1 (provisional entity) |
| | acute myeloid leukemia with myelodysplasia- | |
| | related changes | |
| | therapy-related myeloid neoplasms | |
| | acute myeloid leukemia, not otherwise specified | acute myeloid leukemia with minimal differentiation |
| | | acute myeloid leukemia without maturation |
| | | acute myeloid leukemia with maturation |
| | | acute myelomonocytic leukemia |
| | | acute monoblastic/monocytic leukemia |
| | | pure erythroid leukemia |
| | | acute megakaryoblastic leukemia |
| | | acute basophilic leukemia |
| | | acute panmyelosis with myelofibrosis |
| | myeloid sarcoma | |
| | myeloid proliferations related to Down syndrome | transient abnormal myelopoiesis |
| | | myeloid leukemia associated with Down syndrome |
| blastic | | |
| plasmacytoid | | |
| dendritic cell | | |
| neoplasm | | |
| acute | acute undifferentiated leukemia | |
| leukemias of | mixed phenotype acute leukemia with | |
| ambiguous | t(9;22)(q34.1;q11.2); BCR-ABL1 | |
| lineage | mixed phenotype acute leukemia with | |
| | t(v;11q23.3); KMT2A rearranged | |
| | mixed phenotype acute leukemia, B/myeloid, not | |
| | otherwise specified | |
| | mixed phenotype acute leukemia, T/myeloid, not | |
| | otherwise specified | |

TABLE 6
Exemplary lymphoid neoplasms

| | | | |
|---|---|---|---|
| precursor | B-lymphoblastic | B-lymphoblastic leukemia/lymphoma, not | |
| lymphoid | leukemia/lymphoma | otherwise specified | |
| neoplasms | | B-lymphoblastic leukemia/lymphoma with | B-lymphoblastic leukemia/lymphoma with |
| | | recurrent genetic abnormalities | t(9;22)(q34.1;q11.2); BCR-ABL1 |
| | | | B-lymphoblastic leukemia/lymphoma with |
| | | | t(v;11q23.3); KMT2A rearranged |
| | | | B-lymphoblastic leukemia/lymphoma with |
| | | | t(12;21)(p13.2;q22.1); ETV6-RUNX1 |
| | | | B-lymphoblastic leukemia/lymphoma with |
| | | | hyperdiploidy |
| | | | B-lymphoblastic leukemia/lymphoma with |
| | | | hypodiploidy |
| | | | B-lymphoblastic leukemia/lymphoma with |
| | | | t(5;14)(q31.1;q32.3); IL3-IGH |
| | | | B-lymphoblastic leukemia/lymphoma with |
| | | | t(1;19)(q23;p13.3); TCF3-PBX1 |
| | | | B-lymphoblastic leukemia/lymphoma, |
| | | | BCR-ABL1-like (provisional entity) |
| | | | B-lymphoblastic leukemia/lymphoma with |
| | | | iAMP21 (provisional entity) |
| | T-lymphoblastic | early T-cell precursor lymphoblastic leukemia | |
| | leukemia/lymphoma | (provisional entity) | |
| | natural killer (NK) cell | | |
| | lymphoblastic | | |
| | leukemia/lymphoma | | |
| | (provisional entity) | | |
| mature | chronic lymphocytic | | |
| B-cell | leukemia/small lymphocytic | | |
| neoplasms | lymphoma | | |
| | monoclonal B-cell | | |
| | lymphocytosis | | |
| | B-cell prolymphocytic | | |
| | leukemia | | |
| | splenic marginal zone | | |
| | lymphoma | | |
| | hairy cell leukemia | | |
| | splenic B-cell | splenic diffuse red pulp small B-cell lymphoma | |
| | lymphoma/leukemia, | (provisional entity) | |
| | unclassifiable | hairy cell leukemia-variant (provisional entity) | |
| | lymphoplasmacytic | Waldenstrom macroglobulinemia | |
| | lymphoma | | |
| | IgM monoclonal | | |
| | gammopathy of | | |
| | undetermined significance | | |
| | heavy-chain diseases | μ heavy-chain disease | |
| | | γ heavy-chain disease | |
| | | α heavy-chain disease | |
| | plasma cell neoplasms | non-IgM monoclonal gammopathy of | |
| | | undetermined significance (IgG/A) | |
| | | plasma cell myeloma (a.k.a. multiple myeloma) | |
| | | plasma cell myeloma variants | smoldering (asymptomatic) plasma cell |
| | | | myeloma |
| | | | non-secretory myeloma |
| | | | plasma cell leukemia |
| | | plasmacytoma | solitary plasmacytoma of bone |
| | | | extraosseous plasmacytoma |
| | | monoclonal immunoglobulin deposition diseases | primary amyloidosis |
| | | | light chain and heavy chain deposition |
| | | | diseases |
| | | plasma cell neoplasms with associated | POEMS syndrome |
| | | paraneoplastic syndrome | TEMPI syndrome |

extranodal marginal zone lymphoma of mucosa-associated lymphoid tissue
(MALT lymphoma)

| | nodal marginal zone | pediatric nodal marginal zone lymphoma |
|---|---|---|
| | lymphoma | (provisional entity) |
| | follicular lymphoma | testicular follicular lymphoma |
| | | in situ follicular neoplasia |
| | | duodenal-type follicular lymphoma |
| | pediatric-type follicular | |
| | lymphoma | |
| | large B-cell lymphoma | |
| | with IRF4 rearrangement | |
| | (provisional entity) | |
| | primary cutaneous follicle | |
| | center lymphoma | |
| | mantle cell lymphoma | leukemic non-nodal mantle cell lymphoma |
| | | in situ mantle cell neoplasia |
| | diffuse large B-cell | germinal center B-cell type |
| | lymphoma, not otherwise | |
| | specified | activated B-cell type |
| | T-cell/histiocyte-rich large | |
| | B-cell lymphoma | |
| | primary diffuse large B-cell | |
| | lymphoma of the central | |
| | nervous system | |
| | primary cutaneous diffuse | |
| | large B-cell lymphoma, leg | |
| | type | |
| | EBV-positive diffuse large | |
| | B-cell lymphoma, not | |
| | otherwise specified | |
| | EBV-positive | |
| | mucocutaneous ulcer | |
| | (provisional entity) | |
| | diffuse large B-cell | fibrin-associated diffuse large B-cell lymphoma |
| | lymphoma associated with | |
| | chronic inflammation | |
| | lymphomatoid | |
| | granulomatosis | |
| | primary mediastinal | |
| | (thymic) large B-cell | |
| | lymphoma | |
| | intravascular large B-cell | |
| | lymphoma | |
| | ALK-positive large B-cell | |
| | lymphoma | |
| | plasmablastic lymphoma | |
| | primary effusion lymphoma | |
| | HHV8-associated | multicentric Castleman disease |
| | lymphoproliferative | HHV8-positive diffuse large B-cell lymphoma, not otherwise specified (provisional entity) |
| | disorders | HHV8-positive germinotropic lymphoproliferative |
| | | disorder |
| | Burkitt lymphoma | |
| | Burkitt-like lymphoma with | |
| | 11q aberration (provisional | |
| | entity) | |
| | high-grade B-cell | high-grade B-cell lymphoma |
| | lymphoma | with MYC and BCL2 and/or BCL6 rearrangements |
| | | high-grade B-cell lymphoma, not otherwise |
| | | specified |
| | B-cell lymphoma, unclassifiable, with features intermediate between diffuse | |
| | large B-cell lymphoma and classical Hodgkin lymphoma | |
| mature | T-cell prolymphocytic | |
| T-and | leukemia | |
| NK-cell | T-cell large granular | |
| neoplasms | lymphocytic leukemia | |
| | chronic lymphoproliferative | |
| | disorder of NK cells | |
| | (provisional entity) | |
| | aggressive NK-cell | |
| | leukemia | |
| | EBV-positive T-cell and | systemic EBV-positive T-cell lymphoma of |
| | NK-cell lymphoproliferative | childhood |
| | diseases of childhood | chronic active EBV infection of T-and NK-cell |
| | | type, systemic form |
| | | hydroa vacciniforme-like lymphoproliferative |
| | | disorder |
| | | severe mosquito bite allergy |
| | adult T-cell | |
| | leukemia/lymphoma | |
| | extranodal NK-/T-cell | |
| | lymphoma, nasal type | |
| | intestinal T-cell lymphoma | enteropathy-associated T-cell lymphoma |
| | | monomorphic epitheliotropic intestinal T-cell |
| | | lymphoma |
| | | intestinal T-cell lymphoma, not otherwise |
| | | specified |
| | | indolent T-cell lymphoproliferative disorder of the gastrointestinal tract (provisional entity) |
| | hepatosplenic T-cell | |
| | lymphoma | |
| | subcutaneous panniculitis- | |
| | like T-cell lymphoma | |
| | mycosis fungoides | |
| | Sézary syndrome | |
| | primary cutaneous CD30+ | lymphomatoid papulosis |
| | T-cell lymphoproliferative | |
| | disorders | primary cutaneous anaplastic large cell lymphoma |
| | primary cutanoue | primary cutaneous yo T-cell lymphoma |
| | peripheral T-cell | primary cutaneous CD8+ aggressive epidermotropic cytotoxic T-cell lymphoma |
| | lymphomas, rare subtypes | (provisional entity) |
| | | primary cutaneous acral CD8+ T-cell lymphoma |
| | | (provisional entity) |
| | | primary cutaneous CD4+ small/medium T-cell lymphoproliferative disorder (provisional entity) |
| | peripheral T-cell | |
| | lymphoma, not otherwise | |
| | specified | |
| | angioimmunoblastic T-cell | angioimmunoblastic T-cell lymphoma |
| | lymphoma and other nodal | follicular T-cell lymphoma (provisional entity) |
| | lymphomas of T follicular | nodal peripheral T-cell lymphoma with T follicular |
| | helper cell origin | helper phenotype (provisional entity) |
| | anaplastic large-cell | anaplastic large-cell lymphoma, ALK-positive |
| | lymphoma | anaplastic large-cell lymphoma, ALK-negative |
| | breast implant-associated | |
| | anaplastic large-cell | |
| | lymphoma (provisional | |
| | entity) | |
| Hodgkin | nodular lymphocyte | |
| lymphoma | predominant Hodgkin | |
| | lymphoma | |
| | classical Hodgkin | nodular sclerosis classical Hodgkin lymphoma |
| | lymphoma | lymphocyte-rich classical Hodgkin lymphoma |
| | | mixed cellularity classical Hodgkin lymphoma |
| | | lymphocyte-depleted classical Hodgkin lymphoma |
| posttransplant | plasmacytic hyperplasia | |
| lymphoproliferative | PTLD | |
| disorders | infectious mononucleosis | |
| (PTLD) | PTLD | |
| | florid follicular hyperplasia | |
| | PTLD | |
| | polymorphic PTLD | |
| | monomorphic PTLD | monomorphic B-cell PTLD |
| | | monomorphic T/NK-cell PTLD |
| | classical Hodgkin | |
| | lymphoma PTLD | |

TABLE 7
Exemplary histiocytic and dendritic cell neoplasms

| | |
|---|---|
| histiocytic sarcoma | |
| tumors derived from Langerhans cells | Langerhans cell histiocytosis |
| | Langerhans cell sarcoma |
| indeterminate dendritic cell tumor | |
| interdigitating dendritic cell sarcoma | |
| follicular dendritic cell sarcoma | inflammatory pseudotumor-like |
| | follicular/fibroblastic dendritic cell sarcoma |
| fibroblastic reticular cell tumor | |
| disseminated juvenile xanthogranuloma | |
| Erdheim-Chester disease | |

TABLE 8
Exemplary different classes and subtypes of heme malignancies

| Class | Subtype |
|---|---|
| Acute Leukemias | Acute myeloid leukaemia in adult |
| | Acute myeloid leukaemia in children |
| | Acute lymphoid leukaemia in adult |
| | Acute lymphoid leukaemia in children |
| Myelodysplastic and myeloproliferative syndromes | Myelodysplastic and myeloproliferative syndromes |
| Chronic Myeloproliferative Syndromes | Chronic myeloid leukaemia |
| | Primary myelofibrosis |
| | Essential thrombocythemia |
| | Polycythemia vera |
| Lymphoproliferative Syndromes | Hodgkin's Lymphoma |
| | Non-Hodgkin's Lymphomas |
| | Chronic Lymphocytic Leukemia |
| Monoclonal gammopathies | Monoclonal gammopathy of unknown significance |
| | Multiple myeloma |
| | Waldenstrom's Macroglobulinemia |
| | Heavy chain disease |
| | Primary amyloidosis |

TABLE 9A
Gene target and associated sgRNA sequences

| | Orientation (relative | sg RNA | SEQ ID | |
|---|---|---|---|---|
| Gene | to transcription) | Position | NO: | sgRNA Sequence |
| ABL1 to | sense or antisense/ | provided in | 55-459 | provided in sequence |
| ZRSR2 | provided in | sequence | | listing for each |
| | sequence listing | listing | | SEQ ID NO: 55-459 |

TABLE 9B
Gene target and associated sgRNA sequences

| | Location | Orientation | | SEQ | |
|---|---|---|---|---|---|
| | (start to end; | (relative to | sgRNA | ID | |
| Gene | relative to TSS) | transcription) | Position | NO: | sgRNA Sequence |
| ABL1 to | provided in | sense or | provided in | 460-1669 | provided in sequence |
| WRN | sequence listing | antisense/ | sequence | | listing for each |
| | | provided in | listing | | SEQ ID NO: 460-1669 |
| | | sequence listing | | | |

TABLE 9C
Kinase gene target and associated sgRNA sequences

| | Location | Orientation | | SEQ | |
|---|---|---|---|---|---|
| | (start to end; | (relative to | sgRNA | ID | |
| Gene | relative to TSS) | transcription) | Position | NO: | sgRNA Sequence |
| AAK1 to | provided in | sense or | provided in | 1670-8119 | provided in sequence |
| ZAP70 | sequence listing | antisense/ | sequence | | listing for each |
| | | provided in | listing | | SEQ ID NO: 1670-8119 |
| | | sequence listing | | | |

TABLE 9D
IgSF gene target and associated sgRNA sequences

| | Location | Orientation | | SEQ | |
|---|---|---|---|---|---|
| | (start to end; | (relative to | sgRNA | ID | |
| Gene | relative to TSS) | transcription) | Position | NO: | sgRNA Sequence |
| A1BG to | provided in | sense or | provided in | 8120- | provided in sequence |
| ZPBP2 | sequence | antisense/ | sequence | 12949 | listing for each |
| | listing | provided in | listing | | SEQ ID NO: 8120-12949 |
| | | sequence | | | |
| | | listing | | | |

TABLE 10A
Exemplary approved heme therapeutic agents with associated targets or gene products

| | FDA approved | |
|---|---|---|
| Gene | drugs (as of 2021) | Drugs targeting or interacting with gene products (as of 2021) |
| ABL1 | Imatinib mesylate | IMATINIB\IMATINIB MESYLATE\dasatinib\ponatinib\Imatinib |
| | (Gleevec); Nilotinib | (Gleevec)\Bosutinib\AZD0530\Flumatinib\DCC-2036\KW-2449\MC-2001\G- |
| | HCl (Tasigna); | 1128\Adenosine triphosphate\Radotinib1923288-90-8\PONATINIB |
| | Bosutinib | HYDROCHLORIDE\REGORAFENIB\BAFETINIBIAT9283\nilotinib\homoharringtonine\ |
| | monohydrate | busulfan\vincristine\Dasatinib\Ponatinib\Nilotinib\Bosutinib\Asciminib\Imatinib\Bosutinib |
| | (Bosulif); Ponatinib | (Bosulif)\Dasatinib (Sprycel)\Nilotinib (Tasigna)\Ponatinib (Iclusig)\Imatinib |
| | HCl (Iclusig); | Mesylate\Axitinib (VEGFR inhibitor)\Bosutinib (BCR-ABL inhibitor 3rd gen)\ |
| | Asciminib HCl | Cabozantinib (Pan-kinase inhibitor)\Crizotinib (ALK inhibitor)\Dasatinib (BCR-ABL |
| | (Scemblix) | inhibitor 2nd gen)\Dasatinib + Venetoclax (BCR-ABL inhibitor 2nd gen + BCL2 inhibitor)\ |
| | | Foretinib¬† (MET inhibitor)\Imatinib (BCR-ABL inhibitor 1st gen&KIT inhibitor)\Nilotinib |
| | | (BCR-ABL inhibitor 2nd gen)\Ponatinib (BCR-ABL inhibitor 3rd gen&Pan-TK inhibitor)\ |
| | | Vandetanib (Pan-TK inhibitor)\Nilotinib nanoparticle formulation\XL- |
| | | 228\SARACATINIB\STI571\ABL 001\AN-019\NPB-001-056\Adaphostin\PMID25656651- |
| | | Compound-11a\PMID25656651-Compound-11b\PMID25656651-Compound- |
| | | 11c\PMID25656651-Compound-5\ON-044580\Imatinib sublingual |
| ALK | Crizotinib (Xalkori); | Gilteritinib\Alectinib\Brigatinib\Ceritinib\Crizotinib\Lorlatinib\Ensartinib\AP26113\PF- |
| | Ceritinib (Zykadia); | 06463922\RXDX 101\TPX-0005\TSR-011\X-396\ASP3026\Benzimidazole derivative |
| | Alectinib HCl | 6\PMID28270010-Compound-FIG. 21-b\CEP-18050\CEP-28122\CRL-37212\NMS- |
| | (Alecensa); | E628\ASP 3026\CHS424802\Ceritinib\Alectinib\Lorlatinib\Brigatinib\Alectinib |
| | Brigatinib | (Alecensa)\Brigatinib (Alunbrig)\Ceritinib (Zykadia)\Crizotinib (Xalkori)\ |
| | (Alunbrig); | TAE684\Luminespib\Pemetrexed\Erlotinib\WHI-P154\2\4-pyrimidinediamine\ |
| | Lorlatinib | Retaspimycin Hydrochloride\Alvespimycin\AZD3463\Tanespimycin\Entrectinib\ALK |
| | (Lorbrena); | inhibitor + IGF1R inhibitors\ALK inhibitor + MEK inhibitors\ALK inhibitor + SRC |
| | Entrectinib | inhibitors\ALK inhibitors\HSP90 inhibitors\novel ALK inhibitors\Alectinib (ALK |
| | (Rozlytrek) | inhibitor)\Brigatinib (Pan-TK inhibitor)\Ceritinib (ALK inhibitor)\Crizotinib (ALK inhibitor)\ |
| | | Entrictinib (Pan-TK inhibitor)\Lorlatinib (ALK&ROS1 inhibitor) |
| AR | Bicalutamide | Enzalutamide\Apalutamide\Nilutamide\ARN- |
| | (Casodex); | 509\Bicalutamide\Cyproterone\Dromostanolone\Fluoxymesterone\Flutamide\Hydroxyflutamide\ |
| | Nilutamide | AZD5312\Liproca Depot\TOK-001\APC-100\ONC1-13B\AZD-3514\RAD- |
| | (Nilandron); | 140\TAS3681\LG-2293\Andromustine\ASC-JMZ1\CH-4933468\DL-3\EPI-001\GTX- |
| | Enzalutamide | 027\SARMs\Selective androgen receptor degraders\SiRNA therapeutic\Lauriad/tablet\ |
| | (Xtandi); | prostate cancer\SX-ARPC\Darolutamide\BAY 86-5044\EZN-4176\ISIS-AR\TRC- |
| | Apalutamide | 253\WAY-255348\Hydroxyflutamide intraprostatic\Research programme: anticancer |
| | (Erleada); | therapeutics\ISIS 5132\DANAZOL\ENOBOSARM\2\5\7\8-PENTAMETHYL-6- |
| | Darolutamide | CHROMANOL\ODM- |
| | (Nubeqa); | 201\METHYLTESTOSTERONE\NANDROLONE\OXANDROLONE\DEHYDROEPIANDR |
| | Clascoterone | OSTERONE\DROSPIRENONE\NANDROLONE |
| | (Winlevi) | DECANOATE\OXYMETHOLONE\ANDARINE\METHYLTRIENOLONE\Bicalutamide\ |
| | | Dactolisib\Enzalutamide\Abiraterone\Flutamide\Cyproterone Acetate\AR inhibitor next |
| | | gens\AR inhibitors (Bicalutamide\Orterone\4OHtestosteroneletc)\Enzalutamide (AR |
| | | inhibitor)\Enzalutamide (AR inhibitor)\Mitotane |
| ARAF | Sorafenib tosylate | REGORAFENIB\RG7304\SORAFENIB\BMS-908662\ENCORAFENIB\Cobimetinib\ |
| | (Nexavar) | Trametinib\Irinotecan\Vemurafenib\Sorafenib (Pan-TK |
| | | inhibitor)\MLN2480\LY3009120\RO-5126766 |
| BCL2 | Venetoclax | Venetoclax\Navitoclax\GDC-0199\Oral paclitaxel\Taxol\Liposomal encapsulated |
| | (Venclexta) | paclitaxel (LEP)\Oblimersen\RG7601\Taxol/Paraplatin/Herceptin\ABT- |
| | | 263\Gossypol\Obatoclax\PI-88/Taxotere\AI-850\APG- |
| | | 1252\BCL201\Irofulven/Taxotere\Apogossypol\WL-276\PNT-2258\Beclanorsen\BP-100- |
| | | 1.02\G3139 + cytarabine (ARA-C)\G3139 + Dacarbazine\G3139 + |
| | | Dexamethasone\G3139 + Fludarabine\G3139 + G-CSF\G3139 + Irinotecan\G3139 + |
| | | Paclitaxel\G3139 + ThalidomidelVenetoclax (Venclexta)\Venetoclax + Bortezomib (BCL2 |
| | | inhibitor + Proteasome inhibitor)\BCL2 inhibitors\Eribulin liposomal\Paclitaxel\Paclitaxel |
| | | micelle formulation\Albumin-bound paclitaxel\Paclitaxel polymeric micelle |
| | | formulation\Proliposomal intravesical paclitaxel\Paclitaxel controlled-release\Paclitaxel |
| | | oral\Paclitaxel injectable suspension\Paclitaxel lipid nanocapsules\Paclitaxel |
| | | nanoformulation\Paclitaxel liposomal\IG 003\Paclitaxel polymer implant\Paclitaxel |
| | | nanosomal\Paclitaxel tocopheryl-based emulsion\TumorSelect Paclitaxel\Al |
| | | 850\Paclitaxel nanopolymer\Research programme: paclitaxel nanoparticles\Paclitaxel |
| | | nanoparticles\Research programme: sustained release paclitaxel\Paclitaxel |
| | | nanoparticle\Research programme: paclitaxel inhalation\Docetaxel liposomal\Docetaxel |
| | | polymeric micelle\Docetaxel plus ritonavir\Docetaxel\Docetaxel nanoparticle\Docetaxel |
| | | intratumoural\Docetaxel nanosomal\Docetaxel oral\Docetaxel emulsion\Docetaxel |
| | | injection\Docetaxel nanopolymer\Docetaxel alcohol-free injection |
| BRAF | Sorafenib tosylate | MLN2480\LY3009120\RO- |
| | (Nexavar); | 5126766\REGORAFENIB\Dabrafenib\LGX818\Vemurafenib\PLX8394\CEP-32496\ARQ |
| | Vemurafenib | 736\ASN003\BGB-283\BMS-908662\RO-5212054\RG-7256VAR-00341677\AZ- |
| | (Zelboraf); | 628\RG7304\PLX-4720\PLX-ORI3\XL |
| | Regorafenib | 281\trametinib\cobimetinib\binimetinib\encorafenib\Vemurafenib\Dabrafenib + |
| | (Stivarga); | Trametinib\Trametinib\Cobimetinib + Vemurafenib\Binimetinib + Encorafenib\ |
| | Dabrafenib | Cobimetinib\PLX8394\Dabrafenib (Tafinlar)\Regorafenib (Stivarga)\Sorafenib |
| | mesylate (Tafinlar); | (Nexavar)\Vemurafenib (Zelboraf)\Sorafenib\Selumetinib\PI3K Inhibitor GDC-0941 |
| | Encorafenib | Bismesylate\PLX4720\PLX4720\Nutlin-3\Capecitabine\Sorafenib\Pictilisib\MEK |
| | (Braftovi) | Inhibitor RO4987655\Refametinib\Dactolisib\Selumetinib\Pan-RAF Inhibitor |
| | | LY3009120\Gefitinib\Dabrafenib\GDC-0879\Mitogen-Activated Protein Kinase Inhibitor\ |
| | | MEK Inhibitor TAK-733\Temsirolimusllrinotecan\Oxaliplatin\Irinotecan\Regorafenib\ |
| | | Trametinib Dimethyl Sulfoxide\Trametinib Dimethyl Sulfoxidel U01261 |
| | | Encorafenib\Alpelisib\BRAF inhibitor + CDK2/4 inhibitors\BRAF inhibitor + HSP90 |
| | | inhibitors\BRAF inhibitor + MEK inhibitors\BRAF inhibitor + PI3K pathway inhibitors\ |
| | | BRAF inhibitors\ERK inhibitors\MEK inhibitors\Pan-RAF inhibitors\MEK inhibitors |
| | | (Trametinibletc)\Dabrafenib (BRAF inhibitor)\Dabrafenib + Trametinib (BRAF inhibitor + |
| | | MEK inhibitor)\Dasatinib (BCR-ABL inhibitor 2nd gen)\PLX4720 (BRAF inhibitor)\ |
| | | Selumetinib (MEK inhibitor)\Sorafenib (Pan-TK inhibitor)\Trametinib (MEK inhibitor)\ |
| | | Vemurafenib (BRAF inhibitor)\Vemurafenib + Cobimetinib (BRAF inhibitor + MEK |
| | | inhibitor)\Sorafenib nanoparticle formulation\RAF265\ARQ736\RO5212054\PLX3603 |
| BTK | Ibrutinib | Dasatinib\Acalabrutinib\Ibrutinib\Ibrutinib companion diagnostic\ACP-196\BGB- |
| | (Imbruvica); | 3111\BGB-3112\CC-292\GS-4059\ACP-319\Vecabrutinib\ARQ 531\BGB- |
| | Acalabrutinib | 3113\DTRMWXHS 12\GDC-0853\M7583\ONO-4059\Pyrrolo[2\3-d]pyrimidine derivative |
| | (Calquence); | 16\3-d]pyrimidine derivative 17\Fenebrutinib\Tirabrutinib\Spebrutinib\Ibrutinib (Imbruvica) |
| | Zanubrutinib | |
| | (Brukinsa) | |
| CDK4 | Palbociclib | LY2835219\Ribociclib Succinate\LEE011\BAY 10-00394\G1T38\P-276\P276-00\PD- |
| | (Ibrance); | 332991\Ro 31-7453\Trilaciclib\AG-024322\G1T28-1\P1446A-05\PHA-793887\RGB- |
| | Ribociclib succinate | 286638\R547\ZK 304709\INOC-005\CYC-103\PD- |
| | (Kisqali); | 0183812\Abemaciclib\Palbociclib\Apremilast\Palbociclib\Palbociclib (Ibrance)\Ribociclib |
| | Abemaciclib | (Kisqali)\Alpelisib\LEE011 (CDK4/6 inhibitor)\Ribociclib\Alvocidib\Nanoflavopiridol |
| | (Verzenio); | |
| | Trilaciclib 2HCl | |
| | (Cosela) | |
| CDK6 | Palbociclib | Ribociclib\Palbociclib\LY2835219\Ribociclib Succinate\LEE011\G1T38\PD- |
| | (Ibrance); | 332991\Trilaciclib\G1T28-1\NOC-005\CYC-103\PD-0183812\Palbociclib (Ibrance)\ |
| | Ribociclib succinate | Ribociclib (Kisqali)\Fulvestrant\Fulvestrant\CDK6 inhibitors\LEE011 (CDK 4/6 |
| | (Kisqali); | inhibitor)\Alvocidib\Nanoflavopiridol\Abemaciclib |
| | Abemaciclib | |
| | (Verzenio); | |
| | Trilaciclib 2HCl | |
| | (Cosela) | |
| CSF1R | Sunitinib malate | DASATINB\ARRY-382\FPA-008\BLZ-945\DCC-3014\PLX7486\SNDX- |
| | (Sutent); | 6352\Sulfatinib\JNJ-40346527\MCS-110\RG7155\AMG 820\IMC-CS4\Imatinib\GW- |
| | Pexidartinib HCl | 2580\Imatinib (BCR-ABL inhibitor 1st gen&KIT inhibitor)\Erdafitinib\Pexidartinib\Imatinib |
| | (Turalio) | sublingual |
| DNMT1 | Azacitidine (Vidaza) | Decitabine\CC-486\Guadecitabine\S-110\SGI110\Antroquinonol\Palifosfamide\RX- |
| | | 3117\Oral azacitidine\CP-4200\PMX-700\XB-0515-AZACYTIDINE\Cisplatin\Azacitidine |
| EGFR | Gefitinib (Iressa); | Dacomitinib\Neratinib\VANDETANIB\XL647\AFATINIB\AZD-9291 MESYLATE\BMS- |
| | Erlotinib HCl | 599626\BMS-690514\CUDC-101\PELITINIB\SAPITINIB\MUBRITINIB\S- |
| | (Tarceva); | 222611\LAPATINIB\ERLOTINIB |
| | Cetuximab | HYDROCHLORIDE\ICOTINIB\OLMUTINIB\SYM004\INDIUM-111\RO5083945\127 481- |
| | (Erbitux); | 29-2\AP26113\GELFITINIB\HUMAX-EGFR\RINDOPEPIMUTXL-999\MASITINIB\BIBW |
| | Panitumumab | 2992\Erlotinib\Gefitinib\NERATINIB MALEATE\Sorafenib\Tyverb/Tykerb\Icotinib |
| | (Vectibix); | hydrochloride\AZD9291\CO-1686\DE-766\EGF816\HKI-272\Varlitinib\ABT- |
| | Lapatinib Ditosylate | 414\ASP8273\CetuGEX\CI-1033\HER1-VSSP vaccine\HM-78136B\MEHD- |
| | (Tykerb); | 7945A\Pazopanib + Tyverb/Tykerb\SYM-004\TT-100\VATALANIB\CART-EGFR\EGFR |
| | Vandetanib | CART\EMD 55900\SN-32793\Sym013\Sym015\AMG 595\Anti-EGFR CAR T\Anti- |
| | (Caprelsa); Afatinib | HER3/EGFR DAF\AP32788\AST-1306\BIBX-1382\Cipatinib\EGFR806-specific CAR T |
| | dimaleate (Gilotrif); | cell\HER-2/HER-1 vaccine\IMGN289\JNJ-26483327\LY3164530\MM-151\MR1-1\RM- |
| | Osimertinib | 1929\Rogaratinib\SYN004\PKI166\RG7160\AZD4769\TAK 165\111In-hEGF\EGFR/IGFR |
| | mesylate | tandem adnectin\AZD-9935\CGP-52411\AFM-21\AGT-2000\AL-6802\ARX-AHD\EDP- |
| | (Tagrisso); | 13\Epitinib\GI-3000\HM-61713B\Imaging-theranostic nanoemulsion agents\IPS- |
| | Necitumumab | 01003\LA22-radioimmunoconjugates\MDP-01\MT-062\NRC-2694\ON-128\RX- |
| | (Portrazza); | 1792\SYM-011\Theliatinib\WZ-3146\CK-101\EGFR antisense |
| | Brigatinib | DNA\AC0010\Pyrotinib\Anti-EGFRvIII CAR transduced PBLVAnti-EGFR V III CAR-T |
| | (Alunbrig); | cells\EGFRvIII CARIAnti-EGFRvlll CAR T cells\CAR-T cells targeting EGFRviii\EGFRvIII |
| | Neratinib maleate | CAR T cells\EGFRvIII-CARs\CART-EGFRvIII T cells\Abivertinib\Avitinib\Nitroglycerin |
| | (Nerlynx); | transdermal\AZD 4769\BMS 690514\BMS 599626\Sorafenib nanoparticle |
| | Dacomitinib | formulation\Masoprocol\Research programme: intron fusion proteins\PD |
| | (Vizimpro); | 158780\Allitinib\CUDC 101\Canertinib\AG 1478\CGP 53353\PD |
| | Mobocertinib | 153035\regorafenib\osimertinib\Erlotinib\Gefitinib\Osimertinib\Afatinib\Dacomitinib\ |
| | succinate (Exkivity); | Poziotinib\Lapatinib\Afatinib (Gilotrif)\Aldesleukin (Proleukin)\Erlotinib (Tarceva)\ |
| | Amivantamab | Gefitinib (Iressa)\Lapatinib (Tykerb)\Osimertinib (Tagrisso)\Vandetanib (Caprelsa)\ |
| | (Rybrevant) | Pemetrexed\Staurosporine\ Multikinase Inhibitor AEE788\Rociletinib\ Cisplatin\ |
| | | Epidermal Growth Factor Receptor Tyrosine Kinase Inhibitor\Docetaxel\Rociletinib\ |
| | | Rindopepimut\Sirolimus\Neratinib\Canertinib\Multikinase Inhibitor AEE788\Crizotinib\ |
| | | Cytoreductive Surgery\Hyperthermic Intraperitoneal Chemotherapy\EGFR inhibitor 3rd |
| | | gensl EGFR inhibitors\EGFR TK inhibitors\HSP90 inhibitors\MEK inhibitor (alone or in |
| | | combination)s\EGFR inhibitor 3rd gens (Rociletinib\HM61713\etc)\Afatinib (ERBB2 |
| | | inhibitor&EGFR inhibitor 2nd gen)\Erlotinib (EGFR inhibitor 1st gen)\Gefitinib (EGFR |
| | | inhibitor 1st gen)\Lapatinib (ERBB2 inhibitor)\Osimertinib (EGFR inhibitor 3rd gen)\ |
| | | Rindopepimut (Vaccine)\TSU-68\Alvocidib\Nanoflavopiridol |
| ERBB2 | Trastuzumab | VANDETANIB\XL647\AFATINIB\AZD-9291 MESYLATE\DACOMITINIB\BMS- |
| | (Herceptin); | 599626\BMS-690514\CUDC-101\PELITINIB\SAPITINIB\MUBRITINIB\S- |
| | Lapatinib | 222611\LAPATINIB\ERLOTINIB |
| | Ditosylate | HYDROCHLORIDE\ICOTINIB\OLMUTINIB\SYM004\INDIUM-111\RO5083945\127 481- |
| | (Tykerb); | 29-2\AP26113\GELFITINIB\HUMAX-EGFR\RINDOPEPIMUT\BIBW |
| | Pertuzumab | 2992\Masoprocol\NERATINIB MALEATE\Tyverb/Tykerb\HKI-272\Nelipepimut |
| | (Perjeta); Afatinib | S\NeuVax\PF-05280014\Taxol/Paraplatin/Herceptin\Varlitinib\Anti-CD3 and anti- |
| | dimaleate (Gilotrif); | Her2/neu bispecific antibody-armed activated T cells\AZD8931\CI-1033\CP- |
| | Ado-trastuzumab | 724714\DN24-02\HER-2 Protein AutoVac\HER2/neu peptide vaccine\Her2-targeted |
| | emtansine | autologous T-cell therapy\HM-78136B\MCLA-128\MGAH22\MM-111\Pazopanib + |
| | (Kadcyla); | Tyverb/Tykerb\ABY-025\Anti-HER2 CAR-T\AU105\AVX901\CART-HER-2\HER-2- |
| | Neratinib maleate | targeting CAR T Cells\Sym013\ARRY-380\CAR-T cells targeting HER2\Cipatinib\HER2- |
| | (Nerlynx); | CAR T Cells\HER2p63-71 peptide vaccine\HER2-specific CAR T cell\HER2-specific T |
| | Trastuzumab | cells\JNJ-26483327\MEDI4276\MM-302\MVA HER-2 Auto Vac\Recombinant human |
| | deruxtecan | Erbb3 fragment therapeutic tumor vaccine\TAK-285\TrasGEXIVM-206\Her-2-Bi-armed |
| | (Enhertu); | ATCITAK 165\GW-974\Tucatinib\PNT-500\CP |
| | Tucatinib (Tukysa); | 724714\everolimus\fulvestrantlolaparib\\xabepilone\neratinib\palbociclib\ribociclib\abema |
| | Margetuximab | ciclibleribulin\talazoparib\Lapatinib\Neratinib\Afatinib (Gilotrif)\Aldesleukin (Proleukin)\ |
| | (Margenza) | Lapatinib (Tykerb)\Neratinib (Nerlynx)\AKTi-1/2\Afatinib\Sirolimus\Irinotecan\ |
| | | Dacomitinib\Docetaxel\Capecitabine\Capecitabinel Pictilisib\A66\Erlotinib\Gefitinib\ |
| | | Pilaralisib\Palbociclib\MTOR Kinase Inhibitor PP242\Platinum |
| | | Compound\Paclitaxel\Zoledronic Acid\ERBB2 inhibitor + CDK4/6 inhibitors\ERBB2 |
| | | inhibitors (Afatinibletc)\Afatinib (ERBB2&EGFR inhibitor 2nd gen)\Dacomitinib (Pan |
| | | ERBB inhibitor)\Lapatinib (ERBB2 inhibitor)\Lapatinib + Chemotherapy (ERBB2 |
| | | inhibitor + Chemotherapy)\Neratinib (ERBB2 inhibitor)\Tensirolimus (MTOR inhibitor) |
| ERBB4 | Afatinib dimaleate | VANDETANIB\XL647\AFATINIB\AZD-9291 MESYLATE\DACOMITINIB\BMS- |
| | (Gilotrif); Neratinib | 599626\BMS-690514\CUDC-101\PELITINIB\SAPITINIB\MUBRITINB\S- |
| | maleate (Nerlynx) | 222611\LAPATINIB\CI-1033\JI-101\RG7388\JNJ-26483327\Lapatinib (ERBB2 inhibitor)\ |
| | | Lapatinib + Afatinib (ERBB2 inhibitor + ERBB2 inhibitor&EGFR inhibitor 2nd gen) |
| ESR1 | Toremifene citrate | Mitotane\LAPATINIB\XL-999\FULVESTRANT\Fulvestrant intraductal\Tamoxifen |
| | (Fareston); | liposomal\Tamoxifen oral liquid |
| | Fulvestrant | |
| | (Faslodex); | |
| | Ospemifene | |
| | (Osphena) | |
| EZH2 | Tazemetostat | EPZ-6438\CPI-1205\DS-3201\GSK2816126\Tazemetostat\GSK 126\EZH2 inhibitors |
| | (Tazverik) | (EPZ-005687\etc) |
| FGFR1 | Pazopanib HCl | Ponatinib\Sorafenib nanoparticle formulation\Dovitinib\Erdafitinib\Regorafenib\XL- |
| | (Votrient); | 999\MASITINIB\TSU-68\NVP-BGJ398\PALIFERMIN\SU5402\Intedanib\Romiplostim\E- |
| | Regorafenib | 3810\Rosiglitazone + metformin\AZD4547\ARQ-087\BGJ398\FP- |
| | (Stivarga); | 1039\NCB54828\Debio 1347\JNJ-42756493\KW-2449\Sulfatinib\BMS-695735\PD- |
| | Nintedanib esylate | 0183812\ACTB-1003\Nintedanib\AZD 4547\pazopanib\BGJ398\Debio 1347\ |
| | (Ofev); Lenvatinib | Erdafitinib\Ponatinib (Iclusig)\PD173074\Ponatinib\Infigratinib\Dovitinib\Midostaurin\ |
| | mesylate (Lenvima); | Afimoxifenel FGFR Inhibitor AZD4547\Infigratinib\FGF/VEGF Receptor Tyrosine Kinase |
| | Erdafitinib | Inhibitor\PD173074\FGFR inhibitors\FGFR inhibitors (Lucitanibletc)\BRIVANIB |
| | (Balversa); | ALANINATE\TRAFERMIN\Lenvatinib |
| | Pemigatinib | |
| | (Pemazyre); | |
| | Infigratinib | |
| | phosphate | |
| | (Truseltiq) | |
| FGFR2 | Pazopanib HCl | Ponatinib\Dovitinib\Erdafitinib\Regorafenib\LAPATINIBIXL-999\MASITINIBITSU- |
| | (Votrient); | 68\THALIDOMIDE\NVP-BGJ398\PALIFERMIN\BRIVANIB ALANINATE\TRAFERMINIE- |
| | Regorafenib | 3810\NCB54828\LY2874455\AEE-788\Debio 1347\BAY1179470\PD-0183812\ACTB- |
| | (Stivarga); | 1003\AEE 788\RP 52028\AZD4547\BGJ398\Debio 1347\Erdafitinib\Ponatinib (Iclusig)\ |
| | Nintedanib esylate | Pazopanib\PD173074\Dovitinib\Infigratinib\Infigratinib\PD173074\FGFR Inhibitor |
| | (Ofev); Lenvatinib | AZD4547\FGFR inhibitors\Ponatinib (BCR-ABL inhibitor 3rd gen&Pan-TK |
| | mesylate (Lenvima); | inhibitor)\Lenvatinib\Nintedanib |
| | Erdafitinib | |
| | (Balversa); | |
| | Pemigatinib | |
| | (Pemazyre); | |
| | Infigratinib | |
| | phosphate | |
| | (Truseltiq) | |
| FGFR3 | Nintedanib esylate | Ponatinib\Dovitinib\Erdafitinib\XL-999\MASITINIBITSU-68\NVP- |
| | (Ofev); Lenvatinib | BGJ398\PALIFERMINISU5402\BRIVANIB ALANINATE\TRAFERMIN\Trapidil\BMS- |
| | mesylate (Lenvima); | 582664\E-3810\B-701\NCB54828\AEE-788\Debio 1347\TKI258\Anti-FGFR3\PD- |
| | Erdafitinib | 0183812\ACTB-1003\AV-370\pazopanib\AZD4547\BGJ398\Debio 1347\ |
| | (Balversa); | Erdafitinib\Ponatinib (Iclusig)\PD173074\Tyrosine Kinase Inhibitor SU5402\Pazopanib\ |
| | Pemigatinib | Infigratinib\FGFR inhibitors\Bortezomib (Proteasome inhibitor)\FGFR inhibitors |
| | (Pemazyre); | (letc)\Lenvatinib\Nintedanib |
| | Infigratinib | |
| | phosphate | |
| | (Truseltiq) | |
| FGFR4 | Nintedanib esylate | Ponatinib\Erdafitinib\XL-999\TSU-68\NVP-BGJ398\PALIFERMIN\BRIVANIB |
| | (Ofev); Lenvatinib | ALANINATE\TRAFERMIN\FGF401\BLU-554\H3B-6527\PD-0183812\INCB62079\ABC- |
| | mesylate (Lenvima); | 4\ACTB-1003\Fisogatinib\Roblitinib\FGFR inhibitors\Lenvatinib |
| | Erdafitinib | |
| | (Balversa) | |
| FLT1 | Sunitinib malate | Sorafenib nanoparticle |
| | (Sutent); | formulation\Regorafenib\Axitinib\Lenvatinib\Nintedanib\SUNITINIB\LINIFANIB\XL- |
| | Pazopanib HCl | 999\ENMD- |
| | (Votrient); Axitinib | 2076\SORAFENIB\CEDIRANIB\FORETINIB\TIVOZANIB\MOTESANIB\VATALANIB\MG |
| | (Inlyta); | CD-265\TELATINIB\OSI-930\X-82\PAZOPANIB |
| | Regorafenib | |
| | (Stivarga); | |
| | Cabozantinib S- | |
| | malate (Cometriq); | |
| | Nintedanib esylate | |
| | (Ofev); Lenvatinib | |
| | mesylate (Lenvima); | |
| | Tivozanib HCl | |
| | (Fotivda) | |
| FLT3 | Sorafenib tosylate | Fedratinib\Ponatinib\Gilteritinib\Sorafenib nanoparticle |
| | (Nexavar); | formulation\Pexidartinib\Nintedanib\SUNITINIB\LINIFANIB\XL-999\ENMD- |
| | Sunitinib malate | 2076\SORAFENIB\CEDIRANIB\FORETINIB\Intedanib\Lestaurtinib\Midostaurin\AC220\P |
| | (Sutent); | acritinib\PLX-3397\CDX-301\Famitinib\TAK-659\SEL-24\4SC-203\FF-10101-01\IMC- |
| | Cabozantinib S- | EB10\Ki23819\KW-2449\Tandutinib\MC-2001\AKN-028\Quizartinib\venetoclax\High |
| | malate (Cometriq); | Dose Chemotherapy + Midostaurin\Gilteritinib\Sorafenib\Crenolanib\ |
| | Ponatinib HCl | Quizartinib\Cabozantinib (Cabometyx [tablet]\Cometriq [capsule])\Midostaurin (Rydapt)\ |
| | (Iclusig); | Ponatinib (Iclusig)\Tretinoin\Daunorubicin\SU5614\Lestaurtinib\Tyrphostin AG 1296\ |
| | Nintedanib esylate | Ponatinib\Anthracycline Antineoplastic Antibiotic\Selumetinib\Midostaurin\Sunitinib\ |
| | (Ofev); Brigatinib | Tandutinib\Cytarabine\Daunorubicin\AS602868\Tyrphostin AG 1296\FLT3 inhibitors\ |
| | (Alunbrig); | novel FLT3 inhibitors\Crenolanib (FLT3 inhibitor)\Lestaurtinib (Pan-TK inhibitor)\ |
| | Midostaurin | Midostaurin (Pan-TK inhibitor)\Quizartinib (Pan-TK inhibitor)\Sorafenib (Pan-TK |
| | (Rydapt); | inhibitor)\Sorafenib + Azacytidine (Pan-TK inhibitor + |
| | Gilteritinib fumarate | Chemotherapy)\CABOZANTINIB\AMUVATINIB\PLX108-01 |
| | (Xospata); | |
| | Pexidartinib HCl | |
| | (Turalio); Fedratinib | |
| | HCl (Inrebic) | |
| FLT4 | Sorafenib tosylate | Sorafenib nanoparticle |
| | (Nexavar); | formulation\Regorafenib\Axitinib\Lenvatinib\Nintedanib\SUNITINIB\LINIFANIB\XL- |
| | Sunitinib malate | 999\ENMD- |
| | (Sutent); | 2076\SORAFENIB\CEDIRANIB\FORETINIB\TIVOZANIB\MOTESANIB\VATALANIB\MG |
| | Pazopanib HCl | CD-265\TELATINIB\OSI-930\X-82\PAZOPANIB\ABT-869\E-3810\Fruquintinib\PI- |
| | (Votrient); | 88\Famitinib\IMC-3C5\JNJ-26483327\Sulfatinib\VEGFR3 Monoclonal Antibody\MG- |
| | Vandetanib | 516\SAR-131675\cabozantinib |
| | (Caprelsa); Axitinib | |
| | (Inlyta); | |
| | Regorafenib | |
| | (Stivarga); | |
| | Cabozantinib S- | |
| | malate (Cometriq); | |
| | Nintedanib esylate | |
| | (Ofev); Lenvatinib | |
| | mesylate (Lenvima); | |
| | Tivozanib HCl | |
| | (Fotivda) | |
| HDAC1 | Vorinostat (Zolinza); | Panobinostat\Romidepsin\GIVINOSTAT\STAUROSPORINE\INDIRUBIN-3′- |
| | Romidepsin | MONOXIME\Vorinostat\HBI-8000\ITF2357\NVP-LAQ824\MGCD- |
| | (Istodax); | 0103\Resminostat\SNDX-275\Sodium butyrate\CHR-3996\OBP-801\SB-639\AN- |
| | Belinostat | 9\Tacedinaline\Pyroxamide\4SC-202\Chlamydocin\Depudecin\HC-Toxin\M- |
| | (Beleodaq); | carboxycinnamic acid bishydroxamide\Scriptaid\SK-7041\SK-7068\Oxamflatin\ST- |
| | Panobinostat | 2987\CBHA\Abexinostat\Belinostat (Beleodaq)\Panobinostat (Farydak)\Romidepsin |
| | lactate (Farydak) | (Istodax)\Vorinostat |
| | | (Zolinza)\Mocetinostat\BELINOSTAT\ENTINOSTAT\PRACINOSTAT\935881-37- |
| | | 1\DACINOSTAT\TRICHOSTATIN A |
| HDAC2 | Vorinostat (Zolinza); | Panobinostat\Romidepsin\GIVINOSTAT\Vorinostat\Mocetinostat\BELINOSTAT\ENTINO |
| | Romidepsin | STAT\PRACINOSTAT\935881-37-1\DACINOSTAT\SCRIPTAID\TRICHOSTATIN |
| | (Istodax); | AICHR-3996\ST-2987\Belinostat (Beleodaq)\Panobinostat (Farydak)\Romidepsin |
| | Belinostat | (Istodax)\Vorinostat (Zolinza)\Olaparib (PARP inhibitor) |
| | (Beleodaq); | |
| | Panobinostat | |
| | lactate (Farydak) | |
| HDAC3 | Vorinostat (Zolinza); | Panobinostat\GIVINOSTAT\Vorinostat\Mocetinostat\ROMIDEPSIN\BELINOSTAT\ENTIN |
| | Romidepsin | OSTAT\PRACINOSTAT\935881-37-1\DACINOSTAT\SCRIPTAID\TRICHOSTATIN |
| | (Istodax); | AICHR-3996\Belinostat (Beleodaq)\Panobinostat (Farydak)\Romidepsin (Istodax)\ |
| | Belinostat | Vorinostat (Zolinza) |
| | (Beleodaq); | |
| | Panobinostat | |
| | lactate (Farydak) | |
| IDH1 | Ivosidenib (Tibsovo) | Ivosidenib\AG-120\DH305\BAY1436032\IDH 305\venetoclax\GSK321\AGI-51981 |
| | | Temozolomide\Ivosidenib\BPTES\Olaparib\BCL2 inhibitors\IDH1 inhibitors\PARP |
| | | inhibitors\AG-120 (IDH1 inhibitor)\Dasatinib (BCR-ABL inhibitor 2nd gen)\Venetoclax |
| | | (BCL2 inhibitor) |
| IDH2 | Enasidenib | Enasidenib\ENASIDENIB MESYLATE\AG-221\AG-881\venetoclax\Enasidenib |
| | mesylate (Idhifa) | (Idhifa)\AG-221 (IDH2 inhibitor)\Dasatinib (BCR-ABL inhibitor 2nd gen)\Venetoclax |
| | | (BCL2 inhibitor) |
| IGF1R | Ceritinib (Zykadia); | AMG 479\AVE-1642\MECASERMIN\RENZAPRIDE\BIIB022\NVP- |
| | Brigatinib | AEW541\LINSITINIB\OSI-906\Rinfabate\AXL-1717\MM-141\TT-100\AEW-541\BIIB |
| | (Alunbrig); | 022\Cyclolignan picropodophyllin\KW-2450\BMS-695735\EGFR/IGFR tandem |
| | Teprotumumab | adnectin\JB-1\NVP-ADW742\Gefitinib\IGF1R Monoclonal Antibody |
| | (Tepezza) | |
| JAK1 | Ruxolitinib | Fedratinib\Ruxolitinib\Cerdulatinib\INCB039110\Ruxolitinib (Jakafi)\Enzastaurin\ |
| | phosphate (Jakafi); | Ruxolitinib\Ruxolitinib (JAK inhibitor)\TOFACITINIB\TOFACITINIB CITRATEIXL019 |
| | Tofacitinib citrate | |
| | (Xeljanz); | |
| | Baricitinib | |
| | (Olumiant); | |
| | Upadacitinib | |
| | (Rinvog) | |
| JAK2 | Ruxolitinib | Entrectinib\Ruxolitinib\TOFACITINIB\TOFACITINIB |
| | phosphate (Jakafi); | CITRATE\XL019\TF2357\Momelotinib\Pacritinib\TG101348\AZD1480\BMS- |
| | Tofacitinib citrate | 911543\Cerdulatinib\INCB039110\LY2784544\NS-018\AC430\SB-1578\AG490\AMG- |
| | (Xeljanz); | JAK2-01\BVB-808\Jak2 inhibitors\Jak2 tyrosine inhibitors\K-454\ON-044580\SGI- |
| | Baricitinib | 1252\Fedratinib\Givinostat\Peginterferon beta\Research programme: Janus kinase 2 |
| | (Olumiant); | inhibitors\AG 490\Ruxolitinib (Jakafi)\Ruxolitinib\JAK inhibitor (alone or in combination)s\ |
| | Upadacitinib | JAK inhibitors\Ruxolitinib (JAK inhibitor)\AT9283\ENMD-2076 |
| | (Rinvoq); | |
| | Fedratinib HCl | |
| | (Inrebic) | |
| JAK3 | Tofacitinib citrate | RUXOLITINIB\TOFACITINIB\TOFACITINIB |
| | (Xeljanz); | CITRATE\XL019\AT9283\LY2784544\Cerdulatinib\AG490\Tofacitinib (Xeljanz)\JAK |
| | Baricitinib | inhibitors |
| | (Olumiant); | |
| | Upadacitinib | |
| | (Rinvoq) | |
| KDR | Sorafenib tosylate | Ponatinib\Sorafenib nanoparticle |
| | (Nexavar); | formulation\Erdafitinib\Cabozantinib\Regorafenib\Axitinib\Lenvatinib\Nintedanib\Foretinib\ |
| | Sunitinib malate | ENMD-2076\Pazopanib HCl\Romiplostim\Sorafenib\Sunitinib\Vandetanib\YN- |
| | (Sutent); | 968D1\Brivanib\Cediranib\E-3810\Fruquintinib\HKI-272\Rosiglitazone + |
| | Pazopanib HCl | metformin\Tivozanib\BAY-57-9352\CP- |
| | (Votrient); | 547632\Famitinib\RAF265\VATALANIB\XL880\Anti-VEGFR2 CD8 cell |
| | Vandetanib | therapy\Elpamotide\OTSGC-A24\Altiratinib\CEP-11981\CYC116\E-7050\KRN633\OSI- |
| | (Caprelsa); Axitinib | 930\Pegdinetanib\PF-00337210\Rivoceranib\Sulfatinib\TAK-593\XL999\Antibodie |
| | (Inlyta); | derivative 10\Motesanib\SU-14813\MC-1C11\CEP-5214\AMG-429\EPI-0030\MG- |
| | Regorafenib | 516\PLX-4720\R-84\TTAC-0001\SU 14813\CYC 116\CEP 11981\497839-62-0\BMS- |
| | (Stivarga); | 690514\MGCD-265\TSU-68\XL647\252003-65-9\CYC-116\X-82\UNII- |
| | Cabozantinib S- | RFG3GJ6251\867331-82-6\CEP-7055\KRN-633\pazopanib\midostaurin\Lenvatinib |
| | malate (Cometriq); | (Lenvima)\Sunitinib\Regorafenib\VEGFR inhibitors\XL-999\452105-23- |
| | Nintedanib esylate | 6\DASATINIB\PAZOPANIB HYDROCHLORIDE\SUNITINIB |
| | (Ofev); | MALATE\MASITINIB\DOVITINIB\LINIFANIB\PEXIDARTINIB\TELATINIB\XL-820 |
| | Ramucirumab | |
| | (Cyramza); | |
| | Lenvatinib mesylate | |
| | (Lenvima); | |
| | Tivozanib HCl | |
| | (Fotivda) | |
| KIT | Sorafenib tosylate | Dasatinib\Ponatinib\Nilotinib\Nilotinib nanoparticle formulation\Sorafenib nanoparticle |
| | (Nexavar); | formulation\Erdafitinib\Pexidartinib\Regorafenib\Lenvatinib\ENMD-2076\FORETINIB\XL- |
| | Sunitinib malate | 999\452105-23- |
| | (Sutent); | 6\SORAFENIB\SUNITINIB\AXITINIB\MIDOSTAURIN\CABOZANTINIB\PAZOPANIB\ |
| | Pazopanib HCl | PAZOPANIB HYDROCHLORIDE\SUNITINIB |
| | (Votrient); | MALATE\CEDIRANIB\MASITINIB\MOTESANIB\DOVITINIB\LINIFANIB\VATALANIB\ |
| | Regorafenib | TELATINIB\XL-820\Imatinib\Pazopanib HCl\Romiplostim\STI571\DCC-2618\PLX- |
| | (Stivarga); | 3397\Famitinib\MP470\BLU-285\CART-117 cells\OSI-930\PLX9486\CAR-T cells |
| | Cabozantinib S- | targeting CD117\APCK-110\WBZ-7\Imatinib sublingual\Research programme: FLT-3 |
| | malate (Cometriq); | tyrosine kinase inhibitors\Regorafenib\Imatinib\Sorafenib\Avapritinib\Axitinib (Inlyta)\ |
| | Ponatinib HCl | Cabozantinib (Cabometyx [tablet]\Cometriq [capsule])\Imatinib (Gleevec)\Pazopanib |
| | (Iclusig); Lenvatinib | (Votrient)\Regorafenib (Stivarga)\Sorafenib (Nexavar)\Dasatinib\Sunitinib\Pictilisibl |
| | mesylate (Lenvima); | Selumetinib\Midostaurin\Tandutinib\PD-180970\Ponatinib\Imatinib Mesylate\HSP90 |
| | Pexidartinib HCl | inhibitors\Dasatinib (BCR-ABL inhibitor 2nd gen)\Imatinib (BCR-ABL inhibitor 1st |
| | (Turalio); | gen&KIT inhibitor)\Nilotinib (BCR-ABL inhibitor 2nd gen)\Ponatinib (BCR-ABL inhibitor |
| | Avapritinib | 3rd gen&Pan-TK inhibitor)\Regorafenib (Pan-kinase inhibitor)\Sorafenib (Pan-TK |
| | (Ayvakit); Ripretinib | inhibitor)\Sunitinib (Pan-TK inhibitor)\Amuvatinib\TANDUTINIB\PLX108-01 |
| | (Qinlock) | |
| KRAS | Sotorasib | AZD4785\GI-4000 in combination with gemcitabine\PNT- |
| | (Lumakras) | 300\vemurafenib\dabrafenib\regorafenib\trametinib\Regorafenib\Cobimetinib\ |
| | | Binimetinib\Trametinib\Gefitinib\Erlotinib\Dactolisib\Selumetinibl Gemcitabine\ |
| | | Carboplatin\Paclitaxel\Abemaciclib\Palbociclib\Decitabine\Cisplatin\Akt Inhibitor |
| | | MK2206\Epidermal Growth Factor Receptor Tyrosine Kinase |
| | | Inhibitor\Immunomodulatory Oligonucleotide\Dasatinib\Selumetinib\B-Raf/VEGFR-2 |
| | | Inhibitor RAF265\B-Raf/VEGFR-2 Inhibitor RAF265\Afatinib\Docetaxel\PI3Kbeta |
| | | Inhibitor AZD8186\SCH772984\Chemotherapy\G-573\MEK Inhibitor GDC-06231 |
| | | Docetaxel\ARS-853\Epidermal Growth Factor Receptor Tyrosine Kinase Inhibitor\ |
| | | Ixazomib\AZD5438\Irinotecan\Carboplatin\Sorafenib\Vemurafenib\Refametinib\ |
| | | Therapeutic Tumor Infiltrating Lymphocytes\Dactolisib\Gefitinib\Melphalan\FOLFOX-4 |
| | | Regimen\Erlotinib\Pemetrexed\Binimetinib\BET inhibitors\CDK4 inhibitors\CDK 4/6 |
| | | inhibitor + MEK inhibitors\ERK inhibitors\FAK inhibitors\FAS inhibitors\HSP90 inhibitor |
| | | (in combination)s\JAK/TBK 1/IKKOEμ inhibitors\MEK inhibitor + BCL-XL inhibitors\MEK |
| | | inhibitor + IGF1R inhibitors\MEK inhibitor + PI3K pathway inhibitors\MEK inhibitors\ |
| | | MTOR inhibitor + BH3 mimeticss\pan-RAF inhibitors\PI3K pathway inhibitor + MEK |
| | | inhibitors\Abemaciclib (CDK4/6 inhibitor)\Decitabine + BCL2 inhibitor (Chemotherapy + |
| | | BCL2 inhibitor)\Gemcitabine + MEK inhibitor (Chemotherapy + MEK inhibitor)\ |
| | | Selumetinib (MEK inhibitor)\Sorafenib + MEK inhibitor (Pan-TK inhibitor + MEK |
| | | inhibitor)\Trametinib + Ponatinib (MEK inhibitor + BCR-ABL inhibitor 3rd gen&Pan-TK |
| | | inhibitor) |
| MAP2K1 | Trametinib dimethyl | Bosutinib\Binimetinib\E 6201\AZD 8330\Cobimetinib\Pimasertib\RG |
| | sulfoxide (Mekinist); | 7167\ESTRAMUSTINE\SELUMETINIB\AZD8330\TRAMETINIB\391210-10- |
| | Selumetinib sulfate | 9\AZD6244\RDEA-436\Mirdametinib\Trametinib\Cobimetinib (Cotellic)\Trametinib |
| | (Koselugo) | (Mekinist)\Vemurafenib\ERK inhibitors\novel MEK inhibitors\Selumetinib (MEK |
| | | inhibitor)\Trametinib (MEK inhibitor)\REFAMETINIB\212631-79-3\TAK- |
| | | 733\RO4987655\BAY869766 |
| MAP2K2 | Trametinib dimethyl | Bosutinib\Binimetinib\E 6201\AZD 8330\Cobimetinib\PimasertibIRG |
| | sulfoxide (Mekinist); | 7167\SELUMETINIB\AZD8330\TRAMETINIB\391210-10-9\REFAMETINIB\212631-79- |
| | Selumetinib sulfate | 3\TAK-733\RO4987655\BAY869766\AZD6244\RDEA-436\Cobimetinib (Cotellic)\ |
| | (Koselugo) | Trametinib (Mekinist) |
| MET | Crizotinib (Xalkori); | Cabozantinib\Crizotinib\Amuvatinib\BMS-777607\MK- |
| | Cabozantinib S- | 2461\RG3638\Savolitinib\Tivantinib\AMG 208\AMG 337\EMD-1214063\HM- |
| | malate (Cometriq); | 5016504\NCB28060\LY2801653\LY-2875358\SAR-125844\Volitinib\XL880\Anti-C-met |
| | Capmatinib HCl | CAR-T cells\MGCD516\MSC2156119\Sym015\ABBV-399\ABT-700\Altiratinib\CBT- |
| | (Tabrecta); | 101\E-7050\EMD-1204831\JNJ-38877605\LY3164530\Merestinib\LY2801653\MK- |
| | Tepotinib HCl | 8033\PF-4217903\SGX523\Autologous T Cells Expressing MET scFv CAR\C-Met/PD-L1 |
| | (Tepmetko); | CAR-T Cell\ALD-805\ALD-806\APS-3010\BAY-85-3474\CMET Avimer polypeptides\C- |
| | Amivantamab | Met kinase inhibitors\DP-3590\HuMax-cMet\LA-480\MG-516\PF-00614435\PHA- |
| | (Rybrevant) | 665752\PRS-110\RP-1040\TP-801\Capmatinib\BMS 777607\PF 4217903\SGX- |
| | | 523\AMG-208\PF-04217903\INCB028060\LY2875358\Cabozantinibl Capmatinibl |
| | | Erlotinib\Gefitinib\Osimertinib\Crizotinib\Cabozantinib (Cabometyx [tablet]\Cometriq |
| | | [capsule])\Crizotinib (Xalkori)\Foretinib\Gefitinib\Vemurafenib\Vemurafenib\Savolitinibl |
| | | Anti-EGFR Monoclonal Antibody\Trametinib\Selumetinib\Tepotinib\Savolitinib (MET |
| | | inhibitor)\BCL2 inhibitors\MET inhibitors\MET inhibitors (Crizotinibletc)\Crizotinib (ALK |
| | | inhibitor)\FORETINIB\MGCD-265 |
| MTOR | Sirolimus | Sirolimus\Sirolimus albumin bound Encapsulated sirolimus\Sirolimus polymeric |
| | (Rapamune); | nanoparticle\Everolimus\PF- |
| | Temsirolimus | 04449913\Rapamycin\Temsirolimus\Zotarolimus\Ridaforolimus\ABI- |
| | (Torisel); | 009\AZD2014\BEZ235\GDC-0980/RG7422\INK128\LY3023414\MM-141\OSI-027\PF- |
| | Everolimus (Afinitor) | 04691502\PF-05212384\Salirasib\SAR245409\SF1126\BGT226\CC-223\ME-344\BI |
| | | 860585\DS-3078\DS-7423\GDC-0349\PWT-33597\VE-822\VS-5584\AZD8055\SCR- |
| | | 44001\AR-mTOR-26\CU-906\Dual PI3K/mTOR inhibitors\Dual PI3K-alpha/mTOR |
| | | inhibitors\EC-0565\EM-101\HM-5016699\MTOR inhibitors (cancer)\Amgen\OXA-01\P- |
| | | 6915\PF-03772304\PF-05094037\SB-2280\SX-MTR1\X- |
| | | 387\Dactolisib\Sapanisertib\Bimiralisib\Vistusertib\Apitolisib\AZD 8055\BGT 226\SF |
| | | 1126\FTS\DEFOROLIMUS\1224844-38-5\DS-3078A\PIMECROLIMUS\Everolimus\ |
| | | Temsirolimus\Everolimus (Afinitor)\Temsirolimus (Torisel)\Pazopanib\ |
| | | Capivasertib\PI3K/BET Inhibitor LY294002\Sirolimus\Pazopanibl MTOR Kinase |
| | | Inhibitor AZD8055\MTOR kinase inhibitors\Everolimus (MTOR inhibitor)\Sirolimus |
| | | (MTOR inhibitor)\Gedatolisib\PF 4691502 |
| PARP1 | Olaparib | OLAPARIB\Rucaparib\KU-0058948\Niraparib Tosylate\CC-486\AG140699\CPH-102\A- |
| | (Lynparza); | 620223\ANG-2864\BPI-704001\INO-1002\S-070\S-111\NicotinamidelResearch |
| | Rucaparib | programme: PARP inhibitors\NU 1025\PD 128763\Iniparib\4-IODO-3- |
| | camsylate | NITROBENZAMIDE\Niraparib (Zejula)\Olaparib (Lynparza)\Rucaparib |
| | (Rubraca); | (Rubraca)\Veliparib\Talazoparib\Niraparib\BMN673 |
| | Niraparib tosylate | |
| | (Zejula); | |
| | Talazoparib tosylate | |
| | (Talzenna) | |
| PARP2 | Olaparib | OLAPARIB\Rucaparib\Veliparib\Talazoparib\Niraparib\BMN673\Niraparib (Zejula)\ |
| | (Lynparza); | Olaparib (Lynparza)\Rucaparib (Rubraca) |
| | Rucaparib | |
| | camsylate | |
| | (Rubraca); | |
| | Niraparib tosylate | |
| | (Zejula); | |
| | Talazoparib tosylate | |
| | (Talzenna) | |
| PARP3 | Olaparib | OLAPARIB\Rucaparib\VELIPARIB\Niraparib (Zejula)\Olaparib (Lynparza)\Rucaparib |
| | (Lynparza); | (Rubraca) |
| | Rucaparib | |
| | camsylate | |
| | (Rubraca); | |
| | Niraparib tosylate | |
| | (Zejula); | |
| | Talazoparib tosylate | |
| | (Talzenna) | |
| PDGFRA | Regorafenib | Ponatinib\Erdafitinib\Regorafenib\Lenvatinib\Nintedanib\Amuvatinib\SUNITINIB\MEDI- |
| | (Stivarga); | 575\IMATINIB\PAZOPANIB\CRENOLANIB\DOVITINIB\MASITINIB\MOTESANIB\XL- |
| | Nintedanib esylate | 820\TANDUTINIB\X-82\TAK-593\BECAPLERMIN\MIDOSTAURIN\ALPELISIB\PKI- |
| | (Ofev); Lenvatinib | 587\PF-04691502\APITOLISIB\XL765\SF1126\COPANLISIB\944396-07-0\GDC- |
| | mesylate (Lenvima); | 0941\PX-866\GSK2636771\XL147\AXITINIB\Romiplostim\CP-868596\DCC-2618\E- |
| | Olaratumab | 3810\Famitinib\MP470\BLU-285\PMID28460551-Compound-1\SRI-62-834\CEP- |
| | (Lartruvo); | 2563\Ripretinib\Avapritinib\AG 1295\Dasatinib\Pazopanib (Votrient)\Sorafenib |
| | Avapritinib | (Nexavar)\Crenolanibl Sunitinib\Ponatinib\Imatinib\Imatinib Mesylate\ |
| | (Ayvakit); Ripretinib | Sorafenib\HSP90 inhibitors\Crenolanib (FLT3 inhibitor)\Dasatinib (BCR-ABL inhibitor |
| | (Qinlock) | 2nd gen)\Imatinib (BCR-ABL inhibitor 1st gen&KIT inhibitor)\Regorafenib (Pan-kinase |
| | | inhibitor)\Sorafenib (Pan-TK inhibitor)\Sunitinib (Pan-TK |
| | | inhibitor)\LINIFANIB\TELATINIB\VATALANIB\CEDIRANIB\TSU-68\XL- |
| | | 999\DASATINIB\Imatinib sublingual |
| PDGFRB | Sorafenib tosylate | Dasatinib\Sorafenib nanoparticle |
| | (Nexavar); | formulation\Erdafitinib\Pexidartinib\Regorafenib\Nintedanib\SUNITINIB\IMATINIB\PAZO |
| | Sunitinib malate | PANIB\CRENOLANIB\DOVITINIB\MASITINIB\MOTESANIB\XL- |
| | (Sutent); | 820\AMUVATINIB\TANDUTINIB\X-82\TAK- |
| | Pazopanib HCl | 593\BECAPLERMIN\MIDOSTAURIN\AXITINIB\LINIFANIB\TELATINIB\VATALANIB\CE |
| | (Votrient); | DIRANIB\TSU-68\XL-999\Imatinib sublingual |
| | Regorafenib | |
| | (Stivarga); | |
| | Nintedanib esylate | |
| | (Ofev) | |
| PIK3CA | Copanlisib 2HCl | MIDOSTAURIN\ALPELISIB\PKI-587\PF- |
| | (Aliqopa); Alpelisib | 04691502\APITOLISIB\XL765\SF1126\COPANLISIB\944396-07-0\GDC-0941\PX- |
| | (Piqray) | 866\GSK2636771\XL147\PONATINIB\DASATINIB\BAY 80-6946\Buparlisib\GDC- |
| | | 0032\BYL719\LY3023414\MLN1117\BLY719\CH-5132799\INK-1117\PWT- |
| | | 33597\Pilaralisib\Alpelisib + Fulvestrant\Buparlisib + Fulvestrant\Fulvestrant + |
| | | Taselisib\Alpelisib\Buparlisib\Copanlisibl\GDC-0077\Serabelisib\Taselisib\PI3K-alpha |
| | | Inhibitor MEN1611\Sirolimus\Pictilisib\Akt Inhibitor MK2206\Idelalisib\Cabozantinib\ |
| | | Anti-EGFR Monoclonal Antibody\Gefitinib\Everolimus\Dactolisib\Radiation Therapy\ |
| | | Capivasertib\Lapatinib\Apitolisib\Ribociclib\Phosphatidylinositide 3-Kinase Inhibitor\ |
| | | Palbociclib\MTOR Kinase Inhibitor PP242\Fulvestrant\Everolimus\ |
| | | Perifosine\Temsirolimus\Afatinib\Erlotinib\Gefitinib\PI3K Inhibitor GDC-0941 |
| | | Bismesylate\PI103\Vemurafenib\Aspirin\Fulvestrant\AKT inhibitors\PI3K pathway |
| | | inhibitor (alone or in combination)s\PI3K pathway inhibitors\PIK3CA |
| | | inhibitors\IDELALISIB\TG100-115\AS-605240\MILTEFOSINE |
| PIK3CD | Idelalisib (Zydelig); | Duvelisib\ALPELISIB\PKI-587\PF- |
| | Copanlisib 2HCl | 04691502\APITOLISIB\XL765\SF1126\COPANLISIB\944396-07-0\GDC-0941\PX- |
| | (Aliqopa); Duvelisib | 866\GSK2636771\XL147\DELALISIB\MLN1117\TG100-115\AS-605240\BAY 80- |
| | (Copiktra); | 6946\PI-145\Buparlisib\INCB50465\TGR-1202\INCB40093\AMG 319\CH-5132799\GS- |
| | Umbralisib tosylate | 9820\GS-9901\ME-401\RP6530\XL-499\Wortmannin\Idelalisib (Zydelig) |
| | (Ukoniq) | |
| PIK3CG | Duvelisib (Copiktra) | Duvelisib\Staurosporine\ALPELISIBIPKI-587\PF- |
| | | 04691502\APITOLISIB\XL765\SF1126\COPANLISIB\944396-07-0\GDC-0941\PX- |
| | | 866\GSK2636771\XL147\PIK-93\DELALISIB\MLN1117\TG100-115\AS-605240\BAY |
| | | 80-6946\PI-145\Buparlisib\Enzastaurin\GDC-0032\Rigosertib\BEZ235\CLR- |
| | | 1404\GDC0941\GDC-0980/RG7422\PF-05212384\SAR245409\TGR- |
| | | 1202\BGT226\CLR-1401\CLR457\AZD8186\CH-5132799\CUDC-907\DS-7423\GDC- |
| | | 0077\GDC-0084\GSK2126458\PI-549\KA2237\P-7170\PA-799\Perifosine\VE-822\VS- |
| | | 5584\ZSTK474\GSK1059615\LY294002\SCR-44001\Wortmannin\AEZS- |
| | | 126\BAG956\CU-906\Dual PI3K/mTOR inhibitors\Her2-and PSA-targeted TGX-D1\HM- |
| | | 5016699\P-6915\PF-03772304\PF-4989216\PI-103\PKI-402\WX-037VIPI |
| | | 549\Fimepinostat\AZD 8186\Omipalisib\LY 294002 |
| PRKCA | Midostaurin | Midostaurin |
| | (Rydapt) | |
| PSMA7 | Bortezomib | CARFILZOMIB |
| | (Velcade) | |
| RAF1 | Sorafenib tosylate | MLN2480\LY3009120\RO-5126766\Sorafenib nanoparticle |
| | (Nexavar); | formulation\Dabrafenib\Regorafenib\GDC-5573\LErafAON\LXH254\RG7304\ISIS |
| | Regorafenib | 15770\PLX-ORI3\Cobimetinib\Regorafenib (Stivarga)\Sorafenib |
| | (Stivarga) | (Nexavar)\Paclitaxel\Carboplatin\Sorafenib\Pan-RAF inhibitors\Sorafenib (Pan-TK |
| | | inhibitor)\U0126 (MEK inhibitor)\BMS-908662\SORAFENIB TOSYLATE |
| RET | Vandetanib | Ponatinib\Sorafenib nanoparticle |
| | (Caprelsa); | formulation\Erdafitinib\Cabozantinib\Regorafenib\Lenvatinib\Amuvatinib\Imatinib\Imatinib |
| | Regorafenib | sublingual\SORAFENIB TOSYLATE\SUNITINIB\VANDETANIB\Romiplostim\CEP- |
| | (Stivarga); | 32496\MGCD516\BLU-667\LOXO-292\tamatinib\GW-559768XVITRI-305\MG- |
| | Cabozantinib S- | 516\Pralsetinib\Vandetanib\LOXO-292\BLU-667\Cabozantinib (Cabometyx [tablet]\ |
| | malate (Cometriq); | Cometriq [capsule])\Regorafenib (Stivarga)\Vandetanib (Caprelsa)\Motesanib\JAK2 |
| | Ponatinib HCl | Inhibitor AZD1480\Sorafenib\Sunitinib\Nintedanib\Everolimus\Cabozantinib\ |
| | (Iclusig); Lenvatinib | Alectinib\RET inhibitors\Cabozantinib (Pan-kinase inhibitor)\Nintedanib (Pan-TK |
| | mesylate (Lenvima); | inhibitor)\Sunitinib (Pan-TK inhibitor)\Vandetanib (Pan-TK inhibitor) |
| | Selpercatinib | |
| | (Retevmo); | |
| | Pralsetinib | |
| | (Gavreto) | |
| ROS1 | Ceritinib (Zykadia); | Entrectinib\CABOZANTINIB\Crizotinib\Lorlatinib\RXDX 101\TPX-0005\DS- |
| | Brigatinib | 6051\Imidazo[1\2-b]pyridazine derivative 1\2-b]pyridazine derivative 2\2-b]pyridazine |
| | (Alunbrig); | derivative 3\PMID28270010-Compound-FIG. 21-b\ASP3026\Crizotinib (Xalkori)\ |
| | Lorlatinib | Cabozantinib\Ceritinib\Foretinib\Brigatinib\Ceritinib\AZD3463\Lorlatinib\TAE684\ |
| | (Lorbrena); | Entrectinib\HSP90 inhibitors\Cabozantinib (Pan-kinase inhibitor)\Crizotinib (ALK |
| | Entrectinib | inhibitor)\Lorlatinib (ALK&ROS1 inhibitor) |
| | (Rozlytrek) | |
| SMO | Vismodegib | Glasdegib\VISMODEGIB\PF-04449913\LDE225\LY2940680\BMS-833923\LEQ- |
| | (Erivedge); | 506\TAK-441\Hedgehog pathway antagonists\SMOi2-17\Smoothened receptor |
| | Sonidegib | antagonist\BMS 833923\Cyclopamine\PF04449913\Sonidegib (Odomzo)\Vismodegib |
| | phosphate | (Erivedge)\PSI\Arsenic Trioxide\ Patidegib\Sonidegib\Vismodegib (SHH |
| | (Odomzo); | inhibitor)\Sonidegib\Patidegib\Taladegib |
| | Glasdegib maleate | |
| | (Daurismo) | |
| TOP2A | Teniposide | Elsamitrucin\Liposome encapsulated epirubicin\Epirubicin micelle |
| | (Vumon); | formulation\Epirubicin\Aldoxorubicin\Amrubicin\Etoposide |
| | Dexrazoxane HCl | phosphate\Etoposide\DOXORUBICIN\CAMPTOTHECIN\IDARUBICIN\PK1\PODOPHYL |
| | (Zinecard); | LOTOXIN\Anthracyclines (Chemotherapy)\Annamycin liposomal\Valrubicin\Doxorubicin |
| | Topotecan HCl | nanoparticles\Doxorubicin liposomal\Doxorubicin-eluting beads\Doxorubicin |
| | (Hycamtin); | intratumoural\Doxorubicin oral\Doxorubicin eosomes\Doxorubicin encapsulated |
| | Epirubicin HCl | nanoparticles\Doxorubicin inhaled\Doxorubicin drug delivery system\MTC |
| | (Ellence) | DOX\Amsacrine\Aclarubicin\Dexrazoxane\Dhaq |
| | | diacetate\Lucanthone\Mitoxantrone\Teniposide\Pixantrone\Tirapazamine\MTC-DOX\13- |
| | | DEOXYADRIAMYCIN HYDROCHLORIDE\Berubicin\CAP-7.1\GPX-150D\INNO- |
| | | 206\Liposomal encapsulated doxorubicin |
| | | (LED)\PYRAZOLOACRIDINE\Sabarubicin\Salvicine\SNS-595\XR-5000\F-14512\L- |
| | | 377202\Daniquidone\Iboctadekin + Doxil\MLN-576\NK-314\Tafluposide\TAS-103\XR- |
| | | 5944\BW-773U82\ELINAFIDE MESILATE\GL-331\KW- |
| | | 2170\LOSOXANTRONE\Merbarone\Mitonafide\NC-190\SUPER-LEU- |
| | | DOX\Banoxantrone\Teloxantrone\TOP-53\Nemorubicin\A-74932\BE-22179\Datelliptium |
| | | chloride\Doxorubicin-CEA conjugate\Liposome encapsulated mitoxantrone\Mitoxantrone |
| | | liposomal\Vosaroxin\BMS 247615\A 74932\Elinafide\PD 115934\KW 2170\GL 331\BE |
| | | 22179\AmonafidelBecatecarin\VORELOXINIRTA 744 |
| TUBA4A | Paclitaxel (Taxol); | Cabazitaxel\Cabazitaxel injection\Cabazitaxel nanosomal\VINCRISTINE |
| | Vinorelbine tartrate | |
| | (Navelbine); | |
| | Docetaxel | |
| | (Taxotere); | |
| | Ixabepilone | |
| | (Ixempra); | |
| | Cabazitaxel | |
| | (Jevtana kit); | |
| | Eribulin mesylate | |
| | (Halaven) | |
| TUBB | Paclitaxel (Taxol); | VINBLASTINE\VINCRISTINE\Vinorelbine liposomal\Vinorelbine\Vinorelbine |
| | Vinorelbine tartrate | emulsion\Vincristine liposomal\Vincristine sulfate liposomal |
| | (Navelbine); | |
| | Docetaxel | |
| | (Taxotere); | |
| | Ixabepilone | |
| | (lxempra); | |
| | Cabazitaxel | |
| | (Jevtana kit); | |
| | Eribulin mesylate | |
| | (Halaven) | |
| PSMB5 | Bortezomib | Marizomib\Carfilzomib\Bortezomib |
| | (Velcade) | |
| AKT1 | | Enzastaurin\GDC-0068\AZD5363\CI-1033\GSK2110183\PTX-200\RG7440\RX- |
| | | 0201\Trametinib + 2141795\Triciribine prodrug\ARQ 092\ARQ 751\M2698\AKT inhibitors |
| | | [PMCID:PMC3086120]\AKT protein kinase inhibitors\ALM-301\ARRY- |
| | | 886\Lactoquinomycin\LD-101\NU-1001-41\VLI-27\Capivasertib\BMS |
| | | 754807\NELFINAVIR\Vemurafenib\Uprosertib\Capivasertib\Dabrafenib\AKT inhibitors\ |
| | | allosteric AKT inhibitors\non-allosteric AKT inhibitors\PI3K pathway inhibitors\ |
| | | Everolimus (MTOR inhibitor)\Tensirolimus (MTOR inhibitor)\MNX-100\PerifosinelARQ- |
| | | 092\TRICIRIBINE\UNII-OLJ18SRI8A\AFURESERTIB HYDROCHLORIDE\TRICIRIBINE |
| | | PHOSPHATE |
| ATM | | AZD0156\AZD1390\M3541\VE-822\KU-60019\AZD 0156\M |
| | | 3541\Olaparib\Temozolomide\KU-0060648\NU7441\Olaparib\Paclitaxel\ATR inhibitors\ |
| | | DNA-PKc inhibitors\PARP inhibitors\AZD6738 (ATR inhibitor)\Cisplatin |
| | | (Chemotherapy)\Olaparib (PARP inhibitor)\Temozolomide (Chemotherapy) |
| ATR | | AZD6738\BAY1895344\M4344\VE-822\VX-970\VE-821\Olaparib (PARP inhibitor)\ |
| | | Temozolomide (Chemotherapy) |
| BARD1 | | Olaparib |
| BCL2L1 | | ABT-263\Obatoclax\APG-1252\E-003\WEHI-0103122\Gossypol |
| BIRC5 | | ISIS 23722\CV-9201\DPX-Survivac\EZN-3042\SurVaxM\GATAPARSEN |
| | | [USAN:INN]\Taxane Compound\Platinum Compound\AT-406\LCL161\APG-1387 |
| BRCA1 | | olaparib\niraparib\rucaparib\talazoparib\Niraparib\Olaparib\Talazoparib\Vinorelbine\ |
| | | Platinum Compound\Taxane Compound\Cediranib\Carboplatin\Cisplatin\Rucaparib\ |
| | | Pol I Inhibitor CX5461\Quarfloxin\Oxaliplatin\Gemcitabine\Veliparib\PARP inhibitor + |
| | | Chemotherapys\PARP inhibitors\WEE1 inhibitors\PARP inhibitors (Olaparibletc)\ |
| | | Olaparib (PARP inhibitor)\Platinum Agent (Chemotherapy)\Rucaparib (PARP inhibitor)\ |
| | | Veliparib + Cisplatin (PARP inhibitor + Chemotherapy) |
| BRCA2 | | olaparib\niraparib\rucaparib\talazoparib\Niraparib\Olaparib\Talazoparib\Mitomycin\ |
| | | Platinum Compound\Cediranib\Cisplatin\Carboplatin\Pol I Inhibitor CX5461\Quarfloxin\ |
| | | Rucaparib\Gemcitabine\Veliparib\Iniparib\PARP inhibitor + Chemotherapys\PD1 Ab |
| | | inhibitors\PARP inhibitors (Olaparibletc)\Olaparib (PARP inhibitor)\Platinum Agent |
| | | (Chemotherapy)\Rucaparib (PARP inhibitor)\Veliparib + Cisplatin (PARP inhibitor + |
| | | Chemotherapy) |
| BRD4 | | TOZASERTIB\CPI-0610\NCB57643\(+)-JQ1\ABBV-744\AZD5153\GSK525762\OTX- |
| | | 015\PLX2853\TEN010\JQ1 |
| CDK2 | | Bosutinib\AT 7519\Seliciclib\AT7519\BAY 10-00394\PHA848125\Ro 31-7453\R- |
| | | roscovitine\TG02\AG-024322\AZD-5438\CYC065\PHA-793887\RGB-286638\SNS- |
| | | 032\SCH 727965\R547\ZK 304709\INOC-005\PD-0183812\CDK2 inhibitors\PHA- |
| | | 690509\SCH-546909\VER-54505\NU 2058\AZD |
| | | 5438\Bohemine\Olomoucine\Staurosporine\Alvocidib\Nanoflavopiridol |
| CDK7 | | Seliciclib\R-roscovitine\SNS-032\SY-1365\R547\ZK |
| | | 304709\THZ1\Alvocidib\Nanoflavopiridol |
| CDK9 | | Seliciclib\BAY 10-00394\P276-00\AZD4573\CYC065\RGB-286638\SNS- |
| | | 032\Flavopirido/\SCH 727965\ZK 304709\CYC 065\Dinaciclib\AZD |
| | | 4573\BAY1143572\BAY 1251152\Alvocidib\Nanoflavopiridol |
| CDKN2A | | GEMCITABINE\Abemaciclib\Palbociclib\Ribociclib\Afatinib\Alvocidib\ |
| | | LetrozolelPalbociclib\Linsitinib\CDK4/6 inhibitors\IIorasertib (AURKA-VEGF |
| | | inhibitor)\952021-60-2 |
| CHEK1 | | 7-HYDROXYSTAUROSPORINE\GEMCITABINE\952021-60- |
| | | 2\LY2603618\LY2606368\SCH-900776\UCN-01\AZD7762\GDC-0425\LY2606368 |
| | | MsOH H20\RG7741\XL844\PF-477736\RG7602\ARRY-575\Chk1-AIS-024\S- |
| | | 070\Prexasertib\AZD 7762\MK 8776\UCN 01\CHIR-124\Cisplatin\Olaparib\D05UGJ |
| CHEK2 | | 7-HYDROXYSTAUROSPORINE\952021-60-2\AZD7762\D05UGJE7850\XL844\CCT- |
| | | 241533\PV-1019\Olaparib (PARP inhibitor) |
| DHFR | | PEMETREXED\Leucovorin Calcium\Methotrexate Sodium\PIRITREXIM\L-MDAM\MDAM |
| | | (y-methylene-10-deazaaminopterin)\TNP-351\1954U89\TNP |
| | | 351\Trimetrexate\PRALATREXATE\Methotrexate microparticle |
| | | encapsulated\Methotrexate oral solution\Methotrexate\Methotrexate liposomal\MPI 5004 |
| ERBB3 | | VANDETANIB\XL647\AFATINIB\AZD-9291 MESYLATE\DACOMITINIB\BMS- |
| | | 599626\BMS-690514\CUDC-101\PELITINIB\SAPITINIB\MUBRITINIB\S- |
| | | 222611\LAPATINIB\MEHD-7945A\MM-121\MM-141\AMG888/U3- |
| | | 1287\LJM716\Sym013\Anti-HER3/EGFR DAF\AV-203\CDX-3379\Drug |
| | | 2849330\GSK2849330\KTN3379\MM-151\Recombinant human Erbb3 fragment |
| | | therapeutic tumor vaccine\REGN1400\RG7116\MP-RM-1\Soluble ErbB3 |
| | | theragnostics\SYM-011\EZN-3920\9F7-F11\Selumetinib\HER2 Inhibitor CP- |
| | | 7241714\Gefitinib\Afatinib\PI3K pathway inhibitor + MEK inhibitors\Afatinib |
| | | (ERBB2&EGFR inhibitor 2nd gen)\Lapatinib (ERBB2 inhibitor) |
| GART | | PELITREXOL\LY309887\AG2034\AG 2034\PEMETREXED |
| GSK3B | | LY 2090314\LY2090314\tideglusib\AR-A014418\TDZD-8\DM- |
| | | 204\ALSTERPAULLONE\COMPOUND 89S\STAUROSPORINE\INDIRUBIN-3′- |
| | | MONOXIME |
| HIF1A | | PT2385\HIF-1alpha\IT-101\ENMD-1198\EZN-2968\PX-478\PT 238512- |
| | | Methoxyestradiol\Sunitinib (Pan-TK inhibitor) |
| HRAS | | ISIS 13920\vemurafenib\dabrafenib\trametinib\Tipifarnib\Epidermal Growth Factor |
| | | Receptor Tyrosine Kinase Inhibitor\Binimetinib\MTOR Kinase Inhibitor |
| | | AZD8055\Selumetinib\MEK Inhibitor PD0325901\Everolimus\Vemurafenib\MEK inhibitor |
| | | +/− MTOR inhibitors\MTOR inhibitors\PI3K pathway inhibitor + MEK inhibitors\Tipifarnib |
| | | (Farnesyltransferase inhibitor) |
| KIF11 | | ARRY-520\AZD-4877\spinesib\LY2523355\SB-743921\4SC-205\ARQ 621\MK- |
| | | 0731\ALN-VSP\EMD-534085\Filanesib |
| MAPK1 | | Seliciclib\Olomoucine\MARIMASTAT\PRINOMASTATI\BVD-523\GDC- |
| | | 0994\LY3214996\AEZS-131\ERK inhibitors\Erlotinib\WZ4002\Ulixertinib |
| MAPK3 | | Seliciclib\BVD-523\ASN007\GDC-0994\Ulixertinib |
| MCL1 | | Imatinib\MCL-1-specific antisense oligonucleotide + midostaurin(PKC412)\MCL-1- |
| | | specific antisense oligonucleotide + nilotinib (AMN107)\Obatoclax\AMG |
| | | 176\AZD5991\EU-517\AZD 5991 |
| NAMPT | | Daporinad\CHS-828\GMX1777\CHS 828 |
| NAPRT | | GNE-617 |
| NRAS | | GI-4000\Mutant ras vaccine\Reovirus\Salirasib\Perillyl alcohol\APC-300\KA-10XINEO |
| | | 102\vemurafenib\dabrafenib\trametinib\Binimetinib\Binimetinib + Ribociclib\Iodine I 131- |
| | | 6-Beta-lodomethyl-19-Norcholesterol + Selumetinib\Cobimetinib\Tanespimycin\ |
| NTRK1 | | Temozolomide\MEK Inhibitor RO4987655\Everolimus\Docetaxel\Binimetinib\ |
| | | Trametinib\Metformin\Omipalisib\Selumetinib\Amuvatinib\Dactolisib\Selumetinib\ |
| | | Ribociclib\Vemurafenib\CDK4/6 inhibitor + MEK inhibitors\ERK inhibitors\HSP90 |
| | | inhibitors\MEK inhibitor +/− PI3K pathway inhibitors\MEK inhibitors\Pan-RAF inhibitors\ |
| | | PI3K pathway inhibitor + MEK inhibitors\Sorafenib + MEK inhibitor (Pan-TK inhibitor + |
| | | MEK inhibitor) |
| | | Entrectinib\Larotrectinib\Regorafenib\AZD-6918\RXDX 101\ONO-7579\TPX- |
| | | 0005\Altiratinib\DS-6051\LOXO-101\LOXO-195\PLX7486\3-amino-5-benzyl-substituted |
| | | indazole derivative 1\Azaindazole amide derivative 1\Benzimidazole derivative 6\Bicyclic |
| | | heteroaryl benzamide derivative 1\Bicyclic heteroaryl benzamide derivative 2\Bicyclic |
| | | heteroaryl benzamide derivative 3\Bicyclic heteroaryl benzamide derivative 4\Bicyclic |
| | | heteroaryl benzamide derivative 5\Bicyclic heteroaryl benzamide derivative 6\Bicyclic |
| | | heteroaryl benzamide derivative 7\Bicyclic heteroaryl benzamide derivative 8\Bicyclic |
| | | heteroaryl benzamide derivative 9\Five membered heterocyclic benzamide derivative |
| | | 1\Five membered heterocyclic benzamide derivative 2\Five membered heterocyclic |
| | | benzamide derivative 3\Imidazo pyridine derivative 1\Imidazo[1\2-b]pyridazine derivative |
| | | 1\2-b]pyridazine derivative 2\2-b]pyridazine derivative 3\N-(phenylpyrazolyl)benzamide |
| | | derivative 1\N-acylpiperidine ether derivative 1\N-acylpiperidine ether derivative 2\N- |
| | | acylpiperidine ether derivative 3\N-acylpiperidine ether derivative 4\N-acylpiperidine |
| | | ether derivative 5\N-acylpiperidine ether derivative 6\N-acylpiperidine ether derivative |
| | | 7\N-acylpyrrolidine ether derivative 1\N-acylpyrrolidine ether derivative |
| | | 2\PMID28270021-Compound-WO2010077680 103\PMID28270021-Compound- |
| | | WO2010077680 109\PMID28270021-Compound-WO2010077680 201\PMID28270021- |
| | | Compound-WO2010077680 481\PMID28270021-Compound-WO2010077680 |
| | | 495\PMID28270021-Compound-WO2010077680 803\PMID28270021-Compound- |
| | | WO2010077680 811\PMID28270021-Compound-WO2013009582\PMID28270021- |
| | | Compound-WO2013009582Example 16\PMID28270021-Compound- |
| | | WO2013009582Example76\PMID28270021-Compound-WO2013161919Example85- |
| | | 117\PMID28270021-Compound-WO2013176970\PMID28270021-Compound- |
| | | WO2014078408Example1\PMID28270021-Compound- |
| | | WO2014078408Example26\PMID28270021-Compound-WO2014129431Example8- |
| | | 1\PMID28270021-Compound-WO2014152663 701\PMID28270021-Compound- |
| | | WO2015042088Example11\PMID28270021-Compound- |
| | | WO2015042088Example12\PMID28270021-Compound- |
| | | WO2015042088Example4\PMID28270021-Compound- |
| | | WO2015170218Example557\PMID28270021-Compound- |
| | | WO2016054807\PMID28270021-Compound- |
| | | WO2016054807Example1\PMID28270021-Compound- |
| | | WO2016054807Example112\PMID28270021-Compound- |
| | | WO2016054807Example71\PMID28270021-Compound- |
| | | WO2016054807Example80\PMID28270021-Compound- |
| | | WO2016054807Example82\Pyrazolo[1\5-a]pyridine derivative 115-a]pyridine derivative |
| | | 2\Pyrazolo[4\3-c]pyridine derivative 1\3-h]quinazoline-3-carboxamide derivative |
| | | 1\Pyrido[3\2-d]pyrimidine derivative 1\2-d]pyrimidine derivative 2\2-d]pyrimidine |
| | | derivative 3\Pyrrolo[2\3-b]pyridine derivative 1\3-b]pyridine derivative 2\3-b]pyridine |
| | | derivative 3\3-d]pyrimidine derivative 3\3-d]pyrimidine derivative 4\3-d]pyrimidine |
| | | derivative 5\Pyrrolo[3\2-c]pyridine derivative 1\Six-membered heterocyclic benzamide |
| | | derivative 1\Six-membered heterocyclic benzamide derivative 2\Six-membered |
| | | heterocyclic benzamide derivative 3\Six-membered heterocyclic benzamide derivative |
| | | 4\Six-membered heterocyclic benzamide derivative 5\Six-membered heterocyclic |
| | | benzamide derivative 6\Six-membered heterocyclic benzamide derivative 7\Triazolo[4\3- |
| | | b]pyridazine derivative 2\Tri-substituted urea derivative 1\Tri-substituted urea derivative |
| | | 2\AZD6918\Selitrectinib\larotrectinib sulfatel Entrectinib\ |
| | | Larotrectinib\Lestaurtinib\Crizotinib\Pan-TK inhibitors\Pan-TKR inhibitors\Pan-TK |
| | | inhibitors (Entrectinibletc)\Crizotinib (ALK inhibitor)\Imatinib\Imatinib sublingual |
| NTRK2 | | Entrectinib\Larotrectinib\AZD-6918\RXDX 101\LOXO-101\3-amino-5-benzyl-substituted |
| | | indazole derivative 1\Azaindazole amide derivative 1\PMID28270021-Compound- |
| | | WO2015042088Example4\PMID28270021-Compound- |
| | | WO2016054807Example1\Pyrrolo[2\3-d]pyrimidine 4\cabozantinib\larotrectinib sulfate\ |
| | | Entrectinib |
| NTRK3 | | Entrectinib\Larotrectinib\AZD-6918\RXDX 101\Altiratinib\LOXO-101\3-amino-5-benzyl- |
| | | substituted indazole derivative 1\Azaindazole amide derivative 1\PMID28270021- |
| | | Compound-WO2015042088Example4\PMID28270021-Compound- |
| | | WO2016054807Example1\Pyrrolo[2\3-d]pyrimidine derivative 4\CEP-2563\larotrectinib |
| | | sulfate\Entrectinib\Larotrectinib\Fms/Trk Tyrosine Kinase Inhibitor PLX7486 |
| | | Tosylatel\GF1R inhibitors\PI3K pathway inhibitors\Midostaurin (Pan-TK inhibitor)\novel |
| | | TRK inhibitors |
| PALB2 | | Mitomycin\Olaparib\PARP inhibitors\Mytomycin C (Chemotherapy)\Platinum Agent |
| | | (Chemotherapy)\OLAPARIB |
| PIM1 | | Staurosporine\SEL-24\CXR-1002\Pim1 inhibitors\Small molecule 18a\LY 294002 |
| PIM2 | | LGH-447\AZD1208\AZD 1208 |
| POLR2B | | Lurbinectedin |
| PRKCB | | Enzastaurin\RUBOXISTAURIN HYDROCHLORIDE\LY333531 |
| PRKCG | | Midostaurin\Research programme: MKNK1/MKNK2 protein inhibitors\Balanol |
| PTK2 | | VS-6063\PF-4554878\BI-853520\CEP-37440\GSK-2256098\PF-562271\VS-4718\PND- |
| | | 1186\Defactinib |
| PTPN11 | | TNO155 |
| RAD51 | | Amuvatinib |
| RAD51C | | PARP inhibitors |
| RPL3 | | Omacetaxine mepesuccinate |
| SF3B1 | | H3B-8800\Spliceostatin A\Spliceosome inhibitors |
| SRSF2 | | H3B-8800\Spliceosome inhibitors |
| TGFBR1 | | Galunisertib\LY2157299\LY3200882\P-2745\TEW-7197\GW-788388\TGF-beta receptor |
| | | 1 kinase inhibitor\TGF-beta receptor 1 kinase inhibitors\TGF-beta Shield\LY |
| | | 3200882\Research programme: transforming growth factor beta-1 antagonists |
| TSC1 | | Everolimus\Sirolimus\Everolimus\MTOR inhibitors\Everolimus (MTOR inhibitor) |
| TSC2 | | Everolimus\MTOR inhibitors\SRC inhibitors\Everolimus (MTOR inhibitor)\Tensirolimus |
| | | (MTOR inhibitor) |
| TYK2 | | AT9283 |
| TYMS | | Fluorouracil\TS antisense + raltitrexed\Plevitrexed (R)- |
| | | isomer\CAPECITABINE\FLOXURIDINE\RALTITREXED\TCMDC- |
| | | 131928\LEUCOVORIN\Irinotecan\Pemetrexed\ |
| | | Raltitrexed\Pemetrexed\GEMCITABINE\ENILURACIL |
| ERCC2 | | Cisplatin\Carboplatin\Paclitaxel\Cisplatin (Chemotherapy) |
| KDM6A | | Tazemetostat |
| SMARCB1 | | Tazemetostat\EZH2 inhibitors\HDAC inhibitors\VISMODEGIB |
| TUBG1 | | VINBLASTINE |
| U2AF1 | | H3B-8800\FLT3 inhibitors |

TABLE 10B
Exemplary therapeutic agents with associated IgSF-related targets or gene products

| | | Mechanism |
|---|---|---|
| IgSF | Drug-name (from CID) | of Action |
| CD2 | alpha-D-Mannose\|H-D-Cys(1)-D-Ala-Gln-D-Phe-Arg-D-Lys-Glu-D-Lys-Glu-D-aThr-Phe-Cys(1)- | Inhibitor\|. |
| | OH\|H-DL-Pen(1)-Ile-D-Tyr-Asp-D-aThr-Lys-Gly-Lys-D-Asn-Val-D-Leu-D-Cys(1)-OH\|H-Glu(1)-Ile- | |
| | Tyr-Asp-Pro-Gly-Asp-Asp-Ile-Lys(1)-OH\|cyclo[D-Asp-Asp-Ile-Lys-Glu-Ile-Tyr-Asp-Pro-Gly]\|H- | |
| | Glu(1)-Ser-Ile-Tyr-Asp-Pro-Gly-Asp-Asp-Ile-Lys(1)-OH\|Dimethyl fumarate | |
| IL1AP | Pulrodemstat | Inhibitor |
| FCG2B | N-hydroxy-4-[(2S)-2-phenylbutanoyl]amino]benzamide\|3-[(2-nitrophenyl)sulfanylmethyl]-4-prop- | Modulator\|. |
| | 2-enyl-1H-1,2,4-triazole-5-thione\|Epetraborole | |
| LIGO1 | N-[6-oxo-6-[(3S,4S)-3-[(4-phenoxyphenyl)sulfonylamino]-4-sulfanylpyrrolidin-1- | . |
| | yl]hexyl]acetamide | |
| FCGRN | H-Arg-Phe-Pen-Thr-Gly-His-Phe-Gly-Sar-N(Me)Leu-Tyr-Pro-Cys-OH\|(E)-3-(2-(1H-Tetrazol-5- | Inhibitor\| |
| | YL)vinyl)-6-fluoro-1H-indole | Antagonist |
| NTRK1 | K-252a analogue\|5-lodo-3-[(3,5-dibromo-4-hydroxyphenyl)methylene]-2-indolinone\|2- | Inhibitor\| |
| | Isopropoxy-12-(2-hydroxyethyl)-13,14-dihydronaphthol[2,1-a]pyrrolo[3,4-c]carbazole-5-one\|2- | Antagonist\| |
| | Methoxy-12-(2-hydroxyethyl)-13,14-dihydronaphthol[2,1-a]pyrrolo[3,4-c]carbazole-5- | Agonist |
| | one\|Sulfameter\|Fascaplysin\|6-Pyridin-3-yl-1,2,3,4-tetrahydronaphthalen-2-ol\|N-Cyclobutyl-N- | |
| | ((2,2-dimethyl-2H-pyrano[3,2-b]pyridin-6-yl)methyl)-3,4-dimethoxybenzenesulfonamide\|2-(3- | |
| | fluorophenyl)-1-(2-methyl-1-pentyl-1H-indol-3-yl)ethanone\|T-kinin\|N-[5-(4-tert-butylphenyl)-1,3,4- | |
| | thiadiazol-2-yl]-N-ethylpyridine-3-carboxamide\|(3S,4S,5S)-5-(Hydroxymethyl)piperidine-3,4- | |
| | diol\|2-Phenyl-5-(2-methylthiazol-4ylethynyl)pyridine\|5-[4-[2-(4-oxo-2H-1,3-benzoxazin-3- | |
| | yl)ethoxy]phenyl]methyl]-1,3-thiazolidine-2,4-dione\|Clothiapine\|Akt inhibitor VIII\|6-[6-(2-methyl- | |
| | 1H-pyrazol-2-ium-4-yl)-[1,2,4]triazolo[4,3-b]pyridazin-3-yl]sulfanyl]quinoline\|Unii- | |
| | VH3F3DR5X3\|3-((1H-imidazol-4-yl)methyl)pyridine\|1-(2-Methyl-1-pentyl-1H-indol-3-yl)-2-(4- | |
| | methylphenyl)ethanone\|2-[1,4]Diazepan-1-yl-6-nitro-quinoline\|3-[(3S)-3-ethyl-1-[8-[(3S)-3-ethyl- | |
| | 3-(3-hydroxyphenyl)azepan-1-yl]octyl]azepan-3-yl]phenol\|Isovelleral\|2-(3-Piperidin-1-yl-propyl)- | |
| | 3H-quinazolin-4-one\|3-(3-Adamantan-1-yl-ureido)-benzoic acid\|(5-Phenyl-thiazol-2-yl)-(4-(2- | |
| | pyrrolidin-1-yl-ethoxy)-phenyl)-amine\|8-O-(4-toluenesulfonyl)manzamine A\|(2S)-1-[(2R,3S)-3- | |
| | amino-2-hydroxy-5-methylhexanoyl]-N-[(2S)-1-amino-1-oxopropan-2-yl]pyrrolidine-2- | |
| | carboxamide\|Oritavancin\|Gusperimus trihydrochloride\|Tegaserod\|methyl 4-[4-[4-(1,1,3-trioxo- | |
| | 1,2-benzothiazol-2-yl)butyl]piperazin-1-yl]-1H-indole-2-carboxylate\|2-Phenyl-3-(piperidin-4-YL)- | |
| | 1H-indole\|(3R)-1-Acetyl-3-methylpiperidine\|Belizatinib\|(12Z)-9,10-Dihydroxyoctadec-12-enoic | |
| | acid\|(3S)-5-chlorospiro[2,4-dihydro-1H-naphthalene-3,4′-5H-1,3-oxazole]-2′-amine\|N-(5- | |
| | Benzyloxy-isoquinolin-1-yl)-guanidine\|2-[1-(2-Methylphenyl)pyrrol-3- | |
| | yIlethanamine\|[3H]robalzotan\|(2S)-N-[(2S,3S)-1-amino-3-methyl-1-oxopentan-2-yl]-1-[(2R)-1- | |
| | [(2S)-2-[(2R)-2-[(2S)-2-[(2S)-2-(benzoylamino)propanoyl]amino]propanoyl]amino]-3-(1H-indol- | |
| | 3-yl)propanoyl]amino]-3-phenylpropanoyl]pyrrolidine-2-carbonyl]pyrrolidine-2- | |
| | carboxamide\|cacospongionolide E\|Mirogabalin\|Hexylene | |
| | glycol\|Proguanil\|Aekkdegpyrmehfrwgsppkd\|[(2R)-1-[5-(3-tert-butylphenyl)-1H-indol-2- | |
| | yl]methyl]pyrrolidin-2-yl]methanol\|7-[3-(4-Acetyl-3-hydroxy-2-propylphenoxy)-2-hydroxypropoxy]- | |
| | 4-oxo-8-propyl-4H-1-benzopyran-2-carboxylic acid sodium salt\|SB 221284\|Para- | |
| | Bromoamphetamine\|N-(4-phenoxyphenyl)quinazolin-4-amine\|Idraparinux\|S-Sulfocysteine\|1- | |
| | Benzo[b]thiophen-2-yl-cycloheptylamine\|Levopropoxyphene napsylate anhydrous\|2-bromo-N-(2- | |
| | cyclopropyl-4-oxoquinazolin-3-yl)-5-methoxybenzamide\|Tirofiban\|Afegostat tartrate\|2- | |
| | Hydroxydodecanoic acid\|1,3-Dihydro-imidazo[4,5-b]quinolin-2-one\|1-Benzhydryl-4- | |
| | cyclopropylpiperidin-4-ol\|6-Methyl-4-(4-o-tolylpiperazin-1-yl)coumarin\|example 2 | |
| | [WO2013004676]\|N-{4″-[(trifluoromethyl)sulfonyl]-4-biphenyl}urea\|Tigapotide\|Amn082 | |
| | dihydrochloride\|Cyclohexylamine\|Promegestone\|5-(4-phenylphenyl)-2-undecan-6-yl-1H- | |
| | imidazole\|Glucagon-(1-6)\|9H-Pyrrolo[1′,2′:2,3]isoindolo[4,5,6-cd]indol-9-one,10-acetyl- | |
| | 2,6,6a,7,11a,11b-hexahydro-11-hydroxy-7,7-dimethyl-,(6aR,11aS,11bR)-\|3-[4-[(2S)-2- | |
| | aminopropoxy]phenyl]-N-[(1R)-1-(3-fluorophenyl)ethyl]imidazo[1,2-b]pyridazin-6-amine\|5-{3-[6- | |
| | Methyl-4-(4-trifluoromethyl-phenyl)-pyridin-2-yl]-phenyl}-pyridin-2-ylamine\|Toluene\|3-[4-[(2S)- | |
| | azetidin-2-yl]methoxy]phenyl]-N-[(1R)-1-(3-fluorophenyl)ethyl]imidazo[1,2-b]pyridazin-6- | |
| | amine\|Cetraxate\|8-cyclopentyl-2,6-diphenyl-7H-purine\|3-amino-6-methyl-N-pyridin-2-ylpyrazine- | |
| | 2-carboxamide\|N-Benzyl-2-(toluene-4-sulfonylamino)-benzamide\|2-[6-(4-Chlorophenoxy)hexyl]- | |
| | 1-cyano-3-pyridin-4-ylguanidine\|4-Hexyl-5-methylbenzene-1,3-diol\|(3R,4S,6R)-2- | |
| | (hydroxymethyl)-6-methoxyoxane-3,4,5-triol\|Altiratinib\|Selitrectinib\|Tovinontrine\|Repotrectinib\|4- | |
| | hydroxy-6-nitro-3-phenylquinolin-2(1H)- | |
| | one\|Sakuranetin\|Pegcantratinib\|Tavilermide\|Entrectinib\|Larotrectinib\|U.S. Pat. No. 10,323,022, Example | |
| | 152\|Pcmbs\|(6R,13S)-9-fluoro-13-hydroxy-2,11,15,19,20,23- | |
| | hexazapentacyclo[15.5.2.17,11.02,6.020,24]pentacosa-1(23),7,9,17(24),18,21-hexaene-16,25-dione\| | |
| | U.S. Pat. No. 10,323,022, Example 610\|U.S. Pat. No. 10,323,022, Example 613JU.S. Pat. No. 10,323,022, | |
| | Example 612\|1-(trans-4-Phenyl-1-(2-(trifluoromethoxy)ethyl)pyrrolidin-3-yl)-3-(2-phenyl-2,4,5,6- | |
| | tetrahydrocyclopenta[c]pyrazol-3-yl)urea\|Methyl 3-methoxy-2-((trans)-3-phenyl-4-(3-(2-phenyl- | |
| | 2,4,5,6-tetrahydrocyclopenta[c]pyrazol-3-yl)ureido)pyrrolidin-1-yl)propanoate\|U.S. Pat. No. 10,323,022, | |
| | Example 130\|U.S. Pat. No. 10,323,022, Example 317\|U.S. Pat. No. 10,323,022, Example 312\|U.S. Pat. No. | |
| | 10,323,022, Example 495\|U.S. Pat. No. 10,323,022, Example 194\|Delorazepam\|U.S. Pat. No. 10,323,022, | |
| | Example 61\|2-Methoxy-4-(m-tolyloxy)benzonitrile\|N-cyclopropyl-5-((2R,4R)-2-(3-fluorophenyl)- | |
| | 4-hydroxypyrrolidin-1-yl)pyrazolo[1,5-a]pyrimidine-3-carboxamide\|(R)-Larotrectinib\|(R)- | |
| | N-cyclopropyl-5-(2-(5-fluoro-2-(2-morpholinoethoxy)phenyl)pyrrolidin-1-yl)pyrazolo[1,5-a] | |
| | pyrimidine-3-carboxamide\|N-cyclobutyl-2-[3-[4-(1H-indazol-5-ylamino)-5,6-dimethylpyrimidin-2-yl] | |
| | phenoxy]acetamide\|(3S)-N-[5-[(2R)-2-(5-fluoro-2-methoxyphenyl)pyrrolidin-1-yl]pyrazolo[1,5-a] | |
| | pyrimidin-3-yl]-3-hydroxypiperidine-1-carboxamide\|(R,E)-3-(5-(2-(2,5-difluorophenyl)pyrrolidin-1-yl) | |
| | pyrazolo[1,5-a]pyrimidin-3-yl)-1-morpholinoprop-2-en-1-one\|2-Tert-butyl-5-(5-(2-(2,5-difluorophenyl) | |
| | pyrrolidin-1-yl)pyrazolo[1,5-a]pyrimidin-3-yl)-1,3,4-thiadiazole\|5-(3-Chlorophenylethynyl)-5- | |
| | methyl[1,2,4]triazine\|2-(4,4-difluoropiperidin-1-yl)-6-methoxy-N-(1-propan-2-ylpiperidin-4-yl)-7-(3- | |
| | pyrrolidin-1-ylpropoxy)quinazolin-4-amine\|1-cyano-N-[5-[(2R)-2-(2,5-difluorophenyl)pyrrolidin-1- | |
| | yl]pyrazolo[1,5-a]pyrimidin-3-yl]cyclopropane-1-carboxamide\|Revizinone\|(S)-N-(6-((R)-2-(2,5- | |
| | difluorophenyl)pyrrolidin-1-yl)imidazo[1,2-b]pyridazin-3-yl)-3-hydroxypyrrolidine-1- | |
| | carboxamide\|4-[3-amino-4-(1,1,1-trifluoropropan-2-yloxy)-1H-indazol-6-yl]-2-(4-phenylpiperazin- | |
| | 1-yl)benzamide\|4-[3-amino-4-(oxan-4-yloxy)-1H-indazol-6-yl]-2-(4-phenylpiperazin-1- | |
| | yl)benzamide\|4-[3-amino-4-(oxan-4-yloxy)-1H-indazol-6-yl]-2-(4-cyclohexylpiperazin-1-yl)-N- | |
| | methylbenzamide\|(R)-N-(6-(2-(2,5-difluorophenyl)pyrrolidin-1-yl)imidazo[1,2-b]pyridazin-3-yl)-4- | |
| | (methylsulfonamido)benzamide\|N-cyclopropyl-5-((2R)-2-(3-(2,3-dihydroxypropoxy)-5- | |
| | fluorophenyl)pyrrolidin-1-yl)pyrazolo[1,5-a]pyrimidine-3-carboxamide\|U.S. Pat. No. 10,323,022, Example | |
| | 441\|U.S. Pat. No. 10,323,022, Example 511\|hTrkA-IN-1\|XX8E5WP7FS\|1-[3-(Dimethylamino)propyl]-3-[4- | |
| | [[5,5-dimethyl-2,4-dioxo-3-[4-(trifluoromethylsulfanyl)phenyl]imidazolidin-1-yl]methyl]pyridin-2- | |
| | yl]urea\|1-[4,5-Dichloro-2-(3-methoxypropyl)phenyl]-3-[4-methyl-5-(1-methylpyrazol-4-yl)-2- | |
| | phenylpyrazol-3-yl]urea\|N-[4-(4-aminophenyl)sulfanyl-6-(4-methylpiperazin-1-yl)-1,3,5-triazin-2- | |
| | yl]-5-methyl-1,3-thiazol-2-amine\|CID 90168931\|5,5-Dimethyl-1-[2-(pyridin-2-ylamino)pyridin-4- | |
| | yl]methyl]-3-[4-(trifluoromethylsulfanyl)phenyl]imidazolidine-2,4-dione\|CID 73891136\|1-[4-[4-(4- | |
| | Methylpiperazin-1-yl)-6-[(5-methyl-1,3-thiazol-2-yl)amino]-1,3,5-triazin-2-yl]sulfanyl]phenyl]-3- | |
| | phenylurea\|[6-Cyclopropylmethoxy-5-(3,3-difluoro-azetidin-1-yl)-pyridin-2-yl]-(2,2-dimethyl- | |
| | pyrrolidin-1-yl)-methanone\|5-(3-Methoxy-4-((4-methoxybenzyl)oxy)benzyl)pyrimidine-2,4- | |
| | diamine\|1-(1′,4-dimethyl-1-phenyl-1H,1′H-[3,4′-bipyrazol]-5-yl)-3-(5-(methoxymethyl)-2- | |
| | (trifluoromethyl)benzyl)urea | |
| VGFR3 | Sitravatinib\|N-(3-methylphenyl)-2-(4-pyridin-2-ylpiperidin-1-yl)acetamide\|(R)-2-amino-1-ethyl-7- | Inhibitor\| |
| | (3-hydroxy-4-methoxy-3-methylbut-1-yn-1-yl)-N-methyl-4-oxo-1,4-dihydro-1,8-naphthyridine-3- | Modulator |
| | carboxamide\|3-[(4-chlorophenyl)-(1H-imidazol-2-yl)methylidene]-5-[(1-ethylpiperidin-4-yl)amino]- | |
| | 1H-indol-2-one\|Pyrazolo[1,5-a]pyrimidine 1a\|N-[2,4-dichlorobenzoyl]phenylsulfonamide\|4- | |
| | Chloro-N-(2-chlorobenzoyl)benzenesulfonamide\|3-chloro-N-(4- | |
| | chlorophenyl)sulfonylbenzamide\|4-Chloro-N-[(4-chlorophenyl)sulfonyl]benzamide\|N,5-diphenyl- | |
| | 1,3-oxazol-2-amine\|2-[(5-Phenyl-1,3-oxazol-2-yl)amino]benzonitrile\|N-(2-methoxyphenyl)-5- | |
| | phenyl-1,3-oxazol-2-amine\|3-[(5-Phenyl-1,3-oxazol-2-yl)amino]benzonitrile\|3-(5-Phenyl-oxazol-2- | |
| | ylamino)phenylmethanol\|N-(4-chlorobenzenesulfonyl)-2-methylbenzamide\|3-bromo-N-(4- | |
| | chlorophenyl)sulfonylbenzamide\|N-(4-chlorophenyl)sulfonyl-4-nitrobenzamide\|N-(3- | |
| | methylphenyl)-5-phenyl-1,3-oxazol-2-amine\|4-[(5-Phenyl-1,3-oxazol-2- | |
| | yl)amino]benzenesulfonamide\|N-(4-chloro-2-fluorophenyl)-6,7-dimethoxyquinazolin-4-amine\|2- | |
| | Isopropoxy-12-(2-hydroxyethyl)-13,14-dihydronaphthol[2,1-a]pyrrolo[3,4-c]carbazole-5-one\|4- | |
| | Oxobutanoate\|CID 73755245\|MK- | |
| | 2461\|Vatalanib\|Famitinib\|Sulfatinib\|pentadecasodium; [(2R,3R,4S,5S,6R)-6-[(2R,3S,4S,5R,6R)- | |
| | 2-[(2R,3S,4S,5R,6R)-2-[(2R,3R,4S,5S,6R)-3,5-disulfonatooxy-2-(sulfonatooxymethyl)-6- | |
| | [(2R,3S,4R,5R,6R)-2,4,5-trisulfonatooxy-6-(sulfonatooxymethyl)oxan-3-yl]oxyoxan-4-yl]oxy-3,5- | |
| | disulfonatooxy-6-(sulfonatooxymethyl)oxan-4-yl]oxy-3,5-disulfonatooxy-6- | |
| | (sulfonatooxymethyl)oxan-4-yl]oxy-3,4,5-trisulfonatooxyoxan-2-yl]methyl phosphate\|N-(3,5- | |
| | dichlorophenyl)-1H-indazole-5-carboxamide\|Linifanib\|Lucitanib\|Fruquintinib\|Tivozanib\|Emetine | |
| RAGE | c[L-Phe-D-pro-L-Tyr(OMe)-D-trp]\|N-(3,3-diphenylpropyl)benzamide\|Atopaxar\|Alagebrium | Modulator\| |
| | chloride\|Azeliragon\|Pyridoxamine | Inhibitor\| |
| | | Breaker\| |
| | | Antagonist |
| LIRB2 | (1S,3aR,6aS)-2-((R)-2-((S)-2-cyclohexyl-2-(pyrazine-2-carboxamido)acetamido)-3,3- | Inhibitor |
| | dimethylbutanoyl)-N-((S)-1-(cyclopropylamino)-1,2-dioxohexan-3- | |
| | yl)octahydrocyclopenta[c]pyrrole-1-carboxamide | |
| CD79B | Acetic acid 2-pentylsulfanyl-phenyl ester\|N-hydroxy-4-[(2S)-2- | \|.Modulator\| |
| | phenylbutanoyllamino]benzamide\|Phenyl 4- | Agonist |
| | (undecyloxy)phenylcarbamate\|Cerivastatin\|N,N,N′,N′-Tetramethyl-p-phenylenediamine | |
| | dihydrochloride\|1-Aminomethyl-3-phenyl-isochroman-5,6-diol | |
| PTPRS | Vanadic acid\|Etidronic acid | Inhibitor |
| CD4 | 3-(4-Methoxyphenyl)-5H-indeno[1,2-c]pyridazin-5-one\|5-methoxy-N-[(4-methoxyphenyl)methyl]- | .\|Inhibitor\| |
| | 1H-indole-2-sulfonamide\|2-Methoxy-4-(p-tolyloxy)benzonitrile\|1-(((Z)-(1-(2-aminothiazol-4-yl)-2- | Binder\| |
| | oxo-2-(((3S,4R)-2-oxo-4-((2-oxooxazolidin-3-yl)methyl)-1-sulfoazetidin-3- | Modulator |
| | yl)amino)ethylidene)amino)oxy)cyclopropanecarboxylic acid\|1,2-(BiS(1,3-dithia-2- | |
| | bismolane)thio)ethane\|Vaborbactam\|CID 23712772\|5-Propyl-2′-deoxyuridine \|8-((2- | |
| | (Dimethylamino)ethyl)amino)-5-(hydroxymethyl)-9-oxo-9H-xanthen-2-yl 4- | |
| | methylbenzenesulfonate\|Cyclohexylmethylcarbamic Acid Biphenyl-3-yl Ester\|c[L-Phe-D-pro-L- | |
| | mTyr-D-trp]\|Cyclopropanecarboxamide, N,N′-((1,1′-biphenyl)-4,4′-diylbis(5,2-thiazolediyl-(2S)- | |
| | 2,1-pyrrolidinediyl((1R)-2-oxo-1-phenyl-2,1-ethanediyl)))bis-\|2-[5-(1,2-Oxazole-5-carbonyl)- | |
| | [1,2,4]triazino[5,6-b]indol-3-yl]sulfanylmethyl]benzonitrile | |
| ILRL2 | 6-Amino-2-(2-methoxyphenylthio)benzenecarbonitrile | Antagonist |
| VGFR2 | (2E)-2-{[5-(4-bromophenyl)furan-2-yl]methylidene}hydrazinecarbothioamide\|N-(3-(5-chloro-1H- | .\|Inhibitor\| |
| | pyrrolo[2,3-b]pyridine-3-carbonyl)-2,4-difluorophenyl)propane-1- | Antagonist\| |
| | sulfonamide\|Cyclopropanecarboxamide,N,N′-((1,1′-biphenyl)-4,4′-diylbis(5,2-thiazolediyl-(2S)- | Modulator\| |
| | 2,1-pyrrolidinediyl((1R)-2-oxo-1-phenyl-2,1-ethanediyl)))bis-\|Sitravatinib\|(E)-(thiophen-2- | Binder |
| | ylmethylidene)amino benzoate\|lodipamide\|H-DL-Tyr(2,6-diMe)-DL-Tic-DL-Lys(Cbz)-NHPh\|(E)-1- | |
| | (4-((6-amino-5-((methoxyimino)methyl)pyrimidin-4-yl)oxy)-2-chlorophenyl)-3-ethylurea\|alpha- | |
| | (((2-Bromoethyl)amino)methyl)-2-nitro-1H-imidazole-1-ethanol monohydrobromide\|1-(4-((6,7- | |
| | Dimethoxyquinolin-4-yl)oxy)-2-methoxyphenyl)-3-(1-(thiazol-2-yl)ethyl)urea\|VEGF Receptor 2 | |
| | Kinase Inhibitor I\|VEGF Receptor 2 Kinase Inhibitor Il\|Pyrazolo[1,5-a]pyrimidine,6-(4- | |
| | methoxyphenyl)-3-(3-thienyl)-\|N-(4-Chlorophenyl)-2-[(pyridin-4-ylmethyl)amino]benzamide\|1- | |
| | cyclopentyl-3-(1H-pyrrolo[2,3-b]pyridin-5-yl)-1H-pyrazolo[3,4-d]pyrimidin-4-amine\|4-(4-(3-(1H- | |
| | Pyrazol-4-yl)pyrazolo[1,5-a]pyrimidin-6-yl)phenyl)morpholine\|3-[(4-chlorophenyl)-(1H-imidazol-2- | |
| | yl)methylidene]-5-[(1-ethylpiperidin-4-yl)amino]-1H-indol-2-one\|1-(4-((4-Ethylpiperazin-1- | |
| | yl)methyl)-3-(trifluoromethyl)phenyl)-3-(4-((6-(methylamino)pyrimidin-4- | |
| | yl)oxy)phenyl)urea\|Nintedanib Impurity A;BIBF 1202\|N-[3-[5-lodo-4-[3-[(2- | |
| | thienylcarbonyl)amino]propyl]amino]-2-pyrimidinyl]amino]phenyl]-1-pyrrolidinecarboxamide\|N-[3- | |
| | [5-Bromo-4-[2-(1H-imidazol-5-YL)ethyl]amino]-2-pyrimidinyl]amino]phenyl]-1- | |
| | pyrrolidinecarboxamide\|Pyrazolo[1,5-a]pyrimidine 1a\|Benzimidazole 2\|N-(4- | |
| | phenoxyphenyl)quinazolin-4-amine\|2-Quinolinone deriv.3\|Benzoannulenone 1\|#N/A\|N-[2,4- | |
| | dichlorobenzoyl]phenylsulfonamide\|4-Chloro-N-(2-chlorobenzoyl)benzenesulfonamide\|3-chloro- | |
| | N-(4-chlorophenyl)sulfonylbenzamide\|4-Chloro-N-[(4-chlorophenyl)sulfonyl]benzamide\|N,5- | |
| | diphenyl-1,3-oxazol-2-amine\|2-[(5-Phenyl-1,3-oxazol-2-yl)amino]benzonitrile\|N-(2- | |
| | methoxyphenyl)-5-phenyl-1,3-oxazol-2-amine\|3-[(5-Phenyl-1,3-oxazol-2-yl)amino]benzonitrile\|N- | |
| | (3-phenoxyphenyl)-5-phenyl-1,3-oxazol-2-amine\|3-(5-Phenyl-oxazol-2- | |
| | ylamino)phenylmethanol\|N-(4-chlorobenzenesulfonyl)-2-methylbenzamide\|3-bromo-N-(4- | |
| | chlorophenyl)sulfonylbenzamide\|N-(4-chlorophenyl)sulfonyl-4-nitrobenzamide\|N-(3- | |
| | methylphenyl)-5-phenyl-1,3-oxazol-2-amine\|4-[(5-Phenyl-1,3-oxazol-2- | |
| | yl)amino]benzenesulfonamide\|3-methyl-1H-thieno[2,3-c]pyrazole-5-carboxamide\|N-(4-chloro-2- | |
| | fluorophenyl)-6,7-dimethoxyquinazolin-4-amine\|4-(4-(4- | |
| | (Chlorodifluoromethoxy)phenylamino)phthalazin-1-yl)benzamide\|4-(4-(4-Tert- | |
| | butylphenylamino)phthalazin-1-yl)benzamide\|4-(4-m-Tolylamino-phthalazin-1-yl)-benzamide\|4- | |
| | (4-p-Tolylamino-phthalazin-1-yl)-benzamide\|N,N-dimethyl-5-[14-oxo-7-(propan-2-yloxymethyl)- | |
| | 3,13-diazahexacyclo[14.7.0.02,10.04,9.011,15.017,22]tricosa- | |
| | 1(16),2(10),4(9),5,7,11(15),7,19,21-nonaen-3-yl]pentanamide\|4-[(2S)-2-(3-Chlorophenyl)-2- | |
| | hydroxyethyl]amino]-3-[7-methyl-5-(4-morpholinyl)-1H-benzimidazol-2-yl]-2(1H)-pyridinone\|K- | |
| | 252a analogue\|5-[(Z)-(5-chloro-2-oxo-1,2-dihydro-3H-indol-3-ylidene)methyl]-N-[2- | |
| | (diethylamino)ethyl]-2,4-dimethyl-1H-pyrrole-3-carboxamide\|Pyrazolo[1,5-a]pyrimidine 4e\|2- | |
| | (Pyridin-4-Ylmethylamino)-~{n}-[3-(Trifluoromethyl)phenyl]benzamide\|3-[5-(3-Thienyl)-3- | |
| | pyridinyl]-1H-indole\|Unii-3mae0mxi6K\|3-(4-Aminophenyl)thieno[3,2-c]pyridin-4-amine\|4-Phenyl- | |
| | 2-(phenylamino)pyrimidine-5-carbonitrile\|2-(p-Toluidino)-4-phenylpyrimidine-5-carbonitrile\|3-((3- | |
| | Bromothiophen-2-yl)methylene)indolin-2-one\|8-methyl-4H,7H-indolo[6,5,4-cd]indol-5-one\|4-[3- | |
| | Hydroxyanilino]-6,7-dimethoxyquinazoline\|3-Phenyl-1,4-dihydroindeno[1,2-c]pyrazole\|2-(4- | |
| | Methoxyphenyl)-3-phenylmaleimide\|2-(Pyrimidin-4-ylamino)thiazole-5-carbonitrile\|6-o- | |
| | Tolylquinazolin-2-amine\|4-(4-aminophenyl)-1H-indazol-3-amine\|(4-Chlorophenyl)-(4-isoquinolin- | |
| | 5-yl-phthalazin-1-yl)amine\|4-(isoquinolin-5-yl)-N-m-tolylphthalazin-1-amine\|4-(isoquinolin-5-yl)-N- | |
| | o-tolylphthalazin-1-amine\|3-(1H-Benzimidazol-2-YL)-1H-indazole\|7-(4-fluoroanilino)-1-[(1R,3R)- | |
| | 3-hydroxycyclopentyl]-3-(4-methoxyphenyl)-4H-pyrimido[4,5-d]pyrimidin-2-one\|1H-Pyrrole-2,5- | |
| | dione,3,4-bis(4-methoxyphenyl)-\|3,4-diphenyl-1H-pyrrole-2,5-dione\|5-isopropyl-6-(5-methyl- | |
| | 1,3,4-oxadiazol-2-yl)-N-(2-methyl-1H-pyrrolo[2,3-b]pyridin-5-yl)pyrrolo[2,1-f][1,2,4]triazin-4- | |
| | amine\|2-Methoxy-12-(2-hydroxyethyl)-13,14-dihydronaphthol[2,1-a]pyrrolo[3,4-c]carbazole-5- | |
| | one\|3-(5,6,7,13-Tetrahydro-9-((1-methylethoxy)methyl)-5-oxo-12h-indeno(2,1-a)pyrrolo(3,4- | |
| | c)carbazol-12-yl)propanol\|Erythribyssin L\|5-{[(3Z)-5-fluoro-2-oxo-2,3-dihydro-1H-indol-3- | |
| | ylidene]methyl}-N-[(2S)-2-hydroxy-3-(morpholin-4-yl)propyl]-2,4-dimethyl-3H-pyrrole-3- | |
| | carboxamide\|Motesanib\|Duloxetine\|Tyrosine kinase-IN-1\|VEGF receptor tyrosine kinase inhibitor | |
| | III\|N,2-Dimethyl-6-{[7-(2-Morpholin-4-Ylethoxy)quinolin-4-Yl]oxy}-1-Benzofuran-3- | |
| | Carboxamide\|(2R)-2-amino-3-[1-[3-[2-[4-[1,3-bis(2-methoxyethylcarbamoyloxy)propan-2- | |
| | yloxy]butanoylamino]ethylamino]-3-oxopropyl]-2,5-dioxopyrrolidin-3-yl]sulfanylpropanoic | |
| | acid\|Golvatinib\|Osi-930\|Tak-593\|4-Methyl-5-(2-(4-morpholinophenylamino)pyrimidin-4-yl)thiazol- | |
| | 2-amine\|Altiratinib\|Cep-11981\|8-fluoro-12-(4-methylpiperazin-1-yl)-6H-[1]benzothieno[2,3- | |
| | b][1,5]benzodiazepine\|(2R)-4-(1H-indol-3-yl)-2-[2-(4-piperidin-4- | |
| | ylbutanoylamino)acetyl]amino]butanoic acid\|Zanoterone\|MK-2461\|Fluconazole\|Delphinidin\|3-((4- | |
| | Bromo-2,6-difluorobenzyl)oxy)-5-(3-(4-(pyrrolidin-1-yl)butyl)ureido)isothiazole-4-carboxamide\|1- | |
| | Benzyl-3-hydroxy-4-[(1-phenylethylamino)methyl]pyridin-2-one\|(3R,4R)-4-Amino-1-[4-[(3- | |
| | methoxyphenyl)amino]pyrrolo[2,1-f][1,2,4]triazin-5-yl]methyl]piperidin-3-ol\|Vatalanib\|1-methyl-5- | |
| | [2-[5-(trifluoromethyl)-1H-imidazol-2-yl]pyridin-4-yl]oxy-N-[4-(trifluoromethyl)phenyl]benzimidazol- | |
| | 2-amine\|Foretinib\|Telatinib\|Famitinib\|Sulfatinib\|Cediranib\|Brivanib\|Lucitanib\|Fruquintinib\|Orantinib\| | |
| | Neratinib\|Tivozanib\|Lenvatinib\|Apatinib Mesylate\|Semaxanib\|Lanatoside | |
| | A\|Axitinib\|Sunitinib\|Sorafenib\|Regorafenib\|Pazopanib | |
| | hydrochloride\|Vandetanib\|Cabozantinib\|6,7-Dimethyl-2-phenylquinoxaline\|1H-3-Benzazepine,6- | |
| | chloro-2,3,4,5-tetrahydro-3-methyl-9-((3-methyl-2-butenyl)oxy)-\|N-benzyl-4-(5-formyl-6- | |
| | hydroxynaphthalen-2-yl)-N-methylbenzamide\|Tupichinol C\|2-(Tert-butylamino)-1-(3,4- | |
| | dichlorophenyl)pentan-1-one\|Bardoxolone methyl\|Almorexant\|BzATP\|Cyclopentylcytosine | |
| | triphosphate | |
| CTLA4 | Nastorazepide\|6-(4-Tert-butylphenoxy)pyridin-3-amine\|4-((4-(Hexylcarbamoyl)-2-(naphthalen-1- | .\|Inhibitor\| |
| | yl)oxazol-5-yl)methyl)phenyl dihydrogen phosphate\|2-Aminomethyl-5-(p-t- | Modulator\| |
| | butylphenyl)tetrahydrofuran\|N-(4-tert-butylphenyl)-4-chloro-3-[(3-chloropyridin-2-yl)-(2- | Agonist\| |
| | methoxyethyl)amino]benzamide\|alpha-D-Mannose\|L-Fucose\|8-(3-Methyl-butoxy)-quinolin-2- | Antagonist |
| | ylamine\|Dodecane-1-sulfonyl fluoride\|(5Z)-3-allyl-4-hydroxy-5-[(5-phenyl-2- | |
| | furyl)methylene]thiazolidine-2-thione\|#N/A\|Benzamide,4-(4-(((6-methoxy-2-(2- | |
| | methoxyimidazo(2,1-b)-1,3,4-thiadiazol-6-yl)-4-benzofuranyl)oxy)methyl)-2-thiazolyl)-N,N- | |
| | dimethyl-\|N-(3-phenoxy-4-pyridinyl)ethanesulfonamide\|Gallium | |
| | citrate\|\|bezapolstat\|Dichloroacetate\|(2S,3S)-2-Amino-3-methyl-N-(2- | |
| | morpholinoethyl)pentanamide\|2-(4-Morpholinyl)-8-phenyl-4H-1-benzopyran-4-one\|Sibofimloc\|4″- | |
| | (p-Fluorobenzyl)tylosin A\|LY 2880070 [Who-DD]\|3-[2-(2-amino-6-oxo-5H-purin-9-yl)ethyl-(2- | |
| | phosphonoethyl) amino]propanoic acid\|Unii-3NC9ase900 | |
| ICOSL | N-Benzyl,N-methyl-1H-indole-2-carboxamide\|Cadazolid | Modulator\| |
| | | Inhibitor |
| CEAM5 | Phenol\|6-hydroxymanzamine A\|Secobarbital\|2-{4-[(N-phenylpentanamido)methyl]phenyl}benzoic | CAR-T-Cell- |
| | acid\|Meriolin 2\|Oxytocin\|4-(3-Hydroxypiperidin-3-yl)benzene-1,2-diol\|3,5-Diiodo-L- | Therapy\|.\| |
| | tyrosine\|Prexasertib\|2-Propyl-pentanoic acid 4-sulfamoyl-benzylamide\|9,10,11,12-Tetrahydro- | Modulator\| |
| | 9,12-epoxy-1H-diindolo[1,2,3-fg:3′,2′,1′-kl]pyrrolo[3,4-i][1,6]benzodiazocine-1,3(2H)-dione\|N-[4- | Inhibitor\| |
| | [(3S)-3-aminopiperidin-1-yl]pyridin-3-yl]-2-(2,6-difluorophenyl)-1,3-thiazole-4-carboxamide\|(2S)- | Immunomodulator |
| | 2-[(2S)-2-[[(2S)-2-[[(2S,3R)-2-[[(2S)-2-[[(2S)-2-[(2S)-2-[[(2S)-2-[(2S)-2-[(2- | |
| | acetamidoacetyl)amino]propanoyl]amino]-5-amino-5-oxopentanoyl]amino]-3- | |
| | phenylpropanoyl]amino]-3-hydroxypropanoyl]amino]-6-aminohexanoyl]amino]-3- | |
| | hydroxybutanoyl]amino]propanoyl]amino]propanoyl]amino]-6-aminohexanoic | |
| | acid\|Dichloroacetate\|Bopindolol\|2-(2-(2,5-Dichlorophenylamino)phenyl)acetic acid\|Lactulose\|N- | |
| | [1-[(2R,4R,5R)-5-(hydroxymethyl)-4-prop-2-ynoxyoxolan-2-yl]-2-oxopyrimidin-4- | |
| | yl]benzamide\|Peptide: CEA: 571-579 (576D)\|(3R)-5-(2,6-dichlorobenzoyl)oxy-3-[(2S)-2-[[(2S)-3- | |
| | methyl-2-(phenylmethoxycarbonylamino)butanoyl]amino]propanoyl]amino]-4-oxopentanoic | |
| | acid\|Ac-HSDAVFTDQYTRLRKQVAAKKYLQSIKQKRYC\|Protionamide\|7-Chloro-N-methyl-5-(1H- | |
| | pyrrol-2-yl)-3H-1,4-benzodiazepin-2-amine\|2-Imino-7-butylhexahydro-1H-azepine\|4′- | |
| | Acetamidochalcone | |
| LEPR | 2-Methoxyapomorphine\|3-(1,2-Dihydroacenaphthylen-3-yl)pyridine\|H-Tyr-D-Ala-Gly-Phe-Pro- | Modulator\| |
| | Leu-Trp-NMe-3,5-Bzl(CF3)2\|N-phenyl-1H-pyrazole-3-carboxamide\|5-((2- | Antagonist\| |
| | (Dimethylamino)ethyl)amino)-2-methyl-1,3-benzothiazole-4,7-dione\|4,9-Dihydro-7-methoxy-3H- | Agonist |
| | pyrido(3,4b)indole\|Dopropidil\|[(R)-2-(2-Fluoro-phenyl)-1-methyl-1-(7-ureido-heptylcarbamoyl)- | |
| | ethyl]-carbamic acid (S)-2-methyl-1-phenyl-propyl ester\|(9Z,12E)-12-nitrooctadeca-9,12-dienoic | |
| | acid\|2-Phenylquinoline-8-carboxamide | |
| CSF1R | N-[3-[2-(trifluoromethoxy)phenyl]methylcarbamoyl]phenyl]-2- | Antagonist\| |
| | (trifluoromethyl)benzamide\|Menthol\|4-Hexyl-5-methylbenzene-1,3-diol\|Usistapide\|Csf1R-IN- | Inhibitor\|. |
| | 2\|Simufilam\|Foslinanib\|Tpi-287\|(S)-2-Methyl-1-[(4-methyl-5-isoquinoline)sulfonyl]- | |
| | homopiperazine\|Vimseltinib\|4-((2-(((1R,2R)-2-hydroxycyclohexyl)amino)benzo[d]thiazol-6- | |
| | yl)oxy)-N-methylpicolinamide\|3-(3-Benzylnaphthalen-2-yl)pyridine\|Sulfatinib\|Pexidartinib\|H-DL- | |
| | Tyr(2,6-diMe)-DL-Tic-DL-Lys(Cbz)-NHPh\|2,4″-Dimethoxy-5,3″-dipropyl-biphenyl\|N-[2-(3,4- | |
| | dihydro-2H-quinolin-1-yl)-2-oxoethyl]-2-[3-[(Z)-hydroxyiminomethyl]phenyl]carbamoylamino]-N- | |
| | quinolin-8-ylacetamide\|Tinidazole\|2-(3-Chlorophenyl)histamine\|5-(3-Methoxy-4-((4- | |
| | methoxybenzyl)oxy)benzyl)pyrimidine-2,4-diamine\|4-cyano-N-(5-(1-(2- | |
| | (dimethylamino)acetyl)piperidin-4-yl)-2′,3′,4′,5′-tetrahydro-[1,1′-biphenyl]-2-yl)-1H-imidazole-2- | |
| | carboxamide\|1-(4-((6,7-Dimethoxyquinolin-4-yl)oxy)-2-methoxyphenyl)-3-(1-(thiazol-2- | |
| | yl)ethyl)urea\|3-Phenyl-1H-benzofuro[3,2-c]pyrazole | |
| CEAM3 | 5,6,8-Trimethoxy-4-methylquinolin-2(1H)-one | . |
| FCG2A | #N/A | Antagonist |
| MADCA | Navarixin\|Eremomycin | .\|Inhibitor |
| IL1R2 | 6-((4R)-4-Methyl-1,1-dioxo-1,2,6-thiadiazinan-2-yl)isoquinoline-1-carbonitrile\|2-(4-Methylphenyl)- | Agonist\| |
| | 1H-anthraceno[1,2-d]imidazole-6,11-dione\|#N/A\|N6-methoxy-2-[(4-pyridinyl)ethynyl]adenosine | Modulator\| |
| | | Antagonist\|. |
| ICAM1 | Alicaforsen\|N-(3-cyano-4-methyl-1H-indol-7-yl)-3-cyanobenzene-sulfonamide\|1-(1-Biphenyl-4-yl- | Antagonist\| |
| | 2-phenyl-ethyl)-1H-imidazole\|Naxifylline\|2-((2- | Modulator\|.\| |
| | Phenethylphenoxy)methyl)pyridine\|Selgantolimod\|2-[4-[2-(diethylamino)ethoxy]phenyl]-N-[(2,4- | Inhibitor |
| | difluorophenyl)methyl]-5-fluoropyrimidin-4-amine\|1-(Benzhydrylamino)ethaniminium | |
| | bromide\|Lifitegrast\|L-Leucine,L-alanyl-L-histidyl-L-tryptophyl-L-serylglycyl-L-histidyl-L-cysteinyl- | |
| | L-cysteinyl-\|d[Leu4,Dab8]VP\|2,3-dichloro-N-(3-methoxypyrazin-2- | |
| | yl)benzenesulfonamide\|Haegtftsdvssylegqaakefiawlvkgrg-NH2\|Propionic acid 3-allyl-4-(3- | |
| | isopropylamino-propoxy)-phenyl ester\|H-DArg-Arg-Pro-Hyp-Gly-Igl-Ser-D-BT-OH(JMV1638)\|N- | |
| | [4-(1H-pyrazol-4-yl)-1,3-thiazol-2-yl]pyridin-2-amine\|2-Propen-1-one,1-(4-acetyl-1-piperazinyl)- | |
| | 3-(4-((2-(1-methylethyl)phenyl)thio)-3-nitrophenyl)-\|Dehydropipernonaline\|Pellitorine\|Piperolein | |
| | B\|Pipernonaline\|Procysteine\|Vernakalant | |
| KI3L2 | c[CO-2,3-pyrazine-CO-D-Phe-Arg-Trp-Lys]-NH2 | Antagonist |
| CD80 | Azathioprine\|10-Deazaaminopterin\|4-(4′-Fluorobenzyl)piperidine\|Pyridin-3-ylmethyl-(3,7,11- | .\|Inhibitor\| |
| | trimethyl-dodeca-2,6,10-trienyl)-amine\|Cefetamet Pivoxil HCl\|Phenylalanylmethane\|Tyr-(R)- | CAR-T-Cell- |
| | spiro-Aba-Gly-Phe-NH2[(1R,2S)-2-(methylcarbamothioyl)-2-pyridin-3-ylcyclohexyl] | Therapy\| |
| | benzoate\|Butein | Binder |
| LIRB4 | Oteseconazole | Inhibitor |
| BASI | [[[[(2R,3S,4R,5R)-5-(6-amino-2-propylsulfanylpurin-9-yl)-3,4-dihydroxyoxolan-2-yl]methoxy- | . |
| | hydroxyphosphoryl]oxy-hydroxyphosphoryl]-difluoromethyl]phosphonic acid\|Provena | |
| NECT4 | 8-Methoxy-2-morpholin-4-yl-chromen-4-one\|Deutarserine\|5-(2-((4-(Methylsulfonyl)piperazin-1- | .\|Inhibitor |
| | yl)methyl)-8-morpholinoimidazo[1,2-a]pyrazin-6-yl)pyrimidin-2-amine | |
| AA3R | Tonapofylline\|3-(3-Cyclopentyloxy-4-methoxyphenyl)-3-(1,3-dioxoisoindol-2- | Inhibitor\| |
| | yl)propanamide\|Dmt-Pro-Mmp-Phe-NH2\|NP9Yyc47VZ\|Mcp-neca\|1-(3-Chlorophenyl)-3-[4- | Antagonist\| |
| | (oxolan-2-yl)-11-propyl-3,5,8,10,11-pentazatricyclo[7.3.0.02,6]dodeca-1(12),2,4,7,9-pentaen-7- | Enhancer\| |
| | yl]urea\|3,5,7-Triethoxyflavone\|2-Phenyl-3,5,7-tripropoxychromen-4-one\|3,6-Dichloro-2′- | Agonist |
| | isopropyloxy-4′-methylflavone\|Hemado\|3,6-Dichloro-2-phenyl-4h-chromen-4-one\|3,7-Diethoxy-5- | |
| | hydroxyflavone\|3,5-diethyl 2-methyl-6-phenyl-4-[(E)-2-phenylethenyl]-1,4-dihydropyridine-3,5- | |
| | dicarboxylate\|N-(9-chloro-2-furan-2-yl-[1,2,4]triazolo[1,5-c]quinazolin-5-yl)benzamide\|N-(9- | |
| | chloro-2-furan-2-yl-[1,2,4]triazolo[1,5-c]quinazolin-5-yl)propanamide\|FurceIleran\|Ethyl 4,6- | |
| | diethyl-5-ethylsulfanylcarbonyl-2-phenylpyridine-3-carboxylate\|Ethyl 4-ethyl-5- | |
| | ethylsulfanylcarbonyl-2-phenyl-6-propylpyridine-3-carboxylate\|Propyl 2-(3-chlorophenyl)-4,6- | |
| | diethyl-5-propylsulfanylcarbonylpyridine-3-carboxylate\|Propyl 6-ethyl-5-ethylsulfanylcarbonyl-2- | |
| | phenyl-4-propylpyridine-3-carboxylate\|Sakuranetin\|3-lodo-N(6)-4-aminobenzyladenosine\|\|- | |
| | Abopx\|CID 91827346\|(1S,2R,3S,4R,5S)-4-(6-{[(3-chlorophenyl)methyl]amino}-2-[2-(3,4- | |
| | difluorophenyl)ethynyl]-9H-purin-9-yl)-2,3-dihydroxy-N-methylbicyclo[3.1.0]hexane-1- | |
| | carboxamide\|3,5,7-Trimethoxyflavone\|N6- | |
| | Cyclohexyladenosine\|#N/A\|Peneca\|Visnagin\|Flavone\|2Drz23E4F7\|(8R)-8-ethyl-4-methyl-2- | |
| | phenyl-8,9-dihydro-7H-imidazo[2,1-f]purin-5-one\|CID 11689583\|8-{4-[4-(4- | |
| | chlorophenyl)piperazine-1-sulfonyl]phenyl}-1-propyl-2,3,6,7-tetrahydro-1H-purine-2,6-dione\|1-(2- | |
| | Methoxyphenyl)-3-(2-pyridin-3-ylquinazolin-4-yl)urea\|Xanthine amine congener\|Mpc-meca\|AB- | |
| | Meca\|(2S,3S,4R,5R)-3-amino-5-[6-[5-chloro-2-[(3-methyl-1,2-oxazol-5- | |
| | yl)oxymethyl]phenyl]methylamino]purin-9-yl]-4-hydroxy-N-methyloxolane-2-carboxamide\|8-(3- | |
| | Chlorostyryl)caffeine\|5′-N-Ethylcarboxamidoadenosine\|N-[5-(1-cyclopropyl-2,6-dioxo-3-propyl- | |
| | 7H-purin-8-yl)pyridin-2-yl]-N-methyl-6-(trifluoromethyl)pyridine-3-carboxamide\|8-Ethyl-4-methyl- | |
| | 2-phenyl-3,4,7,8-tetrahydro-5H-imidazo[2,1-i]purin-5-one\|(R,S)-Phpneca\|(2R,3S,4R,5R)-2- | |
| | (hydroxymethyl)-5-[6-[(2S)-1-phenylpropan-2-yl]amino]purin-9-yl]oxolane-3,4-diol\|2- | |
| | Chloroadenosine\|2′-Meccpa\|[125]]AB-Meca\|N6-2-(4-Aminophenyl)ethyladenosine\|2- | |
| | Phenylethylyl-adenosine derivative\|8-Phenyltheophylline\|Serotonin\|9-Ethyl-8-phenylethynyl-9H- | |
| | purin-6-ylamine\|N-(9-chloro-2-(furan-2-yl)-[1,2,4]triazolo[1,5-c]quinazolin-5-yl)-2- | |
| | phenylacetamide\|Reversine\|N*6*-Cyclohexyl-N*2*-ethyl-9H-purine-2,6-diamine\|N*6*-Cyclohexyl- | |
| | N*2*-phenyl-9H-purine-2,6-diamine\|N-Benzyl-N′-cyclohexyl-9H-purine-2,6-diamine\|N*6- | |
| | Cyclooctyl-N*2*-phenyl-9H-purine-2,6-diamine\|Cyclohexyl-(2-phenoxy-9H-purin-6-yl)- | |
| | amine\|Cyclohexyl-(2-phenylsulfanyl-9H-purin-6-yl)-amine\|2-Phenyl-2H-pyrazolo[4,3- | |
| | c]quinoline\|4-Methoxy-2-phenyl-2H-pyrazolo[4,3-c]quinoline\|Nipecotic acid\|6-[(3R)-3- | |
| | aminopiperidin-1-yl]-1-but-2-ynyl-3-[(4-methylquinazolin-2-yl)methyl]pyrimidine-2,4- | |
| | dione\|Methoctramine hydrochloride\|Mesulergine\|[1,2,4]triazolo[1,5-a]quinoxalin-4(5H)-one\|3-(3- | |
| | chloro-1H-pyrazol-5-yl)quinoxalin-2(1H)-one\|2-(1H-benzimidazol-2-yl)quinoxaline\|2-(4-methyl- | |
| | 1H-1,3-benzodiazol-2-yl)quinoxaline\|2-(4-chloro-1H-benzo[d]imidazol-2-yl)quinoxaline\|2-[4- | |
| | (ethylsulfanyl)-1H-benzimidazol-2-yl]quinoxaline\|2-(4-nitro-1H-benzo[d]imidazol-2- | |
| | yl)quinoxaline\|2-(5-chloro-1H-benzo[d]imidazol-2-yl)quinoxaline\|2-(1H-imidazo[4,5-c]pyridin-2- | |
| | yl)quinoxaline\|5-Hydroxy-2-(4-hydroxyphenyl)-7-methoxychroman-4-one\|N6- | |
| | Cyclopentyladenosine\|4′,5,6,7-Tetramethylflavone\|Cirsimaritin\|Butaclamol\|1-Propylxanthine\|1- | |
| | Phenethyl-3,7-dihydro-purine-2,6-dione\|1-Butyl-3,7-dihydro-purine-2,6-dione\|1-Prop-2-ynyl-3,7- | |
| | dihydro-purine-2,6-dione\|1-Cyclopentyl-3,7-dihydro-purine-2,6-dione\|4-methoxy-N-(4-phenyl-1,3- | |
| | thiazol-2-yl)benzamide\|1-Allyl-3,7-dihydro-purine-2,6-dione\|9-Methyl-2-phenyl-5,9-dihydro- | |
| | [1,2,4]triazolo[5,1-a][2,7]naphthyridine-6-carboxylic acid methyl ester\|5-Butyl-8-phenyl-3H- | |
| | [1,2,4]triazolo[5,1-i]purine\|3-Methyl-1-phenethyl-3,7-dihydro-purine-2,6-dione\|1-Benzyl-3-methyl- | |
| | 3,7-dihydro-purine-2,6-dione\|4-methoxy-N-(3-phenyl-1,2,4-thiadiazol-5-yl)benzamide\|3-Isobutyl- | |
| | 1-methylxanthine\|4-Methoxy-N-(3-phenyl-isoquinolin-1-yl)-benzamide\|Galangin\|1-Benzyl-1H- | |
| | 1,3,4b,9-tetraaza-fluorene-2,4-dione\|8-Cyclopentyl-1,3-dipropylxanthine\|(2S,3S,4S)-3- | |
| | (carboxylatomethyl)-4-(prop-1-en-2-yl)pyrrolidine-2-carboxylate\|(2R,3S)-3-(6-amino-9H-purin-9- | |
| | yl)nonan-2-ol\|2-chloro-2″-C-methyl-tecadenoson\|2-Chloro-N6-cyclopentyladenosine\|N6-(3- | |
| | lodobenzyl)-2″-O-methyladenosine\|5,7-dibromo-9H-pyrido[3,4-b]indol-6-ol\|(R)-N-(1-Methyl-2- | |
| | phenylethyl)adenosine\|2-(2′-Indolylethyloxy)Adenosine\|(2R,3R,4S,5R)-2-(2-(2-(1H-pyrrol-3- | |
| | yl)ethoxy)-6-amino-9H-purin-9-yl)-5-(hydroxymethyl)-tetrahydrofuran-3,4-diol\|(2R,3R,4S,5R)-2- | |
| | (2-(2-(1H-indol-3-yl)ethoxy)-6-amino-9H-purin-9-yl)-5-(hydroxymethyl)-tetrahydrofuran-3,4-diol\|2- | |
| | Phenylpropoxyadenosine\|(2R,3R,4S,5R)-2-(6-amino-2-(2-(5-iodo-1H-indol-3-yl)ethoxy)-9H- | |
| | purin-9-yl)-5-(hydroxymethyl)-tetrahydrofuran-3,4-diol\|(2R,3R,4S,5R)-2-(6-amino-2-(2-(5-bromo- | |
| | 1H-indol-3-yl)ethoxy)-9H-purin-9-yl)-5-(hydroxymethyl)-tetrahydrofuran-3,4-diol\|(2R,3R,4S,5R)- | |
| | 2-(6-amino-2-(2-(5-chloro-1H-indol-3-yl)ethoxy)-9H-purin-9-yl)-5-(hydroxymethyl)- | |
| | tetrahydrofuran-3,4-diol\|(2R,3R,4S,5R)-2-(6-amino-2-(2-(5-fluoro-1H-indol-3-yl)ethoxy)-9H-purin- | |
| | 9-yl)-5-(hydroxymethyl)-tetrahydrofuran-3,4-diol\|2-(3″″(7″″-Bromo-indolyl)ethyloxy)adenosine\|2- | |
| | (3″-(4′′-Bromo-indolyl)ethyloxy)adenosine\|(2R,3R,4S,5R)-2-(6-amino-2-(2-(6-bromo-1H-indol- | |
| | 3-yl)ethoxy)-9H-purin-9-yl)-5-(hydroxymethyl)-tetrahydrofuran-3,4-diol\|(2R,3R,4S,5R)-2-(6- | |
| | amino-2-(2-(6-chloro-1H-indol-3-yl)ethoxy)-9H-purin-9-yl)-5-(hydroxymethyl)-tetrahydrofuran-3,4- | |
| | diol\|(2R,3R,4S,5R)-2-[6-amino-2-[2-(1H-indol-3-yl)ethoxy]purin-9-yl]-5-ethyloxolane-3,4- | |
| | diol\|(2R,3S,4R,5R)-5-[6-amino-2-[2-(1H-indol-3-yl)ethoxy]purin-9-yl]-3,4-dihydroxyoxolane-2- | |
| | carboximidamide\|(2R,3R,4S,5R)-2-(6-amino-2-(2-(5-hydroxy-1H-indol-3-yl)ethoxy)-9H-purin-9- | |
| | yl)-5-(hydroxymethyl)-tetrahydrofuran-3,4-diol\|(2R,3R,4S,5R)-2-(6-amino-2-(2-(5-methoxy-1H- | |
| | indol-3-yl)ethoxy)-9H-purin-9-yl)-5-(hydroxymethyl)-tetrahydrofuran-3,4-diol\|2-(3″- | |
| | (Benzoimidazole-1′′-yl)ethyloxy)adenosine\|2-(3″-(Benzotriazole-1″-yl)ethyloxy)adenosine\|N- | |
| | (2,6-diphenylpyrimidin-4-yl)benzamide\|N-(4,6-diphenylpyrimidin-2-yl)propionamide\|N-(4,5- | |
| | diphenylpyrimidin-2-yl)acetamide\|N-(2,6-Diphenylpyrimidin-4-YL)-2-methylbutanamide\|N-(2,6- | |
| | diphenylpyrimidin-4-yl)-3-methylbutyramide\|2-benzyl-2H-pyrazolo[3,4-c]quinolin-4(5H)-one\|2- | |
| | Benzoylaminoquinazoline-4-carboxyanilide\|2-Acetylaminoquinazoline-4-carboxyanilide\|1,2- | |
| | Dihydro-2-oxoquinazoline-4-carboxyanilide\|2-Aminoquinazoline-4-carboxy-(4- | |
| | bromophenyl)amide\|2-Aminoquinazoline-4-carboxyanilide\|N-(2,6-diphenylpyrimidin-4- | |
| | yl)isobutyramide\|Pentanamide,N-(2,6-diphenyl-4-pyrimidinyl)-\|N-(2,6-diphenylpyrimidin-4- | |
| | yl)butyramide\|N-(2,6-diphenylpyrimidin-4-yl)propionamide\|N-(2,6-diphenylpyrimidin-4- | |
| | yl)acetamide\|4-chloro-N-(4,6-diphenylpyrimidin-2-yl)benzamide\|2-cyclohexyl-5,7-di(phenyl)-1H- | |
| | imidazo[4,5-b]pyridine\|2-cyclopentyl-5,7-diphenyl-1H-imidazo[4,5-b]pyridine\|2,6-diphenyl-8- | |
| | tButyl-1-deazapurine\|2,6-Diphenyl-8-(1-ethylpropyl)-1-deazapurine\|2-(2-methylpropyl)-5,7- | |
| | diphenyl-1H-imidazo[4,5-b]pyridine\|5,7-diphenyl-2-propan-2-yl-1H-imidazo[4,5-b]pyridine\|2,6- | |
| | Dphenyl-8-propyl-1-deazapurine\|2,6-Diphenyl-8-ethyl-1-deazapurine\|2,6-Diphenyl-8-methyl-1- | |
| | deazapurine\|2,6-Diphenyl-1-deazapurine\|5,7-diphenyl-3H-imidazo[4,5-b]pyridin-2-ol\|1H-Purine, | |
| | 2,6-diphenyl-\|8-cyclohexyl-2,6-diphenyl-9H-purine\|8-cyclopentyl-2,6-diphenyl-7H-purine\|8- | |
| | isopropyl-2,6-diphenyl-9H-purine\|8-propyl-2,6-diphenyl-9H-purine\|2,6,8-triphenyl-9H-purine\|2-(4- | |
| | methoxyphenyl)-6-phenyl-9H-purine\|2-tolyl-6-phenyl-9H-purine\|2-(4-chlorophenyl)-6-phenyl-9H- | |
| | purine\|2,6-bis(4-tolyl)-9H-purine\|2,6-bis(4-methoxyphenyl)-9H-purine\|2,6-bis(4-chlorophenyl)- | |
| | 9H-purine\|N6-methoxy-2-[(4-methoxyphenyl)ethynyl]adenosine\|2-[(4-acetylphenyl)ethynyl]-N6- | |
| | methoxyadenosine\|2-[(4-fluorophenyl)ethynyl]-N6-methoxyadenosine\|N6-methoxy-2-[(4- | |
| | pentylphenyl)ethynyl]adenosine\|N6-methoxy-2-[(4-methylphenyl)ethynyl]adenosine\|N6-methoxy- | |
| | 2-[(4-pyridinyl)ethynyl]adenosine\|N6-methoxy-2-[(3-pyridinyl)ethynyl]-adenosine\|N6-methoxy-2- | |
| | [(2-pyridinyl)ethynyl]adenosine\|2-(4-hydroxypent-1-yl)-N6-methoxyadenosine\|2-(5-cyano-1-pent- | |
| | 1-ynyl)-N6-methoxyadenosine\|N6-methoxy-2-[2-(trimethylsilyl)ethynyl]adenosine\|2-(hex-1-ynyl)- | |
| | N6-methoxyadenosine\|2-ethynyl-N6-methoxyadenosine\|N6-methoxy-2- | |
| | phenylethynyladenosine\|3-Noradamantyl-1,3-dipropylxanthine\|2-azido-N6-methyl-9-(beta-D- | |
| | ribofuranosyl)adenine\|1-(2-(Diethylamino)quinazolin-4-yl)-3-phenylurea\|1-Phenyl-3-(2-(pyridin-3- | |
| | yl)quinazolin-4-yl)urea\|1-Phenyl-3-(quinazolin-4-yl)urea\|1-Phenyl-3-(2-(pyridin-2-yl)quinazolin-4- | |
| | yl)urea\|1-Phenyl-3-(3-(pyridin-2-yl)isoquinolin-1-yl)urea\|N-(3-pyridin-2-ylisoquinolin-1- | |
| | yl)benzamide\|4-methoxy-N-(3-(pyridin-2-yl)isoquinolin-1-yl)benzamide\|8-Bromo-9-isopropyl-9H- | |
| | adenine\|N-(5-benzoyl-4-phenyl-1,3-thiazol-2-yl)benzamide\|8-Bromo-9-(sec-butyl)-9H-adenine\|8- | |
| | bromo-9-isobutyl-9H-purin-6-amine\|8-Bromo-9-cyclobutyl-9H-adenine\|9-Cyclopentyladenine\|8- | |
| | Bromo-9-cyclopentyl-9H-adenine\|8-Bromo-9-cyclohexyl-9H-adenine\|Ethyl 5-benzoyl-4- | |
| | phenylthiazol-2-ylcarbamate\|2-Ethyl-4-(1,3-thiazol-2-yl)thieno[3,2-d]pyrimidine\|8-Bromo-9-(3- | |
| | hydroxypropyl)-9H-adenine\|2,5″-dichloro-5″-deoxy-N6-cyclopentyladenosine\|N6-((+/−)-endo- | |
| | norborn-2-yl)adenosine\|2-phenyl-2H-pyrazolo[4,3-d]pyrimidin-7(6H)-one\|6-Hydroxy-5,7-dimethyl- | |
| | beta-carboline\|8-Hydroxy-5,7,9-trimethyl-delta-carboline\|8-Hydroxy-7,9-dimethyl-delta- | |
| | carboline\|N-(2-(furan-2-yl)-3,4″-bipyridin-6-yl)acetamide\|N-[2-[2-phenyl-6-[4-(3- | |
| | phenylpropyl)piperazine-1-carbonyl]-7H-pyrrolo[3,2-e]pyrimidin-4-yl]amino]ethyl]acetamide\|N-(2- | |
| | hydroxyethyl)-2-(4-(1-methyl-2,4-dioxo-3-propyl-2,3,4,5-tetrahydro-1H-pyrrolo[3,2-d]pyrimidin-6- | |
| | yl)phenoxy)acetamide\|4-(4-Butylpiperidin-1-yl)-1-(2-methylphenyl)butan-1-one\|4-Ethoxy-7-((E)- | |
| | styryl)-furo[3,2-g]chromen-5-one\|Dizocilpine\|5′-Deoxy-5′-methylthioadenosine\|2-({6-Amino-3,5- | |
| | dicyano-4-[4-(cyclopropylmethoxy)phenyl]pyridin-2-yl}sulfanyl)acetamide\|(1S,2R,3S,4R,5S)-4-[2- | |
| | chloro-6-[(3-chlorophenyl)methylamino]purin-9-yl]-2,3-dihydroxy-N-methylbicyclo[3.1.0]hexane- | |
| | 1-carboxamide\|Cyclohexanecarboxamide,N-(2-((3,4-dichlorophenyl)amino)-4-quinolinyl)- | |
| | \|Chloro-IB-MECA\|Cipargamin\|IB-Meca\|Metrifudil\|Bemesetron\|2-(Furan-2-yl)-7-(3-(4- | |
| | methoxyphenyl)propyl)-7H-pyrazolo[4,3-e][1,2,4]triazolo[1,5-c]pyrimidin-5-amine | |
| TACT | 3-[5-Chloranyl-6-[(1~{r})-1-(6-Methylpyridazin-3-YI)ethoxy]-1,2-Benzoxazol-3-YI]propanoic Acid | Antagonist |
| CD22 | (S)-2-((R)-8-(2,4-dichlorophenyl)-2-hydroxyoctyl)-2-hydroxysuccinic acid\|Napsagatran\|2-(4- | CAR-T-Cell- |
| | (Methylsulfonyl)phenyl)-3-phenylquinoline\|3-[5-Chloranyl-6-[(1~{r})-1-(6-Methylpyridazin-3- | Therapy(Dual |
| | Yl)ethoxy]-1,2-Benzoxazol-3-YI]propanoic Acid\|4,5-Bis[4-methylanilino]phthalimide\|N′-hydroxy- | specific)\|.\| |
| | N-[(3-phenylphenyl)methyl]propanediamide\|2,4,4-Triphenylimidazoline\|c[Nle-Val-D-Nal(2″)-Arg- | Modulator\| |
| | Trp-Glu]-NH2\|Cilobradine\|c[Glu22-Orn26][Gly8]GLP-1(7-37)-NH2\|8-(4-bromophenyl)-N-hydroxy- | CAR-T-Cell- |
| | 8-oxooctanamide\|Chromeno[2,3-d]pyrimidine-2,4-dione\|Org-24598 (lithium)\|2- | Therapy\| |
| | Benzoylaminoquinazoline-4-carboxyanilide\|1-(4-Nitrophenyl)-2-phenylethane-1,2- | Immunomodulator\| |
| | dione\|Oxymorphone\|1-Butyl-3-methyl-3,7-dihydro-purine-2,6-dione\|4-[1-(1-Naphthyl)propyl]-1H- | Inhibitor\| |
| | imidazole\|1-(2-Amino-2-cyclohexylacetyl)pyrrolidine-2-carbonitrile\|2-(2,4-Dichlorophenoxy)-5-(3- | Binder |
| | phenylpropyl)phenol\|Ablukast\|4-(4-Chlorophenylthio)-2-(pyridin-2- | |
| | yl)pyrimidine\|Sibofimloc\|Phosphinic acid,P,P-bis(1-aziridinyl)-,(1R)-1-(3-(3- | |
| | ((dimethylamino)carbonyl)phenoxy)-4-nitrophenyl)ethyl ester\|Valemetostat\|1,4,5,6- | |
| | Tetrahydropyrimidin-2-amine\|Paeoniflorin\|n-Cyano-1-[(3,4-dimethoxyphenyl)methyl]-3,4-dihydro- | |
| | 6,7-dimethoxy-n′-octyl-2(1h)-isoquinoline-carboximidamide\|4-Methyl-5-(2-nitroxyethyl) thiazole | |
| | HCI\|((4-(4-Amidinophenoxy)butanoyl)aspartyl)valine\|6-{2-[1-Benzyl-5-(4-fluoro-phenyl)-3- | |
| | isopropyl-1H-pyrazol-4-yl]-ethyl}-4-hydroxy-tetrahydro-pyran-2-one\|Testosterone buciclate\|(R,S)- | |
| | 1-(5-methyl-1H-indol-1-yl)propan-2-amine\|(12E,20Z,18S)-8-hydroxyvariabilin\|isorhamnetin 3-O- | |
| | rhamnoside\|(S)-N2-{4-[(3-fluorobenzyl)oxy]benzyl}serinamide\|3-lodo-N(6)-4- | |
| | aminobenzyladenosine\|2H-Pyran-2-one,6-((1E)-4,4-bis(4-fluorophenyl)-3-(1-methyl-1H-tetrazol- | |
| | 5-yl)-1,3-butadien-1-yl)tetrahydro-4-hydroxy-,(4R,6S)-rel-\|Oxytocin\|2-(1,3-benzodioxol-5-yl)-3H- | |
| | naphtho[3,2-e]benzimidazole-6,11-dione\|1-Amino-6-cyclohex-3-enylmethyloxypurine\|(E)-N-[6- | |
| | [(6-chloro-1,2,3,4-tetrahydroacridin-9-yl)amino]hexyl]-3-(3,4-dihydroxyphenyl)prop-2- | |
| | enamide\|Pentagalloylglucose\|5-((3,5,5,8,8-pentamethyl-5,6,7,8-tetrahydronaphthalen-2- | |
| | yl)methyl)-N-(2,4,6-trimethoxyphenyl)furan-2-carboxamide | |
| CD33 | Neurolite\|N-[(4-carbamimidoylphenyl)methyl]-3-(2-methyl-4- | .\|CAR-T-Cell- |
| | sulfamoylphenyl)benzamide\|Phenytoin\|Luseogliflozin\|Desmosterol\|Benzoic acid,p-hydroxy-,(p- | Therapy\| |
| | (diethylamino)benzylidene)hydrazide\|Dezocine\|4-Amino-2-deoxy-2,3-dehydro-n-neuraminic | Modulator\| |
| | acid\|[5-(2-Chlorophenyl)-1-pentylpyrrol-3-yl]-naphthalen-1-ylmethanone\|8-isopropyl-2,6- | Inhibitor\| |
| | diphenyl-9H-purine\|Actinium-225\|Compstatin 40\|Methyl N-({(2s,3s)-3- | Antagonist |
| | [(Propylamino)carbonyl]oxiran-2-YI}carbonyl)-L-Isoleucyl-L-Prolinate\|Vorolanib\|3- | |
| | (Phenylsulfinyl)-L-alanine\|Valemetostat\|Phosphinic acid,P,P-bis(1-aziridinyl)-,(1R)-1-(3-(3- | |
| | ((dimethylamino)carbonyl)phenoxy)-4-nitrophenyl)ethyl ester\|N-(1-((2- | |
| | (Dimethylamino)ethyl)amino)-2-methyl-1-oxopropan-2-yl)-4-(4-(2-methyl-5-((2S,3R,4R,5S,6R)- | |
| | 3,4,5-trihydroxy-6-(methylthio)tetrahydro-2H-pyran-2-yl)benzyl)phenyl)- | |
| | butanamide\|Emicerfont\|methyl(E)-2-[2-[4-[(E)-C-ethyl-N-propoxycarbonimidoyl]-2,5- | |
| | dimethylphenoxy]methyl]phenyl]-3-methoxyprop-2-enoate\|4-[3-(2-Fluorophenoxy)phenyl]-1- | |
| | phosphonatobutane-1-sulfonate\|(6aR,8S,9S,10aR)-9-(hydroxymethyl)-6,6-dimethyl-3-(2- | |
| | methyloctan-2-yl)-8,9-ditritio-7,8,10,10a-tetrahydro-6aH-benzo[c]chromen-1- | |
| | ol\|Tetrahydroquinoline A\|2-[(5-Phenyl-1,3-oxazol-2-yl)amino]benzonitrile\|Vadastuximab | |
| | talirine\|Formic Acid | |
| LAIR1 | (1S,3aR,6aS)-2-((R)-2-((S)-2-cyclohexyl-2-(pyrazine-2-carboxamido)acetamido)-3,3- | Inhibitor |
| | dimethylbutanoyl)-N-((S)-1-(cyclopropylamino)-1,2-dioxohexan-3- | |
| | yl)octahydrocyclopenta[c]pyrrole-1-carboxamide | |
| TIGIT | 1,2-(BiS(1,3-dithia-2-bismolane)thio)ethane\|Tame\|Dextilidine\|1-(4-fluorophenyl)-N-[3-fluoro-4- | Inhibitor\| |
| | [(3-phenyl-1H-pyrrolo[2,3-b]pyridin-4-yl)oxy]phenyl]-2,3-dimethyl-5-oxopyrazole-4- | Antagonist\|. |
| | carboxamide\|N-Phenylethylenediamine\|2-(2-Amino-3-methoxyphenyl)-4H-1-benzopyran-4-one | |
| FGFR4 | Ascrolimus\|7-(4-fluoroanilino)-1-[(1R,3R)-3-hydroxycyclopentyl]-3-(4-methoxyphenyl)-4H- | .\|Inhibitor\| |
| | pyrimido[4,5-d]pyrimidin-2-one\|8-Bicyclo[2.2.1]hept-2-yl-2-{4-[4-(3-hydroxypropyl)piperidin-1- | Antagonist |
| | yllphenylamino}-8H-pyrido[2,3-d]pyrimidin-7-one\|H3B- | |
| | 6527\|Fisogatinib\|Roblitinib\|Trimeprazine\|Actb-1003\|Des-AA1,2,5-[D-Trp8,IAmp9,Tyr11]Cbm- | |
| | SRIF | |
| MUSK | (S)-5-fluoro-2-(1-(5-fluoropyridin-2-yl)ethylamino)-6-(5-methyl-1H-pyrazol-3- | Inhibitor |
| | ylamino)nicotinonitrile\|7-Chloro-3-oxo-8-(1,3-thiazol-5-ylmethylamino)-11,16-dioxa-2,4,19,21- | |
| | tetrazatricyclo[15.3.1.05,10]henicosa-1(20),5,7,9,17(21),18-hexaene-18-carbonitrile | |
| CEAM6 | Piperlongumine | Blocker |
| CD276 | 6-(benzylamino)-9-butyl-9H-purine-2-carbonitrile\|Pirotiodecane\|Tarenflurbil\|[9-[6-[[4-[2-[4-[3-(2- | .\|Modulator\| |
| | ethoxyphenyl)-4-oxoquinazolin-2-yl]methyl]piperazin-1-yl]-2- | Immunomodulator\| |
| | oxoethoxy]phenyl]methylamino]hexyl]-9-azabicyclo[3.3.1]nonan-3-yl]N-(2-methoxy-5- | Inhibitor |
| | methylphenyl)carbamate\|Evenamide\|BAY 598-Bio-X\|7-Methoxy-1,2,3,4-tetrahydroacridin-9- | |
| | amine\|6-Formamidopenicillanic acid\|Belvarafenib\|Penoxsulam | |
| FCAR | (1S,4R)-4-hydroxy-2,2-dimethyl-4-[5-(3-methyl-5-{[4-(trifluoromethyl)pyrimidin-2- | |
| | ylamino}phenyl)-1,3-thiazol-2-yl]cyclohexanecarboxylic acid | |
| SHPS1 | Maralixibat\|N~2~-hexyl-6,7-dimethoxy-N~4~-(1-methylpiperidin-4-yl)quinazoline-2,4- | Antagonist\|. |
| | diamine\|Penoxsulam | |
| FGFR1 | Derazantinib\|[D-Ncy(isopropyl)7]acyline\|2-[3-(4-Phenylphenyl)propanamido]benzoic acid\|Actb- | Inhibitor\|.\| |
| | 1003\|4-(4-ethylpiperazin-1-yl)-N-[6-(2-fluoro-3-methoxyphenyl)-1H-indazol-3-yl]benzamide\|3-[(4- | Agonist\| |
| | chlorophenyl)-(1H-imidazol-2-yl)methylidene]-5-[(1-ethylpiperidin-4-yl)amino]-1H-indol-2-one\|N- | Modulator\| |
| | [(1S)-1-(5-fluoropyridin-2-yl)ethyl]-3-(3-propan-2-yloxy-1H-pyrazol-5-yl)imidazo[4,5-b]pyridin-5- | Binder\| |
| | amine\|3-(3-methoxybenzyl)-1H-pyrrolo[2,3-b]pyridine\|#N/A\|2-(Pyridin-4-Ylmethylamino)-~{n}-[3- | Antagonist |
| | (Trifluoromethyl)phenyl]benzamide\|6-(2,6-dichlorophenyl)-8-methyl-2-((3- | |
| | (methylthio)phenyl)amino)pyrido[2,3-d]pyrimidin-7(8H)-one\|3-(1H-Benzimidazol-2-YL)-1H- | |
| | indazole\|7-(4-fluoroanilino)-1-[(1R,3R)-3-hydroxycyclopentyl]-3-(4-methoxyphenyl)-4H- | |
| | pyrimido[4,5-d]pyrimidin-2-one\|6-(2,6-Dichlorophenyl)-2-{[3-(hydroxymethyl)phenyl]amino}-8- | |
| | methylpyrido[2,3-D]pyrimidin-7(8H)-one\|6-(2,6-Dichlorophenyl)-8-methyl-2-(4-morpholin-4- | |
| | ylanilino)pyrido[2,3-d]pyrimidin-7-one\|Pyrido(2,3-d)pyrimidin-7(8H)-one,6-(2,6-dichlorophenyl)- | |
| | 2-((4-fluorophenyl)amino)-8-methyl-\|2-[(4-Ethoxyphenyl)amino]-8-methyl-6-(2,6- | |
| | dichlorophenyl)pyrido[2,3-d]pyrimidine-7(8H)-one\|2-[4-[2- | |
| | [Ethyl(acetyl)amino]ethoxy]phenyl]amino]-8-methyl-6-(2,6-dichlorophenyl)pyrido[2,3- | |
| | d]pyrimidine-7(8H)-one\|6-(2,6-Dichlorophenyl)-2-[(4-Fluoro-3-Methylphenyl)amino]-8- | |
| | Methylpyrido[2,3-D]pyrimidin-7(8h)-One\|3-[5-[(Z)-[5-(anilinocarbamoyl)-2-oxo-1H-indol-3- | |
| | ylidene]methyl]-2,4-dimethyl-1H-pyrrol-3-yl]propanoic acid\|8-Bicyclo[2.2.1]hept-2-yl-2-{4-[4-(3- | |
| | hydroxypropyl)piperidin-1-yl]phenylamino}-8H-pyrido[2,3-d]pyrimidin-7-one\|2(1H)-Pyridinone,4- | |
| | ((2-(4-chloro-1H-pyrazol-1-yl)ethyl)amino)-3-(5-(1-(3-fluoropropyl)-4-piperidinyl)-7-methyl-1H- | |
| | benzimidazol-2-yl)-\|(E)-(4-(2-(1H-indazol-3-yl)vinyl)phenyl)(piperazin-1- | |
| | yl)methanone\|Martinostat\|MK-2461\|Debio-1347\|2-Amino-2-(3-chlorophenyl)acetic acid\|3-(3- | |
| | Cyclopentyloxy-4-methoxyphenyl)-3-(1,3-dioxoisoindol-2-yl)propanamide\|Valerenic acid | |
| | amide\|Azd-4547\|Infigratinib\|Sulfatinib\|Lucitanib\|Orantinib\|Pemigatinib\|Semaxanib\|Nintedanib | |
| I11RA | 9-methyl-2,3,4,9-tetrahydro-1H-pyrido[3,4-b]indole\|1-Fluoro-5-phenyl-3-aza- | Modulator\| |
| | bicyclo[3.1.0]hexane\|cyclo[RGDfK(cypate)] | Binder |
| SEM4D | 1-(1-Propionylpiperidin-4-yl)-3-m-tolylurea\|Onametostat | .\|Inhibitor |
| CD86 | 4-(4′-Fluorobenzyl)piperidine\|Pyridin-3-ylmethyl-(3,7,11-trimethyl-dodeca-2,6,10-trienyl)- | .\|Inhibitor\| |
| | amine\|Butein\|5-(Aminomethyl)naphthalen-2-ol\|3-(1,1′-Biphenyl-4-yl)quinuclidine\|N-[(3-chloro-4- | Binder |
| | fluorophenyl)methyl]-1-methyl-N-[[(1R,5S)-3-methyl-3-azabicyclo[3.1.0]hexan-6- | |
| | yl]methyl]imidazole-4-carboxamide\|Fasiglifam | |
| NTRK3 | 4-cyano-N-(5-(1-(2-(dimethylamino)acetyl)piperidin-4-yl)-2′,3′,4′,5′-tetrahydro-[1,1′-biphenyl]-2- | Inhibitor\| |
| | yl)-1H-imidazole-2-carboxamide\|(Z)-1-(3-((3-((1H-pyrrol-2-yl)methylene)-2-oxoindolin-6- | Modulator |
| | yl)amino)-4-methylphenyl)-3-(2-fluoro-5-(trifluoromethyl)phenyl)urea\|(S)-5-fluoro-2-(1-(5- | |
| | fluoropyridin-2-yl)ethylamino)-6-(5-methyl-1H-pyrazol-3-ylamino)nicotinonitrile\|5-chloro-N2-(1-(5- | |
| | fluoropyridin-2-yl)ethyl)-N4-(5-isopropoxy-1H-pyrazol-3-yl)pyrimidine-2,4-diamine\|N-[(1S)-1-(5- | |
| | fluoropyridin-2-yl)ethyl]-3-(3-propan-2-yloxy-1H-pyrazol-5-yl)imidazo[4,5-b]pyridin-5-amine\|3- | |
| | benzyl-N-(2-morpholinoethyl)quinoxalin-2-amine\|5-[4-[2-(4-oxo-2H-1,3-benzoxazin-3- | |
| | yl)ethoxy]phenyl]methyl]-1,3-thiazolidine-2,4-dione\|(3S,4S,5S)-5-(Hydroxymethyl)piperidine-3,4- | |
| | diol\|2-[6-(4-Chlorophenoxy)hexyl]-1-cyano-3-pyridin-4-ylguanidine\|3-amino-6-methyl-N-pyridin-2- | |
| | ylpyrazine-2-carboxamide\|Aekkdegpyrmehfrwgsppkd\|Altiratinib\|MK- | |
| | 2461\|Entrectinib\|Larotrectinib\|N-(6-fluoro-3-pyridyl)-1-[4-[(5-isopropyl-1H-pyrazol-3-yl)amino]- | |
| | 6,7-dihydro-5H-cyclopenta[d]pyrimidin-2-yl]pyrrolidine-2-carboxamide\|4-[3-amino-4-(oxan-4- | |
| | yloxy)-1H-indazol-6-yl]-2-(4-cyclohexylpiperazin-1-yl)-N-methylbenzamide\|4-[3-amino-4-(1,1,1- | |
| | trifluoropropan-2-yloxy)-1H-indazol-6-yl]-2-(4-phenylpiperazin-1-yl)benzamide\|4-[3-amino-4- | |
| | (oxan-4-yloxy)-1H-indazol-6-yl]-2-(4-phenylpiperazin-1-yl)benzamide\|XX8E5WP7FS | |
| KI2L3 | CID 16750271\|Nelonicline\|2-thioUTP | Antagonist\|.\| |
| | | Modulator |
| PGFRA | PDGF Receptor Tyrosine Kinase Inhibitor II\|1-cyclopentyl-3-(1H-pyrrolo[2,3-b]pyridin-5-yl)-1H- | Inhibitor\|.\| |
| | pyrazolo[3,4-d]pyrimidin-4-amine\|5-((5-(4-((4-(2-hydroxyethyl)piperazin-1-yl)methyl)-3- | Modulator |
| | (trifluoromethyl)phenylcarbamoyl)-2-methylphenyl)ethynyl)-N,1-dimethyl-1H-imidazole-2- | |
| | carboxamide\|3-[(4-chlorophenyl)-(1H-imidazol-2-yl)methylidene]-5-[(1-ethylpiperidin-4-yl)amino]- | |
| | 1H-indol-2-one\|1-(2-(5-(2-Methoxyethoxy)benzimidazol-1-yl)quinolin-8-yl)piperidin-4-ylamine\|4- | |
| | [[(2S)-2-(3-Chlorophenyl)-2-hydroxyethyl]amino]-3-[7-methyl-5-(4-morpholinyl)-1H-benzimidazol- | |
| | 2-yl]-2(1H)-pyridinone\|5,11-dimethyl-6H-pyrido[4,3-b]carbazol-9-ol\|N-(3-fluorophenyl)-6,7- | |
| | dimethoxy-1,4-dihydroindeno[1,2-c]pyrazol-3-amine\|5-[(Z)-(5-chloro-2-oxo-1,2-dihydro-3H-indol- | |
| | 3-ylidene)methyl]-N-[2-(diethylamino)ethyl]-2,4-dimethyl-1H-pyrrole-3-carboxamide\|6,7- | |
| | Dimethoxy-4-(3,4-dimethoxyphenoxy)quinoline\|6,7-dimethoxy-N-methyl-N-phenylquinazolin-4- | |
| | amine; hydrochloride\|6,7-Dimethoxy-3-pyridin-3-yl-quinoline\|4-(6,7-Dimethoxy-quinolin-3-yl)- | |
| | benzoic acid\|6,7-Dimethoxy-3-(4-methoxy-phenyl)-quinoline\|7-Fluoro-3-thiophen-3-yl- | |
| | quinoline\|6,7-Dimethoxy-3-thiophen-2-yl-quinoline\|7-Methoxy-3-thiophen-3-yl-quinoline\|5-Fluoro- | |
| | 3-thiophen-3-yl-quinoline\|3-(3,4-Dimethoxy-phenyl)-6,7-dimethoxy-quinoline\|4-(6,7- | |
| | Dimethoxyquinolin-3-yl)phenol\|7-Thiophen-3-yl-[1,3]dioxolo[4,5-g]quinoline\|6,7-Difluoro-3- | |
| | thiophen-3-yl-quinoline\|6,7-Dimethoxy-3-p-tolyl-quinoline\|6,7-Dimethoxy-3-(3-methoxy-phenyl)- | |
| | quinoline\|6,7-Dimethoxy-3-phenylethynyl-quinoline\|3-Cyclopentyl-6,7-dimethoxy-quinoline\|6- | |
| | Methoxy-3-thiophen-3-yl-quinoline\|3-(2-Cyclohexyl-ethyl)-6,7-dimethoxy-quinoline\|7-Methoxy-3- | |
| | pyridin-4-yl-quinoline\|6,7-Dimethoxy-3-((E)-styryl)-quinoline\|3-Pyridin-3-yl-quinoline-6,7- | |
| | diol\|Benzyl-(6,7-dimethoxy-quinolin-3-yl)-amine\|5,7-Dimethoxy-3-thiophen-3-yl-quinoline\|3- | |
| | Cyclopent-1-enyl-6,7-dimethoxy-quinoline\|3-(3-Fluoro-phenyl)-6,7-dimethoxy-quinoline\|3-(4- | |
| | Pyridinyl)quinoline\|5,7-Dimethyl-3-thiophen-3-yl-quinoline\|6,7-Dimethoxy-3-(2-methoxy-phenyl)- | |
| | quinoline\|3-Benzyloxy-6,7-dimethoxy-quinoline\|6,7-Dimethoxy-3-phenyl-quinoline\|6,7- | |
| | Dimethoxy-3-thiophen-3-yl-quinoline\|5-(6,7-Dimethoxy-quinolin-3-yl)-1H-pyridin-2-one\|3-(3,4- | |
| | Difluoro-phenyl)-6,7-dimethoxy-quinoline\|3-(4-Fluoro-phenyl)-6,7-dimethoxy-quinoline\|3-(3,4- | |
| | Dichloro-phenyl)-6,7-dimethoxy-quinoline\|3-(3-Thienyl)quinoline\|6-Methoxy-3-pyridin-4-yl- | |
| | quinoline\|7-Chloro-3-pyridin-4-yl-quinoline\|5,7-Dimethoxy-3-(4-pyridinyl)quinoline\|5,6,7- | |
| | Trimethoxy-3-pyridin-4-yl-quinoline\|3-((E)-Styryl)-quinoline\|3-(1H-indol-3-yl)-6,7-dimethoxy- | |
| | quinoline\|3-(1H-indol-3-yl)quinoline\|3-Cyclohexylethynyl-6,7-dimethoxy-quinoline\|6,7-Dichloro-3- | |
| | thiophen-3-yl-quinoline\|6,7-Dimethoxy-3-(4-nitro-phenyl)-quinoline\|3-Pyridin-4-yl-quinoline-5,7- | |
| | diol\|6,7-Dimethoxy-3-pyridin-4-yl-quinoline\|3-Pyridin-4-yl-quinolin-7-ol\|6-(2,6-dichlorophenyl)-8- | |
| | methyl-2-((3-(methylthio)phenyl)amino)pyrido[2,3-d]pyrimidin-7(8H)-one\|FIt-3 Inhibitor II\|(5- | |
| | fluoro-1H-indol-2-yl)-(1H-indol-2-yl)methanone\|Bis(1H-2-indolyl)methanone 12\|(1H-indol-2-yl)(5- | |
| | phenoxy-1H-indol-2-yl)methanone\|Bis(1H-2-indolyl)methanone 11\|(5-hydroxy-1H-indol-2-yl)-(1H- | |
| | indol-2-yl)methanone\|di(1H-indol-2-yl)methanone\|7-(4-fluoroanilino)-1-[(1R,3R)-3- | |
| | hydroxycyclopentyl]-3-(4-methoxyphenyl)-4H-pyrimido[4,5-d]pyrimidin-2-one\|6-(2,6- | |
| | Dichlorophenyl)-2-{[3-(hydroxymethyl)phenyl]amino}-8-methylpyrido[2,3-D]pyrimidin-7(8H)- | |
| | one\|6-(2,6-Dichlorophenyl)-8-methyl-2-(4-morpholin-4-ylanilino)pyrido[2,3-d]pyrimidin-7- | |
| | one\|Pyrido(2,3-d)pyrimidin-7(8H)-one,6-(2,6-dichlorophenyl)-2-((4-fluorophenyl)amino)-8- | |
| | methyl-\|2-[(4-Ethoxyphenyl)amino]-8-methyl-6-(2,6-dichlorophenyl)pyrido[2,3-d]pyrimidine- | |
| | 7(8H)-one\|2-[4-[2-[Ethyl(acetyl)amino]ethoxy]phenyl]amino]-8-methyl-6-(2,6- | |
| | dichlorophenyl)pyrido[2,3-d]pyrimidine-7(8H)-one\|6-(2,6-Dichlorophenyl)-2-[(4-Fluoro-3- | |
| | Methylphenyl)amino]-8-Methylpyrido[2,3-D]pyrimidin-7(8h)-One\|4-(1H-benzimidazol-1- | |
| | yl)aniline\|1H-Benzimidazole,5-methoxy-1-phenyl-\|1-Phenyl-5-hydroxybenzimidazole\|5- | |
| | Substituted 1-Phenylbenzimidazole 22\|1-Phenyl-1H-benzoimidazole\|3-benzyl-N-(2- | |
| | morpholinoethyl) quinoxalin-2-amine\|[2-(Octadecoxymethyl)oxolan-2-yl]methyl 2- | |
| | (trimethylazaniumyl)ethyl phosphate\|Amcasertib\|3-amino-6-methyl-N-pyridin-2-ylpyrazine-2- | |
| | carboxamide\|Amuvatinib\|5- | |
| | Methoxytryptoline\|Famitinib\|Crenolanib\|Lucitanib\|Avapritinib\|Ripretinib\|Semaxanib\|2- | |
| | (Aminomethyl)-5-(2″-naphthyl)tetrahydrofuran\|(Z)-3-(3,4-Dimethoxy-phenyl)-2-pyridin-3-yl- | |
| | acrylonitrile\|6,7-Dimethyl-2-phenylquinoxaline\|H-DL-Tyr(2,6-diMe)-DL-Tic-DL-Lys(Cbz)- | |
| | NHPh\|2,4″-Dimethoxy-5,3″-dipropyl-biphenyl\|N-[2-(3,4-dihydro-2H-quinolin-1-yl)-2-oxoethyl]-2- | |
| | [[3-[(Z)-hydroxyiminomethyl]phenyl]carbamoylamino]-N-quinolin-8-ylacetamide\|Tinidazole\|2-(3- | |
| | Chlorophenyl)histamine | |
| MERTK | N-[4-(6,7-dimethoxyquinolin-4-yl)oxy-3-fluorophenyl]-4-ethoxy-1-(4-fluoro-2- | Inhibitor |
| | methylphenyl)pyrazole-3-carboxamide\|8-Bromo-2-((1-methylpiperidin-4-yl)amino)-4-((4- | |
| | phenoxyphenyl)amino)pyrido[4,3-D]pyrimidin-5(6H)-one\|1-[(Trans-4-Aminocyclohexyl)methyl]-N- | |
| | Butyl-3-(4-Fluorophenyl)-1h-Pyrazolo[3,4-D]pyrimidin-6-Amine\|(1r,4r)-4-(2-(butylamino)-5-(4-((4- | |
| | methylpiperazin-1-yl)methyl)phenyl)-7H-pyrrolo[2,3-d]pyrimidin-7- | |
| | yl)cyclohexanol\|cyclo[Cys6,Cys10]N/OFQ(1-13)NH2\|N-[4-(6,7-dimethoxyquinolin-4-yl)oxy-3- | |
| | fluorophenyl]-3-(4-fluorophenyl)-2,4-dioxo-1-propan-2-ylpyrimidine-5- | |
| | carboxamide\|2MT30Ehi63\|(1s,2s)-2-(3,4-Dichlorobenzoyl)cyclopropanecarboxylic Acid\|(2S)-2- | |
| | [(2S)-2-[(2S)-2-[(2S,3R)-2-[(2S)-2-[(2S)-2-[(2S)-2-[(2S)-2-[(2S)-2-[(2- | |
| | acetamidoacetyl)amino]propanoyl]amino]-5-amino-5-oxopentanoyl]amino]-3- | |
| | phenylpropanoyl]amino]-3-hydroxypropanoyl]amino]-6-aminohexanoyl]amino]-3- | |
| | hydroxybutanoyl]amino]propanoyl]amino]propanoyl]amino]-6-aminohexanoic acid\|MK-2461\|N- | |
| | (5-((6,7-Dimethoxyquinolin-4-yl)oxy)pyridin-2-yl)-2,5-dioxo-1-phenyl-1,2,5,6,7,8- | |
| | hexahydroquinoline-3-carboxamide | |
| CD19 | 2-amino-N-[(4-chlorophenyl)methyl]-N′-hydroxypropanediamide\|2-Amino-3-(3-hydroxy-5-tert- | CAR-T-Cell- |
| | butylisoxazol-4-yl)propionic acid\|(S)-1-(2-Amino-2-carboxyethyl)-3-(2-carboxybenzyl)pyrimidine- | Therapy(Dual |
| | 2,4-dione\|2-[3-[2-[2-(Methyl-phenylamino)-2-oxoethyl]-phenylamino]-2- | specific)\| |
| | oxoethyl]carbamoylamino]phenyl]acetic acid\|10-(4-morpholinylcarbonyl)-10H-phenothiazine\|6- | CAR-T-Cell- |
| | Chloro-2-piperazin-1-yl-quinoline\|cyclo(-D-Tyr-L-Arg-L-Arg-L-Nal-L-Ala-)\|1-[1,3]Oxazolo[4,5- | Therapy\|.\| |
| | b]pyridin-2-yloctan-1-one\|cis-Montelukast\|1H-Pyrrolo(1,2-a)indole-1-acetic acid, 9-((4- | Inhibitor\| |
| | chlorophenyl)thio)-6-fluoro-2,3-dihydro-,(1S)-\|Tecovirimat\|4-trans-2-Carboxy-5,7-dichloro-4- | Modulator\| |
| | phenylaminocarbonylamino-1,2,3,4-tetrahydroquinoline\|(-)-3-O-acetylspectaline\|(2S)-2-Amino-4- | Immunostimulant |
| | (2-amino-4-chlorophenyl)-4-oxobutanoic acid\|2-Bromo-N-[(2s,3s)-4-{[2-(2,4- | |
| | Dichlorophenyl) ethyl][3-(1,3-Dioxo-1,3-Dihydro-2h-Isoindol-2-YI)propanoyl]amino}-3-Hydroxy-1- | |
| | (3-Phenoxyphenyl)butan-2-YI]-4,5-Dimethoxybenzamide\|Ethyl 6-(2-morpholin-4-ylethyl)pyridine- | |
| | 3-carboxylate\|Cbz-Ile-Leu-Ala-LeuVSMe\|Amino-thiophen-2-YL-acetic acid\|Hdac inhibitor CHR- | |
| | 3996\|Triphenylbutanamine\|Zandelisib\|2-(4-(Methylsulfonyl)phenyl)-3-phenylquinoline\|1,4- | |
| | Diaminoanthraquinone\|(S,E)-N-(1-((5-(2-((4-Cyanophenyl)amino)-4-(propylamino)pyrimidin-5- | |
| | yl)pent-4-yn-1-yl)amino)-1-oxopropan-2-yl)-4-(dimethylamino)-N-methylbut-2-enamide\|3-[5- | |
| | Chloranyl-6-[(1~{r})-1-(6-Methylpyridazin-3-YI)ethoxy]-1,2-Benzoxazol-3-Yl]propanoic Acid\|(E)-2- | |
| | (2-(quinolin-2-yl)vinyl)phenol\|methyl (2S,3S,5R)-8-methyl-3-phenyl-8-azabicyclo[3.2.1]octane-2- | |
| | carboxylate\|5-chloro-4-N-(cyclopropylmethyl)-2-methyl-4-N-propyl-6-N-(2,4,6- | |
| | trichlorophenyl)pyrimidine-4,6-diamine\|c[Nle-Val-D-Nal(2″)-Arg-Trp-Glu]-NH2\|N-{3-[4-(4- | |
| | Phenylthiophen-2-YI)phenyl]propanoyl}-L-Alpha-Glutamyl-L-Alpha-Glutamyl-Amide\|c[Glu22- | |
| | Orn26][Gly8]GLP-1(7-37)-NH2\|2-[(4-Methoxyphenyl)-diphenylmethoxy]ethanol\|N-[3-(3- | |
| | methoxyphenyl)propyl]acetamide\|5,6,8-Trimethoxy-4-methylquinolin-2(1H)-one\|1-(2-(2- | |
| | Ethylphenyl)-1-phenylethyl)piperazine\|MK2a Inhibitor\|H-Glu(1)-Ser-Ile-Tyr-Asp-Pro-Gly-Asp- | |
| | Asp-Ile-Lys(1)-OH\|N-(4-ethoxybutyl)-2-oxohexadecanamide\|7-(3,5-Dimethoxyphenyl)-2-methyl- | |
| | 1,6-naphthyridine\|4-lodo-2,6-bis(isopropyl)phenol\|Isosorbide-di-(butylcarbamate)\|Ethacrynic | |
| | acid\|Cizolirtine\|Belzutifan\|11-methyl-6,11-dihydro-5H-benzo[a]carbazol-9-ol\|2-Methoxy-5-(3,4,5- | |
| | trimethoxybenzyl)phenol\|5-methyl-3,4-dihydroisoquinolin-1(2H)-one\|L-Cysteinamide,L-arginyl-L- | |
| | cysteinyl-3-cyclohexyl-L-alanylglycylglycyl-L-arginyl-L-isoleucyl-L-alpha-aspartyl-L-arginyl-L- | |
| | isoleucyl-L-phenylalanyl-L-arginyl-,cyclic(2-13)-disulfide\|4-lodo-acetamido phenylboronic acid\|2- | |
| | (2-(2-Fluorophenoxy)phenyl)acetic acid\|2-morpholino-4H-benzo[g]chromen-4- | |
| | one\|Ceftazidime\|Boc-D-Glu-Tyr(SO3H)-Nle-D-Lys-Trp-Nle-Asp-Phe-NH2\|N-[(1S,2S)-2-[(6- | |
| | Fluoro-1,3-benzothiazol-2-yl)amino]cyclopentyl]-2,6-dimethoxy-N- | |
| | methylbenzamide\|Finerenone\|Tert-butyl 2-(2-oxohexadecanoylamino)acetate\|Chromeno[2,3- | |
| | d]pyrimidine-2,4-dione\|Glycopeptide\|N-(3,4-dimethoxybenzyl)-N- | |
| | isopentylamine\|Cianidanol\|Idasanutlin\|6-Heptyne-2,5-diamine\|Org-24598(lithium)\|1-Benzyl-4- | |
| | chroman-2-ylmethyl-piperazine\|Fiduxosin hydrochloride\|Loperamide\|Cnicin\|(2S,4R)-N-(1- | |
| | cyanocyclopropyl)-1-[1-(3-fluoro-5-iodopyridin-2-yl)cyclopropanecarbonyl]-4-(2- | |
| | fluorophenyl)sulfonylpyrrolidine-2-carboxamide\|1-(4-Nitrophenyl)-2-phenylethane-1,2-dione\|(2S)- | |
| | N-[(2S,3S)-4-[(2S)-2-(tert-butylcarbamoyl)pyrrolidin-1-yl]-3-hydroxy-4-oxo-1-phenylbutan-2-yl]-2- | |
| | (quinoxaline-2-carbonylamino)butanediamide\|Cefsulodin sodium\|1- | |
| | Propylidenethiosemicarbazide\|1-Butyl-3-methyl-3,7-dihydro-purine-2,6-dione\|Ac- | |
| | [CV(2lgl)QDWGAHRC]T\|Triglyme\|[1-(3-Chloroanilino)-5-methylisoquinolin-4-yl]-piperidin-1- | |
| | ylmethanone\|(+)-N6-(2-(4-(2-Methoxyphenyl)piperazin-1-yl)ethyl)-N6-propyl-4,5,6,7- | |
| | tetrahydrobenzo[d]thiazole-2,6-diamine\|Sulfamic acid 3-(4-hydroxy-benzoyl)-phenyl | |
| | ester\|Pinusolide\|8-(2-Cyclopropyl-ethoxy)-quinolin-2-ylamine\|Etravirine\|U.S. Pat. No. 9,550,796, Example | |
| | 98\|1-(2-Amino-2-cyclohexylacetyl)pyrrolidine-2-carbonitrile\|Acetic Acid\|2′-Nitro-6- | |
| | bromoflavone\|Nitraquazone\|2-methoxyphenyl 10H-phenothiazine-10-carboxylate\|5′-Methoxy-6′- | |
| | (3-(pyrrolidin-1-yl)propoxy)spiro[cyclobutane-1,3′-indol]-2′-amine\|fidancitinib\|1-[5-(2,4-Difluoro- | |
| | phenylsulfanyl)-4-nitro-thiophen-2-yl]-ethanone\|Benzamide,4-(4-(((6-methoxy-2-(2- | |
| | methoxyimidazo(2,1-b)-1,3,4-thiadiazol-6-yl)-4-benzofuranyl)oxy)methyl)-2-thiazolyl)-N,N- | |
| | dimethyl-\|Bozitinib\|propan-2-yl(2S)-2-[(3R,4R,5R)-5-[2-amino-6-(methylamino)purin-9-yl]-4- | |
| | fluoro-3-hydroxy-4-methyloxolan-2-yl]methoxy-phenoxyphosphoryl]amino]propanoate\|17- | |
| | Methoxy-5,7-dioxa-24-azoniahexacyclo[11.11.0.02,10.04,8.014,19.020,24]tetracosa- | |
| | 1(24),3,8,11,13,15,17,19-octaen-18-ol;chloride\|#N/A\|(2S)-2-[[(2S)-2-[(2S)-2-[[(2S,3R)-2-[(2S)-2- | |
| | [[(2S)-2-[(2S)-2-[(2S)-2-[(2S)-2-[(2-acetamidoacetyl)amino]propanoyl]amino]-5-amino-5- | |
| | oxopentanoyl]amino]-3-phenylpropanoyl]amino]-3-hydroxypropanoyl]amino]-6- | |
| | aminohexanoyl]amino]-3-hydroxybutanoyl]amino]propanoyl]amino]propanoyl]amino]-6- | |
| | aminohexanoic acid\|5-[(1S,2R,4R)-7-azabicyclo[2.2.1]heptan-2-yl]-1,2,4-oxadiazol-3-amine\|2-(2- | |
| | ((4-iodophenyl)amino)-2-oxoethoxy)-N-(2-(methyl(phenyl)amino)-2- | |
| | oxoethyl)benzamide\|Selgantolimod\|(2~{R})-2-[5-[3-chloranyl-2-methyl-4-[2-(4-methylpiperazin-1- | |
| | yl)ethoxy phenyl]-6-(4-fluorophenyl)thieno[2,3-d]pyrimidin-4-yl]oxy-3-[2-[2-(2- | |
| | methoxyphenyl)pyrimidin-4-yl]methoxy]phenyl]propanoic acid\|Hippuristanol\|N,N,N′,N′- | |
| | Tetramethyl-p-phenylenediamine dihydrochloride\|3,5-Dithiocyanatopyridine-2,6-diamine\|9-(4- | |
| | Hydroxybutyl)-N2-phenylguanine\|9H-Xanthene-4-propanoic acid,7-carboxy-3-((6-(4- | |
| | methoxyphenyl)-5-hexenyl)oxy)-9-oxo-,(E)-\|CID 5281560\|2-Tosylanthracene-1,4- | |
| | diol\|trans,trans-Farnesol\|Fipamezole\|1,4,5,6-Tetrahydropyrimidin-2-amine\|5-{[5-(3-Chloro-4- | |
| | methoxyphenyl)furan-2-yl]methylidene}-2-sulfanylidene-1,3-diazinane-4,6-dione\|Stavudine\|1-(3- | |
| | Nitro-benzyl)-1H-indole-5-carboxamidine\|Quinoline,6-(1-methyl-1-(methylsulfonyl)ethyl)-8-(3- | |
| | ((1E)-2-(3-methyl-1,2,4-oxadiazol-5-yl)-2-(4-(methylsulfonyl)phenyl)ethenyl)phenyl)- | |
| | \|Ordopidine\|[(1-Benzyl-5-hydroxy-2-oxo-1,2,3,4-tetrahydro-[1,7]naphthyridine-6-carbonyl)- | |
| | amino]acetic acid\|3-Isopropyl-5-(4-(((6-(4-(methylsulfonyl)phenyl)pyridin-3- | |
| | yl)oxy)methyl)piperidin-1-yl)-1,2,4-oxadiazole\|Lanoconazole\|Uridine,2′-deoxy-5-iodo-[D-Tpi6, | |
| | Leu13 psi(CH2NH)-Leu14]bombesin-(6-14)\|2-Morpholin-4-yl-8-styrylchromen-4-one\|(4-(3,5- | |
| | Bis(trifluoromethyl)phenylsulfonyl)piperazin-1-yl)(phenyl)methanone\|Ombitasvir\|isobutyryl-Arg- | |
| | Phe-Tyr-Arg-Ile-Lys-NH2\|((4-(4-Amidinophenoxy)butanoyl)aspartyl)valine\|D- | |
| | Mannose\|Caffeine\|Ketamine\|2-[2-Carboxy-2-(3-sulfanylpropyl)ethyl]benzoic acid\|Testosterone | |
| | buciclate\|Dansylcadaverine\|(R,S)-1-(5-methyl-1H-indol-1-yl)propan-2-amine\|Autotaxin-IN- | |
| | 1\|Alisporivir\|(12E,20Z,18S)-8-hydroxyvariabilin\|4-(2-aminoethoxy)-3-(4-bromophenyl)-7-fluoro- | |
| | 2H-isoquinolin-1-one;hydrochloride\|SB 269970 hydrochloride\|Crizotinib\|isorhamnetin 3-O- | |
| | rhamnoside\|Elocalcitol\|2-(Furan-2-yl)-7-(3-(4-methoxyphenyl)propyl)-7H-pyrazolo[4,3- | |
| | e][1,2,4]triazolo[1,5-c]pyrimidin-5-amine\|Berberine\|2-Furan-2-yl-3-phenethyl-3H-quinazolin-4- | |
| | one\|Nexturastat A\|methyl(2S)-6-(1-cyclopropylpyrazol-4-yl)-5-(2-fluoroethoxy)-2-methyl-3,4- | |
| | dihydro-2H-quinoline-1-carboxylate\|1-Adamantan-1-yl-3-decyl-urea\|4-benzyl-N-[(4- | |
| | carbamimidoylphenyl)methyl]piperidine-1-carboxamide\|Sodium;5-[(4-amino-3-carboxy-9,10- | |
| | dioxoanthracen-1-yl)amino]-2-anilinobenzenesulfonate\|2-(2-Aminoethyl)thiazole\|Insulin | |
| | glargine\|3-lodo-N(6)-4-aminobenzyladenosine\|3-[5-[(Z)-[5-(anilinocarbamoyl)-2-oxo-1H-indol-3- | |
| | ylidene]methyl]-2,4-dimethyl-1H-pyrrol-3-yl]propanoic acid\|U.S. Pat. No. 10065972, Example 396\|N-(3-((5- | |
| | chloro-2-((4-(4-methylpiperazin-1-yl)phenyl)amino)pyrimidin-4-yl)oxy)phenyl)acrylamide\|alpha- | |
| | (((2-Bromoethyl)amino)methyl)-2-nitro-1H-imidazole-1-ethanol | |
| | monohydrobromide\|Mangostenone G\|1-Benzhydryl-4-(pyridin-2-yl)piperidin-4-ol\|Rigosertib\|2H- | |
| | Pyran-2-one,6-((1E)-4,4-bis(4-fluorophenyl)-3-(1-methyl-1H-tetrazol-5-yl)-1,3-butadien-1- | |
| | yl)tetrahydro-4-hydroxy-,(4R,6S)-rel-\|3-[2-(4-fluorophenyl)ethynyl]-N-[3-(1H-pyrazol-4-yl)propyl]- | |
| | 4-pyridin-4-ylbenzamide\|Cyclopropanecarboxamide,N,N′-((1,1′-biphenyl)-4,4′-diylbis(5,2- | |
| | thiazolediyl-(2S)-2,1-pyrrolidinediyl((1R)-2-oxo-1-phenyl-2,1-ethanediyl)))bis-\|Ziftomenib\|N- | |
| | [(2S)-1-[(2S)-3,3-dimethyl-2-[(2S)-2-(methylamino)propanoyl]amino]butanoyl]pyrrolidin-2- | |
| | yl]methyl]-N-[2-(4-fluorophenyl)ethyl]imidazo[1,2-a]pyrimidine-2-carboxamide\|Glemanserin\|2,6- | |
| | bis(4-tolyl)-9H-purine\|Epetraborole\|(E)-but-2-enedioic acid;[(9R,10S,11S,13S,14S,16R,17R)-9- | |
| | chloro-17-(2-chloroacetyl)-11-hydroxy-10,13,16-trimethyl-3-oxo-6,7,8,11,12,14,15,16- | |
| | octahydrocyclopenta[a]phenanthren-17-yl]furan-2-carboxylate; N-[2-hydroxy-5-[(1S)-1-hydroxy- | |
| | 2-[(2S)-1-(4-methoxyphenyl)propan-2-yl]amino]ethyl]phenyl]formamide; dihydrate\|Ono-AE2- | |
| | 227\|N-(4-methylphenyl)-4-quinolin-4-ylpyrimidin-2-amine\|1-{[4-(1,3-Benzothiazol-2- | |
| | yloxy)phenyl]methyl}piperidin-4-ol\|C200H312N62057 S6\|4-Imidazol-1- | |
| | ylmethylphenylamine\|Calcium acetate\|Analogue A\|2-(1H-pyrazol-3-yl)-1H-benzimidazole\|4- | |
| | Hydroxy-1,2,5-oxadiazole-3-carboxylic acid\|2-[4-[2-(diethylamino)ethoxy]phenyl]-N-[(2,4- | |
| | difluorophenyl)methyl]-5-fluoropyrimidin-4-amine\|Nordihydroguaiaretic acid\|Obeticholic acid\|1,3- | |
| | Bis(nitrooxy)propan-2-yl 2-acetoxybenzoate\|(R)-N-[(1S,2R)-2-Hydroxyindan-1-yl]-3-[(2S,3S)-3- | |
| | (2,6-dimethyl-4-pentylamino-phenoxyacetyl)amino-2-hydroxy-4-phenylbutanoyl]-5,5-dimethyl- | |
| | 1,3-thiazolidine-4-carboxamide\|6H-Thieno(3,2-f)(1,2,4)triazolo(4,3-a)(1,4)diazepine-6- | |
| | acetamide,4-(4-chlorophenyl)-2,3,9-trimethyl-N-(3-(4-methyl-1-piperazinyl)propyl)-,(6S)- | |
| GPA33 | 3,4-Dihydroxy-5-nitrobenzaldehyde\|N-(4-ethylphenyl)-N′-hydroxyoctanediamide\|1-Guanidino-7- | .\|Modulator |
| | phenylsulphamoylisoquinoline\|5-((3,4-Dichlorophenyl)acetyl)-4-(1-pyrrolidinylmethyl)-4,5,6,7- | |
| | tetrahydro-1H-imidazo(4,5-c)pyridine | |
| KIT | Tertatolol\|3-[5-(3-Nitrophenyl)thiophen-2-yl]propanoic acid\|3-Phenyl-1H-benzofuro[3,2- | Inhibitor\| |
| | c]pyrazole\|4-cyano-N-(5-(1-(2-(dimethylamino)acetyl)piperidin-4-yl)-2′,3′,4′,5′-tetrahydro-[1,1′- | CAR-T-Cell- |
| | biphenyl]-2-yl)-1H-imidazole-2-carboxamide\|1-(4-((6,7-Dimethoxyquinolin-4-yl)oxy)-2- | Therapy\| |
| | methoxyphenyl)-3-(1-(thiazol-2-yl)ethyl)urea\|N-(3-chlorophenyl)-4-methyl-3-(2-pyrazolo[1,5- | Modulator |
| | a]pyrimidin-6-ylethynyl)benzamide\|3-[(4-chlorophenyl)-(1H-imidazol-2-yl)methylidene]-5-[(1- | |
| | ethylpiperidin-4-yl)amino]-1H-indol-2-one\|1-(2-(5-(2-Methoxyethoxy)benzimidazol-1-yl)quinolin- | |
| | 8-yl)piperidin-4-ylamine\|1-(4-((4-Ethylpiperazin-1-yl)methyl)-3-(trifluoromethyl)phenyl)-3-(4-((6- | |
| | (methylamino)pyrimidin-4-yl)oxy)phenyl)urea\|O-Phospho-L-tyrosine\|5,11-dimethyl-6H-pyrido[4,3- | |
| | b]carbazol-9-ol\|9-Methoxyellipticine\|9-Bromoellipticine\|4-(4-aminophenyl)-1H-indazol-3- | |
| | amine\|Flt-3 Inhibitor II\|6-(2,6-Dichlorophenyl)-2-{[3-(hydroxymethyl)phenyl]amino}-8- | |
| | methylpyrido[2,3-D]pyrimidin-7(8H)-one\|Pyrido(2,3-d)pyrimidin-7(8H)-one,6-(2,6- | |
| | dichlorophenyl)-2-((4-fluorophenyl)amino)-8-methyl-\|Floxuridine\|Motesanib\|2-((2- | |
| | Bromoethyl)(2,4-dinitro-6-((2-(phosphonooxy)ethyl)carbamoyl)phenyl)amino)ethyl | |
| | methanesulfonate\|Osi-930\|Melitracen\|1-Benzyl-3,5-dimethylpiperidin-4-one\|Amuvatinib\|5- | |
| | Methoxytryptoline\|Famitinib\|Ripretinib\|Pexidartinib\|Imatinib\|Semaxanib\|Sorafenib\|Regorafenib\|P | |
| | azopanib hydrochloride\|Ponatinib\|H-DL-Tyr(2,6-diMe)-DL-Tic-DL-Lys(Cbz)-NHPh\|2-chloro-6- | |
| | methyl-N′-[4-methyl-3-[(4-pyridin-3-ylpyrimidin-2-yl)amino]benzoyl]benzohydrazide\|2,4″- | |
| | Dimethoxy-5,3″-dipropyl-biphenyl\|N-[2-(3,4-dihydro-2H-quinolin-1-yl)-2-oxoethyl]-2-[3-[(Z)- | |
| | hydroxyiminomethyl]phenyl]carbamoylamino]-N-quinolin-8-ylacetamide\|Tinidazole\|2-(3- | |
| | Chlorophenyl)histamine\|2-iodo-6-methyl-N′-[4-methyl-3-[(4-pyridin-3-ylpyrimidin-2- | |
| | yl)amino]benzoyl]benzohydrazide | |
| FGFR2 | 1-(2-Chlorophenyl)-N-methyl-N-(1-methylpropyl)-3-isoquinolinecarboxamide\|5-Hydroxyindole-3- | Binder\|.\| |
| | acetic acid\|Actb-1003\|Platelet factor 4 (59-70)\|Unii-3mae0mxi6K\|7-(4-fluoroanilino)-1-[(1R,3R)-3- | Inhibitor\| |
| | hydroxycyclopentyl]-3-(4-methoxyphenyl)-4H-pyrimido[4,5-d]pyrimidin-2-one\|8- | Antagonist\| |
| | Bicyclo[2.2.1]hept-2-yl-2-{4-[4-(3-hydroxypropyl)piperidin-1-yl]phenylamino}-8H-pyrido[2,3- | Modulator\| |
| | d]pyrimidin-7-one\|Lofentanil\|Deuruxolitinib\|Alofanib\|MK-2461\|(R)-6-(4-((4-Ethylpiperazin-1- | Activator |
| | yl)methyl)phenyl)-N-(1-phenylethyl)-7H-pyrrolo[2,3-d]pyrimidin-4-amine\|Debio-1347\|1H- | |
| | Pyrazole-1-ethanol,4-((1E)-2-(5-((1R)-1-(3,5-dichloro-4-pyridinyl)ethoxy)-1H-indazol-3- | |
| | yl)ethenyl)-\|1-(6-Fluoroindol-1-yl)propan-2-amine\|Lucitanib\|Pemigatinib\|3′-Deoxy-3′-(1,2,3- | |
| | triazol-1-yl)thymidine\|3-tert-butyl-9H-carbazole | |
| FCG3A | 3,4-Dibutoxy-3-cyclobutene-1,2-dione\|#N/A\|PF-670462 | Inhibitor\|. |
| PGFRB | L-cis-diltiazem\|1-(4-((6,7-Dimethoxyquinolin-4-yl)oxy)-2-methoxyphenyl)-3-(1-(thiazol-2- | .\|Inhibitor\| |
| | yl)ethyl)urea\|PDGF Receptor Tyrosine Kinase Inhibitor III\|PDGF RTK inhibitor\|Flt-3 Inhibitor II\|3- | Modulator |
| | Phenyl-1H-benzofuro[3,2-c]pyrazole\|1H-Imidazo(4,5-g)quinoxaline,1,2-dimethyl-6-(2-thienyl)-\|3- | |
| | [(4-chlorophenyl)-(1H-imidazol-2-yl)methylidene]-5-[(1-ethylpiperidin-4-yl)amino]-1H-indol-2- | |
| | one\|1-(2-(5-(2-Methoxyethoxy)benzimidazol-1-yl)quinolin-8-yl)piperidin-4-ylamine\|#N/A\|4-[(2S)- | |
| | 2-(3-Chlorophenyl)-2-hydroxyethyl]amino]-3-[7-methyl-5-(4-morpholinyl)-1H-benzimidazol-2-yl]- | |
| | 2(1H)-pyridinone\|(2,4-Dihydroindeno[1,2-c]pyrazol-3-yl)phenylamine\|N-(3-fluorophenyl)-6,7- | |
| | dimethoxy-1,4-dihydroindeno[1,2-c]pyrazol-3-amine\|5-[(Z)-(5-chloro-2-oxo-1,2-dihydro-3H-indol- | |
| | 3-ylidene)methyl]-N-[2-(diethylamino)ethyl]-2,4-dimethyl-1H-pyrrole-3-carboxamide\|4-(3-Bromo- | |
| | phenoxy)-6,7-dimethoxy-quinazoline\|4-(3-Bromo-phenoxy)-6,7-dimethoxy-quinoline\|6,7- | |
| | Dimethoxy-4-(3,4-dimethoxyphenoxy)quinoline\|6,7-dimethoxy-N-methyl-N-phenylquinazolin-4- | |
| | amine;hydrochloride\|6,7-Dimethoxy-3-pyridin-3-yl-quinoline\|4-(6,7-Dimethoxy-quinolin-3-yl)- | |
| | benzoic acid\|6,7-Dimethoxy-3-(4-methoxy-phenyl)-quinoline\|7-Fluoro-3-thiophen-3-yl- | |
| | quinoline\|6,7-Dimethoxy-3-thiophen-2-yl-quinoline\|7-Methoxy-3-thiophen-3-yl-quinoline\|3-(3,4- | |
| | Dimethoxy-phenyl)-6,7-dimethoxy-quinoline\|4-(6,7-Dimethoxyquinolin-3-yl)phenol\|7-Thiophen-3- | |
| | yl-[1,3]dioxolo[4,5-g]quinoline\|6,7-Difluoro-3-thiophen-3-yl-quinoline\|6,7-Dimethoxy-3-p-tolyl- | |
| | quinoline\|6,7-Dimethoxy-3-(3-methoxy-phenyl)-quinoline\|6,7-Dimethoxy-3-phenylethynyl- | |
| | quinoline\|3-Cyclopentyl-6,7-dimethoxy-quinoline\|6-Methoxy-3-thiophen-3-yl-quinoline\|3-(2- | |
| | Cyclohexyl-ethyl)-6,7-dimethoxy-quinoline\|7-Methoxy-3-pyridin-4-yl-quinoline\|6,7-Dimethoxy-3- | |
| | ((E)-styryl)-quinoline\|3-Pyridin-3-yl-quinoline-6,7-diol\|Benzyl-(6,7-dimethoxy-quinolin-3-yl)- | |
| | amine\|5,7-Dimethoxy-3-thiophen-3-yl-quinoline\|3-Cyclopent-1-enyl-6,7-dimethoxy-quinoline\|3-(4- | |
| | Pyridinyl)quinoline\|3-(3-Fluoro-phenyl)-6,7-dimethoxy-quinoline\|5,7-Dimethyl-3-thiophen-3-yl- | |
| | quinoline\|3-Benzyloxy-6,7-dimethoxy-quinoline\|6,7-Dimethoxy-3-phenyl-quinoline\|6,7- | |
| | Dimethoxy-3-thiophen-3-yl-quinoline\|5-(6,7-Dimethoxy-quinolin-3-yl)-1H-pyridin-2-one\|3-(3,4- | |
| | Difluoro-phenyl)-6,7-dimethoxy-quinoline\|3-(4-Fluoro-phenyl)-6,7-dimethoxy-quinoline\|3-(3,4- | |
| | Dichloro-phenyl)-6,7-dimethoxy-quinoline\|3-(3-Thienyl)quinoline\|3-((E)-Styryl)-quinoline\|6- | |
| | Methoxy-3-pyridin-4-yl-quinoline\|7-Chloro-3-pyridin-4-yl-quinoline\|5,7-Dimethoxy-3-(4- | |
| | pyridinyl)quinoline\|3-(1H-indol-3-yl)-6,7-dimethoxy-quinoline\|3-(1H-indol-3-yl)quinoline\|3- | |
| | Cyclohexylethynyl-6,7-dimethoxy-quinoline\|6,7-Dichloro-3-thiophen-3-yl-quinoline\|6,7- | |
| | Dimethoxy-3-(4-nitro-phenyl)-quinoline\|5,6,7-Trimethoxy-3-pyridin-4-yl-quinoline\|3-Pyridin-4-yl- | |
| | quinoline-5,7-diol\|6,7-Dimethoxy-3-pyridin-4-yl-quinoline\|3-Pyridin-4-yl-quinolin-7- | |
| | ol\|Bis(benzo[b]furan-2-yl)methanone\|(benzo[b]furan-2-yl)-(1H-indol-2-yl)methanone\|Bis(5- | |
| | aminobenzo[b]furan-2-yl)methanone\|Bis(5-acetoxybenzo[b]furan-2-yl)methanone\|Bis(6- | |
| | hydroxybenzo[b]furan-2-yl)methanone\|Bis(5-hydroxybenzo[b]furan-2-yl)methanone\|Unii- | |
| | 3mae0mxi6K\|6-(2,6-dichlorophenyl)-8-methyl-2-((3-(methylthio)phenyl)amino)pyrido[2,3- | |
| | d]pyrimidin-7(8H)-one\|(5-fluoro-1H-indol-2-yl)-(1H-indol-2-yl)methanone\|Bis(1H-2- | |
| | indolyl)methanone 12\|(1H-indol-2-yl)(5-phenoxy-1H-indol-2-yl)methanone\|Bis(1H-2- | |
| | indolyl)methanone 11\|(5-hydroxy-1H-indol-2-yl)-(1H-indol-2-yl)methanone\|di(1H-indol-2- | |
| | yl)methanone\|3-(1H-Benzimidazol-2-YL)-1H-indazole\|7-(4-fluoroanilino)-1-[(1R,3R)-3- | |
| | hydroxycyclopentyl]-3-(4-methoxyphenyl)-4H-pyrimido[4,5-d]pyrimidin-2-one\|6-(2,6- | |
| | Dichlorophenyl)-2-{[3-(hydroxymethyl)phenyl]amino}-8-methylpyrido[2,3-D]pyrimidin-7(8H)- | |
| | one\|6-(2,6-Dichlorophenyl)-8-methyl-2-(4-morpholin-4-ylanilino)pyrido[2,3-d]pyrimidin-7- | |
| | one\|Pyrido(2,3-d)pyrimidin-7(8H)-one,6-(2,6-dichlorophenyl)-2-((4-fluorophenyl)amino)-8- | |
| | methyl-\|2-[(4-Ethoxyphenyl)amino]-8-methyl-6-(2,6-dichlorophenyl)pyrido[2,3-d]pyrimidine- | |
| | 7(8H)-one\|2-[4-[2-[Ethyl(acetyl)amino]ethoxy]phenyl]amino]-8-methyl-6-(2,6- | |
| | dichlorophenyl)pyrido[2,3-d]pyrimidine-7(8H)-one\|6-(2,6-Dichlorophenyl)-2-[(4-Fluoro-3- | |
| | Methylphenyl)amino]-8-Methylpyrido[2,3-D]pyrimidin-7(8h)-One\|3-(4-Dimethylamino- | |
| | benzylidenyl)-2-indolinone\|4-(1H-benzimidazol-1-yl)aniline\|1H-Benzimidazole,5-methoxy-1- | |
| | phenyl-\|1-Phenyl-5-hydroxybenzimidazole\|5-Substituted 1-Phenylbenzimidazole 22\|6,7- | |
| | Dimethoxy-4-phenoxy-quinoline\|4-(3-Ethoxy-phenoxy)-6,7-dimethoxy-quinoline\|6,7-Dimethoxy- | |
| | 4-(2-methoxyphenoxy)quinoline\|6,7-Dimethoxy-4-(3-nitro-phenoxy)-quinoline\|6,7-Dimethoxy-4- | |
| | m-tolyloxy-quinoline\|3-(6,7-Dimethoxy-quinolin-4-yloxy)-phenylamine\|4-(3-Fluoro-phenoxy)-6,7- | |
| | dimethoxy-quinoline\|6,7-Dimethoxy-4-(4-methoxyphenoxy)quinoline\|4-(3-Ethyl-phenoxy)-6,7- | |
| | dimethoxy-quinoline\|4-(2,3-Dimethoxy-phenoxy)-6,7-dimethoxy-quinoline\|4-(3,5-Dimethoxy- | |
| | phenoxy)-6,7-dimethoxy-quinoline\|3-(6,7-Dimethoxy-quinolin-4-yloxy)-phenol\|6,7-Dimethoxy-4- | |
| | (3-methoxyphenoxy)quinoline\|Bis(5-methoxybenzo[b]furan-2-yl)methanone\|1-Phenyl-1H- | |
| | benzoimidazole\|3-benzyl-N-(2-morpholinoethyl)quinoxalin-2-amine\|[2- | |
| | (Octadecoxymethyl)oxolan-2-yl]methyl 2-(trimethylazaniumyl)ethyl phosphate\|1-[1-[4-(1- | |
| | acetylpiperidin-4-yl)oxy-2-methoxybenzoyl]piperidin-4-yl]-4H-3,1-benzoxazin-2-one\|Tak-593\|MK- | |
| | 2461\|Indiplon\|5-Methoxytryptoline\|Famitinib\|Lucitanib\|Semaxanib\|Rolipram\|Sorafenib\|(Z)-3-(3,4- | |
| | Dimethoxy-phenyl)-2-pyridin-3-yl-acrylonitrile\|6,7-Dimethyl-2-phenylquinoxaline\|Sunitinib\|H-DL- | |
| | Tyr(2,6-diMe)-DL-Tic-DL-Lys(Cbz)-NHPh\|2-chloro-6-methyl-N′-[4-methyl-3-[(4-pyridin-3- | |
| | ylpyrimidin-2-yl)amino]benzoyl]benzohydrazide\|2-iodo-6-methyl-N′-[4-methyl-3-[(4-pyridin-3- | |
| | ylpyrimidin-2-yl)amino]benzoyl]benzohydrazide | |
| PDCD1 | (2S)-2-[(2S)-2-[[(2S)-2-[(2S,3R)-2-[[(2S)-2-[[(2S)-2-[(2S)-2-[(2S)-2-[(2S)-2-[(2- | Inhibitor\|.\| |
| | acetamidoacetyl)amino]propanoyl]amino]-5-amino-5-oxopentanoyl]amino]-3- | Modulator\| |
| | phenylpropanoyl]amino]-3-hydroxypropanoyl]amino]-6-aminohexanoyl]amino]-3- | Antagonist |
| | hydroxybutanoyl]amino]propanoyl]amino]propanoyl]amino]-6-aminohexanoic | |
| | acid\|Levodopa\|Leniolisib\|#N/A\|8-Dibenzofuran-4-yl-2-morpholin-4-ylchromen-4-one\|Benzamide, | |
| | N-((1R)-1-(6-chloro-1H-benzimidazol-2-yl)-2-methoxyethyl)-3-methyl-4-(3-oxo-4-morpholinyl)-, | |
| | sodium salt (1:1)\|(E)-but-2-enedioic acid;[4-[(1S)-1-(methylamino)-3-(4- | |
| | nitrophenoxy)propyl]phenyl]N,N-dimethylcarbamate\|(E)-3-(4- | |
| | (methylsulfonyl)styryl)thiophene\|(3aR,6S,7aS)-N-tert-butyl-5-[(2R,3R)-2-hydroxy-3-[(3-hydroxy- | |
| | 2-methyl-benzoyl)amino]-4-phenylsulfanyl-butyl]-3,3a,4,6,7,7a-hexahydro-2H-thieno[3,2- | |
| | c]pyridine-6-carboxamide; methanesulfonic acid\|(4S)-4-(cyclopropylsulfonylamino)-N-(2- | |
| | methoxyethyl)-4-[5-(2-methoxyphenyl)-1,3,4-oxadiazol-2-yl]butanamide\|(2S,4S)-N-[(1S)-3- | |
| | amino-3-oxo-1-(3-pyrrolidin-1-yl-1,2,4-oxadiazol-5-yl)propyl]-4-hydroxypyrrolidine-2- | |
| | carboxamide\|(4S)-4-(cyclopropylsulfonylamino)-4-[5-(2-methoxyphenyl)-1,3,4-oxadiazol-2-yl]-N- | |
| | (pyridin-3-ylmethyl)butanamide\|6-Acetyl-7-ethoxy-2H-chromen-2-one\|Reversine\|7-Fluoro-2-(4- | |
| | methoxyphenyl)chroman-4-one\|Salmeterol\|Deoxy Risedronic Acid\|4-(3-phenyl-1H-indol-2- | |
| | yl)phenol\|3-cyano-5-fluoro-N-m-tolylbenzamide\|[125I]N,N-Diethyllysergamide\|Antanal 2\|4-octyl- | |
| | N-quinolin-3-ylbenzamide\|Benzal chloride\|Piperlongumine\|(6R,7R)-7-[[(2E)-2-(2-amino-5-chloro- | |
| | 1,3-thiazol-4-yl)-2-[3-[(1S,2R,18R,19R,22S,25R,28R,40S)-48-[(2S,3R,4S,5S,6R)-3- | |
| | [(2S,4S,5S,6S)-4-amino-5-hydroxy-4,6-dimethyloxan-2-yl]oxy-4,5-dihydroxy-6- | |
| | (hydroxymethyl)oxan-2-yl]oxy-22-(2-amino-2-oxoethyl)-5,15-dichloro-2,18,32,35,37- | |
| | pentahydroxy-19-[(2R)-4-methyl-2-(methylamino)pentanoyl]amino]-20,23,26,42,44-pentaoxo- | |
| | 7,13-dioxa-21,24,27,41,43- | |
| | pentazaoctacyclo[26.14.2.23,6.214,17.18,12.129,33.010,25.034,39]pentaconta- | |
| | 3,5,8(48),9,11,14,16,29(45),30,32,34(39),35,37,46,49-pentadecaene-40- | |
| | carbonyl]amino]propoxyimino]acetyl]amino]-8-oxo-3-(pyridin-1-ium-1-ylmethyl)-5-thia-1- | |
| | azabicyclo[4.2.0]oct-2-ene-2-carboxylate\|Penoxsulam\|3,5-Dithiocyanatopyridine-2,6- | |
| | diamine\|Evenamide\|\|bezapolstat\|Dichloroacetate\|Gallium citrate\|Zotatifin\|Alofanib\|2-[(4- | |
| | Methylphenyl)sulfonylamino]-n-(4-phenyl-1,3-thiazol-2-yl)-4-(trifluoromethyl)benzamide\|(S)-3-(4- | |
| | ((5-((8-Methoxy-3,4-dihydroquinolin-1(2H)-yl)methyl)thiophen-2-yl)methoxy)phenyl)hex-4-ynoic | |
| | acid\|(S,E)-N-(1-((5-(2-((4-Cyanophenyl)amino)-4-(propylamino)pyrimidin-5-yl)pent-4-yn-1- | |
| | yl)amino)-1-oxopropan-2-yl)-4-(dimethylamino)-N-methylbut-2-enamide\|Orexin-B\|Rusfertide\|2,7- | |
| | Bis(butyramido)anthraquinone\|4-(Butylthio)-2-methoxybenzonitrile\|Vincamine\|PF-670462\|LY | |
| | 2880070[Who-DD]\|Alstiphyllanine E\|Jnk-IN-1\|Tirofiban\|4-(2-(Benzyloxy)-6- | |
| | fluorophenyl)piperidine\|8-bromo-9-isobutyl-9H-purin-6-amine\|N-(4-iodophenyl)-5-nitrofuran-2- | |
| | carboxamide\|4-[5-(1-Methylpyrazol-4-yl)-3-[2-(4-methyltriazol-1-yl)ethyl]imidazol-4- | |
| | yl]benzonitrile\|Clocortolone\|4-(1H-indol-3-yl)-N,N-dimethylcyclohex-3-enamine\|Cefbuperazone | |
| | sodium\|2-[2-[2-butyl-4-[(E)-3-hydroxy-2-[(6-methoxy-1,3-benzodioxol-5-yl)methyl]-3-oxoprop-1- | |
| | enyl]pyrazol-3-yl]-5-methoxyphenoxy]methyl]benzoic acid\|Indolo[3,2-b]carbazole-6- | |
| | carbaldehyde\|Aurigene NP-12\|(1R,3R)-3-(3,4-dichlorobenzoyl)-2,2-dimethylcyclopropane-1- | |
| | carboxylic acid\|Sufentanil\|Gabazine\|(2-Phenylphenyl)-(1,3,4,9-tetrahydropyrido[3,4-b]indol-2- | |
| | yl)methanone\|Ac-I[CV(1Nal)QDWGAHRC]T\|bromo-deaza-SAH\|c[Nle-Asp-D-Phe-Arg-Trp-Glu]- | |
| | NH2\|2,6-diamino-5-nitropyrimidin-4(3H)-one\|Plecanatide\|[(1S,2R,3S,4S,6R,7R,8R,14R)-4- | |
| | ethenyl-3-hydroxy-2,4,7,14-tetramethyl-9-oxo-6-tricyclo[5.4.3.01,8]tetradecanyl]2-[1-[(2R)-2- | |
| | amino-3-methylbutanoyl]piperidin-3-yl]sulfanylacetate | |
| CD1A | 4-Hydroxy-3-(4-hydroxy-3-nitrocinnamoyl)-6-methyl-2H-pyran-2-one | Binder |
| GPVI | Benzofurazan,4-(4-methyl-1-piperazinyl)-7-nitro-,3-oxide\|cacospongionolide E | Inhibitor |
| CNTFR | Sch-37370\|7-(3,5-Dihydroxyphenyl)naphthalene-1,3-diol | Agonist\| |
| | | Modulator |
| VGFR1 | 5-[(Z)-(5-chloro-2-oxo-1,2-dihydro-3H-indol-3-ylidene)methyl]-N-[2-(diethylamino)ethyl]-2,4- | Inhibitor\| |
| | dimethyl-1H-pyrrole-3-carboxamide\|2-(Pyridin-4-Ylmethylamino)-~{n}-[3- | Activator\| |
| | (Trifluoromethyl)phenyl]benzamide\|Piribedil\|N-[4-[5-(4-bromophenyl)-1-(4- | Modulator\|. |
| | sulfamoylphenyl)pyrazol-3-yl]methyl]phenyl]benzamide\|1-[4-Amino-2,6-di(propan-2-yl)phenyl]-3- | |
| | [1-butyl-4-[3-(4-methylpentoxy)phenyl]-2-oxo-1,8-naphthyridin-3-yl]urea\|Cep- | |
| | 11981\|Zanoterone\|Foliglurax\|CID | |
| | 6509858\|Fascaplysin\|Famitinib\|Sulfatinib\|Lucitanib\|Fruquintinib\|Tivozanib\|3-[(4-chlorophenyl)- | |
| | (1H-imidazol-2-yl)methylidene]-5-[(1-ethylpiperidin-4-yl)amino]-1H-indol-2-one\|7-Chloro-3-oxo-8- | |
| | (1,3-thiazol-5-ylmethylamino)-11,16-dioxa-2,4,19,21-tetrazatricyclo[15.3.1.05,10]henicosa- | |
| | 1(20),5,7,9,17(21),18-hexaene-18-carbonitrile\|Cdk1/2 Inhibitor III\|Pyrazolo[1,5-a]pyrimidine | |
| | 1a\|N-[2,4-dichlorobenzoyl]phenylsulfonamide\|4-Chloro-N-(2- | |
| | chlorobenzoyl)benzenesulfonamide\|3-chloro-N-(4-chlorophenyl)sulfonylbenzamide\|4-Chloro-N- | |
| | [(4-chlorophenyl)sulfonyl]benzamide\|N-(4-chlorobenzenesulfonyl)-2-methylbenzamide\|N,5- | |
| | diphenyl-1,3-oxazol-2-amine\|2-[(5-Phenyl-1,3-oxazol-2-yl)amino]benzonitrile\|N-(2- | |
| | methoxyphenyl)-5-phenyl-1,3-oxazol-2-amine\|3-[(5-Phenyl-1,3-oxazol-2-yl)amino]benzonitrile\|3- | |
| | (5-Phenyl-oxazol-2-ylamino)phenylmethanol\|N-(3-methylphenyl)-5-phenyl-1,3-oxazol-2-amine\|3- | |
| | bromo-N-(4-chlorophenyl)sulfonylbenzamide\|N-(4-chlorophenyl)sulfonyl-4-nitrobenzamide\|4-[(5- | |
| | Phenyl-1,3-oxazol-2-yl)amino]benzenesulfonamide\|Lycogarubin B\|N-(4-chloro-2-fluorophenyl)- | |
| | 6,7-dimethoxyquinazolin-4-amine\|4-(4-(4-Tert-butylphenylamino)phthalazin-1- | |
| | yl)benzamide\|c[homoPhe-Hca-Glu-Gly-Leu-Glu-Glu]-NH2\|N3-GHQMFYYPra-NH2\|(4- | |
| | Chlorophenyl)-(4-isoquinolin-5-yl-phthalazin-1-yl)amine\|4-(isoquinolin-5-yl)-N-m-tolylphthalazin- | |
| | 1-amine\|4-(isoquinolin-5-yl)-N-o-tolylphthalazin-1-amine\|3-(1H-Benzimidazol-2-YL)-1H- | |
| | indazole\|N(1)Tyr-Tyr-Asp-Glu-Lys-Leu-Glu-Glu(1)-NH2\|c[YYAEGLEE]-NH2\|c[YYDEGLEE]- | |
| | NH2\|MK-2461\|2-Pyridin-2-yl-6H-pyrazolo[1,5-c]quinazolin-5-one\|Vatalanib\|Semaxanib | |
| NRG1 | Clobetasol propionate\|[33P]2-methylthio-ADP\|6-chloro-2-methoxy-N-(3-pyridyl)acridin-9-amine | Modulator |
| CD7 | 4-(4-Fluorophenyl)-1-(4-piperidinyl)-5-(2-amino-4-pyrimidinyl)-imidazole\|Cnicin | CAR-T-Cell- |
| | | Therapy |
| BTLA | (O10eq)-beta-guanidinopropionylryanodine | Inhibitor |
| VCAM1 | Agtad[cfwkyc]V\|[4-[3,4-Difluoro-2-(trifluoromethyl)phenyl]piperidin-1-yl]-[5-(3,3,3-trifluoropropyl)- | .\|Inhibitor |
| | 1,4,6,7-tetrahydropyrazolo[4,3-c]pyridin-3-yl]methanone\|N-{2-(4-Methoxy-phenyl)-1-[(pyridin-3- | |
| | ylmethyl)-carbamoyl]-vinyl}-benzamide\|8-(3-Chlorobenzyloxy)caffeine\|3-((2- | |
| | Phenethylphenoxy)methyl)pyridine\|4-Phenyl-1-(1-phenylpentyl)piperidin-4- | |
| | ol\|Benzenebutanamine,N-ethyl-N-heptyl-4-nitro-\|Cholecystokinin-9\|Symbiopolyol | |
| PTK7 | 2-(3-Fluoro-4-hydroxy-phenyl)-benzooxazol-6-ol | Inhibitor |
| KI2L1 | CID 16750271\|Nelonicline\|2-thioUTP | Antagonist\|.\| |
| | | Modulator |
| IL1R1 | L-Arginine,L-phenylalanyl-L-seryl-L-arginyl-L-tyrosyl-L-alanyl-\|4-(4-Fluorophenyl)-2-(4- | Antagonist |
| | methylsulfinylphenyl)-5-(4-pyridyl)-1H-imidazole | |
| PGBM | 4-Amino-2-p-tolylisoindole-1,3-dione | Modulator |
| CD166 | Gallium citrate | . |
| FLT3 | N2-(1H-Indol-5-yl)-6-(pyridin-4-yl)pyrazine-2,3-diamine\|4-cyano-N-(5-(1-(2- | Inhibitor\|.\| |
| | (dimethylamino)acetyl)piperidin-4-yl)-2′,3′,4′,5′-tetrahydro-[1,1′-biphenyl]-2-yl)-1H-imidazole-2- | Modulator\| |
| | carboxamide\|3-(1H-indol-5-yl)-5-(4-((4-methylpiperazin-1-yl)methyl)phenyl)-1H-pyrrolo[2,3- | Binder |
| | b]pyridine\|Flt-3 inhibitor\|Flt-3 Inhibitor II\|(5-Phenyl-thiazol-2-yl)-(4-(2-pyrrolidin-1-yl-ethoxy)- | |
| | phenyl)-amine\|8-Bromo-2-((1-methylpiperidin-4-yl)amino)-4-((4- | |
| | phenoxyphenyl)amino)pyrido[4,3-D]pyrimidin-5(6H)-one\|3-Phenyl-1H-benzofuro[3,2- | |
| | c]pyrazole\|3-[(4-chlorophenyl)-(1H-imidazol-2-yl)methylidene]-5-[(1-ethylpiperidin-4-yl)amino]- | |
| | 1H-indol-2-one\|Benzo[c]-2,6-naphthyridin-5-amine,N-(2-chlorophenyl)-7-(1H-1,2,4-triazol-5-yl)- | |
| | \|1-(4-((4-Ethylpiperazin-1-yl)methyl)-3-(trifluoromethyl)phenyl)-3-(4-((6-(methylamino)pyrimidin- | |
| | 4-yl)oxy)phenyl)urea\|N-[(1S)-1-(5-fluoropyridin-2-yl)ethyl]-3-(3-propan-2-yloxy-1H-pyrazol-5- | |
| | yl)imidazo[4,5-b]pyridin-5-amine\|Bis(benzo[b]furan-2-yl)methanone\|(benzo[b]furan-2-yl)-(1H- | |
| | indol-2-yl)methanone\|Bis(5-aminobenzo[b]furan-2-yl)methanone\|Bis(5-acetoxybenzo[b]furan-2- | |
| | yl)methanone\|Bis(6-hydroxybenzo[b]furan-2-yl)methanone\|Bis(5-hydroxybenzo[b]furan-2- | |
| | yl)methanone\|4-(4-aminophenyl)-1H-indazol-3-amine\|N-[4-(3-amino-1H-indazol-4- | |
| | yl)phenyl]benzamide\|(5-fluoro-1H-indol-2-yl)-(1H-indol-2-yl)methanone\|Bis(1H-2- | |
| | indolyl)methanone 12\|(1H-indol-2-yl)(5-phenoxy-1H-indol-2-yl)methanone\|Bis(1H-2- | |
| | indolyl)methanone 11\|(5-hydroxy-1H-indol-2-yl)-(1H-indol-2-yl)methanone\|di(1H-indol-2- | |
| | yl)methanone\|2-(3,4-Dimethoxybenzamido)thiophene-3-carboxamide\|(Z)-5-Chloro-3-((3,5- | |
| | dimethyl-1H-pyrrol-2-yl)methylene)indolin-2-one\|L-Tryptophanamide,N-acetyl-D-tyrosyl-(4R)-4- | |
| | hydroxy-L-prolyl-L-asparaginyl-L-threonyl-L-phenylalanyl-2-azaglycyl-L-leucyl-N5- | |
| | (imino(methylamino)methyl)-L-ornithyl-\|Tandutinib\|5-oxo-1-tosyl-2,5-dihydro-1Hpyrrol-2-yl | |
| | acetate\|4-Pyridin-2-yl-5-quinolin-2-yl-thiazol-2-ylamine\|Urea,N-(2-methoxy-5-methylphenyl)-N′- | |
| | (6-((6-methoxy-7-(3-(4-methyl-1-piperazinyl)propoxy)-4-quinazolinyl)amino)-2-benzothiazolyl)- | |
| | \|(E)-(4-(2-(1H-indazol-3-yl)vinyl)phenyl)(piperazin-1-yl)methanone\|3-(1H-indol-5-yl)-5-(3,4,5- | |
| | trimethoxyphenyl)-1H-pyrrolo[2,3-b]pyridine\|1-[(Trans-4-Aminocyclohexyl)methyl]-N-Butyl-3-(4- | |
| | Fluorophenyl)-1h-Pyrazolo[3,4-D]pyrimidin-6-Amine\|Unii-uch3qqh1C0\|N-[4-[(4-methylpiperazin- | |
| | 1-yl)methyl]phenyl]-4-(7H-pyrrolo[2,3-d]pyrimidin-4-ylamino)-1H-pyrazole-5- | |
| | carboxamide\|2MT30Ehi63\|MK-2461\|(E)-5-(3-Fluorostyryl)isatin\|(S,E)-N-(1-((5-(2-((4- | |
| | Cyanophenyl)amino)-4-(propylamino)pyrimidin-5-yl)pent-4-yn-1-yl)amino)-1-oxopropan-2-yl)-4- | |
| | (dimethylamino)-N-methylbut-2-enamide\|1(2H)-Isoquinolinone,5,8-dichloro-2-((1,2-dihydro-4- | |
| | methoxy-6-methyl-2-oxo-3-pyridinyl)methyl)-3,4-dihydro-7-((R)-methoxy-3-oxetanylmethyl)-\|2-(1- | |
| | (Aminomethyl)-3-butylcyclopentyl)acetic acid\|(3R,4R)-4-Amino-1-[4-[(3- | |
| | methoxyphenyl)amino]pyrrolo[2,1-f][1,2,4]triazin-5-yl]methyl]piperidin-3-ol\|Tiludronic | |
| | Acid\|Famitinib\|Mivavotinib\|Pacritinib\|Quizartinib\|Pexidartinib\|Midostaurin\|Zidovudine\|Nintedanib\| | |
| | Lestaurtinib\|Ponatinib\|Gilteritinib\|6,7-Dimethyl-2-phenylquinoxaline\|H-DL-Tyr(2,6-diMe)-DL-Tic- | |
| | DL-Lys(Cbz)-NHPh\|Axitinib | |
| HAVR2 | (2S)-2-[(2S)-2-[(2S)-2-[(2S,3R)-2-[(2S)-2-[[(2S)-2-[(2S)-2-[(2S)-2-[(2S)-2-[(2- | Inhibitor\|. |
| | acetamidoacetyl)amino]propanoyl]amino]-5-amino-5-oxopentanoyl]amino]-3- | |
| | phenylpropanoyl]amino]-3-hydroxypropanoyl]amino]-6-aminohexanoyl]amino]-3- | |
| | hydroxybutanoyl]amino]propanoyl]amino]propanoyl]amino]-6-aminohexanoic acid\|benzaldehyde | |
| | 0-4-(heptyloxy)phenylcarbamoyl oxime\|Boc-N-methyl-L-valine\|3,5-Dithiocyanatopyridine-2,6- | |
| | diamine\|Magnesium citrate\|Tyk2-IN-8\|Dimethylcurcumin\|Glucagon (1-37) (porcine) | |
| L1CAM | Adipate\|3-(3,5-Dimethylphenyl)-1-propylpiperidine | CAR-T-Cell- |
| | | Therapy |
| OX2G | CID 11185529 | . |
| CEAM1 | Dichloroacetate | Inhibitor |
| UFO | Furnidipine\|N-[4-(6,7-dimethoxyquinolin-4-yl)oxy-3-fluorophenyl]-4-ethoxy-1-(4-fluoro-2- | .\|Inhibitor\| |
| | methylphenyl)pyrazole-3-carboxamide\|Ceftobiprole medocaril free acid\|Belvarafenib\|N,N,N′,N′- | Modulator |
| | Tetramethyl-p-phenylenediamine dihydrochloride\|8-(3-Hydroxyphenyl)-2-morpholin-4- | |
| | ylchromen-4-one\|cyclo[Cys6,Cys10]N/OFQ(1-13)NH2\|7-fluoro-2-(4-fluorophenyl)-4H-chromene- | |
| | 4-thione\|N-[4-(6,7-dimethoxyquinolin-4-yl)oxy-3-fluorophenyl]-3-(4-fluorophenyl)-2,4-dioxo-1- | |
| | propan-2-ylpyrimidine-5-carboxamide\|(1s,2s)-2-(3,4-Dichlorobenzoyl)cyclopropanecarboxylic | |
| | Acid\|(2S)-2-[(2S)-2-[(2S)-2-[(2S,3R)-2-[(2S)-2-[(2S)-2-[(2S)-2-[(2S)-2-[(2S)-2-[(2- | |
| | acetamidoacetyl)amino]propanoyl]amino]-5-amino-5-oxopentanoyl]amino]-3- | |
| | phenylpropanoyl]amino]-3-hydroxypropanoyl]amino]-6-aminohexanoyl]amino]-3- | |
| | hydroxybutanoyl]amino]propanoyl]amino]propanoyl]amino]-6-aminohexanoic acid\|N-(3-(9H- | |
| | carbazol-9-yl)-2-hydroxypropyl)-N-(furan-2-ylmethyl)methanesulfonamide\|2-((5-chloro-2-((4-((4- | |
| | methylpiperazin-1-yl)methyl)phenyl)amino)pyrimidin-4-yl)amino)-N,N- | |
| | dimethylbenzenesulfonamide\|N~1~-({5-[4,4-bis(ethoxymethyl)cyclohexyl]-1H-pyrazol-4- | |
| | yl}methyl)-N~1~,N~2~-dimethylethane-1,2-diamine\|N-(5-((6,7-Dimethoxyquinolin-4- | |
| | yl)oxy)pyridin-2-yl)-2,5-dioxo-1-phenyl-1,2,5,6,7,8-hexahydroquinoline-3- | |
| | carboxamide\|Bemcentinib\|N-(3-fluoro-4-(2-(1-methyl-1H-imidazol-4-yl)thieno[3,2-b]pyridin-7- | |
| | yloxy)phenylcarbamothioyl)-2-phenylacetamide\|1-(Benzhydrylamino)ethaniminium | |
| | bromide\|Gilteritinib | |
| NTRK2 | 5-oxo-1-tosyl-2,5-dihydro-1Hpyrrol-2-yl acetate\|(Z)-1-(3-((3-((1H-pyrrol-2-yl)methylene)-2- | Modulator\| |
| | oxoindolin-6-yl)amino)-4-methylphenyl)-3-(2-fluoro-5-(trifluoromethyl)phenyl)urea\|(2R)-2-[5-[6- | Inhibitor |
| | amino-5-[(1R)-1-[5-fluoro-2-(triazol-2-yl)phenyl]ethoxy]pyridin-3-yl]-4-methyl-1,3-thiazol-2- | |
| | yl]propane-1,2-diol\|5-chloro-N2-(1-(5-fluoropyridin-2-yl)ethyl)-N4-(5-isopropoxy-1H-pyrazol-3- | |
| | yl)pyrimidine-2,4-diamine\|(3S,4S,5S)-5-(Hydroxymethyl)piperidine-3,4-diol\|5-[4-[2-(4-oxo-2H- | |
| | 1,3-benzoxazin-3-yl)ethoxy]phenyl]methyl]-1,3-thiazolidine-2,4- | |
| | dione\|Aekkdegpyrmehfrwgsppkd\|20-Amino-14-fluoro-4,17-dimethyl-7,10,18-trioxa-4,5,21- | |
| | triazatetracyclo[17.3.1.02,6.011,16]tricosa-1(23),2,5,11(16),12,14,19,21-octaene-3- | |
| | carbonitrile\|3-amino-6-methyl-N-pyridin-2-ylpyrazine-2-carboxamide\|2-[6-(4- | |
| | Chlorophenoxy)hexyl]-1-cyano-3-pyridin-4-ylguanidine\|MK-2461\|Entrectinib\|Larotrectinib\|N-(6- | |
| | fluoro-3-pyridyl)-1-[4-[(5-isopropyl-1H-pyrazol-3-yl)amino]-6,7-dihydro-5H- | |
| | cyclopenta[d]pyrimidin-2-yl]pyrrolidine-2-carboxamide\|4-[3-amino-4-(oxan-4-yloxy)-1H-indazol-6- | |
| | yl]-2-(4-cyclohexylpiperazin-1-yl)-N-methylbenzamide\|4-[3-amino-4-(1,1,1-trifluoropropan-2- | |
| | yloxy)-1H-indazol-6-yl]-2-(4-phenylpiperazin-1-yl)benzamide\|4-[3-amino-4-(oxan-4-yloxy)-1H- | |
| | indazol-6-yl]-2-(4-phenylpiperazin-1-yl)benzamide\|XX8E5WP7FS | |
| SIG15 | Encequidar | Inhibitor |
| MYPC3 | Omecamtiv mecarbil | Modulator |
| CD28 | 1H-Pyrrolo(1,2-a)indole-1-acetic acid, 9-((4-chlorophenyl)thio)-6-fluoro-2,3-dihydro-,(1S)-\|Y-c[D- | CAR-T-Cell- |
| | Pen-(3-1)Tyr-GSFC]KR-NH2\|8-(3,3,3-Trifluoropropoxy)quinolin-2-amine\|8-Benzo[1,3]dioxol-,5- | Therapy(Dual |
| | ylmethyl-9-butyl-9H-\|1-[2-(4-Methylpiperidin-1-yl)-6-(trifluoromethyl)pyridin-3-yl]methyl]-3-[4- | specific)\|.\| |
| | [(sulfamoylamino)methyl]phenyl]urea\|Cilofexor\|3-[8-Amino-1-[4- | Inhibitor\| |
| | [difluoro(phenyl)methyl]phenyl]imidazo[1,5-a]pyrazin-3-yl]-1-methylcyclobutan-1- | Immunomodulator\| |
| | ol\|Zandelisib\|Indiplon\|Benzodepa\|Hydroxy(1-naphthyl)methylphosphonic acid | Modulator\| |
| | | Antagonist |
| NCAM1 | {(3r,4r)-4-Methyl-3-[methyl(7h-Pyrrolo[2,3-D]pyrimidin-4-YI)amino]piperidin-1-YI}(Pyrrolidin-1- | CAR-T-Cell- |
| | YI)methanone\|#N/A\|Phosphoric acid mono-((E)-dec-4-enyl) ester\|13,16,19,22-Tetraoxa-3,6- | Therapy |
| | diazatricyclo[21.3.1.18,12]octacosa-1(27),2,6,8(28),9,11,23,25-octaene-27,28-diol | |
| ROR1 | Lintopride\|4-(3-(4-Hydroxybutyl)-4,4-dimethyl-2,5-dioxoimidazolidin-1-yl)-2- | CAR-T-Cell- |
| | (trifluoromethyl)benzonitrile\|Sibofimloc\|2,3-Diphenyl-1H-indole\|Zeteletinib hemiadipate | Therapy\|.\| |
| | | Antagonist |
| TREM1 | d-Tartaric acid | Inhibitor |
| PD1L1 | 2-(2-((4-iodophenyl)amino)-2-oxoethoxy)-N-(2-(methyl(phenyl)amino)-2- | Inhibitor\| |
| | oxoethyl)benzamide\|U.S. Pat. No. 10,092,575, Example 101\|6-(4-Tert-butylphenoxy)pyridin-3-amine\|1-[3- | CAR-T-Cell- |
| | Bromo-4-(2-methyl-biphenyl-3-ylmethoxy)-benzyl]-piperidine-2-carboxylic acid\|N-{2-[{{2- | Therapy(Dual |
| | Methoxy-6-[(2-Methyl[1,1′-Biphenyl]-3-YI)methoxy]pyridin-3- | specific)\| |
| | Yl}methyl)amino]ethyl}acetamide\|#N/A\|3,5,7-Trihydroxy-2-(4-hydroxyphenyl)-8-(3,4,5-trihydroxy- | Agonist\|.\| |
| | 6-methyloxan-2-yl)oxychromen-4-one\|1-(4-Methoxyphenyl)-3-[(2-oxo-3- | CAR-T-Cell- |
| | indolyl)amino]thiourea\|Cyclizine\|3-Aminopyrrolidine dihydrochloride\|4-[2-Hydroxy-3- | Therapy\| |
| | (isopropylamino)propyloxy]benzoic acid ethyl ester\|1-(Pyridin-4-yl)-3-(quinolin-2-yl)prop-2-en-1- | Modulator |
| | one\|Isoamylamine\|Nerofe\|6H-Thieno(3,2-f)(1,2,4)triazolo(4,3-a)(1,4)diazepine-6-acetamide,4- | |
| | (4-chlorophenyl)-2,3,9-trimethyl-N-(3-(4-methyl-1-piperazinyl)propyl)-,(6S)- | |
| | \|Lisaftoclax\|Octahydrocurcumin\|(2S,3S)-2-Amino-3-methyl-N-(2- | |
| | morpholinoethyl)pentanamide\|Vimseltinib\|Magnesium citrate\|2-[(4-Methylphenyl)sulfonylamino]- | |
| | n-(4-phenyl-1,3-thiazol-2-yl)-4-(trifluoromethyl)benzamide\|N-(5-((6,7-Dimethoxyquinolin-4- | |
| | yl)oxy)pyridin-2-yl)-2,5-dioxo-1-phenyl-1,2,5,6,7,8-hexahydroquinoline-3- | |
| | carboxamide\|Deutarserine\|Methyl Pyridazine-4-carboxylate\|Orelabrutinib\|3-(Furan-3-yl) aniline\|4- | |
| | (5-(4-Methoxyphenyl)thiophen-2-yl)phenol\|4-(Butylthio)-2- | |
| | methoxybenzonitrile\|Clarithromycin\|Penoxsulam\|Abametapir\|8-methyl-4H,7H-indolo[6,5,4- | |
| | cd]indol-5-one\|Bromazine\|Bromosulfophthalein\|(3S)-N-(3,5-difluoro-4-propan-2-yloxyphenyl)-3- | |
| | (hydroxymethyl)-4-(5-methylpyridin-2-yl)-2,3-dihydro-1,4-benzoxazine-8- | |
| | carboxamide\|Ordopidine\|N-arachidonyl-D-alanine\|5-(2-Bromo-4-methylbenzoyl)-2- | |
| | chlorobenzenesulfonamide\|(1R,3S)-3-[2-methoxy-4-(2-methyloctan-2-yl)phenyl]cyclohexan-1- | |
| | ol\|(2S)-1-[(2S)-2-[5-(2,3-dihydro-1-benzofuran-2-yl)-1-ethoxy-1-oxopentan-2- | |
| | yl]amino]propanoyl]pyrrolidine-2-carboxylic acid\|Temuterkib\|3-[4-[3-methyl-1-[4-[4- | |
| | (trifluoromethyl)phenyl]phenoxy]butyl]benzoyl]amino]propanoic Acid\|2-(cyclohexylthiomethyl)-5- | |
| | hydroxy-4H-pyran-4-one\|(1-butyl-5-phenyl-1H-pyrrol-3-yl)(naphthalen-1- | |
| | yl)methanone\|Ethylphenserine\|1-Benzhydryl-4-o-tolylpiperidin-4-ol\|2-(3-Chlorobenzyloxy)-6- | |
| | chloroisonicotinonitrile | |
| FGFR3 | Trapidil\|Cyclopropanolamide,12a\|Actb-1003\|(S)-5-fluoro-2-(1-(5-fluoropyridin-2-yl)ethylamino)- | Inhibitor\|.\| |
| | 6-(5-methyl-1H-pyrazol-3-ylamino)nicotinonitrile\|3-[(3-(2-Carboxyethyl)-4-methylpyrrol-2- | Antagonist |
| | YL)methylene]-2-indolinone\|5,11-dimethyl-6H-pyrido[4,3-b]carbazol-9-ol\|7-(4-fluoroanilino)-1- | |
| | [(1R,3R)-3-hydroxycyclopentyl]-3-(4-methoxyphenyl)-4H-pyrimido[4,5-d]pyrimidin-2-one\|8- | |
| | Bicyclo[2.2.1]hept-2-yl-2-{4-[4-(3-hydroxypropyl)piperidin-1-yl]phenylamino}-8H-pyrido[2,3- | |
| | d]pyrimidin-7-one\|(E)-4-(5-ethoxypyridin-3-yl)-N-methylbut-3-en-1-amine\|MK-2461\|(R)-6-(4-((4- | |
| | Ethylpiperazin-1-yl)methyl)phenyl)-N-(1-phenylethyl)-7H-pyrrolo[2,3-d]pyrimidin-4-amine\|Debio- | |
| | 1347\|Phosphorodiamidic acid,N,N-bis(2-chloroethyl)-N′-(3-hydroxypropyl)-,(3-chloropropyl) | |
| | ester\|Tofisopam\|Dovitinib lactate\|Brivanib alaninate\|Lucitanib\|Pemigatinib | |
| ROR2 | (3R,5S,6E)-3,5-Dihydroxy-7-[1-phenyl-2-isopropyl-4-(4-fluorophenyl)-1H-pyrrol-3-yl]-6-heptenoic | CAR-T-Cell- |
| | acid\|3,4-Dimethoxy-N-[6-(4-Methoxyphenoxy)-1,3-Dimethyl-2-Oxo-2,3-Dihydro-1h- | Therapy |
| | Benzimidazol-5-Yl]benzenesulfonamide | |
| TIE2 | Bis(p-bromophenyl)sulfone\|5-((5-(4-((4-(2-hydroxyethyl)piperazin-1-yl)methyl)-3- | Modulator\| |
| | (trifluoromethyl)phenylcarbamoyl)-2-methylphenyl)ethynyl)-N,1-dimethyl-1H-imidazole-2- | Inhibitor\| |
| | carboxamide\|N-(4-phenoxyphenyl)quinazolin-4-amine\|N-(4-{4-Amino-1-[4-(4-Methylpiperazin-1- | Activator |
| | YI)-Trans-Cyclohexyl]-1h-Pyrazolo[3,4-D]pyrimidin-3-YI}-2-Methoxyphenyl)-1-Methyl-1h-Indole- | |
| | 2-Carboxamide\|2-(4-Methoxyphenyl)-3-phenylmaleimide\|2-(1H-Indole-3-yl)-3- | |
| | phenylmaleimide\|1H-Pyrrole-2,5-dione,3,4-bis(4-methoxyphenyl)-\|3,4-diphenyl-1H-pyrrole-2,5- | |
| | dione\|Mitoquinone\|Pexmetinib\|Altiratinib\|Cep-11981\|Rebastinib\|Razuprotafib | |
| MALT1 | Octahydrocurcumin | Inhibitor |
| LAG3 | 8,9-dichloro-2,3,4,5-tetrahydro-1H-2-benzazepine\|D-Lactic acid\|D-Phe-c[Cys-Tyr-D-Trp-Lys-Val- | Modulator\|.\| |
| | Cys]-Asp-NH2\|Hexachlorophene\|Isoamylamine\|Ceftobiprole medocaril free | Inhibitor\| |
| | acid\|Penoxsulam\|Tyk2-IN-8\|(S)-3-(4-((5-((8-Methoxy-3,4-dihydroquinolin-1(2H)- | Immunomodulator |
| | yl)methyl)thiophen-2-yl)methoxy)phenyl)hex-4-ynoic acid\|N-(3-phenoxy-4- | |
| | pyridinyl)ethanesulfonamide\|Alofanib\|PRX-07034 hydrochloride\|D-Lysinamide,N-(4- | |
| | ((hydrazinylthioxomethyl)amino)benzoyl)-D-tyrosyl-N6-(N-(4-((2-(1H-imidazol-5-yl)ethyl)amino)- | |
| | 1,4-dioxobutyl)glycyl)-D-lysyl-D-alpha-glutamyl-N6-(N-(4-((2-(1H-imidazol-5-yl)ethyl)amino)-1,4- | |
| | dioxobutyl)glycyl)-\|2-Methoxy-4-(p-tolyloxy)benzonitrile | |
| TYRO3 | N-[4-(6,7-dimethoxyquinolin-4-yl)oxy-3-fluorophenyl]-4-ethoxy-1-(4-fluoro-2- | Inhibitor |
| | methylphenyl)pyrazole-3-carboxamide\|N-[4-(6,7-dimethoxyquinolin-4-yl)oxy-3-fluorophenyl]-3-(4- | |
| | fluorophenyl)-2,4-dioxo-1-propan-2-ylpyrimidine-5-carboxamide\|4-[2-(Butylamino)-5-pyridin-2- | |
| | ylpyrimidin-4-yl]amino]butan-1-ol\|3-[2-(Butylamino)-5-pyridin-2-ylpyrimidin-4-yl]amino]propan-1- | |
| | ol\|N-{1-[5-Chloro-8-(3-fluorophenyl)cinnolin-7-yl]ethyl}-9H-purin-6-amine\|2-N-butyl-4-N- | |
| | (piperidin-4-ylmethyl)-5-pyridin-2-ylpyrimidine-2,4-diamine\|4-[(4-{3-cyano-2-[4-(7H-pyrrolo[2,3- | |
| | d]pyrimidin-4-yl)-1H-pyrazol-1-yl]propyl}piperazin-1-yl)carbonyl]-3-fluorobenzonitrile\|4-{3- | |
| | (cyanomethyl)-3-[4-(7H-pyrrolo[2,3-d]pyrimidin-4-yl)-1H-pyrazol-1-yl]azetidin-1-yl}-N-[4-fluoro-2- | |
| | (trifluoromethyl)phenyl]piperidine-1-carboxamide\|{trans-3-(4-{[4-{[(2S)-2-(hydroxymethyl) | |
| | pyrrolidin-1-yl]methyl}-6-(trifluoromethyl) pyridin-2-yl]oxy}piperidin-1-yl)-1-[4-(7H-pyrrolo[2,3- | |
| | d]pyrimidin-4-yl)-1H-pyrazol-1-yl]cyclobutyl}acetonitrile | |
| HLAG | Dichloroacetate | Inhibitor |
| MAG | 5-[2-(dimethylamino)ethoxy]-2-[1-[(4-ethoxy-2,6-difluorophenyl)methyl]-5,6-dihydro-4H- | . |
| | cyclopenta[c]pyrazol-3-yl]-N-pyridin-4-ylpyrimidin-4-amine | |
| IL12B | Senicapoc\|O-methyl N-[[(5S)-3-(4,6-dihydropyrrolo[1,2-a][4,1]benzoxazepin-9-yl)-2-oxo- | Modulator |
| | oxazolidin-5-yl]methyl]carbamothioate\|3-[1-[bis(4-chlorophenyl)methyl]azetidin-3-yl]- | |
| | methylsulfonylamino]-N-(1-hydroxypropan-2-yl)-5- | |
| | (trifluoromethyl)benzamide\|Cridanimod\|Tyrosyl-methionyl(O)-glycyl-ethylphenylalanine-2- | |
| | acetylhydrazide\|Isoproterenol | |
| MYLK | 1-(3-Hydroxybenzyl)-3-(4-(pyridin-4-yl)thiazol-2-yl)urea\|2-[(n- | Inhibitor |
| | Butoxycarbonylmethyl)sulfinyl]thiazolo[5,4-b]pyridine | |
| CD47 | 3-Propargylthymidine\|Boc-N-methyl-L-valine\|Poseltinib\|propan-2-yl(2S)-2-[(3R,4R,5R)-5-[2- | Inhibitor\|.\| |
| | amino-6-(methylamino)purin-9-yl]-4-fluoro-3-hydroxy-4-methyloxolan-2-yl]methoxy- | Blocker |
| | phenoxyphosphoryl]amino]propanoate\|Thymoquinone\|N-(1-((2-(Dimethylamino)ethyl)amino)-2- | |
| | methyl-1-oxopropan-2-yl)-4-(4-(2-methyl-5-((2S,3R,4R,5S,6R)-3,4,5-trihydroxy-6- | |
| | (methylthio)tetrahydro-2H-pyran-2-yl)benzyl)phenyl)-butanamide\|1-[(Trans-4- | |
| | Aminocyclohexyl)methyl]-N-Butyl-3-(4-Fluorophenyl)-1h-Pyrazolo[3,4-D]pyrimidin-6- | |
| | Amine\|Zandelisib\|Penoxsulam\|(1-((2-Methoxy-4- | |
| | (((phenylsulfonyl)amino)carbonyl)phenyl)methyl)-1H-indazol-6-yl)carbamic acid cyclopentyl | |
| | ester\|N-[1-[(2R,4R,5R)-5-(hydroxymethyl)-4-prop-2-ynoxyoxolan-2-yl]-2-oxopyrimidin-4- | |
| | yl]benzamide\|Aclimostat\|Epocarbazolin A | |
| SLAF7 | 4-Acetamido-N-tert-butylbenzamide\|4-(2-Oxo-2-phenylacetyl)benzoic acid\|NSC 632839 | CAR-T-Cell- |
| | hydrochloride\|Methyl Pyridazine-4-carboxylate\|2-[(4-Methylphenyl)sulfonylamino]-n-(4-phenyl- | Therapy\| |
| | 1,3-thiazol-2-yl)-4-(trifluoromethyl)benzamide\|Quisqualic acid\|4-[(4-chlorophenyl)amino]-2-[4- | Inhibitor\|.\| |
| | [(2E)-2-hydroxyiminopropanoyl]piperazin-1-yl]-6-propan-2-yl-5H-pyrrolo[4,3-e]pyrimidin-7-one | Modulator |
| TIE1 | N-[(3R)-8-[5-chloro-4-[2-fluoro-6-(methylcarbamoyl)anilino]pyrimidin-2-yl]amino]-5,5-dimethyl-2- | Inhibitor |
| | oxo-3,4-dihydro-1H-1-benzazepin-3-yl]pyrrolidine-1-carboxamide | |
| VISTA | U.S. Pat No. 9,550,796, Example 98\|3-Aminopyrrolidine dihydrochloride\|4-Formylpyridine | Inhibitor\| |
| | thiosemicarbazone | Antagonist |

TABLE 10C
Exemplary therapeutic agents with associated kinase-related targets or gene products

| | | Mechanism |
|---|---|---|
| Kinase | Drug-name (from CID) | of Action |
| MRCKA | 1-(3-Hydroxybenzyl)-3-(4-(pyridin-4-yl)thiazol-2-yl)urea | Inhibitor |
| ABL1 | N-[4-[(4-ethylpiperazin-1-yl)methyl]-3-(trifluoromethyl)phenyl]-2-(4-pyridin-3- | Inhibitor\|.\| |
| | ylphenoxy)acetamide\|Axitinib\|Myristic acid\|N-(3-fluorophenyl)-6,7-dimethoxy-1,4- | Modulator |
| | dihydroindeno[1,2-c]pyrazol-3-amine\|(E)-3,5,4′-Trimethoxystilbene\|Unii-3mae0mxi6K\|5-[2-(4- | |
| | Hydroxyphenyl)phenyl]benzene-1,3-diol\|FIt-3 Inhibitor II\|4-((5-(3-Chloro-4-methylphenyl)furan-2- | |
| | yl)methylene)-1-(3,4-dimethylphenyl)pyrazolidine-3,5-dione\|4-(3-(Benzyloxy)benzylidene)-1-(3,4- | |
| | dichlorophenyl)pyrazolidine-3,5-dione\|(4Z)-4-[(6-bromo-1,3-benzodioxol-5-yl)methylidene]-1-(4- | |
| | iodophenyl)pyrazolidine-3,5-dione\|4-(4-Iodobenzylidene)-1-(4-iodophenyl)pyrazolidine-3,5- | |
| | dione\|4-Benzylidene-1-(4-iodophenyl)-3,5-pyrazolidinedione\|2-methoxy-4-(methylthio)-N-(5- | |
| | phenethyl-1,3,4-thiadiazol-2-yl)benzamide\|2-iodo-N-(5-phenethyl-1,3,4-thiadiazol-2- | |
| | yl)benzamide\|4-iodo-N-(5-phenethyl-1,3,4-thiadiazol-2-yl)benzamide\|#N/A\|6-(2,6- | |
| | Dichlorophenyl)-2-{[3-(hydroxymethyl)phenyl]amino}-8-methylpyrido[2,3-D]pyrimidin-7(8H)- | |
| | one\|Pyrido(2,3-d)pyrimidin-7(8H)-one,6-(2,6-dichlorophenyl)-2-((4-fluorophenyl)amino)-8- | |
| | methyl-\|Benzamide,3-((1E)-2-(6-(cyclopropylamino)-9H-purin-9-yl)ethenyl)-4-methyl-N-(4- | |
| | (trifluoromethyl)-2-pyridinyl)-\|L-Tryptophanamide,N-acetyl-D-tyrosyl-(4R)-4-hydroxy-L-prolyl-L- | |
| | asparaginyl-L-threonyl-L-phenylalanyl-2-azaglycyl-L-leucyl-N5-(imino(methylamino)methyl)-L- | |
| | ornithyl-\|(+/−)-2-(2-Cinnamamidobenzamido)-3-phenylpropanoic acid\|(E)-(4-(2-(1H-indazol-3- | |
| | yl)vinyl)phenyl)(piperazin-1-yl)methanone\|Rebastinib\|Flumatinib\|Saracatinib\|Adenosine-5′- | |
| | triphosphate\|Bosutinib\|4-[(2,4-Dichloro-5-methoxyphenyl)amino]-6-methoxy-7-{5-[(4- | |
| | methylpiperazin-1-yl)methyl]furan-3-yl}quinoline-3-carbonitrile\|Ponatinib\|N-[3-[4-(3,3- | |
| | difluorocyclobutyl)imidazol-1-yl]-5-(trifluoromethyl)phenyl]-4-methyl-3-[(4-pyridin-3-ylpyrimidin-2- | |
| | yl)amino]benzamide\|Bifeprunox\|[(4R,4aR,7S,7aR,12bS)-9-acetyloxy-3-methyl- | |
| | 1,2,3,4,4a,7,7a,13-octahydro-4,12-methanobenzofuro[3,2-e]isoquinolin-3-ium-7-yl] sulfate\|N-[3- | |
| | fluoro-4-methyl-5-[(4-pyridin-3-ylpyrimidin-2-yl)amino]phenyl]-4-[(4-methylpiperazin-1- | |
| | yl)methyl]benzamide\|4-[(4-ethylpiperazin-1-yl)methyl]-N-[3-fluoro-4-methyl-5-[(4-pyridin-3- | |
| | ylpyrimidin-2-yl)amino]phenyl]-3-(trifluoromethyl)benzamide\|N-[5-[(3,5-difluorophenyl)methyl]- | |
| | 2H-pyrazolo[3,4-b]pyridin-3-yl]-4-(4-methylpiperazin-1-yl)-2-(oxan-4-ylamino)benzamide\|N-(5- | |
| | (3,5-difluorophenylthio)-1-H-pyrazolo[3,4-b]pyridine-3-yl)-4-(4-methylpiperazine-1-yl)-2- | |
| | (tetrahydro-2H-pyran-4-ylamino)benzamide\|methyl 5-[2-methyl-5-[3- | |
| | (trifluoromethyl)benzoyl]amino]phenyl]methylamino]-1H-pyrrolo[2,3-b]pyridine-2-carboxylate\|5- | |
| | {2-methyl-5-[4-(4-methyl-piperazin-1-ylmethyl)-3-trifluoromethyl-benzoylamino]-benzylamino}- | |
| | 1H-pyrrolo[2,3-b]pyridine-2-carboxylic acid\|5-ethyl-2-[(2-methylphenyl)amino]-1,3-thiazol-4(5H)- | |
| | one\|lodamide\|N-hydroxy-8-(2-methoxyphenyl)-8-oxooctanamide\|N-(5-cyclopropyl-1H-pyrazol-3- | |
| | yl)-2-[(4,7-difluoro-2-methyl-1H-indol-5-yl)oxy]-6-(4-methylpiperazin-1-yl)pyrimidin-4-amine\|N-[7- | |
| | (5-hydroxy-2-methylphenyl)isoquinolin-3-yl]cyclopropanecarboxamide\|N-(7-(5-fluoro-4- | |
| | methylpyridin-3-yl)isoquinolin-3-yl)cyclopropanecarboxamide\|(1S,2S)-2-fluoro-N-[7-[4-methyl-6- | |
| | [(1S)-2,2,2-trifluoro-1-hydroxyethyl]pyridin-3-yl]isoquinolin-3-yl]cyclopropane-1-carboxamide\|1- | |
| | cyclopentyl-3-(1H-pyrrolo[2,3-b]pyridin-5-yl)-1H-pyrazolo[3,4-d]pyrimidin-4-amine\|N-(2- | |
| | Hydroxyethyl)-3-(6-((4-(trifluoromethoxy)phenyl)amino)pyrimidin-4-yl)benzamide\|5-((5-(4-((4-(2- | |
| | hydroxyethyl)piperazin-1-yl)methyl)-3-(trifluoromethyl)phenylcarbamoyl)-2- | |
| | methylphenyl)ethynyl)-N,1-dimethyl-1H-imidazole-2-carboxamide\|N-[3-(Trifluoromethyl)-4-[(4- | |
| | methylpiperazino)methyl]phenyl]-3-[(imidazo[1,2-a]pyrazine-5-yl)ethynyl]-4-methylbenzamide\|N- | |
| | [(1S)-1-(5-fluoropyridin-2-yl)ethyl]-3-(3-propan-2-yloxy-1H-pyrazol-5-yl)imidazo[4,5-b]pyridin-5- | |
| | amine\|(8S,9S,12S,13R,14S,16R)-19-amino-13-hydroxy-8,14-dimethoxy-4,10,12,16-tetramethyl- | |
| | 3,20,22-trioxo-2-azabicyclo[16.3.1]docosa-1(21),4,6,10,18-pentaen-9-yl carbamate | |
| MK10 | Forteo\|TCS JNK 5a\|JNK Inhibitor VIII\|Jnk-IN-8\|1,9-Pyrazoloanthrone\|9-(4-Hydroxyphenyl)-2,7- | Inhibitor |
| | phenanthroline\|Phosphoaminophosphonic acid-adenylate ester\|N,3-diphenyl-1H-indazol-6- | |
| | amine\|1,3-Benzothiazol-2-yl(2-((2-(3-pyridinyl)ethyl)amino)-4-pyrimidinyl)acetonitrile\|Acetamide, | |
| | N-(4-amino-5-cyano-6-ethoxy-2-pyridinyl)-\|N-(4-amino-5-cyano-6-phenylpyridin-2- | |
| | yl)acetamide\|6-(2-Fluorophenyl)[1,3]dioxolo[4,5-G]quinolin-8(5h)-One\|5-cyclohexyl-3-(1- | |
| | methylpyrazol-4-yl)-2H-pyrazolo[3,4-b]pyridine\|2-cyclohexyl-7-(1-methylpyrazol-4-yl)-5H- | |
| | pyrrolo[2,3-b]pyrazine\|Cefetamet Pivoxil HCl\|ethyl (7S)-11-oxo-14-(2-trimethylsilylethynyl)- | |
| | 2,4,10-triazatetracyclo[10.4.0.02,6.07,10]hexadeca-1(16),3,5,12,14-pentaene-5- | |
| | carboxylate\|Disodium hydrogen phosphate\|N-[(1S,2R)-2-[(1,3-Benzothiazol-2- | |
| | yl)amino]cyclopentyl]-2,6-dimethoxybenzamide\|5-cyclohexyl-3-(1-methylpyrazol-4-yl)-1H- | |
| | pyrrolo[2,3-b]pyridine\|N,N-dimethyl-4-[3-(1H-pyrazol-4-yl)-1H-pyrrolo[2,3-b]pyridin-5-yl]aniline\|n- | |
| | (4-Chloro-3-(1h-1,2,4-triazol-5-yl)thiophen-2-yl)-2-(isoquinolin-5-yl)acetamide\|n-(4-Bromo-3-(1h- | |
| | 1,2,4-triazol-5-yl)thiophen-2-yl)-2-(2-oxo-6-(trifluoromethyl)quinolin-1(2h)- | |
| | yl)acetamide\|Metamizole\|CID 6509858\|2-(4-chlorophenyl)-6-phenyl-9H-purine\|6-Methyl-4- | |
| | morpholin-4-yl-chromen-2-one\|Magnevist\|Jnk-IN-5\|3-[(E)-4-(dimethylamino)but-2-enoyl]amino]- | |
| | N-[4-[4-(2-phenylpyrazolo[1,5-a]pyridin-3-yl)pyrimidin-2-yl]amino]phenyl]benzamide | |
| M4K1 | Zandelisib\|LY 2880070 [Who-DD] | Inhibitor |
| KPCE | Phorbol-12-myristate\|(18S)-18-(hydroxymethyl)-17-oxa-4,14,21- | Activator\| |
| | triazahexacyclo[19.6.1.17,14.02,6.08,13.022,27]nonacosa-1(28),2(6),7(29),8,10,12,22,24,26- | Inhibitor\| |
| | nonaene-3,5-dione\|Bisindolylmaleimide IV\|2-(4-Methoxyphenyl)-3-phenylmaleimide\|2-(1H- | Stimulator\|. |
| | Indole-3-yl)-3-phenylmaleimide\|1H-Pyrrole-2,5-dione,3,4-bis(4-methoxyphenyl)-\|3,4-diphenyl- | |
| | 1H-pyrrole-2,5-dione\|Phenol,4,4′-(3-methyl-1-butenylidene)bis-\|(E)-3-(4-Hydroxyphenyl)-2,3- | |
| | diphenylacrylonitrile\|4,4′-(Cycloheptylidenemethylene)diphenol\|2-(4-Hydroxyphenyl)-3,3- | |
| | diphenylacrylonitrile\|3,3-Bis(4-methoxyphenyl)-2-phenylpropenenitrile\|2,3,3- | |
| | Triphenylacrylonitrile\|3,3-Bis(4-hydroxyphenyl)-2-phenylprop-2-enenitrile\|Cyclofenil diphenol\|3- | |
| | (1-Methyl-1H-indol-3-yl)-4-[6,7,8,9-tetrahydro-12-methyl-7beta,8alpha- | |
| | (methaniminomethano)pyrido[1,2-a]indol]-10-yl]-1H-pyrrole-2,5-dione\|2,2′:5′,2″-Terthiophene-4- | |
| | carbaldehyde\|2,2′:5′,2″-Terthiophene-4,5″-dicarbaldehyde\|2,2′:5′,2″-Terthiophene-4-methanol\|4- | |
| | Morpholin-4-yl-chromen-2-one\|Bryostatin 1\|Meprobamate\|17-(Hydroxymethyl)-18-oxa-4,14,22- | |
| | triazahexacyclo[20.6.1.17,14.02,6.08,13.023,28]triaconta-1(29),2(6),7(30),8,10,12,23,25,27- | |
| | nonaene-3,5-dione\|(S)-3-(8-(Dimethylaminomethyl)-6,7,8,9-tetrahydropyrido(1,2-a)indol-10-yl)-4- | |
| | (1-methyl-3-indolyl)-1H-pyrrole-2,5-dione hydrochloride\|Chelerythrine\|4-Adamantyl resorcinol\|[2- | |
| | chloro-3-(trifluoromethyl)phenyl]-[(1S,8R)-5-(5-fluoropyrimidin-2-yl)-3,4,6,12- | |
| | tetrazatricyclo[6.3.1.02,6]dodeca-2,4-dien-12-yl]methanone\|Prostratin\|Bisindolylmaleimide i\|8- | |
| | Octyl-benzolactam-V9\|Balanol | |
| ERN1 | 7-formyl-8-hydroxy-5-oxo-1H,2H,3H,4H,5H-chromeno[3,4-c]pyridine-3-carboximidamide\|N~2~- | Inhibitor |
| | 1h-Benzimidazol-5-Yl-N~4~-(3-Cyclopropyl-1h-Pyrazol-5-Yl)pyrimidine-2,4- | |
| | Diamine\|(1S,3aR,6aS)-2-((R)-2-((S)-2-cyclohexyl-2-(pyrazine-2-carboxamido)acetamido)-3,3- | |
| | dimethylbutanoyl)-N-((S)-1-(cyclopropylamino)-1,2-dioxohexan-3- | |
| | yl)octahydrocyclopenta[c]pyrrole-1-carboxamide\|2-Hydroxy-5-(5-pyrimidinyl)benzaldehyde\|2,3- | |
| | Dibromo-6-hydroxy-5-methoxybenzaldehyde\|3-Bromo-8-formyl-7-hydroxy-4-methylcoumarin\|2- | |
| | cyclohexyl-N-(4H-1,2,4-triazol-3-yl)acetamide\|Methyl 3-(5-formyl-6-hydroxynaphthalen-2- | |
| | yl)benzoate\|3-(4-Bromophenyl)-7-hydroxy-2-oxochromene-8-carbaldehyde\|3-(5-formyl-6- | |
| | hydroxynaphthalen-2-yl)-N,N-dimethylbenzamide\|3-(5-formyl-6-hydroxynaphthalen-2-yl)-N- | |
| | propan-2-ylbenzamide\|N-benzyl-4-(5-formyl-6-hydroxynaphthalen-2-yl)-N-methylbenzamide\|2- | |
| | Hydroxy-6-[5-(4-methylpiperazine-1-carbonyl)furan-2-yl]naphthalene-1-carbaldehyde\|N-ethyl-3- | |
| | [8-formyl-7-hydroxy-5-(2-methoxyethoxy)-4-methyl-2-oxochromen-3-yl]propanamide | |
| RON | Squalamine\|Pamidronic acid\|MK-2461 | .\|Inhibitor |
| E2AK1 | 3-{[(2,6-dichlorophenyl)methyl]amino}-6,7-dimethoxy-1H,4H-indeno[1,2-c]pyrazol-4-one | Inhibitor |
| EPHB1 | 1H-Imidazo[2,1-f]purine-2,4(3H,8H)-dione, 7-(5-hydroxy-2-methylphenyl)-8-(2-methoxyphenyl)- | Inhibitor |
| | 1-methyl-\|(2S)-2-[(4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid | |
| FYV1 | 1-deoxy-1-(methylazaniumyl)-D-glucitol [2,2′,2″,2″-(1,4,7,10-tetraazacyclododecane-1,4,7,10- | Inhibitor |
| | tetrayl-kappa(4)N(1),N(4),N(7),N(10))tetraacetato(4-)]gadolinate(1-)\|6-Amino-N-[3-[4-(4- | |
| | morpholinyl)pyrido[3′,2′:4,5]furo[3,2-d]pyrimidin-2-yl]phenyl]-3-pyridinecarboxamide\|Apilimod | |
| | mesylate | |
| SRMS | 2,6-difluoro-3-[(3-fluorophenyl)sulfonylamino]-N-(3-methoxy-1H-pyrazolo[3,4-b]pyridin-5- | Inhibitor |
| | yl)benzamide | |
| KS6A3 | 3-[(4-chlorophenyl)-(1H-imidazol-2-yl)methylidene]-5-[(1-ethylpiperidin-4-yl)amino]-1H-indol-2- | Inhibitor |
| | one\|(5R)-N-[1-[3-(dimethylamino)propyl]benzimidazol-2-yl]-5-methyl-1-oxo-2,3,4,5-tetrahydro- | |
| | [1,4]diazepino[1,2-a]indole-8-carboxamide\|(1S,2S,3R,4R)-3-((5-chloro-2-(((S)-1-methoxy-7- | |
| | morpholino-6,7,8,9-tetrahydro-5H-benzo[7]annulen-2-yl)amino)pyrimidin-4- | |
| | yl)amino)bicyclo[2.2.1]hept-5-ene-2-carboxamide.\|2-((3,5-difluoro-4-hydroxyphenyl)amino)-8- | |
| | isopentyl-5,7-dimethyl-7,8-dihydropteridin-6(5H)-one\|N-(2-methoxypyridin-4-yl)-1-oxo-2,3,4,5- | |
| | tetrahydro-[1,4]diazepino[1,2-a]indole-8-carboxamide\|Hydroxyzine\|Bisphosphonate,9 | |
| KIME | {[(6E)-3,7,11-trimethyldodeca-6,10-dien-1-yl]oxy}phosphonic acid\|3-Hydroxy-5- | Inhibitor |
| | [hydroxy(phosphonooxy)phosphoryl]oxy-3-methylpentanoic acid | |
| STK38 | (3R)-6-methoxy-N-[2-[(1-methylpiperidin-4-yl)methoxy]-4-(1H-pyrazol-4-yl)phenyl]-1,2,3,4- | Inhibitor |
| | tetrahydroisoquinoline-3-carboxamide | |
| ERBB3 | Mebeverine\|H-DL-Pen(1)-Ile-D-Tyr-Asp-D-aThr-Lys-Gly-Lys-D-Asn-Val-D-Leu-D-Cys(1)-OH\|6- | Inhibitor\| |
| | Chloro-2-(3-nitrophenyl)chromen-4-one\|N-Isopropyl-N′-hydroxyguanidine\|Acetic Acid\|cis-2- | Antagonist\|.\| |
| | (Para-fluorophenyl)cyclopropylamine\|(3R)-1-(4-((1-(4-(5-chloropyrimidin-2-yl)-2-methylphenyl)- | Modulator |
| | 4,4,4-trifluorobutyl)amino)benzoyl)piperidine-3-carboxylic acid\|Iodoresiniferatoxin\|3-[(1,3- | |
| | Dibenzyl-5-hydroxy-2-oxo-8-pyridin-3-yl-1,2-dihydro-[1,7]naphthyridine-6-carbonyl)-amino]- | |
| | propionic acid\|4-bromo-1H-indazole\|Tazemetostat\|4,4′-Dimethylbenzil\|2-(2,4-Diethoxyphenyl)- | |
| | 3,5,7-triethoxychromen-4-one\|1-(4-chlorophenyl)-4-(piperidin-1-yl)-1H-imidazol-2(5H)-one\|1- | |
| | deoxy-1-(methylazaniumyl)-D-glucitol[2,2′,2″,2″-(1,4,7,10-tetraazacyclododecane-1,4,7,10- | |
| | tetrayl-kappa(4)N(1),N(4),N(7),N(10)tetraacetato(4-)]gadolinate(1-)\|Simufilam\|4-(4- | |
| | Cyanophenoxy)-3-pyridinesulfonamide\|3-(2-Phenyl-2-(piperazin-1-yl)ethyl)phenol\|4-{[3-(2- | |
| | phenylethynyl)phenyl]methyl}-1H-imidazole\|S-(2-Aminophenyl)-L-cysteine S,S- | |
| | dioxide\|Elisidepsin\|N-(4-Chloro-3-((3-methyl-2-butenyl)oxy)phenyl)-2-methyl-3- | |
| | furancarbothioamide\|Atocalcitol\|N-[4-(4-Phenyl-piperazin-1-yl)-butyl]-benzamide\|4-[1,1-Bis(4- | |
| | hydroxyphenyl)hex-1-en-2-yl]phenol | |
| STK16 | 2-[5-Chloro-2-(1-ethyl-5,5-dimethyl-2-oxo-2,3,4,5-tetrahydro-1H-benzo[b]azepin-8-ylamino)- | Inhibitor |
| | pyrimidin-4-ylamino]-3-fluoro-N-methyl-benzamide | |
| M3K7 | N,3-dimethyl-N-[(2R)-4-(4-methylpiperidin-1-yl)butan-2-yl]benzenesulfonamide\|N-[4-[(4- | Inhibitor |
| | ethylpiperazin-1-yl)methyl]-3-(trifluoromethyl)phenyl]-4-methyl-3-(1H-pyrrolo[2,3-b]pyridin-4- | |
| | yloxy)benzamide\|N-{3-[(5-cyclopropyl-2-{[4-(morpholin-4-yl)phenyl]amino}pyrimidin-4- | |
| | yl)amino]propyl}cyclobutanecarboxamide | |
| TSSK2 | N-[(1S)-1-(5-fluoropyridin-2-yl)ethyl]-3-(3-propan-2-yloxy-1H-pyrazol-5-yl)imidazo[4,5-b]pyridin-5- | Inhibitor |
| | amine | |
| E2AK2 | #N/A\|PKR Inhibitor\|4-(6-Cyclohexylmethoxy-9H-purin-2-ylamino)-N,N-diethylbenzamide\|N-[3-({5- | Inhibitor |
| | [2-(3-hydroxypropyl)-2H-tetrazol-5-yl]pyridin-3-yl}ethynyl)phenyl]-3-methyl-2-furamide\|1-(2-(2- | |
| | amino-5-(2H-tetrazol-5-yl)pyridin-3-yl)benzo[b]thiophen-5-yl)-3-(4-chloro-3- | |
| | (trifluoromethyl)phenyl)urea\|1-[2-Fluoro-5-(trifluoromethyl)phenyl]-3-[4-[2-[5-[2-(3- | |
| | hydroxypropyl)tetrazol-5-yl]pyridin-3-yl]ethynyl]phenyl]urea | |
| PK3CB | 5-(7-(methylsulfonyl)-2-morpholino-6,7-dihydro-5H-pyrrolo[2,3-d]pyrimidin-4-yl)pyrimidin-2- | Inhibitor\| |
| | amine\|2-(4-ethylpiperazin-1-yl)-N-[4-(2-morpholin-4-yl-4-oxochromen-8-yl)dibenzothiophen-1- | Modulator\|. |
| | yl]acetamide\|7-methyl-2-morpholino-9-(1-(phenylamino)ethyl)-4H-pyrido[1,2-a]pyrimidin-4-one\|4- | |
| | (6-((4-(cyclopropylmethyl)piperazin-1-yl)methyl)-2-(5-fluoro-1H-indol-4-yl)thieno[3,2-d]pyrimidin- | |
| | 4-yl)morpholine\|N-[(E)-(6-Bromoimidazo[1,2-a]pyridin-3-yl)methylideneamino]-N,2-dimethyl-5- | |
| | nitrobenzenesulfonamide\|1-cyclopentyl-3-(1H-pyrrolo[2,3-b]pyridin-5-yl)-1H-pyrazolo[3,4- | |
| | d]pyrimidin-4-amine\|(R)-2-(1-(7-methyl-2-morpholino-4-oxo-4H-pyrido[1,2-a]pyrimidin-9- | |
| | yl)ethylamino)benzoic acid\|(R)-8-(1-((3,5-difluorophenyl)amino)ethyl)-N,N-dimethyl-2- | |
| | morpholino-4-oxo-4H-chromene-6-carboxamide\|2-methyl-1-(2-methyl-3-(trifluoromethyl)benzyl)- | |
| | 6-morpholino-1H-benzo[d]imidazole-4-carboxylic acid\|(S)-N-(8-(2-Hydroxy-3- | |
| | morpholinopropoxy)-7-methoxy-2,3-dihydroimidazo[1,2-c]quinazolin-5-yl)-2- | |
| | methylnicotinamide\|Bimiralisib\|Buparlisib\|1-(6-fluoro-3-methylisoquinolin-5-yl)-3-[(1R,3R)-3- | |
| | phenylcyclopentyl]urea\|5-[4-(4-Nitrophenyl)piperazin-1-yl]-5-(4-phenylphenyl)-1,3-diazinane- | |
| | 2,4,6-trione\|Tetraethylene glycol\|Piperoxan\|1-Butyl-8-phenyl-3,7-dihydro-purine-2,6-dione\|6- | |
| | Cyclohexylmethoxy-2-(3′-chloroanilino) purine\|Cyclopentanecarboxamide,4-(4-(((1R)-1-((3- | |
| | chloro-2-thienyl)methyl)propyl)amino)-7H-pyrrolo(2,3-d)pyrimidin-7-yl)-N-ethyl-2,3-dihydroxy-, | |
| | (1S,2R,3S,4R)-\|(RS/SR)-2-[1-(3,4-dichlorophenyl)butyl]piperidine\|4-Chloro-2-nitro-1-((4- | |
| | benzylpiperazinyl)sulfonyl)benzene\|6-[(2R)-2-[2-[4-(4-chlorophenoxy)piperidin-1- | |
| | yl]ethyl]pyrrolidin-1-yl]sulfonyl-1H-indole\|[35S]ATPalphaS\|2-(4-Methoxyphenyl)-1-(1-pentylindol- | |
| | 3-YL)ethanone\|N-[1-(2-ethoxyethyl)-4,6-dimethyl-5-(sulfamoylamino)-2,3-dihydroindol-7-yl]-2,2- | |
| | dimethylpropanamide\|2-Imino-8-methyl-10-oxo-1-phenethyl-1,10-dihydro-2H-1,9,10a-triaza- | |
| | anthracene-3-carboxylic acid (furan-2-ylmethyl)-amide\|Garcinone D\|5-(3-(1- | |
| | Methylcyclopropyl)propyl)-1H-pyrano(2,3-d)pyrimidine-2,4,7(3H)-trione\|Erythrityl tetranitrate\|N- | |
| | (2,6-diphenylpyrimidin-4-yl)benzamide\|Chloroprocaine\|2-(4-Morpholinyl)-4H-1-benzopyran-4- | |
| | one\|2-(Morpholin-4-yl)-benzo[h]chromen-4-one\|3-(4-Morpholin-4-ylthieno[3,2-d]pyrimidin-2- | |
| | yl)phenol | |
| M3K1 | 2-(2-chloro-4-iodophenylamino)-N-cyclopropylmethoxy-3,4-difluorobenzamide\|3,4-dihydro-2H- | Inhibitor\|. |
| | pyrrol-5-amine\|Dacopafant\|1,4-Diamino-2,3-dicyano-1,4-bis(o- | |
| | aminophenylmercapto)butadiene\|Haegtftsdvssylegqaakeifawlvkgr\|2-Formamido-3-[3-[5-(2- | |
| | formamido-1-hydroxyethyl)-2-hydroxyphenyl]-4-hydroxyphenyl]-3-hydroxypropanoic | |
| | acid\|(6E,10E)-2,6,10-trimethyldodeca-2,6,10-triene\|Esketamine hydrochloride | |
| MK09 | TCS JNK 5a\|JNK Inhibitor VIII\|Jnk-IN-8\|U.S. Pat No. 9,765,058, Example 2-2-9\|1,3-Benzothiazol-2-yl(2-((2- | Inhibitor\|.\| |
| | (3-pyridinyl)ethyl)amino)-4-pyrimidinyl)acetonitrile\|Benzonitrile,3-(((5-(6-methyl-2-pyridinyl)-4-(6- | Activator |
| | quinoxalinyl)-1H-imidazol-2-yl)methyl)amino)-\|Acetamide,N-(4-amino-5-cyano-6-ethoxy-2- | |
| | pyridinyl)-IN-(4-amino-5-cyano-6-phenylpyridin-2-yl)acetamide\|N-(4-amino-6-butoxy-5- | |
| | cyanopyridin-2-yl)acetamide\|1,9-Pyrazoloanthrone\|1-(((Z)-(1-(2-aminothiazol-4-yl)-2-oxo-2- | |
| | (((3S,4R)-2-oxo-4-((2-oxooxazolidin-3-yl)methyl)-1-sulfoazetidin-3- | |
| | yl)amino)ethylidene)amino)oxy)cyclopropanecarboxylic acid\|Arbekacin\|Cefetamet Pivoxil HCl\|2- | |
| | (Cyclohexylamino)-8-(4-hydroxybutan-2-yl)pyrido[2,3-d]pyrimidin-7-one\|5-cyclohexyl-3-(1- | |
| | methylpyrazol-4-yl)-1H-pyrrolo[2,3-b]pyridine\|N-[(1S,2R)-2-[(1,3-Benzothiazol-2- | |
| | yl)amino]cyclopentyl]-2,6-dimethoxybenzamide\|Chlormezanone\|ethyl (7S)-11-oxo-14-(2- | |
| | trimethylsilylethynyl)-2,4,10-triazatetracyclo[10.4.0.02,6.07,10]hexadeca-1(16),3,5,12,14- | |
| | pentaene-5-carboxylate\|N,N-dimethyl-4-[3-(1H-pyrazol-4-yl)-1H-pyrrolo[2,3-b]pyridin-5- | |
| | yl]aniline\|Disodium hydrogen phosphate\|Methyl 3-[4-[5-(4-fluorophenyl)-2-pyrimidin-2-ylpyrazol- | |
| | 3-yl]piperidin-1-yl]-2-phenylpropanoate\|Thiolactomycin\|3-(thiophen-3-yl)-1H-indol-6-amine\|n-(4- | |
| | Chloro-3-(1h-1,2,4-triazol-5-yl)thiophen-2-yl)-2-(isoquinolin-5-yl)acetamide\|2-(4-chlorophenyl)-6- | |
| | phenyl-9H-purine\|3-Fluoro-5-(5-((2-methylthiazol-4-yl)ethynyl)pyridin-2-yl)benzonitrile\|N- | |
| | [(1S,2S)-2-[(6-Fluoro-1,3-benzothiazol-2-yl)amino]cyclopentyl]-N-methyl-2-(1H-pyrazol-1- | |
| | yl)benzamide\|Pregnan-20-one,3-hydroxy-3-(trifluoromethyl)-,(3alpha,5alpha)-\|Magnevist\|(1- | |
| | Methanesulfonyl-piperidin-4-yl)-{4-[4-(3-methyl-1h-pyrazol-4-yl)-benzotriazol-1-yl]-pyrimidin-2-yl}- | |
| | amine\|Cyclohexylamine\|CID 6509858\|Metamizole\|4-[(5-Piperazin-1-ylpyridin-2- | |
| | yl)amino]spiro[1,3,5,11-tetrazatricyclo[7.4.0.02,7]trideca-2,4,6,8-tetraene-13,1′-cyclohexane]-10- | |
| | one\|n-(4-Bromo-3-(1h-1,2,4-triazol-5-yl)thiophen-2-yl)-2-(2-oxo-6-(trifluoromethyl)quinolin-1(2h)- | |
| | yl)acetamide\|Jnk-IN-5\|3-[(E)-4-(dimethylamino)but-2-enoyl]amino]-N-[4-[4-(2- | |
| | phenylpyrazolo[1,5-a]pyridin-3-yl)pyrimidin-2-yl]amino]phenyl]benzamide | |
| ROCK2 | 2′-Monophosphoadenosine 5′-diphosphoribose\|Nalfurafine hydrochloride\|Aprosulate Sodium\|5- | Inhibitor\| |
| | [2-(Imidazol-1-yl)ethyl]quinoline\|H-1152 Glycyl Dihydrochloride\|1-(3-Hydroxybenzyl)-3-(4- | Modulator |
| | (pyridin-4-yl)thiazol-2-yl)urea\|N-(7-chloro-1-oxo-1,2-dihydroisoquinolin-6-yl)-2- | |
| | (cyclopropylamino)-2-phenylacetamide\|(3S,4R)-N-(7-chloro-1-oxo-1,2-dihydroisoquinolin-6-yl)-4- | |
| | (4-chlorophenyl)pyrrolidine-3-carboxamide\|(3R)-6-methoxy-N-[2-[(1-methylpiperidin-4- | |
| | yl)methoxy]-4-(1H-pyrazol-4-yl)phenyl]-1,2,3,4-tetrahydroisoquinoline-3-carboxamide\|2- | |
| | Cyclohexylacetic acidbiphenyl-3-yl ester\|N-[3-[2-(4-amino-1,2,5-oxadiazol-3-yl)-1- | |
| | ethylimidazo[4,5-c]pyridin-6-yl]oxyphenyl]-4-(2-morpholin-4-ylethoxy)benzamide\|Srtcx1003\|2-(3- | |
| | (4-((1H-indazol-5-yl)amino)quinazolin-2-yl)phenoxy)-N-isopropylacetamide\|4-(aminomethyl)-N- | |
| | (3-fluoropyridin-4-yl)-3-[3-[4-(2-oxooxolan-3-yl)sulfanylbenzoyl]amino]phenyl]benzamide\|N- | |
| | cyclobutyl-2-[3-[4-(1H-indazol-5-ylamino)-5,6-dimethylpyrimidin-2-yl]phenoxy]acetamide\|4-[2- | |
| | Chloro-3-(trifluoromethyl)benzoyl]-1-(2,4-difluorophenyl)piperazin-2-one\|N-(2-(3-(2- | |
| | (isopropylamino)ethoxy)phenyl)pyrimidin-4-yl)-1H-indazol-5-amine\|1-(4- | |
| | Hydroxybenzyl)imidazole\|6-[2-(5,5,8,8-Tetramethyl-6,7-dihydronaphthalen-2-yl)-1,3-dithiolan-2- | |
| | yl]naphthalene-2-carboxylic acid\|ethyl (2Z)-2-cyano-3-[(9-ethyl-9H-carbazol-3-yl)amino]prop-2- | |
| | enoate\|U.S. Pat No. 10,12,5144, Example 18\|1-(2-hydroxyethyl)-N-[3-[5-(1H-indazol-5-ylamino)-1,3,4- | |
| | thiadiazol-2-yl]phenyl]pyrazole-4-carboxamide\|N-(3-(ethylcarbamoyl) benzyl)-6H-isochromeno | |
| | [3,4-c]pyridine-8-carboxamide\|1-(1h-Indazol-5-Yl)-3-(2-Phenylethyl)urea\|N-[3-[(1- | |
| | ethylpyrrolidin-2-yl)methylcarbamoyl]phenyl]methyl]-6H-isochromeno[3,4-c]pyridine-8- | |
| | carboxamide\|N-[2-(5-methoxy-1,3-dihydroisoindol-2-yl)pyrimidin-4-yl]-1H-indazol-5-amine | |
| KPCA | #N/A\|3-Mercuri-4-Aminobenzenesulfonamide\|1-[2-(2,4,6-Trimethylphenyl)ethyl]pyrrolidine\|2-(2- | |
| | chloro-4-iodophenylamino)-N-cyclopropylmethoxy-3,4-difluorobenzamide\|Acteoside\|3-[1-[3- | |
| | (Dimethylamino)propyl]-5-methoxy-1H-indol-3-yl]-4-(1H-indol-3-yl)-1H-pyrrole-2,5-dione\|3-(1H- | |
| | Indol-3-yl)-4-phenylamino-pyrrole-2,5-dione\|Bisindolylmaleimide IV\|Prostratin\|(18S)-18- | |
| | (hydroxymethyl)-17-oxa-4,14,21-triazahexacyclo[19.6.1.17,14.02,6.08,13.022,27]nonacosa- | .\|Inhibitor\| |
| | 1(28),2(6),7(29),8,10,12,22,24,26-nonaene-3,5-dione\|Punicafolin\|BMS 813160-Bio-X\|(S)-4-(2- | Modulator |
| | (N-methylisoquinoline-5-sulfonamido)-3-oxo-3-(4-phenylpiperazin-1-yl)propyl)phenyl | |
| | isoquinoline-5-sulfonate\|4,5,6,7-Tetrabromobenzotriazole\|bisindolylmaleimide IX\|2-(1H-Indole-3- | |
| | yl)-3-phenylmaleimide\|1H-Pyrrole-2,5-dione,3,4-bis(4-methoxyphenyl)-\|3,4-diphenyl-1H-pyrrole- | |
| | 2,5-dione\|2-(4-Methoxyphenyl)-3-phenylmaleimide\|Leucosceptoside A\|Plantainoside | |
| | D\|Forsythiaside\|Calceolarioside B\|Calceolarioside A\|Dihexan-3-yl 5- | |
| | (hydroxymethyl)isophthalate\|Diheptan-3-yl 5-(hydroxymethyl)isophthalate\|1,2-Dioctanoyl-sn- | |
| | glycerol\|Cercosporamide\|Phenol,4,4′-(3-methyl-1-butenylidene)bis-\|(E)-3-(4-Hydroxyphenyl)- | |
| | 2,3-diphenylacrylonitrile\|4,4′-(Cycloheptylidenemethylene)diphenol\|2-(4-Hydroxyphenyl)-3,3- | |
| | diphenylacrylonitrile\|3,3-Bis(4-methoxyphenyl)-2-phenylpropenenitrile\|2,3,3- | |
| | Triphenylacrylonitrile\|3,3-Bis(4-hydroxyphenyl)-2-phenylprop-2-enenitrile\|4-Cyclopentyliden(4- | |
| | hydroxyphenyl)methylphenol\|Indiplon\|KT5720\|Cyclofenil diphenol\|3-(1-Methyl-1H-indol-3-yl)-4- | |
| | [6,7,8,9-tetrahydro-12-methyl-7beta,8alpha-(methaniminomethano)pyrido[1,2-a]indol]-10-yl]-1H- | |
| | pyrrole-2,5-dione\|2,2′:5′,2″-Terthiophene-4-carbaldehyde\|2,2′:5′,2″-Terthiophene-4,5″- | |
| | dicarbaldehyde\|2,2′:5′,2″-Terthiophene-4-methanol\|Sotrastaurin acetate\|Sodium | |
| | phenylbutyrate\|Balanol\|17-(Hydroxymethyl)-18-oxa-4,14,22- | |
| | triazahexacyclo[20.6.1.17,14.02,6.08,13.023,28]triaconta-1(29),2(6),7(30),8,10,12,23,25,27- | |
| | nonaene-3,5-dione\|(S)-3-(8-(Dimethylaminomethyl)-6,7,8,9-tetrahydropyrido(1,2-a)indol-10-yl)-4- | |
| | (1-methyl-3-indolyl)-1H-pyrrole-2,5-dione hydrochloride\|(+/−)-2-(4-Fluorophenyl)-7- | |
| | methylchroman-4-one | |
| ERBB4 | Clematomandshurica saponin B\|4-Methyl-N-(4-phenyl-1,3-thiazol-2-yl)benzamide\|(R,S)-1-(5- | .\|Inhibitor\| |
| | methyl-1H-indol-1-yl)propan-2-amine\|copper; acetic acid;(2S)-6-amino-2-[[(2S)-2-[(2- | Modulator\| |
| | aminoacetyl)amino]-3-(1H-imidazol-5-yl)propanoyl]amino]hexanoic acid\|Amphotericin b\|2-(8- | Antagonist |
| | Dimethylaminooctylthio)-6-isopropyl-3-pyridyl-2-thienyl ketone citrate\|Bermoprofen\|Naphtho[3,2- | |
| | f]quinoxaline-2,3,7,12-tetrone\|Para-Bromoamphetamine\|Carboxyatractyloside\|N-hydroxy-3-(4- | |
| | ((4-nitrobenzyloxyimino)methyl)phenyl)acrylamide\|CID 9799221\|N-(4-((3- | |
| | bromophenyl)amino)quinazolin-6-yl)but-2-ynamide\|EGFR/ErbB-2/ErbB-4 Inhibitor\|2-methyl-N-[2- | |
| | ({3-[2-(prop-2-enamido)acetamido]phenyl}amino)pyrimidin-5-yl]-5-[3- | |
| | (trifluoromethyl)benzamido]benzamide\|CID 73755245\|Urea,N-(1-((2-amino-4-pyridinyl)methyl)- | |
| | 1H-indol-4-yl)-N′-(5-bromo-2-methoxyphenyl)-\|Canertinib\|Idasanutlin\|Dacomitinib | |
| FAK2 | 7-butyl-6-(4-methoxyphenyl)-5H-pyrrolo[2,3-b]pyrazine | Inhibitor |
| NTRK1 | K-252a analogue\|5-Iodo-3-[(3,5-dibromo-4-hydroxyphenyl)methylene]-2-indolinone\|2- | Inhibitor\| |
| | Isopropoxy-12-(2-hydroxyethyl)-13,14-dihydronaphthol[2,1-a]pyrrolo[3,4-c]carbazole-5-one\|2- | Antagonist\| |
| | Methoxy-12-(2-hydroxyethyl)-13,14-dihydronaphthol[2,1-a]pyrrolo[3,4-c]carbazole-5- | Agonist |
| | one\|Sulfameter\|Fascaplysin\|6-Pyridin-3-yl-1,2,3,4-tetrahydronaphthalen-2-ol\|N-Cyclobutyl-N- | |
| | ((2,2-dimethyl-2H-pyrano[3,2-b]pyridin-6-yl)methyl)-3,4-dimethoxybenzenesulfonamide\|2-(3- | |
| | fluorophenyl)-1-(2-methyl-1-pentyl-1H-indol-3-yl)ethanone\|T-kinin\|N-[5-(4-tert-butylphenyl)-1,3,4- | |
| | thiadiazol-2-yl]-N-ethylpyridine-3-carboxamide\|(3S,4S,5S)-5-(Hydroxymethyl)piperidine-3,4- | |
| | diol\|2-Phenyl-5-(2-methylthiazol-4ylethynyl)pyridine\|5-[4-[2-(4-oxo-2H-1,3-benzoxazin-3- | |
| | yl)ethoxy]phenyl]methyl]-1,3-thiazolidine-2,4-dione\|Clothiapine\|Akt inhibitor VIII\|6-[6-(2-methyl- | |
| | 1H-pyrazol-2-ium-4-yl)-[1,2,4]triazolo[4,3-b]pyridazin-3-yl]sulfanyl]quinoline\|Unii- | |
| | VH3F3DR5X3\|3-((1H-imidazol-4-yl)methyl)pyridine\|1-(2-Methyl-1-pentyl-1H-indol-3-yl)-2-(4- | |
| | methylphenyl)ethanone\|2-[1,4]Diazepan-1-yl-6-nitro-quinoline\|3-[(3S)-3-ethyl-1-[8-[(3S)-3-ethyl- | |
| | 3-(3-hydroxyphenyl)azepan-1-yl]octyl]azepan-3-yl]phenol\|Isovelleral\|2-(3-Piperidin-1-yl-propyl)- | |
| | 3H-quinazolin-4-one\|3-(3-Adamantan-1-yl-ureido)-benzoic acid\|(5-Phenyl-thiazol-2-yl)-(4-(2- | |
| | pyrrolidin-1-yl-ethoxy)-phenyl)-amine\|8-O-(4-toluenesulfonyl)manzamine A\|(2S)-1-[(2R,3S)-3- | |
| | amino-2-hydroxy-5-methylhexanoyl]-N-[(2S)-1-amino-1-oxopropan-2-yl]pyrrolidine-2- | |
| | carboxamide\|Oritavancin\|Gusperimus trihydrochloride\|Tegaserod\|methyl 4-[4-[4-(1,1,3-trioxo- | |
| | 1,2-benzothiazol-2-yl)butyl]piperazin-1-yl]-1H-indole-2-carboxylate\|2-Phenyl-3-(piperidin-4-YL)- | |
| | 1H-indole\|(3R)-1-Acetyl-3-methylpiperidine\|Belizatinib\|(12Z)-9,10-Dihydroxyoctadec-12-enoic | |
| | acid\|(3S)-5-chlorospiro[2,4-dihydro-1H-naphthalene-3,4′-5H-1,3-oxazole]-2′-amine\|N-(5- | |
| | Benzyloxy-isoquinolin-1-yl)-guanidine\|2-[1-(2-Methylphenyl)pyrrol-3- | |
| | yl]ethanamine\|[3H]robalzotan\|(2S)-N-[(2S,3S)-1-amino-3-methyl-1-oxopentan-2-yl]-1-[(2R)-1- | |
| | [(2S)-2-[(2R)-2-[[(2S)-2-[(2S)-2-(benzoylamino)propanoyl]amino]propanoyl]amino]-3-(1H-indol- | |
| | 3-yl)propanoyl]amino]-3-phenylpropanoyl]pyrrolidine-2-carbonyl]pyrrolidine-2- | |
| | carboxamide\|cacospongionolide E\|Mirogabalin\|Hexylene | |
| | glycol\|Proguanil\|Aekkdegpyrmehfrwgsppkd[(2R)-1-[5-(3-tert-butylphenyl)-1H-indol-2- | |
| | yl]methyl]pyrrolidin-2-yl]methanol\|7-[3-(4-Acetyl-3-hydroxy-2-propylphenoxy)-2-hydroxypropoxy]- | |
| | 4-oxo-8-propyl-4H-1-benzopyran-2-carboxylic acid sodium salt\|SB 221284\|Para- | |
| | Bromoamphetamine\|N-(4-phenoxyphenyl)quinazolin-4-amine\|Idraparinux\|S-Sulfocysteine\|1- | |
| | Benzo[b]thiophen-2-yl-cycloheptylamine\|Levopropoxyphene napsylate anhydrous\|2-bromo-N-(2- | |
| | cyclopropyl-4-oxoquinazolin-3-yl)-5-methoxybenzamide\|Tirofiban\|Afegostat tartrate\|2- | |
| | Hydroxydodecanoic acid\|1,3-Dihydro-imidazo[4,5-b]quinolin-2-one\|1-Benzhydryl-4- | |
| | cyclopropylpiperidin-4-ol\|6-Methyl-4-(4-o-tolylpiperazin-1-yl)coumarin\|example 2 | |
| | [WO2013004676]\|N-{4″-[(trifluoromethyl)sulfonyl]-4-biphenyl}urea\|Tigapotide\|Amn082 | |
| | dihydrochloride\|Cyclohexylamine\|Promegestone\|5-(4-phenylphenyl)-2-undecan-6-yl-1H- | |
| | imidazole\|Glucagon-(1-6)\|9H-Pyrrolo[1′,2′:2,3]isoindolo[4,5,6-cd]indol-9-one, 10-acetyl- | |
| | 2,6,6a,7,11a,11b-hexahydro-11-hydroxy-7,7-dimethyl-,(6aR,11aS,11bR)-\|3-[4-[(2S)-2- | |
| | aminopropoxy]phenyl]-N-[(1R)-1-(3-fluorophenyl)ethyl]imidazo[1,2-b]pyridazin-6-amine\|5-{3-[6- | |
| | Methyl-4-(4-trifluoromethyl-phenyl)-pyridin-2-yl]-phenyl}-pyridin-2-ylamine\|Toluene\|3-[4-[(2S)- | |
| | azetidin-2-yl]methoxy]phenyl]-N-[(1R)-1-(3-fluorophenyl)ethyl]imidazo[1,2-b]pyridazin-6- | |
| | amine\|Cetraxate\|8-cyclopentyl-2,6-diphenyl-7H-purine\|3-amino-6-methyl-N-pyridin-2-ylpyrazine- | |
| | 2-carboxamide\|N-Benzyl-2-(toluene-4-sulfonylamino)-benzamide\|2-[6-(4-Chlorophenoxy)hexyl]- | |
| | 1-cyano-3-pyridin-4-ylguanidine\|4-Hexyl-5-methylbenzene-1,3-diol\|(3R,4S,6R)-2- | |
| | (hydroxymethyl)-6-methoxyoxane-3,4,5-triol\|Altiratinib\|Selitrectinib\|Tovinontrine\|Repotrectinib\|4- | |
| | hydroxy-6-nitro-3-phenylquinolin-2(1H)- | |
| | one\|Sakuranetin\|Pegcantratinib\|Tavilermide\|Entrectinib\|Larotrectinib\|U.S. Pat No. 10,323,022, | |
| | Example 152\|Pcmbs\|(6R,13S)-9-fluoro-13-hydroxy-2,11,15,19,20,23- | |
| | hexazapentacyclo[15.5.2.17,11.02,6.020,24]pentacosa-1(23),7,9,17(24),18,21-hexaene-16,25- | |
| | dione\|U.S. Pat No. 10,323,022, Example 610\|U.S. Pat No. 10,323,022, Example 613\|U.S. Pat No. 10,323,022, | |
| | Example 612\|1-(trans-4-Phenyl-1-(2-(trifluoromethoxy)ethyl)pyrrolidin-3-yl)-3-(2-phenyl-2,4,5,6- | |
| | tetrahydrocyclopenta[c]pyrazol-3-yl)urea\|Methyl 3-methoxy-2-((trans)-3-phenyl-4-(3-(2-phenyl- | |
| | 2,4,5,6-tetrahydrocyclopenta[c]pyrazol-3-yl)ureido)pyrrolidin-1-yl)propanoate\|U.S. Pat No. 10,323,022, | |
| | Example 130\|U.S. Pat No. 10,323,022, Example 317\|U.S. Pat No. 10,323,022, Example 312\|U.S. Pat No. 10,323,022, | |
| | Example 495\|U.S. Pat No. 10,323,022, Example 194\|Delorazepam\|U.S. Pat No. 10,323,022, Example 61\|2-Methoxy- | |
| | 4-(m-tolyloxy)benzonitrile\|N-cyclopropyl-5-((2R,4R)-2-(3-fluorophenyl)-4-hydroxypyrrolidin-1- | |
| | yl)pyrazolo[1,5-a]pyrimidine-3-carboxamide\|(R)-Larotrectinib\|(R)-N-cyclopropyl-5-(2-(5-fluoro-2- | |
| | (2-morpholinoethoxy)phenyl)pyrrolidin-1-yl)pyrazolo[1,5-a]pyrimidine-3-carboxamide\|N- | |
| | cyclobutyl-2-[3-[4-(1H-indazol-5-ylamino)-5,6-dimethylpyrimidin-2-yl]phenoxy]acetamide\|(3S)-N- | |
| | [5-[(2R)-2-(5-fluoro-2-methoxyphenyl)pyrrolidin-1-yl]pyrazolo[1,5-a]pyrimidin-3-yl]-3- | |
| | hydroxypiperidine-1-carboxamide\|(R,E)-3-(5-(2-(2,5-difluorophenyl)pyrrolidin-1-yl)pyrazolo[1,5- | |
| | a]pyrimidin-3-yl)-1-morpholinoprop-2-en-1-one\|2-Tert-butyl-5-(5-(2-(2,5-difluorophenyl)pyrrolidin- | |
| | 1-yl)pyrazolo[1,5-a]pyrimidin-3-yl)-1,3,4-thiadiazole\|5-(3-Chlorophenylethynyl)-5- | |
| | methyl[1,2,4]triazine\|2-(4,4-difluoropiperidin-1-yl)-6-methoxy-N-(1-propan-2-ylpiperidin-4-yl)-7-(3- | |
| | pyrrolidin-1-ylpropoxy)quinazolin-4-amine\|1-cyano-N-[5-[(2R)-2-(2,5-difluorophenyl)pyrrolidin-1- | |
| | yl]pyrazolo[1,5-a]pyrimidin-3-yl]cyclopropane-1-carboxamide\|Revizinone\|(S)-N-(6-((R)-2-(2,5- | |
| | difluorophenyl)pyrrolidin-1-yl)imidazo[1,2-b]pyridazin-3-yl)-3-hydroxypyrrolidine-1- | |
| | carboxamide\|4-[3-amino-4-(1,1,1-trifluoropropan-2-yloxy)-1H-indazol-6-yl]-2-(4-phenylpiperazin- | |
| | 1-yl)benzamide\|4-[3-amino-4-(oxan-4-yloxy)-1H-indazol-6-yl]-2-(4-phenylpiperazin-1- | |
| | yl)benzamide\|4-[3-amino-4-(oxan-4-yloxy)-1H-indazol-6-yl]-2-(4-cyclohexylpiperazin-1-yl)-N- | |
| | methylbenzamide\|(R)-N-(6-(2-(2,5-difluorophenyl)pyrrolidin-1-yl)imidazo[1,2-b]pyridazin-3-yl)-4- | |
| | (methylsulfonamido)benzamide\|N-cyclopropyl-5-((2R)-2-(3-(2,3-dihydroxypropoxy)-5- | |
| | fluorophenyl)pyrrolidin-1-yl)pyrazolo[1,5-a]pyrimidine-3-carboxamide\|U.S. Pat No. 10,323,022, Example | |
| | 441\|U.S. Pat No. 10,323,022, Example 511\|hTrkA-IN-1\|XX8E5WP7FS\|1-[3-(Dimethylamino)propyl]-3-[4- | |
| | [5,5-dimethyl-2,4-dioxo-3-[4-(trifluoromethylsulfanyl)phenyl]imidazolidin-1-yl]methyl]pyridin-2- | |
| | yl]urea\|1-[4,5-Dichloro-2-(3-methoxypropyl)phenyl]-3-[4-methyl-5-(1-methylpyrazol-4-yl)-2- | |
| | phenylpyrazol-3-yl]urea\|N-[4-(4-aminophenyl)sulfanyl-6-(4-methylpiperazin-1-yl)-1,3,5-triazin-2- | |
| | yl]-5-methyl-1,3-thiazol-2-amine\|CID 90168931\|5,5-Dimethyl-1-[2-(pyridin-2-ylamino)pyridin-4- | |
| | yl]methyl]-3-[4-(trifluoromethylsulfanyl)phenyl]imidazolidine-2,4-dione\|CID 73891136\|1-[4-[4-(4- | |
| | Methylpiperazin-1-yl)-6-[(5-methyl-1,3-thiazol-2-yl)amino]-1,3,5-triazin-2-yl]sulfanyl]phenyl]-3- | |
| | phenylurea\|[6-Cyclopropylmethoxy-5-(3,3-difluoro-azetidin-1-yl)-pyridin-2-yl]-(2,2-dimethyl- | |
| | pyrrolidin-1-yl)-methanone\|5-(3-Methoxy-4-((4-methoxybenzyl)oxy)benzyl)pyrimidine-2,4- | |
| | diamine\|1-(1′,4-dimethyl-1-phenyl-1H,1′H-[3,4′-bipyrazol]-5-yl)-3-(5-(methoxymethyl)-2- | |
| | (trifluoromethyl)benzyl)urea | |
| M4K2 | N-[4-[(4-ethylpiperazin-1-yl)methyl]-3-(trifluoromethyl)phenyl]-4-methyl-3-(1H-pyrrolo[2,3- | Inhibitor |
| | b]pyridin-4-yloxy)benzamide\|Senexin B | |
| PIM2 | XL888\|Benzo[c]-2,6-naphthyridin-5-amine,N-(2-chlorophenyl)-7-(1H-1,2,4-triazol-5-yl)-\|(R,Z)-5- | Inhibitor\| |
| | ((2-(3-aminopiperidin-1-yl)-[1,1′-biphenyl]-3-yl)methylene)thiazolidine-2,4-dione\|N-{4-[(1r,3s,5s)- | Modulator |
| | 3-Amino-5-Methylcyclohexyl]pyridin-3-Yl}-6-(2,6-Difluorophenyl)-5-Fluoropyridine-2- | |
| | Carboxamide | |
| VGFR3 | Sitravatinib\|N-(3-methylphenyl)-2-(4-pyridin-2-ylpiperidin-1-yl)acetamide\|(R)-2-amino-1-ethyl-7- | Inhibitor\| |
| | (3-hydroxy-4-methoxy-3-methylbut-1-yn-1-yl)-N-methyl-4-oxo-1,4-dihydro-1,8-naphthyridine-3- | Modulator |
| | carboxamide\|3-[(4-chlorophenyl)-(1H-imidazol-2-yl)methylidene]-5-[(1-ethylpiperidin-4-yl)amino]- | |
| | 1H-indol-2-one\|Pyrazolo[1,5-a]pyrimidine 1a\|N-[2,4-dichlorobenzoyl]phenylsulfonamide\|4- | |
| | Chloro-N-(2-chlorobenzoyl)benzenesulfonamide\|3-chloro-N-(4- | |
| | chlorophenyl)sulfonylbenzamide\|4-Chloro-N-[(4-chlorophenyl)sulfonyl]benzamide\|N,5-diphenyl- | |
| | 1,3-oxazol-2-amine\|2-[(5-Phenyl-1,3-oxazol-2-yl)amino]benzonitrile\|N-(2-methoxyphenyl)-5- | |
| | phenyl-1,3-oxazol-2-amine\|3-[(5-Phenyl-1,3-oxazol-2-yl)amino]benzonitrile\|3-(5-Phenyl-oxazol-2- | |
| | ylamino)phenylmethanol\|N-(4-chlorobenzenesulfonyl)-2-methylbenzamide\|3-bromo-N-(4- | |
| | chlorophenyl)sulfonylbenzamide\|N-(4-chlorophenyl)sulfonyl-4-nitrobenzamide\|N-(3- | |
| | methylphenyl)-5-phenyl-1,3-oxazol-2-amine\|4-[(5-Phenyl-1,3-oxazol-2- | |
| | yl)amino]benzenesulfonamide\|N-(4-chloro-2-fluorophenyl)-6,7-dimethoxyquinazolin-4-amine\|2- | |
| | Isopropoxy-12-(2-hydroxyethyl)-13,14-dihydronaphthol[2,1-a]pyrrolo[3,4-c]carbazole-5-one\|4- | |
| | Oxobutanoate\|CID 73755245\|MK- | |
| | 2461\|Vatalanib\|Famitinib\|Sulfatinib\|pentadecasodium;[(2R,3R,4S,5S,6R)-6-[(2R,3S,4S,5R,6R)- | |
| | 2-[(2R,3S,4S,5R,6R)-2-[(2R,3R,4S,5S,6R)-3,5-disulfonatooxy-2-(sulfonatooxymethyl)-6- | |
| | [(2R,3S,4R,5R,6R)-2,4,5-trisulfonatooxy-6-(sulfonatooxymethyl)oxan-3-yl]oxyoxan-4-yl]oxy-3,5- | |
| | disulfonatooxy-6-(sulfonatooxymethyl)oxan-4-yl]oxy-3,5-disulfonatooxy-6- | |
| | (sulfonatooxymethyl)oxan-4-yl]oxy-3,4,5-trisulfonatooxyoxan-2-yl]methyl phosphate\|N-(3,5- | |
| | dichlorophenyl)-1H-indazole-5-carboxamide\|Linifanib\|Lucitanib\|Fruquintinib\|Tivozanib\|Emetine | |
| PAK2 | 4-N-(5-cyclopropyl-1H-pyrazol-3-yl)-2-N-[(6-fluoro-1H-indazol-4-yl)methyl]-2-N-(2- | Inhibitor |
| | methoxyethyl)pyrimidine-2,4-diamine | |
| PDPK1 | example 41 [WO2012028563]\|U.S. Pat No. 8,569,313, Inhibitor 14\|6-[2-(1H-indol-6-yl)ethyl]pyridin-2- | Binder\|.\| |
| | amine\|15(R)-15-methyl Prostaglandin D2\|[3H]N-methyl-AZ10419369\|Halobetasol propionate\|3- | Inhibitor\| |
| | sec-Butoxy-9H-beta-carboline\|2-Pyrimidinamine,N-ethyl-4-(4-(3-fluorophenyl)-3,6-dihydro- | Modulator |
| | 1(2H)-pyridinyl)-6-methyl-N-(4-(1-methylethyl)-2-(methylthio)phenyl)-\|4-(6-Hydroxy-7- | |
| | tricyclo[3.3.1.13,7]dec-1-yl-2-naphthalenyl)benzoic acid\|4-(5-Chloro-1h,3h- | |
| | Benzo[de]isochromen-6-Yl)-6-(Methylsulfanyl)-1,3,5-Triazin-2-Amine\|Raluridine\|1-hexadecyl-1H- | |
| | indole-2,3-dione\|Permethrin\|2,2′:5′,2″-Terthiophene-4-carbaldehyde\|[6-(4-Methylpiperazin-1- | |
| | yl)benzo[b][1,4]benzoxazepin-8-yl]trifluoromethanesulfonate\|Pentetate calcium trisodium\|1,3,5- | |
| | Triphenylimidazoline-2,4-dione\|CID 9690114\|Tricaprylin\|Umeclidinium\|Indantadol | |
| | hydrochloride\|(Z)-3-((1H-pyrrol-2-yl)methylene)indolin-2-one\|(3Z)-3-[(3,5-dimethyl-1H-pyrrol-2- | |
| | yl)methylidene]-5-hydroxy-2,3-dihydro-1H-indol-2-one\|[(3Z)-2-oxo-3-(1H-pyrrol-2-ylmethylidene)- | |
| | 2,3-dihydro-1H-indol-5-yl]urea\|Urea,(2,3-dihydro-2-oxo-3-(1-(1H-pyrrol-2-yl)ethylidene)-1H- | |
| | indol-5-yl)-\|06-Cyclohexylmethoxy-2-(4′-sulphamoylanilino)purine\|(3S,6R)-1-[6-(3-Amino-1H- | |
| | indazol-6-yl)-2-(methylamino)-4-pyrimidinyl]-N-cyclohexyl-6-methyl-3-piperidinecarboxamide\|2- | |
| | (Cycloheptylamino)-2-oxoethyl 2-aminonicotinate\|4-dodecyl-N-(1,3,4-thiadiazol-2- | |
| | yl)benzenesulfonamide\|(2s)-1-{[6-Furan-3-Yl-5-(3-Methyl-2h-Indazol-5-Yl)pyridin-3-Yl]oxy}-3- | |
| | (1h-Indol-3-Yl)propan-2-Amine\|N-[3-[5-Iodo-4-[3-[(2-thienylcarbonyl)amino]propyl]amino]-2- | |
| | pyrimidinyl]amino]phenyl]-1-pyrrolidinecarboxamide\|N-[3-[5-Bromo-4-[2-(1H-imidazol-5- | |
| | YL)ethyl]amino]-2-pyrimidinyl]amino]phenyl]-1-pyrrolidinecarboxamide\|Phosphoserine\|PDK1 | |
| | inhibitor AR-12 | |
| LRRK2 | JAK3 inhibitor VI mesylate\|3-[4-(morpholin-4-yl)-7H-pyrrolo[2,3-d]pyrimidin-5-yl]benzonitrile\|5-(1- | Inhibitor |
| | methyl-1H-pyrazol-4-yl)-4-(morpholin-4-yl)-7H-pyrrolo[2,3-d]pyrimidine\|3-(1H-indol-5-yl)-5-(4-((4- | |
| | methylpiperazin-1-yl)methyl)phenyl)-1H-pyrrolo[2,3-b]pyridine\|[4-[4-(Ethylamino)-5- | |
| | (trifluoromethyl)pyrimidin-2-yl]amino]-2-fluoro-5-methoxyphenyl]-morpholin-4-ylmethanone\|2-N- | |
| | [5-chloro-1-[(3S,4S)-3-fluoro-1-(oxetan-3-yl)piperidin-4-yl]pyrazol-4-yl]-4-N-methyl-5- | |
| | (trifluoromethyl)pyrimidine-2,4-diamine\|leukotriene D4\|Saframycin A\|(3Z)-6-(4-Hydroxy-3- | |
| | methoxyphenyl)-3-(1H-pyrrol-2-ylmethylene)-1,3-dihydro-2H-indol-2-one\|Enzalutamide\|4-[1-[2- | |
| | [(3S)-1-(cyclopropylmethyl)-3-(3,4-dichlorophenyl)-6-oxopiperidin-3-yl]ethyl]azetidin-3- | |
| | yl]piperazine-1-sulfonamide\|N,N′-Bis(5-acenaphthyl)guanidine\|(4S)-2-(2-aminoethoxymethyl)-3- | |
| | N-[3-(4,4-diphenylpiperidin-1-yl)propyl]-6-ethyl-4-(4-nitrophenyl)-1,4-dihydropyridine-3,5- | |
| | dicarboxamide\|Deoxy-bigchap\|8-Cyclobutylmethoxy-quinolin-2-ylamine\|Volinanserin C- | |
| | 11\|Minamestane\|Isoflurane\|(S)-(+)-tert-Butyl 2-(4-(4-chlorophenyl)-2,3,9-trimethyl-6H-thieno(3,2- | |
| | f)(1,2,4)triazolo(4,3-a)(1,4)diazepin-6-yl)acetate\|VEGF Receptor 2 Kinase Inhibitor II\|1-(1,2- | |
| | Diphenylethyl)piperazine\|3-Fluoro-4-hydrazino-benzenesulfonamide\|Latrepirdine\|3-{[3-Oxo-2-(2- | |
| | piperidin-4-yl-ethyl)-2,3-dihydro-1H-isoindole-5-carbonyl]-amino}-propionic acid\|N-hydroxy-4- | |
| | (naphthalen-1-yl)benzamide\|Tepoxalin\|3,6-Dimethyl-N-(Pentan-3-Yl)-2-(2,4,6- | |
| | Trimethylphenoxy)pyridin-4-Amine\|Curcumin\|1-(1-{[1-(3a,7a-dihydro-2H-1,3-benzodioxol-5- | |
| | ylmethyl)-1H-imidazol-5-yl]methyl}-4-(naphthalen-1-yl)-1H-pyrrole-3-carbonyl)-4- | |
| | methylpiperazine\|6-Amino-2-(3-bromophenylthio)benzenecarbonitrile\|8-(4-bromophenyl)-N- | |
| | hydroxy-8-oxooctanamide\|3-[(1Z,4Z,10Z,14Z)-18-(2-carboxyethyl)-7,12-bis(ethenyl)-3,8,13,17- | |
| | tetramethylporphyrin-21,22,23,24-tetraid-2-yl]propanoic acid; tin(4+)\|Aspartyl-alanyl- | |
| | diketopiperazine\|Cyclobenzaprine hydrochloride\|Uridine diphosphate glucuronic acid\|5-chloro-N- | |
| | [(E)-[3-(4-fluorophenyl)-4-oxo-1,3-thiazolidin-2-ylidene]amino]thiophene-2- | |
| | carboxamide\|Tallimustine hydrochloride\|N-hydroxy-4-(2-oleamidoethyl)benzamide\|1- | |
| | (Naphthalen-2-yl)-2-(pyrrolidin-1-yl)pentan-1-one\|Thiophosphoric acid (E)-dodec-9-enyl ester | |
| OXSR1 | 2-(2-Aminopyrimidin-4-yl)-4-(2,4-dichlorophenyl)-1,3-thiazole-5-carboxamide | Inhibitor |
| CDK12 | (R,E)-N-(4-(3-((5-chloro-4-(1H-indol-3-yl)pyrimidin-2-yl)amino)piperidine-1-carbonyl)phenyl)-4- | Inhibitor |
| | (dimethylamino)but-2-enamide\|1-Propanesulfonamide,3,3,3-trifluoro-N-[trans-4-[3-[(2S)-2- | |
| | methyl-4-morpholinyl]-1H-pyrazolo[3,4-d]pyrimidin-6-yl]amino]cyclohexyl]- | |
| JAK2 | Ethyl 6-bromo-4-hydroxyquinoline-3-carboxylate\|3-amino-5-[(2S)-2-amino-3-[(2S)-1-[(2S)-1- | Inhibitor\| |
| | [[(2R,3R)-4-(3-carboxyanilino)-3-hydroxy-4-oxo-1-phenylsulfanylbutan-2-yl]amino]-4-methyl-1- | Modulator |
| | oxopentan-2-yl]amino]-3-methyl-1-oxobutan-2-yl]amino]-3-oxopropyl]carbamoyl]benzoic acid\|11- | |
| | Keto-beta-boswellic acid\|4-[2-[(4-Bromophenyl)methylsulfanyl]acetyl]benzoic acid\|[(4- | |
| | Chlorophenyl)-sulfanylmethyl]phosphonic acid\|Pyridone 6\|2-Bromo-4-[(6,7-dimethoxyquinazolin- | |
| | 4-yl)amino]phenol\|2-N-((1S)-1-(5-Fluoropyrimidin-2-yl)ethyl)-4-N-(1-methylimidazol-4-yl)-7H- | |
| | pyrrolo(2,3-d)pyrimidine-2,4-diamine\|(S)-5-fluoro-2-(1-(5-fluoropyridin-2-yl)ethylamino)-6-(5- | |
| | methyl-1H-pyrazol-3-ylamino)nicotinonitrile\|7-(3,5-Difluoro-4-(morpholinomethyl)phenyl)-N-(4- | |
| | (cis-3,5-dimethylpiperazin-1-yl)phenyl)-7H-pyrrolo(2,3-d)pyrimidin-2-amine\|AZ-960\|5-phenyl-1H- | |
| | indazol-3-amine\|Atropisomer 1\|Thiram\|1,2,3,4,5,6-Hexabromocyclohexane\|Tyrphostin B42\|CID | |
| | 16750271\|Neoandrographolide\|(4-fluorophenyl)(4-((5-methyl-1H-pyrazol-3-yl)amino)quinazolin- | |
| | 2-yl)methanol\|Ilginatinib\|Itacitinib\|Bms-911543\|Gandotinib\|Cerdulatinib\|(S)-5-chloro-N2-(1-(5- | |
| | fluoropyrimidin-2-yl)ethyl)-N4-(5-methyl-1H-pyrazol-3-yl)pyrimidine-2,4- | |
| | diamine\|Deuruxolitinib\|(S)-N-(4-(2-((4-morpholinophenyl)amino)pyrimidin-4-yl)phenyl)pyrrolidine- | |
| | 2-carboxamide\|Givinostat\|Pacritinib\|Momelotinib\|Fedratinib\|Ruxolitinib\|Baricitinib\|N~4~-(Acridin- | |
| | 9-yl)-N~1~,N~1~-diethylbutane-1,4-diamine\|Cdk7-IN-2\|3-(2-(Pyridin-4-yl)ethyl)-1H-indole\|1-(4- | |
| | Iodophenyl)-2-pyrrolidin-1-yl-pentan-1-one\|13,16,19,22-Tetraoxa-3,6- | |
| | diazatricyclo[21.3.1.18,12]octacosa-1(27),2,6,8(28),9,11,23,25-octaene-27,28-diol\|Balapiravir\|1- | |
| | (5-Bromo-2-pyridyl)-3-[2-(3-fluorophenyl)ethyl]thiourea\|Gliclazide\|(5-(3-methoxyphenyl)-1-pentyl- | |
| | 1H-pyrrol-3-yl)(naphthalen-1-yl)methanone\|(3-(4-(6-Oxa-2-azaspiro[3.4]octan-2-ylmethyl)-3- | |
| | methylphenoxy)azetidin-1-yl)(5-phenyl-1,3,4-oxadiazol-2-yl)methanone\|[(4R,4aR,7S,7aR,12bS)- | |
| | 9-acetyloxy-3-methyl-1,2,3,4,4a,7,7a,13-octahydro-4,12-methanobenzofuro[3,2-e]isoquinolin-3- | |
| | ium-7-yl]sulfate\|Bifeprunox\|Fluspirilene\|2-Ethyl-9-methyl-12-thia-2,4,9,17- | |
| | tetrazatetracyclo[9.7.0.03,8.013,18]octadeca-1(11),3(8),4,6,13(18),14,16-heptaene-10-thione\|1- | |
| | Cyclohexyl-4-[3-(5-methoxy-1,2,3,4-tetrahydronaphthalen-1-yl)propyl]piperazine\|Enadoline\|7-(3- | |
| | Methoxyphenyl)-2-methylquinoline\|4-[3-(2-Fluorophenoxy)phenyl]-1-phosphonatobutane-1- | |
| | sulfonate\|N,N′-Bis(3-(ethylamino)propyl)-1,7-heptanediamine\|Bucladesine sodium\|5-Butyl-8- | |
| | phenyl-3H-[1,2,4]triazolo[5,1-i]purine\|Crenigacestat\|(S)-5-(3-Fluorophenyl)-N-(piperidin-3-yl)-3- | |
| | ureidothiophene-2-carboxamide\|Loviride\|8-Bromo-3-methyl-5-phenyl-2,3,4,5-tetrahydro-1H- | |
| | benzo[d]azepin-7-ol\|Moxidectin\|2-[4-(3-Methyl-2-thienyl)phenyl]propionic | |
| | acid\|Indiplon\|Rilzabrutinib\|Unii-zsa4DW7Idj\|N-(5-(3,5-difluorophenylthio)-1-H-pyrazolo[3,4- | |
| | b]pyridine-3-yl)-4-(4-methylpiperazine-1-yl)-2-(tetrahydro-2H-pyran-4-ylamino)benzamide\|N-(7- | |
| | chloro-1-oxo-1,2-dihydroisoquinolin-6-yl)-2-(cyclopropylamino)-2-phenylacetamide\|5,8-dichloro- | |
| | 7-[1-(2-hydroxyacetyl)piperidin-4-yl]-methoxymethyl]-2-[(4-methoxy-6-methyl-2-oxo-1H-pyridin- | |
| | 3-yl)methyl]-3,4-dihydroisoquinolin-1-one\|H-Gly-Gly-Thr-Thr-Gly-Gly-Thr-Gly-Thr-Gly-Gly-Thr- | |
| | Thr-Gly-Gly-OH\|Nicotinamide riboside\|Acetic acid 2-heptylselanyl-phenyl | |
| | ester\|Kifunensine\|U.S. Pat No. 10,150,740, Example 9\|4-(5-(4-chlorophenyl)-4-(pyridin-3-yl)-1H-imidazol-2- | |
| | yl)phenol\|6-(4-Deoxy-4-((2E,4E)-tetradecadienoylglycyl)amino-L-glycero-b-L- | |
| | mannoheptopyranosyl)amino-9H-purine\|4-[(Z)-1-(1-Naphthyl)-1-propenyl]-1H-imidazole\|1-[5- | |
| | Ethyl-2-hydroxy-4-[6-methyl-6-(1H-tetrazol-5-YL)heptyl]oxy]phenyl]ethanone\|7 alpha,27- | |
| | Dihydroxycholesterol\|8-(1,3-oxazol-5-yl)-N-phenyl-6-(trifluoromethyl)imidazo[1,2-a]pyridine-3- | |
| | carboxamide\|2-(3-Chloro-phenyl)-1H-[1,8]naphthyridin-4-one\|N-[4-chloro-3-[2-[4-(4- | |
| | methylpiperazin-1-yl)anilino]-5-oxo-7,8-dihydropyrido[4,3-d]pyrimidin-6-yl]phenyl]-3- | |
| | (trifluoromethyl)benzamide\|(R)-N2-{4-[(3-chlorobenzyl)oxy]benzyl}alaninamide\|Topaquinone\|3- | |
| | (2-Hydroxyethylcarbamoyl)pyrido[1,2-a]indole-10-carboxylic acid\|1-(3-carbamimidoylphenyl)-3- | |
| | methyl-N-[4-(2-sulfamoylphenyl)phenyl]-1H-pyrazole-5-carboxamide\|N-[4-chloro-3-[(2-methyl-3- | |
| | oxocyclopenten-1-yl)amino]phenyl]-1-phenylmethanesulfonamide\|Cyclofenil\|Tyr-Pro-Phe-Hfe- | |
| | NH2\|Rhein\|N-Cyclopropyl-5-[2-Methyl-5-(Trifluoromethoxy)-1h-Benzimidazol-1-Yl]thiophene-2- | |
| | Carboxamide\|7,3′,4′-Trihydroxyflavone\|deamino-Cys(1)-nTyr-D-xille-D-Gln-D-Asn-D-Cys(1)-D- | |
| | Pro-Lys(Unk)-Unk\|Urea,N-cyclopropyl-N′-(3-((1,2-dihydro-2-oxo-6-quinolinyl)oxy)propyl)-N- | |
| | ((1R,2R)-2-hydroxycyclohexyl)-\|2H-Benzofuro(3,2-g)-1-benzopyran-2-one,4-methyl-\|ENS-163 | |
| | phosphate\|Dasantafil\|1-(2-Methoxyphenyl)-4-propylpiperazine\|(2S)-N-dodecyl-1-[(2S)- | |
| | pyrrolidine-2-carbonyl]pyrrolidine-2-carboxamide\|2-Methoxy-N-n-propylnorapomorphine\|2- | |
| | Hydrazinylbenzenesulfonamide\|Tubercidin\|Thiorphan\|(R)-N-[(1S,2R)-2-Hydroxyindan-1-yl]-3- | |
| | [(2S,3S)-3-(4-methoxyphenoxyacetyl)amino-2-hydroxy-4-phenylbutanoyl]-5,5-dimethyl-1,3- | |
| | thiazolidine-4-carboxamide\|1-(4-Bromooxazol-2-yl)-7-phenylheptan-1-one\|1-(3-Acetyloxy-17-(1- | |
| | oxobutoxy)-2-(1-piperidinyl)-androstan-16-yl)-1- | |
| | methylpiperidinium\|FIII32\|Tetramethylcurcumin\|Sardomozide\|[(2S)-1-[(2R)-2-boronopyrrolidin-1- | |
| | yl]-1-oxopropan-2-yl]azanium\|6H-(1,2,4)Triazolo(4,3-a)(1,5)benzodiazepine-6-acetamide,4,5- | |
| | dihydro-4-(1H-indol-3-ylmethyl)-N-(1-methylethyl)-5-oxo-1-phenyl-N-(phenylmethyl)-, (S)- | |
| EPHA6 | (2S)-2-[(4R)-4-[(3R,5R,8R,9S,10S,13R,14S,17R)-3-hydroxy-10,13-dimethyl- | Inhibitor |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid | |
| CSK | N-[4-[(4-ethylpiperazin-1-yl)methyl]-3-(trifluoromethyl)phenyl]-4-methyl-3-(1H-pyrrolo[2,3- | Inhibitor |
| | b]pyridin-4-yloxy)benzamide\|2-PropenaMide,N-[cis-4-[4-aMino-3-(4-phenoxyphenyl)-1H- | |
| | pyrazolo[3,4-d]pyriMidin-1-yl]cyclohexyl]- | |
| LIMK2 | (S)-N-(3-bromophenyl)-N′-cyano-2-methyl-4-(5-methyl-7H-pyrrolo[2,3-d]pyrimidin-4- | Inhibitor |
| | yl)piperazine-1-carboximidamide\|3-[(cyanoamino)-[(2S)-2-methyl-4-(5-methyl-7H-pyrrolo[2,3- | |
| | d]pyrimidin-4-yl)piperazin-1-yl]methylidene]amino]-N-(2-hydroxyethyl)benzamide\|1-[5-[6-(2,6- | |
| | Dimethyl-4-pentan-3-yloxyphenyl)-2-pyrazin-2-ylpyrimidin-4-yl]-1,3-thiazol-2-yl]-3-methylurea\|1- | |
| | Ethyl-3-[5-[6-(4-methoxy-2,6-dimethylphenyl)-2-pyrazin-2-ylpyrimidin-4-yl]-1,3-thiazol-2- | |
| | yl]urea\|N-[5-[6-(2,6-dichloro-4-methoxyphenyl)-2-pyrazin-2-ylpyrimidin-4-yl]-1,3-thiazol-2- | |
| | yl]acetamide | |
| DDR1 | N-(3-chlorophenyl)-4-methyl-3-(2-pyrazolo[1,5-a]pyrimidin-6-ylethynyl)benzamide\|N-(5-Chloro-2- | Inhibitor |
| | ethanesulfonyl-benzyl)-3-trifluoromethyl-benzamide\|3-bromo-N-[(5-chloro-2- | |
| | ethylsulfonylphenyl)methyl]-5-(trifluoromethyl)benzamide\|N-[1-(4-acetylanilino)-6- | |
| | methylisoquinolin-5-yl]-4-aminothieno[3,2-d]pyrimidine-7-carboxamide\|N-(5-Chloro-2- | |
| | ethanesulfonyl-benzyl)-4-(4-methyl-piperazin-1-ylmethyl)-3-trifluoromethyl-benzamide\|8-chloro- | |
| | 3-[(5-chloro-2-ethylsulfonylphenyl)methyl]-7-(piperazin-1-ylmethyl)-6-(trifluoromethyl)-8H- | |
| | quinazoline-2,4-dione\|7-((R)-3-Amino-piperidin-1-ylmethyl)-8-chloro-3-(5-chloro-2- | |
| | ethanesulfonyl-benzyl)-6-trifluoromethyl-1H-quinazoline-2,4-dione\|3-{2- | |
| | [(cyclopropylcarbonyl)amino][1,3]thiazolo[5,4-b]pyridin-5-yl}-N-{4-[(4-ethylpiperazin-1-yl)methyl]- | |
| | 3-(trifluoromethyl)phenyl}benzamide\|8-Chloro-3-[(5-chloro-2-ethylsulfonylphenyl)methyl]-7- | |
| | [(3S)-3-(2-hydroxyethylamino)piperidin-1-yl]methyl]-6-(trifluoromethyl)-1H-quinazoline-2,4-dione | |
| BRSK2 | 3-[(2R)-1-(dimethylamino)propan-2-yl]oxy-5-[4-methoxy-5-(1-methylpyrazol-4-yl)pyridin-2- | Inhibitor |
| | yl]amino]pyrazine-2-carbonitrile | |
| CDK8 | #N/A\|4-(4-Fluorophenyl)-2-(4-hydroxyphenyl)-5-(4-pyridyl)-1H-imidazole\|8-(3-chloro-5-(4-(1- | Inhibitor |
| | methyl-1H-pyrazol-4-yl)phenyl)pyridin-4-yl)-2,8-diazaspiro[4.5]decan-1-one\|SEL120-34A HCl\|7- | |
| | Oxa-10,14,19,21,22-pentazatetracyclo[13.5.2.12,6.018,21]tricosa-1(20),2(23),3,5,15(22),16,18- | |
| | heptaene\|5,6,7-Tribromo-2-piperazin-1-yl-1,3-diazatricyclo[6.3.1.04,12]dodeca-2,4,6,8(12)- | |
| | tetraene | |
| SIK1 | 1-(2,4-Dimethoxyphenyl)-3-(2,6-dimethylphenyl)-1-[6-[4-(4-methylpiperazin-1- | Inhibitor |
| | yl)anilino]pyrimidin-4-yl]urea\|N-[(1S)-1-(5-fluoropyridin-2-yl)ethyl]-3-(3-propan-2-yloxy-1H- | |
| | pyrazol-5-yl)imidazo[4,5-b]pyridin-5-amine | |
| M3K3 | 4R-Fluoro-N6-ethanimidoyl-L-lysine\|Choline salicylate\|(2-Hydroxyimino-2-phenyl-ethyl)-carbamic | .\|Inhibitor |
| | acid tert-butyl ester \|N6-[(3,4,5-trimethoxyphenyl)methyl]quinazoline-2,4,6-triamine\|Contortumine | |
| | hydrochloride\|Hydroxyethyl starch\|N-[2-[5-(2-hydroxyethoxy)-1H-indol-3-yl]ethyl]acetamide\|2- | |
| | [(1s,2s)-2-Carboxycyclopropyl]-3-(9h-Xanthen-9-Yl)-D-Alanine\|Oxiracetam\|Examorelin\|1-[5- | |
| | chloro-2-(trifluoromethyl)phenyl]methyl]-7-(2-piperazin-1-ylpyridin-4-yl)-3,4-dihydro-2H- | |
| | pyrido[2,3-b]pyrazine | |
| DDR2 | 4-amino-N-[1-(4-chloroanilino)-6-methylisoquinolin-5-yl]thieno[3,2-d]pyrimidine-7-carboxamide\|4- | Inhibitor |
| | amino-N-[1-(3-chloroanilino)-6-methylisoquinolin-5-yl]thieno[3,2-d]pyrimidine-7-carboxamide\|4- | |
| | amino-N-[1-(3-chloro-4-fluoroanilino)-6-methylisoquinolin-5-yl]thieno[3,2-d]pyrimidine-7- | |
| | carboxamide | |
| TRPM7 | [(2R)-3-[hydroxy-[(1R,2R,3S,4R,5R,6S)-2,3,6-trihydroxy-4,5- | Activator\| |
| | diphosphonooxycyclohexyl]oxyphosphoryl]oxy-2-[(Z)-octadec-9-enoyl]oxypropyl] (Z)-octadec-9- | Blocker |
| | enoate\|waixenicin A\|2-Aminoethyl diphenylborinate\|Carvacrol | (channel |
| | | blocker) |
| ACVR1 | 1-(4-(6-Methyl-5-(3,4,5-trimethoxyphenyl)pyridin-3-yl)phenyl)piperazine\|5-[6-(4- | Inhibitor |
| | Methoxyphenyl)pyrazolo[1,5-a]pyrimidin-3-yl]quinoline\|4-(4-(3-(1H-Pyrazol-4-yl)pyrazolo[1,5- | |
| | a]pyrimidin-6-yl)phenyl)morpholine\|4-[4-(3-Quinolin-4-ylpyrazolo[1,5-a]pyrimidin-6- | |
| | yl)phenyl]morpholine\|4-[6-[4-(4-Methylpiperazin-1-yl)phenyl]pyrazolo[1,5-a]pyrimidin-3- | |
| | yl]quinoline\|Itacnosertib\|N,N,N′,N′-Tetramethyl-p-phenylenediamine | |
| | dihydrochloride\|Kifunensine\|2-Ethyl-9-methyl-12-thia-2,4,9,17- | |
| | tetrazatetracyclo[9.7.0.03,8.013,18]octadeca-1(11),3(8),4,6,13(18),14,16-heptaene-10-thione | |
| M3K11 | 3-(1H-indol-5-yl)-5-(4-((4-methylpiperazin-1-yl)methyl)phenyl)-1H-pyrrolo[2,3-b]pyridine\|3-(1H- | Inhibitor |
| | indol-5-yl)-5-(3,4,5-trimethoxyphenyl)-1H-pyrrolo[2,3-b]pyridine\|3′-Deoxy-3′-(1,2,3-triazol-1- | |
| | yl)thymidine\|2-Isopropoxy-12-(2-hydroxyethyl)-13,14-dihydronaphthol[2,1-a]pyrrolo[3,4- | |
| | c]carbazole-5-one\|2-Methoxy-12-(2-hydroxyethyl)-13,14-dihydronaphthol[2,1-a]pyrrolo[3,4- | |
| | c]carbazole-5-one | |
| LCK | 2-(2-chloro-4-iodophenylamino)-N-cyclopropylmethoxy-3,4-difluorobenzamide\|4-cyano-N-(5-(1- | Inhibitor |
| | (2-(dimethylamino)acetyl)piperidin-4-yl)-2′,3′,4′,5′-tetrahydro-[1,1′-biphenyl]-2-yl)-1H-imidazole-2- | |
| | carboxamide\|Lck Inhibitor\|2,6-Dimethylphenyl (2,4-dimethoxyphenyl)(2-((4-(4-methylpiperazin-1- | |
| | yl)phenyl)amino)pyrimidin-4-yl)carbamate\|[2-chloro-4-[6-cyclopropyl-3-(1H-pyrazol-4- | |
| | yl)imidazo[1,2-a]pyrazin-8-yl]amino]phenyl]-(2,2-dimethylpiperazin-1-yl)methanone\|N-(2-chloro- | |
| | 6-methylphenyl)-2-[(2,6-dimethylpyrimidin-4-yl)amino]-1,3-thiazole-5-carboxamide\|3-[ 4-(5- | |
| | Hydroxy-2-methylanilino)pyrimidin-2-yl]amino]benzenesulfonamide\|N-phenethyl-5-phenyl-6-(4- | |
| | (2-(pyrrolidin-1-yl)ethoxy)phenyl)furo[2,3-d]pyrimidin-4-amine\|Phosphoaminophosphonic acid- | |
| | adenylate ester\|N-(4-phenoxyphenyl)quinazolin-4-amine\|N-(4-{4-Amino-1-[1-(Tetrahydro-2h- | |
| | Pyran-4-Yl)piperidin-4-Yl]-1h-Pyrazolo[3,4-D]pyrimidin-3-Yl}-2-Methoxyphenyl)-1-Methyl-1h- | |
| | Indole-2-Carboxamide\|1H-Indole-2-carboxamide,N-(4-(1-(trans-4-(4-acetyl-1- | |
| | piperazinyl)cyclohexyl)-4-amino-1H-pyrazolo(3,4-d)pyrimidin-3-yl)-2-methoxyphenyl)-1-methyl- | |
| | \|4-[(2S)-2-(3-Chlorophenyl)-2-hydroxyethyl]amino]-3-[7-methyl-5-(4-morpholinyl)-1H- | |
| | benzimidazol-2-yl]-2(1H)-pyridinone\|3-(dimethylamino)-N-(3-(4-hydroxybenzamido)-4- | |
| | methylphenyl)benzamide\|N-(3-fluorophenyl)-6,7-dimethoxy-1,4-dihydroindeno[1,2-c]pyrazol-3- | |
| | amine\|Bisindolylmaleimide IV\|4-(Phenylsulfanyl)-7H-pyrrolo[2,3-d]pyrimidine\|Tyrphostin | |
| | A25\|Bisindolylmaleimide i\|Y-c[D-Pen-(3-1)Tyr-GSFC]KR-NH2\|4-(3-Chloro-phenoxy)-6,7- | |
| | dimethoxy-quinazoline\|Y-c[D-Pen-(3,5-dil)Tyr-GSFC]KR-NH2\|6,7-dimethoxy-N-(3,4,5- | |
| | trimethoxyphenyl)quinazolin-4-amine\|N-(4-{4-Amino-1-[4-(4-Methylpiperazin-1-Yl)-Trans- | |
| | Cyclohexyl]-1h-Pyrazolo[3,4-D]pyrimidin-3-Yl}-2-Methoxyphenyl)-1-Methyl-1h-Indole-2- | |
| | Carboxamide\|Lavendustin A\|Unii-3mae0mxi6K\|(S)-4-(2-(N-methylisoquinoline-5-sulfonamido)-3- | |
| | oxo-3-(4-phenylpiperazin-1-yl)propyl)phenyl isoquinoline-5-sulfonate\|4,5,6,7- | |
| | Tetrabromobenzotriazole\|6-o-Tolylquinazolin-2-amine\|6-(2,6-dichlorophenyl)-8-methyl-2-((3- | |
| | (methylthio)phenyl)amino)pyrido[2,3-d]pyrimidin-7(8H)-one\|2-chloro-5-(2-phenyl-5-(pyridin-4-yl)- | |
| | 1H-imidazol-4-yl)phenol\|4-(4-(3-(pyridin-2-yl)-1H-pyrazol-4-yl)pyridin-2-yl)-N-(tetrahydro-2H- | |
| | pyran-4-yl)benzamide\|bisindolylmaleimide IX\|6-(2,6-Dichlorophenyl)-2-{[3- | |
| | (hydroxymethyl)phenyl]amino}-8-methylpyrido[2,3-D]pyrimidin-7(8H)-one\|6-(2,6-Dichlorophenyl)- | |
| | 8-methyl-2-(4-morpholin-4-ylanilino)pyrido[2,3-d]pyrimidin-7-one\|Pyrido(2,3-d)pyrimidin-7(8H)- | |
| | one,6-(2,6-dichlorophenyl)-2-((4-fluorophenyl)amino)-8-methyl-\|2-[(4-Ethoxyphenyl)amino]-8- | |
| | methyl-6-(2,6-dichlorophenyl)pyrido[2,3-d]pyrimidine-7(8H)-one\|2-[4-[2- | |
| | [Ethyl(acetyl)amino]ethoxy]phenyl]amino]-8-methyl-6-(2,6-dichlorophenyl)pyrido[2,3- | |
| | d]pyrimidine-7(8H)-one\|6-(2,6-Dichlorophenyl)-2-[(4-Fluoro-3-Methylphenyl)amino]-8- | |
| | Methylpyrido[2,3-D]pyrimidin-7(8h)-One\|N3-(4-fluorophenyl)-1h-pyrazolo[3,4-d]pyrimidine-3,4- | |
| | diamine\|(3z)-N,N-Dimethyl-2-Oxo-3-(4,5,6,7-Tetrahydro-1h-Indol-2-Ylmethylidene)-2,3-Dihydro- | |
| | 1h-Indole-5-Sulfonamide\|1-tert-butyl-3-(naphthalen-1-ylmethyl)-1H-pyrazolo[3,4-d]pyrimidin-4- | |
| | amine\|2-Methoxy-12-(2-hydroxyethyl)-13,14-dihydronaphthol[2,1-a]pyrrolo[3,4-c]carbazole-5- | |
| | one\|3-({4-[(2-Methylphenyl)amino]pyrimidin-2-yl}amino)benzamide\|Indiplon\|\|(+/−)-2-(2- | |
| | Cinnamamidobenzamido)-3-phenylpropanoic acid\|CID 73755245\|Tozasertib\|Dasatinib | |
| PI4KB | N-(5-(4-Chloro-3-(N-(2-hydroxyethyl)sulfamoyl)phenyl)-4-methylthiazol-2-yl)acetamide | Inhibitor |
| KC1A | 2-[2-(4-fluoro-2-methylphenoxymethyl)-5-methoxypyridin-4-yl]-1H,4H,5H,6H,7H-pyrrolo[3,2- | Inhibitor |
| | c]pyridin-4-one\|3-[(2,4,6-Trimethoxy-phenyl)-methylene]-indolin-2-one\|CK1 Inhibitor\|N-(1-((2- | |
| | (Dimethylamino)ethyl)amino)-2-methyl-1-oxopropan-2-yl)-4-(4-(2-methyl-5-((2S,3R,4R,5S,6R)- | |
| | 3,4,5-trihydroxy-6-(methylthio)tetrahydro-2H-pyran-2-yl)benzyl)phenyl)-butanamide\|(4R)-3,7,7- | |
| | trimethyl-4-phenyl-2,3,3a,4,6,8,9,9a-octahydro-1H-pyrazolo[3,4-b]quinolin-5-one | |
| E2AK3 | 1-(5-(4-amino-7-methyl-7H-pyrrolo[2,3-d]pyrimidin-5-yl)indolin-1-yl)-2-(3- | Inhibitor |
| | (trifluoromethyl)phenyl)ethanone\|2-(2,5-Difluorophenyl)-1-[5-[(6,7-dimethoxyquinazolin-4- | |
| | yl)amino]-4-fluoro-2,3-dihydroindol-1-yl]ethanone\|Sultiame\|6-bromo-5-methyl-1H-indole-2,3- | |
| | dione\|1-[4-(4-Amino-7-methylpyrrolo[2,3-d]pyrimidin-5-yl)-3-fluorophenyl]-3-(3,5- | |
| | difluorophenyl)pyrrolidin-2-one\|2-(4-Bromo-2-methylpyrazol-3-yl)-1-[5-[(6,7-dimethoxyquinazolin- | |
| | 4-yl)amino]-2,3-dihydroindol-1-yl]ethanone\|1-[4-(4-Amino-7-methylpyrrolo[2,3-d]pyrimidin-5- | |
| | yl)phenyl]-3-methyl-3-phenylpyrrolidin-2-one\|9-Mercaptomethyl-10-oxo-azecane-2-carboxylic | |
| | acid\|1-[5-[(6,7-dimethoxyquinazolin-4-yl)amino]-2,3-dihydroindol-1-yl]-2-(2-methyl-1H-indol-3- | |
| | yl)ethanone\|1-[4-(4-Amino-7-methylpyrrolo[2,3-d]pyrimidin-5-yl)phenyl]-3-hydroxy-3-[6- | |
| | (trifluoromethyl)pyridin-2-yl]pyrrolidin-2-one\|1-[4-(4-Amino-7-methylpyrrolo[2,3-d]pyrimidin-5- | |
| | yl)phenyl]-3-(3,5-dimethylpyrazol-1-yl)pyrrolidin-2-one\|(3S,4R)-3-hydroxy-2,2-dimethyl-4-(3- | |
| | oxocyclopenten-1-yl)oxy-3,4-dihydrochromene-6-carbonitrile\|c[RGDf-(S,R)-alpha-Dfm-F]\|8-[4-(4- | |
| | Methylpiperazin-1-yl)phenoxy]-4,5-dihydro-[1,2]oxazolo[4,3-h]quinazoline-3-carboxamide\|2′- | |
| | Fluor-uridine\|2-Methoxyapomorphine\|Bifenox\|5-Heptyl-6-hydroxy-1,3-benzothiazole-4,7-dione | |
| ILK | 2-[4-(dimethylamino)phenyl]-3H-naphtho[3,2-e]benzimidazole-6,11-dione\|Ilk-IN-1\|U.S. Pat No. 8,754,233, | Inhibitor |
| | 2-(3-Amino-1H-pyrazol-4-yl)-5-fluoro-benzothiazole-6-sulfonic acid amide\|U.S. Pat No. 8,754,233,(4- | |
| | Benzothiazol-2-yl-1H-pyrazol-3-yl)-[2-(1H-imidazol-4-yl)-ethyl]-amine\|U.S. Pat No. 8,754,233, 4-(5-Fluoro-6- | |
| | methoxy-benzothiazol-2-yl)-5-(2-fluoro-phenyl)-2H-pyrazol-3-ylamine | |
| ERBB2 | Huperzine A\|8-methyl-8-azabicyclo[3.2.1]oct-3-yl 1H-indole-3-carboxylate\|Redoxal\|(1-Benzyl- | CAR-T-Cell- |
| | 1H-indol-5-yl)-quinazolin-4-yl-amine\|(1-Benzyl-1H-indazol-5-yl)-quinazolin-4-yl-amine\|3- | Therapy\| |
| | Aminobenzenesulfonamide\|N6,N6-dimethyl-N4-[1-(phenylmethyl)-5-indazolyl]pyrido[3,4- | Binder\|.\| |
| | d]pyrimidine-4,6-diamine\|CID 23712772\|Mubritinib\|2-(4-Isobutylcyclohexyl)-2-oxoethyl | Inhibitor\| |
| | benzenesulfonate\|2,2,2-trifluoro-N-[(1R,2S)-1-[1-(4-fluorophenyl)indazol-5-yl]oxy-1-(3- | Modulator\| |
| | methoxyphenyl)propan-2-yl]acetamide\|Sodium dodecyl sulfate\|Tak-285\|cis-2-(Para- | Enhancer\| |
| | fluorophenyl)cyclopropylamine\|Osi-027\|Phosphomethylphosphonic acid adenosyl | Immuno- |
| | ester\|Buspirone\|Pegunigalsidase alfa\|6-[5-[2-(Methylsulfonyl)ethyl]amino]methyl]-2-furanyl]-N- | modulator\| |
| | [3-methyl-4-([1,2,4]triazolo[1,5-a]pyridin-7-yloxy)phenyl]-4-quinazolinamine\|Fenretinide\|Cudc- | Antagonist |
| | 101\|CID 73755245\|Bedoradrine sulfate\|Diethylcarbamazine\|Palodesangren E\|(5Z)-5-[(3,5-ditert- | |
| | butyl-4-hydroxyphenyl)methylidene]-1,3-thiazolidin-4-one\|Pibaxizine\|TP- | |
| | 271\|Odevixibat\|Desidustat\|(1s,2s,3r,6r)-4-(Hydroxymethyl)-6-(Octylamino)cyclohex-4-Ene-1,2,3- | |
| | Triol\|Compstatin 40\|Cathelicidin\|17-Methoxy-5,7-dioxa-24- | |
| | azoniahexacyclo[11.11.0.02,10.04,8.014,19.020,24]tetracosa-1(24),3,8,11,13,15,17,19-octaen- | |
| | 18-ol; chloride\|Simufilam\|Zeteletinib hemiadipate\|N-tert-butylphenothiazine-10- | |
| | carboxamide\|Benzamide,4-(4-(((6-methoxy-2-(2-methoxyimidazo(2,1-b)-1,3,4-thiadiazol-6-yl)-4- | |
| | benzofuranyl)oxy)methyl)-2-thiazolyl)-N,N-dimethyl-\|Belvarafenib\|Paxilline\|Biguanide\|CID | |
| | 21226412\|Leupeptin\|2-(2-{2-[5-Amino-2-(4-fluoro-phenyl)-6-oxo-6H-pyrimidin-1-yl]-acetylamino}- | |
| | 3-phenyl-propionyl)-benzooxazole-5-carboxylic acid methyl ester\|Filorexant\|N-Hydroxy-N- | |
| | methyl-3-naphthalen-2-yl-propionamide\|4-Amino-N-(1-aza-tricyclo[3.3.1.0*3,7*]non-4-yl)-5- | |
| | chloro-2-methoxy-benzamide; hydrochloride\|4-{[3-(2-phenylethynyl)phenyl]methyl}-1H- | |
| | imidazole\|Tuxobertinib\|Hexasodium phytate\|3-[5-Chloranyl-6-[(1~{r})-1-(6-Methylpyridazin-3- | |
| | Yl)ethoxy]-1,2-Benzoxazol-3-Yl]propanoic Acid\|1-Propanesulfonamide,3,3,3-trifluoro-N-[trans-4- | |
| | [3-[(2S)-2-methyl-4-morpholinyl]-1H-pyrazolo[3,4-d]pyrimidin-6-yl]amino]cyclohexyl]-\|S-(2- | |
| | Aminophenyl)-L-cysteine S,S-dioxide\|Sapitinib\|#N/A\|2-Methoxy-N-[(2E)-3-[4-[3-methyl-4-[(6- | |
| | methyl-3-pyridinyl)oxy]phenyl]amino]-6-quinazolinyl]-2-propen-1-yl]acetamide\|4-(4- | |
| | Fluoronaphthalen-1-yl)-6-isopropylpyrimidin-2-amine\|Carbidopa\|6-[5-(3-Cyano-5-fluorophenyl)- | |
| | 1,2,4-oxadiazol-3-yl]pyridine-3-carbonitrile\|Bryamycin\|3-Phenyl-1-(6,7,8,9- | |
| | tetrahydrobenzo[g][1,3]benzodioxol-6-ylmethyl)pyrrolidine\|5H-quinolino[8,7-c][1,2]benzothiazine | |
| | 6,6-dioxide\|Poziotinib\|Phenacetin\|(S)-Morpholin-3-ylmethyl (4-((1-(3-fluorobenzyl)-1H-indazol-5- | |
| | yl)amino)-5-methylpyrrolo[2,1-f][1,2,4]triazin-6-yl)carbamate\|Canertinib\|N-(6,6-dimethyl-5-(1- | |
| | methylpiperidine-4-carbonyl)-1,4,5,6-tetrahydropyrrolo[3,4-c]pyrazol-3-yl)-3- | |
| | methylbutanamide\|Z-Phe-Ala-Diazomethylketone\|Cadazolid\|Tarloxotinib\|Varlitinib\|Lithocholic | |
| | acid\|2-(4-Hydroxy-phenyl)-1-p-tolyl-3H-inden-5-ol\|Amonafide\|3-aminopropyl- | |
| | (difluoromethyl)phosphinic Acid\|Nelipepimut-S\|5-bromo-N-quinolin-8-ylthiophene-2- | |
| | sulfonamide\|Neratinib\|Tucatinib\|2-[3,4-Dioxo-2-(pyridin-4-ylamino)cyclobuten-1-yl]amino]-2-(3- | |
| | hydroxyphenyl)acetamide\|Nortriptyline\|Merimepodib\|Lapatinib\|N-(2-(4-(2-Oxo-2,3-dihydro-1H- | |
| | benzo[d]imidazol-1-yl)piperidin-1-yl)ethyl)-2- | |
| | naphthamide\|Dacomitinib\|Masoprocol\|Afatinib\|Neratinib maleate\|Bifonazole\|EGFR/ErbB-2 | |
| | Inhibitor\|EGFR/ErbB-2/ErbB-4 Inhibitor\|2-methyl-N-[2-({3-[2-(prop-2- | |
| | enamido) acetamido]phenyl}amino)pyrimidin-5-yl]-5-[3-(trifluoromethyl)benzamido]benzamide\|6- | |
| | Chloro-N,N-diethyl-7-fluoro-4-phenyl-3-(2H-tetrazol-5-yl) quinolin-2-amine\|2- | |
| | Naphthalenecarboxamide,N-(4-(4-(2-methoxyphenyl)-1-piperazinyl)butyl)-\|3,4-Dibromo-5-[(2,3- | |
| | dibromo-4,5-dihydroxyphenyl)methyl]benzene-1,2-diol\|Methyl 3-[4-[5-(4-fluorophenyl)-2- | |
| | pyrimidin-2-ylpyrazol-3-yl]piperidin-1-yl]-2-phenylpropanoate\|5-methyl-N-[6-(2-methylpyridin-3- | |
| | yl)oxypyridin-3-yl]-6-(trifluoromethyl)-2,3-dihydroindole-1-carboxamide\|N-[4-[2-(6,7-dimethoxy- | |
| | 3,4-dihydro-1H-isoquinolin-2-yl)ethyl]phenyl]-2-[(4-methylbenzoyl)amino]benzamide\|4-[(2S)-2- | |
| | (3-Chlorophenyl)-2-hydroxyethyl]amino]-3-[7-methyl-5-(4-morpholinyl)-1H-benzimidazol-2-yl]- | |
| | 2(1H)-pyridinone\|1-(4-fluorophenyl)-2-(1-(4-hydroxyphenyl)-1H-tetrazol-5-ylthio)ethanone\|4-[(3- | |
| | Bromophenyl)amino]-6-acrylamidoquinazoline\|N-(4-m-Tolylamino-quinazolin-6-yl)-acrylamide\|4- | |
| | (3-phenoxylphenyl)-5-cyano-2H-1,2,3-triazole\|4-(4-Bromophenyl)-1H-1,2,3-triazole-5- | |
| | carbonitrile\|4-(4-chlorophenyl)-5-cyano-2H-1,2,3-triazole\|4-(4-isopropylphenyl)-5-cyano-2H- | |
| | 1,2,3-triazole\|2-(4-Methoxyphenyl)-3-phenylmaleimide\|N-(4-((3-bromophenyl)amino)quinazolin- | |
| | 6-yl)but-2-ynamide\|2-(1H-Indole-3-yl)-3-phenylmaleimide\|1H-Pyrrole-2,5-dione,3,4-bis(4- | |
| | methoxyphenyl)-\|3,4-diphenyl-1H-pyrrole-2,5-dione\|Tyrphostin 47\|4-(Bis(2-chloroethyl)amino)-N- | |
| | p-tolylbenzamide\|(3R,4R)-4-Amino-1-[4-[(3-methoxyphenyl)amino]pyrrolo[2,1-f][1,2,4]triazin-5- | |
| | yl]methyl]piperidin-3-ol | |
| PI51A | CID 49853637 | Inhibitor |
| VGFR2 | (2E)-2-{[5-(4-bromophenyl)furan-2-yl]methylidene}hydrazinecarbothioamide\|N-(3-(5-chloro-1H- | .\|Inhibitor\| |
| | pyrrolo[2,3-b]pyridine-3-carbonyl)-2,4-difluorophenyl)propane-1- | Antagonist\| |
| | sulfonamide\|Cyclopropanecarboxamide,N,N′-((1,1′-biphenyl)-4,4′-diylbis(5,2-thiazolediyl-(2S)- | Modulator\| |
| | 2,1-pyrrolidinediyl((1R)-2-oxo-1-phenyl-2,1-ethanediyl)))bis-\|Sitravatinib\|(E)-(thiophen-2- | Binder |
| | ylmethylidene)amino benzoate\|lodipamide\|H-DL-Tyr(2,6-diMe)-DL-Tic-DL-Lys(Cbz)-NHPh\|(E)-1- | |
| | (4-((6-amino-5-((methoxyimino)methyl)pyrimidin-4-yl)oxy)-2-chlorophenyl)-3-ethylurea\|alpha- | |
| | (((2-Bromoethyl)amino)methyl)-2-nitro-1H-imidazole-1-ethanol monohydrobromide\|1-(4-((6,7- | |
| | Dimethoxyquinolin-4-yl)oxy)-2-methoxyphenyl)-3-(1-(thiazol-2-yl)ethyl)urea\|VEGF Receptor 2 | |
| | Kinase Inhibitor \|\|VEGF Receptor 2 Kinase Inhibitor \|\|\|Pyrazolo[1,5-a]pyrimidine,6-(4- | |
| | methoxyphenyl)-3-(3-thienyl)-IN-(4-Chlorophenyl)-2-[(pyridin-4-ylmethyl)amino]benzamide\|1- | |
| | cyclopentyl-3-(1H-pyrrolo[2,3-b]pyridin-5-yl)-1H-pyrazolo[3,4-d]pyrimidin-4-amine\|4-(4-(3-(1H- | |
| | Pyrazol-4-yl)pyrazolo[1,5-a]pyrimidin-6-yl)phenyl)morpholine\|3-[(4-chlorophenyl)-(1H-imidazol-2- | |
| | yl)methylidene]-5-[(1-ethylpiperidin-4-yl)amino]-1H-indol-2-one\|1-(4-((4-Ethylpiperazin-1- | |
| | yl)methyl)-3-(trifluoromethyl)phenyl)-3-(4-((6-(methylamino)pyrimidin-4- | |
| | yl)oxy)phenyl)urea\|Nintedanib Impurity A; BIBF 1202\|N-[3-[5-Iodo-4-[3-[(2- | |
| | thienylcarbonyl)amino]propyl]amino]-2-pyrimidinyl]amino]phenyl]-1-pyrrolidinecarboxamide\|N-[3- | |
| | [5-Bromo-4-[2-(1H-imidazol-5-YL)ethyl]amino]-2-pyrimidinyl]amino]phenyl]-1- | |
| | pyrrolidinecarboxamide\|Pyrazolo[1,5-a]pyrimidine 1a\|Benzimidazole 2\|N-(4- | |
| | phenoxyphenyl)quinazolin-4-amine\|2-Quinolinone deriv. 3\|Benzoannulenone 1\|#N/A\|N-[2,4- | |
| | dichlorobenzoyl]phenylsulfonamide\|4-Chloro-N-(2-chlorobenzoyl)benzenesulfonamide\|3-chloro- | |
| | N-(4-chlorophenyl)sulfonylbenzamide\|4-Chloro-N-[(4-chlorophenyl)sulfonyl]benzamide\|N,5- | |
| | diphenyl-1,3-oxazol-2-amine\|2-[(5-Phenyl-1,3-oxazol-2-yl)amino]benzonitrile\|N-(2- | |
| | methoxyphenyl)-5-phenyl-1,3-oxazol-2-amine\|3-[(5-Phenyl-1,3-oxazol-2-yl)amino]benzonitrile\|N- | |
| | (3-phenoxyphenyl)-5-phenyl-1,3-oxazol-2-amine\|3-(5-Phenyl-oxazol-2- | |
| | ylamino)phenylmethanol\|N-(4-chlorobenzenesulfonyl)-2-methylbenzamide\|3-bromo-N-(4- | |
| | chlorophenyl)sulfonylbenzamide\|N-(4-chlorophenyl)sulfonyl-4-nitrobenzamide\|N-(3- | |
| | methylphenyl)-5-phenyl-1,3-oxazol-2-amine\|4-[(5-Phenyl-1,3-oxazol-2- | |
| | yl)amino]benzenesulfonamide\|3-methyl-1H-thieno[2,3-c]pyrazole-5-carboxamide\|N-(4-chloro-2- | |
| | fluorophenyl)-6,7-dimethoxyquinazolin-4-amine\|4-(4-(4- | |
| | (Chlorodifluoromethoxy)phenylamino)phthalazin-1-yl)benzamide\|4-(4-(4-Tert- | |
| | butylphenylamino)phthalazin-1-yl)benzamide\|4-(4-m-Tolylamino-phthalazin-1-yl)-benzamide\|4- | |
| | (4-p-Tolylamino-phthalazin-1-yl)-benzamide\|N,N-dimethyl-5-[14-oxo-7-(propan-2-yloxymethyl)- | |
| | 3,13-diazahexacyclo[14.7.0.02,10.04,9.011,15.017,22]tricosa- | |
| | 1(16),2(10),4(9),5,7,11(15),17,19,21-nonaen-3-yl]pentanamide\|4-[(2S)-2-(3-Chlorophenyl)-2- | |
| | hydroxyethyl]amino]-3-[7-methyl-5-(4-morpholinyl)-1H-benzimidazol-2-yl]-2(1H)-pyridinone\|K- | |
| | 252a analogue\|5-[(Z)-(5-chloro-2-oxo-1,2-dihydro-3H-indol-3-ylidene)methyl]-N-[2- | |
| | (diethylamino)ethyl]-2,4-dimethyl-1H-pyrrole-3-carboxamide\|Pyrazolo[1,5-a]pyrimidine 4e\|2- | |
| | (Pyridin-4-Ylmethylamino)-~{n}-[3-(Trifluoromethyl)phenyl]benzamide\|3-[5-(3-Thienyl)-3- | |
| | pyridinyl]-1H-indole\|Unii-3mae0mxi6K\|3-(4-Aminophenyl)thieno[3,2-c]pyridin-4-amine\|4-Phenyl- | |
| | 2-(phenylamino)pyrimidine-5-carbonitrile\|2-(p-Toluidino)-4-phenylpyrimidine-5-carbonitrile\|3-((3- | |
| | Bromothiophen-2-yl)methylene)indolin-2-one\|8-methyl-4H,7H-indolo[6,5,4-cd]indol-5-one\|4-[3- | |
| | Hydroxyanilino]-6,7-dimethoxyquinazoline\|3-Phenyl-1,4-dihydroindeno[1,2-c]pyrazole\|2-(4- | |
| | Methoxyphenyl)-3-phenylmaleimide\|2-(Pyrimidin-4-ylamino)thiazole-5-carbonitrile\|6-o- | |
| | Tolylquinazolin-2-amine\|4-(4-aminophenyl)-1H-indazol-3-amine\|(4-Chlorophenyl)-(4-isoquinolin- | |
| | 5-yl-phthalazin-1-yl)amine\|4-(isoquinolin-5-yl)-N-m-tolylphthalazin-1-amine\|4-(isoquinolin-5-yl)-N- | |
| | o-tolylphthalazin-1-amine\|3-(1H-Benzimidazol-2-YL)-1H-indazole\|7-(4-fluoroanilino)-1-[(1R,3R)- | |
| | 3-hydroxycyclopentyl]-3-(4-methoxyphenyl)-4H-pyrimido[4,5-d]pyrimidin-2-one\|1H-Pyrrole-2,5- | |
| | dione,3,4-bis(4-methoxyphenyl)-\|3,4-diphenyl-1H-pyrrole-2,5-dione\|5-isopropyl-6-(5-methyl- | |
| | 1,3,4-oxadiazol-2-yl)-N-(2-methyl-1H-pyrrolo[2,3-b]pyridin-5-yl)pyrrolo[2,1-f][1,2,4]triazin-4- | |
| | amine\|2-Methoxy-12-(2-hydroxyethyl)-13,14-dihydronaphthol[2,1-a]pyrrolo[3,4-c]carbazole-5- | |
| | one\|3-(5,6,7,13-Tetrahydro-9-((1-methylethoxy)methyl)-5-oxo-12h-indeno(2,1-a)pyrrolo(3,4- | |
| | c)carbazol-12-yl)propanol\|Erythribyssin L\|5-{[(3Z)-5-fluoro-2-oxo-2,3-dihydro-1H-indol-3- | |
| | ylidene]methyl}-N-[(2S)-2-hydroxy-3-(morpholin-4-yl)propyl]-2,4-dimethyl-3H-pyrrole-3- | |
| | carboxamide\|Motesanib\|Duloxetine\|Tyrosine kinase-IN-1\|VEGF receptor tyrosine kinase inhibitor | |
| | III\|N,2-Dimethyl-6-{[7-(2-Morpholin-4-Ylethoxy)quinolin-4-Yl]oxy}-1-Benzofuran-3- | |
| | Carboxamide\|(2R)-2-amino-3-[1-[3-[2-[4-[1,3-bis(2-methoxyethylcarbamoyloxy)propan-2- | |
| | yloxy]butanoylamino]ethylamino]-3-oxopropyl]-2,5-dioxopyrrolidin-3-yl]sulfanylpropanoic | |
| | acid\|Golvatinib\|Osi-930\|Tak-593\|4-Methyl-5-(2-(4-morpholinophenylamino)pyrimidin-4-yl)thiazol- | |
| | 2-amine\|Altiratinib\|Cep-11981\|8-fluoro-12-(4-methylpiperazin-1-yl)-6H-[1]benzothieno[2,3- | |
| | b][1,5]benzodiazepine\|(2R)-4-(1H-indol-3-yl)-2-[2-(4-piperidin-4- | |
| | ylbutanoylamino)acetyl]amino]butanoic acid\|Zanoterone\|MK-2461\|Fluconazole\|Delphinidin\|3-((4- | |
| | Bromo-2,6-difluorobenzyl)oxy)-5-(3-(4-(pyrrolidin-1-yl)butyl)ureido)isothiazole-4-carboxamide\|1- | |
| | Benzyl-3-hydroxy-4-[(1-phenylethylamino)methyl]pyridin-2-one\|(3R,4R)-4-Amino-1-[4-[(3- | |
| | methoxyphenyl) amino]pyrrolo[2,1-f][1,2,4]triazin-5-yl]methyl]piperidin-3-ol\|Vatalanib\|1-methyl-5- | |
| | [2-[5-(trifluoromethyl)-1H-imidazol-2-yl]pyridin-4-yl]oxy-N-[4-(trifluoromethyl)phenyl]benzimidazol- | |
| | 2-amine\|Foretinib\|Telatinib\|Famitinib\|Sulfatinib\|Cediranib\|Brivanib\|Lucitanib\|Fruquintinib\|Orantinib\| | |
| | Neratinib\|Tivozanib\|Lenvatinib\|Apatinib Mesylate\|Semaxanib\|Lanatoside | |
| | A\|Axitinib\|Sunitinib\|Sorafenib\|Regorafenib\|Pazopanib | |
| | hydrochloride\|Vandetanib\|Cabozantinib\|6,7-Dimethyl-2-phenylquinoxaline\|1H-3-Benzazepine,6- | |
| | chloro-2,3,4,5-tetrahydro-3-methyl-9-((3-methyl-2-butenyl)oxy)-\|N-benzyl-4-(5-formyl-6- | |
| | hydroxynaphthalen-2-yl)-N-methylbenzamide\|Tupichinol C\|2-(Tert-butylamino)-1-(3,4- | |
| | dichlorophenyl)pentan-1-one\|Bardoxolone methyl\|Almorexant\|BzATP\|Cyclopentylcytosine | |
| | triphosphate | |
| FAK1 | N-(4-Tert-Butylbenzyl)-1,5-Dimethyl-1,5-Dihydropyrazolo[4,3-C][2,1]benzothiazin-8-Amine 4,4- | Inhibitor\| |
| | Dioxide\|4-[[(2S)-2-(3-Chlorophenyl)-2-hydroxyethyl]amino]-3-[7-methyl-5-(4-morpholinyl)-1H- | Modulator |
| | benzimidazol-2-yl]-2(1H)-pyridinone\|3,4-Dihydro-6-[4-[3-(methylsulfonyl)phenyl]methyl]amino]- | |
| | 5-(trifluoromethyl)-2-pyrimidinyl]amino]-2(1H)-quinolinone\|Gsk2256098\|Benzamide,2-[2-[2- | |
| | methoxy-4-(4-morpholinyl)phenyl]amino]-5-(trifluoromethyl)-4-pyridinyl]amino]-N-methyl-\|PF- | |
| | 00562271 free base\|3-Butyl-1-(naphthalen-1-yl)piperidin-4-amine\|Cep-37440\|lumateperone | |
| | Tosylate\|Defactinib\|Tyr-Pro-Phe-Hfe-NH2 | |
| PDK1 | Dichloroacetate\|9-(6-fluoropyridin-3-yl)-2-(2-methoxypyridin-4-yl)-7,8-dihydro-4H- | Inhibitor |
| | [1,4]diazepino[7,1-a]isoquinolin-5-one\|2-[(2,6-Dichlorobenzoyl)amino]pentanedioic | |
| | acid\|Caricotamide\|peptide 4 [PMID: 18294843]\|Levothyroxine\|(S)-(7-chloroimidazo[1,5-a]pyridin- | |
| | 5-yl)-cyclohexylmethanol\|Phlorizin\|H-Tyr-D-Ala-Gly-Phe-Pro-Leu-Trp-O-Bzl\|Potassium | |
| | Iodide\|(S)-3-Amino-4,6-dimethyl-N-(1-phenylethyl)thieno[2,3-b]pyridine-2-carboxamide 2,2,2- | |
| | trifluoroacetate\|N-(3-(4-(3-(Diisobutylamino)propyl)piperazin-1-yl)propyl)-1H-benzo(d)imidazol-2- | |
| | amine disulphate salt\|Nitroaspirin\|1,3-Diallyl-3,7-dihydro-purine-2,6-dione\|2-(4-hydroxypent-1-yl)- | |
| | N6-methoxyadenosine\|[(2R)-3-hexadecoxy-2-(methylcarbamoyloxy)propyl]2- | |
| | (trimethylazaniumyl)ethyl phosphate\|Pregnan-20-one,3-hydroxy-3-(trifluoromethyl)-, | |
| | (3alpha,5alpha)-\|2,6-Naphthalenedicarboxamide,n2,n6-dicyclohexyl-\|4,5-Bis[4- | |
| | methylanilino]phthalimide\|2-(2,4-Dichlorophenoxy)-5-(4-fluorophenyl)phenol\|(5Z)-5-[[4-[(1- | |
| | pyridin-2-ylpyrrolidin-2-yl)methoxy]phenyl]methylidene]-1,3-thiazolidine-2,4-dione\|4- | |
| | Phenylimidazole\|1-Aminopentylphosphonic acid monophenyl ester\|d[Val4]AVP\|5-[2,3- | |
| | bis(fluoranyl)phenyl]-~{N}-[1-(2-methoxyethyl)piperidin-4-yl]methyl]-1~{H}-indazole-3- | |
| | carboxamide\|6-[(2-amino-1,2-dihydroimidazol-3-yl)methyl]-5-chloro-1H-pyrimidine-2,4- | |
| | dione\|Mirvetuximab soravtansine\|(R/S)-2-((4-benzylphenoxy)methyl)piperidine\|7-(3-Fluoro-5- | |
| | methylphenyl)-1,6-naphthyridine\|1-(3-(4-(2-Fluoroethyl)phenoxy)propyl)piperidine\|1-Pyrazin-2-yl- | |
| | 3-(6-trifluoromethyl-quinolin-4-yl)-urea\|6-Fluoro-N-(2-morpholinoethyl)nicotinamide\|1-[3-[4-[3,5- | |
| | Bis(trifluoromethyl)phenyl]piperidine-1-carbonyl]-1,4,5,7-tetrahydropyrazolo[3,4-c]pyridin-6- | |
| | yl]ethanone\|6-methyl-2,3,4,5-tetrahydro-1H-azepino[4,5-b]indole\|7-[(2S,3R)-3-amino-2- | |
| | methylazetidin-1-yl]-8-chloro-1-cyclopropyl-6-fluoro-4-oxoquinoline-3-carboxylic acid\|(5-Methyl- | |
| | 4,5-dihydro-1H-pyrazol-1-yl)(pyridin-3-yl)methanone\|1-(2-Hydroxy-3-nitro-phenyl)-3-phenyl- | |
| | urea\|3-Nitro-beta-carboline\|Sulopenem etzadroxil\|3-Methoxy-5-(7-methylpyrido[2,3-d]pyrimidin- | |
| | 2-yl)benzonitrile\|Benzamide,3-((1E)-2-(6-(cyclopropylamino)-9H-purin-9-yl)ethenyl)-4-methyl-N- | |
| | (4-(trifluoromethyl)-2-pyridinyl)-\|CID 9799221\|1-(5-Carbamoylpyridin-2-yl)piperidin-4-yl 4- | |
| | isopropylpiperazine-1-carboxylate\|N-(6-(2-Aminophenylamino)-6-oxohexyl)-4- | |
| | methylbenzamide\|Aspirin\|[(4R,4aR,7S,7aR,12bS)-3-methyl-9-propanoyloxy-1,2,3,4,4a,7,7a,13- | |
| | octahydro-4,12-methanobenzofuro[3,2-e]isoquinolin-3-ium-7-yl] sulfate\|GW 583340 | |
| | dihydrochloride\|Mitoflaxone\|Thioctic acid\|Paeoniflorin\|#N/A\|N-(2-Chloro-6-propoxypyridin-4-yl)-2- | |
| | (2-hydroxyethyl)-2-(4-isopropyl-1,3-dimethyl-1H-pyrazolo(3,4-b)pyridin-6- | |
| | yl)hydrazinecarboxamide\|Kukoamine A\|1-Tritylpyrrolidine\|4-(N-(4-cyclohexylbenzyl)-2-(2,3,4,5,6- | |
| | pentafluoro-N-methylphenylsulfonamido)acetamido)-2-hydroxybenzoic acid\|[(3S)-8-(5-chloro-6- | |
| | fluoro-1H-benzimidazol-2-yl)-4-(5-methoxypyridin-2-yl)-2,3-dihydro-1,4-benzoxazin-3- | |
| | yl]methanol\|4-Methoxy-2-phenyl-2H-pyrazolo[4,3-c]quinoline\|Cephaloglycin\|[(1S,2S,3R,4S)-4-(5- | |
| | amino-4-carbamoylimidazol-1-yl)-2,3-dihydroxycyclopentyl]methyl dihydrogen phosphate\|N-[N′- | |
| | Benzyloxycarbonyl-phenylalaninyl]-3-amino-5-phenyl-pentane-1-sulfonic acid phenyl | |
| | ester\|Endiandrin A\|Tonapofylline\|5-[(Imidazol-1-yl)methyl]-7,8-dihydroquinoline\|1-Methyl-5-(2- | |
| | diethylaminoethylamino)-8-methoxy-6H-imidazo[4,5,1-de]acridin-6-one\|Fggftgarksarkkanq\|(Z)-7- | |
| | [(1R,5S,6R,7R)-7-[(3S)-3-hydroxyoctyl]-3-oxo-2,4-dioxabicyclo[3.2.1]octan-6-yl]hept-5-enoic | |
| | acid\|(1E,4E)-1,5-bis(4-hydroxy-3-methoxyphenyl)penta-1,4-dien-3-one\|H-DL-Tyr(2,6-diMe)-DL- | |
| | Tic-DL-Lys-NHPh\|4-butyl-N-(quinolin-3-yl)benzamide\|Vortioxetine\|Jtc-801\|Octyl thiophosphatidic | |
| | acid\|[3H]-(2R)-3-[(2R,6S)-2,6-dimethylpiperidin-1-yl]-2-hydroxy-S-(naphthalen-1-yl)propane-1- | |
| | sulfonamido\|Lutiram\|beta-Lactose\|(E,E)-8-[4-(3-Fluorophenyl)butadien-1-yl]caffeine\|5- | |
| | [(1S,2R,4R)-7-azabicyclo[2.2.1]heptan-2-yl]-1,2,4-oxadiazol-3-amine\|1-(6-aminopyridin-3-yl)-2- | |
| | [(1S,2R,4R)-2-bicyclo[2.2.1]heptanyl]-3-cyanoguanidine;hydrochloride\|beta- | |
| | Funaltrexamine\|Sulfamethoxazole and trimethoprim\|2-[2-Ethoxy-4-(4-pyrrolidin-1-ylpiperidine-1- | |
| | carbonyl)anilino]-5,11-dimethylpyrido[4,3-b][1,4]benzodiazepin-6-one\|4″-(Pyridin-4- | |
| | ylmethyl)biphenyl-3-ol\|1,3-Dicyclohexylurea\|Heptaplatin Sunpla\|2-(4-Phenoxy-phenyl)-1H- | |
| | benzoimidazol-5-ylamine\|Nintedanib Impurity A;BIBF 1202\|Cyclo[Ac-Cys-Ile-Tyr-Lys-Tyr- | |
| | Tyr]\|Venlafaxine\|Urea,(2,3-dihydro-2-oxo-3-(1-(1H-pyrrol-2-yl)ethylidene)-1H-indol-5-yl)-\|N-(cis- | |
| | 9-cis-12-octadecadienyl)sulfamide\|6-[3-(2,2-difluoro-12-thiophen-2-yl-3-aza-1-azonia-2- | |
| | boranuidatricyclo[7.3.0.03,7]dodeca-1(12),4,6,8,10-pentaen-4-yl)propanoylamino]-N-[4-[4-[2- | |
| | oxo-2-(6-oxo-5H-pyrido[2,3-b][1,4]benzodiazepin-11-yl)ethyl]piperazin-1-yl]butyl]hexanamide\|4- | |
| | [(3-Chloro-4-{[(2r)-3,3,3-Trifluoro-2-Hydroxy-2-Methylpropanoyl]amino}phenyl)sulfonyl]-N,N- | |
| | Dimethylbenzamide\|N-Methyl-N-(Methylbenzyl)formamide\|Urotensin-related peptide\|6,7- | |
| | Dimethoxy-3-((E)-styryl)-quinoline\|1-[(4R)-7-chloro-2,2-bis(fluoromethyl)-3,4-dihydrochromen-4- | |
| | yl]-3-[(2R)-2-hydroxy-2,3-dihydro-1H-inden-4-yl]urea\|Cetrimide\|Calcium | |
| TEC | Blk-IN-1\|2-methyl-N-[2-({3-[2-(prop-2-enamido)acetamido]phenyl}amino)pyrimidin-5-yl]-5-[3- | Inhibitor |
| | (trifluoromethyl)benzamido]benzamide\|(5-(3-methoxyphenyl)-1-pentyl-1H-pyrrol-3- | |
| | yl)(naphthalen-1-yl)methanone | |
| ULK3 | (1S,2S,3R,4R)-3-[5-Chloro-2-(1-ethyl-6-methoxy-2-oxo-2,3,4,5-tetrahydro-1H-benzo[b]azepin-7- | Inhibitor |
| | ylamino)-pyrimidin-4-ylamino]-bicyclo[2.2.1]hept-5-ene-2-carboxylic acid amide | |
| EGFR | Bifeprunox\|Oteseconazole\|Mobocertinib\|Pyrotinib\|5-methyl-N-[6-(2-methylpyridin-3-yl)oxypyridin- | Inhibitor\|.\| |
| | 3-yl]-6-(trifluoromethyl)-2,3-dihydroindole-1-carboxamide\|N-(3-(2-((2,3-Difluoro-4-(4-(2- | Antagonist\| |
| | hydroxyethyl)piperazin-1-yl)phenyl)amino)quinazolin-8-yl)phenyl)acrylamide\|Abivertinib\|8- | Modulator\| |
| | (biphenyl-4-yl)-N-hydroxy-8-oxooctanamide\|Saviprazole\|6-[(1-acetylpiperidin-4-yl)amino]-N- | CAR-T-Cell- |
| | [(2S)-2-hydroxy-3-(1,2,3,4-tetrahydroisoquinolin-2-yl)propyl]pyrimidine-4-carboxamide\|5′-{[(3s)-3- | Therapy\| |
| | Amino-3-Carboxypropyl][3-(Dimethylamino)propyl]amino}-5′- | Activator\| |
| | Deoxyadenosine\|Rusfertide\|Osimertinib\|4-(6,7-Dimethoxy-quinolin-3-yl)-benzoic acid\|N-methyl- | Suppressor |
| | 2-(3-methyl-2-oxo-6-phenylbenzimidazol-1-yl)-N-phenylacetamide\|2-[1-(2-Methylphenyl)pyrrol-3- | |
| | yl]ethanamine\|2-phenyl-5-[5-(trifluoromethyl)-1,2,4-oxadiazol-3-yl]-3H-isoindol-1-one\|Ethyl 3-(3- | |
| | bromophenyl)-5,7-dihydroxy-4-oxo-chromene-2-carboxylate\|(3R)-1,2,3,9-tetrahydropyrrolo[2,1- | |
| | b]quinazolin-3-ol\|3-(8-Iodo-5-oxo-3,4,5,6-tetrahydro-1,6-benzodiazocin-2- | |
| | yl)benzonitrile\|Indiplon\|Rienso; Hydroxy(oxo)iron,iron; C 7228; Code 7228;Cytogen;Ferrheme\|6- | |
| | (3,4-Dichlorophenyl)-1-[1-(methyloxy)-3-buten-1-YL]-3-azabicyclo[4.1.0]heptane (diastereomeric | |
| | mix)\|[(4R,4aR,7S,7aR,12bS)-9-methoxy-3-methyl-2,4,4a,7,7a,13-hexahydro-1H-4,12- | |
| | methanobenzofuro[3,2-e]isoquinolin-7-yl]hydrogen sulfate\|Silibinin B\|Epitinib\|4-((2H-Tetrazol-5- | |
| | yl)methyl)piperidine-2-carboxylic acid\|1-(2-(dimethylamino)-4-(trifluoromethyl)benzyl)-3-(3,4- | |
| | dihydro-3-oxo-2H-benzo[b][1,4]oxazin-8-yl)urea\|1-(1,3,4-Oxadiazol-2-yl)octadec-9-en-1-one\|N- | |
| | Isopropyl-N′-hydroxyguanidine\|c[RGD-(S)-alpha-TfmfV]\|Guanfacine hydrochloride\|PO3 2-Nle- | |
| | Trp-O-3K\|N-(3-((5-chloro-2-((4-(4-methylpiperazin-1-yl)phenyl)amino)pyrimidin-4- | |
| | yl)oxy)phenyl)acrylamide\|2-Benzyl-4-piperazin-1-yl-1H-benzimidazole\|N-(3-Ethynylphenyl)-7- | |
| | methoxy-6-(3-(4-morpholinyl)propoxy)-4-quinazolinamine\|Theliatinib\|2-Bromo-4-[(6,7- | |
| | dimethoxyquinazolin-4-yl)amino]phenol\|EGFR Inhibitor\|EGFR/ErbB-2 Inhibitor\|EGFR/ErbB- | |
| | 2/ErbB-4 Inhibitor\|1-cyclopentyl-3-(1H-pyrrolo[2,3-b]pyridin-5-yl)-1H-pyrazolo[3,4-d]pyrimidin-4- | |
| | amine\|4-[(3-Bromophenyl)amino]-6,7-diethoxyquinazoline\|Tyrphostin AG 112\|Tyrphostin A1\|2- | |
| | (4-(Benzenesulfonyl)phenyl)-1-aminoethane\|Bpiq-i\|2-methyl-N-[2-({3-[2-(prop-2- | |
| | enamido)acetamido]phenyl}amino)pyrimidin-5-yl]-5-[3- | |
| | (trifluoromethyl)benzamido]benzamide\|4,5-Bis[4-methylanilino]phthalimide\|7-Methyl-7H- | |
| | pyrrolo[3,2-f]quinazoline-1,3-diamine\|Clomocycline\|Perfosfamide\|N-(3-chlorophenyl)-6,7- | |
| | dimethoxyquinazolin-4-amine\|N4-(3-Bromophenyl)quinazoline-4,6-diamine\|N4-(3- | |
| | chlorophenyl)quinazoline-4,6-diamine\|N4-(3-bromophenyl)-N6-methylpyrido[3,4-d]pyrimidine- | |
| | 4,6-diamine\|N-(3-bromophenyl)benzo[g]quinazolin-4-amine\|4-(3-Bromo-phenoxy)-6,7- | |
| | dimethoxy-quinazoline\|4-(3-Bromo-phenoxy)-6,7-dimethoxy-quinoline\|N-(3- | |
| | bromophenyl)quinazolin-4-amine\|4-N-(3-bromophenyl)quinazoline-4,6,7-triamine\|4-N- | |
| | benzylpyrido[4,3-d]pyrimidine-4,7-diamine\|7-Amino-4-[(3-bromophenyl)amino]quinazoline\|N-(4- | |
| | m-Tolylamino-quinazolin-7-yl)-acrylamide\|#N/A\|N-(3-bromophenyl)-5-nitroquinazolin-4- | |
| | amine\|(E)-2-benzoyl-3-(3,4-dihydroxyphenyl)prop-2-enenitrile\|Tyrphostin A46\|Tyrphostin AG | |
| | 538\|Benzyl-quinazolin-4-yl-amine\|4-[(3-Bromophenyl) amino]-6-acrylamidoquinazoline\|N-(4-m- | |
| | Tolylamino-quinazolin-6-yl)-acrylamide\|(4,5- | |
| | Dihydroxyindanylidene)malononitrile\|Propanedinitrile,(2,3-dihydro-5,6-dihydroxy-1H-inden-1- | |
| | ylidene)-\|3-Pyridin-4-yl-quinoline-6,7-diol\|Lavendustin A\|6,7-Dimethoxy-4-(2- | |
| | phenylethynyl)quinazoline\|6,7-Diethoxy-4-(5-phenylpent-1-enyl)quinazoline\|6,7-Diethoxy-4-(4- | |
| | phenylbut-1-enyl)quinazoline\|6,7-Diethoxy-4-styrylquinazoline\|4-Biphenyl-2-ylethynyl-6,7- | |
| | dimethoxy-quinazoline\|6,7-Dimethoxy-4-(3-phenoxyprop-1-ynyl)quinazoline\|6,7-Dimethoxy-4-(4- | |
| | phenylbut-1-ynyl)quinazoline\|3-(1H-imidazol-5-ylmethylene)-1,3-dihydro-2H-indol-2-one\|2-(4- | |
| | Methoxyphenyl)-3-phenylmaleimide\|N-(4-((3-bromophenyl)amino)quinazolin-6-yl)but-2- | |
| | ynamide\|N-(3-bromophenyl)-2-hydroxy-4-(prop-2-enoylamino)benzamide\|N-(3-bromophenyl)-2- | |
| | hydroxy-5-(prop-2-enoylamino)benzamide\|6-Amino-4-[(3-methylphenyl)amino]quinazoline\|2-(1H- | |
| | Indole-3-yl)-3-phenylmaleimide\|7-(4-fluoroanilino)-1-[(1R,3R)-3-hydroxycyclopentyl]-3-(4- | |
| | methoxyphenyl)-4H-pyrimido[4,5-d]pyrimidin-2-one\|1H-Pyrrole-2,5-dione,3,4-bis(4- | |
| | methoxyphenyl)-\|3,4-diphenyl-1H-pyrrole-2,5-dione\|(E)-5-(4-Hydroxybenzylidene)-1- | |
| | phenethylhydantoin\|6-(2,6-Dichlorophenyl)-2-{[3-(hydroxymethyl)phenyl]amino}-8- | |
| | methylpyrido[2,3-D]pyrimidin-7(8H)-one\|Pyrido(2,3-d)pyrimidin-7(8H)-one,6-(2,6- | |
| | dichlorophenyl)-2-((4-fluorophenyl)amino)-8-methyl-\|6-(2,6-Dichlorophenyl)-2-[(4-Fluoro-3- | |
| | Methylphenyl)amino]-8-Methylpyrido[2,3-D]pyrimidin-7(8h)-One\|Cochliobolic acid\|10-Hydroxy- | |
| | 18-methoxybetaenone\|Benzyloxycarbonylalanine chloromethyl ketone\|4-(4-(2- | |
| | Nitrovinyl)phenoxysulfonyl)benzoic acid\|4-[(E)-2-Nitroethenyl]phenol\|Tyrphostin A23\|Tyrphostin | |
| | 47\|2-Methoxy-4-(2-nitrovinyl)phenol\|4-(2-Nitroprop-1-enyl)benzene-1,2-diol\|4-[(E)-2- | |
| | nitroethenyl]benzene-1,2-diol\|6-chloro-N-(3-chlorophenyl)quinazolin-4-amine\|6,7-dimethoxy-N- | |
| | (3-methylphenyl)quinazolin-4-amine\|3-((2,6-dichlorophenoxy)methyl)-5-((4-(4- | |
| | (trifluoromethyl)phenyl)-4H-1,2,4-triazol-3-ylthio)methyl)-1,2,4-oxadiazole\|4-(4- | |
| | phenylmethoxyphenyl)-3-[2-[3-(trifluoromethyl)phenyl]sulfonylethylsulfanyl]-1H-1,2,4-triazol-5- | |
| | one\|4-(5-Bromoindole-3-yl)-6,7-dimethoxyquinazoline\|N-(4-Phenylamino-Quinazolin-6-Yl)- | |
| | Acrylamide\|3-(3-Chloro-phenyl)-5,7-dihydroxy-chromen-4-one\|3- | |
| | Aminobenzenesulfonamide\|Nepidermin\|4,5-Dianilinophthalimide\|Gingerol\|3-(6- | |
| | Methoxynaphthalen-2-yl)pyridin-4-amine\|(R)-N-[(1S,2R)-2-Hydroxyindan-1-yl]-3-[(2S,3S)-3-(3- | |
| | methylphenoxyacetyl)amino-2-hydroxy-4-phenylbutanoyl]-5,5-dimethyl-1,3-thiazolidine-4- | |
| | carboxamide\|1-(4-chlorophenyl)-2-(2,4-dichlorophenyl)-5-methylsulfonyl-N-(piperidin-1-yl)-1H- | |
| | imidazole-4-carboxamide\|((4-Phenyl-1-(4-(2-quinolinylmethoxy)phenyl)butyl)thio)acetic | |
| | acid\|Mubritinib\|N-(3-bromophenyl)-6,7-dimethoxyquinazolin-4-amine\|2H-Pyran-2-one,6-((1E)- | |
| | 4,4-bis(4-fluorophenyl)-3-(1-methyl-1H-tetrazol-5-yl)-1,3-butadien-1-yl)tetrahydro-4-hydroxy-, | |
| | (4R,6S)-rel-\|Pki-166\|Falnidamol\|4-(4′-Hydroxyphenyl)diazenylbenzenesulfonamide\|Biapenem\|N- | |
| | [(2R)-1-[4-[3-[4-(cyclopropanecarbonyl)phenoxy]propyl]piperazin-1-yl]-1-oxopropan-2-yl]furan-2- | |
| | carboxamide\|[(2-Methoxyphenyl)-sulfanylmethyl]phosphonic acid\|Allitinib\|Fenretinide\|1-deoxy-1- | |
| | (methylazaniumyl)-D-glucito[2,2′,2″,2″-(1,4,7,10-tetraazacyclododecane-1,4,7,10-tetrayl- | |
| | kappa(4)N(1),N(4),N(7),N(10)) tetraacetato(4-)]gadolinate(1-)\|Cudc-101\|CID 9865731\|CID | |
| | 73755245\|4-(aminomethyl)-N-(3-fluoropyridin-4-yl)-3-[3-[4-(2-oxooxolan-3- | |
| | yl)sulfanylbenzoyl]amino]phenyl]benzamide\|Piperazine,1-benzoyl-4-[2-(4,7-dimethoxy-1H- | |
| | pyrrolo[2,3-c]pyridin-3-yl)-1,2-dioxoethyl]-& Ac-WQEWEQKITA LLEQAQIQQE KNEYELQKLD | |
| | KWASLWEWF-NH2\|N-hydroxy-7-[pyrazin-2-yl(pyrimidin-5-yl)amino]heptanamide\|4-[3-(1- | |
| | Benzofuran-5-yloxy)phenyl]-1-phosphonatobutane-1-sulfonate\|1-(4-chlorophenyl)-4-(piperidin-1- | |
| | yl)-1H-imidazol-2(5H)-one\|(8-Methyl-8-azabicyclo[3.2.1]octan-3-yl) 2-(4- | |
| | chlorophenoxy)butanoate\|6-[6-[4-[2-[2-(1-Adamantyl)acetyl]amino]ethyl]piperazin-1-yl]pyridin-3- | |
| | yl]-N-[(4,6-dimethyl-2-oxo-1H-pyridin-3-yl)methyl]-1-propan-2-ylindazole-4- | |
| | carboxamide\|Simufilam\|2-morpholino-4H-benzo[g]chromen-4-one\|Lazertinib\|3,5- | |
| | Dithiocyanatopyridine-2,6-diamine\|Paxilline\|1H-Indole-2-carboxylic acid,4,6-dichloro-3-((2-oxo- | |
| | 1-phenyl-3-pyrrolidinylidene)methyl)-,(E)-\|Sophoraflavanone B\|Fasidotrilat[1,1′-Biphenyl]-4- | |
| | methanamine,N-(2,3-dihydro-1H-inden-2-yl)-3,5-difluoro-3′-(1H-1,2,4-triazol-5-yl)-\|4-chloro-3- | |
| | [(3-chloropyridin-2-yl)-(2-hydroxyethyl)amino]-N-[3-(methylcarbamoyl)-4-(2- | |
| | methylpropoxy)phenyl]benzamide\|4-{[3-(2-phenylethynyl)phenyl]methyl}-1H- | |
| | imidazole\|Tuxobertinib\|(S)-4-Amino-N-((2S,3R)-1-(((S)-3-amino-1-oxo-1- | |
| | (((3S,6S,9S,12S,15R,18S,21S)-6,9,18-tris(2-aminoethyl)-15-benzyl-3-((R)-1-hydroxyethyl)-12- | |
| | isobutyl-2,5,8,11,14,17,20-heptaoxo-1,4,7,10,13,16,19-heptaazacyclotricosan-21- | |
| | yl)amino)propan-2-yl)amino)-3-hydroxy-1-oxobutan-2-yl)-3-(3- | |
| | chlorophenyl)butanamide\|Variecolin\|H-Lys-Arg-Pro-Hyp-Gly-Igl-Ser-D-BT-OH(JMV1645)\|2- | |
| | Trityl-4,5-dihydro-1,3-oxazole\|5-Hexyl-2-pyrimidin-2-yloxyphenol\|Naquotinib\|Phenyl-(9-phenyl- | |
| | 9H-purin-6-yl)-amine\|(3R,4R)-4-Amino-1-[4-[(3-methoxyphenyl)amino]pyrrolo[2,1-f][1,2,4]triazin- | |
| | 5-yl]methyl]piperidin-3-ol\|Vatalanib\|N-[4-(4-Phenyl-piperazin-1-yl)-butyl]-benzamide\|Pelitinib\|(S)- | |
| | Morpholin-3-ylmethyl (4-((1-(3-fluorobenzyl)-1H-indazol-5-yl)amino)-5-methylpyrrolo[2,1- | |
| | f][1,2,4]triazin-6-yl)carbamate\|Canertinib\|Poziotinib\|Tarloxotinib\|Aggrenox\|Varlitinib\|3-Propyl- | |
| | [1,4]thiazepan-(5E)-ylideneamine\|Nazartinib\|2-(2,3-Dihydro-1,4-benzodioxin-6-yl)-6-[4-[2-(1- | |
| | methylpyrazol-4-yl)ethylamino]piperidin-1-yl]benzonitrile\|Rociletinib\|Leucovorin\|3-[2-[(2S)-2-[4- | |
| | (4-cyclohexylbutylcarbamoyl)-1,3-oxazol-2-yl]pyrrolidine-1-carbonyl]-4-methoxyphenyl]propanoic | |
| | acid\|Neratinib\|Almonertinib\|Icotinib Hydrochloride\|4- | |
| | (Trifluoromethyl)biphenyl\|Merimepodib\|Nitroglycerin\|6-Chloro-N-(2- | |
| | morpholinoethyl)nicotinamide\|Lapatinib\|2-Phenylthiomorpholine\|Erlotinib\|Gefitinib\|Epidermal | |
| | growth factor\|4-[(2,4-Dichloro-5-methoxyphenyl)amino]-6-methoxy-7-{5-[(4-methylpiperazin-1- | |
| | yl)methyl]furan-3-yl}quinoline-3-carbonitrile\|Dacomitinib\|NCU- | |
| | 190Na\|Afatinib\|Vandetanib\|Neratinib maleate\|Sorafenib\|5,6-Bis[(4-fluorophenyl)amino]-1H- | |
| | isoindole-1,3(2H)-dione\|Terazosin\|N-[3-[2-[4-(2-methoxyethoxy)anilino]-7H-pyrrolo[2,3- | |
| | d]pyrimidin-4-yl]oxy]phenyl]prop-2-enamide\|(1,1′-Biphenyl)-2-sulfonamide,N-(3,4-dimethyl-5- | |
| | isoxazolyl)-4′-(2-oxazolyl)-\|2,5-Dimethoxy-4-ethylamphetamine\|2-(1-Azepanylmethyl)-4-{1-[3-(1- | |
| | azepanylmethyl)-4-hydroxyphenyl]-1-methylethyl}phenol\|(4-Methylphenyl)-(10- | |
| | phenothiazinyl)methanone\|3-(3-Hydroxy-4-methoxyphenyl)-8h-thieno[2,3-b]pyrrolizin-8- | |
| | one\|Zelandopam hydrochloride\|ethyl 6-ethyl-2-methyl-4-oxo-1H-quinoline-3-carboxylate\|1-(((Z)- | |
| | (1-(2-aminothiazol-4-yl)-2-oxo-2-(((3S,4R)-2-oxo-4-((2-oxooxazolidin-3-yl)methyl)-1- | |
| | sulfoazetidin-3-yl)amino)ethylidene)amino)oxy)cyclopropanecarboxylic acid\|2-(2,4- | |
| | Dichlorophenoxy)-5-ethylphenol\|N-(1,4-dioxonaphthalen-2-yl)benzamide | |
| PIM3 | Benzo[c]-2,6-naphthyridin-5-amine,N-(2-chlorophenyl)-7-(1H-1,2,4-triazol-5-yl)-\|N-[5-[3-cyano- | Inhibitor |
| | 7-(cyclopropylamino)pyrazolo[1,5-a]pyrimidin-5-yl]amino]-2- | |
| | methylphenyl]acetamide\|Spiro[piperidine-4,3′-[3H]pyrido[3,4-b]indole]-4′-carbonitrile,7′,8′- | |
| | dichloro-1′,2′,4′,9′-tetrahydro-9′-methyl-1′-oxo-\|7-Bromo-1,10-dihydropyrrolo[2,3- | |
| | a]carbazole\|1,10-Dihydropyrrolo[2,3-a]carbazole-3-carboxamide\|7-(2,4-Difluorophenyl)-1,10- | |
| | dihydropyrrolo[2,3-a]carbazole-3-carbaldehyde\|5-[3-(6-cyclopentyloxypyridin-2-yl)-1H-indol-5-yl]- | |
| | 1,3,4-thiadiazol-2-amine\|5-[3-(6-morpholin-4-ylpyrazin-2-yl)-1H-indol-5-yl]-1,3,4-thiadiazol-2- | |
| | amine\|5-[3-[6-(cyclohexylamino)pyrazin-2-yl]-1H-indol-5-yl]-1,3,4-thiadiazol-2-amine\|6-(6- | |
| | Ethynylpyrazin-2-yl)-1-(6-piperazin-1-ylpyridin-2-yl)pyrazolo[4,3-c]pyridine\|5-amino-2-(2,6- | |
| | difluorophenyl)-N-(1-methyl-1H-pyrazol-4-yl)thiazole-4-carboxamide\|N-[4-(3-aminocyclohexen-1- | |
| | yl)pyridin-3-yl]-2-(2,6-difluorophenyl)quinazolin-8-amine\|N-ethyl-6-[1-(6-piperazin-1-ylpyridin-2- | |
| | yl)pyrazolo[4,3-c]pyridin-6-yl]pyrazin-2-amine\|N-[4-[(3S)-3-aminopiperidin-1-yl]pyrimidin-5-yl]-2- | |
| | (2,6-difluorophenyl)quinazolin-8-amine\|(3R,4S)-3-amino-1-[3-[2-(2,6-difluorophenyl)quinazolin- | |
| | 8-yl]amino]pyridin-4-yl]piperidin-4-ol\|N-[4-[(3S)-3-aminopiperidin-1-yl]pyridin-3-yl]-2-(2,6- | |
| | difluorophenyl)-1,3-thiazole-4-carboxamide\|(S)-1-(6-(3-methylpiperazin-1-yl)pyridin-2-yl)-6-(5- | |
| | (oxetan-3-yl)pyridin-3-yl)-1H-pyrazolo[4,3-c]pyridine\|(S)-5-amino-N-(5-(3-aminopiperidin-1-yl)- | |
| | 1H-pyrazol-4-yl)-2-(2,6-difluorophenyl)thiazole-4-carboxamide\|N-[4-[(3R,4R)-3-amino-4- | |
| | fluoropiperidin-1-yl]pyridin-3-yl]-2-(2,6-difluorophenyl)-1,3-thiazole-4-carboxamide\|N-{4- | |
| | [(3R,4R,5S)-3-Amino-4-hydroxy-5-methylpiperidin-1-yl]pyridin-3-yl}-7-(tetrahydro-2H-pyran-4- | |
| | yl)quinoline-2-carboxamide\|(S)-5-amino-N-(5-(3-aminopiperidin-1-yl)-1-methyl-1H-pyrazol-4-yl)- | |
| | 2-(2-fluorophenyl)thiazole-4-carboxamide\|N-[4-[(3R,4R)-3-amino-4-hydroxypiperidin-1-yl]pyridin- | |
| | 3-yl]-2-(2,6-difluorophenyl)-1,3-thiazole-4-carboxamide\|5-amino-2-(2,6-difluorophenyl)-N-(1- | |
| | methyl-5-(piperidin-4-ylmethylamino)-1H-pyrazol-4-yl)thiazole-4-carboxamide\|3-amino-N-[4- | |
| | [(1R,3R,4R,5S)-3-amino-4-hydroxy-5-methylcyclohexyl]pyridin-3-yl]-6-cyclohexylpyridine-2- | |
| | carboxamide\|N-{4-[(3R,4R,5S)-3-Amino-4-hydroxy-5-methylpiperidin-1-yl]pyridin-3-yl}-7- | |
| | morpholin-4-yl-1,5-naphthyridine-2-carboxamide\|N-{4-[(3r,4r,5s)-3-Amino-4-Hydroxy-5- | |
| | Methylpiperidin-1-Yl]pyridin-3-Yl}-6-(2,6-Difluorophenyl)-5-Fluoropyridine-2-Carboxamide\|3- | |
| | amino-N-[4-[(3R,4R,5S)-3-amino-4-hydroxy-5-methylpiperidin-1-yl]pyridin-3-yl]-6-(2,6- | |
| | difluorophenyl)-5-fluoropyridine-2-carboxamide\|10-methyl-N-(oxan-4-yl)-5-[3- | |
| | (trifluoromethoxy)phenyl]-13-oxa-3,4,6,7,10-pentazatricyclo[7.4.0.02,6]trideca-1(9),2,4,7-tetraen- | |
| | 8-amine\|N-(4-((1R,3R,4S,5S)-3-amino-4-(2-hydroxyethylsulfonyl)-5-methylcyclohexyl)pyridin-3- | |
| | yl)-6-(2,6-difluorophenyl)-5-fluoropicolinamide\|N-(cyclopropylmethyl)-10-methyl-5-[3- | |
| | (trifluoromethoxy)phenyl]-13-oxa-3,4,6,7,10-pentazatricyclo[7.4.0.02,6]trideca-1(9),2,4,7-tetraen- | |
| | 8-amine\|10-methyl-N-(1-methylpiperidin-4-yl)-5-[3-(trifluoromethoxy)phenyl]-13-oxa-3,4,6,7,10- | |
| | pentazatricyclo[7.4.0.02,6]trideca-1(9),2,4,7-tetraen-8-amine\|N-[4-[(1R,3R,4R,5S)-3-amino-4- | |
| | methoxy-5-methylcyclohexyl]pyridin-3-yl]-6-[2,6-difluoro-4-(4-hydroxyoxan-4-yl)phenyl]-5- | |
| | fluoropyridine-2-carboxamide\|U.S. Pat No. 8,889,696, Staurosporine\|methyl N-[(3R,4S,5S)-3-amino-1-[3- | |
| | [[3-amino-6-[2-fluoro-5-(propan-2-ylcarbamoyl)phenyl]pyridine-2-carbonyl]amino]pyridin-4-yl]-5- | |
| | methylpiperidin-4-yl]carbamate | |
| RET | 2-Amino-5,6-dihydro-4H-1,3-thiazine\|Sitravatinib\|Sulochrin \|Citicoline\|5-((5-(4-((4-(2- | Inhibitor\| |
| | hydroxyethyl)piperazin-1-yl)methyl)-3-(trifluoromethyl)phenylcarbamoyl)-2- | Modulator |
| | methylphenyl)ethynyl)-N,1-dimethyl-1H-imidazole-2-carboxamide\|(S)-5-fluoro-2-(1-(5- | |
| | fluoropyridin-2-yl)ethylamino)-6-(5-methyl-1H-pyrazol-3-ylamino)nicotinonitrile\|1-(4-((4- | |
| | Ethylpiperazin-1-yl)methyl)-3-(trifluoromethyl)phenyl)-3-(4-((6-(methylamino)pyrimidin-4- | |
| | yl)oxy)phenyl)urea\|Unii-3mae0mxi6K\|(Z)-3-((1H-pyrrol-2-yl)methylene)indolin-2-one\|(E)-3-(4- | |
| | hydroxybenzylidene)indolin-2-one\|(3Z)-5-Amino-3-[(4-methoxyphenyl)methylidene]-1,3-dihydro- | |
| | 2H-indol-2-one\|(15S,16S)-16-(hydroxymethyl)-16-methoxy-15-methyl-28-oxa-4,14,19- | |
| | triazaoctacyclo[12.11.2.115,18.02,6.07,27.08,13.019,26.020,25]octacosa- | |
| | 1,6,8,10,12,20,22,24,26-nonaen-3-one\|Tamatinib\|2-(4-(4-ethoxy-6-oxo-1,6-dihydropyridin-3-yl)- | |
| | 2-fluorophenyl)-N-(5-(1,1,1-trifluoro-2-methylpropan-2-yl)isoxazol-3-yl)acetamide\|Zeteletinib | |
| | hemiadipate\|Agerafenib\|Methyl Pyridazine-4-carboxylate\|(2S)-2-[4-[5-[6-[(6-methoxypyridin-3- | |
| | yl)methyl]-3,6-diazabicyclo[3.1.1]heptan-3-yl]pyrazin-2-yl]-3-methylpyrazolo[1,5-a]pyridin-6- | |
| | yl]oxymethyl]morpholine\|Pralsetinib\|Semaxanib\|Regorafenib\|Vandetanib\|Ponatinib\|N-benzyl-4- | |
| | (5-formyl-6-hydroxynaphthalen-2-yl)-N-methylbenzamide\|Tupichinol C\|Belfosdil\|1-(Phenyl(p- | |
| | tolyl)methylene)thiosemicarbazide\|6-Methoxyluteolin\|Benzo(b)cyclobuta(d)thiophene-2,2a(7bH)- | |
| | dicarboxylic acid,1-(1-pyrrolidinyl)-,dimethyl ester\|Tranexamic acid\|Allyl 4- | |
| | (aminosulfonyl)benzoate\|3-Hydroxymethyl-beta-carboline\|Milacemide\|Terpin hydrate\|N-[1-[1-[1- | |
| | [2-[1-[1-[(1-amino-4-methyl-1-oxopentan-2-yl)amino]-4-methyl-1-oxopentan-2-yl]amino]-3-(1H- | |
| | imidazol-5-yl)-1-oxopropan-2-yl]amino]-2-oxoethyl]-2-oxoazepan-3-yl]amino]-1-oxopropan-2- | |
| | yl]amino]-3-(1H-indol-3-yl)-1-oxopropan-2-yl]-2-[3-(4- | |
| | hydroxyphenyl)propanoylamino]pentanediamide\|Molephantin\|trans-3-ACPBPA\|H-DL-Tyr(2,6- | |
| | diMe)-DL-Tic-DL-Lys(Cbz)-NHPh\|6-amino-N-(2,4- | |
| | dichlorobenzyl)nicotinamide\|[(2R,3S,4S,5R,6R)-2-[(2S,3S,4S,5S,6S)-2-[3-[5-[1-[[2-[4-[4-[4- | |
| | amino-6-[3-(4-aminobutylamino)propylamino]-6-oxohexyl]carbamoyl]-1,3-thiazol-2-yl]-1,3-thiazol- | |
| | 2-yl]-1-[(2S,3R,4R,5S,6S)-5-amino-3,4-dihydroxy-6-methyloxan-2-yl]oxy-2-hydroxyethyl]amino]- | |
| | 3-hydroxy-1-oxobutan-2-yl]amino]-3-hydroxy-5-oxopentan-2-yl]amino]-2-[6-amino-2-[3-amino-1- | |
| | [(2,3-diamino-3-oxopropyl)amino]-3-oxopropyl]-5-methylpyrimidine-4-carbonyl]amino]-1-(1H- | |
| | imidazol-5-yl)-3-oxopropoxy]-4,5-dihydroxy-6-(hydroxymethyl)oxan-3-yl]oxy-3,5-dihydroxy-6- | |
| | (hydroxymethyl)oxan-4-yl]carbamate\|(R)-N2-{4-[(3- | |
| | chlorobenzyl)oxy]benzyl}alaninamide\|Axitinib\|2,4″-Dimethoxy-5,3″-dipropyl-biphenyl\|3-[2-[4- | |
| | amino-7-(2,2,2-trifluoroethyl)pyrrolo[2,3-d]pyrimidin-5-yl]ethynyl]-N-cyclopropyl-4- | |
| | fluorobenzamide\|N-[2-(3,4-dihydro-2H-quinolin-1-yl)-2-oxoethyl]-2-[3-[(Z)- | |
| | hydroxyiminomethyl]phenyl]carbamoylamino]-N-quinolin-8-ylacetamide\|Tinidazole\|4- | |
| | (Isopropylthio)-2-(trifluoromethyl)benzonitrile\|4-(3-Cyano-4-phenylpyrrol-1-yl)-2-hydroxybenzoic | |
| | acid\|2-(3-Chlorophenyl)histamine\|BGT-226 free base\|2-[4-[3-[2-(2-Chloro-6-fluorophenyl)ethyl- | |
| | [(2,3-dichlorophenyl)carbamoyl]amino]propyl]phenoxy]-2-methylpropanoic acid\|4- | |
| | ((benzo[d]oxazol-2-yl(5-fluoropyridin-2-yl)amino)methyl)-N-hydroxybenzamide | |
| FER | (2R)-2-[5-[6-amino-5-[(1R)-1-[5-fluoro-2-(triazol-2-yl)phenyl]ethoxy]pyridin-3-yl]-4-methyl-1,3- | Inhibitor |
| | thiazol-2-yl]propane-1,2-diol\|(1S,2S,3R,4R)-3-((5-chloro-2-(((S)-1-methoxy-7-morpholino- | |
| | 6,7,8,9-tetrahydro-5H-benzo[7]annulen-2-yl)amino)pyrimidin-4-yl)amino)bicyclo[2.2.1]hept-5- | |
| | ene-2-carboxamide. | |
| MP2K5 | (Z)-3-(((3-((dimethylamino)methyl)phenyl)amino)(phenyl)methylene)-2-oxoindoline-6- | Inhibitor |
| | carboxamide | |
| STK33 | N-[3-methoxy-4-(4-methylpiperazin-1-yl)phenyl]-1-(4-methoxyphenyl)pyrazolo[3,4-b]pyridin-6- | Inhibitor |
| | amine | |
| PDK2 | 4-[(3-Chloro-4-{[(2r)-3,3,3-Trifluoro-2-Hydroxy-2-Methylpropanoyl]amino}phenyl)sulfonyl]-N,N- | Inhibitor |
| | Dimethylbenzamide | |
| FYN | N-(2-chloro-6-methylphenyl)-2-[(2,6-dimethylpyrimidin-4-yl)amino]-1,3-thiazole-5- | Inhibitor |
| | carboxamide\|Triglyme\|N-(3-fluorophenyl)-6,7-dimethoxy-1,4-dihydroindeno[1,2-c]pyrazol-3- | |
| | amine\|7-(4-fluoroanilino)-1-[(1R,3R)-3-hydroxycyclopentyl]-3-(4-methoxyphenyl)-4H- | |
| | pyrimido[4,5-d]pyrimidin-2-one\|2-Isopropoxy-12-(2-hydroxyethyl)-13,14-dihydronaphthol[2,1- | |
| | a]pyrrolo[3,4-c]carbazole-5-one\|2-Methoxy-12-(2-hydroxyethyl)-13,14-dihydronaphthol[2,1- | |
| | a]pyrrolo[3,4-c]carbazole-5-one\|CID 73755245\|Dasatinib | |
| LATS2 | (3R)-6-methoxy-N-[2-[(1-methylpiperidin-4-yl)methoxy]-4-(1H-pyrazol-4-yl)phenyl]-1,2,3,4- | Inhibitor |
| | tetrahydroisoquinoline-3-carboxamide | |
| EPHA5 | 1H-Imidazo[2,1-f]purine-2,4(3H,8H)-dione,7-(5-hydroxy-2-methylphenyl)-8-(2-methoxyphenyl)- | Inhibitor |
| | 1-methyl-\|(2S)-2-[[(4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid | |
| GUC2D | 8-Bromo-4h-[1,2,4]oxadiazolo[3,4-c][1,4]benzoxazin-1-one\|1h-[1,2,4]Oxadiazolo[4,3- | Inhibitor\| |
| | a]quinoxalin-1-one\|Bay 41-2272\|(E,2Z)-4-Ethyl-2-hydroxyimino-5-nitro-hex-3- | Activator\| |
| | enamide\|Vericiguat\|Riociguat | Modulator |
| M3K9 | 3-(1H-indol-5-yl)-5-(4-((4-methylpiperazin-1-yl)methyl)phenyl)-1H-pyrrolo[2,3-b]pyridine\|2- | Inhibitor |
| | Isopropoxy-12-(2-hydroxyethyl)-13,14-dihydronaphthol[2,1-a]pyrrolo[3,4-c]carbazole-5-one\|2- | |
| | Methoxy-12-(2-hydroxyethyl)-13,14-dihydronaphthol[2,1-a]pyrrolo[3,4-c]carbazole-5-one\|3,9- | |
| | Bis((ethylthio)methyl)-K-252a | |
| BRSK1 | 3-[(2R)-1-(dimethylamino)propan-2-yl]oxy-5-[4-methoxy-5-(1-methylpyrazol-4-yl)pyridin-2- | Inhibitor |
| | yl]amino]pyrazine-2-carbonitrile | |
| CSF1R | N-[3-[2-(trifluoromethoxy)phenyl]methylcarbamoyl]phenyl]-2- | Antagonist\| |
| | (trifluoromethyl)benzamide\|Menthol\|4-Hexyl-5-methylbenzene-1,3-diol\|Usistapide\|Csf1R-IN- | Inhibitor\|. |
| | 2\|Simufilam\|Foslinanib\|Tpi-287\|(S)-2-Methyl-1-[(4-methyl-5-isoquinoline)sulfonyl]- | |
| | homopiperazine\|Vimseltinib\|4-((2-(((1R,2R)-2-hydroxycyclohexyl)amino)benzo[d]thiazol-6- | |
| | yl)oxy)-N-methylpicolinamide\|3-(3-Benzylnaphthalen-2-yl)pyridine\|Sulfatinib\|Pexidartinib\|H-DL- | |
| | Tyr(2,6-diMe)-DL-Tic-DL-Lys(Cbz)-NHPh\|2,4″-Dimethoxy-5,3″-dipropyl-biphenyl\|N-[2-(3,4- | |
| | dihydro-2H-quinolin-1-yl)-2-oxoethyl]-2-[3-[(Z)-hydroxyiminomethyl]phenyl]carbamoylamino]-N- | |
| | quinolin-8-ylacetamide\|Tinidazole\|2-(3-Chlorophenyl)histamine\|5-(3-Methoxy-4-((4- | |
| | methoxybenzyl)oxy)benzyl)pyrimidine-2,4-diamine\|4-cyano-N-(5-(1-(2- | |
| | (dimethylamino)acetyl)piperidin-4-yl)-2′,3′,4′,5′-tetrahydro-[1,1′-biphenyl]-2-yl)-1H-imidazole-2- | |
| | carboxamide\|1-(4-((6,7-Dimethoxyquinolin-4-yl)oxy)-2-methoxyphenyl)-3-(1-(thiazol-2- | |
| | yl)ethyl)urea\|3-Phenyl-1H-benzofuro[3,2-c]pyrazole | |
| DYRK4 | leucettine L41 | Inhibitor |
| KAPCA | N-(2-(4-Bromocinnamylamino)ethyl)-5-isoquinolinesulfonamide\|(R,S)-1-(5-methyl-1H-indol-1- | Inhibitor\|. |
| | yl)propan-2-amine\|TCS JNK 5a\|N-[4-[5-(dimethylamino)pentanoylamino]phenyl]carbamothioyl]- | |
| | 4-propan-2-ylbenzamide\|N-(14-methylallyl)norgalanthamine\|4-(2-Aminoethyl)-2- | |
| | ethylphenol\|Pyridine,3-(methoxymethyl)-5-[(2-methyl-4-thiazolyl)ethynyl]-\|7-Isopropyl-7H- | |
| | adenine\|(1R,6S)-1-(3,4-Dichlorophenyl)-6-(methoxymethyl)-4- | |
| | azabicyclo(4.1.0)heptane\|Trisnorsqualene difluoromethylidene\|1,2-Ethanedione,1,2-di-10h- | |
| | phenothiazin-10-yl-\|(S)-9-(2,3-Dihydroxypropyl)adenine\|1-Deoxy-1-thio-heptaethylene | |
| | glycol\|(3z)-N,N-Dimethyl-2-Oxo-3-(4,5,6,7-Tetrahydro-1h-Indol-2-Ylmethylidene)-2,3-Dihydro-1h- | |
| | Indole-5-Sulfonamide\|3-(2-Dimethylamino-ethyl)-2-methyl-1H-indol-4-ol\|Almotriptan\|Thiamylal\|3- | |
| | (4-Phenyl-6-thiophen-2-ylpyridin-2-yl)phenol\|1-(2-Adamantyl)-3-benzylpyrrolidin-2-one\|(2S)-N- | |
| | [(2S)-1-[(2S)-1-[2-[(2S)-4-amino-1-[(2S)-5-amino-1-[[(2S)-1-[[(2S)-1-[(2S)-1-[2-[[(2S)-1-[(2S)- | |
| | 1-[(2S)-1-amino-4-methylsulfanyl-1-oxobutan-2-yl]amino]-4-methyl-1-oxopentan-2-yl]amino]-3- | |
| | (1H-imidazol-4-yl)-1-oxopropan-2-yl]amino]-2-oxoethyl]amino]-3-methyl-1-oxobutan-2-yl]amino]- | |
| | 1-oxopropan-2-yl]amino]-3-(1H-indol-3-yl)-1-oxopropan-2-yl]amino]-1,5-dioxopentan-2-yl]amino]- | |
| | 1,4-dioxobutan-2-yl]amino]-2-oxoethyl]amino]-3-(4-hydroxyphenyl)-1-oxopropan-2-yl]amino]-5- | |
| | (diaminomethylideneamino)-1-oxopentan-2-yl]-2-[(5-oxopyrrolidine-2- | |
| | carbonyl)amino]pentanediamide\|Tazemetostat\|4″-(1,1-Dimethyl-heptyl)-3,5-dimethyl- | |
| | biphenyl\|N1,N3-bis(3-methoxybenzyl)isophthalamide\|(2S)-2-amino-4-({[4- | |
| | (carboxymethoxy)phenyl](hydroxy)methyl}(hydroxy)phosphoryl)butanoic | |
| | acid\|Prostephabyssine\|#N/A\|7-(4-fluoroanilino)-1-[(1R,3R)-3-hydroxycyclopentyl]-3-(4- | |
| | methoxyphenyl)-4H-pyrimido[4,5-d]pyrimidin-2-one\|1-tert-butyl-3-(naphthalen-1-ylmethyl)-1H- | |
| | pyrazolo[3,4-d]pyrimidin-4-amine\|Balanol\|(3RS,4R)-4-{9-Isopropyl-6-[(pyridin-3-ylmethyl)-amino]- | |
| | 9H-purin-2-ylamino}-hexan-3-ol\|(3RS,4R)-4-{9-Isopropyl-6-[(pyridin-3-ylmethyl)-amino]-9H-purin- | |
| | 2-ylamino}-2-methyl-hexan-3-ol\|(3RS,4R)-4-{9-Isopropyl-6-[(pyridin-3-ylmethyl)-amino]-9H-purin- | |
| | 2-ylamino}-2,2-dimethyl-hexan-3-ol | |
| BUB1 | 2-[1-[(4-ethoxy-2,6-difluorophenyl)methyl]-5,6-dihydro-4H-cyclopenta[c]pyrazol-3-yl]-5-methoxy- | Inhibitor |
| | N-pyridin-4-ylpyrimidin-4-amine\|4-[[2-[1-[(2-fluorophenyl)methyl]-5,6-dihydro-4H- | |
| | cyclopenta[c]pyrazol-3-yl]-5-(2-methylsulfonylethoxy)pyrimidin-4-yl]amino]pyridine-3- | |
| | carboxamide\|5-[2-(dimethylamino)ethoxy]-2-[1-[(4-ethoxy-2,6-difluorophenyl)methyl]-5,6-dihydro- | |
| | 4H-cyclopenta[c]pyrazol-3-yl]-N-pyridin-4-ylpyrimidin-4-amine\|[3-[2-[1-[(4-ethoxy-2,6- | |
| | difluorophenyl)methyl]-5,6-dihydro-4H-cyclopenta[c]pyrazol-3-yl]-4-(pyridin-4-ylamino)pyrimidin- | |
| | 5-yl]oxymethyl]oxetan-3-yl]methanol\|U.S. Pat No. 9,765,058, Example 2-2-10\|U.S. Pat No. 9,765,058, | |
| | Example 2-2-8\|U.S. Pat No. 9,765,058, Example 2-2-9 | |
| CDK5 | Kenpaullone\|4-(7-butyl-5H-pyrrolo[2,3-b]pyrazin-6-yl)phenol\|aminopurvalanol A\|1H-Pyrazolo[3,4- | Inhibitor |
| | b]quinoxalin-3-amine\|4-(6-Cyclohexylmethoxy-9H-purin-2-ylamino)-N,N- | |
| | diethylbenzamide\|#N/A\|(S)-4-(2-(4-Amino-1,2,5-oxadiazol-3-yl)-1-ethyl-7-(piperidin-3- | |
| | ylmethoxy)-1H-imidazo[4,5-c]pyridin-4-yl)-2-methylbut-3-yn-2- | |
| | ol\|Alsterpaullone\|Hymenialdisine\|Indirubin-3′-monoxime\|Indirubin-5-sulphonate\|SU9516\|1- | |
| | Azakenpaullone\|Thieno analogue of kenpaullone\|Manzamine A\|1-Phenyl-3-[2-(pyridin-4-yl)-1,3- | |
| | thiazol-4-yl]urea\|3-(2-Phenyl-1,3-thiazol-4-yl)-1,2-dihydroquinolin-2-one\|Purvalanol | |
| | B\|Olomoucine\|3-[5-Fluoro-4-[4-methyl-2-(methylamino)-1,3-thiazol-5-yl]pyrimidin-2- | |
| | yl]amino]benzenesulfonamide\|Tyr-Pro-Phe-Phe-OC(CH3)3\|8-Methyl-2-morpholin-4-yl-7- | |
| | phenoxychromen-4-one\|3,5,7-Trihydroxy-2-(4-hydroxyphenyl)-8-(3,4,5-trihydroxy-6-methyloxan- | |
| | 2-yl)oxychromen-4-one\|3-(2-aminopyrimidin-4-yl)-5-chloro-1H-indol-2-ol\|5,6,7-Tribromo-2- | |
| | piperazin-1-yl-1,3-diazatricyclo[6.3.1.04,12]dodeca-2,4,6,8(12)-tetraene\|2-N-(4- | |
| | aminocyclohexyl)-9-cyclopentyl-6-N-[[6-(furan-2-yl)pyridin-3-yl]methyl]purine-2,6-diamine | |
| MAPK2 | (10R)-10-methyl-3-(6-methylpyridin-3-yl)-9,10,11,12-tetrahydro-8H- | Inhibitor |
| | [1,4]diazepino[5′,6′:4,5]thieno[3,2-f]quinolin-8-one\|MK2a Inhibitor\|2-(2-Quinolin-3-Ylpyridin-4-Yl)- | |
| | 1,5,6,7-Tetrahydro-4h-Pyrrolo[3,2-C]pyridin-4-One\|2-[3,4-Dioxo-2-(pyridin-4- | |
| | ylamino)cyclobuten-1-yl]amino]-2-(3-hydroxyphenyl)acetamide\|8-(4-Hydroxyphenyl)-2- | |
| | morpholin-4-ylchromen-4-one\|Cdc25C phosphatase (211-221) | |
| CLK3 | Ethyl 3-[(E)-2-Amino-1-Cyanoethenyl]-6,7-Dichloro-1-Methyl-1h-Indole-2-Carboxylate\|leucettine | Inhibitor |
| | L41 | |
| NUAK1 | N-(3-((5-chloro-2-((2-methoxy-4-(4-methylpiperazin-1-yl)phenyl)amino)pyrimidin-4- | Inhibitor |
| | yl)oxy)phenyl)propionamide\|(1S,2S,3R,4R)-3-((5-chloro-2-(((S)-1-methoxy-7-morpholino-6,7,8,9- | |
| | tetrahydro-5H-benzo[7]annulen-2-yl)amino)pyrimidin-4-yl)amino)bicyclo[2.2.1]hept-5-ene-2- | |
| | carboxamide. | |
| CDK3 | Rgb-286638\|2-(5,7-dichloro-1H-indol-3-yl)ethanamine\|4-[4-[2-[5-[(5-tert-butyl-1,3-oxazol-2- | Inhibitor |
| | yl)methylsulfanyl]-1,3-thiazol-2-yl]amino]-2-oxoethyl]phenoxy]-N-hydroxybutanamide\|2-(4-(bis(2- | |
| | chloroethyl)amino)phenyl)-N-(5-((5-tert-butyloxazol-2-yl)methylthio)thiazol-2-yl)acetamide | |
| KITM | Mobocertinib\|1-[(Z)-4-(triphenylmethoxy)-2-butenyl]thymine\|1-[6- | Replacement\| |
| | (Triphenylmethoxy)hexyl]thymine\|1-[5-(Triphenylmethoxy)pentyl]thymine | Inhibitor |
| JAK3 | Azumamide B\|{(3r,4r)-4-Methyl-3-[methyl(7h-Pyrrolo[2,3-D]pyrimidin-4-Yl)amino]piperidin-1- | Inhibitor\| |
| | Yl}(Pyrrolidin-1-Yl)methanone\|(z)-5-(2,4-Dihydroxybenzylidene)thiazolidine-2,4-dione\|Pyridone | Modulator |
| | 6\|2-Bromo-4-[(6,7-dimethoxyquinazolin-4-yl)amino]phenol\|3-[Benzyl(propan-2-yl)amino]-1- | |
| | (naphthalen-2-yl)propan-1-one\|2-N-((1S)-1-(5-Fluoropyrimidin-2-yl)ethyl)-4-N-(1-methylimidazol- | |
| | 4-yl)-7H-pyrrolo(2,3-d)pyrimidine-2,4-diamine\|(S)-5-fluoro-2-(1-(5-fluoropyridin-2-yl)ethylamino)- | |
| | 6-(5-methyl-1H-pyrazol-3-ylamino)nicotinonitrile\|N-(2-chloro-6-methylphenyl)-2-[(2,6- | |
| | dimethylpyrimidin-4-yl)amino]-1,3-thiazole-5-carboxamide\|2,12- | |
| | Bis[(dimethylamino)methyl]cyclododecanone\|6-o-Tolylquinazolin-2-amine\|Atropisomer 1\|3-Ethyl- | |
| | 4-methylpyridine\|(2Z)-3-phenylmethoxy-5-(1H-pyrrol-2-yl)-2-[(5-undecyl-1H-pyrrol-2- | |
| | yl)methylidene]pyrrole; hydrochloride\|Tyrphostin B42\|Thiourea,N-(5-chloro-2-pyridinyl)-N′-(2-(3- | |
| | cyano-2,6-difluorophenyl)ethyl)-\|Cerdulatinib\|Ifidancitinib\|Simufilam\|2-Propen-1-one,1-[(2S,5R)- | |
| | 2-methyl-5-(7H-pyrrolo[2,3-d]pyrimidin-4-ylamino)-1-piperidinyl]- | |
| | \|Decernotinib\|Peficitinib\|Tofacitinib\|N-(6-phenyl-1H-indazol-3-yl)butyramide\|Cdk7-IN-2\|5,8- | |
| | dichloro-7-[1-(2-hydroxyacetyl)piperidin-4-yl]-methoxymethyl]-2-[(4-methoxy-6-methyl-2-oxo-1H- | |
| | pyridin-3-yl)methyl]-3,4-dihydroisoquinolin-1-one\|13,16,19,22-Tetraoxa-3,6- | |
| | diazatricyclo[21.3.1.18,12]octacosa-1(27),2,6,8(28),9,11,23,25-octaene-27,28-diol\|2-Methoxy-N- | |
| | n-propylnorapomorphine\|Nicotinamide riboside\|(3-(4-(6-Oxa-2-azaspiro[3.4]octan-2-ylmethyl)-3- | |
| | methylphenoxy)azetidin-1-yl)(5-phenyl-1,3,4-oxadiazol-2-yl)methanone\|1-Cyclohexyl-4-[3-(5- | |
| | methoxy-1,2,3,4-tetrahydronaphthalen-1-yl)propyl]piperazine\|Fluspirilene\|Enadoline\|3-(2- | |
| | (Pyridin-4-yl)ethyl)-1H-indole\|7-(3-Methoxyphenyl)-2-methylquinoline\|Indiplon\|8-Bromo-3-methyl- | |
| | 5-phenyl-2,3,4,5-tetrahydro-1H-benzo[d]azepin-7-ol\|Acetic acid 2-heptylselanyl-phenyl ester\|4- | |
| | [3-(2-Fluorophenoxy)phenyl]-1-phosphonatobutane-1-sulfonate\|5-Butyl-8-phenyl-3H- | |
| | [1,2,4]triazolo[5,1-i]purine\|Tyr-Pro-Phe-Hfe-NH2\|2-[4-(3-Methyl-2-thienyl)phenyl]propionic | |
| | acid\|Crenigacestat\|Azeloyl diethyl salicylate\|1-[5-Ethyl-2-hydroxy-4-[6-methyl-6-(1H-tetrazol-5- | |
| | YL)heptyl]oxy]phenyl]ethanone\|Moxidectin\|Rhein\|Cyclofenil\|7 alpha,27- | |
| | Dihydroxycholesterol\|deamino-Cys(1)-nTyr-D-xille-D-Gln-D-Asn-D-Cys(1)-D-Pro-Lys(Unk)- | |
| | Unk\|Loviride\|4-(2-amino-2-iminoethyl)-1-(2-chlorophenyl)-5-(4-chlorophenyl)-N-(4-fluorophenyl)- | |
| | 1H-pyrazole-3-carboxamide\|1-(3-carbamimidoylphenyl)-3-methyl-N-[4-(2- | |
| | sulfamoylphenyl)phenyl]-1H-pyrazole-5-carboxamide\|[(2S)-1-[(2R)-2-boronopyrrolidin-1-yl]-1- | |
| | oxopropan-2-yl]azanium\|Gccsnpvchlehsnlc*\|N-(5-fluoropyridin-2-yl)-6-(5-fluoropyridin-3-yl)oxy- | |
| | [1,2,4]triazolo[4,3-a]pyridine-8-carboxamide\|6H-(1,2,4)Triazolo(4,3-a)(1,5)benzodiazepine-6- | |
| | acetamide, 4,5-dihydro-4-(1H-indol-3-ylmethyl)-N-(1-methylethyl)-5-oxo-1-phenyl-N- | |
| | (phenylmethyl)-, (S)-\|N-(5-(3,5-difluorophenylthio)-1-H-pyrazolo[3,4-b]pyridine-3-yl)-4-(4- | |
| | methylpiperazine-1-yl)-2-(tetrahydro-2H-pyran-4-ylamino)benzamide\|Sardomozide\|(5-(3- | |
| | methoxyphenyl)-1-pentyl-1H-pyrrol-3-yl)(naphthalen-1-yl)methanone\|N-Cyclopropyl-5-[2-Methyl- | |
| | 5-(Trifluoromethoxy)-1h-Benzimidazol-1-Yl]thiophene-2-Carboxamide\|Rilzabrutinib\|1-(2-Ethoxy- | |
| | 1-phenylethyl)-4-phenylpiperidin-4-ol\|N-[(1S)-3-[(2Z)-2-[(4R)-3,4-Dimethyl-1,3-thiazolidin-2- | |
| | ylidene]hydrazinyl]-1-(oxan-4-yl)-2,3-dioxopropyl]cycloheptanecarboxamide\|8- | |
| | Methyltheophylline\|1,4-Dihydroxyanthraquinone\|4-(3- | |
| | Nitrobenzylideneamino)Benzenesulfonamide\|Jnk-IN-8\|N,N′-Bis(3-(ethylamino)propyl)-1,7- | |
| | heptanediamine\|2-[4-[2-(diethylamino)ethoxy]phenyl]-N-[(2,4-difluorophenyl)methyl]-5- | |
| | fluoropyrimidin-4-amine\|ENS-163 phosphate\|Rjf24P5qds\|Balapiravir\|3-Hydroxybutan-2-yl 6,11- | |
| | dimethyl-6,11-diazatetracyclo[7.6.1.0\hat{ }{2,7}.0\hat{ }{12,16}]hexadeca-1(16),9,12,14-tetraene-4- | |
| | carboxylate\|1-(5-Bromo-2-pyridyl)-3-[2-(3-fluorophenyl)ethyl]thiourea\|(2S)-N-dodecyl-1-[(2S)- | |
| | pyrrolidine-2-carbonyl]pyrrolidine-2-carboxamide\|H-Gly-Gly-Thr-Thr-Gly-Gly-Thr-Gly-Thr-Gly- | |
| | Gly-Thr-Thr-Gly-Gly-OH\|4-[4-(Benzyloxy)piperidino]butyl-4-fluorobenzoate\|Vicriviroc\|2-(3- | |
| | Acrylamidophenyl)-N-(tert-butyl)-5H-pyrrolo[2,3-b]pyrazine-7-carboxamide\|H-D-Cys(Bzl)-OH\|3- | |
| | (1-adamantyl)-6,7,8,9-tetrahydro-5H-[1,2,4]triazolo[4,3-a]azepine\|3-(4-chlorophenyl)-N-(pyridin- | |
| | 4-ylmethyl)adamantane-1-carboxamide\|8-(1,3-oxazol-5-yl)-N-phenyl-6- | |
| | (trifluoromethyl)imidazo[1,2-a]pyridine-3-carboxamide\|Bifeprunox\|Methyl piperate\|2- | |
| | Hydrazinylbenzenesulfonamide\|[(4R,4aR,7S,7aR,12bS)-9-acetyloxy-3-methyl- | |
| | 1,2,3,4,4a,7,7a,13-octahydro-4,12-methanobenzofuro[3,2-e]isoquinolin-3-ium-7-yl]sulfate | |
| MK03 | 6-[(E)-2-(4-fluorophenyl)ethenyl]-7H-purine\|1-(((Z)-(1-(2-aminothiazol-4-yl)-2-oxo-2-(((3S,4R)-2- | Inhibitor\| |
| | oxo-4-((2-oxooxazolidin-3-yl)methyl)-1-sulfoazetidin-3- | Activator\| |
| | yl)amino)ethylidene)amino)oxy)cyclopropanecarboxylic acid\|6-(3′,5′-Dimethylbenzyl)-1- | Modulator |
| | ethoxymethyl-5-isopropyluracil\|Ravoxertinib\|4-(4-methoxy-1H-indol-1- | |
| | ylsulfonyl)benzenamine\|Cilofexor\|Mitoquinone\|(3S)-3-[(2S)-2-[3-(methoxycarbonylamino)-2- | |
| | oxopyridin-1-yl]butanoyl]amino]-4-oxo-5-(2,3,5,6-tetrafluorophenoxy)pentanoic acid\|Ulixertinib | |
| KGP2 | (3S,4R)-N-(7-chloro-1-oxo-1,2-dihydroisoquinolin-6-yl)-4-(4-chlorophenyl)pyrrolidine-3- | Inhibitor |
| | carboxamide | |
| RAF1 | Rodatristat ethyl\|Belvarafenib\|Semapimod\|(S)-6-(4-Chlorophenyl)-N-(1-hydroxypropan-2-yl)-2- | Inhibitor\| |
| | (1-methyl-1H-pyrazol-4-yl)-3-oxo-2,3-dihydropyridazine-4-carboxamide\|Dadle\|N-[(1S)-1-(5- | Modulator\|. |
| | fluoropyridin-2-yl)ethyl]-3-(3-propan-2-yloxy-1H-pyrazol-5-yl)imidazo[4,5-b]pyridin-5- | |
| | amine\|Tinostamustine\|Dicyclomine\|c[L-Phe-D-pro-L-Tyr(OMe)-D-trp]\|Acetohydroxamic acid\|6- | |
| | Hydroxy-5,7-dimethyl-beta-carboline\|4-[1-(4-Hydroxyphenyl)-2-phenylpent-1- | |
| | enyl]phenol\|Semagacestat\|c[-Arg-Gly-Asp-Acpca19-]\|N-[2-[(2S)-2-cyanopyrrolidin-1-yl]-2- | |
| | oxoethyl]-6-methoxyquinoline-4-carboxamide\|N-benzyl brucine\|Amoxapine\|Formaldehyde\|3- | |
| | (dimethylamino)-N-(3-(4-hydroxybenzamido)-4-methylphenyl)benzamide\|Purine,6-(benzylthio)- | |
| | \|6-[(E)-2-(4-fluorophenyl)ethenyl]-7H-purine\|5-Iodo-3-[(3,5-dibromo-4-hydroxyphenyl)methylene]- | |
| | 2-indolinone\|Debromohymenialdisine\|5-(4-Hydroxy-2,6-dimethylstyryl)nicotinic acid\|4-[3-methyl- | |
| | 2-piperidin-4-yl-5-[3-(trifluoromethyl)phenyl]imidazol-4-yl]-N-[(1S)-1-phenylethyl]pyridin-2- | |
| | amine\|[6-Cyclopropylmethoxy-5-(3,3-difluoro-azetidin-1-yl)-pyridin-2-yl]-(2,2-dimethyl-pyrrolidin- | |
| | 1-yl)-methanone\|8-Prenylquercetin\|Amphotericin b\|Cki27; CK127; CK 127; RG-7304; RG | |
| | 7304\|Naporafenib\|(r)-2-(1-(6-Amino-5-chloropyrimidine-4-carboxamido)ethyl)-n-(5-chloro-4- | |
| | (trifluoromethyl)pyridin-2-yl)thiazole-5-carboxamide | |
| TESK2 | 1-[5-[6-(2,6-Dimethyl-4-pentan-3-yloxyphenyl)-2-pyrazin-2-ylpyrimidin-4-yl]-1,3-thiazol-2-yl]-3- | Inhibitor |
| | methylurea\|1-Ethyl-3-[5-[6-(4-methoxy-2,6-dimethylphenyl)-2-pyrazin-2-ylpyrimidin-4-yl]-1,3- | |
| | thiazol-2-yl]urea\|N-[5-[6-(2,6-dichloro-4-methoxyphenyl)-2-pyrazin-2-ylpyrimidin-4-yl]-1,3-thiazol- | |
| | 2-yl]acetamide | |
| F263 | 3-(3-Pyridinyl)-1-(4-pyridinyl)-2-propen-1-one\|2-Propen-1-one,1-(4-pyridinyl)-3-(7- | Antagonist\| |
| | (trifluoromethyl)-2-quinolinyl)-, (2E)- | Inhibitor |
| KPCT | Phosphoserine\|Phosphonothreonine\|Bisindolylmaleimide IV\|(18S)-18-(hydroxymethyl)-17-oxa- | Inhibitor\|. |
| | 4,14,21-triazahexacyclo[19.6.1.17,14.02,6.08,13.022,27]nonacosa- | |
| | 1(28),2(6),7(29),8,10,12,22,24,26-nonaene-3,5-dione\|Phenol,4,4′-(3-methyl-1-butenylidene)bis- | |
| | \|(E)-3-(4-Hydroxyphenyl)-2,3-diphenylacrylonitrile\|4,4′-(Cycloheptylidenemethylene)diphenol\|2- | |
| | (4-Hydroxyphenyl)-3,3-diphenylacrylonitrile\|3,3-Bis(4-methoxyphenyl)-2- | |
| | phenylpropenenitrile\|2,3,3-Triphenylacrylonitrile\|3,3-Bis(4-hydroxyphenyl)-2-phenylprop-2- | |
| | enenitrile\|Cyclofenil diphenol\|3-(1-Methyl-1H-indol-3-yl)-4-[6,7,8,9-tetrahydro-12-methyl- | |
| | 7beta,8alpha-(methaniminomethano)pyrido[1,2-a]indol]-10-yl]-1H-pyrrole-2,5-dione\|2,2′:5′,2″′- | |
| | Terthiophene-4-carbaldehyde\|2,2′:5′,2″-Terthiophene-4,5″-dicarbaldehyde\|2,2′:5′,2″- | |
| | Terthiophene-4-methanol\|4-Morpholin-4-yl-chromen-2-one\|Sotrastaurin acetate\|17- | |
| | (Hydroxymethyl)-18-oxa-4,14,22-triazahexacyclo[20.6.1.17,14.02,6.08,13.023,28]triaconta- | |
| | 1(29),2(6),7(30),8,10,12,23,25,27-nonaene-3,5-dione\|(S)-3-(8-(Dimethylaminomethyl)-6,7,8,9- | |
| | tetrahydropyrido(1,2-a)indol-10-yl)-4-(1-methyl-3-indolyl)-1H-pyrrole-2,5-dione | |
| | hydrochloride\|Minodronic acid\|Codeinone\|Di-Tert-Butyl {iminobis[(2s,3s)-3-Hydroxy-1- | |
| | Phenylbutane-4,2-Diyl]}biscarbamate\|[(4R,4aR,7S,7aR,12bS)-9-benzoyloxy-3,3-dimethyl- | |
| | 2,4,4a,7,7a,13-hexahydro-1H-4,12-methanobenzofuro[3,2-e]isoquinolin-3-ium-7-yl]sulfate\|Xac- | |
| | BY630\|Prostratin\|8-Octyl-benzolactam-V9\|4-(1H-indol-5-ylamino)-5-styrylnicotinonitrile\|4-(3H- | |
| | indol-4-ylamino)-5-styrylnicotinonitrile\|4-(1H-indol-6-ylamino)-5-styrylnicotinonitrile\|4-(1H-indol-5- | |
| | yloxy)-5-styrylnicotinonitrile | |
| BTK | Orelabrutinib\|4-(tert-Butyl)-N-(2-methyl-3-(4-methyl-6-((4-(morpholine-4-carbonyl)phenyl)amino)- | Inhibitor\| |
| | 5-oxo-4,5-dihydropyrazin-2-yl)phenyl)benzamide\|6-Cyclopropyl-8-fluoro-2-[2-(hydroxymethyl)-3- | Modulator |
| | [1-methyl-5-[5-(4-methylpiperazin-1-yl)pyridin-2-yl]amino]-6-oxopyridin-3-yl]phenyl]isoquinolin-1- | |
| | one\|N-[(1S)-1-(5-fluoropyridin-2-yl)ethyl]-3-(3-propan-2-yloxy-1H-pyrazol-5-yl)imidazo[4,5- | |
| | b]pyridin-5-amine\|Blk-IN-1\|2-methyl-N-[2-({3-[2-(prop-2- | |
| | enamido)acetamido]phenyl}amino)pyrimidin-5-yl]-5-[3- | |
| | (trifluoromethyl)benzamido]benzamide\|Inositol 1,3,4,5-tetrakisphosphate\|(Z)-2-cyano-N-(2,5- | Modulator |
| | dibromophenyl)-3-hydroxybut-2-enamide\|N-[3-[6-[4-[(2R)-1,4-dimethyl-3-oxopiperazin-2- | |
| | yl]anilino]-4-methyl-5-oxopyrazin-2-yl]-2-methylphenyl]-4,5,6,7-tetrahydro-1-benzothiophene-2- | |
| | carboxamide\|N-(3-(3-cyanobenzamido)phenyl)-2-methoxybenzamide\|(Z)-2-[3-[4-amino-5-(4- | |
| | phenoxyphenyl)pyrrolo[2,3-d]pyrimidin-7-yl]piperidine-1-carbonyl]-3-cyclopropylprop-2- | |
| | enenitrile\|(E)-2-[2-[4-amino-5-(4-phenoxyphenyl)pyrrolo[2,3-d]pyrimidin-7-yl]methyl]pyrrolidine- | |
| | 1-carbonyl]-4-(dimethylamino)-4-methylpent-2-enenitrile\|Golotimod\|(4S)-6-Chloro-4- | |
| | cyclopropylethynyl-4-trifluoromethyl-3,4-dihydro-1H-quinazolin-2-one\|5-Amino-N-ethyl-2-methyl- | |
| | N-phenylbenzenesulphonamide\|Taurocholic acid\|4-[(E)-2-(3-benzyl-1,2,4-oxadiazol-5- | |
| | yl)ethenyl]benzene-1,2-diol\|N-(3-(Dimethylamino)propyl)-4-(8-hydroxyquinolin-6-yl)benzamide\|6- | |
| | Chloro-3-isopropylamino-4H-thieno(3,2-E)-1,2,4-thiadiazine 1,1- | |
| | dioxide\|Orladeyo\|Cilofexor\|Avasopasem manganese dipropionate\|Poseltinib\|gamma-Linolenic | |
| | acid\|Vecabrutinib\|Urea,N-(4-(2,3-dihydro-7-(5-methyl-1H-imidazol-2-yl)-1-oxo-1H-isoindol-4-yl)- | |
| | 3-fluorophenyl)-N′-(3-(trifluoromethyl)phenyl)-\|(2-Chloro-4-phenoxyphenyl)(4-(((3R,6S)-6- | |
| | (hydroxymethyl)tetrahydro-2H-pyran-3-yl)amino)-7h-pyrrolo[2,3-d]pyrimidin-5- | |
| | yl)methanone\|Spebrutinib\|Bms-986142\|4alpha-(hydroxymethyl)-4alpha-demethylterritrem | |
| | B\|Birinapant\|Rilzabrutinib\|Tirabrutinib\|Evobrutinib\|3,5-Dithiocyanatopyridine-2,6- | |
| | diamine\|Fenebrutinib\|Pirtobrutinib\|((plusmn))-Zanubrutinib\|\|brutinib\|Acalabrutinib\|(E)-N-[3-[4- | |
| | amino-5-(3-methoxy-4-methylphenyl)pyrrolo[2,3-d]pyrimidin-7-yl]phenyl]-4-(dimethylamino)-N- | |
| | methylbut-2-enamide\|(3R,5R)-7-[4-(4-fluorophenyl)-5-methyl-1-phenyl-2-(propan-2-yl)-1H-pyrrol- | |
| | 3-yl]-3,5-dihydroxyheptanoate\|5-[4-[2-Fluoro-3-[(2-methylpyrimidin-5- | |
| | yl)methoxy]phenoxy]phenyl]-7-propan-2-ylpyrrolo[2,3-d]pyrimidin-4-amine\|(E)-N-[3-[4-amino-5- | |
| | (4-phenoxyphenyl)pyrrolo[2,3-d]pyrimidin-7-yl]phenyl]-4-[cyclopropyl(methyl)amino]-N- | |
| | methylbut-2-enamide\|N-(5-fluoropyridin-2-yl)-6-(5-fluoropyridin-3-yl)oxy-[1,2,4]triazolo[4,3- | |
| | alpyridine-8-carboxamide\|4-(2-amino-2-iminoethyl)-1-(2-chlorophenyl)-5-(4-chlorophenyl)-N-(4- | |
| | fluorophenyl)-1H-pyrazole-3-carboxamide\|Azeloyl diethyl salicylate\|Gccsnpvchlehsnlc*\|1-[3-[5- | |
| | (1-benzylpyrazol-4-yl)-7H-pyrrolo[2,3-d]pyrimidin-4-yl]amino]piperidin-1-yl]prop-2-en-1-one\|(5-(3- | |
| | methoxyphenyl)-1-pentyl-1H-pyrrol-3-yl)(naphthalen-1-yl)methanone\|2-Phenoxy-3-(piperidin-4- | |
| | yl)pyridine\|8-cyclopropyl-4-[2-(hydroxymethyl)-3-[6-[4-[(4-methylpiperazin-1-yl)methyl]phenyl]- | |
| | 7H-pyrrolo[2,3-d]pyrimidin-4-yl]phenyl]-2,3-dihydro-1,4-benzoxazepin-5-one\|4-[4-[5-fluoro-2- | |
| | methyl-3-[(5-methyl-4,5,6,7-tetrahydro-1-benzothiophene-2-carbonyl)amino]phenyl]-7H- | |
| | pyrrolo[2,3-d]pyrimidin-6-yl]-N,N-dimethyl-3,6-dihydro-2H-pyridine-1-carboxamide\|3-[4-amino-7- | |
| | (2,3-dihydro-1H-inden-2-yl)pyrrolo[2,3-d]pyrimidin-5-yl]-5-fluorophenol\|8-cyclopropyl-6-fluoro-4- | |
| | [2-(hydroxymethyl)-3-[6-[1-(1-prop-2-enoylpiperidin-3-yl)pyrazol-4-yl]-7H-pyrrolo[2,3-d]pyrimidin- | |
| | 4-yl]phenyl]-2,3-dihydro-1,4-benzoxazepin-5-one\|6-cyclopropyl-2-[2-(hydroxymethyl)-3-[6-[4-[(4- | |
| | methylpiperazin-1-yl)methyl]phenyl]-7H-pyrrolo[2,3-d]pyrimidin-4-yl]phenyl]phthalazin-1-one\|4- | |
| | amino-N-(5-chloro-1,3-benzoxazol-2-yl)-7-(1-prop-2-enoylpyrrolidin-3-yl)pyrrolo[2,3-d]pyrimidine- | |
| | 5-carboxamide\|4-cyclopropyl-N-[2-methyl-3-[6-[4-(piperazine-1-carbonyl)phenyl]-7H-pyrrolo[2,3- | |
| | d]pyrimidin-4-yl]phenyl]benzamide\|5-tert-butyl-N-[[2-fluoro-4-[6-(1-methylpyrazol-4-yl)-7H- | |
| | pyrrolo[2,3-d]pyrimidin-4-yl]phenyl]methyl]-1,2,4-oxadiazole-3-carboxamide\|(E)-1-[3-[4-amino-5- | |
| | (4-phenoxyphenyl)pyrrolo[2,3-d]pyrimidin-7-yl]pyrrolidin-1-yl]-4-(dimethylamino)but-2-en-1- | |
| | one\|Abivertinib\|N-(3-(5-chloro-2-((3-morpholinophenyl)amino)-7H-pyrrolo[2,3-d]pyrimidin-4- | |
| | yl)phenyl)acrylamide\|8-cyclopropyl-6-fluoro-4-[5-fluoro-2-(hydroxymethyl)-3-[6-[5-[4-(2,2,2- | |
| | trifluoroethyl)piperazin-1-yl]methyl]pyridin-2-yl]-7H-pyrrolo[2,3-d]pyrimidin-4-yl]phenyl]-2,3- | |
| | dihydro-1,4-benzoxazepin-5-one\|5-tert-butyl-N-[[2-methyl-4-[6-[4-(morpholine-4- | |
| | carbonyl)phenyl]-7H-pyrrolo[2,3-d]pyrimidin-4-yl]phenyl]methyl]-1,3-thiazole-2-carboxamide | |
| CDK18 | 2-(2-Aminopyrimidin-4-yl)-4-(2-chloro-4-methoxyphenyl)-1,3-thiazole-5-carboxamide | Inhibitor |
| KS6A2 | (1S,2S,3R,4R)-3-((5-chloro-2-(((S)-1-methoxy-7-morpholino-6,7,8,9-tetrahydro-5H- | Inhibitor |
| | benzo[7]annulen-2-yl)amino)pyrimidin-4-yl)amino)bicyclo[2.2.1]hept-5-ene-2-carboxamide.\|2- | |
| | ((3,5-difluoro-4-hydroxyphenyl)amino)-8-isopentyl-5,7-dimethyl-7,8-dihydropteridin-6(5H)-one | |
| EPHA3 | Indatraline\|1H-Imidazo[2,1-f]purine-2,4(3H,8H)-dione,7-(5-hydroxy-2-methylphenyl)-8-(2- | Antagonist\| |
| | methoxyphenyl)-1-methyl-\|5-((5-(4-((4-(2-hydroxyethyl)piperazin-1-yl)methyl)-3- | Inhibitor\|. |
| | (trifluoromethyl)phenylcarbamoyl)-2-methylphenyl)ethynyl)-N,1-dimethyl-1H-imidazole-2- | |
| | carboxamide\|(2S)-2-[(4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid\|Cobimetinib | |
| EPHA1 | 1H-Imidazo[2,1-f]purine-2,4(3H,8H)-dione,7-(5-hydroxy-2-methylphenyl)-8-(2-methoxyphenyl)- | Inhibitor |
| | 1-methyl-\|(2S)-2-[(4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid | |
| CHK2 | 1-(2-(3-Aminopropoxy)phenyl)-3-(pyrazin-2-yl)urea\|4-Fluoro-2-(4-{[(3s,4r)-4-(1-Hydroxy-1- | Modulator\| |
| | Methylethyl)pyrrolidin-3-Yl]amino}-6,7-Dimethoxyquinazolin-2- | Inhibitor |
| | Yl)phenol\|Chloroxine\|(1S,2S,3R,4R)-3-((5-chloro-2-(((S)-1-methoxy-7-morpholino-6,7,8,9- | |
| | tetrahydro-5H-benzo[7]annulen-2-yl)amino)pyrimidin-4-yl)amino)bicyclo[2.2.1]hept-5-ene-2- | |
| | carboxamide.\|Chk2 Inhibitor II\|2-(4-Phenoxy-phenyl)-1H-benzoimidazol-5-ylamine\|5-Nitro-2-(4- | |
| | phenoxy-phenyl)-1H-benzoimidazole\|Debromohymenialdisine\|Irofulven\|Hydroxyzine | |
| FES | (2R)-2-[5-[6-amino-5-[(1R)-1-[5-fluoro-2-(triazol-2-yl)phenyl]ethoxy]pyridin-3-yl]-4-methyl-1,3- | Inhibitor |
| | thiazol-2-yl]propane-1,2-diol\|(1S,2S,3R,4R)-3-((5-chloro-2-(((S)-1-methoxy-7-morpholino- | |
| | 6,7,8,9-tetrahydro-5H-benzo[7]annulen-2-yl)amino)pyrimidin-4-yl)amino)bicyclo[2.2.1]hept-5- | |
| | ene-2-carboxamide. | |
| KC1G3 | Benzo[c]-2,6-naphthyridin-5-amine,N-(2-chlorophenyl)-7-(1H-1,2,4-triazol-5-yl)- | Inhibitor |
| MAPK5 | 2-(2-Quinolin-3-Ylpyridin-4-Yl)-1,5,6,7-Tetrahydro-4h-Pyrrolo[3,2-C]pyridin-4-One\|4-(8-((4- | Inhibitor |
| | ((1S,4S)-5-Isopropyl-2,5-diazabicyclo(2.2.1)heptan-2-yl)phenyl)amino)-(1,2,4)triazolo(1,5- | |
| | a)pyrazin-5-yl)furan-2-carboxamide | |
| KITH | Rilapladib\|Brivudine\|Thymidine-5′-phosphate\|Thymidine-5′-triphosphate\|p1-(5′-Adenosyl)p5-(5′- | Inhibitor\| |
| | thymidyl)pentaphosphate\|Thymidine\|5-Iodo-2′-deoxyuridine-5′-monophosphate\|9-(4- | Modulator |
| | Hydroxybutyl)-N2-phenylguanine\|6-(Dihydroxy-isobutyl)-thymine\|(R)-9-(2- | |
| | Hydroxypropyl)adenine\|(South)-methanocarba-thymidine\|(S)-1-(6-Amino-9H-purin-9-yl)propan- | |
| | 2-ol\|5-Bromothienyldeoxyuridine\|6-Hydroxypropylthymine\|[(2S,3R,5R)-5-[5-[(E)-2- | |
| | bromoethenyl]-2,4-dioxopyrimidin-1-yl]-3-hydroxyoxolan-2-yl]methyl dihydrogen phosphate\|3- | |
| | Hydroxyandrost-5-en-17-one\|1-[(Z)-4-(triphenylmethoxy)-2-butenyl]thymine\|1-[6- | |
| | (Triphenylmethoxy)hexyl]thymine\|1-[5-(Triphenylmethoxy)pentyl]thymine\|1-[2-(2- | |
| | Triphenylmethoxyethoxy)ethyl]thymine\|1-[7-(Triphenylmethoxy)heptyl]thymine\|3- | |
| | Propargylthymidine\|#N/A\|N(2)-(3-Trifluoromethylphenyl)guanine\|5-Propyl-2′- | |
| | deoxyuridine\|Edoxudine\|Uridine,2′-deoxy-5-iodo-\|L-5-(bromovinyl)deoxyuridine\|3′-Deoxy-3′- | |
| | (1,2,3-triazol-1-yl)thymidine\|2′-Deoxyuridine\|9-[3-(Dimethylamino)propylamino]-9-oxononanoic | |
| | acid\|Mebutamate\|Sigma-2 receptor antagonist 1\|Arginine butyrate\|Tanzisertib\|Valnivudine | |
| | hydrochloride\|Dioxane\|Penciclovir\|2′-Deoxycytidine | |
| SIK2 | 1-(2,4-Dimethoxyphenyl)-3-(2,6-dimethylphenyl)-1-[6-[4-(4-methylpiperazin-1- | Inhibitor |
| | yl)anilino]pyrimidin-4-yl]urea | |
| NEK6 | N-(7-chloro-1-oxo-1,2-dihydroisoquinolin-6-yl)-2-(cyclopropylamino)-2-phenylacetamide | Inhibitor |
| MP2K2 | Prussian blue\|Berzosertib\|7-Fluoro-3-[(2-Fluoro-4-Iodophenyl)amino]-N-{[(2s)-2- | .\|Inhibitor |
| | Hydroxypropyl]oxy}furo[3,2-C]pyridine-2-Carboxamide\|Ruxolitinib\|(Z)-3-(3-(((4- | |
| | ((dimethylamino)methyl)phenyl)amino)(phenyl)methylene)-2-oxoindolin-6-yl)-N- | |
| | ethylpropiolamide\|5-Chloro-2-(4-hydroxy-phenyl)-benzooxazol-6-ol\|Etrabamine\|3-(3- | |
| | Thienyl)quinoline\|Des-AA1,2,5-[IAmp9,Tyr11]-SRIF\|2,3,4,5-Tetrahydro-7,8-dihydroxy-1-phenyl- | |
| | 1H-3-benzazepine\|2-(Difluoromethyl)-5-{[(2-Methylpropanoyl)amino]methyl}-N-{5-Methyl-4-[4- | |
| | (Trifluoromethyl)phenyl]-1h-Imidazol-2-Yl}pyridine-3-Carboxamide\|3-[3-[4-(4-Chloro-phenyl)-6- | |
| | trifluoromethyl-pyrimidin-2-yl]-[1,2,4]oxadiazol-5-yl]-benzenesulfonamide\|Ladostigil\|2-[4-[2-[5- | |
| | (2,2-dimethylbutyl)-1H-imidazol-2-yl]ethyl]phenyl]benzoic acid\|4-Amino-3- | |
| | piperidinyl]ethyl]-1H-indole\|1-(2-Phenoxy-ethyl)-3-thiazol-2-yl-thiourea\|5,7-Dimethyl-3-thiophen- | |
| | 3-yl-quinoline\|(3S)-1-(3-chloropyridin-2-yl)-3-methyl-N-[4-(trifluoromethyl)phenyl]-3,6-dihydro-2H- | |
| | pyridine-4-carboxamide\|5-{3-[6-Trifluoromethyl-4-(4-trifluoromethyl-phenyl)-pyridin-2-yl]-phenyl}- | |
| | pyridin-2-ylamine\|2-(2-Amino-3-methoxyphenyl)-4H-1-benzopyran-4-one\|5-((2-(6-Amino-9H- | |
| | purin-9-yl)ethyl)amino)pentan-1-ol\|Selumetinib\|4-(3-(Ethylsulfonamido)-2-methylphenoxy)-2-((2- | |
| | fluoro-4-iodophenyl)amino)-1-methyl-5-fluoro-6-oxo-1,6-dihydropyridine-3-carboxamide | |
| HCK | Doramapimod\|1-cyclopentyl-3-(1H-pyrrolo[2,3-b]pyridin-5-yl)-1H-pyrazolo[3,4-d]pyrimidin-4- | Inhibitor |
| | amine\|N-(2-chloro-6-methylphenyl)-2-[(2,6-dimethylpyrimidin-4-yl)amino]-1,3-thiazole-5- | |
| | carboxamide\|N-(4-(((4-((((3-(1,1-Dimethylethyl)-1-(4-methylphenyl)-1H-pyrazol-5- | |
| | yl)amino)carbonyl)amino)-1-naphthalenyl)oxy)methyl)-2-pyridinyl)-2-methoxyacetamide\|3-(2-(3- | |
| | (Morpholinomethyl)phenyl)thieno[3,2-b]pyridin-7-ylamino)phenol\|3-(6-Allyloxy-2-naphthyl)-1-(4- | |
| | piperidylmethyl)pyrazolo[3,4-d]pyrimidin-4-amine\|3-(Naphthalen-1-Ylmethyl)-1-(Piperidin-4- | |
| | Ylmethyl)-1h-Pyrazolo[3,4-D]pyrimidin-4-Amine\|3-(6-Ethoxynaphthalen-2-Yl)-1-(Piperidin-4- | |
| | Ylmethyl)-1h-Pyrazolo[3,4-D]pyrimidin-4-Amine\|1-[4-(2-Anilinopyrimidin-4-yl)oxynaphthalen-1- | |
| | yl]-3-[5-tert-butyl-2-(4-methylphenyl)pyrazol-3-yl]urea\|N-(4-((4-(3-(3-(tert-Butyl)-1-(p-tolyl)-1H- | |
| | pyrazol-5-yl)ureido)naphthalen-1-yl)oxy)pyridin-2-yl)-2-methoxyacetamide\|1-[5-tert-butyl-2-(4- | |
| | methylphenyl)pyrazol-3-yl]-3-[4-[(1-methyl-2-oxo-3H-imidazo[4,5-b]pyridin-7-yl)oxy]naphthalen- | |
| | 1-yl]urea | |
| PKN3 | (4aR,5aR)-4,4a,5,5a-Tetrahydro-1H-cyclopropa(4,5)cyclopenta(1,2-C)pyrazole-3-carboxylic acid | Modulator |
| SPHK2 | 3-(4-chlorophenyl)-N-(pyridin-4-ylmethyl)adamantane-1-carboxamide\|(2R)-2-amino-2- | Modulator\| |
| | (methoxymethyl)-4-(4-octylphenyl)butan-1-ol\|(R)-2-amino-3-(4-octylphenylamino)propan-1- | Inhibitor |
| | ol\|(R)-2-amino-4-hydroxy-N-(4-octylphenyl)butanamide\|(S)-2-amino-4-hydroxy-N-(4- | |
| | octylphenyl)butanamide\|(S)-2-amino-6-hydroxy-N-(4-octylphenyl)hexanamide\|N,N- | |
| | dimethylsphingosine | |
| TXK | 3-[4-(5-Hydroxy-2-methylanilino)pyrimidin-2-yl]amino]benzenesulfonamide\|Blk-IN-1\|2-methyl-N- | Inhibitor |
| | [2-({3-[2-(prop-2-enamido) acetamido]phenyl}amino)pyrimidin-5-yl]-5-[3- | |
| | (trifluoromethyl)benzamido]benzamide | |
| AAK1 | 4-Piperidinemethanamine,N-(((2S)-7-chloro-2,3-dihydro-1,4-benzodioxin-2-yl)methyl)-1-(2- | Inhibitor |
| | methoxyphenyl)- | |
| M3K6 | 4-Tert-Butyl-N-[6-(1h-Imidazol-1-Yl)imidazo[1,2-A]pyridin-2-Yl]benzamide | Inhibitor |
| MP2K1 | Reversine\|Oroidin\|4,5-Dibromo-1h-pyrrole-2-carboxamide\|Debromohymenialdisine\|6,7- | Inhibitor |
| | dihydropyrrolo[2,3-c]azepine-4,8(1H,5H)-dione\|4,5,6,7-Tetrabromobenzotriazole\|5-Anilino-3-oxo- | |
| | 1,2-thiazole-4-carbonitrile\|2-(2-Amino-3-methoxyphenyl)-4H-1-benzopyran-4-one\|5-((2-(6- | |
| | Amino-9H-purin-9-yl)ethyl)amino)pentan-1-ol\|Selumetinib\|4-(3-(Ethylsulfonamido)-2- | |
| | methylphenoxy)-2-((2-fluoro-4-iodophenyl)amino)-1-methyl-5-fluoro-6-oxo-1,6-dihydropyridine-3- | |
| | carboxamide\|2-N-(4-piperazin-1-ylpyridin-3-yl)-4-N-pyridin-3-yl-1,3-thiazole-2,4-dicarboxamide | |
| MAPK3 | 2-(2-Quinolin-3-Ylpyridin-4-Yl)-1,5,6,7-Tetrahydro-4h-Pyrrolo[3,2-C]pyridin-4-One | Inhibitor |
| ACVL1 | 1-(4-(6-Methyl-5-(3,4,5-trimethoxyphenyl)pyridin-3-yl)phenyl)piperazine\|5-[6-(4- | Inhibitor\| |
| | Methoxyphenyl)pyrazolo[1,5-a]pyrimidin-3-yl]quinoline\|4-(4-(3-(1H-Pyrazol-4-yl)pyrazolo[1,5- | Modulator |
| | a]pyrimidin-6-yl)phenyl)morpholine\|4-[4-(3-Quinolin-4-ylpyrazolo[1,5-a]pyrimidin-6- | |
| | yl)phenyl]morpholine\|4-[6-[4-(4-Methylpiperazin-1-yl)phenyl]pyrazolo[1,5-a]pyrimidin-3- | |
| | yl]quinoline\|Robalzotan | |
| BIEA | Nadide\|Ursodeoxycholic acid | Inhibitor\| |
| | | Activator |
| PASK | 2-(4-amino-1-isopropyl-1H-pyrazolo[3,4-d]pyrimidin-3-yl)-1H-indol-5-ol | Inhibitor |
| ST38L | 2-[5-Chloro-2-(1-ethyl-5,5-dimethyl-2-oxo-2,3,4,5-tetrahydro-1H-benzo[b]azepin-8-ylamino)- | Inhibitor |
| | pyrimidin-4-ylamino]-3-fluoro-N-methyl-benzamide | |
| KSYK | Gusacitinib\|2-(2-pyrrolidin-1-ylethyl)-1H-indole\|Syk Inhibitor\|2-{[(1r,2s)-2- | Inhibitor\| |
| | Aminocyclohexyl]amino}-4-[(3-Methylphenyl)amino]pyrimidine-5-Carboxamide\|(2S)-N-(2,3- | Modulator |
| | dihydro-1H-inden-2-yl)-N-[(2-fluorophenyl)methyl]-2-[(1R)-1-naphthalen-1-yl-3,4-dihydro-1H- | |
| | isoquinolin-2-yl]propanamide\|Syk Inhibitor II\|3,4-Methylenedioxy-beta-nitrostyrene\|2-N-(3,5- | |
| | dichlorophenyl)-4-N-[4-(dimethylamino)cyclohexyl]-5-(3-methyl-1,2-oxazol-5-yl)pyrimidine-2,4- | |
| | diamine\|3-[4-(5-Hydroxy-2-methylanilino)pyrimidin-2-yl]amino]benzenesulfonamide\|N-Benzyl-4- | |
| | (2,5-dihydroxy-benzylamino)-benzamide\|Ellagic acid\|(R)-9-(4-(1-(dimethylamino)propan-2- | |
| | yl)phenyl)-8-hydroxy-6-methylthieno[2,3-c]quinolin-4(5H)-one\|2-[3-[4-[(2,2-difluoro-3-oxo-4H- | |
| | 1,4-benzoxazin-6-yl)amino]-5-fluoropyrimidin-2-yl]amino]phenoxy]-N- | |
| | methylacetamide\|Tamatinib\|2-[(1R,2S)-2-aminocyclohexyl]amino]-4-[3-(triazol-2- | |
| | yl)anilino]pyrimidine-5-carboxamide\|Pyrimethamine\|Urea,N-butyl-N′-(2-(3-(5-ethyl-4-phenyl-1H- | |
| | imidazol-1-yl)propoxy)-6-methylphenyl)-\|7-(2-(Methyloxy)-6-(((4-methyl-2- | |
| | pyridinyl)methyl)oxy)phenyl)-2,3,4,5-tetrahydro-1H-3-benzazepine\|1-(((Z)-(1-(2-aminothiazol-4- | |
| | yl)-2-oxo-2-(((3S,4R)-2-oxo-4-((2-oxooxazolidin-3-yl)methyl)-1-sulfoazetidin-3- | |
| | yl)amino)ethylidene)amino)oxy)cyclopropanecarboxylic acid\|1(2H)-Isoquinolinone,5,8-dichloro- | |
| | 2-((1,2-dihydro-4-methoxy-6-methyl-2-oxo-3-pyridinyl)methyl)-3,4-dihydro-7-((R)-methoxy-3- | |
| | oxetanylmethyl)-\|Mivavotinib\|Entospletinib\|Cerdulatinib\|Lanraplenib\|Fostamatinib disodium | |
| | anhydrous\|Fostamatinib\|1,4-Dihydroxyanthraquinone\|6H-(1,2,4) Triazolo(4,3- | |
| | a)(1,5)benzodiazepine-6-acetamide,4,5-dihydro-4-(1H-indol-3-ylmethyl)-N-(1-methylethyl)-5- | |
| | oxo-1-phenyl-N-(phenylmethyl)-,(S)-\|U.S. Pat No. 10,150,740, Example 9\|4-(3- | |
| | Nitrobenzylideneamino)Benzenesulfonamide | |
| PAK1 | 4-N-(5-cyclopropyl-1H-pyrazol-3-yl)-2-N-[(6-fluoro-1H-indazol-4-yl)methyl]-2-N-(2- | Inhibitor |
| | methoxyethyl)pyrimidine-2,4-diamine\|1-(3-Hydroxybenzyl)-3-(4-(pyridin-4-yl)thiazol-2- | |
| | yl)urea\|(2s)-1-{[6-Furan-3-Yl-5-(3-Methyl-2h-Indazol-5-Yl)pyridin-3-Yl]oxy}-3-(1h-Indol-3- | |
| | Yl)propan-2-Amine | |
| KKCC1 | 7-Oxo-7h-Benzimidazo[2,1-A]benz[de]isoquinoline-3-Carboxylic Acid | Inhibitor |
| DYRK2 | Harmine\|leucettine L41\|Dcdpc\|3-(2,7-Dimethoxyacridin-9-ylthio)propan-1-amine\|4,5,6,7- | Inhibitor |
| | Tetrabromo-N,N-dimethyl-1H-benzimidazol-2-amine\|Bicyclo(2.2.2)octane-1-carboxylic acid,4- | |
| | ((((3′R,4′S,5′R)-6″-chloro-4′-(3-chloro-2-fluorophenyl)-1′-ethyl-1″,2″-dihydro-2″- | |
| | oxodispiro(cyclohexane-1,2′-pyrrolidine-3′,3″-(3H)indol)-5′-yl)carbonyl)amino)- | |
| NEK7 | N-(7-chloro-1-oxo-1,2-dihydroisoquinolin-6-yl)-2-(cyclopropylamino)-2-phenylacetamide | Inhibitor |
| MK08 | 2-(2-chloro-4-iodophenylamino)-N-cyclopropylmethoxy-3,4-difluorobenzamide\|benzyl N-({2- | Inhibitor\| |
| | methyl-2-[(propan-2-yl)carbamoyl]cyclopropyl}(phenyl)methyl)carbamate\|JNK Inhibitor VIII\|Jnk- | Activator |
| | IN-8\|1,9-Pyrazoloanthrone\|Bisindolylmaleimide IV\|Bisindolylmaleimide i\|1,3-Benzothiazol-2-yl(2- | |
| | ((2-(3-pyridinyl)ethyl)amino)-4-pyrimidinyl)acetonitrile\|2-[(2-butoxypyrimidin-4-yl)amino]benzoic | |
| | Acid\|2-{[2-(Butylamino)pyrimidin-4-yl]amino}benzoic acid\|2-[(2-Phenoxypyrimidin-4- | |
| | yl)amino]benzoic acid\|2-[(2-Butan-2-yloxypyrimidin-4-yl)amino]benzoic acid\|2-{[2- | |
| | (Pentyloxy)pyrimidin-4-yl]amino}benzoic acid\|2-[(2-Propoxypyrimidin-4-yl)amino]benzoic acid\|2- | |
| | {[2-(Phenylamino)pyrimidin-4-yl]amino}benzamide\|(S)-4-(2-(N-methylisoquinoline-5- | |
| | sulfonamido)-3-oxo-3-(4-phenylpiperazin-1-yl)propyl)phenyl isoquinoline-5-sulfonate\|4,5,6,7- | |
| | Tetrabromobenzotriazole\|Acetamide,N-(4-amino-5-cyano-6-ethoxy-2-pyridinyl)-IN-(6- | |
| | ethoxypyridin-2-yl)acetamide\|N-(4-amino-5-cyano-6-phenylpyridin-2-yl)acetamide\|N-(4-amino-6- | |
| | butoxy-5-cyanopyridin-2-yl)acetamide\|bisindolylmaleimide IX\|1-tert-butyl-3-(naphthalen-1- | |
| | ylmethyl)-1H-pyrazolo[3,4-d]pyrimidin-4-amine\|Indiplon\|KT5720\|1-(((Z)-(1-(2-aminothiazol-4-yl)- | |
| | 2-oxo-2-(((3S,4R)-2-oxo-4-((2-oxooxazolidin-3-yl)methyl)-1-sulfoazetidin-3- | |
| | yl)amino)ethylidene)amino)oxy)cyclopropanecarboxylic acid\|sodium; 1H- | |
| | indazole; tetrachlororuthenium(1-)\|Azeliragon\|Disodium hydrogen phosphate\|Arbekacin\|CK1 | |
| | Inhibitor\|Diethyl 2,2,2-trifluoro-1-phenylethyl phosphate\|4-Anilino-2-(4-carbamoylpiperidin-1- | |
| | yl)pyrimidine-5-carboxamide\|Estradiol-17beta-glucuronide\|4-Anilino-2-[2- | |
| | (dimethylamino)ethylamino]pyrimidine-5-carboxamide\|4-Anilino-2-[(4-methylpyrimidin-2- | |
| | yl)amino]pyrimidine-5-carboxamide\|4-Anilino-2-(cyclohexylamino)pyrimidine-5- | |
| | carbonitrile\|Cefetamet Pivoxil HCl\|N,N-dimethyl-4-[3-(1H-pyrazol-4-yl)-1H-pyrrolo[2,3-b]pyridin-5- | |
| | yl]aniline\|N-(7-Chloro-4-oxo-1-phenyl-1,4-dihydro-quinolin-3-ylmethyl)-6-pyrrolidin-1-yl-nicotin- | |
| | amide\|Ac-Bth-Thr(PO3H2)-Pip-Nal-Gln-NH2\|5-cyclohexyl-3-(1-methylpyrazol-4-yl)-1H- | |
| | pyrrolo[2,3-b]pyridine\|N-[(1S,2R)-2-[(1,3-Benzothiazol-2-yl)amino]cyclopentyl]-2,6- | |
| | dimethoxybenzamide\|Metamizole\|2-(Cyclohexylamino)-8-(4-hydroxybutan-2-yl)pyrido[2,3- | |
| | d]pyrimidin-7-one\|Chlormezanone\|7-Chloro-3-[4-(2-hydroxyethylcarbamoyl)-benzyl]-4-oxo-1- | |
| | phenyl-1,4-dihydro-quinoline-2-carboxylic acid methyl ester\|4-Oxo-1-phenyl-3-[(4-sulfamoyl- | |
| | benzoylamino)-methyl]-7-trifluoromethyl-1,4-dihydro-[1,8]naphthyridine-2-carboxylic acid methyl | |
| | ester\|Thiolactomycin\|ethyl(7S)-11-oxo-14-(2-trimethylsilylethynyl)-2,4,10- | |
| | triazatetracyclo[10.4.0.02,6.07,10]hexadeca-1(16),3,5,12,14-pentaene-5-carboxylate\|3- | |
| | (thiophen-3-yl)-1H-indol-6-amine\|4,5-Dimethyl-2-[(1-naphthylacetyl)amino]-3- | |
| | thiophenecarboxamide\|n-(4-Chloro-3-(1h-1,2,4-triazol-5-yl)thiophen-2-yl)-2-(isoquinolin-5- | |
| | yl)acetamide\|North-methanocarbathymidine\|Methyl 3-[4-[5-(4-fluorophenyl)-2-pyrimidin-2- | |
| | ylpyrazol-3-yl]piperidin-1-yl]-2-phenylpropanoate\|3-Fluoro-5-(5-((2-methylthiazol-4- | |
| | yl)ethynyl)pyridin-2-yl)benzonitrile\|CID 6509858\|N-[(1S,2S)-2-[(6-Fluoro-1,3-benzothiazol-2- | |
| | yl)amino]cyclopentyl]-N-methyl-2-(1H-pyrazol-1-yl)benzamide\|Magnevist\|4-[(5-Piperazin-1- | |
| | ylpyridin-2-yl)amino]spiro[1,3,5,11-tetrazatricyclo[7.4.0.02,7]trideca-2,4,6,8-tetraene-13,1′- | |
| | cyclohexane]-10-one\|Pregnan-20-one,3-hydroxy-3-(trifluoromethyl)-,(3alpha,5alpha)-\|(1- | |
| | Methanesulfonyl-piperidin-4-yl)-{4-[4-(3-methyl-1h-pyrazol-4-yl)-benzotriazol-1-yl]-pyrimidin-2-yl}- | |
| | amine\|n-(4-Bromo-3-(1h-1,2,4-triazol-5-yl)thiophen-2-yl)-2-(2-oxo-6-(trifluoromethyl)quinolin- | |
| | 1(2h)-yl)acetamide\|2-(4-chlorophenyl)-6-phenyl-9H-purine\|Jnk-IN-5\|2-(2-(Benzo[b]thiophen-3- | |
| | yl)acetamido)thiophene-3-carboxamide\|3-[(E)-4-(dimethylamino)but-2-enoyl]amino]-N-[4-[4-(2- | |
| | phenylpyrazolo[1,5-a]pyridin-3-yl)pyrimidin-2-yl]amino]phenyl]benzamide\|1-Benzyl-2-oxo-1,2- | |
| | dihydro-pyridine-4-carboxylic acid (7-chloro-2-oxazol-2-yl-4-oxo-1-phenyl-1,4-dihydro-quinolin-3- | |
| | ylmethyl)-amide\|4-(2-(3-tert-Butylphenylamino)ethyl)-1H-imidazole | |
| KPCL | 1H-1,2,4-Triazole-1-propanamide,3-((2,2-difluoro-1,3-benzodioxol-5-yl)amino)-N,N-dimethyl-5- | .\|Inhibitor |
| | (4-pyridinyl)-\|(2S,3S,4R,5R)-2-(5-tert-Butyl-1,3,4-oxadiazol-2-yl)-5-(6-(4-chloro-2-fluoro- | |
| | anilino)purin-9-yl)tetrahydrofuran-3,4-diol\|2-Phenylthiomorpholine\|11-methyl-6,11-dihydro-5H- | |
| | benzo[a]carbazol-9-ol\|2,4,6-trimethyl-N-[4-(3-methylsulfonylphenyl)phenyl]methyl]-N-[5- | |
| | (trifluoromethyl)furan-2-yl]methyl]benzenesulfonamide\|1-Methoxy-3-(oxiran-2-ylmethoxy)-9H- | |
| | xanthen-9-one\|2-(3′-Methoxyphenyl)benzimidazole-4-carboxamide\|4- | |
| | [[(Hexylamino)carbonyl]amino]-N-[4-[2-[(2S)-2-hydroxy-3-(4- | |
| | hydroxyphenoxy)propyl]amino]ethyl]phenyl]-benzenesulfonamide\|Benzoic acid,((5alpha)-4,5- | |
| | epoxy-3,14-dihydroxy-17-(2-propenyl)morphinan-6-ylidene)hydrazide\|[2- | |
| | Phenylacetophenone]thiosemicarbazone\|Prostratin\|(18S)-18-(hydroxymethyl)-17-oxa-4,14,21- | |
| | triazahexacyclo[19.6.1.17,14.02,6.08,13.022,27]nonacosa-1(28),2(6),7(29),8,10,12,22,24,26- | |
| | nonaene-3,5-dione\|8-Octyl-benzolactam-V9\|Balanol | |
| CERK1 | N-(2-benzamido-1,3-benzothiazol-6-yl)adamantane-1-carboxamide | Inhibitor |
| KPCB | PKCbeta Inhibitor\|3-[1-[3-(Dimethylamino)propyl]-5-methoxy-1H-indol-3-yl]-4-(1H-indol-3-yl)-1H- | Inhibitor\| |
| | pyrrole-2,5-dione\|2-Aminoethyl dihydrogen phosphate\|3-(1H-Indol-3-yl)-4-phenylamino-pyrrole- | Modulator |
| | 2,5-dione\|Bisindolylmaleimide IV\|Prostratin\|(18S)-18-(hydroxymethyl)-17-oxa-4,14,21- | |
| | triazahexacyclo[19.6.1.17,14.02,6.08,13.022,27]nonacosa-1(28),2(6),7(29),8,10,12,22,24,26- | |
| | nonaene-3,5-dione\|Punicafolin\|BMS 813160-Bio-X\|Cercosporamide\|Phenol,4,4′-(3-methyl-1- | |
| | butenylidene)bis-\|(E)-3-(4-Hydroxyphenyl)-2,3-diphenylacrylonitrile\|4,4′- | |
| | (Cycloheptylidenemethylene)diphenol\|2-(4-Hydroxyphenyl)-3,3-diphenylacrylonitrile\|3,3-Bis(4- | |
| | methoxyphenyl)-2-phenylpropenenitrile\|2,3,3-Triphenylacrylonitrile\|3,3-Bis(4-hydroxyphenyl)-2- | |
| | phenylprop-2-enenitrile\|4-Cyclopentyliden(4-hydroxyphenyl)methylphenol\|Cyclofenil diphenol\|3- | |
| | (1-Methyl-1H-indol-3-yl)-4-[6,7,8,9-tetrahydro-12-methyl-7beta,8alpha- | |
| | (methaniminomethano)pyrido[1,2-a]indol]-10-yl]-1H-pyrrole-2,5-dione\|2,2′:5′,2″-Terthiophene-4- | |
| | carbaldehyde\|2,2′:5′,2″-Terthiophene-4,5″-dicarbaldehyde\|2,2′:5′,2″-Terthiophene-4- | |
| | methanol\|Linetastine\|Revumenib\|Sotrastaurin acetate\|Ruboxistaurin\|Enzastaurin\|Ruboxistaurin | |
| | Hydrochloride\|Balanol\|17-(Hydroxymethyl)-18-oxa-4,14,22- | |
| | triazahexacyclo[20.6.1.17,14.02,6.08,13.023,28]triaconta-1(29),2(6),7(30),8,10,12,23,25,27- | |
| | nonaene-3,5-dione\|(S)-3-(8-(Dimethylaminomethyl)-6,7,8,9-tetrahydropyrido(1,2-a)indol-10-yl)-4- | |
| | (1-methyl-3-indolyl)-1H-pyrrole-2,5-dione hydrochloride | |
| MET | U.S. Pat No. 10,09,2575, Example 101\|5-oxo-1-tosyl-2,5-dihydro-1H pyrrol-2-yl | CAR-T-Cell- |
| | acetate\|Trequinsin\|Sitravatinib\|Rusfertide\|(2S)-N-[(2R)-1-[2-[(2S)-1-[(2-amino-2- | Therapy(Dual |
| | oxoethyl) amino]-4-methyl-1-oxopentan-2-yl]amino]-2-oxoethyl]amino]-1-oxo-3-sulfanylpropan-2- | specific)\| |
| | yl]-2-[(2S,3R)-2-[(2S,3S)-2-[(2S)-2-[[(2R)-2-amino-3-sulfanylidenepropanoyl]amino]-3-(4- | CAR-T-Cell- |
| | hydroxyphenyl)propanoyl]amino]-3-methylpentanoyl]amino]-3- | Therapy\| |
| | hydroxybutanoyl]amino]butanediamide\|Acarbose\|Cbz-L-Phenylalaninal\|2- | Inhibitor\|.\| |
| | Aminoethenethiol\|methyl N-[(3R,4S,5S)-3-amino-1-[3-[3-amino-6-[2-fluoro-5-(propan-2- | Modulator\| |
| | ylcarbamoyl)phenyl]pyridine-2-carbonyl]amino]pyridin-4-yl]-5-methylpiperidin-4-yl]carbamate\|4- | Stimulator |
| | (1-Ethyl-7-piperazin-1-ylmethyl-1H-imidazo[4,5-c]pyridin-2-yl)-furazan-3-ylamine\|Farglitazar\|6,7- | |
| | Dimethoxy-3-(3-methoxy-phenyl)-quinoline\|4-Acetoxy-2-(4-methylphenyl)benzothiazole\|Pha- | |
| | 665752\|Ac-VTHRLAGLLSRSGGVVRKNFVPTDVGPFAF-NH2\|(2S)-1-(2-((2-((5-Cyano-2- | |
| | pyridyl)amino)ethyl)amino)acetyl)pyrrolidine-2-carbonitrile\|Cudc-101\|N-(quinolin-8-yl)-6- | |
| | (sulfamoylamino)hexanamide\|Met Kinase Inhibitor\|Ac-Ala-Gly-Ser-Cys(1)-Tyr-Cys(2)-Ser-Gly- | |
| | Pro-Pro-Arg-Phe-Glu-Cys(2)-Trp-Cys(1)-Tyr-Glu-Thr-Glu-Gly-Thr-Gly-Gly-Gly-Lys(Unk)-NH2\|N- | |
| | [(3R)-8-[[5-chloro-4-[2-fluoro-6-(methylcarbamoyl)anilino]pyrimidin-2-yl]amino]-5,5-dimethyl-2- | |
| | oxo-3,4-dihydro-1H-1-benzazepin-3-yl]pyrrolidine-1-carboxamide\|2-Benzyl-5-(3-Fluoro-4-{[6- | |
| | Methoxy-7-(3-Morpholin-4-Ylpropoxy)quinolin-4-Yl]oxy}phenyl)-3-Methylpyrimidin-4(3h)- | |
| | One\|Meclizine\|5-(1-Piperidin-4-ylpyrazol-4-yl)-3-(6-pyrrolidin-1-yl-1,3-benzoxazol-2-yl)pyridin-2- | |
| | amine\|2-[5-Chloro-2-(1-ethyl-5,5-dimethyl-2-oxo-2,3,4,5-tetrahydro-1H-benzo[b]azepin-8- | |
| | ylamino)-pyrimidin-4-ylamino]-3-fluoro-N-methyl-benzamide\|4-[(2S)-2-(3-Chlorophenyl)-2- | |
| | hydroxyethyl]amino]-3-[7-methyl-5-(4-morpholinyl)-1H-benzimidazol-2-yl]-2(1H)-pyridinone\|1- | |
| | (Phenylsulfonyl)-4-azaindole\|3-(phenylsulfonyl)-1H-pyrrolo[2,3-b]pyridine\|1-benzyl-1H- | |
| | pyrrolo[3,2-b]pyridine\|1-(2-nitrophenethyl)-1H-pyrrolo[3,2-b]pyridine\|Ethyl 5-benzoyl-4- | |
| | phenylthiazol-2-ylcarbamate\|1-(4-fluorophenyl)-N-[3-fluoro-4-[(3-phenyl-1H-pyrrolo[2,3-b]pyridin- | |
| | 4-yl)oxy]phenyl]-2,3-dimethyl-5-oxopyrazole-4-carboxamide\|Jnj-38877605\|MK-8033\|Sgx- | |
| | 523\|Etimizol\|Heptane-1,2,3-triol\|2-(4-(1-(quinolin-6-ylmethyl)-1H-[1,2,3]triazolo[4,5-b]pyrazin-6- | |
| | yl)-1H-pyrazol-1-yl)ethanol\|4-(2-(2-(3-(3-(1-Methyl-1hpyrazol-4-yl)-6-oxo-1,6-dihydropyridazin-1- | |
| | ylmethyl)-phenyl)-pyrimidin-5-yloxy)-ethyl)-morpholine)\|Golvatinib\|CID 9865731\|Altiratinib\|4- | |
| | Ethylpyrrolidine-2-imine\|2-Benzyloxycarbonylamino-3-{[(R)-1-(3-piperidin-4-yl-propionyl)- | |
| | piperidine-3-carbonyl]-amino}-propionic acid\|Csf1R-IN-2\|N-[4-(6,7-dimethoxyquinolin-4-yl)oxy-3- | |
| | fluorophenyl]-3-(4-fluorophenyl)-2,4-dioxo-1-propan-2-ylpyrimidine-5-carboxamide\|TP-271\|N-(3- | |
| | (9H-carbazol-9-yl)-2-hydroxypropyl)-N-(furan-2-ylmethyl)methanesulfonamide\|Zanzalintinib\|2′,3′- | |
| | Dideoxycytidine-5′-monophosphate\|MK-2461\|Aroplatin\|4-chloro-3-[(3-chloropyridin-2-yl)-(2- | |
| | hydroxyethyl)amino]-N-[3-(methylcarbamoyl)-4-(2-methylpropoxy)phenyl]benzamide\|2- | |
| | (Diphenylmethyl)-N-((2-methoxyphenyl)methyl)-1-azabicyclo(2.2.2)octan-3-amine\|1-(9-Methyl- | |
| | 9,15-diazatetracyclo[10.2.1.02,10.03,8]pentadeca-2(10),3(8),4,6-tetraen-6-yl)-4-[6- | |
| | (trifluoromethyl)pyridin-3-yl]pyridin-2-one\|Urea,N-[6-[6-(4-fluorophenyl)-1,2,4-triazolo[4,3- | |
| | b]pyridazin-3-yl]thio]-2-benzothiazolyl]-N′-[2-(4-morpholinyl)ethyl]-\|7-Methoxy-4-((6-phenyl- | |
| | [1,2,4]triazolo[4,3-b]pyridazin-3-yl)methoxy)quinoline\|Amg-337\|Merestinib\|Foretinib\|(R)-Lip-L- | |
| | glu-L-ala-OH\|1-(4-Amino-5-chloro-2-methoxyphenyl)-3-(1-butyl-4-piperidinyl)-1-propanone\|Ethyl | |
| | 3-[(4-chlorophenyl)methyl-[(5-nitrothiophen-2-yl)methyl]amino]methyl]pyrrolidine-1- | |
| | carboxylate\|Bozitinib\|Tivantinib\|(R/S)-2-((4-benzylphenoxy)methyl)piperazine\|N-[1-[1-[1-[2-[1- | |
| | [(2R)-1-(hydroxyamino)-4-methyl-1-oxopentan-2-yl]amino]-3-(1H-imidazol-5-yl)-1-oxopropan-2- | |
| | yl]amino]-2-oxoethyl]-2-oxoazepan-3-yl]amino]-1-oxopropan-2-yl]amino]-3-(1H-indol-3-yl)-1- | |
| | oxopropan-2-yl]-2-[3-(4- | |
| | hydroxyphenyl)propanoylamino]pentanediamide\|Savolitinib\|Capmatinib\|Tepotinib\|Crizotinib\| | |
| | Cabozantinib\|N-(4-((2-amino-3-chloropyridin-4-yl)oxy)-3-fluorophenyl)-4-ethoxy-1-(4-fluorophenyl)-2- | |
| | oxo-1,2-dihydropyridine-3-carboxamide | |
| BRD4 | Carbamic acid, (1-methyl-6-phenyl-4H-[1,2,4]triazolo[4,3-a][1,4]benzodiazepin-4-yl)-, | Inhibitor\| |
| | phenylmethyl ester (9CI)\|7-(3,5-Dimethylisoxazol-4-yl)-8-methoxy-1-((R)-1-(pyridin-2-yl)ethyl)- | Modulator |
| | 1H-imidazo[4,5-c]quinolin-2(3H)-one\|Methyl[(6s)-4-(4-Chlorophenyl)-2,3,9-Trimethyl-6h- | |
| | Thieno[3,2-F][1,2,4]triazolo[4,3-A][1,4]diazepin-6-Yl]acetate\|4-[(E)-(2-Amino-4-Hydroxy-5- | |
| | Methylphenyl)diazenyl]-N-(Pyridin-2-Yl)benzenesulfonamide\|2-[(6s)-4-(4-Chlorophenyl)-2,3,9- | |
| | Trimethyl-6h-[1,2]oxazolo[5,4-C]thieno[2,3-E]azepin-6-Yl]acetamide\|2-Methoxy-N-(3-Methyl-2- | |
| | Oxo-1,2,3,4-Tetrahydroquinazolin-6-Yl)benzenesulfonamide\|Colchiceine\|4-acetyl-N-[5- | |
| | (diethylsulfamoyl)-2-hydroxyphenyl]-3-ethyl-5-methyl-1H-pyrrole-2-carboxamide\|1,3-dimethyl-2- | |
| | oxo-2,3-dihydro-1H-benzimidazole-5-carboxamide\|6-(5-Bromo-2-Methoxyphenyl)-9h-Purin-2- | |
| | Amine\|[3-(3,5-Dimethyl-1,2-oxazol-4-yl)-5-[hydroxy(phenyl)methyl]phenyl] acetate\|(s)-2-(4-(4- | |
| | Chlorophenyl)-2,3,9-trimethyl-6h-thieno[3,2-f][1,2,4]triazolo[4,3-a][1,4]diazepin-6-yl)acetamide\|N- | |
| | (1,3-dimethyl-2-oxo-2,3-dihydro-1H-benzimidazol-5-yl)-2,4-dimethylbenzenesulfonamide\|N-(1,3- | |
| | dimethyl-2-oxo-6-piperidin-1-ylbenzimidazol-5-yl)-2-methoxybenzamide\|8-Chloro-1,4-Dimethyl- | |
| | 6-Phenyl-4h-[1,2,4]triazolo[4,3-A][1,3,4]benzotriazepine\|3-(dimethyl-1,2-oxazol-4-yl)-2-methoxy- | |
| | 9-[2-(trifluoromethoxy)phenyl]-7H,8H,9H-pyrrolo[2,3-c]1,5-naphthyridin-8-one\|9-benzyl-3- | |
| | (dimethyl-1,2-oxazol-4-yl)-2-methoxy-7H,8H,9H-pyrrolo[2,3-c]1,5-naphthyridin-8-one\|1-[3- | |
| | (Dimethyl-1,2-oxazol-4-yl)-5-ethoxyphenyl]ethan-1-ol\|(S)-(+)-tert-Butyl 2-(4-(4-chlorophenyl)- | |
| | 2,3,9-trimethyl-6H-thieno(3,2-f)(1,2,4)triazolo(4,3-a)(1,4)diazepin-6-yl)acetate\|2-[(4s)-6-(4- | |
| | Chlorophenyl)-8-Methoxy-1-Methyl-4h-[1,2,4]triazolo[4,3-A][1,4]benzodiazepin-4-Yl]-N- | |
| | Ethylacetamide\|6-Phenylethynyl-nicotinic acid methyl ester\|N-ethyl-4-[2-(4-fluoro-2,6- | |
| | dimethylphenoxy)-5-(2-hydroxypropan-2-yl)phenyl]-6-methyl-7-oxo-1H-pyrrolo[2,3-c]pyridine-2- | |
| | carboxamide\|(3~{r})-4-[2-[4-[1-(3-Methoxy-[1,2,4]triazolo[4,3-B]pyridazin-6-Yl)piperidin-4- | |
| | Yl]phenoxy]ethyl]-1,3-Dimethyl-Piperazin-2-One\|Birabresib\|2-[(2R)-2-amino-3- | |
| | phenylpropanoyl]amino]-N-[1-[1-[1-[2-[1-[(2R)-1-(hydroxyamino)-4-methyl-1-oxopentan-2- | |
| | yl]amino]-3-(1H-imidazol-5-yl)-1-oxopropan-2-yl]amino]-2-oxoethyl]-2-oxoazepan-3-yl]amino]-1- | |
| | oxopropan-2-yl]amino]-3-(1H-indol-3-yl)-1-oxopropan-2-yl]pentanediamide\|#N/A\|2-Amino-4- | |
| | furan-2-yl-6-phenyl-nicotinonitrile\|CPI-0610 anhydrous\|N-[4-chloro-3-[(2-methyl-3- | |
| | oxocyclopenten-1-yl)amino]phenyl]-2-methylbenzamide\|N-[4-chloro-3-[(2-methyl-3- | |
| | oxocyclopenten-1-yl)amino]phenyl]naphthalene-1-carboxamide\|N-[4-chloro-3-[(2-methyl-3- | |
| | oxocyclopenten-1-yl)amino]phenyl]piperidine-1-sulfonamide\|4-Hydroxycinnamic acid\|N-[4- | |
| | chloro-3-[(2-methyl-3-oxocyclopenten-1-yl)amino]phenyl]-1-phenylmethanesulfonamide\|N-[4- | |
| | chloro-3-[(2-methyl-3-oxocyclopenten-1-yl)amino]phenyl]-2- | |
| | methylbenzenesulfonamide\|U.S. Pat No. 9,550,796, Example 105\|U.S. Pat No., 955,0796, Example 111\|U.S. Pat No. | |
| | 9,550,796, Example 98\|U.S. Pat No. 9,550,796, Example 123\|U.S. Pat No. 9,550,796, Example 17\|Preparation of | |
| | (R)-(6-(4-chlorophenyl)-8-methoxy-1-methyl-4H-benzo[f][1,2,4]triazolo[4,3-a][1,4]diazepin-4-yl)methyl | |
| | cyclopropanesulfonate\|N-[3-hydroxy-5-[(R)-hydroxy(phenyl)methyl]phenyl]-N-methylprop-2- | |
| | enamide\|7-(2,3-Dihydro-1,4-benzodioxin-6-yl)-2-morpholin-4-ylthieno[2,3-e][1,3]oxazin-4-one\|N- | |
| | [3-hydroxy-5-[hydroxy(phenyl)methyl]phenyl]-N-methylprop-2-enamide\|Preparation of (R)-(6-(4- | |
| | chlorophenyl)-1-methyl-8-(1-methyl-1H-pyrazol-4-yl)-4H-benzo[f][1,2,4]triazolo[4,3- | |
| | a][1,4]diazepin-4-yl)methyl methanesulfonate\|Preparation of (R)-(6-(4-chlorophenyl)-8-methoxy- | |
| | 1-methyl-4H-benzo[f]-[1,2,4]triazolo[4,3-a][1,4]diazepin-4-yl)methyl methanesulfonate\|[(4R)-6-(4- | |
| | chlorophenyl)-8-[1-[2-(dimethylamino)-2-oxoethyl]pyrazol-4-yl]-1-methyl-4H-[1,2,4]triazolo[4,3- | |
| | a][1,4]benzodiazepin-4-yl]methyl methanesulfonate\|13-[2-Ethoxy-4-(3-pyrrolidin-1-ylpyrrolidine- | |
| | 1-carbonyl)anilino]-2,9-dimethyl-6-oxa-2,9,12-triazatricyclo[8.4.0.03,7]tetradeca- | |
| | 1(14),3(7),4,10,12-pentaen-8-one\|1-Phenyl-6-piperidin-1-ylpyrazolo[4,3-e][1,3]oxazin-4- | |
| | one\|Preparation (R)-(6-(4-chlorophenyl)-8-methoxy-1-methyl-4H-benzo[f][1,2,4]triazolo[4,3- | |
| | a][1,4]diazepin-4-yl)methyl ethanesulfonate\|1-phenyl-6-piperidin-1-yl-5H-pyrazolo[3,4- | |
| | d]pyrimidin-4-one\|2-[2-ethoxy-4-[(3S)-3-[(1-methylpiperidin-4-yl)amino]pyrrolidine-1- | |
| | carbonyl]anilino]-5,11-dimethylpyrido[4,3-b][1,4]benzodiazepin-6-one\|N-[3-hydroxy-5-[(S)- | |
| | hydroxy(phenyl)methyl]phenyl]-N-methylprop-2-enamide\|2-[2-Ethoxy-4-(4-pyrrolidin-1- | |
| | ylpiperidine-1-carbonyl)anilino]-5,11-dimethylpyrido[4,3-b][1,4]benzodiazepin-6-one\|6-(2- | |
| | Oxopiperidin-1-yl)-1-phenylpyrazolo[4,3-e][1,3]oxazin-4-one\|2-[2-Ethoxy-4-[4-(4-methyl-1,4- | |
| | diazepan-1-yl)piperidine-1-carbonyl]anilino]-5,11-dimethylpyrido[4,3-b][1,4]benzodiazepin-6- | |
| | one\|[(4R)-8-[1-(2-amino-2-oxoethyl)pyrazol-4-yl]-6-(4-chlorophenyl)-1-methyl-4H- | |
| | [1,2,4]triazolo[4,3-a][1,4]benzodiazepin-4-yl]methyl methanesulfonate\|[3- | |
| | [Hydroxy(phenyl)methyl]-5-[methyl(prop-2-enoyl)amino]phenyl]2,2- | |
| | dimethylpropanoate\|cyclopentyl(3S)-4-acetyl-7-(1-cyclobutylpyrazol-4-yl)-3-methyl-2,3- | |
| | dihydroquinoxaline-1-carboxylate\|cyclobutyl(3S)-4-acetyl-7-[1-(1,1-dioxo-1lambda6-thian-4-yl)- | |
| | 1H-pyrazol-4-yl]-3-methyl-1,2,3,4-tetrahydroquinoxaline-1-carboxylate\|(2-fluoro-2-methylpropyl) | |
| | (3S)-4-acetyl-3-methyl-7-[1-(oxetan-3-yl)pyrazol-4-yl]-2,3-dihydroquinoxaline-1-carboxylate | |
| PLK1 | #N/A\|N-[2-[2-(2-aminoethylamino)ethylamino]ethyl]-4-[(3R,5R,10S,13R,17R)-3-hydroxy-10,13- | Inhibitor\| |
| | dimethyl-2,3,4,5,6,7,8,9,11,12,14,15,16,17-tetradecahydro-1H-cyclopenta[a]phenanthren-17- | Modulator\|. |
| | yl]pentanamide\|2-[5-[3-(Dimethylamino)propyl]-2-methylpyridin-3-yl]amino]-9-(trifluoromethyl)- | |
| | 5,7-dihydropyrimido[5,4-d][1]benzazepine-6-thione\|4-[3,5-Bis(phenylmethoxy)phenyl]-2,4- | |
| | dioxobutanoic acid\|3-[(2-Bromophenyl)methoxy]-5-[6-(trifluoromethyl)benzimidazol-1- | |
| | yl]thiophene-2-carboxamide\|3-[(1R)-1-(2-chlorophenyl)ethoxy]-5-[6-(1-methylpiperidin-4- | |
| | yl)oxybenzimidazol-1-yl]thiophene-2-carboxamide\|1-[5-(5,6-dimethoxy-1H-1,3-benzodiazol-1-yl)- | |
| | 3-{[2-(trifluoromethyl)phenyl]methoxy}thiophen-2-yl]ethen-1-amine\|4-[(9-Cyclopentyl-7,7-difluoro- | |
| | 5-methyl-6-oxo-6,7,8,9-tetrahydro-5H-pyrimido[4,5-b][1,4]diazepin-2-yl)amino]-3-methoxy-N-(1- | |
| | methylpiperidin-4-yl)benzamide\|cyclohexane-1,2,3,4,5,6-hexol;(2R)-2-(3-fluoro-4- | |
| | phenylphenyl)propanoic acid\|2-[4-Acetamidylphenylamino]-4-[2-benzoylphenylamino]-5- | |
| | nitropyrimidine hydrochloride\|ZK-thiazolidinone\|Hmn-214\|(R)-5-(6-((4-methylpiperazin-1- | |
| | yl)methyl)-1H-benzo[d]imidazol-1-yl)-3-(1-(2-(trifluoromethyl)phenyl)ethoxy)thiophene-2- | |
| | carboxamide\|4-((9-Cyclopentyl-7,7-difluoro-5-methyl-6-oxo-6,7,8,9-tetrahydro-5H-pyrimido[4,5- | |
| | b][1,4]diazepin-2-yl)amino)-2-fluoro-5-methoxy-N-(1-methylpiperidin-4-yl)benzamide\|(1S)-1-(4- | |
| | (1S)-1-Aminoethyl)phenyl)-2-(tert-butylamino)ethanol\|(1S,3aR,6aS)-2-((R)-2-((S)-2-cyclohexyl- | |
| | 2-(pyrazine-2-carboxamido)acetamido)-3,3-dimethylbutanoyl)-N-((S)-1-(cyclopropylamino)-1,2- | |
| | dioxohexan-3-yl)octahydrocyclopenta[c]pyrrole-1-carboxamide\|Onvansertib\|4-{[(7r)-8- | |
| | Cyclopentyl-7-Ethyl-5-Methyl-6-Oxo-5,6,7,8-Tetrahydropteridin-2-Yl]amino}-3-Methoxy-N-(1- | |
| | Methylpiperidin-4-Yl)benzamide\|Volasertib\|Rigosertib\|2-cyclopentyl-4-(5-phenyl-1H-pyrrolo[2,3- | |
| | b]pyridin-3-yl)benzoic acid\|cyclo(-D-Me Tyr-L-Arg-L-Arg-L-Nal-Gly-)\|4-(2,6-Dimethylphenylthio)-2- | |
| | methoxybenzonitrile | |
| FGFR4 | Ascrolimus\|7-(4-fluoroanilino)-1-[(1R,3R)-3-hydroxycyclopentyl]-3-(4-methoxyphenyl)-4H- | .\|Inhibitor\| |
| | pyrimido[4,5-d]pyrimidin-2-one\|8-Bicyclo[2.2.1]hept-2-yl-2-{4-[4-(3-hydroxypropyl)piperidin-1- | Antagonist |
| | yl]phenylamino}-8H-pyrido[2,3-d]pyrimidin-7-one\|H3B- | |
| | 6527\|Fisogatinib\|Roblitinib\|Trimeprazine\|Actb-1003\|Des-AA1,2,5-[D-Trp8,IAmp9,Tyr11]Cbm- | |
| | SRIF | |
| KC1G1 | 2-[[3,4-Dioxo-2-(pyridin-4-ylamino)cyclobuten-1-yl]amino]-2-(3-hydroxyphenyl)acetamide | Inhibitor |
| IRAK4 | Triclofos sodium\|Sulfamic acid 3-(3-hydroxy-benzoyl)-phenyl ester\|4-Sulfamoylphenyl 4- | .\|Inhibitor |
| | guanidinobenzoate methanesulfonate\|Stanate (TN)\|5-(3-Carboxybenzoyl)-2-((6-(4- | |
| | methoxyphenyl)-5-hexenyl)oxy)benzenepropanoic acid\|Pegorgotein\|4- | |
| | Nitrobenzenesulfonamide\|6-Phenylethynyl-nicotinic acid methyl ester\|(2S)-2-amino-N-[(2S,13S)- | |
| | 2-[(3-tert-butyl-4-hydroxyphenyl)methyl]-3,7,14-trioxo-1,4,8-triazacyclotetradec-13-yl]-3- | |
| | phenylpropanamide\|N-(1H-benzo[d]imidazol-2-yl)-3-nitrobenzamide\|N-(1H-benzo[d]imidazol-2- | |
| | yl)-3-cyanobenzamide\|4-(4″-Fluoro-biphenyl-4-ylmethyl)pyridine\|2-[(3~{R})-12-(4-morpholin-4- | |
| | ylcyclohexyl)oxy-7-thia-9,11-diazatricyclo[6.4.0.0?{2,6}]dodeca-1(8),2(6),9,11-tetraen-3- | |
| | yl]ethanamide\|IRAK-1/4 Inhibitor\|N-[2-methoxy-4-(morpholin-4-yl)phenyl]-6-(1H-pyrazol-5- | |
| | yl)pyridine-2-carboxamide\|IRAK 4 inhibitor rac-45 [PMID: 18501603]\|(S)-2-Hydroxy-3-((R)-4- | |
| | ((trans-4-morpholinocyclohexyl)oxy)-6,7-dihydro-5H-cyclopenta[4,5]thieno[2,3-d]pyrimidin-5- | |
| | yl)propanamide\|2-[(2-aminoethyl)(methyl)amino]-N-(7-methoxyquinolin-6-yl)thieno[3,2- | |
| | d]pyrimidine-7-carboxamide\|N-{2-[4-(aminomethyl)piperidin-1-yl]-5-chlorophenyl}-6- | |
| | hydroxypyrazolo[1,5-a]pyrimidine-3-carboxamide\|6′-amino-N-(2-morpholinooxazolo[4,5- | |
| | b]pyridin-6-yl)-[2,3′-bipyridine]-6-carboxamide\|Tazobactam\|Oseni\|4-butyl-N-(7- | |
| | hydroxynaphthalen-1-yl)benzamide\|Indiplon\|(1R,3S)-3-[4-(2-methylheptan-2- | |
| | yl)phenyl]cyclohexan-1-ol\|MK-2206\|Dapivirine\|Urotensin-related peptide\|Bezafibrate\|\|- | |
| | Stepholidine\|N-[1-[5-[6-(1,3-benzothiazol-6-ylamino)-4-(propan-2-ylamino)pyridin-3-yl]-1,3,4- | |
| | thiadiazole-2-carbonyl]pyrrolidin-3-yl]acetamide\|2-methyl-1-(2-methyl-3-(trifluoromethyl)benzyl)- | |
| | 6-morpholino-1H-benzo[d]imidazole-4-carboxylic | |
| | acid\|Clebopride\|Perzinfotel\|Safinamide\|Calaxin\|Bedoradrine sulfate\|[[[[(2R,3S,4R,5R)-5-(6- | |
| | amino-2-propylsulfanylpurin-9-yl)-3,4-dihydroxyoxolan-2-yl]methoxy-hydroxyphosphoryl]oxy- | |
| | hydroxyphosphoryl]-difluoromethyl]phosphonic acid\|4-[4-(3-Quinolin-4-ylpyrazolo[1,5- | |
| | a]pyrimidin-6-yl)phenyl]morpholine\|3-[4-[5-(4-Fluorophenyl)-2-pyrimidin-2-ylpyrazol-3- | |
| | yl]piperidin-1-yl]-2-phenylpropanoic acid\|Unk-D-Val-D-Pro-D-Val-CF2CF3\|3-(6-Aminopyridin-3- | |
| | yl)-2-(1-cyclopentyl-1H-imidazol-4-yl)propionic acid | |
| BRD3 | Carbamic acid,(1-methyl-6-phenyl-4H-[1,2,4]triazolo[4,3-a][1,4]benzodiazepin-4-yl)-, | Inhibitor |
| | phenylmethyl ester (9CI)\|7-(3,5-Dimethylisoxazol-4-yl)-8-methoxy-1-((R)-1-(pyridin-2-yl)ethyl)- | |
| | 1H-imidazo[4,5-c]quinolin-2(3H)-one\|4-acetyl-N-[5-(diethylsulfamoyl)-2-hydroxyphenyl]-3-ethyl-5- | |
| | methyl-1H-pyrrole-2-carboxamide\|3-(dimethyl-1,2-oxazol-4-yl)-2-methoxy-9-[2- | |
| | (trifluoromethoxy)phenyl]-7H,8H,9H-pyrrolo[2,3-c]1,5-naphthyridin-8-one\|9-benzyl-3-(dimethyl- | |
| | 1,2-oxazol-4-yl)-2-methoxy-7H,8H,9H-pyrrolo[2,3-c]1,5-naphthyridin-8-one\|N-ethyl-4-[2-(4-fluoro- | |
| | 2,6-dimethylphenoxy)-5-(2-hydroxypropan-2-yl)phenyl]-6-methyl-7-oxo-1H-pyrrolo[2,3-c]pyridine- | |
| | 2-carboxamide\|U.S. Pat No. 9,55,0796, Example 17\|N-[3-hydroxy-5-[(R)-hydroxy(phenyl)methyl]phenyl]-N- | |
| | methylprop-2-enamide\|N-[3-hydroxy-5-[hydroxy(phenyl)methyl]phenyl]-N-methylprop-2- | |
| | enamide\|N-[3-hydroxy-5-[(S)-hydroxy(phenyl)methyl]phenyl]-N-methylprop-2-enamide\|[3- | |
| | [Hydroxy(phenyl)methyl]-5-[methyl(prop-2-enoyl)amino]phenyl] 2,2-dimethylpropanoate | |
| GLCNE | Ensartinib | Inhibitor |
| M3K19 | Senexin B | Inhibitor |
| DCLK1 | 1-(2,5-Dichlorobenzyl)-7-[6-(4-methylpiperazin-1-yl)pyridin-3-yl]-1,2,3,4-tetrahydropyrido[2,3- | Inhibitor |
| | b]pyrazine | |
| KS6B2 | 2-(2-((4-iodophenyl)amino)-2-oxoethoxy)-N-(2-(methyl(phenyl)amino)-2-oxoethyl)benzamide | Inhibitor |
| NEK1 | (2R,3R,4R)-1-[3-amino-6-(3,4,5-trimethoxyphenyl)pyrazin-2-yl]-2,3-dimethylpiperidine-4- | Inhibitor |
| | carboxylic acid | |
| CDKL2 | 3-(1H-indol-5-yl)-5-(4-((4-methylpiperazin-1-yl)methyl)phenyl)-1H-pyrrolo[2,3-b]pyridine | Inhibitor |
| MUSK | (S)-5-fluoro-2-(1-(5-fluoropyridin-2-yl)ethylamino)-6-(5-methyl-1H-pyrazol-3- | Inhibitor |
| | ylamino)nicotinonitrile\|7-Chloro-3-oxo-8-(1,3-thiazol-5-ylmethylamino)-11,16-dioxa-2,4,19,21- | |
| | tetrazatricyclo[15.3.1.05,10]henicosa-1(20),5,7,9,17(21),18-hexaene-18-carbonitrile | |
| CDK9 | Roniciclib\|Alvocidib\|(7S)-2-(2-aminopyrimidin-4-yl)-7-(2-fluoroethyl)-1,5,6,7-tetrahydro-4H- | Inhibitor\| |
| | pyrrolo[3,2-c]pyridin-4-one\|5-(2-aminopyrimidin-4-yl)-2-phenyl-1H-pyrrole-3- | Binder |
| | carboxamide\|Deschloroflavopiridol\|Meriolin 8\|Meriolin 6\|4-(4-Propoxy-1h-Pyrrolo[2,3-B]pyridin-3- | |
| | Yl)pyrimidin-2-Amine\|Meriolin 4\|4-(4-Methoxy-1h-Pyrrolo[2,3-B]pyridin-3-Yl)pyrimidin-2- | |
| | Amine\|Meriolin 2\|4-(1H-Pyrrolo[2,3-B]pyridin-3-YL)pyrimidin-2-amine\|Meriolin 7\|#N/A\|3,5- | |
| | Diamino-4-phenylazopyrazole\|4-({[6-(5-chloro-2-{[(1r,4r)-4-{[(2R)-1-methoxypropan-2- | |
| | yl]amino}cyclohexyl]amino}pyridin-4-yl)pyridin-2-yl]amino}methyl)oxane-4-carbonitrile\|ZK- | |
| | Cdk\|Dinaciclib\|Rgb-286638\|N-(5-(((5-(1,1-Dimethylethyl)-2-oxazolyl)methyl)thio)-2-thiazolyl)-4- | |
| | piperidinecarboxamide\|AZD4573 free base\|Fadraciclib\|7Mpp85iij3\|N-(1-((2- | |
| | (Dimethylamino)ethyl)amino)-2-methyl-1-oxopropan-2-yl)-4-(4-(2-methyl-5-((2S,3R,4R,5S,6R)- | |
| | 3,4,5-trihydroxy-6-(methylthio)tetrahydro-2H-pyran-2-yl)benzyl)phenyl)-butanamide\|Riviciclib | |
| | hydrochloride\|4-(2,4-difluorophenyl)-5-fluoro-N-[3-(methylsulfonylmethyl)-5-(pentafluoro- | |
| | lambda6-sulfanyl)phenyl]pyrimidin-2-amine\|3-[5-Fluoro-4-[4-methyl-2-(methylamino)-1,3-thiazol- | |
| | 5-yl]pyrimidin-2-l]amino]benzenesulfonamide\|(Rac)-[ethyl(3-{[5-fluoro-4-(4-fluoro-2- | |
| | methoxyphenyl)pyrimidin-2-yl]-amino}benzyl)oxido-lambda6-sulfanylidene]cyanamide\|3-[4-(2- | |
| | Methoxyphenyl)-2-pyridinyl]amino]-benzenemethanesulfonamide\|(2Z)-6-[(2,6- | |
| | dichlorophenyl)methylsulfonyl]-2-[(4-hydroxy-3-nitrophenyl)methylidene]-4H-1,4-benzothiazin-3- | |
| | one\|Tyr-Pro-Phe-Phe-OC(CH3)3\|2-[(3-Hydroxyphenyl)amino]-4-[4-Methyl-2-(Methylamino)-1,3- | |
| | Thiazol-5-Yl]pyrimidine-5-Carbonitrile\|[3-[4-(2,4-Difluorophenyl)-5-fluoropyrimidin-2-yl]amino]-5- | |
| | (pentafluoro-lambda6-sulfanyl)phenyl]methyl-methyl-oxo-lambda6-sulfanylidene]cyanamide\|6- | |
| | (4-fluoro-2-methoxyphenyl)-N-[4-[(methylsulfonimidoyl)methyl]-6-(trifluoromethyl)pyridin-2- | |
| | yl]pyrimidin-4-amine\|6-(4-fluoro-2-methoxyphenyl)-N-[4-(methylsulfonylmethyl)-6- | |
| | (trifluoromethyl)pyridin-2-yl]pyrimidin-4-amine\|U.S. Pat No. 9,670,161, 1 5-Fluoro-4-(4-fluoro-2- | |
| | methoxyphenyl)-N-{4-[(methylsulfonyl)methyl]pyridin-2-yl} pyrid,in-2-amine\|(rac)-4-{4-Fluoro-2-[(3- | |
| | fluorobenzyl)oxy]phenyl}-N-{3-[(S-methylsulfonimidoyl)methyl]phenyl}-1,3,5-triazin-2- | |
| | amine\|(rac)-5-Fluoro-4-(4-fluoro-2-methoxyphenyl)-N-{6-methoxy-4-[(S- | |
| | methylsulfonimidoyl)methyl]pyridin-2-yl}pyridin-2-amine\|5,6,7-Tribromo-2-piperazin-1-yl-1,3- | |
| | diazatricyclo[6.3.1.04,12]dodeca-2,4,6,8(12)-tetraene\|3-[(4-(2-Methoxyphenyl)-1,3,5-triazin-2- | |
| | yl)amino]benzene-methane-sulfonamide\|4-[4-[2-[5-[(5-tert-butyl-1,3-oxazol-2-yl)methylsulfanyl]- | |
| | 1,3-thiazol-2-yl]amino]-2-oxoethyl]phenoxy]-N-hydroxybutanamide\|2-N-(4-aminocyclohexyl)-9- | |
| | cyclopentyl-6-N-[6-(furan-2-yl)pyridin-3-yl]methyl]purine-2,6-diamine\|2-[(E)-2-(2,6- | |
| | dichlorophenyl)ethenyl]-5,7-dihydroxy-8-(3-hydroxy-1-methylpiperidin-4-yl)chromen-4- | |
| | one\|Piperidine-3-carboxylic acid[6-(2-methoxy-phenyl)-pyrimidin-4-yl]amide\|2-(4-Hydroxy- | |
| | phenyl)-7-phenyl-benzooxazol-5-ol\|N-[5-chloro-4-[3-(oxan-4-ylmethylamino)phenyl]pyridin-2- | |
| | yl]piperidine-3-carboxamide\|3-Methoxy-11-methyldibenz(b,f)oxazepine-8-carboxylate\|(S)-N2-{4- | |
| | [(3-chlorobenzyl)oxy]benzyl}serinamide\|2-(4-(bis(2-chloroethyl)amino)phenyl)-N-(5-((5-tert- | |
| | butyloxazol-2-yl)methylthio)thiazol-2-yl)acetamide\|Fascaplysin\|2-(2-Chlorophenyl)-2H-indazole- | |
| | 7-carboxamide | |
| KPCD | 13-Acetylphorbol\|Prostratin\|(18S)-18-(hydroxymethyl)-17-oxa-4,14,21- | Inhibitor\| |
| | triazahexacyclo[19.6.1.17,14.02,6.08,13.022,27]nonacosa-1(28),2(6),7(29),8,10,12,22,24,26- | Activator\|. |
| | nonaene-3,5-dione\|Bisindolylmaleimide IV[(1S,2R,6S,7S,8R,10S,11R,12R,14S,16S,17R,18R)- | |
| | 6,7-dihydroxy-8-(hydroxymethyl)-4,18-dimethyl-5-oxo-14-phenyl-16-prop-1-en-2-yl-9,13,15,19- | |
| | tetraoxahexacyclo[12.4.1.01,11.02,6.08,10.012,16]nonadec-3-en-17-yl](E)-3-phenylprop-2- | |
| | enoate\|Indolactam V\|Ingenol-3-bezoate\|Phenol,4,4′-(3-methyl-1-butenylidene)bis-\|(E)-3-(4- | |
| | Hydroxyphenyl)-2,3-diphenylacrylonitrile\|4,4′-(Cycloheptylidenemethylene)diphenol\|Phorbol | |
| | 12,13-dibutyrate\|2-(4-Hydroxyphenyl)-3,3-diphenylacrylonitrile\|3,3-Bis(4-methoxyphenyl)-2- | |
| | phenylpropenenitrile\|2,3,3-Triphenylacrylonitrile\|3,3-Bis(4-hydroxyphenyl)-2-phenylprop-2- | |
| | enenitrile\|Cyclofenil diphenol\|3-(1-Methyl-1H-indol-3-yl)-4-[6,7,8,9-tetrahydro-12-methyl- | |
| | 7beta,8alpha-(methaniminomethano)pyrido[1,2-a]indol]-10-yl]-1H-pyrrole-2,5-dione\|2,2′:5′,2″- | |
| | Terthiophene-4-carbaldehyde\|2,2′:5′,2″-Terthiophene-4,5″-dicarbaldehyde\|2,2′:5′,2″′- | |
| | Terthiophene-4-methanol\|Delcasertib\|[2-[[(2S,4S)-4-[(2R,4S,5R,6S)-4-(benzylamino)-5-hydroxy- | |
| | 6-methyloxan-2-yl]oxy-5,12-dihydroxy-7-methoxy-6,11-dioxo-1,2,3,4-tetrahydrotetracen-2- | |
| | yl]oxy]-2-oxoethyl]pentanoate\|17-(Hydroxymethyl)-18-oxa-4,14,22- | |
| | triazahexacyclo[20.6.1.17,14.02,6.08,13.023,28]triaconta-1(29),2(6),7(30),8,10,12,23,25,27- | |
| | nonaene-3,5-dione\|(S)-3-(8-(Dimethylaminomethyl)-6,7,8,9-tetrahydropyrido(1,2-a)indol-10-yl)-4- | |
| | (1-methyl-3-indolyl)-1H-pyrrole-2,5-dione hydrochloride\|3-[1-[3-(Dimethylamino)propyl]-5- | |
| | methoxy-1H-indol-3-yl]-4-(1H-indol-3-yl)-1H-pyrrole-2,5-dione\|3-Isobutyl-[1,4]thiazepan-(5E)- | |
| | ylideneamine\|3,4,5,6-Tetrahydrobenzo[c]quinolizin-3-(4aH)-one\|N(6)-Endonorbornan-2-yl-9- | |
| | methyladenine\|Bis-(5-pyridin-3-yl-thiophen-2-ylmethyl)-amine\|Bisindolylmaleimide i\|8-Octyl- | |
| | benzolactam-V9\|Dihexan-3-yl 5-(hydroxymethyl)isophthalate\|Diheptan-3-yl 5- | |
| | (hydroxymethyl)isophthalate\|Balanol | |
| M4K3 | 3-[(4-chlorophenyl)-(1H-imidazol-2-yl)methylidene]-5-[(1-ethylpiperidin-4-yl)amino]-1H-indol-2- | Inhibitor |
| | one | |
| CLK1 | (Z)-1-(3-Ethyl-5-methoxy-2,3-dihydrobenzothiazol-2-ylidene)propan-2-one\|Ethyl 3-[(E)-2-Amino- | Inhibitor |
| | 1-Cyanoethenyl]-6,7-Dichloro-1-Methyl-1h-Indole-2-Carboxylate\|leucettine L41\|5- | |
| | (benzo[d][1,3]dioxol-5-yl)-N-(3,5-dichlorobenzyl)pyrimidin-4-amine\|4-(1,3-benzodioxol-5-yl)-N- | |
| | [(3,5-dichlorophenyl)methyl]pyrimidin-2-amine | |
| M3K8 | Tpl2 Kinase Inhibitor\|MEK Inhibitor II\|7-vinyl-6H-chromeno[4,3-b]quinoline-3,9-diol\|Linolenic | Inhibitor\|. |
| | Acid\|4-Amino-1,8-naphthalimide\|Boc-Asp-Tyr(SO3H)-Nle-Unk-Trp-Nle-Asp-Phe-NH2\|4-[(7- | |
| | bromo-2-methyl-4-oxo-1H-quinazolin-6-yl)methyl-prop-2-ynylamino]-N-(pyridin-3- | |
| | ylmethyl)benzamide\|6-[4-(Piperidin-1-ylmethyl)phenyl]pyrrolo[2,3-b]pyridine-1-carboxamide\|8- | |
| | chloro-4-(3-chloro-4-fluoroanilino)-6-(1H-imidazol-5-ylmethylamino)-3-quinolinecarbonitrile | |
| AURKB | Deguelin\|N-(2-((1S,4R)-6-((4-(Cyclobutylamino)-5-(trifluoromethyl)pyrimidin-2-yl)amino)-1,2,3,4- | Inhibitor\| |
| | tetrahydro-1,4-epiminonaphthalen-9-yl)-2-oxoethyl)acetamide\|Sns-314\|MK-5108\|5-(3- | Modulator\|. |
| | (ethylsulfonyl)phenyl)-3,8-dimethyl-N-(1-methylpiperidin-4-yl)-9H-pyrido[2,3-b]indole-7- | |
| | carboxamide\|Cenisertib\|c[L-Tyr(OMe)-D-pro-L-Phe-D-trp]\|Domoic acid\|(Z)-3-(3-(((4- | |
| | ((dimethylamino)methyl)phenyl)amino)(phenyl)methylene)-2-oxoindolin-6-yl)-N- | |
| | ethylpropiolamide\|3-(4-(4-(2-(3-((dimethylamino)methyl)phenyl)-1H-pyrrolo[2,3-b]pyridin-4-yl)-1- | |
| | ethyl-1H-pyrazol-3-yl)phenyl)-1,1-dimethylurea\|Fluorometholone\|Azd-1152hqpa\|4-Methyl-5-(2- | |
| | (4-morpholinophenylamino)pyrimidin-4-yl)thiazol-2-amine\|N-(4-(3-(2-aminopyrimidin-4-yl)pyridin- | |
| | 2-yloxy)phenyl)-4-(4-methylthiophen-2-yl)phthalazin-1- | |
| | amine\|Sulfadiazine\|Danusertib\|Tozasertib\|Ilorasertib\|1-Cyclopropyl-3-(3-(5-(morpholinomethyl)- | |
| | 1H-benzo[d]imidazol-2-yl)-1H-pyrazol-4-yl)urea\|Orantinib\|N-[3-methoxy-4-(4-methylpiperazin-1- | |
| | yl)phenyl]-1-(4-methoxyphenyl)pyrazolo[3,4-b]pyridin-6-amine\|Aurora Kinase Inhibitor II\|3-((R)-1- | |
| | ((2S,6R)-2,6-dimethylmorpholino)ethyl)-N-(6-methyl-3-(1H-pyrazol-4-yl)imidazo[1,2-a]pyrazin-8- | |
| | yl)isothiazol-5-amine\|N-(3-fluorophenyl)-2-(2-(7-(3-(4-(hydroxymethyl)piperidin-1-yl)propoxy)-6- | |
| | methoxyquinazolin-4-ylamino)thiazol-5-yl)acetamide\|H-DL-Tyr-DL-Pro-Phe-D-Pro- | |
| | NH2\|Squalamine\|U.S. Pat No. 9,682,910, 4-(2-(benzo[d][1,3]dioxol-5-ylethyl)benzene-1,3-diol | |
| | (15)\|0\|Indirubin-3-methoxime\|Indirubin-3′-monoxime\|6-Bromoindirubin-3′-oxime\|Indirubin-3- | |
| | acetoxime\|#N/A\|7-Fluoroindirubin-3-acetoxime\|7-Bromoindirubin-3-oxime\|Hesperadin\|N3-(4- | |
| | fluorophenyl)-1h-pyrazolo[3,4-d]pyrimidine-3,4-diamine\|(3z)-N,N-Dimethyl-2-Oxo-3-(4,5,6,7- | |
| | Tetrahydro-1h-Indol-2-Ylmethylidene)-2,3-Dihydro-1h-Indole-5-Sulfonamide\|4-(1H-pyrazol-4-yl)- | |
| | 1H-pyrrolo[2,3-b]pyridine | |
| FGFR1 | Derazantinib\|[D-Ncy(isopropyl)7]acyline\|2-[3-(4-Phenylphenyl)propanamido]benzoic acid\|Actb- | Inhibitor\|.\| |
| | 1003\|4-(4-ethylpiperazin-1-yl)-N-[6-(2-fluoro-3-methoxyphenyl)-1H-indazol-3-yl]benzamide\|3-[(4- | Agonist\| |
| | chlorophenyl)-(1H-imidazol-2-yl)methylidene]-5-[(1-ethylpiperidin-4-yl)amino]-1H-indol-2-one\|N- | Modulator\| |
| | [(1S)-1-(5-fluoropyridin-2-yl)ethyl]-3-(3-propan-2-yloxy-1H-pyrazol-5-yl)imidazo[4,5-b]pyridin-5- | Binder\| |
| | amine\|3-(3-methoxybenzyl)-1H-pyrrolo[2,3-b]pyridine\|#N/A\|2-(Pyridin-4-Ylmethylamino)-~{n}-[3- | Antagonist |
| | (Trifluoromethyl)phenyl]benzamide\|6-(2,6-dichlorophenyl)-8-methyl-2-((3- | |
| | (methylthio)phenyl)amino)pyrido[2,3-d]pyrimidin-7(8H)-one\|3-(1H-Benzimidazol-2-YL)-1H- | |
| | indazole\|7-(4-fluoroanilino)-1-[(1R,3R)-3-hydroxycyclopentyl]-3-(4-methoxyphenyl)-4H- | |
| | pyrimido[4,5-d]pyrimidin-2-one\|6-(2,6-Dichlorophenyl)-2-{[3-(hydroxymethyl)phenyl]amino}-8- | |
| | methylpyrido[2,3-D]pyrimidin-7(8H)-one\|6-(2,6-Dichlorophenyl)-8-methyl-2-(4-morpholin-4- | |
| | ylanilino)pyrido[2,3-d]pyrimidin-7-one\|Pyrido(2,3-d)pyrimidin-7(8H)-one,6-(2,6-dichlorophenyl)- | |
| | 2-((4-fluorophenyl)amino)-8-methyl-\|2-[(4-Ethoxyphenyl)amino]-8-methyl-6-(2,6- | |
| | dichlorophenyl)pyrido[2,3-d]pyrimidine-7(8H)-one\|2-[4-[2- | |
| | [Ethyl(acetyl)amino]ethoxy]phenyl]amino]-8-methyl-6-(2,6-dichlorophenyl)pyrido[2,3- | |
| | d]pyrimidine-7(8H)-one\|6-(2,6-Dichlorophenyl)-2-[(4-Fluoro-3-Methylphenyl)amino]-8- | |
| | Methylpyrido[2,3-D]pyrimidin-7(8h)-One\|3-[5-[(Z)-[5-(anilinocarbamoyl)-2-oxo-1H-indol-3- | |
| | ylidene]methyl]-2,4-dimethyl-1H-pyrrol-3-yl]propanoic acid\|8-Bicyclo[2.2.1]hept-2-yl-2-{4-[4-(3- | |
| | hydroxypropyl)piperidin-1-yl]phenylamino}-8H-pyrido[2,3-d]pyrimidin-7-one\|2(1H)-Pyridinone,4- | |
| | ((2-(4-chloro-1H-pyrazol-1-yl)ethyl)amino)-3-(5-(1-(3-fluoropropyl)-4-piperidinyl)-7-methyl-1H- | |
| | benzimidazol-2-yl)-\|(E)-(4-(2-(1H-indazol-3-yl)vinyl)phenyl)(piperazin-1- | |
| | yl)methanone\|Martinostat\|MK-2461\|Debio-1347\|2-Amino-2-(3-chlorophenyl)acetic acid\|3-(3- | |
| | Cyclopentyloxy-4-methoxyphenyl)-3-(1,3-dioxoisoindol-2-yl)propanamide\|Valerenic acid | |
| | amide\|Azd-4547\|Infigratinib\|Sulfatinib\|Lucitanib\|Orantinib\|Pemigatinib\|Semaxanib\|Nintedanib | |
| KPCG | 3-[1-[3-(Dimethylamino)propyl]-5-methoxy-1H-indol-3-yl]-4-(1H-indol-3-yl)-1H-pyrrole-2,5- | Inhibitor |
| | dione\|Bisindolylmaleimide IV\|Prostratin\|Bisindolylmaleimide i\|(18S)-18-(hydroxymethyl)-17-oxa- | |
| | 4,14,21-triazahexacyclo[19.6.1.17,14.02,6.08,13.022,27]nonacosa- | |
| | 1(28),2(6),7(29),8,10,12,22,24,26-nonaene-3,5-dione\|8-Octyl-benzolactam-V9\|2-(1H-Indole-3- | |
| | yl)-3-phenylmaleimide\|1H-Pyrrole-2,5-dione,3,4-bis(4-methoxyphenyl)-\|3,4-diphenyl-1H-pyrrole- | |
| | 2,5-dione\|2-(4-Methoxyphenyl)-3-phenylmaleimide\|Cercosporamide\|Phenol,4,4′-(3-methyl-1- | |
| | butenylidene)bis-\|(E)-3-(4-Hydroxyphenyl)-2,3-diphenylacrylonitrile\|4,4′- | |
| | (Cycloheptylidenemethylene)diphenol\|2-(4-Hydroxyphenyl)-3,3-diphenylacrylonitrile\|3,3-Bis(4- | |
| | methoxyphenyl)-2-phenylpropenenitrile\|2,3,3-Triphenylacrylonitrile\|3,3-Bis(4-hydroxyphenyl)-2- | |
| | phenylprop-2-enenitrile\|4-Cyclopentyliden(4-hydroxyphenyl)methylphenol\|Cyclofenil diphenol\|3- | |
| | (1-Methyl-1H-indol-3-yl)-4-[6,7,8,9-tetrahydro-12-methyl-7beta,8alpha- | |
| | (methaniminomethano)pyrido[1,2-a]indol]-10-yl]-1H-pyrrole-2,5-dione\|2,2′:5′,2″-Terthiophene-4- | |
| | carbaldehyde\|2,2′:5′,2″-Terthiophene-4,5″-dicarbaldehyde\|1,2-Dioctanoylglycerol\|2,2′:5′,2″- | |
| | Terthiophene-4-methanol\|Midostaurin\|Balanol\|17-(Hydroxymethyl)-18-oxa-4,14,22- | |
| | triazahexacyclo[20.6.1.17,14.02,6.08,13.023,28]triaconta-1(29),2(6),7(30),8,10,12,23,25,27- | |
| | nonaene-3,5-dione\|(S)-3-(8-(Dimethylaminomethyl)-6,7,8,9-tetrahydropyrido(1,2-a)indol-10-yl)-4- | |
| | (1-methyl-3-indolyl)-1H-pyrrole-2,5-dione hydrochloride | |
| STK4 | N,N-dimethyl-2-(pyridin-3-yloxy)ethanamine | Inhibitor |
| AVR2A | PF-04418948 | Inhibitor |
| ACV1B | 4-[4-(3-Quinolin-4-ylpyrazolo[1,5-a]pyrimidin-6-yl)phenyl]morpholine\|4-[6-[4-(4-Methylpiperazin- | Inhibitor |
| | 1-yl)phenyl]pyrazolo[1,5-a]pyrimidin-3-yl]quinoline\|4-[6-(4-Piperidin-4-ylphenyl)pyrazolo[1,5- | |
| | a]pyrimidin-3-yl]quinoline\|4-(6-(6-(Piperazin-1-yl)pyridin-3-yl)pyrazolo[1,5-a]pyrimidin-3- | |
| | yl)quinoline\|4-[6-[6-[(2S,6R)-2,6-dimethylpiperazin-1-yl]pyridin-3-yl]pyrazolo[1,5-a]pyrimidin-3- | |
| | yl]quinoline\|Synthesis of (S)-4-(2-(5-chloro-2-fluorophenyl)-5-ethylpyridin-4-ylamino)-N-(2- | |
| | hydroxy propyl)nicotinamide\|Synthesis of 4-{[2-(5-chloro-2-fluorophenyl)-5-(pyrrolidin-1- | |
| | yl)pyridin-4-yl]amino}-N-(1,3-dihydroxypropan-2-yl)pyridine-3-carboxamide\|Synthesis of N-(2- | |
| | aminoethyl)-4-[2-(5-chloro-2-fluoro-phenyl)-5-isopropyl-4-pyridyl]amino]pyridine-3-carboxamide | |
| LTK | (2R)-2-[5-[6-amino-5-[(1R)-1-[5-fluoro-2-(triazol-2-yl)phenyl]ethoxy]pyridin-3-yl]-4-methyl-1,3- | Inhibitor |
| | thiazol-2-yl]propane-1,2-diol | |
| KPCD1 | (4R)-5-[(2S,3R)-4-amino-1-[(3S,6S,9R,15S,18R,21S,24S,30S,31R)-18-(4-aminobutyl)-9-(2- | Modulator\| |
| | amino-2-oxoethyl)-3-[(2S)-butan-2-yl]-15-[(R)-carboxy(methoxy)methyl]-21-(carboxymethyl)-6- | Inhibitor |
| | [(2R)-1-carboxypropan-2-yl]-24,28,31-trimethyl-2,5,8,11,14,17,20,23,26,29-decaoxo-1-oxa- | |
| | 4,7,10,13,16,19,22,25,28-nonazacyclohentriacont-30-yl]amino]-3-hydroxy-1,4-dioxobutan-2- | |
| | yl]amino]-4-[(2S)-2-(decanoylamino)-3-(1H-indol-3-yl)propanoyl]amino]-5-oxopentanoic | |
| | acid\|Bpkdi\|7-Hydroxy-2,3,4,5-tetrahydro-1H-benzofuro[2,3-c]azepin-1-one\|CRT0066101 | |
| | Dihydrochloride\|Go-6976\|Antibiotic K 252a | |
| IGF1R | Phosphoaminophosphonic acid-adenylate ester\|alpha-D-Mannose\|L-Fucose\|5-(3- | Inhibitor\| |
| | Phenylmethoxyphenyl)-7-[3-(pyrrolidin-1-ylmethyl)cyclobutyl]pyrrolo[2,3-d]pyrimidin-4- | Modulator\|.\| |
| | amine\|(1R,3S)-5-[2-[(1R,3aS,7aS)-1-[(2R,3S)-3-(2-hydroxyethyl)heptan-2-yl]-7a-methyl- | Antagonist |
| | 1,2,3,3a,4,5,6,7-octahydroinden-4-yl]ethyl]-4-methylcyclohex-4-ene-1,3-diol\|4-[(2S)-2-(3- | |
| | Chlorophenyl)-2-hydroxyethyl]amino]-3-[7-methyl-5-(4-morpholinyl)-1H-benzimidazol-2-yl]-2(1H)- | |
| | pyridinone\|4-((1H-indazol-6-ylamino)methyl)benzene-1,2-diol\|4-((Naphthalen-2- | |
| | ylamino)methyl)benzene-1,2-diol\|2(1H)-Pyridinone,4-((2-(4-chloro-1H-pyrazol-1-yl)ethyl)amino)- | |
| | 3-(5-(1-(3-fluoropropyl)-4-piperidinyl)-7-methyl-1H-benzimidazol-2-yl)-\|1-(4-chlorophenyl)-2-(2,4- | |
| | dichlorophenyl)-5-methylsulfonyl-N-(piperidin-1-yl)-1H-imidazole-4-carboxamide\|1,6-Bis(4″- | |
| | aminophenyl)-hexa-1,3,5-triene\|2-Thiophenecarboxamide,N-(5-((4-(2-hydroxyacetyl)-1- | |
| | piperazinyl)methyl)-2-((1E)-2-(1H-indazol-3-yl)ethenyl)phenyl)-3-methyl-,4- | |
| | methylbenzenesulfonate (1:1)\|Olomoucine\|Malic acid\|4-[(1S,9S,11S,12R,15S,16R,24R)-12- | |
| | hydroxy-12-[(E)-3-hydroxyprop-1-enyl]-11-methyl-21-oxo-5- | |
| | hexacyclo[14.7.1.01,19.03,8.09,24.011,15]tetracosa-3(8),4,6,19-tetraenyl]benzonitrile\|Nvp- | |
| | aew541\|4-[2-(5-amino-1H-pyrazol-4-yl)-4-chlorophenoxy]-5-chloro-2-fluoro-N-(1,3-thiazol-4- | |
| | yl)benzenesulfonamide\|#N/A\|Ceftobiprole medocaril free acid\|Voacangine\|2-[(E)-2-(3- | |
| | nitrophenyl)vinyl]quinoline\|Picropodophyllin\|2-Trityl-4,5-dihydro-1,3-oxazole\|Laromustine\|N-(4- | |
| | Chloro-3-((3-methyl-2-butenyl)oxy)phenyl)-2-methyl-3-furancarbothioamide\|3-Decylsulfinyl-1,1,1- | |
| | trifluoropropan-2-one\|2-(1H-Indole-3-yl)-3-phenylmaleimide\|Linsitinib\|Histamine | |
| | phosphate\|Aggrenox\|Limaprost\|1-(5-Chloro-2-methoxyphenyl)-3-(2-methylquinolin-4-yl)urea\|N2- | |
| | (4-(4-isopropylpiperazin-1-yl)-2-methoxyphenyl)-N4-(quinolin-5-yl)-7H-pyrrolo[2,3-d]pyrimidine- | |
| | 2,4-diamine\|2-((2-((1-(2-(Dimethylamino)acetyl)-5-methoxyindolin-6-yl)amino)-7H-pyrrolo[2,3- | |
| | d]pyrimidin-4-yl)amino)-6-fluoro-N-methylbenzamide\|Tyrphostin AG 1024\|N-(4-(4- | |
| | Aminopiperidin-1-yl)-2-methoxyphenyl)-5-chloro-4-(1H-indol-3-yl)pyrimidin-2-amine\|example 166 | |
| | [WO2014154727] | |
| NEK2 | 3-[(1R)-1-(2-chlorophenyl)ethoxy]-5-[6-(1-methylpiperidin-4-yl)oxybenzimidazol-1-yl]thiophene-2- | Inhibitor |
| | carboxamide\|(3Z)-5-(2-chloroacetyl)-3-(1H-pyrrol-2-ylmethylidene)-1H-indol-2-one\|(2R,3R,4R)-1- | |
| | [3-amino-6-(3,4,5-trimethoxyphenyl)pyrazin-2-yl]-2,3-dimethylpiperidine-4-carboxylic acid | |
| EPHB4 | Tesevatinib\|WT7488Bzd3\|1H-Imidazo[2,1-f]purine-2,4(3H,8H)-dione,7-(5-hydroxy-2- | Modulator\|.\| |
| | methylphenyl)-8-(2-methoxyphenyl)-1-methyl-\|(2S)-2-[(4R)-4- | Inhibitor |
| | [(3R,5R,8R,9S,10S,13R,14S,17R)-3-hydroxy-10,13-dimethyl-2,3,4,5,6,7,8,9,11,12,14,15,16,17- | |
| | tetradecahydro-1H-cyclopenta[a]phenanthren-17-yl]pentanoyl]amino]-3-(1H-indol-3-yl)propanoic | |
| | acid\|Unii-3mae0mxi6K | |
| TESK1 | 1-[5-[6-(2,6-Dimethyl-4-pentan-3-yloxyphenyl)-2-pyrazin-2-ylpyrimidin-4-yl]-1,3-thiazol-2-yl]-3- | Inhibitor |
| | methylurea\|1-Ethyl-3-[5-[6-(4-methoxy-2,6-dimethylphenyl)-2-pyrazin-2-ylpyrimidin-4-yl]-1,3- | |
| | thiazol-2-yl]urea\|N-[5-[6-(2,6-dichloro-4-methoxyphenyl)-2-pyrazin-2-ylpyrimidin-4-yl]-1,3-thiazol- | |
| | 2-yl]acetamide | |
| CDK1 | Kenpaullone\|Roniciclib\|Hymenialdisine\|(7S)-2-(2-aminopyrimidin-4-yl)-7-(2-fluoroethyl)-1,5,6,7- | Inhibitor\| |
| | tetrahydro-4H-pyrrolo[3,2-c]pyridin-4-one\|2-fluoro-4-(4-(1-isopropyl-2-methyl-1H-imidazol-5- | Binder |
| | yl)pyrimidin-2-ylamino)-N-methylbenzamide\|4-(7-butyl-5H-pyrrolo[2,3-b]pyrazin-6- | |
| | yl)phenol\|Alsterpaullone,2-Cyanoethyl\|aminopurvalanol A\|Bms-265246\|Cdk1 inhibitor \|Cdk1/2 | |
| | Inhibitor III\|1H-Pyrazolo[3,4-b]quinoxalin-3-amine\|Cvt-313\|4-(6-Cyclohexylmethoxy-9H-purin-2- | |
| | ylamino)-N,N-diethylbenzamide\|Cdk4 Inhibitor\|9H-Purine-2,6-diamine,N2-(2-aminocyclohexyl)- | |
| | N6-(3-chlorophenyl)-9-ethyl-,cis-\|purvalanol A\|Alsterpaullone\|2-[[6-(3-Amino-5-chloroanilino)-9- | |
| | propan-2-ylpurin-2-yl]amino]-3-methylbutan-1-ol\|Indirubin-3′-monoxime\|Indirubin-5- | |
| | sulphonate\|SU9516\|Microxine\|(2,6-Diaminopyridin-3-yl)(phenyl)methanone\|Bisindolylmaleimide | |
| | IV\|(2S)-1-[(2R)-2-acetamido-3-[2-[2-[2-[3-[4-(1H-indol-3-yl)-2,5-dioxopyrrol-3-yl]indol-1- | |
| | yl]ethoxy]ethoxy]ethylsulfanyl]propanoyl]-N-[(2S)-6-amino-1-[(2S)-6-amino-1-(methylamino)-1- | |
| | oxohexan-2-yl]amino]-1-oxohexan-2-yl]pyrrolidine-2-carboxamide\|Bisindolylmaleimide i\|1- | |
| | Azakenpaullone\|Thieno analogue of kenpaullone\|4-Phenyl-2-(phenylamino)pyrimidine-5- | |
| | carbonitrile\|2-(p-Toluidino)-4-phenylpyrimidine-5-carbonitrile\|4-({5-Amino-1-[(2,6- | |
| | Difluorophenyl)carbonyl]-1h-1,2,4-Triazol-3-Yl}amino)benzenesulfonamide\|2-(1H-Indole-3-yl)-3- | |
| | phenylmaleimide\|1H-Pyrrole-2,5-dione,3,4-bis(4-methoxyphenyl)-\|3,4-diphenyl-1H-pyrrole-2,5- | |
| | dione\|2-(4-Methoxyphenyl)-3-phenylmaleimide\|3,4-Dihydro-6-[4-[3- | |
| | (methylsulfonyl)phenyl]methyl]amino]-5-(trifluoromethyl)-2-pyrimidinyl]amino]-2(1H)- | |
| | quinolinone\|N-(4-methylphenyl)-4-quinolin-4-ylpyrimidin-2-amine\|N-(4-methylphenyl)-4-quinolin- | |
| | 3-ylpyrimidin-2-amine\|1-Propanol,3-[4-[2-[(3-chlorophenyl)amino]-4-pyrimidinyl]-2- | |
| | pyridiny]amino]-[(2S)-6-amino-2-[4-amino-3-[(2S)-2-amino-4- | |
| | phenylbutanoyl]amino]benzoyl]amino]hexanoic acid\|Purvalanol B\|(4-Amino-2-((1- | |
| | (methylsulfonyl)piperidin-4-yl)amino)pyrimidin-5-yl)(2,3-difluoro-6- | |
| | methoxyphenyl)methanone\|ZK-Cdk\|Dinaciclib\|6-((2-((4-(2,4-Dichlorophenyl)-5-(4-methyl-1H- | |
| | imidazol-2-yl)pyrimidin-2-yl)amino)ethyl)amino)nicotinonitrile\|Rgb-286638\|3- | |
| | Pyridinemethanamine,5-(3-(4,6-difluoro-1H-benzimidazol-2-yl)-1H-indazol-5-yl)-N-ethyl-4- | |
| | methyl-\|N-(6,6-dimethyl-5-(1-methylpiperidine-4-carbonyl)-1,4,5,6-tetrahydropyrrolo[3,4- | |
| | c]pyrazol-3-yl)-3-methylbutanamide\|Riviciclib hydrochloride\|Seliciclib\|1H-pyrrole-2,5-dione,3-(1- | |
| | methyl-1h-indol-3-yl)-4-(1-methyl-6-nitro-1h-indol-3-yl)-\|2-(2-chlorophenyl)-5,7-dihydroxy-8- | |
| | [(2R,3S)-2-(hydroxymethyl)-1-methylpyrrolidin-3-yl]chromen-4-one\|Olomoucine\|3-[5-Fluoro-4-[4- | |
| | methyl-2-(methylamino)-1,3-thiazol-5-yl]pyrimidin-2-yl]amino]benzenesulfonamide\|ethyl (7S)-11- | |
| | oxo-14-(2-trimethylsilylethynyl)-2,4,10-triazatetracyclo[10.4.0.02,6.07,10]hexadeca- | |
| | 1(16),3,5,12,14-pentaene-5-carboxylate\|8-Methyl-2-morpholin-4-yl-7-phenoxychromen-4- | |
| | one\|Tyr-Pro-Phe-Phe-OC(CH3)3\|7-(4-tert-butylphenyl)-2-[5-(3,8-diazabicyclo[3.2.1]octane-3- | |
| | carbonyl)pyridin-2-yl]amino]-N,N-dimethylpyrrolo[2,3-d]pyrimidine-6-carboxamide\|Cpzen- | |
| | 45\|5,6,7-Tribromo-2-piperazin-1-yl-1,3-diazatricyclo[6.3.1.04,12]dodeca-2,4,6,8(12)-tetraene\|7- | |
| | cyclopentyl-2-[5-(4-isopropyl-piperazin-1-yl)-pyridin-2-ylamino]-7H-pyrrolo[2,3-d]pyrimidine-6- | |
| | carboxylic acid dimethylamide\|4-[4-[2-[5-[(5-tert-butyl-1,3-oxazol-2-yl)methylsulfanyl]-1,3-thiazol- | |
| | 2-yl]amino]-2-oxoethyl]phenoxy]-N-hydroxybutanamide\|2-N-(4-aminocyclohexyl)-9-cyclopentyl- | |
| | 6-N-[[6-(furan-2-yl)pyridin-3-yl]methyl]purine-2,6-diamine\|Fascaplysin | |
| FGR | N-(2-chloro-6-methylphenyl)-2-[(2,6-dimethylpyrimidin-4-yl)amino]-1,3-thiazole-5- | Inhibitor |
| | carboxamide\|N-[(1S)-1-(5-fluoropyridin-2-yl)ethyl]-3-(3-propan-2-yloxy-1H-pyrazol-5- | |
| | yl)imidazo[4,5-b]pyridin-5-amine | |
| TGFR2 | 4-(2-((4-(2-(Pyridin-2-yl)-5,6-dihydro-4H-pyrrolo[1,2-b]pyrazol-3-yl)quinolin-7- | Inhibitor\|. |
| | yl)oxy)ethyl)morpholine\|4-(4-(3-(1H-Pyrazol-4-yl)pyrazolo[1,5-a]pyrimidin-6- | |
| | yl)phenyl)morpholine\|4-[4-(3-Quinolin-4-ylpyrazolo[1,5-a]pyrimidin-6-yl)phenyl]morpholine\|3-[6- | |
| | (2-pyridin-2-yl-5,6-dihydro-4H-pyrrolo[2,1-e]pyrazol-3-yl)benzimidazol-1-yl]propan-1- | |
| | ol\|Dodecyltrimethylammonium | |
| KS6A5 | 4-(1-Ethyl-7-piperazin-1-ylmethyl-1H-imidazo[4,5-c]pyridin-2-yl)-furazan-3-ylamine\|9-(6- | Inhibitor |
| | Aminopyridin-3-yl)-1-[3-(trifluoromethyl)phenyl]-4a,5,6,6a,7,8,9,10,10a,10b- | |
| | decahydropyrimido[5,4-c][1,5]naphthyridine-2,4-dione | |
| PIM1 | leucettine L41\|4-(2-Chloro-10h-Phenoxazin-10-Yl)-N,N-Diethylbutan-1-Amine\|Benzo[c]-2,6- | Inhibitor |
| | naphthyridin-5-amine,N-(2-chlorophenyl)-7-(1H-1,2,4-triazol-5-yl)-\|Spiro[piperidine-4,3′- | |
| | [3H]pyrido[3,4-b]indole]-4′-carbonitrile,7′,8′-dichloro-1′,2′,4′,9′-tetrahydro-9′-methyl-1′-oxo-\|(5Z)- | |
| | 5-[3-(trifluoromethyl)benzylidene]-1,3-thiazolidine-2,4-dione\|4-Phenyl-1-(1-phenylbutyl)piperidin- | |
| | 4-ol\|(E)-5-(3-Fluorostyryl)isatin\|(2Z)-6-[(2,6-dichlorophenyl)methylsulfonyl]-2-[(4-hydroxy-3- | |
| | nitrophenyl)methylidene]-4H-1,4-benzothiazin-3-one | |
| NTRK3 | 4-cyano-N-(5-(1-(2-(dimethylamino)acetyl)piperidin-4-yl)-2′,3′,4′,5′-tetrahydro-[1,1′-biphenyl]-2- | Inhibitor\| |
| | yl)-1H-imidazole-2-carboxamide\|(Z)-1-(3-((3-((1H-pyrrol-2-yl)methylene)-2-oxoindolin-6- | Modulator |
| | yl)amino)-4-methylphenyl)-3-(2-fluoro-5-(trifluoromethyl)phenyl)urea\|(S)-5-fluoro-2-(1-(5- | |
| | fluropyridin-2-yl)ethylamino)-6-(5-methyl-1H-pyrazol-3-ylamino)nicotinonitrile\|5-chloro-N2-(1-(5- | |
| | fluoropyridin-2-yl)ethyl)-N4-(5-isopropoxy-1H-pyrazol-3-yl)pyrimidine-2,4-diamine\|N-[(1S)-1-(5- | |
| | fluoropyridin-2-yl)ethyl]-3-(3-propan-2-yloxy-1H-pyrazol-5-yl)imidazo[4,5-b]pyridin-5-amine\|3- | |
| | benzyl-N-(2-morpholinoethyl)quinoxalin-2-amine\|5-[4-[2-(4-oxo-2H-1,3-benzoxazin-3- | |
| | yl)ethoxy]phenyl]methyl]-1,3-thiazolidine-2,4-dione\|(3S,4S,5S)-5-(Hydroxymethyl)piperidine-3,4- | |
| | diol\|2-[6-(4-Chlorophenoxy)hexyl]-1-cyano-3-pyridin-4-ylguanidine\|3-amino-6-methyl-N-pyridin-2- | |
| | ylpyrazine-2-carboxamide\|Aekkdegpyrmehfrwgsppkd\|Altiratinib\|MK- | |
| | 2461\|Entrectinib\|Larotrectinib\|N-(6-fluoro-3-pyridyl)-1-[4-[(5-isopropyl-1H-pyrazol-3-yl)amino]- | |
| | 6,7-dihydro-5H-cyclopenta[d]pyrimidin-2-yl]pyrrolidine-2-carboxamide\|4-[3-amino-4-(oxan-4- | |
| | yloxy)-1H-indazol-6-yl]-2-(4-cyclohexylpiperazin-1-yl)-N-methylbenzamide\|4-[3-amino-4-(1,1,1- | |
| | trifluoropropan-2-yloxy)-1H-indazol-6-yl]-2-(4-phenylpiperazin-1-yl)benzamide\|4-[3-amino-4- | |
| | (oxan-4-yloxy)-1H-indazol-6-yl]-2-(4-phenylpiperazin-1-yl)benzamide\|XX8E5WP7FS | |
| ROCK1 | Fasudil\|2-(2-chloro-4-iodophenylamino)-N-cyclopropylmethoxy-3,4-difluorobenzamide\|1-[(1S)-2- | Inhibitor\| |
| | hydroxy-1-phenylethyl]-3-[4-(4-pyridinyl)phenyl]urea\|2′-Monophosphoadenosine 5′- | Modulator |
| | diphosphoribose\|Nalfurafine hydrochloride\|Aprosulate Sodium\|5-[2-(Imidazol-1- | |
| | yl)ethyl]quinoline\|1-(3-Hydroxybenzyl)-3-(4-(pyridin-4-yl)thiazol-2-yl)urea\|N-(7-chloro-1-oxo-1,2- | |
| | dihydroisoquinolin-6-yl)-2-(cyclopropylamino)-2-phenylacetamide\|2-[3,4-Dioxo-2-(pyridin-4- | |
| | ylamino)cyclobuten-1-yl]amino]-2-(3-hydroxyphenyl)acetamide\|(3R)-6-methoxy-N-[2-[(1- | |
| | methylpiperidin-4-yl)methoxy]-4-(1H-pyrazol-4-yl)phenyl]-1,2,3,4-tetrahydroisoquinoline-3- | |
| | carboxamide\|Hydroxyfasudil\|N-[2-(4-amino-1,2,5-oxadiazol-3-yl)-1-ethylimidazo[5,4-d]pyridin-7- | |
| | yl]methyl]piperidin-4-amine\|7H-ADENINE,N-(p-FLUOROPHENYL)-\|Bisindolylmaleimide | |
| | i\|Bisindolylmaleimide IV\|(S)-4-(2-(N-methylisoquinoline-5-sulfonamido)-3-oxo-3-(4- | |
| | phenylpiperazin-1-yl)propyl)phenyl isoquinoline-5-sulfonate\|4,5,6,7- | |
| | Tetrabromobenzotriazole\|bisindolylmaleimide IX\|KT5720\|2-Cyclohexylacetic acidbiphenyl-3-yl | |
| | ester\|N-[3-[2-(4-amino-1,2,5-oxadiazol-3-yl)-1-ethylimidazo[4,5-c]pyridin-6-yl]oxyphenyl]-4-(2- | |
| | morpholin-4-ylethoxy)benzamide\|Srtcx1003\|6-(piperidin-4-yloxy)isoquinolin-1(2H)-one\|(S)-2- | |
| | Methyl-1-[(4-methyl-5-isoquinoline)sulfonyl]-homopiperazine\|Netarsudil\|N-cyclobutyl-2-[3-[4-(1H- | |
| | indazol-5-ylamino)-5,6-dimethylpyrimidin-2-yl]phenoxy]acetamide\|U.S. Pat No. 10,125,144, Example 18\|N- | |
| | (2-(3-(2-(isopropylamino)ethoxy)phenyl)pyrimidin-4-yl)-1H-indazol-5-amine\|1-(4- | |
| | Hydroxybenzyl)imidazole\|4-[2-Chloro-3-(trifluoromethyl)benzoyl]-1-(2,4-difluorophenyl)piperazin- | |
| | 2-one\|N-[2-(5-methoxy-1,3-dihydroisoindol-2-yl)pyrimidin-4-yl]-1H-indazol-5-amine\|5-[4-(2- | |
| | aminopropan-2-yl)-2,6-difluorobenzoyl]-2H-isoquinolin-1-one\|[4-(1-Aminoethyl)-2,6- | |
| | difluorophenyl]-isoquinolin-5-ylmethanone\|1-(1h-Indazol-5-Yl)-3-(2-Phenylethyl)urea | |
| CSK21 | d[Leu4,Dab8]VP\|Sodium 5-(3-ethynylphenylamino)pyrimido[4,5-c]quinoline-8-carboxylate\|N-[5- | Inhibitor |
| | [[3-cyano-7-(cyclopropylamino)pyrazolo[1,5-a]pyrimidin-5-yl]amino]-2- | |
| | methylphenyl]acetamide\|[4,5,6,7-Tetrabromo-2-(Dimethylamino)-1h-Benzimidazol-1-Yl]acetic | |
| | Acid\|Apigenin\|4,5,6,7-Tetrabromobenzotriazole\|#N/A\|Ellagic acid\|(2R)-2-[(2R)-2-[(2R)-2-[[(2R)- | |
| | 2-[(2R)-2-[[(2R)-2-[(2R)-2-[(2S)-2-[(2R)-2-[(2S,3S,4R)-5-(6-aminopurin-9-yl)-3,4- | |
| | dihydroxyoxolane-2-carbonyl]pyrrolidine-1-carbonyl]pyrrolidin-1-yl]propanoyl]amino]-5- | |
| | (diaminomethylideneamino)pentanoyl]amino]-5-(diaminomethylideneamino)pentanoyl]amino]-5- | |
| | (diaminomethylideneamino)pentanoyl]amino]-5-(diaminomethylideneamino)pentanoyl]amino]-5- | |
| | (diaminomethylideneamino)pentanoyl]amino]-5-(diaminomethylideneamino)pentanoic acid\|5,6,8- | |
| | Trichloro-4-oxo-1,4-dihydroquinoline-3-carboxylic acid\|7,8-dichloro-4-oxo-1H-quinoline-3- | |
| | carboxylic Acid\|4,5,6,7-Tetrabromobenzimidazole\|5,6-Dichloro-1-beta-D- | |
| | ribofuranosylbenzimidazole\|(5-Oxo-5,6-dihydro-indolo[1,2-A]quinazolin-7-YL)-acetic | |
| | acid\|Silmitasertib\|Balanol\|Emodin | |
| M3K2 | 1,2,3,4-Tetrahydroisoquinolin-7-ol\|1-(4-Ethylphenylsulfonyl)-4-phenylazepan-4-ol\|6-(2,5-Difluoro- | .\|Inhibitor |
| | 4-hydroxy-phenyl)-naphthalen-2-ol\|Disodium;(3S)-4-[2-[1-(4-fluorophenyl)-3-propan-2-ylindol-2- | |
| | yl]ethynyl-oxidophosphoryl]-3-hydroxybutanoate\|Moxidectin\|Arecoline\|Centanafadine\|7-{4-[4- | |
| | (2,3-Dichlorophenyl)-1,4-diazepan-1-yl]butoxy}-1,2,3,4-tetrahydroquinolin-2-one\|Glucagon | |
| | receptor antagonists-4\|(R)-N-((1S,2R)-2-hydroxy-2,3-dihydro-1H-inden-1-yl)-3-((2S,3S)-2- | |
| | hydroxy-3-((R)-2-(2-(2-methoxyphenyl)acetamido)-3-(methylthio)propanamido)-4- | |
| | phenylbutanoyl)-5,5-dimethylthiazolidine-4-carboxamide\|3-Morpholin-4-ylmethyl-5H- | |
| | phenanthridin-6-one\|(R)-N-(3-(5-(2-cyclopropylpyrimidin-5-yl)-1H-pyrrolo[2,3-b]pyridine-3- | |
| | carbonyl)-2,4-difluorophenyl)-3-fluoropyrrolidine-1-sulfonamide\|Copper;3-[18-(2-carboxyethyl)- | |
| | 7,12-diethyl-3,8,13,17,23-pentamethylporphyrin-21-id-2-yl]propanoic acid\|2-(2-chloro-4- | |
| | iodophenylamino)-N-cyclopropylmethoxy-3,4-difluorobenzamide\|3-(1H-indol-5-yl)-5-(4-((4- | |
| | methylpiperazin-1-yl)methyl)phenyl)-1H-pyrrolo[2,3-b]pyridine\|3,4-dihydro-2H-pyrrol-5- | |
| | amine\|Lobeglitazone\|1,4-Diamino-2,3-dicyano-1,4-bis(o-aminophenylmercapto)butadiene | |
| TGFR1 | 2-morpholino-6-(thiophen-3-yl)-4H-pyran-4-one\|1-Propyl-3-(3-trifluoromethyl-phenyl)- | Inhibitor\| |
| | pyrrolidine\|N-allyl-4-(3-(4-bromophenyl)benzo[d]isothiazol-6-yloxy)-N-methylbutan-1-amine\|2,4- | Modulator\| |
| | Imidazolidinedione,1-(((5-(4-chlorophenyl)-2-furanyl)methylene)amino)-3-(4-(4-methyl-1- | Antagonist\|. |
| | piperazinyl)butyl)-\|Methyl 2-((3,4-dihydro-3,4-dioxo-1-naphthalenyl)amino)benzoate\|2-(5-chloro- | |
| | 2-fluorophenyl)-N-(pyridin-4-yl)pteridin-4-amine\|1-(4-(6-Methyl-5-(3,4,5-trimethoxyphenyl)pyridin- | |
| | 3-yl)phenyl)piperazine\|4-(2-((4-(2-(Pyridin-2-yl)-5,6-dihydro-4H-pyrrolo[1,2-b]pyrazol-3- | |
| | yl)quinolin-7-yl)oxy)ethyl)morpholine\|4-[4-(3-Quinolin-4-ylpyrazolo[1,5-a]pyrimidin-6- | |
| | yl)phenyl]morpholine\|4-[6-[4-(4-Methylpiperazin-1-yl)phenyl]pyrazolo[1,5-a]pyrimidin-3- | |
| | yl]quinoline\|3-[6-(2-pyridin-2-yl-5,6-dihydro-4H-pyrrolo[2,1-e]pyrazol-3-yl)benzimidazol-1- | |
| | yl]propan-1-ol\|4-(3-Pyridin-2-YL-1H-pyrazol-4-YL)quinoline\|5″-(Quinolin-4-yl)-2,4″-bithiazol-2″- | |
| | amine\|4-Pyridin-2-yl-5-quinolin-2-yl-thiazol-2-ylamine\|4-Pyridin-2-yl-5-quinolin-4-yl-thiazol-2- | |
| | ylamine\|4-Pyridin-3-yl-5-quinolin-4-yl-thiazol-2-ylamine\|Benzonitrile,3-(((5-(6-methyl-2-pyridinyl)- | |
| | 4-(6-quinoxalinyl)-1H-imidazol-2-yl)methyl)amino)-\|4-(4-(3-(pyridin-2-yl)-1H-pyrazol-4-yl)pyridin- | |
| | 2-yl)-N-(tetrahydro-2H-pyran-4-yl)benzamide\|Benzamide,3-((4-(6-methyl-2-pyridinyl)-5-(6- | |
| | quinoxalinyl)-1H-imidazol-2-yl)methyl)-\|6-(2-(tert-Butyl)-5-(6-methylpyridin-2-yl)-1H-imidazol-4- | |
| | yl)quinoxaline\|2-(4-(benzo[d][1,3]dioxol-5-yl)-2-tert-butyl-1H-imidazol-5-yl)-6-methylpyridine\|2- | |
| | phenyl-N-(pyridin-4-yl)quinazolin-4-amine\|1-(1-Propionylpiperidin-4-yl)-3-p-tolylurea\|1- | |
| | (Adamantan-1-yl)-3-(1-propylpiperidin-4-yl)urea\|4-(4-(benzo[d][1,3]dioxol-5-yl)-5-(pyridin-2-yl)- | |
| | 1H-imidazol-2-yl)benzamide\|Potassium Iodide\|2-(4-((4-((1-cyclopropyl-3-(tetrahydro-2H-pyran-4- | |
| | yl)-1H-pyrazol-4-yl)oxy)pyridin-2-yl)amino)pyridin-2-yl)propan-2-ol\|Synthesis of 4-[2-(5-chloro-2- | |
| | fluoro-phenyl)-5-isopropyl-4-pyridyl]amino]-N-[2-hydroxy-1-(hydroxymethyl)ethyl]pyridine-3- | |
| | carboxamide\|Itacnosertib\|Granulatimide\|Vactosertib\|Galunisertib | |
| WNK3 | N-[(1S)-1-(5-fluoropyridin-2-yl)ethyl]-3-(3-propan-2-yloxy-1H-pyrazol-5-yl)imidazo[4,5-b]pyridin-5- | Inhibitor |
| | amine | |
| PGFRA | PDGF Receptor Tyrosine Kinase Inhibitor II\|1-cyclopentyl-3-(1H-pyrrolo[2,3-b]pyridin-5-yl)-1H- | Inhibitor\|.\| |
| | pyrazolo[3,4-d]pyrimidin-4-amine\|5-((5-(4-((4-(2-hydroxyethyl)piperazin-1-yl)methyl)-3- | Modulator |
| | (trifluoromethyl)phenylcarbamoyl)-2-methylphenyl)ethynyl)-N,1-dimethyl-1H-imidazole-2- | |
| | carboxamide\|3-[(4-chlorophenyl)-(1H-imidazol-2-yl)methylidene]-5-[(1-ethylpiperidin-4-yl)amino]- | |
| | 1H-indol-2-one\|1-(2-(5-(2-Methoxyethoxy)benzimidazol-1-yl)quinolin-8-yl)piperidin-4-ylamine\|4- | |
| | [[(2S)-2-(3-Chlorophenyl)-2-hydroxyethyl]amino]-3-[7-methyl-5-(4-morpholinyl)-1H-benzimidazol- | |
| | 2-yl]-2(1H)-pyridinone\|5,11-dimethyl-6H-pyrido[4,3-b]carbazol-9-ol\|N-(3-fluorophenyl)-6,7- | |
| | dimethoxy-1,4-dihydroindeno[1,2-c]pyrazol-3-amine\|5-[(Z)-(5-chloro-2-oxo-1,2-dihydro-3H-indol- | |
| | 3-ylidene)methyl]-N-[2-(diethylamino)ethyl]-2,4-dimethyl-1H-pyrrole-3-carboxamide\|6,7- | |
| | Dimethoxy-4-(3,4-dimethoxyphenoxy)quinoline\|6,7-dimethoxy-N-methyl-N-phenylquinazolin-4- | |
| | amine; hydrochloride\|6,7-Dimethoxy-3-pyridin-3-yl-quinoline\|4-(6,7-Dimethoxy-quinolin-3-yl)- | |
| | benzoic acid\|6,7-Dimethoxy-3-(4-methoxy-phenyl)-quinoline\|7-Fluoro-3-thiophen-3-yl- | |
| | quinoline\|6,7-Dimethoxy-3-thiophen-2-yl-quinoline\|7-Methoxy-3-thiophen-3-yl-quinoline\|5-Fluoro- | |
| | 3-thiophen-3-yl-quinoline\|3-(3,4-Dimethoxy-phenyl)-6,7-dimethoxy-quinoline\|4-(6,7- | |
| | Dimethoxyquinolin-3-yl)phenol\|7-Thiophen-3-yl-[1,3]dioxolo[4,5-g]quinoline\|6,7-Difluoro-3- | |
| | thiophen-3-yl-quinoline\|6,7-Dimethoxy-3-p-tolyl-quinoline\|6,7-Dimethoxy-3-(3-methoxy-phenyl)- | |
| | quinoline\|6,7-Dimethoxy-3-phenylethynyl-quinoline\|3-Cyclopentyl-6,7-dimethoxy-quinoline\|6- | |
| | Methoxy-3-thiophen-3-yl-quinoline\|3-(2-Cyclohexyl-ethyl)-6,7-dimethoxy-quinoline\|7-Methoxy-3- | |
| | pyridin-4-yl-quinoline\|6,7-Dimethoxy-3-((E)-styryl)-quinoline\|3-Pyridin-3-yl-quinoline-6,7- | |
| | diol\|Benzyl-(6,7-dimethoxy-quinolin-3-yl)-amine\|5,7-Dimethoxy-3-thiophen-3-yl-quinoline\|3- | |
| | Cyclopent-1-enyl-6,7-dimethoxy-quinoline\|3-(3-Fluoro-phenyl)-6,7-dimethoxy-quinoline\|3-(4- | |
| | Pyridinyl)quinoline\|5,7-Dimethyl-3-thiophen-3-yl-quinoline\|6,7-Dimethoxy-3-(2-methoxy-phenyl)- | |
| | quinoline\|3-Benzyloxy-6,7-dimethoxy-quinoline\|6,7-Dimethoxy-3-phenyl-quinoline\|6,7- | |
| | Dimethoxy-3-thiophen-3-yl-quinoline\|5-(6,7-Dimethoxy-quinolin-3-yl)-1H-pyridin-2-one\|3-(3,4- | |
| | Difluoro-phenyl)-6,7-dimethoxy-quinoline\|3-(4-Fluoro-phenyl)-6,7-dimethoxy-quinoline\|3-(3,4- | |
| | Dichloro-phenyl)-6,7-dimethoxy-quinoline\|3-(3-Thienyl)quinoline\|6-Methoxy-3-pyridin-4-yl- | |
| | quinoline\|7-Chloro-3-pyridin-4-yl-quinoline\|5,7-Dimethoxy-3-(4-pyridinyl)quinoline\|5,6,7- | |
| | Trimethoxy-3-pyridin-4-yl-quinoline\|3-((E)-Styryl)-quinoline\|3-(1H-indol-3-yl)-6,7-dimethoxy- | |
| | quinoline\|3-(1H-indol-3-yl)quinoline\|3-Cyclohexylethynyl-6,7-dimethoxy-quinoline\|6,7-Dichloro-3- | |
| | thiophen-3-yl-quinoline\|6,7-Dimethoxy-3-(4-nitro-phenyl)-quinoline\|3-Pyridin-4-yl-quinoline-5,7- | |
| | diol\|6,7-Dimethoxy-3-pyridin-4-yl-quinoline\|3-Pyridin-4-yl-quinolin-7-ol\|6-(2,6-dichlorophenyl)-8- | |
| | methyl-2-((3-(methylthio)phenyl)amino)pyrido[2,3-d]pyrimidin-7(8H)-one\|Flt-3 Inhibitor II\|(5- | |
| | fluoro-1H-indol-2-yl)-(1H-indol-2-yl)methanone\|Bis(1H-2-indolyl)methanone 12\|(1H-indol-2-yl)(5- | |
| | phenoxy-1H-indol-2-yl)methanone\|Bis(1H-2-indolyl)methanone 11\|(5-hydroxy-1H-indol-2-yl)-(1H- | |
| | indol-2-yl)methanone\|di(1H-indol-2-yl)methanone\|7-(4-fluoroanilino)-1-[(1R,3R)-3- | |
| | hydroxycyclopentyl]-3-(4-methoxyphenyl)-4H-pyrimido[4,5-d]pyrimidin-2-one\|6-(2,6- | |
| | Dichlorophenyl)-2-{[3-(hydroxymethyl)phenyl]amino}-8-methylpyrido[2,3-D]pyrimidin-7(8H)- | |
| | one\|6-(2,6-Dichlorophenyl)-8-methyl-2-(4-morpholin-4-ylanilino)pyrido[2,3-d]pyrimidin-7- | |
| | one\|Pyrido(2,3-d)pyrimidin-7(8H)-one,6-(2,6-dichlorophenyl)-2-((4-fluorophenyl)amino)-8- | |
| | methyl-\|2-[(4-Ethoxyphenyl)amino]-8-methyl-6-(2,6-dichlorophenyl)pyrido[2,3-d]pyrimidine- | |
| | 7(8H)-one\|2-[4-[2-[Ethyl(acetyl)amino]ethoxy]phenyl]amino]-8-methyl-6-(2,6- | |
| | dichlorophenyl)pyrido[2,3-d]pyrimidine-7(8H)-one\|6-(2,6-Dichlorophenyl)-2-[(4-Fluoro-3- | |
| | Methylphenyl)amino]-8-Methylpyrido[2,3-D]pyrimidin-7(8h)-One\|4-(1H-benzimidazol-1- | |
| | yl)aniline\|1H-Benzimidazole,5-methoxy-1-phenyl-\|1-Phenyl-5-hydroxybenzimidazole\|5- | |
| | Substituted 1-Phenylbenzimidazole 22\|1-Phenyl-1H-benzoimidazole\|3-benzyl-N-(2- | |
| | morpholinoethyl)quinoxalin-2-amine\|[2-(Octadecoxymethyl)oxolan-2-yl]methyl 2- | |
| | (trimethylazaniumyl)ethyl phosphate\|Amcasertib\|3-amino-6-methyl-N-pyridin-2-ylpyrazine-2- | |
| | carboxamide\|Amuvatinib\|5- | |
| | Methoxytryptoline\|Famitinib\|Crenolanib\|Lucitanib\|Avapritinib\|Ripretinib\|Semaxanib\|2- | |
| | (Aminomethyl)-5-(2″-naphthyl)tetrahydrofuran\|(Z)-3-(3,4-Dimethoxy-phenyl)-2-pyridin-3-yl- | |
| | acrylonitrile\|6,7-Dimethyl-2-phenylquinoxaline\|H-DL-Tyr(2,6-diMe)-DL-Tic-DL-Lys(Cbz)- | |
| | NHPh\|2,4″-Dimethoxy-5,3″-dipropyl-biphenyl\|N-[2-(3,4-dihydro-2H-quinolin-1-yl)-2-oxoethyl]-2- | |
| | [3-[(Z)-hydroxyiminomethyl]phenyl]carbamoylamino]-N-quinolin-8-ylacetamide\|Tinidazole\|2-(3- | |
| | Chlorophenyl)histamine | |
| PMVK | Isoferulic acid\|Cinnamic acid\|(3r)-3-Hydroxy-5-{[(R)-Hydroxy(Phosphonooxy)phosphoryl]oxy}-3- | Inhibitor |
| | Methylpentanoic Acid\|3-Hydroxy-3-methyl-6-phosphonohexanoic acid | |
| HIPK2 | N-[5-[3-cyano-7-(cyclopropylamino)pyrazolo[1,5-a]pyrimidin-5-yl]amino]-2- | Inhibitor |
| | methylphenyl]acetamide | |
| NUAK2 | N-(3-((5-chloro-2-((2-methoxy-4-(4-methylpiperazin-1-yl)phenyl)amino)pyrimidin-4- | Inhibitor |
| | yl)oxy)phenyl)propionamide | |
| CHKA | RSM-932A cation | Inhibitor |
| MK11 | 2-(2-chloro-4-iodophenylamino)-N-cyclopropylmethoxy-3,4- | Inhibitor |
| | difluorobenzamide\|Bisindolylmaleimide IV\|Bisindolylmaleimide i\|2-(4-Fluorophenyl)-4-methoxy-3- | |
| | (pyridin-4-YL)-1H-pyrrolo[2,3-B]pyridin-6-amine\|2-(Methylthio)-4-(4-fluorophenyl)-5-(4-pyridinyl)- | |
| | 1H-imidazole\|4-[5-(4-Fluorophenyl)-2-(4-methanesulfinyl-benzylsulfanyl)-3H-imidazol-4- | |
| | yl]pyridine\|p38 MAP Kinase Inhibitor III\|(S)-4-(2-(N-methylisoquinoline-5-sulfonamido)-3-oxo-3- | |
| | (4-phenylpiperazin-1-yl)propyl)phenyl isoquinoline-5-sulfonate\|4,5,6,7- | |
| | Tetrabromobenzotriazole\|2-chloro-5-(2-phenyl-5-(pyridin-4-yl)-1H-imidazol-4- | |
| | yl)phenol\|bisindolylmaleimide IX\|Indiplon\|KT5720\|4-(5-(4-Fluorophenyl)-3-(4-piperidyl)imidazol- | |
| | 4-yl)pyridine\|Neflamapimod | |
| MERTK | N-[4-(6,7-dimethoxyquinolin-4-yl)oxy-3-fluorophenyl]-4-ethoxy-1-(4-fluoro-2- | Inhibitor |
| | methylphenyl)pyrazole-3-carboxamide\|8-Bromo-2-((1-methylpiperidin-4-yl)amino)-4-((4- | |
| | phenoxyphenyl)amino)pyrido[4,3-D]pyrimidin-5(6H)-one\|1-[(Trans-4-Aminocyclohexyl)methyl]-N- | |
| | Butyl-3-(4-Fluorophenyl)-1h-Pyrazolo[3,4-D]pyrimidin-6-Amine\|(1r,4r)-4-(2-(butylamino)-5-(4-((4- | |
| | methylpiperazin-1-yl)methyl)phenyl)-7H-pyrrolo[2,3-d]pyrimidin-7- | |
| | yl)cyclohexanol\|cyclo[Cys6,Cys 10]N/OFQ(1-13) NH2\|N-[4-(6,7-dimethoxyquinolin-4-yl)oxy-3- | |
| | fluorophenyl]-3-(4-fluorophenyl)-2,4-dioxo-1-propan-2-ylpyrimidine-5- | |
| | carboxamide\|2MT30Ehi63\|(1s,2s)-2-(3,4-Dichlorobenzoyl)cyclopropanecarboxylic Acid\|(2S)-2- | |
| | [[(2S)-2-[(2S)-2-[(2S,3R)-2-[(2S)-2-[(2S)-2-[(2S)-2-[(2S)-2-[(2S)-2-[(2- | |
| | acetamidoacetyl) amino]propanoyl]amino]-5-amino-5-oxopentanoyl]amino]-3- | |
| | phenylpropanoyl]amino]-3-hydroxypropanoyl]amino]-6-aminohexanoyl]amino]-3- | |
| | hydroxybutanoyl]amino]propanoyl]amino]propanoyl]amino]-6-aminohexanoic acid\|MK-2461 \|N- | |
| | (5-((6,7-Dimethoxyquinolin-4-yl)oxy)pyridin-2-yl)-2,5-dioxo-1-phenyl-1,2,5,6,7,8- | |
| | hexahydroquinoline-3-carboxamide | |
| TIF1A | 3,4-Dimethoxy-N-[6-(4-Methoxyphenoxy)-1,3-Dimethyl-2-Oxo-2,3-Dihydro-1h-Benzimidazol-5- | Inhibitor |
| | Yl]benzenesulfonamide | |
| EPHB3 | 1H-Imidazo[2,1-f]purine-2,4(3H,8H)-dione,7-(5-hydroxy-2-methylphenyl)-8-(2-methoxyphenyl)- | Inhibitor |
| | 1-methyl-\|(2S)-2-[[(4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid | |
| ALK | 1H-Indole-2-carboxamide,5-chloro-3-((4-((2,4-diamino-5-pyrimidinyl)methyl)-6,7-dimethoxy-2- | Inhibitor\|.\| |
| | benzofuranyl)methyl)-N,N-dimethyl-\|(1S,2S,3R,4R)-3-((5-chloro-2-(((S)-1-methoxy-7- | Modulator |
| | morpholino-6,7,8,9-tetrahydro-5H-benzo[7]annulen-2-yl)amino)pyrimidin-4- | |
| | yl)amino)bicyclo[2.2.1]hept-5-ene-2-carboxamide.\|2-methyl-3-(pyridin-4-yl)-1H- | |
| | indole\|Droxidopa\|2-((2-((1-(2-(Dimethylamino)acetyl)-5-methoxyindolin-6-yl)amino)-7H- | |
| | pyrrolo[2,3-d]pyrimidin-4-yl)amino)-6-fluoro-N-methylbenzamide\|1-(2,5-Dichlorobenzyl)-7-[6-(4- | |
| | methylpiperazin-1-yl)pyridin-3-yl]-1,2,3,4-tetrahydropyrido[2,3-b]pyrazine\|[4-[1-[5-chloro-2- | |
| | (trifluoromethyl)phenyl]methyl]-3,4-dihydro-2H-pyrido[2,3-b]pyrazin-7-yl]phenyl]-(4-pyrrolidin-1- | |
| | ylpiperidin-1-yl)methanone\|1-[5-chloro-2-(trifluoromethyl)phenyl]methyl]-7-(2-piperazin-1- | |
| | ylpyridin-4-yl)-3,4-dihydro-2H-pyrido[2,3-b]pyrazine\|(2R)-2-[5-[6-amino-5-[(1R)-1-[5-fluoro-2- | |
| | (triazol-2-yl)phenyl]ethoxy]pyridin-3-yl]-4-methyl-1,3-thiazol-2-yl]propane-1,2-diol\|(1S,2S,3R,4R)- | |
| | 3-[5-Chloro-2-(1-ethyl-6-methoxy-2-oxo-2,3,4,5-tetrahydro-1H-benzo[b]azepin-7-ylamino)- | |
| | pyrimidin-4-ylamino]-bicyclo[2.2.1]hept-5-ene-2-carboxylic acid amide\|N-(4-(4-Aminopiperidin-1- | |
| | yl)-2-methoxyphenyl)-5-chloro-4-(1H-indol-3-yl)pyrimidin-2-amine\|Lorlatinib\|Belizatinib\|N2-[2- | |
| | Methoxy-4-[4-(4-methyl-1-piperazinyl)-1-piperidinyl]phenyl]-N4-[2-[(1- | |
| | methylethyl)sulfonyl]phenyl]-1,3,5-triazine-2,4-diamine\|(R)-6-Amino-5-(1-(2,6-dichloro-3- | |
| | fluorophenyl)ethoxy)-N-(4-(4-methylpiperazine-1-carbonyl)phenyl)pyridazine-3- | |
| | carboxamide\|Repotrectinib\|(2-((5-Chloro-2-((4-(4-(dimethylamino)piperidin-1-yl)-2- | |
| | methoxyphenyl)amino)pyrimidin-4-yl)amino)phenyl)dimethylphosphine | |
| | oxide\|Ensartinib\|Entrectinib\|Alectinib\|Crizotinib\|Ceritinib\|Brigatinib\|Bucladesine sodium\|Axitinib | |
| EPHA7 | 1H-Imidazo[2,1-f]purine-2,4(3H,8H)-dione,7-(5-hydroxy-2-methylphenyl)-8-(2-methoxyphenyl)- | Inhibitor |
| | 1-methyl-\|5-((5-(4-((4-(2-hydroxyethyl)piperazin-1-yl)methyl)-3- | |
| | (trifluoromethyl)phenylcarbamoyl)-2-methylphenyl)ethynyl)-N,1-dimethyl-1H-imidazole-2- | |
| | carboxamide\|(2S)-2-[(4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid | |
| ARBK2 | 3-({[4-Methyl-5-(Pyridin-4-Yl)-4h-1,2,4-Triazol-3-Yl]methyl}amino)-N-[2- | Inhibitor |
| | (Trifluoromethyl)benzyl]benzamide | |
| EPHA2 | 1H-Imidazo[2,1-f]purine-2,4(3H,8H)-dione,7-(5-hydroxy-2-methylphenyl)-8-(2-methoxyphenyl)- | Inhibitor\|.\| |
| | 1-methyl-\|5-((5-(4-((4-(2-hydroxyethyl)piperazin-1-yl)methyl)-3- | CAR-T-Cell- |
| | (trifluoromethyl)phenylcarbamoyl)-2-methylphenyl)ethynyl)-N,1-dimethyl-1H-imidazole-2- | Therapy |
| | carboxamide\|(2S)-2-[[4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid\|Phosphoaminophosphonic acid-adenylate | |
| | ester\|Bromosulfophthalein\|leukotriene D4\|Iotrolan\|4-Isopropyl-2- | |
| | (phenoxymethyl)morpholine\|Ziftomenib | |
| SLK | 6-Cyclopropyl-8-fluoro-2-[2-(hydroxymethyl)-3-[1-methyl-5-[5-(4-methylpiperazin-1-yl)pyridin-2- | Inhibitor |
| | yl]amino]-6-oxopyridin-3-yl]phenyl]isoquinolin-1-one | |
| KIT | Tertatolol\|3-[5-(3-Nitrophenyl)thiophen-2-yl]propanoic acid\|3-Phenyl-1H-benzofuro[3,2- | Inhibitor\| |
| | c]pyrazole\|4-cyano-N-(5-(1-(2-(dimethylamino)acetyl)piperidin-4-yl)-2′,3′,4′,5′-tetrahydro-[1,1′- | CAR-T-Cell- |
| | biphenyl]-2-yl)-1H-imidazole-2-carboxamide\|1-(4-((6,7-Dimethoxyquinolin-4-yl)oxy)-2- | Therapy\| |
| | methoxyphenyl)-3-(1-(thiazol-2-yl)ethyl)urea\|N-(3-chlorophenyl)-4-methyl-3-(2-pyrazolo[1,5- | Modulator |
| | a]pyrimidin-6-ylethynyl)benzamide\|3-[(4-chlorophenyl)-(1H-imidazol-2-yl)methylidene]-5-[(1- | |
| | ethylpiperidin-4-yl)amino]-1H-indol-2-one\|1-(2-(5-(2-Methoxyethoxy)benzimidazol-1-yl)quinolin- | |
| | 8-yl)piperidin-4-ylamine\|1-(4-((4-Ethylpiperazin-1-yl)methyl)-3-(trifluoromethyl)phenyl)-3-(4-((6- | |
| | (methylamino)pyrimidin-4-yl)oxy)phenyl)urea\|O-Phospho-L-tyrosine\|5,11-dimethyl-6H-pyrido[4,3- | |
| | b]carbazol-9-ol\|9-Methoxyellipticine\|9-Bromoellipticine\|4-(4-aminophenyl)-1H-indazol-3- | |
| | amine\|FIt-3 Inhibitor II\|6-(2,6-Dichlorophenyl)-2-{[3-(hydroxymethyl)phenyl]amino}-8- | |
| | methylpyrido[2,3-D]pyrimidin-7(8H)-one\|Pyrido(2,3-d)pyrimidin-7(8H)-one,6-(2,6- | |
| | dichlorophenyl)-2-((4-fluorophenyl)amino)-8-methyl-\|Floxuridine\|Motesanib\|2-((2- | |
| | Bromoethyl)(2,4-dinitro-6-((2-(phosphonooxy)ethyl)carbamoyl)phenyl)amino)ethyl | |
| | methanesulfonate\|Osi-930\|Melitracen\|1-Benzyl-3,5-dimethylpiperidin-4-one\|Amuvatinib\|5- | |
| | Methoxytryptoline\|Famitinib\|Ripretinib\|Pexidartinib\|Imatinib\|Semaxanib\|Sorafenib\|Regorafenib\| | |
| | Pazopanib hydrochloride\|Ponatinib\|H-DL-Tyr(2,6-diMe)-DL-Tic-DL-Lys(Cbz)-NHPh\|2-chloro-6- | |
| | methyl-N′-[4-methyl-3-[(4-pyridin-3-ylpyrimidin-2-yl)amino]benzoyl]benzohydrazide\|2,4″- | |
| | Dimethoxy-5,3″-dipropyl-biphenyl\|N-[2-(3,4-dihydro-2H-quinolin-1-yl)-2-oxoethyl]-2-[3-[(Z)- | |
| | hydroxyiminomethyl]phenyl]carbamoylamino]-N-quinolin-8-ylacetamide\|Tinidazole\|2-(3- | |
| | Chlorophenyl)histamine\|2-iodo-6-methyl-N′-[4-methyl-3-[(4-pyridin-3-ylpyrimidin-2- | |
| | yl)amino]benzoyl]benzohydrazide | |
| EPHA8 | 1H-Imidazo[2,1-f]purine-2,4(3H,8H)-dione,7-(5-hydroxy-2-methylphenyl)-8-(2-methoxyphenyl)- | Inhibitor |
| | 1-methyl-\|(2S)-2-[[(4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid | |
| CDK7 | N-[3-[5-chloro-4-(1H-indol-3-yl)pyrimidin-2-yl]amino]phenyl]-4-[(E)-4-(dimethylamino)but-2- | Inhibitor |
| | enoyl]amino]benzamide\|Cdk/Crk Inhibitor\|4-(6-Cyclohexylmethoxy-9H-purin-2-ylamino)-N,N- | |
| | diethylbenzamide\|Phosphonothreonine\|3,4-Dihydro-6-[4-[3- | |
| | (methylsulfonyl)phenyl]methyl]amino]-5-(trifluoromethyl)-2-pyrimidinyl]amino]-2(1H)- | |
| | quinolinone\|2,5-dichloro-N-p-tolylthiophene-3-sulfonamide\|(4-Amino-2-((1- | |
| | (methylsulfonyl)piperidin-4-yl)amino)pyrimidin-5-yl)(2,3-difluoro-6- | |
| | methoxyphenyl)methanone\|ZK-Cdk\|N-(5-(((5-(1,1-Dimethylethyl)-2-oxazolyl)methyl)thio)-2- | |
| | thiazolyl)-4-piperidinecarboxamide\|Mevociclib\|Olorofim\|Cdk7-IN-2\|N-(1-((2- | |
| | (Dimethylamino)ethyl)amino)-2-methyl-1-oxopropan-2-yl)-4-(4-(2-methyl-5-((2S,3R,4R,5S,6R)- | |
| | 3,4,5-trihydroxy-6-(methylthio)tetrahydro-2H-pyran-2-yl)benzyl)phenyl)-butanamide\|Seliciclib\|4- | |
| | [4-[2-[5-[(5-tert-butyl-1,3-oxazol-2-yl)methylsulfanyl]-1,3-thiazol-2-yl]amino]-2- | |
| | oxoethyl]phenoxy]-N-hydroxybutanamide\|N-[(1S)-1-phenylethyl]-2-piperidin-3-yloxy-8-propan-2- | |
| | ylpyrazolo[1,5-a][1,3,5]triazin-4-amine\|5,6,7-Tribromo-2-piperazin-1-yl-1,3- | |
| | diazatricyclo[6.3.1.04,12]dodeca-2,4,6,8(12)-tetraene\|2-N-(4-aminocyclohexyl)-9-cyclopentyl-6- | |
| | N-[6-(furan-2-yl)pyridin-3-yl]methyl]purine-2,6-diamine\|(R)-(-)-N-propyl-2-methoxy-11- | |
| | hydroxynoraporphine\|N-[(2-imidazol-1-ylphenyl)methyl]-2-(1-methylpiperidin-4-yl)oxy-8-propan- | |
| | 2-ylpyrazolo[1,5-a][1,3,5]triazin-4-amine | |
| FGFR2 | 1-(2-Chlorophenyl)-N-methyl-N-(1-methylpropyl)-3-isoquinolinecarboxamide\|5-Hydroxyindole-3- | Binder\|.\| |
| | acetic acid\|Actb-1003\|Platelet factor 4 (59-70)\|Unii-3mae0mxi6K\|7-(4-fluoroanilino)-1-[(1R,3R)-3- | Inhibitor\| |
| | hydroxycyclopentyl]-3-(4-methoxyphenyl)-4H-pyrimido[4,5-d]pyrimidin-2-one\|8- | Antagonist\| |
| | Bicyclo[2.2.1]hept-2-yl-2-{4-[4-(3-hydroxypropyl)piperidin-1-yl]phenylamino}-8H-pyrido[2,3- | Modulator\| |
| | d]pyrimidin-7-one\|Lofentanil\|Deuruxolitinib\|Alofanib\|MK-2461\|(R)-6-(4-((4-Ethylpiperazin-1- | Activator |
| | yl)methyl)phenyl)-N-(1-phenylethyl)-7H-pyrrolo[2,3-d]pyrimidin-4-amine\|Debio-1347\|1H- | |
| | Pyrazole-1-ethanol,4-((1E)-2-(5-((1R)-1-(3,5-dichloro-4-pyridinyl)ethoxy)-1H-indazol-3- | |
| | yl)ethenyl)-\|1-(6-Fluoroindol-1-yl)propan-2-amine\|Lucitanib\|Pemigatinib\|3′-Deoxy-3′-(1,2,3- | |
| | triazol-1-yl)thymidine\|3-tert-butyl-9H-carbazole | |
| SRPK2 | 3-(1H-indol-5-yl)-5-(4-((4-methylpiperazin-1-yl)methyl)phenyl)-1H-pyrrolo[2,3-b]pyridine | Inhibitor |
| MK01 | 2-(2-chloro-4-iodophenylamino)-N-cyclopropylmethoxy-3,4-difluorobenzamide\|(R)-3-(4- | Inhibitor\| |
| | isopropylphenyl)-7-methyl-N-(1-phenoxypropan-2-yl)pyrazolo[1,5-a]pyrimidine-6- | Activator\| |
| | carboxamide\|(R)-1-(2-oxo-2-(4-(4-(pyrimidin-2-yl)phenyl)piperazin-1-yl)ethyl)-N-(3-(pyridin-4-yl)- | Modulator |
| | 1H-indazol-5-yl)pyrrolidine-3-carboxamide\|ERK Inhibitor III\|Phosphonothreonine\|5-(2- | |
| | Phenylpyrazolo[1,5-A]pyridin-3-YL)-1H-pyrazolo[3,4-C]pyridazin-3-amine\|4-[[(2S)-2-(3- | |
| | Chlorophenyl)-2-hydroxyethyl]amino]-3-[7-methyl-5-(4-morpholinyl)-1H-benzimidazol-2-yl]-2(1H)- | |
| | pyridinone\|N-(4-fluorophenyl)-9-methylpurin-6-amine\|Bisindolylmaleimide | |
| | i\|Debromohymenialdisine\|Bisindolylmaleimide IV\|(S)-4-(2-(N-methylisoquinoline-5-sulfonamido)- | |
| | 3-oxo-3-(4-phenylpiperazin-1-yl)propyl)phenyl isoquinoline-5-sulfonate\|4,5,6,7- | |
| | Tetrabromobenzotriazole\|#N/A\|bisindolylmaleimide IX\|7-(4-fluoroanilino)-1-[(1R,3R)-3- | |
| | hydroxycyclopentyl]-3-(4-methoxyphenyl)-4H-pyrimido[4,5-d]pyrimidin-2-one\|KT5720\|4-(4- | |
| | Fluorophenyl)-1-(4-piperidinyl)-5-(2-amino-4-pyrimidinyl)-imidazole\|1-(((Z)-(1-(2-aminothiazol-4- | |
| | yl)-2-oxo-2-(((3S,4R)-2-oxo-4-((2-oxooxazolidin-3-yl)methyl)-1-sulfoazetidin-3- | |
| | yl)amino)ethylidene)amino)oxy)cyclopropanecarboxylic acid\|6-((2-((4-(2,4-Dichlorophenyl)-5-(4- | |
| | methyl-1H-imidazol-2-yl)pyrimidin-2-yl)amino)ethyl)amino)nicotinonitrile\|6-(3′,5′-Dimethylbenzyl)- | |
| | 1-ethoxymethyl-5-isopropyluracil\|Ravoxertinib\|Temuterkib\|Cilofexor\|Mitoquinone\|(3S)-3-[[(2S)-2- | |
| | [3-(methoxycarbonylamino)-2-oxopyridin-1-yl]butanoyl]amino]-4-oxo-5-(2,3,5,6- | |
| | tetrafluorophenoxy)pentanoic acid\|Ulixertinib | .\|Inhibitor\| |
| PGFRB | L-cis-diltiazem\|1-(4-((6,7-Dimethoxyquinolin-4-yl)oxy)-2-methoxyphenyl)-3-(1-(thiazol-2- | Modulator |
| | yl)ethyl)urea\|PDGF Receptor Tyrosine Kinase Inhibitor III\|PDGF RTK inhibitor\|FIt-3 Inhibitor II\|3- | |
| | Phenyl-1H-benzofuro[3,2-c]pyrazole\|1H-Imidazo(4,5-g)quinoxaline,1,2-dimethyl-6-(2-thienyl)-\|3- | |
| | [(4-chlorophenyl)-(1H-imidazol-2-yl)methylidene]-5-[(1-ethylpiperidin-4-yl)amino]-1H-indol-2- | |
| | one\|1-(2-(5-(2-Methoxyethoxy)benzimidazol-1-yl)quinolin-8-yl)piperidin-4-ylamine\|#N/A\|4-[(2S)- | |
| | 2-(3-Chlorophenyl)-2-hydroxyethyl]amino]-3-[7-methyl-5-(4-morpholinyl)-1H-benzimidazol-2-yl]- | |
| | 2(1H)-pyridinone\|(2,4-Dihydroindeno[1,2-c]pyrazol-3-yl)phenylamine\|N-(3-fluorophenyl)-6,7- | |
| | dimethoxy-1,4-dihydroindeno[1,2-c]pyrazol-3-amine\|5-[(Z)-(5-chloro-2-oxo-1,2-dihydro-3H-indol- | |
| | 3-ylidene)methyl]-N-[2-(diethylamino)ethyl]-2,4-dimethyl-1H-pyrrole-3-carboxamide\|4-(3-Bromo- | |
| | phenoxy)-6,7-dimethoxy-quinazoline\|4-(3-Bromo-phenoxy)-6,7-dimethoxy-quinoline\|6,7- | |
| | Dimethoxy-4-(3,4-dimethoxyphenoxy)quinoline\|6,7-dimethoxy-N-methyl-N-phenylquinazolin-4- | |
| | amine; hydrochloride\|6,7-Dimethoxy-3-pyridin-3-yl-quinoline\|4-(6,7-Dimethoxy-quinolin-3-yl)- | |
| | benzoic acid\|6,7-Dimethoxy-3-(4-methoxy-phenyl)-quinoline\|7-Fluoro-3-thiophen-3-yl- | |
| | quinoline\|6,7-Dimethoxy-3-thiophen-2-yl-quinoline\|7-Methoxy-3-thiophen-3-yl-quinoline\|3-(3,4- | |
| | Dimethoxy-phenyl)-6,7-dimethoxy-quinoline\|4-(6,7-Dimethoxyquinolin-3-yl)phenol\|7-Thiophen-3- | |
| | yl-[1,3]dioxolo[4,5-g]quinoline\|6,7-Difluoro-3-thiophen-3-yl-quinoline\|6,7-Dimethoxy-3-p-tolyl- | |
| | quinoline\|6,7-Dimethoxy-3-(3-methoxy-phenyl)-quinoline\|6,7-Dimethoxy-3-phenylethynyl- | |
| | quinoline\|3-Cyclopentyl-6,7-dimethoxy-quinoline\|6-Methoxy-3-thiophen-3-yl-quinoline\|3-(2- | |
| | Cyclohexyl-ethyl)-6,7-dimethoxy-quinoline\|7-Methoxy-3-pyridin-4-yl-quinoline\|6,7-Dimethoxy-3- | |
| | ((E)-styryl)-quinoline\|3-Pyridin-3-yl-quinoline-6,7-diol\|Benzyl-(6,7-dimethoxy-quinolin-3-yl)- | |
| | amine\|5,7-Dimethoxy-3-thiophen-3-yl-quinoline\|3-Cyclopent-1-enyl-6,7-dimethoxy-quinoline\|3-(4- | |
| | Pyridinyl)quinoline\|3-(3-Fluoro-phenyl)-6,7-dimethoxy-quinoline\|5,7-Dimethyl-3-thiophen-3-yl- | |
| | quinoline\|3-Benzyloxy-6,7-dimethoxy-quinoline\|6,7-Dimethoxy-3-phenyl-quinoline\|6,7- | |
| | Dimethoxy-3-thiophen-3-yl-quinoline\|5-(6,7-Dimethoxy-quinolin-3-yl)-1H-pyridin-2-one\|3-(3,4- | |
| | Difluoro-phenyl)-6,7-dimethoxy-quinoline\|3-(4-Fluoro-phenyl)-6,7-dimethoxy-quinoline\|3-(3,4- | |
| | Dichloro-phenyl)-6,7-dimethoxy-quinoline\|3-(3-Thienyl)quinoline\|3-((E)-Styryl)-quinoline\|6- | |
| | Methoxy-3-pyridin-4-yl-quinoline\|7-Chloro-3-pyridin-4-yl-quinoline\|5,7-Dimethoxy-3-(4- | |
| | pyridinyl)quinoline\|3-(1H-indol-3-yl)-6,7-dimethoxy-quinoline\|3-(1H-indol-3-yl)quinoline\|3- | |
| | Dimethoxy-3-(4-nitro-phenyl)-quinoline\|5,6,7-Trimethoxy-3-pyridin-4-yl-quinoline\|3-Pyridin-4-yl- | |
| | Cyclohexylethynyl-6,7-dimethoxy-quinoline\|6,7-Dichloro-3-thiophen-3-yl-quinoline\|6,7- | |
| | quinoline-5,7-diol\|6,7-Dimethoxy-3-pyridin-4-yl-quinoline\|3-Pyridin-4-yl-quinolin-7- | |
| | ol\|Bis(benzo[b]furan-2-yl)methanone\|(benzo[b]furan-2-yl)-(1H-indol-2-yl)methanone\|Bis(5- | |
| | aminobenzo[b]furan-2-yl)methanone\|Bis(5-acetoxybenzo[b]furan-2-yl)methanone\|Bis(6- | |
| | hydroxybenzo[b]furan-2-yl)methanone\|Bis(5-hydroxybenzo[b]furan-2-yl)methanone\|Unii- | |
| | 3mae0mxi6K\|6-(2,6-dichlorophenyl)-8-methyl-2-((3-(methylthio)phenyl)amino)pyrido[2,3- | |
| | d]pyrimidin-7(8H)-one\|(5-fluoro-1H-indol-2-yl)-(1H-indol-2-yl)methanone\|Bis(1H-2- | |
| | indolyl)methanone 12\|(1H-indol-2-yl)(5-phenoxy-1H-indol-2-yl)methanone\|Bis(1H-2- | |
| | indolyl)methanone 11\|(5-hydroxy-1H-indol-2-yl)-(1H-indol-2-yl)methanone\|di(1H-indol-2- | |
| | yl)methanone\|3-(1H-Benzimidazol-2-YL)-1H-indazole\|7-(4-fluoroanilino)-1-[(1R,3R)-3- | |
| | hydroxycyclopentyl]-3-(4-methoxyphenyl)-4H-pyrimido[4,5-d]pyrimidin-2-one\|6-(2,6- | |
| | Dichlorophenyl)-2-{[3-(hydroxymethyl)phenyl]amino}-8-methylpyrido[2,3-D]pyrimidin-7(8H)- | |
| | one\|6-(2,6-Dichlorophenyl)-8-methyl-2-(4-morpholin-4-ylanilino)pyrido[2,3-d]pyrimidin-7- | |
| | one\|Pyrido(2,3-d)pyrimidin-7(8H)-one,6-(2,6-dichlorophenyl)-2-((4-fluorophenyl)amino)-8- | |
| | methyl-\|2-[(4-Ethoxyphenyl)amino]-8-methyl-6-(2,6-dichlorophenyl)pyrido[2,3-d]pyrimidine- | |
| | 7(8H)-one\|2-[4-[2-[Ethyl(acetyl)amino]ethoxy]phenyl]amino]-8-methyl-6-(2,6- | |
| | dichlorophenyl)pyrido[2,3-d]pyrimidine-7(8H)-one\|6-(2,6-Dichlorophenyl)-2-[(4-Fluoro-3- | |
| | Methylphenyl)amino]-8-Methylpyrido[2,3-D]pyrimidin-7(8h)-One\|3-(4-Dimethylamino- | |
| | benzylidenyl)-2-indolinone\|4-(1H-benzimidazol-1-yl)aniline\|1H-Benzimidazole,5-methoxy-1- | |
| | phenyl-\|1-Phenyl-5-hydroxybenzimidazole\|5-Substituted 1-Phenylbenzimidazole 22\|6,7- | |
| | Dimethoxy-4-phenoxy-quinoline\|4-(3-Ethoxy-phenoxy)-6,7-dimethoxy-quinoline\|6,7-Dimethoxy- | |
| | 4-(2-methoxyphenoxy)quinoline\|6,7-Dimethoxy-4-(3-nitro-phenoxy)-quinoline\|6,7-Dimethoxy-4- | |
| | m-tolyloxy-quinoline\|3-(6,7-Dimethoxy-quinolin-4-yloxy)-phenylamine\|4-(3-Fluoro-phenoxy)-6,7- | |
| | dimethoxy-quinoline\|6,7-Dimethoxy-4-(4-methoxyphenoxy)quinoline\|4-(3-Ethyl-phenoxy)-6,7- | |
| | dimethoxy-quinoline\|4-(2,3-Dimethoxy-phenoxy)-6,7-dimethoxy-quinoline\|4-(3,5-Dimethoxy- | |
| | phenoxy)-6,7-dimethoxy-quinoline\|3-(6,7-Dimethoxy-quinolin-4-yloxy)-phenol\|6,7-Dimethoxy-4- | |
| | (3-methoxyphenoxy)quinoline\|Bis(5-methoxybenzo[b]furan-2-yl)methanone\|1-Phenyl-1H- | |
| | benzoimidazole\|3-benzyl-N-(2-morpholinoethyl)quinoxalin-2-amine\|[2- | |
| | (Octadecoxymethyl)oxolan-2-yl]methyl 2-(trimethylazaniumyl)ethyl phosphate\|1-[1-[4-(1- | |
| | acetylpiperidin-4-yl)oxy-2-methoxybenzoyl]piperidin-4-yl]-4H-3,1-benzoxazin-2-one\|Tak-593\|MK- | |
| | 2461\|Indiplon\|5-Methoxytryptoline\|Famitinib\|Lucitanib\|Semaxanib\|Rolipram\|Sorafenib\|(Z)-3-(3,4- | |
| | Dimethoxy-phenyl)-2-pyridin-3-yl-acrylonitrile\|6,7-Dimethyl-2-phenylquinoxaline\|Sunitinib\|H-DL- | |
| | Tyr(2,6-diMe)-DL-Tic-DL-Lys(Cbz)-NHPh\|2-chloro-6-methyl-N′-[4-methyl-3-[(4-pyridin-3- | |
| | ylpyrimidin-2-yl)amino]benzoyl]benzohydrazide\|2-iodo-6-methyl-N′-[4-methyl-3-[(4-pyridin-3- | |
| | ylpyrimidin-2-yl)amino]benzoyl]benzohydrazide | |
| KPCZ | Go-6976\|Bisindolylmaleimide i\|Bisindolylmaleimide IV\|(18S)-18-(hydroxymethyl)-17-oxa-4,14,21- | Inhibitor\|. |
| | triazahexacyclo[19.6.1.17,14.02,6.08,13.022,27]nonacosa-1(28),2(6),7(29),8,10,12,22,24,26- | |
| | nonaene-3,5-dione\|Phenol,4,4′-(3-methyl-1-butenylidene)bis-\|(E)-3-(4-Hydroxyphenyl)-2,3- | |
| | diphenylacrylonitrile\|4,4′-(Cycloheptylidenemethylene)diphenol\|2-(4-Hydroxyphenyl)-3,3- | |
| | diphenylacrylonitrile\|3,3-Bis(4-methoxyphenyl)-2-phenylpropenenitrile\|2,3,3- | |
| | Triphenylacrylonitrile\|3,3-Bis(4-hydroxyphenyl)-2-phenylprop-2-enenitrile\|Cyclofenil diphenol\|3- | |
| | (1-Methyl-1H-indol-3-yl)-4-[6,7,8,9-tetrahydro-12-methyl-7beta,8alpha- | |
| | (methaniminomethano)pyrido[1,2-a]indol]-10-yl]-1H-pyrrole-2,5-dione\|2,2′:5′,2″-Terthiophene-4- | |
| | carbaldehyde\|2,2′:5′,2″-Terthiophene-4,5″-dicarbaldehyde\|2,2′:5′,2″-Terthiophene-4- | |
| | methanol\|17-(Hydroxymethyl)-18-oxa-4,14,22- | |
| | triazahexacyclo[20.6.1.17,14.02,6.08,13.023,28]triaconta-1(29),2(6),7(30),8,10,12,23,25,27- | |
| | nonaene-3,5-dione\|(S)-3-(8-(Dimethylaminomethyl)-6,7,8,9-tetrahydropyrido(1,2-a)indol-10-yl)-4- | |
| | (1-methyl-3-indolyl)-1H-pyrrole-2,5-dione hydrochloride\|Methyl 2-amino-4-phenylthiophene-3- | |
| | carboxylate\|Benzyl 2-amino-4-(3,4-dimethoxyphenyl)thiophene-3-carboxylate\|Isopropyl 2-amino- | |
| | 4-(3,4-dimethoxyphenyl)thiophene-3-carboxylate\|6-(4-fluorophenyl) sulfanyl-2-(1H-indol-5- | |
| | ylamino)-8-methylpyrido[2,3-d]pyrimidin-7-one\|3-[1-[3-(Dimethylamino)propyl]-5-methoxy-1H- | |
| | indol-3-yl]-4-(1H-indol-3-yl)-1H-pyrrole-2,5-dione\|4,4′-(1E,1′E)-2,2′-(1-Phenyl-1H-pyrazole-3,5- | |
| | diyl)bis(ethene-2,1-diyl)bis(2-methoxyphenol)\|Methyl 4-(4- | |
| | hydroxybenzylideneamino)benzoate\|5-(4-Chlorophenyl)-1-(2,4-dichlorophenyl)-4-methylsulfinyl- | |
| | N-(piperidin-1-yl)-1H-pyrazole-3-carboxamide\|Lactitol\|5H-Cyclopenta(b)pyridine-6-carboxylic | |
| | acid,5-(1,3-benzodioxol-5-yl)-2-butyl-7-(2-((2S)-2-carboxypropyl)-4-methoxyphenyl)-6,7-dihydro- | |
| | ,(5S,6R,7R)-\|Balanol | |
| PLK2 | [4-((1Z)-2-(Acetylamino)-3-{[1-(1,1′-biphenyl-4-ylmethyl)-2-oxoazepan-3-YL]amino}-3-oxoprop-1- | Inhibitor |
| | enyl)-2-formylphenyl]acetic acid\|(7R)-8-cyclopentyl-7-ethyl-5-methyl-2-[5-(4-methylpiperazin-1- | |
| | yl)-2-(trifluoromethoxy)anilino]-7H-pteridin-6-one | |
| PKN1 | 1-(3-Hydroxybenzyl)-3-(4-(pyridin-4-yl)thiazol-2-yl)urea\|Spiro[piperidine-4,3′-[3H]pyrido[3,4- | Inhibitor |
| | blindole]-4′-carbonitrile,7′,8′-dichloro-1′,2′,4′,9′-tetrahydro-9′-methyl-1′-oxo- | |
| BMPR2 | Pergolide\|3-amino-5-[[(2S)-2-amino-3-[(2S)-1-[(2S)-1-[(2R,3R)-4-(3-carboxyanilino)-3-hydroxy- | Agonist\| |
| | 4-oxo-1-phenylsulfanylbutan-2-yl]amino]-4-methyl-1-oxopentan-2-yl]amino]-3-methyl-1- | Inhibitor |
| | oxobutan-2-yl]amino]-3-oxopropyl]carbamoyl]benzoic acid\|Nelipepimut-S\|1-[Bis(4- | |
| | chlorophenyl)methyl]-3-(4-chlorophenyl)-\|1-(4-(6-Methyl-5-(3,4,5-trimethoxyphenyl)pyridin-3- | |
| | yl)phenyl)piperazine\|5-[6-(4-Methoxyphenyl)pyrazolo[1,5-a]pyrimidin-3-yl]quinoline\|4-(4-(3-(1H- | |
| | Pyrazol-4-yl)pyrazolo[1,5-a]pyrimidin-6-yl)phenyl)morpholine\|4-[4-(3-Quinolin-4-ylpyrazolo[1,5- | |
| | a]pyrimidin-6-yl)phenyl]morpholine\|4-[6-[4-(4-Methylpiperazin-1-yl)phenyl]pyrazolo[1,5- | |
| | a]pyrimidin-3-yl]quinoline | |
| TRPM6 | 2-Aminoethyl diphenylborinate | Activator |
| MELK | Otssp 167 | Inhibitor |
| CHK1 | 2-(2-chloro-4-iodophenylamino)-N-cyclopropylmethoxy-3,4-difluorobenzamide\|1-(2-(3- | Inhibitor\| |
| | Aminopropoxy)phenyl)-3-(pyrazin-2-yl)urea\|CB-5083\|4-chloro-3-[(3-chloropyridin-2-yl)-(2- | Modulator |
| | hydroxyethyl)amino]-N-[4-(2-methylpropoxy)phenyl]benzamide\|8-(2-Ethyl-1-methyl-butoxy)- | |
| | quinolin-2-ylamine\|Gatifloxacin\|9,10,11,12-Tetrahydro-9,12-epoxy-1H-diindolo[1,2,3-fg:3′,2′,1′- | |
| | kl]pyrrolo[3,4-i][1,6]benzodiazocine-1,3(2H)-dione\|7-Chloro-3-oxo-8-(1,3-thiazol-5- | |
| | ylmethylamino)-11,16-dioxa-2,4,19,21-tetrazatricyclo[15.3.1.05,10]henicosa- | |
| | 1(20),5,7,9,17(21),18-hexaene-18-carbonitrile\|3-((R)-1-((2S,6R)-2,6-dimethylmorpholino)ethyl)- | |
| | N-(6-methyl-3-(1H-pyrazol-4-yl)imidazo[1,2-a]pyrazin-8-yl)isothiazol-5-amine\|N-[3-[5-Iodo-4-[[3- | |
| | [(2-thienylcarbonyl)amino]propyl]amino]-2-pyrimidinyl]amino]phenyl]-1- | |
| | pyrrolidinecarboxamide\|N-[3-[5-Bromo-4-[2-(1H-imidazol-5-YL)ethyl]amino]-2- | |
| | pyrimidinyl]amino]phenyl]-1-pyrrolidinecarboxamide\|2-[3,4-Dioxo-2-(pyridin-4- | |
| | ylamino)cyclobuten-1-yl]amino]-2-(3-hydroxyphenyl)acetamide\|3-[(2R)-1- | |
| | (dimethylamino)propan-2-yl]oxy-5-[4-methoxy-5-(1-methylpyrazol-4-yl)pyridin-2- | |
| | yl]amino]pyrazine-2-carbonitrile\|2-Quinolinone deriv. 3\|2-(Cyclohexylamino)benzoic acid\|(5,6- | |
| | Diphenyl-furo[2,3-d]pyrimidin-4-ylamino)-acetic acid\|3-(1H-Benzimidazol-2-YL)-1H-indazole\|6- | |
| | Morpholin-4-Yl-9h-Purine\|Bisindolylmaleimide i\|Debromohymenialdisine\|Bisindolylmaleimide | |
| | IV\|6-(3-aminopropyl)benzo[h]isoquinolin-1(2H)-one\|9-chlorobenzo[h]isoquinolin-1(2H)-one\|(S)-4- | |
| | (2-(N-methylisoquinoline-5-sulfonamido)-3-oxo-3-(4-phenylpiperazin-1-yl)propyl)phenyl | |
| | isoquinoline-5-sulfonate\|4,5,6,7-Tetrabromobenzotriazole\|sogranulatimide\|Bis-imide | |
| | A\|Granulatimide\|9-hydroxypyrrolo[3,4-c]carbazole-1,3(2H,6H)-dione\|(3Z)-6-(4-Hydroxy-3- | |
| | methoxyphenyl)-3-(1H-pyrrol-2-ylmethylene)-1,3-dihydro-2H-indol-2-one\|06- | |
| | Cyclohexylmethoxy-2-(4′-sulphamoylanilino)purine\|#N/A\|Rel-(9R,12S)-9,10,11,12-tetrahydro- | |
| | 9,12-epoxy-1H-diindolo[1,2,3-FG:3′,2′,1′-KL]pyrrolo[3,4-I][1,6]benzodiazocine-1,3(2H)-dione\|1- | |
| | Benzyl-N-(5-{5-[3-(Dimethylamino)-2,2-Dimethylpropoxy]-1h-Indol-2-Yl}-6-Oxo-1,6- | |
| | Dihydropyridin-3-Yl)-1h-Pyrazole-4-Carboxamide\|PF-477736\|N-Feruloyl dopamine,trans-\|(S)-5- | |
| | (3-Fluorophenyl)-N-(piperidin-3-yl)-3-ureidothiophene-2-carboxamide\|5-((1-Ethyl-4- | |
| | piperidinyl)oxy)-9H-pyrrolo(2,3-b:5,4-c′)dipyridine-6-carbonitrile\|(E)-3-(2-chloro-4-((3-(2,6- | |
| | dichlorophenyl)-5-isopropylisoxazol-4-yl)methoxy)styryl)benzoic acid\|6-bromo-3-(1-methyl-1H- | |
| | pyrazol-4-yl)-5-(piperidin-3-yl)pyrazolo[1,5-a]pyrimidin-7-amine\|LY 2880070[Who-DD]\|7- | |
| | Hydroxystaurosporine\|Rabusertib\|(R)-6-bromo-3-(1-methyl-1H-pyrazol-4-yl)-5-(piperidin-3- | |
| | yl)pyrazolo[1,5-a]pyrimidin-7-amine\|Prexasertib\|2-N-(4-piperazin-1-ylpyridin-3-yl)-4-N-pyridin-3- | |
| | yl-1,3-thiazole-2,4-dicarboxamide | |
| JAK1 | Ethyl 6-bromo-4-hydroxyquinoline-3-carboxylate\|2-Bromo-4-[(6,7-dimethoxyquinazolin-4- | Inhibitor\| |
| | yl)amino]phenol\|3-[Benzyl(propan-2-yl)amino]-1-(naphthalen-2-yl)propan-1-one\|2-N-((1S)-1-(5- | Modulator |
| | Fluoropyrimidin-2-yl)ethyl)-4-N-(1-methylimidazol-4-yl)-7H-pyrrolo(2,3-d)pyrimidine-2,4- | |
| | diamine\|CF53 free base\|Itacitinib\|Solcitinib\|Filgotinib\|Cerdulatinib\|(5Z)-5-[4-[(1-pyridin-2- | |
| | ylpyrrolidin-2-yl)methoxy]phenyl]methylidene]-1,3-thiazolidine-2,4- | |
| | dione\|Brepocitinib\|Deuruxolitinib\|Ifidancitinib\|Simufilam\|Peficitinib\|Upadacitinib\|Ruxolitinib\| | |
| | Baricitinib\|N-(6-phenyl-1H-indazol-3-yl)butyramide\|Cdk7-IN-2\|3-(2-(Pyridin-4-yl)ethyl)-1H- | |
| | indole\|Balapiravir\|H-Gly-Gly-Thr-Thr-Gly-Gly-Thr-Gly-Thr-Gly-Gly-Thr-Thr-Gly-Gly- | |
| | OH\|13,16,19,22-Tetraoxa-3,6-diazatricyclo[21.3.1.18,12]octacosa-1(27),2,6,8(28),9,11,23,25- | |
| | octaene-27,28-diol\|Acetic acid 2-heptylselanyl-phenyl ester\|Nicotinamide riboside\|(5-(3- | |
| | methoxyphenyl)-1-pentyl-1H-pyrrol-3-yl)(naphthalen-1-yl)methanone\|[(4R,4aR,7S,7aR,12bS)-9- | |
| | acetyloxy-3-methyl-1,2,3,4,4a,7,7a,13-octahydro-4,12-methanobenzofuro[3,2-e]isoquinolin-3- | |
| | ium-7-yl]sulfate\|Bifeprunox\|(3-(4-(6-Oxa-2-azaspiro[3.4]octan-2-ylmethyl)-3- | |
| | methylphenoxy)azetidin-1-yl)(5-phenyl-1,3,4-oxadiazol-2-yl)methanone\|N,N′-Bis(3- | |
| | (ethylamino)propyl)-1,7-heptanediamine\|Enadoline\|4-(tert-Butyl)-N-(2-methyl-3-(4-methyl-6-((4- | |
| | (morpholine-4-carbonyl)phenyl)amino)-5-oxo-4,5-dihydropyrazin-2-yl)phenyl)benzamide\|4-[3-(2- | |
| | Fluorophenoxy)phenyl]-1-phosphonatobutane-1-sulfonate\|Etiprednol dicloacetate\|(S)-5-(3- | |
| | Fluorophenyl)-N-(piperidin-3-yl)-3-ureidothiophene-2-carboxamide\|Crenigacestat\|Fluspirilene\|4- | |
| | [(Z)-1-(1-Naphthyl)-1-propenyl]-1H-imidazole\|Moxidectin\|1-Cyclohexyl-4-[3-(5-methoxy-1,2,3,4- | |
| | tetrahydronaphthalen-1-yl)propyl]piperazine\|N~4~-(Acridin-9-yl)-N~1~,N~1~-diethylbutane-1,4- | |
| | diamine\|1-[5-Ethyl-2-hydroxy-4-[6-methyl-6-(1H-tetrazol-5-YL)heptyl]oxy]phenyl]ethanone\|1- | |
| | ((3,5-Dibromophenyl)methyl)-1H-imidazole\|2-[4-(3-Methyl-2-thienyl)phenyl]propionic acid\|N-(5- | |
| | (3,5-difluorophenylthio)-1-H-pyrazolo[3,4-b]pyridine-3-yl)-4-(4-methylpiperazine-1-yl)-2- | |
| | (tetrahydro-2H-pyran-4-ylamino)benzamide\|Loviride\|7,3′,4′-Trihydroxyflavone\|5-Butyl-8-phenyl- | |
| | 3H-[1,2,4]triazolo[5,1-i]purine\|7 alpha,27-Dihydroxycholesterol\|6H-(1,2,4) Triazolo(4,3- | |
| | a)(1,5)benzodiazepine-6-acetamide,4,5-dihydro-4-(1H-indol-3-ylmethyl)-N-(1-methylethyl)-5- | |
| | oxo-1-phenyl-N-(phenylmethyl)-,(S)-\|8-Bromo-3-methyl-5-phenyl-2,3,4,5-tetrahydro-1H- | |
| | benzo[d]azepin-7-ol\|6-(4-Deoxy-4-((2E,4E)-tetradecadienoylglycyl)amino-L-glycero-b-L- | |
| | mannoheptopyranosyl)amino-9H-purine\|Indiplon\|Cyclofenil\|1-(3-carbamimidoylphenyl)-3-methyl- | |
| | N-[4-(2-sulfamoylphenyl)phenyl]-1H-pyrazole-5-carboxamide\|deamino-Cys(1)-nTyr-D-xille-D- | |
| | Gln-D-Asn-D-Cys(1)-D-Pro-Lys(Unk)-Unk\|N-(7-chloro-1-oxo-1,2-dihydroisoquinolin-6-yl)-2- | |
| | (cyclopropylamino)-2-phenylacetamide\|2H-Benzofuro(3,2-g)-1-benzopyran-2-one,4-methyl- | |
| | \|(2S)-N-dodecyl-1-[(2S)-pyrrolidine-2-carbonyl]pyrrolidine-2-carboxamide\|5,8-dichloro-7-[1-(2- | |
| | hydroxyacetyl)piperidin-4-yl]-methoxymethyl]-2-[(4-methoxy-6-methyl-2-oxo-1H-pyridin-3- | |
| | yl)methyl]-3,4-dihydroisoquinolin-1-one\|Urea,N-cyclopropyl-N′-(3-((1,2-dihydro-2-oxo-6- | |
| | quinolinyl)oxy)propyl)-N-((1R,2R)-2-hydroxycyclohexyl)-\|1-(2-Methoxyphenyl)-4- | |
| | propylpiperazine\|Thiorphan\|(R)-N-[(1S,2R)-2-Hydroxyindan-1-yl]-3-[(2S,3S)-3-(4- | |
| | methoxyphenoxyacetyl)amino-2-hydroxy-4-phenylbutanoyl]-5,5-dimethyl-1,3-thiazolidine-4- | |
| | carboxamide\|Tubercidin\|Rhein\|Dasantafil\|1-(4-Bromooxazol-2-yl)-7-phenylheptan-1-one\|2-[4-[2- | |
| | (diethylamino)ethoxy]phenyl]-N-[(2,4-difluorophenyl)methyl]-5-fluoropyrimidin-4-amine\|3-(1- | |
| | adamantyl)-6,7,8,9-tetrahydro-5H-[1,2,4]triazolo[4,3-a]azepine\|H-D-Cys(Bzl)-OH\|1-(3-Acetyloxy- | |
| | 17-(1-oxobutoxy)-2-(1-piperidinyl)-androstan-16-yl)-1-methylpiperidinium\|8-(1,3-oxazol-5-yl)-N- | |
| | phenyl-6-(trifluoromethyl)imidazo[1,2-a]pyridine-3-carboxamide\|WtInsagyllgphavgnhpsfsdknglts- | |
| | conh2\|2-Hydrazinylbenzenesulfonamide | |
| ARBK1 | 3-({[4-Methyl-5-(Pyridin-4-Yl)-4h-1,2,4-Triazol-3-Yl]methyl}amino)-N-[2- | Inhibitor |
| | (Trifluoromethyl)benzyl]benzamide\|5-(1-Piperidin-4-ylpyrazol-4-yl)-3-(6-pyrrolidin-1-yl-1,3- | |
| | benzoxazol-2-yl)pyridin-2-amine\|(4S)-5-(4-ethoxycarbonylpiperazin-1-yl)-4-[4-(1-ethoxy-2- | |
| | methyl-1-oxopropan-2-yl)oxy-7-methylquinoline-2-carbonyl]amino]-5-oxopentanoic | |
| | acid\|U.S. Pat No. 9,493,490, Example 3\|U.S. Pat No. 9,493,490, Example 4\|U.S. Pat No. 9,493,490, | |
| | Example 5\|U.S. Pat No. 9,493,490, Example 11\|4-[4-fluoro-3-(2-pyridin-4-ylethylcarbamoyl)phenyl]- | |
| | N-(1H-indazol-5-yl)-6-methyl-2-oxo-3,4-dihydro-1H-pyrimidine-5-carboxamide | |
| TBK1 | N-[3-[5-cyclopropyl-2-[3-(morpholin-4-ylmethyl)anilino]pyrimidin-4- | Inhibitor |
| | yl]amino]propyl]cyclobutanecarboxamide\|N-{3-[(5-cyclopropyl-2-{[4-(morpholin-4- | |
| | yl)phenyl]amino}pyrimidin-4-yl)amino]propyl}cyclobutanecarboxamide\|U.S. Pat No. 8,962,609, Dmx- | |
| | 16\|U.S. Pat No. 8,962,609, Dmx-14\|4-[[4-(3-cyano-4-pyrrolidin-1-ylphenyl)pyrimidin-2-yl]amino]-N-propan-2- | |
| | ylbenzamide\|21-Deoxycortisone\|4-[4-[3-cyano-4-(oxan-4-yloxy)phenyl]pyrimidin-2-yl]amino]-N- | |
| | [2-(dimethylamino)ethyl]-2-methoxy-N-methylbenzamide\|5-[2-[6-[2- | |
| | (Dimethylamino)ethylamino]pyridin-3-yl]amino]pyrimidin-4-yl]-2-(3-fluoropyrrolidin-1- | |
| | yl)benzonitrile\|3-[4-(3-Cyano-4-pyrrolidin-1-yl-phenyl)-pyrimidin-2-ylamino]-benzamide\|IKK | |
| | epsilon-IN-1\|2-(4-Butoxy-phenyl)-N-hydroxy-N-methyl-acetamide\|N-[4-[4-[3-cyano-4-(oxan-4- | |
| | yloxy)phenyl]pyrimidin-2-yl]amino]phenyl]methyl]acetamide\|Bardoxolone methyl\|5-[2-[4-[4-(2- | |
| | Hydroxyethyl)piperazin-1-yl]anilino]pyrimidin-4-yl]-3-methoxy-2-(oxan-4- | |
| | yloxy)benzonitrile\|U.S. Pat No. 8,962,609, Dmx-66\|N-[2-Cyano-4-(2-{[4-(morpholin-4- | |
| | yl)phenyl]amino}pyrimidin-4-yl)phenyl]-3-methylbutanamide | |
| ACK1 | N-phenethyl-5-phenyl-6-(4-(2-(pyrrolidin-1-yl)ethoxy)phenyl)furo[2,3-d]pyrimidin-4-amine\|4-(4- | Inhibitor |
| | Fluorophenyl)-3-(pyridin-4-yl)-1-(2,4,6-trichlorophenyl)-1H-pyrazol-5-amine\|3-[8-Amino-1-(4- | |
| | benzylphenyl)imidazo[1,5-a]pyrazin-3-yl]-1-methylcyclobutan-1-ol\|3-[8-Amino-1-[4- | |
| | [difluoro(phenyl)methyl]phenyl]imidazo[1,5-a]pyrazin-3-yl]-1-methylcyclobutan-1-ol\|3-[8-Amino-1- | |
| | [4-(1-hydroxy-1-phenylethyl)phenyl]imidazo[1,5-a]pyrazin-3-yl]-1-methylcyclobutan-1-ol | |
| DCLK2 | 2-{[2-ethoxy-4-(4-hydroxypiperidin-1-yl)phenyl]amino}-5,11-dimethyl-5,11-dihydro-6H- | Inhibitor |
| | pyrimido[4,5-b][1,4]benzodiazepin-6-one | |
| TAOK3 | (7S)-2-(2-aminopyrimidin-4-yl)-7-(2-fluoroethyl)-1,5,6,7-tetrahydro-4H-pyrrolo[3,2-c]pyridin-4-one | Inhibitor |
| IRAK1 | IRAK-1/4 Inhibitor\|2-[(2-aminoethyl)(methyl)amino]-N-(7-methoxyquinolin-6-yl)thieno[3,2- | Inhibitor |
| | d]pyrimidine-7-carboxamide\|N-{2-[4-(aminomethyl)piperidin-1-yl]-5-chlorophenyl}-6- | |
| | hydroxypyrazolo[1,5-a]pyrimidine-3-carboxamide\|[[(2R,3S,4R,5R)-5-(6-amino-2- | |
| | propylsulfanylpurin-9-yl)-3,4-dihydroxyoxolan-2-yl]methoxy-hydroxyphosphoryl]oxy- | |
| | hydroxyphosphoryl]-difluoromethyl]phosphonic acid\|4-[4-(3-Quinolin-4-ylpyrazolo[1,5- | |
| | a]pyrimidin-6-yl)phenyl]morpholine\|3-[4-[5-(4-Fluorophenyl)-2-pyrimidin-2-ylpyrazol-3- | |
| | yl]piperidin-1-yl]-2-phenylpropanoic acid\|Unk-D-Val-D-Pro-D-Val-CF2CF3 | |
| CDKL1 | 2-fluoro-4-(4-(1-isopropyl-2-methyl-1H-imidazol-5-yl)pyrimidin-2-ylamino)-N-methylbenzamide | Inhibitor |
| VGFR1 | 5-[(Z)-(5-chloro-2-oxo-1,2-dihydro-3H-indol-3-ylidene)methyl]-N-[2-(diethylamino)ethyl]-2,4- | Inhibitor\| |
| | dimethyl-1H-pyrrole-3-carboxamide\|2-(Pyridin-4-Ylmethylamino)-~{n}-[3- | Activator\| |
| | (Trifluoromethyl)phenyl]benzamide\|Piribedil\|N-[4-[5-(4-bromophenyl)-1-(4- | Modulator\| |
| | sulfamoylphenyl)pyrazol-3-yl]methyl]phenyl]benzamide\|1-[4-Amino-2,6-di(propan-2-yl)phenyl]-3- | |
| | [1-butyl-4-[3-(4-methylpentoxy)phenyl]-2-oxo-1,8-naphthyridin-3-yl]urea\|Cep- | |
| | 11981\|Zanoterone\|Foliglurax\|CID | |
| | 6509858\|Fascaplysin\|Famitinib\|Sulfatinib\|Lucitanib\|Fruquintinib\|Tivozanib\|3-[(4-chlorophenyl)- | |
| | (1H-imidazol-2-yl)methylidene]-5-[(1-ethylpiperidin-4-yl)amino]-1H-indol-2-one\|7-Chloro-3-oxo-8- | |
| | (1,3-thiazol-5-ylmethylamino)-11,16-dioxa-2,4,19,21-tetrazatricyclo[15.3.1.05,10]henicosa- | |
| | 1(20),5,7,9,17(21),18-hexaene-18-carbonitrile\|Cdk1/2 Inhibitor III\|Pyrazolo[1,5-a]pyrimidine | |
| | 1a\|N-[2,4-dichlorobenzoyl]phenylsulfonamide\|4-Chloro-N-(2- | |
| | chlorobenzoyl)benzenesulfonamide\|3-chloro-N-(4-chlorophenyl)sulfonylbenzamide\|4-Chloro-N- | |
| | [(4-chlorophenyl)sulfonyl]benzamide\|N-(4-chlorobenzenesulfonyl)-2-methylbenzamide\|N,5- | |
| | diphenyl-1,3-oxazol-2-amine\|2-[(5-Phenyl-1,3-oxazol-2-yl)amino]benzonitrile\|N-(2- | |
| | methoxyphenyl)-5-phenyl-1,3-oxazol-2-amine\|3-[(5-Phenyl-1,3-oxazol-2-yl)amino]benzonitrile\|3- | |
| | (5-Phenyl-oxazol-2-ylamino)phenylmethanol\|N-(3-methylphenyl)-5-phenyl-1,3-oxazol-2-amine\|3- | |
| | bromo-N-(4-chlorophenyl)sulfonylbenzamide\|N-(4-chlorophenyl)sulfonyl-4-nitrobenzamide\|4-[(5- | |
| | Phenyl-1,3-oxazol-2-yl)amino]benzenesulfonamide\|Lycogarubin B\|N-(4-chloro-2-fluorophenyl)- | |
| | 6,7-dimethoxyquinazolin-4-amine\|4-(4-(4-Tert-butylphenylamino)phthalazin-1- | |
| | yl)benzamide\|c[homoPhe-Hca-Glu-Gly-Leu-Glu-Glu]-NH2\|N3-GHQMFYYPra-NH2\|(4- | |
| | Chlorophenyl)-(4-isoquinolin-5-yl-phthalazin-1-yl)amine\|4-(isoquinolin-5-yl)-N-m-tolylphthalazin- | |
| | 1-amine\|4-(isoquinolin-5-yl)-N-o-tolylphthalazin-1-amine\|3-(1H-Benzimidazol-2-YL)-1H- | |
| | indazole\|N(1)Tyr-Tyr-Asp-Glu-Lys-Leu-Glu-Glu(1)-NH2\|c[YYAEGLEE]-NH2\|c[YYDEGLEE]- | |
| | NH2\|MK-2461\|2-Pyridin-2-yl-6H-pyrazolo[1,5-c]quinazolin-5-one\|Vatalanib\|Semaxanib | |
| ZAP70 | Syk Inhibitor II\|7-butyl-6-(4-methoxyphenyl)-5H-pyrrolo[2,3-b]pyrazine\|8-(4-morpholin-4- | Inhibitor |
| | ylanilino)-6-phenyl-2H-2,7-naphthyridin-1-one\|6-(3-aminopropyl)-8-(4-morpholin-4-ylanilino)-2H- | |
| | 2,7-naphthyridin-1-one\|6-(2-chlorophenyl)-8-(4-morpholin-4-ylanilino)-2H-2,7-naphthyridin-1-one | |
| SGK1 | 2-(2-chloro-4-iodophenylamino)-N-cyclopropylmethoxy-3,4-difluorobenzamide\|2-cyclopentyl-4- | Inhibitor |
| | (5-phenyl-1H-pyrrolo[2,3-b]pyridin-3-yl)benzoic acid\|Bisindolylmaleimide i\|Bisindolylmaleimide | |
| | IV\|(S)-4-(2-(N-methylisoquinoline-5-sulfonamido)-3-oxo-3-(4-phenylpiperazin-1-yl)propyl)phenyl | |
| | isoquinoline-5-sulfonate\|4,5,6,7-Tetrabromobenzotriazole\|N3-(4-fluorophenyl)-1h-pyrazolo[3,4- | |
| | d]pyrimidine-3,4-diamine | |
| MK14 | 2-(2-chloro-4-iodophenylamino)-N-cyclopropylmethoxy-3,4-difluorobenzamide\|1-(7-(4- | Inhibitor\| |
| | Fluorophenyl)-8-(pyridin-4-yl)-3,4-dihydropyrazolo[5,1-c][1,2,4]triazin-2(1H)-yl)-2-phenylethane- | Modulator |
| | 1,2-dione sulfate\|Ozagrel\|Tiazofurin adenine dinucleotide\|CID 195821\|2-(4-Benzylpiperazin-1- | |
| | yl)-1,3-benzothiazole\|4-butyl-N-(7-hydroxynaphthalen-1-yl)benzamide\|N-cyclopropyl-3-fluoro-4- | |
| | methyl-5-[3-(1-methylcyclopropyl)-[1,2,4]triazolo[4,3-a]pyridin-7-yl]benzamide\|3-(2-(Pyridin-4- | |
| | yl)ethyl)-1H-indole\|2-Amino-3-benzyloxypyridine\|2-Chlorophenol\|Triazolopyridine deriv. 25\|4- | |
| | Phenoxy-N-(pyridin-2-ylmethyl)benzamide\|3-(1-Naphthylmethoxy)pyridin-2-amine\|Octyl beta-D- | |
| | glucopyranoside\|4-(4-Fluorophenyl)-2-(4-hydroxyphenyl)-5-(4-pyridyl)-1H-imidazole\|N-(3- | |
| | (trifluoromethyl)benzyl)-4-phenoxybenzamide\|N-(3-(trifluoromethoxy)benzyl)-4- | |
| | phenoxybenzamide\|N-(4-(trifluoromethyl)benzyl)-4-phenoxybenzamide\|N-(4-methyl-benzyl)-4- | |
| | phenoxy-benzamide\|3-(dimethylamino)-N-(3-(4-hydroxybenzamido)-4- | |
| | methylphenyl)benzamide\|4-(2-Ethyl-4-m-tolyl-thiazol-5-yl)-pyridine\|N-(3-chlorophenyl)-6,7- | |
| | dimethoxyquinazolin-4-amine\|N-{3-[4-(3-Azido-phenyl)-5-pyridin-4-yl-imidazol-1-yl]-propyl}-4- | |
| | iodo-benzamide\|N,3-diphenyl-1H-indazol-6-amine\|Bisindolylmaleimide IV\|Purine,6-(phenylthio)- | |
| | \|6-(4-fluorophenyl)sulfanyl-7H-purine\|4-(Phenylsulfanyl)-7H-pyrrolo[2,3-d]pyrimidine\|Purine,6- | |
| | (benzylthio)-\|Bisindolylmaleimide i\|9-Benzyl-6-phenylsulfanylpurine\|9-Benzyl-6-(4- | |
| | fluorophenyl)sulfanylpurine\|9-[(4-Fluorophenyl)methyl]-6-phenylsulfanylpurine\|2-(4- | |
| | Fluorophenyl)-4-methoxy-3-(pyridin-4-YL)-1H-pyrrolo[2,3-B]pyridin-6-amine\|2-(Methylthio)-4-(4- | |
| | fluorophenyl)-5-(4-pyridinyl)-1H-imidazole\|4-[5-(4-Fluorophenyl)-2-(4-methanesulfinyl- | |
| | benzylsulfanyl)-3H-imidazol-4-yl]pyridine\|p38 MAP Kinase Inhibitor III\|(S)-4-(2-(N- | |
| | methylisoquinoline-5-sulfonamido)-3-oxo-3-(4-phenylpiperazin-1-yl)propyl)phenyl isoquinoline-5- | |
| | sulfonate\|Benzonitrile,3-(((5-(6-methyl-2-pyridinyl)-4-(6-quinoxalinyl)-1H-imidazol-2- | |
| | yl)methyl)amino)-\|4,5,6,7-Tetrabromobenzotriazole\|6-o-Tolylquinazolin-2-amine\|4-(4- | |
| | Fluorophenyl)-1-cycloropropylmethyl-5-(4-pyridyl)-imidazole\|(5-Amino-1-phenyl-1H-pyrazol-4- | |
| | YL)(phenyl)methanone\|[5-Amino-1-(4-fluorophenyl)-1H-pyrazol-4-YL](3-{[(2R)-2,3- | |
| | dihydroxypropyl]oxy}phenyl)methanone\|2-chloro-5-(2-phenyl-5-(pyridin-4-yl)-1H-imidazol-4- | |
| | yl)phenol\|#N/A\|4-(4-(3-(pyridin-2-yl)-1H-pyrazol-4-yl)pyridin-2-yl)-N-(tetrahydro-2H-pyran-4- | |
| | yl)benzamide\|bisindolylmaleimide IX\|6-(2,6-Dichlorophenyl)-2-{[3-(hydroxymethyl)phenyl]amino}- | |
| | 8-methylpyrido[2,3-D]pyrimidin-7(8H)-one\|Pyrido(2,3-d)pyrimidin-7(8H)-one,6-(2,6- | |
| | dichlorophenyl)-2-((4-fluorophenyl)amino)-8-methyl-\|4-(Fluorophenyl)-1-cyclopropylmethyl-5-(2- | |
| | amino-4-pyrimidinyl)imidazole\|3,4-Dihydro-2(1H)-quinolinone\|Benzamide,3-((4-(6-methyl-2- | |
| | pyridinyl)-5-(6-quinoxalinyl)-1H-imidazol-2-yl)methyl)-\|N-(4-fluorobenzyl)-N-(pyridin-4-yl)-2- | |
| | naphthamide\|Indiplon\|KT5720\|Talmapimod\|4-[3-(4-Fluorophenyl)-1h-Pyrazol-4-Yl]pyridine\|4-(4- | |
| | Fluorophenyl)-1-(4-piperidinyl)-5-(2-amino-4-pyrimidinyl)-imidazole\|6-(2,4-Difluorophenoxy)-8- | |
| | methyl-2-(tetrahydro-2h-pyran-4-ylamino)pyrido[2,3-d]pyrimidin-7(8h)-one\|4-(5-(4-Fluorophenyl)- | |
| | 3-(4-piperidyl)imidazol-4-yl)pyridine\|4-(4-(4-fluorophenyl)-1-(piperidin-4-yl)-1H-imidazol-5-yl)-2- | |
| | methoxypyrimidine\|Pamapimod\|Dilmapimod\|Neflamapimod\|VX-702\|Losmapimod\|4-(4- | |
| | Fluorophenyl)-2-(4-methylsulfinylphenyl)-5-(4-pyridyl)-1H-imidazole\|Metamizole\|4-(2-(3-tert- | |
| | Butylphenylamino)ethyl)-1H-imidazole\|[4-[8-tert-butyl-6-(4-fluorophenyl)imidazo[1,2-b]pyridazine- | |
| | 2-carbonyl]-3,3-dimethylpiperazin-1-yl]-(1H-pyrazol-5-yl)methanone\|ethyl (7S)-11-oxo-14-(2- | |
| | trimethylsilylethynyl)-2,4,10-triazatetracyclo[10.4.0.02,6.07,10]hexadeca-1(16),3,5,12,14- | |
| | pentaene-5-carboxylate\|Femoxetine\|3-(Benzo[d]oxazol-2-yl)phenyl propylcarbamate | |
| IKKA | S-(4-Cyanobenzyl)isothiourea hydrobromide\|Carafate\|Oxatomide\|5-Bromo-6-methoxy-beta- | Inhibitor\|. |
| | carboline\|N-(4-amino-5-cyano-6-phenylpyridin-2-yl)acetamide | |
| KPYM | N-(5-chloro-6-methyl-1,3-benzothiazol-2-yl)-2-phenylacetamide\|(4R,7S,10R,13S,16R)-7-(4- | Activator\| |
| | aminobutyl)-N-[(2S,3R)-1-amino-3-hydroxy-1-oxobutan-2-yl]-16-[(2R)-2-amino-3- | Modulator |
| | phenylpropanoyl]amino]-13-[(4-hydroxyphenyl)methyl]-10-(1H-indol-3-ylmethyl)-6,9,12,15- | |
| | tetraoxo-1,2-dithia-5,8,11,14-tetrazacycloheptadecane-4-carboxamide | |
| E2AK4 | 2-{[2-(4-{5-[(1-ethyl-1H-pyrazol-3-yl)amino]-1H-[1,2,3]triazolo[4,5-d]pyrimidin-1-yl}phenyl)propan- | Inhibitor |
| | 2-yl]oxy}ethan-1-ol | |
| AKT1 | 2-(2-chloro-4-iodophenylamino)-N-cyclopropylmethoxy-3,4-difluorobenzamide\|4-Amino-5,6,7,8- | Inhibitor\|.\| |
| | tetrahydro-5-oxo-8-(beta-D-ribofuranosyl)pyrido[2,3-d]pyrimidine-6-carboxamide\|4′-[1-(1H- | Modulator |
| | Imidazole-1-yl)propyl]biphenyl-3,4-diol[3H]Snap-7941\|2-Phenylamino-4-Methyl-5-Acetyl | |
| | Thiazole\|(2s)-1-{[5-(3-Methyl-1h-Indazol-5-Yl)pyridin-3-Yl]oxy}-3-Phenylpropan-2-Amine\|(2s)-1- | |
| | {[6-Furan-3-Yl-5-(3-Methyl-2h-Indazol-5-Yl)pyridin-3-Yl]oxy}-3-(1h-Indol-3-Yl)propan-2- | |
| | Amine\|Akt inhibitor VIII\|Inositol 1,3,4,5-tetrakisphosphate\|Lactoquinomycin\|N-[2-(4-amino-1,2,5- | |
| | oxadiazol-3-yl)-1-ethylimidazo[5,4-d]pyridin-7-yl]methyl]piperidin-4-amine\|4-[(2S)-2-(3- | |
| | Chlorophenyl)-2-hydroxyethyl]amino]-3-[7-methyl-5-(4-morpholinyl)-1H-benzimidazol-2-yl]-2(1H)- | |
| | pyridinone\|Bisindolylmaleimide IV\|Bisindolylmaleimide i\|(Z)-3-((1H-pyrrol-2-yl)methylene)indolin- | |
| | 2-one\|(S)-4-(2-(N-methylisoquinoline-5-sulfonamido)-3-oxo-3-(4-phenylpiperazin-1- | |
| | yl)propyl)phenyl isoquinoline-5-sulfonate\|Urea,(2,3-dihydro-2-oxo-3-(1-(1H-pyrrol-2- | |
| | yl)ethylidene)-1H-indol-5-yl)-\|4,5,6,7-Tetrabromobenzotriazole\|#N/A\|(2s)-1-(1h-Indol-3-Yl)-3-{[5- | |
| | (3-Methyl-1h-Indazol-5-Yl)pyridin-3-Yl]oxy}propan-2-Amine\|bisindolylmaleimide IX\|4,5,6- | |
| | Trihydroxy-3-methylphthalide\|Indiplon\|Myriocin\|Zaragozic acid A\|M2698 free base\|7-Chloro-3- | |
| | methyl-1-phenyl-1,2,4,5-tetrahydro-3-benzazepin-8-ol\|2-[2-[1-(3-Chlorophenyl)-3-fluoroazetidin- | |
| | 3-yl]ethynyl]pyridine\|Miransertib\|6-(2-chlorophenyl)-N′-(3-chlorophenyl)-7-(4-chlorophenyl)-N′- | |
| | methylpyrazolo[1,5-a]pyrimidine-3-carbohydrazide\|Afuresertib\|(R)-Lip-L-glu-L-ala- | |
| | OH\|Canertinib\|4-Hydroxybenzyl alcohol\|Capivasertib\|patasertib\|Enzastaurin\|Bms- | |
| | 754807\|(3R,5R,6E)-7-[4,5-bis(4-fluorophenyl)-2-(propan-2-yl)-1H-imidazol-1-yl]-3,5- | |
| | dihydroxyhept-6-enoic acid | |
| PK3CD | (2E)-2-[(4,5-dimethoxy-2-methyl-3,6-dioxocyclohexa-1,4-dien-1-yl)methylidene]undecanoic | Inhibitor\| |
| | acid\|5-(7-(methylsulfonyl)-2-morpholino-6,7-dihydro-5H-pyrrolo[2,3-d]pyrimidin-4-yl)pyrimidin-2- | Modulator |
| | amine\|2-((6-amino-9H-purin-9-yl)methyl)-5-methyl-3-o-tolylquinazolin-4(3H)-one\|(2S,3aS,7aS)-1- | |
| | [(2S)-2-[(2S)-1-ethoxy-1-oxopentan-2-yl]amino]propanoyl]-2,3,3a,4,5,6,7,7a-octahydroindole-2- | |
| | carboxylic acid;(2S)-2-amino-5-(diaminomethylideneamino)pentanoate\|2-(4-Morpholinyl)-4H-1- | |
| | benzopyran-4-one\|2-(Morpholin-4-yl)-benzo[h]chromen-4-one\|5-(2-((4-(Methylsulfonyl)piperazin- | |
| | 1-yl)methyl)-8-morpholinoimidazo[1,2-a]pyrazin-6-yl)pyrimidin-2-amine\|Wortmannin\|4-Phenyl-1- | |
| | (4-(tetrahydro-2-furyl)butyl)isonipecotic acid ethyl ester\|2,4-Diamino-6-{[(S)-[5-Chloro-8-Fluoro-4- | |
| | Oxo-3-(Pyridin-3-Yl)-3,4-Dihydroquinazolin-2-Yl](Cyclopropyl)methyl]amino}pyrimidine-5- | |
| | Carbonitrile\|Acalisib\|(S)-N-(1-(7-fluoro-2-(pyridin-2-yl)quinolin-3-yl)ethyl)-9H-purin-6-amine\|(E)-3- | |
| | (4-chlorophenyl)-1-[2-(3,5-diphenylpyrazol-1-yl)-4-methyl-thiazol-5-yl]prop-2-en-1-one\|(E)-2-[[3- | |
| | (3-methoxy-4-propargyloxyphenyl)-1-oxo-2-propenyl]amino]benzoic | |
| | acid\|Dezapelisib\|Tenalisib\|Mephenytoin\|Leniolisib\|Parsaclisib\|Buparlisib\|Umbralisib\|delalisib\|Du | |
| | velisib\|Copanlisib\|3-[4-amino-7-(2,3-dihydro-1H-inden-2-yl)pyrrolo[2,3-d]pyrimidin-5-yl]-5- | |
| | fluorophenol | |
| LYN | N-[4-[(4-ethylpiperazin-1-yl)methyl]-3-(trifluoromethyl)phenyl]-4-methyl-3-(1H-pyrrolo[2,3- | Inhibitor\| |
| | b]pyridin-4-yloxy)benzamide\|3-[4-(5-Hydroxy-2-methylanilino)pyrimidin-2- | Modulator |
| | yl]amino]benzenesulfonamide\|CID 73755245\|Bafetinib\|(R)-N2-{4-[(3- | |
| | chlorobenzyl)oxy]benzyl}alaninamide | |
| PTK7 | 2-(3-Fluoro-4-hydroxy-phenyl)-benzooxazol-6-ol | Inhibitor |
| DMPK | (E)-2-(2-(quinolin-2-yl)vinyl)phenol\|3-[4-(3-Chlorophenyl)-1-ethyl-7-methyl-2-oxo-1,2-dihydro-1,8- | .\|Inhibitor |
| | naphthyridin-3-yl]propanoic acid\|1-(3-Hydroxybenzyl)-3-(4-(pyridin-4-yl)thiazol-2-yl)urea\|Acetic | |
| | acid 2-heptylsulfanyl-phenyl ester | |
| ROS1 | (2R)-2-[5-[6-amino-5-[(1R)-1-[5-fluoro-2-(triazol-2-yl)phenyl]ethoxy]pyridin-3-yl]-4-methyl-1,3- | Inhibitor\| |
| | thiazol-2-yl]propane-1,2-diol\|3-[4-[(2S)-2-aminopropoxy]phenyl]-N-[(1R)-1-(3- | Modulator |
| | fluorophenyl)ethyl]imidazo[1,2-b]pyridazin-6-amine\|5-{3-[6-Methyl-4-(4-trifluoromethyl-phenyl)- | |
| | pyridin-2-yl]-phenyl}-pyridin-2-ylamine\|Lorlatinib\|3-[4-[(2S)-azetidin-2-yl]methoxy]phenyl]-N- | |
| | [(1R)-1-(3-fluorophenyl)ethyl]imidazo[1,2-b]pyridazin-6-amine\|(3R,4S,6R)-2-(hydroxymethyl)-6- | |
| | methoxyoxane-3,4,5-triol\|Repotrectinib\|3-[4-(2-aminopropoxy)phenyl]-N-[1-(3- | |
| | fluorophenyl)ethyl]imidazo[1,2-b]pyridazin-6-amine\|Entrectinib\|Crizotinib\|Axitinib | |
| PHKG2 | N-[(1S)-1-(5-fluoropyridin-2-yl)ethyl]-3-(3-propan-2-yloxy-1H-pyrazol-5-yl)imidazo[4,5-b]pyridin-5- | Inhibitor |
| | amine | |
| GRK5 | 5-(1-Piperidin-4-ylpyrazol-4-yl)-3-(6-pyrrolidin-1-yl-1,3-benzoxazol-2-yl)pyridin-2-amine\|4-[3- | Inhibitor |
| | [(2,6-difluorophenyl)methylcarbamoyl]-4-fluorophenyl]-N-(1H-indazol-5-yl)-6-methyl-2-oxo-3,4- | |
| | dihydro-1H-pyrimidine-5-carboxamide\|4-[3-[2-[(2,6-dimethylphenyl)methylamino]-2-oxoethyl]-4- | |
| | fluorophenyl]-N-(1H-indazol-5-yl)-6-methyl-2-oxo-3,4-dihydro-1H-pyrimidine-5-carboxamide\|CID | |
| | 118888905 | |
| WEE1 | 9-hydroxypyrrolo[3,4-c]carbazole-1,3(2H,6H)-dione\|Adavosertib\|CID 76900387 | Inhibitor |
| CDK2 | Roniciclib\|4-(2,6-dichlorobenzamido)-N-(piperidin-4-yl)-1H-pyrazole-3-carboxamide\|(2S)-2-[4- | Inhibitor\| |
| | (Acetylamino)phenyl]-N-(5-isopropyl-1,3-thiazol-2-yl)propanamide\|5-N- | Modulator |
| | Acetylardeemin\|Aristolactam AIIIa\|(7S)-2-(2-aminopyrimidin-4-yl)-7-(2-fluoroethyl)-1,5,6,7- | |
| | tetrahydro-4H-pyrrolo[3,2-c]pyridin-4-one\|2-fluoro-4-(4-(1-isopropyl-2-methyl-1H-imidazol-5- | |
| | yl)pyrimidin-2-ylamino)-N-methylbenzamide\|7-butyl-6-(4-methoxyphenyl)-5H-pyrrolo[2,3- | |
| | b]pyrazine\|4-(7-butyl-5H-pyrrolo[2,3-b]pyrazin-6-yl)phenol\|aminopurvalanol A\|Bms-265246\|N-[3- | |
| | [5-Iodo-4-[3-[(2-thienylcarbonyl)amino]propyl]amino]-2-pyrimidinyl]amino]phenyl]-1- | |
| | pyrrolidinecarboxamide\|N-[3-[[5-Bromo-4-[2-(1H-imidazol-5-YL)ethyl]amino]-2- | |
| | pyrimidinyl]amino]phenyl]-1-pyrrolidinecarboxamide\|Cdk1/2 Inhibitor II\|Cvt-313\|4-(6- | |
| | Cyclohexylmethoxy-9H-purin-2-ylamino)-N,N-diethylbenzamide\|Cdk4 Inhibitor\|1,4-dimethoxy- | |
| | 10H-acridine-9-thione\|Ryuvidine\|9H-Purin-6-amine,9-(1-methylethyl)-N-(phenylmethyl)-\|4-(2,4- | |
| | Dimethyl-1,3-thiazol-5-YL)pyrimidin-2-amine\|2-Amino-6-chloropyrazine\|6-(cyclohexylmethoxy)- | |
| | 9H-purin-2-amine\|1-Amino-6-cyclohex-3-enylmethyloxypurine\|N-(5-Cyclopropyl-1H-pyrazol-3- | |
| | YL)benzamide\|4-[3-Hydroxyanilino]-6,7-dimethoxyquinazoline\|Lysine Nz-Carboxylic | |
| | Acid\|Phenylaminoimidazo(1,2-alpha)pyridine\|8-Azapurine\|2-Anilino-6- | |
| | cyclohexylmethoxypurine\|5-Hydroxynaphthalene-1-sulfonamide\|N-(4-sulfamoylphenyl)-1H- | |
| | indazole-3-carboxamide\|4-[(6-Chloropyrazin-2-yl)amino]benzenesulfonamide\|N-phenyl-1H- | |
| | pyrazole-3-carboxamide\|6-Cyclohexylmethyloxy-2-(4′-hydroxyanilino)purine\|N-(3-Methylbut-2- | |
| | EN-1-YL)-9H-purin-6-amine\|L-cysteine-S-dioxide\|Bohemine\|6-Cyclohexylmethoxy-2-(3′- | |
| | chloroanilino)purine\|(S)-4-(2-(4-Amino-1,2,5-oxadiazol-3-yl)-1-ethyl-7-(piperidin-3-ylmethoxy)- | |
| | 1H-imidazo[4,5-c]pyridin-4-yl)-2-methylbut-3-yn-2-ol\|Hymenialdisine\|Alsterpaullone\|Indirubin-3′- | |
| | monoxime\|Indirubin-5-sulphonate\|SU9516\|Pyrazolopyridazine 1\|Pyrazolopyridazine 2\|4-[(2S)-2- | |
| | (3-Chlorophenyl)-2-hydroxyethyl]amino]-3-[7-methyl-5-(4-morpholinyl)-1H-benzimidazol-2-yl]- | |
| | 2(1H)-pyridinone\|6-(Cyclohexylmethoxy)pyrimidine-2,4,5-triamine\|06-Substituted Guanine Deriv. | |
| | 27\|06-Substituted Guanine Deriv. 32\|06-Substituted Guanine Deriv. 33\|4-[(7-oxo-6H- | |
| | pyrrolo[2,3-g][1,3]benzothiazol-8-ylidene)methylamino]-N-(2-pyridinyl)benzenesulfonamide\|6- | |
| | (Cyclohexylmethoxy)-5-nitrosopyrimidine-2,4-diamine\|Meriolin 8\|Meriolin 6\|4-(4-Propoxy-1h- | |
| | Pyrrolo[2,3-B]pyridin-3-Yl)pyrimidin-2-Amine\|Meriolin 4\|4-(4-Methoxy-1h-Pyrrolo[2,3-B]pyridin-3- | |
| | Yl)pyrimidin-2-Amine\|Meriolin 2\|4-(1H-Pyrrolo[2,3-B]pyridin-3-YL)pyrimidin-2-amine\|2-(4- | |
| | Methoxyphenyl)-3-phenylmaleimide\|06-Cyclohexylmethoxy-2-(4′-sulphamoylanilino) | |
| | purine\|#N/A\|N-(4-amino-5-cyano-6-phenylpyridin-2-yl)acetamide\|4-({5-Amino-1-[(2,6- | |
| | Difluorophenyl)carbonyl]-1h-1,2,4-Triazol-3-Yl}amino)benzenesulfonamide\|2-(1H-Indole-3-yl)-3- | |
| | phenylmaleimide\|1H-Pyrrole-2,5-dione,3,4-bis(4-methoxyphenyl)-\|3,4-diphenyl-1H-pyrrole-2,5- | |
| | dione\|purvalanol A\|3,4-Dihydro-6-[4-[3-(methylsulfonyl)phenyl]methyl]amino]-5- | |
| | (trifluoromethyl)-2-pyrimidinyl]amino]-2(1H)-quinolinone\|Rescovitine\|8-Bicyclo[2.2.1]hept-2-yl-2- | |
| | {4-[4-(3-hydroxypropyl)piperidin-1-yl]phenylamino}-8H-pyrido[2,3-d]pyrimidin-7-one\|2-({2-[(R)-2- | |
| | Amino-3-(4-hydroxy-2,6-dimethyl-phenyl)-propionyl]-1,2,3,4-tetrahydro-isoquinoline-3-carbonyl}- | |
| | amino)-propionic acid\|Purvalanol B\|(4-Amino-2-((1-(methylsulfonyl)piperidin-4- | |
| | yl)amino)pyrimidin-5-yl)(2,3-difluoro-6-methoxyphenyl)methanone\|ZK-Cdk\|Dinaciclib\|4-(1- | |
| | isopropyl-2-methyl-1H-imidazol-5-yl)-N-(4-(methylsulfonyl)phenyl)pyrimidin-2-amine\|Rgb- | |
| | 286638\|3-Pyridinemethanamine,5-(3-(4,6-difluoro-1H-benzimidazol-2-yl)-1H-indazol-5-yl)-N- | |
| | ethyl-4-methyl-\|N-(6,6-dimethyl-5-(1-methylpiperidine-4-carbonyl)-1,4,5,6-tetrahydropyrrolo[3,4- | |
| | c]pyrazol-3-yl)-3-methylbutanamide\|N-(5-(((5-(1,1-Dimethylethyl)-2-oxazolyl)methyl)thio)-2- | |
| | thiazolyl)-4-piperidinecarboxamide\|Fadraciclib\|N-(1-((2-(Dimethylamino)ethyl)amino)-2-methyl-1- | |
| | oxopropan-2-yl)-4-(4-(2-methyl-5-((2S,3R,4R,5S,6R)-3,4,5-trihydroxy-6-(methylthio)tetrahydro- | |
| | 2H-pyran-2-yl)benzyl)phenyl)-butanamide\|N-[4-[(4-methylpiperazin-1-yl)methyl]phenyl]-4-(7H- | |
| | pyrrolo[2,3-d]pyrimidin-4-ylamino)-1H-pyrazole-5-carboxamide\|Zotiraciclib\|Seliciclib\|1H-pyrrole- | |
| | 2,5-dione,3-(1-methyl-1h-indol-3-yl)-4-(1-methyl-6-nitro-1h-indol-3-yl)-\|Milciclib\|Olomoucine\|4- | |
| | (2,4-difluorophenyl)-5-fluoro-N-[3-(methylsulfonylmethyl)-5-(pentafluoro-lambda6- | |
| | sulfanyl)phenyl]pyrimidin-2-amine\|3-[5-Fluoro-4-[4-methyl-2-(methylamino)-1,3-thiazol-5- | |
| | yl]pyrimidin-2-yl]amino]benzenesulfonamide\|(Rac)-[ethyl(3-{[5-fluoro-4-(4-fluoro-2- | |
| | methoxyphenyl)pyrimidin-2-yl]-amino}benzyl)oxido-lambda6-sulfanylidene]cyanamide\|[3-[4- | |
| | (2,4-Difluorophenyl)-5-fluoropyrimidin-2-yl]amino]-5-(pentafluoro-lambda6- | |
| | sulfanyl)phenyl]methyl-methyl-oxo-lambda6-sulfanylidene]cyanamide\|ethyl (7S)-11-oxo-14-(2- | |
| | trimethylsilylethynyl)-2,4,10-triazatetracyclo[10.4.0.02,6.07,10]hexadeca-1(16),3,5,12,14- | |
| | pentaene-5-carboxylate\|(rac)-4-{4-Fluoro-2-[(3-fluorobenzyl)oxy]phenyl}-N-{3-[(S- | |
| | methylsulfonimidoyl)methyl]phenyl}-1,3,5-triazin-2-amine\|U.S. Pat No. 9,670,161,1 5-Fluoro-4-(4-fluoro-2- | |
| | methoxyphenyl)-N-{4-[(methylsulfonyl)methyl]pyridin-2-yl}pyridin-2-amin,\|3-[4-(2- | |
| | Methoxyphenyl)-2-pyridinyl]amino]-benzenemethanesulfonamide\|6-(4-fluoro-2-methoxyphenyl)- | |
| | N-[4-[(methylsulfonimidoyl)methyl]-6-(trifluoromethyl)pyridin-2-yl]pyrimidin-4-amine\|2-[(3- | |
| | Hydroxyphenyl)amino]-4-[4-Methyl-2-(Methylamino)-1,3-Thiazol-5-Yl]pyrimidine-5-Carbonitrile\|8- | |
| | Methyl-2-morpholin-4-yl-7-phenoxychromen-4-one\|N-hydroxy-4-[(4-imidazo[1,2-a]pyridin-3- | |
| | ylpyrimidin-2-yl)amino]benzamide\|Tyr-Pro-Phe-Phe-OC(CH3)3\|(1-((1-(4-Bromobenzyl)-1H- | |
| | imidazol-5-yl)methyl)-4-(naphthalen-1-yl)-1H-pyrrol-3-yl)(morpholino)methanone\|(rac)-5-Fluoro- | |
| | 4-(4-fluoro-2-methoxyphenyl)-N-{6-methoxy-4-[(S-methylsulfonimidoyl)methyl]pyridin-2- | |
| | yl}pyridin-2-amine\|Cpzen-45\|2-(4-(bis(2-chloroethyl)amino)phenyl)-N-(5-((5-tert-butyloxazol-2- | |
| | yl)methylthio)thiazol-2-yl)acetamide\|N-hydroxy-5-[4-(2-methyl-3-propan-2-ylimidazol-4- | |
| | yl)pyrimidin-2-yl]amino]thiophene-2-carboxamide\|2-Amino-5-phosphonopentanoic acid\|7- | |
| | cyclopentyl-2-[5-(4-isopropyl-piperazin-1-yl)-pyridin-2-ylamino]-7H-pyrrolo[2,3-d]pyrimidine-6- | |
| | carboxylic acid dimethylamide\|4-[4-[2-[5-[(5-tert-butyl-1,3-oxazol-2-yl)methylsulfanyl]-1,3-thiazol- | |
| | 2-yl]amino]-2-oxoethyl]phenoxy]-N-hydroxybutanamide\|2-N-(4-aminocyclohexyl)-9-cyclopentyl- | |
| | 6-N-[6-(furan-2-yl)pyridin-3-yl]methyl]purine-2,6-diamine\|2-[(E)-2-(2,6-dichlorophenyl)ethenyl]- | |
| | 5,7-dihydroxy-8-(3-hydroxy-1-methylpiperidin-4-yl)chromen-4-one\|4-[(5-Piperazin-1-ylpyridin-2- | |
| | yl)amino]spiro[1,3,5,11-tetrazatricyclo[7.4.0.02,7]trideca-2,4,6,8-tetraene-13,1′-cyclohexane]-10- | |
| | one\|CID 73891136\|2-N-(4-piperazin-1-ylpyridin-3-yl)-4-N-pyridin-3-yl-1,3-thiazole-2,4- | |
| | dicarboxamide\|Fascaplysin\|N-[(2-imidazol-1-ylphenyl)methyl]-2-(1-methylpiperidin-4-yl)oxy-8- | |
| | propan-2-ylpyrazolo[1,5-a][1,3,5]triazin-4-amine | |
| EPHA4 | 1H-Imidazo[2,1-f]purine-2,4(3H,8H)-dione,7-(5-hydroxy-2-methylphenyl)-8-(2-methoxyphenyl)- | Inhibitor\|. |
| | 1-methyl-\|(2S)-2-[(4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid\|4-(4-Chlorobenzylthio)-2-aminobutanoic acid | |
| LIMK1 | 1-(3-Hydroxybenzyl)-3-(4-(pyridin-4-yl)thiazol-2-yl)urea\|(S)-N-(3-bromophenyl)-N′-cyano-2- | Inhibitor |
| | methyl-4-(5-methyl-7H-pyrrolo[2,3-d]pyrimidin-4-yl)piperazine-1-carboximidamide\|1-[5-[6-(2,6- | |
| | Dimethyl-4-pentan-3-yloxyphenyl)-2-pyrazin-2-ylpyrimidin-4-yl]-1,3-thiazol-2-yl]-3-methylurea\|1- | |
| | Ethyl-3-[5-[6-(4-methoxy-2,6-dimethylphenyl)-2-pyrazin-2-ylpyrimidin-4-yl]-1,3-thiazol-2- | |
| | yl]urea\|N-[5-[6-(2,6-dichloro-4-methoxyphenyl)-2-pyrazin-2-ylpyrimidin-4-yl]-1,3-thiazol-2- | |
| | yl]acetamide | |
| MK07 | 2-{[2-ethoxy-4-(4-hydroxypiperidin-1-yl)phenyl]amino}-5,11-dimethyl-5,11-dihydro-6H- | Inhibitor |
| | pyrimido[4,5-b][1,4]benzodiazepin-6-one\|2-[2-ethoxy-4-[(3S)-3-[(1-methylpiperidin-4- | |
| | yl)amino]pyrrolidine-1-carbonyl]anilino]-5,11-dimethylpyrido[4,3-b][1,4]benzodiazepin-6-one\|2-[2- | |
| | Ethoxy-4-(4-pyrrolidin-1-ylpiperidine-1-carbonyl)anilino]-5,11-dimethylpyrido[4,3- | |
| | b][1,4]benzodiazepin-6-one\|2-[2-Ethoxy-4-[4-(4-methyl-1,4-diazepan-1-yl)piperidine-1- | |
| | carbonyl]anilino]-5,11-dimethylpyrido[4,3-b][1,4]benzodiazepin-6-one | |
| GSK3B | Kenpaullone\|2-(2-chloro-4-iodophenylamino)-N-cyclopropylmethoxy-3,4-difluorobenzamide\|3-(5- | Inhibitor\|.\| |
| | fluorobenzofuran-3-yl)-4-(5-methyl-5H-[1,3]dioxolo[4,5-f]indol-7-yl)-1H-pyrrole-2,5- | Modulator |
| | dione\|Felodipine\|3-(2-Phenyl-2-(piperazin-1-yl)ethyl)phenol\|3-[2-(4-fluorophenyl)ethylamino]-1- | |
| | methyl-4-(2-methyl-1H-indol-3-yl)pyrrole-2,5-dione\|GSK-3 Inhibitor XIN-(5-methyl-1H-pyrazol-3- | |
| | yl)-2-phenylquinazolin-4-amine\|GSK-3 Inhibitor II\|GSK-3beta Inhibitor XI\|(7S)-2-(2- | |
| | aminopyrimidin-4-yl)-7-(2-fluoroethyl)-1,5,6,7-tetrahydro-4H-pyrrolo[3,2-c]pyridin-4- | |
| | one\|Alsterpaullone,2-Cyanoethyl\|N-[3-[5-Iodo-4-[3-[(2-thienylcarbonyl)amino]propyl]amino]-2- | |
| | pyrimidinyl]amino]phenyl]-1-pyrrolidinecarboxamide\|N-[3-[5-Bromo-4-[2-(1H-imidazol-5- | |
| | YL)ethyl]amino]-2-pyrimidinyl]amino]phenyl]-1-pyrrolidinecarboxamide\|Cdk/Crk Inhibitor\|1H- | |
| | Pyrazolo[3,4-b]quinoxalin-3-amine\|6-N-[2-[4-(2,4-dichlorophenyl)-5-imidazol-1-ylpyrimidin-2- | |
| | yl]amino]ethyl]-3-nitropyridine-2,6-diamine\|2-Chloro-5-[4-(3-chloro-phenyl)-2,5-dioxo-2,5- | |
| | dihydro-1H-pyrrol-3-ylamino]-benzoic acid\|Phosphoaminophosphonic acid-adenylate | |
| | ester\|Pyrazolopyridazine 1\|Pyrazolopyridazine 2\|3-(3-chloro-4-hydroxyphenylamino)-4-(4- | |
| | nitrophenyl)-1H-pyrrole-2,5-dione\|Bisindolylmaleimide i\|1-Azakenpaullone\|Thieno analogue of | |
| | kenpaullone\|Manzamine A\|12,13-dehydro-8-O-acetylmanzamine A\|12,13-dehydromanzamine | |
| | A\|8-O-(4-bromobenzenesulfonyl)manzamine F\|8-O-(4-chlorobenzenesulfonyl)manzamine F\|CID | |
| | 6474830\|6-Deoxymanzamine X\|9-N-ethyl-8-ethoxy-manzamine A\|9-N-methyl-8-methoxy- | |
| | manzamine A\|8-O-(4-toluenesulfonyl)manzamine A\|8-Hydroxymanzamine A\|1,3-Benzothiazol-2- | |
| | yl(2-((2-(3-pyridinyl)ethyl)amino)-4-pyrimidinyl)acetonitrile\|2-(Phenylamino)-3- | |
| | phenylmaleimide\|4,5,6,7-Tetrabromobenzotriazole\|2-chloro-5-(2-phenyl-5-(pyridin-4-yl)-1H- | |
| | imidazol-4-yl)phenol\|06-Cyclohexylmethoxy-2-(4′-sulphamoylanilino) | |
| | purine\|#N/A\|bisindolylmaleimide IX\|Ellagic acid\|N-(8-(3-cyanophenyl)-9H-purin-6- | |
| | yl)pentanamide\|3-(6-(phenylamino)-9H-purin-8-yl)benzonitrile\|N,8-diphenyl-9H-purin-6-amine\|6- | |
| | hydroxymanzamine A\|neo-Kauluamine\|3,4-Dihydro-6-[4-[3- | |
| | (methylsulfonyl)phenyl]methyl]amino]-5-(trifluoromethyl)-2-pyrimidinyl]amino]-2(1H)- | |
| | quinolinone\|N-(6-chloro-1H-indazol-3-yl)butanamide\|N-[6-(4-Hydroxyphenyl)-1h-Indazol-3- | |
| | Yl]butanamide\|N-(6-(4-aminophenyl)-1H-indazol-3-yl)butyramide\|N-(6-(3-hydroxyphenyl)-1H- | |
| | indazol-3-yl)butyramide\|N-[6-(trifluoromethyl)-1H-indazol-3-yl]butanamide\|N-[6-bromo-1H- | |
| | indazol-3-yl]butanamide\|N-(6-(furan-3-yl)-1H-indazol-3-yl)butyramide\|N-(6-(pyridin-4-yl)-1H- | |
| | indazol-3-yl)butyramide\|1,2-Ethanediamine,N-(4-(2,4-dichlorophenyl)-5-(1H-imidazol-2-yl)-2- | |
| | pyrimidinyl)-N′-(5-nitro-2-pyridinyl)-\|N-(6-(thiophen-3-yl)-1H-indazol-3-yl)butyramide\|N-(6-phenyl- | |
| | 1H-indazol-3-yl)butyramide\|N-(6-(2-chlorophenyl)-1H-indazol-3-yl)butyramide\|(E)-N-(6-(prop-1- | |
| | enyl)-1H-indazol-3-yl)butyramide\|N-(6-chloro-5-phenyl-1H-indazol-3-yl)butanamide\|N-(6-chloro- | |
| | 5-p-tolyl-1H-indazol-3-yl)butyramide\|N-(6-benzyl-1H-indazol-3-yl)butyramide\|N-(6-(4- | |
| | fluorophenyl)-1H-indazol-3-yl)butyramide\|N-(6-phenethyl-1H-indazol-3-yl)butyramide\|N-(6- | |
| | (pyridin-3-yl)-1H-indazol-3-yl)butyramide\|Paullone\|N2-(2-((4-(2,4-Dichlorophenyl)-5-(1H- | |
| | imidazol-2-yl)pyrimidin-2-yl)amino)ethyl)-5-nitropyridine-2,6-diamine\|leucettamine B\|4-(5-bromo- | |
| | 1H-indol-3-yl)pyrimidin-2-amine\|Indiplon\|3-[6-(3-Aminophenyl)-7H-pyrrolo[2,3-d]pyrimidin-4- | |
| | yl]oxy]phenol\|D-Phenylalanyl-N-(3-Fluorobenzyl)-L-Prolinamide\|2-hydroxy-3-(5- | |
| | (morpholinomethyl)pyridin-2-yl)-1H-indole-5-carbonitrile\|4-Benzyl-2-methyl-1,2,4-thiadiazolidine- | |
| | 3,5-dione\|6-((2-((4-(2,4-Dichlorophenyl)-5-(4-methyl-1H-imidazol-2-yl)pyrimidin-2- | |
| | yl)amino)ethyl)amino)nicotinonitrile\|N-(4-Methoxybenzyl)-N′-(5-nitro-1,3-thiazol-2-YL)urea\|[1-(3- | |
| | Fluoroanilino)-5-methylisoquinolin-4-yl]-morpholin-4-ylmethanone\|Tideglusib\|3-(9-Fluoro-2- | |
| | (piperidine-1-carbonyl)-1,2,3,4-tetrahydro-[1,4]diazepino[6,7,1-hi]indol-7-yl)-4-(imidazo[1,2- | |
| | alpyridin-3-yl)-1H-pyrrole-2,5-dione\|Lithium\|(2S)-N-[(2R)-1-[2-[(2S)-1-[(2-amino-2- | |
| | oxoethyl)amino]-4-methyl-1-oxopentan-2-yl]amino]-2-oxoethyl]amino]-1-oxo-3-sulfanylpropan-2- | |
| | yl]-2-[(2S,3R)-2-[(2S,3S)-2-[(2S)-2-[[(2R)-2-amino-3-sulfanylidenepropanoyl]amino]-3-(4- | |
| | hydroxyphenyl)propanoyl]amino]-3-methylpentanoyl]amino]-3- | |
| | hydroxybutanoyl]amino]butanediamide\|(S)-3-(8-(Dimethylaminomethyl)-6,7,8,9- | |
| | tetrahydropyrido(1,2-a)indol-10-yl)-4-(1-methyl-3-indolyl)-1H-pyrrole-2,5-dione hydrochloride\|3- | |
| | (2-aminopyrimidin-4-yl)-5-chloro-1H-indol-2-ol | |
| ANPRA | 7-Methoxy-4-((6-phenyl-[1,2,4]triazolo[4,3-b]pyridazin-3-yl)methoxy)quinoline\|Ataciguat\|(Hept- | Agonist\| |
| | cyclo(Cys-His-Phe-D-Ala-Gly-Arg-D-nle-Asp-Arg-Ile-Ser-Cys)-Tyr-(Arg mimetic)- | Activator\| |
| | NH2)\|Ularitide\|BNP-32 (human) | Modulator\| |
| | | Binder |
| AKT3 | Levosimendan\|(2s)-1-{[6-Furan-3-Yl-5-(3-Methyl-2h-Indazol-5-Yl)pyridin-3-Yl]oxy}-3-(1h-Indol-3- | Inhibitor\| |
| | Yl)propan-2-Amine\|Akt inhibitor VIII\|20-Amino-14-fluoro-4,17-dimethyl-7,10,18-trioxa-4,5,21- | Modulator\|. |
| | triazatetracyclo[17.3.1.02,6.011,16]tricosa-1(23),2,5,11(16),12,14,19,21-octaene-3- | |
| | carbonitrile\|Diethyl 6-methoxy-5,7-dihydroindolo[2,3-b]carbazole-2,10-dicarboxylate\|Triciribine | |
| | phosphate\|(S)-4-(2-(4-Amino-1,2,5-oxadiazol-3-yl)-1-ethyl-7-(piperidin-3-ylmethoxy)-1H- | |
| | imidazo[4,5-c]pyridin-4-yl)-2-methylbut-3-yn-2-ol\|2-(3-(7-Hydroxy-4,5-dihydropyrazolo[1,5- | |
| | a]quinolin-3-yl)propanamido)benzoic acid\|2-(4-(1-Aminocyclobutyl)phenyl)-3-phenylimidazo[1,2- | |
| | b]pyridazine-6-carboxamide\|1-Butanone,1-(3-(4-(3-bromo-1H-pyrazolo(3,4-d)pyrimidin-4-yl)-1- | |
| | piperazinyl)-4-methyl-5-((2-(1-pyrrolidinyl)ethyl)amino)phenyl)-4,4,4-trifluoro-\|M2698 free | |
| | base\|Miransertib\|Uprosertib\|MK-2206\|Capivasertib\|2-(1H-pyrazol-3-yl)-1H-benzimidazole\|Tak- | |
| | 285\|Plomestane\|Argiotoxin 636\|1-Methylnicotinamide\|3-(((3-(4-((Methylsulfonyl)amino)phenyl)-4- | |
| | oxo-4H-chromen-7-yl)oxy)methyl)benzoic acid\|Azatadine\|2-Phenyl-6-(propan-2-yl)-4H-1- | |
| | benzopyran-4-one\|Sulfamic acid 16-sulfamoyloxy-hexadecyl ester\|Empagliflozin\|3-(5-Bromo-6- | |
| | methoxy-2-naphthyl)pyridine\|Phenylcarbamic Acid Biphenyl-3-yl Ester\|4- | |
| | (Cyclohexylamino)pyrimidine-2-carbonitrile\|isoPPADS\|(E)-N-hydroxy-3-[4-[(E)-(2,3,4,5,6- | |
| | pentafluorophenyl)methoxyiminomethyl]phenyl]prop-2-enamide\|Pegcantratinib\|Ethyl 3-(3- | |
| | bromophenyl)-5,7-dihydroxy-4-oxo-chromene-2-carboxylate\|6-[(3-chloro-4-fluoro-phenyl)methyl]- | |
| | 4-hydroxy-N,N,2-trimethyl-3,5-dioxo-7,8-dihydro-2,6-naphthyridine-1-carboxamide\|N-[2-(4- | |
| | amino-1,2,5-oxadiazol-3-yl)-1-ethylimidazo[5,4-d]pyridin-7-yl]methyl]piperidin-4-amine | |
| M4K4 | 2-N-(3,5-dichlorophenyl)-4-N-[4-(dimethylamino)cyclohexyl]-5-(3-methyl-1,2-oxazol-5- | Inhibitor |
| | yl)pyrimidine-2,4-diamine\|6-(3-Fluorophenyl)pyrido[3,2-d]pyrimidin-4-amine\|6-Phenyl quinazolin- | |
| | 4-amine\|6-(2-Methoxyphenyl)quinazolin-4-amine\|6-(3-Methoxyphenyl)quinazolin-4-amine | |
| PLK4 | 2-{[2-ethoxy-4-(4-hydroxypiperidin-1-yl)phenyl]amino}-5,11-dimethyl-5,11-dihydro-6H- | Inhibitor |
| | pyrimido[4,5-b][1,4]benzodiazepin-6-one\|CFI-400945 free base | |
| TNIK | 4-Methoxy-3-[2-[3-methoxy-4-(4-methylpiperazin-1-yl)anilino]pyridin-4-yl]benzonitrile | Inhibitor |
| EPHB6 | (2S)-2-[[(4R)-4-[(3R,5R,8R,9S,10S,13R,14S,17R)-3-hydroxy-10,13-dimethyl- | Inhibitor |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid | |
| PTK6 | [2-chloro-4-[6-cyclopropyl-3-(1H-pyrazol-4-yl)imidazo[1,2-a]pyrazin-8-yl]amino]phenyl]-(2,2- | Inhibitor |
| | dimethylpiperazin-1-yl)methanone](+/−)-2-(2-Cinnamamidobenzamido)-3-phenylpropanoic acid | |
| CLK2 | (Z)-1-(3-Ethyl-5-methoxy-2,3-dihydrobenzothiazol-2-ylidene)propan-2-one\|5- | Inhibitor |
| | (benzo[d][1,3]dioxol-5-yl)-N-(3,5-dichlorobenzyl)pyrimidin-4-amine\|4-(1,3-benzodioxol-5-yl)-N- | |
| | [(3,5-dichlorophenyl)methyl]pyrimidin-2-amine | |
| EPHB2 | 1H-Imidazo[2,1-f]purine-2,4(3H,8H)-dione,7-(5-hydroxy-2-methylphenyl)-8-(2-methoxyphenyl)- | Inhibitor |
| | 1-methyl-\|(2S)-2-[(4R)-4-[(3R,5R,8R,9S,10S,13R,14S,17R)-3-hydroxy-10,13-dimethyl- | |
| | 2,3,4,5,6,7,8,9,11,12,14,15,16,17-tetradecahydro-1H-cyclopenta[a]phenanthren-17- | |
| | yl]pentanoyl]amino]-3-(1H-indol-3-yl)propanoic acid\|Phosphoaminophosphonic acid-adenylate | |
| | ester\|Snewiqprlpqh\|6-benzyl-3-(pyridin-4-yl)-5,6-dihydro-1H-pyrazolo[4,3-g]quinazolin-7(8H)- | |
| | one\|(3S)-3-methylsulfanyl-1-[2-[4-[4-(1-methyl-1,2,4-triazol-3-yl)phenyl]-3,6-dihydro-2H-pyridin- | |
| | 1-yl]-2-oxoethyl]-N-[3-(6-propan-2-yloxypyridin-3-yl)-1H-indazol-5-yl]pyrrolidine-3- | |
| | carboxamide\|Ulixertinib\|Pirtobrutinib | |
| BLK | N-(2-chloro-6-methylphenyl)-2-[(2,6-dimethylpyrimidin-4-yl)amino]-1,3-thiazole-5- | Inhibitor |
| | carboxamide\|Blk-IN-1\|2-methyl-N-[2-({3-[2-(prop-2-enamido)acetamido]phenyl}amino)pyrimidin- | |
| | 5-yl]-5-[3-(trifluoromethyl)benzamido]benzamide\|(5-(3-methoxyphenyl)-1-pentyl-1H-pyrrol-3- | |
| | yl)(naphthalen-1-yl)methanone | |
| FLT3 | N2-(1H-Indol-5-yl)-6-(pyridin-4-yl)pyrazine-2,3-diamine\|4-cyano-N-(5-(1-(2- | Inhibitor\|.\| |
| | (dimethylamino)acetyl)piperidin-4-yl)-2′,3′,4′,5′-tetrahydro-[1,1′-biphenyl]-2-yl)-1H-imidazole-2- | Modulator\| |
| | carboxamide\|3-(1H-indol-5-yl)-5-(4-((4-methylpiperazin-1-yl)methyl)phenyl)-1H-pyrrolo[2,3- | Binder |
| | b]pyridine\|Flt-3 inhibitor\|Flt-3 Inhibitor II\|(5-Phenyl-thiazol-2-yl)-(4-(2-pyrrolidin-1-yl-ethoxy)- | |
| | phenyl)-amine\|8-Bromo-2-((1-methylpiperidin-4-yl)amino)-4-((4- | |
| | phenoxyphenyl)amino)pyrido[4,3-D]pyrimidin-5(6H)-one\|3-Phenyl-1H-benzofuro[3,2- | |
| | c]pyrazole\|3-[(4-chlorophenyl)-(1H-imidazol-2-yl)methylidene]-5-[(1-ethylpiperidin-4-yl)amino]- | |
| | 1H-indol-2-one\|Benzo[c]-2,6-naphthyridin-5-amine,N-(2-chlorophenyl)-7-(1H-1,2,4-triazol-5-yl)- | |
| | \|1-(4-((4-Ethylpiperazin-1-yl)methyl)-3-(trifluoromethyl)phenyl)-3-(4-((6-(methylamino)pyrimidin- | |
| | 4-yl)oxy)phenyl)urea\|N-[(1S)-1-(5-fluoropyridin-2-yl)ethyl]-3-(3-propan-2-yloxy-1H-pyrazol-5- | |
| | yl)imidazo[4,5-b]pyridin-5-amine\|Bis(benzo[b]furan-2-yl)methanone\|(benzo[b]furan-2-yl)-(1H- | |
| | indol-2-yl)methanone\|Bis(5-aminobenzo[b]furan-2-yl)methanone\|Bis(5-acetoxybenzo[b]furan-2- | |
| | yl)methanone\|Bis(6-hydroxybenzo[b]furan-2-yl)methanone\|Bis(5-hydroxybenzo[b]furan-2- | |
| | yl)methanone\|4-(4-aminophenyl)-1H-indazol-3-amine\|N-[4-(3-amino-1H-indazol-4- | |
| | yl)phenyl]benzamide\|(5-fluoro-1H-indol-2-yl)-(1H-indol-2-yl)methanone\|Bis(1H-2- | |
| | indolyl)methanone 12\|(1H-indol-2-yl)(5-phenoxy-1H-indol-2-yl)methanone\|Bis(1H-2- | |
| | indolyl)methanone 11\|(5-hydroxy-1H-indol-2-yl)-(1H-indol-2-yl)methanone\|di(1H-indol-2- | |
| | yl)methanone\|2-(3,4-Dimethoxybenzamido)thiophene-3-carboxamide\|(Z)-5-Chloro-3-((3,5- | |
| | dimethyl-1H-pyrrol-2-yl)methylene)indolin-2-one\|L-Tryptophanamide,N-acetyl-D-tyrosyl-(4R)-4- | |
| | hydroxy-L-prolyl-L-asparaginyl-L-threonyl-L-phenylalanyl-2-azaglycyl-L-leucyl-N5- | |
| | (imino(methylamino)methyl)-L-ornithyl-\|Tandutinib\|5-oxo-1-tosyl-2,5-dihydro-1Hpyrrol-2-yl | |
| | acetate\|4-Pyridin-2-yl-5-quinolin-2-yl-thiazol-2-ylamine\|Urea,N-(2-methoxy-5-methylphenyl)-N′- | |
| | (6-((6-methoxy-7-(3-(4-methyl-1-piperazinyl)propoxy)-4-quinazolinyl)amino)-2-benzothiazolyl)- | |
| | \|(E)-(4-(2-(1H-indazol-3-yl)vinyl)phenyl)(piperazin-1-yl)methanone\|3-(1H-indol-5-yl)-5-(3,4,5- | |
| | trimethoxyphenyl)-1H-pyrrolo[2,3-b]pyridine\|1-[(Trans-4-Aminocyclohexyl)methyl]-N-Butyl-3-(4- | |
| | Fluorophenyl)-1h-Pyrazolo[3,4-D]pyrimidin-6-Amine\|Unii-uch3qqh1C0\|N-[4-[(4-methylpiperazin- | |
| | 1-yl)methyl]phenyl]-4-(7H-pyrrolo[2,3-d]pyrimidin-4-ylamino)-1H-pyrazole-5- | |
| | carboxamide\|2MT30Ehi63\|MK-2461\|(E)-5-(3-Fluorostyryl)isatin\|(S,E)-N-(1-((5-(2-((4- | |
| | Cyanophenyl)amino)-4-(propylamino)pyrimidin-5-yl)pent-4-yn-1-yl)amino)-1-oxopropan-2-yl)-4- | |
| | (dimethylamino)-N-methylbut-2-enamide\|1(2H)-Isoquinolinone,5,8-dichloro-2-((1,2-dihydro-4- | |
| | methoxy-6-methyl-2-oxo-3-pyridinyl)methyl)-3,4-dihydro-7-((R)-methoxy-3-oxetanylmethyl)-\|2-(1- | |
| | (Aminomethyl)-3-butylcyclopentyl)acetic acid\|(3R,4R)-4-Amino-1-[4-[(3- | |
| | methoxyphenyl)amino]pyrrolo[2,1-f][1,2,4]triazin-5-yl]methyl]piperidin-3-ol\|Tiludronic | |
| | Acid\|Famitinib\|Mivavotinib\|Pacritinib\|Quizartinib\|Pexidartinib\|Midostaurin\|Zidovudine\|Nintedanib\| | |
| | Lestaurtinib\|Ponatinib\|Gilteritinib\|6,7-Dimethyl-2-phenylquinoxaline\|H-DL-Tyr(2,6-diMe)-DL-Tic- | |
| | DL-Lys(Cbz)-NHPh\|Axitinib | |
| CDK13 | (R,E)-N-(4-(3-((5-chloro-4-(1H-indol-3-yl)pyrimidin-2-yl)amino)piperidine-1-carbonyl)phenyl)-4- | Inhibitor |
| | (dimethylamino)but-2-enamide | |
| TTK | [4-[[4-(Ethylamino)-5-(trifluoromethyl)pyrimidin-2-yl]amino]-2-fluoro-5-methoxyphenyl]-morpholin- | Inhibitor |
| | 4-ylmethanone\|1,9-Pyrazoloanthrone\|Empesertib\|N2-(2-Ethoxy-4-(4-methyl-4H-1,2,4-triazol-3- | |
| | yl)phenyl)-6-methyl-N8-neopentylpyrido[3,4-d]pyrimidine-2,8-diamine\|N-cyclopropyl-4-[7-[(3- | |
| | hydroxy-3-methylcyclobutyl)methylamino]-5-pyridin-3-yloxypyrazolo[1,5-a]pyrimidin-3-yl]-2- | |
| | methylbenzamide\|c[RGDf-(S,R)-alpha-Dfm-F]\|8-[4-(4-Methylpiperazin-1-yl)phenoxy]-4,5-dihydro- | |
| | [1,2]oxazolo[4,3-h]quinazoline-3-carboxamide\|2′-Fluor-uridine | |
| MTOR | 2-PyridinecarboxaMide,N-(5-fluoro-2-pyridinyl)-4-[(5-fluoro-3-pyridinyl)oxy]-6-Methyl-\|2-{2- | Inhibitor\| |
| | Azaniumyl-6-[(4-nitrophenoxycarbonyl)amino]hexanamido}-6-({[(4- | Modulator |
| | nitrophenyl)methoxy]carbonyl}amino)hexanoate\|Retinal\|Phenyl (2S,3S)-3-(4-chlorophenyl)-8- | |
| | methyl-8-azabicyclo[3.2.1]octane-2-carboxylate\|7-Fluoro-2-(4-methoxyphenyl)chroman-4-one\|3- | |
| | benzyl-5-fluorobenzo[d]oxazol-2(3H)-one\|2-(4-Cyclohexylthiosemicarbazono)methyl-phenol\|4- | |
| | {4-[2-(Phenyl-2-pyridinylamino)ethoxy]phenyl}butyric Acid\|Apnea\|Quinoline,6-(1-methyl-1- | |
| | (methylsulfonyl)ethyl)-8-(3-((1E)-2-(3-methyl-1,2,4-oxadiazol-5-yl)-2-(4- | |
| | (methylsulfonyl)phenyl)ethenyl)phenyl)-\|4-(((2S,3R,4S,5S,6R)-3,4,5-Trihydroxy-6- | |
| | (hydroxymethyl)tetrahydro-2H-pyran-2-yl)oxy)benzaldehyde\|6-Chloro-4-methyl-[1,2]oxazolo[5,4- | |
| | b]pyridin-3-amine\|4-[8-amino-1-(7-chloro-1H-indol-2-yl)imidazo[1,5-a]pyrazin-3-yl]cyclohexane- | |
| | 1-carboxylic acid\|2-Piperazinyl-8-phenyl-4H-1-benzopyran-4-one\|Torin 1\|1-cyclopentyl-3-(1H- | |
| | pyrrolo[2,3-b]pyridin-5-yl)-1H-pyrazolo[3,4-d]pyrimidin-4-amine\|2-(4-amino-1-isopropyl-1H- | |
| | pyrazolo[3,4-d]pyrimidin-3-yl)-1H-indol-5-ol\|Tyr-D-Phe-Gly-Trp-NMeNle-Asp-Phe-NH2\|2- | |
| | morpholino-4H-pyrimido[2,1-a]isoquinolin-4-one\|2-(Morpholin-4-yl)-benzo[h]chromen-4-one\|2-(2- | |
| | Methyl-morpholin-4-yl)-benzo[h]chromen-4-one\|C-16-(S)-7-methylindolerapamycin\|C-16-(S)-3- | |
| | methylindolerapamycin\|4-(6-morpholino-9H-purin-2-yl)phenol\|(4-(6-morpholino-9H-purin-2- | |
| | yl)phenyl)methanol\|4-(2-(1H-indol-6-yl)-9H-purin-6-yl)morpholine\|4-(2-(thiophen-3-yl)-9H-purin-6- | |
| | yl)morpholine\|3-(6-morpholino-9H-purin-2-yl)phenol\|2-(6-morpholino-9H-purin-2-yl)phenol\|4-(2- | |
| | (thiophen-2-yl)-9H-purin-6-yl)morpholine\|2-chloro-N-(6-cyanopyridin-3-yl)propanamide\|Torin 2\|3- | |
| | Dimethylaminomethyl-1H-indol-4-ol\|Rapamycin prodrug\|[5-[2,4-Bis((3S)-3-methylmorpholin-4- | |
| | yl)pyrido[2,3-d]pyrimidin-7-yl]-2-methoxyphenyl]methanol\|Trombodipine\|Chlorocyclinone C\|Gdc- | |
| | 0349\|5-(9-Isopropyl-8-methyl-2-morpholino-9H-purin-6-yl)pyrimidin-2-amine\|Cortisone | |
| | acetate\|N-(4-(4-(2-(difluoromethyl)-4-methoxy-1H-benzo[d]imidazol-1-yl)-6-morpholino-1,3,5- | |
| | triazin-2-yl)phenyl)-2-(dimethylamino)ethanesulfonamide\|4-(Furan-2-yl)thieno(3,2-d)pyrimidin-2- | |
| | amine\|2H-1-Benzopyran-7-ol,3,4-dihydro-3,4-bis(4-hydroxyphenyl)-8-methyl-,(3R,4S)-\|Cc- | |
| | 223\|BGT-226 free base\|Bimiralisib\|Osi- | |
| | 027\|Sapanisertib\|Vistusertib\|Apitolisib\|Salirasib\|Voxtalisib\|Gedatolisib\|Samotolisib\|PF- | |
| | 04691502\|Dactolisib\|N-(4-Chloro-3-((3-methyl-2-butenyl)oxy)phenyl)-2-methyl-3- | |
| | furancarbothioamide\|CID 66577114\|2-Thiomethyl-3-phenylpropanoic | |
| | acid\|Ridaforolimus\|Temsirolimus\|Everolimus\|Sirolimus\|Zotarolimus\|Novolimus\|Glasdegib\|4- | |
| | ((1R,2R)-2-Hydroxycyclohexyl)-2(trifluoromethyl)benzonitrile\|4-[3-Hydroxyanilino]-6,7- | |
| | dimethoxyquinazoline\|N-(4-(Diethylamino)benzyl)-4-methoxy-N-(p- | |
| | tolyl)benzenesulfonamide\|(2,5-Dimethoxybenzyl)(2-phenylethyl)amine\|N-(9-chloro-2-(furan-2-yl)- | |
| | [1,2,4]triazolo[1,5-c]quinazolin-5-yl)-2-phenylacetamide | |
| YES | Unii-3mae0mxi6K\|CID 73755245 | Inhibitor |
| PDXK | Phosphomethylphosphonic acid adenylate ester | Inhibitor |
| GSK3A | Kenpaullone\|6-Bromoindirubin-3′-oxime\|3-(2,4-Dichlorophenyl)-4-(1-methyl-1H-indol-3-yl)-1H- | Inhibitor\|.\| |
| | pyrrole-2,5-dione\|3-amino-6-[4-(2-methoxyethylsulfamoyl)phenyl]-N-pyridin-3-ylpyrazine-2- | Modulator |
| | carboxamide\|(2S,4S)-1-[(2R)-3-benzoylsulfinyl-2-methylpropanoyl]-4-phenylsulfanylpyrrolidine- | |
| | 2-carboxylic acid;(3S,4R)-3-hydroxy-2,2-dimethyl-4-(2-oxopyrrolidin-1-yl)-3,4-dihydrochromene- | |
| | 6-carbonitrile\|Lithium chloride\|Ferulic acid\|4-Benzyl-2-methyl-1,2,4-thiadiazolidine-3,5-dione\|6- | |
| | ((2-((4-(2,4-Dichlorophenyl)-5-(4-methyl-1H-imidazol-2-yl)pyrimidin-2- | |
| | yl)amino)ethyl)amino)nicotinonitrile\|N-(4-Methoxybenzyl)-N′-(5-nitro-1,3-thiazol-2-YL)urea\|3-(9- | |
| | Fluoro-2-(piperidine-1-carbonyl)-1,2,3,4-tetrahydro-[1,4]diazepino[6,7,1-hi]indol-7-yl)-4- | |
| | (imidazo[1,2-a]pyridin-3-yl)-1H-pyrrole-2,5-dione\|Lithium\|Valproic acid\|1-Benzyl-3-naphthalen-1- | |
| | ylurea\|5-[2,3-bis(fluoranyl)phenyl]-~{N}-[1-(2-methoxyethyl)piperidin-4-yl]methyl]-1~{ }- | |
| | indazole-3-carboxamide\|3-(5-Hydroxy-5,6,7,8-tetrahydroquinolin-2-yl)-2-oxoindoline-5- | |
| | carbonitrile\|2-Benzyl-4-(oxolan-2-ylmethyl)-1,2,4-thiadiazolidine-3,5-dione\|2-(2-chlorophenyl)- | |
| | 5,6-dimethyl-N-(5-methyl-1H-pyrazol-3-yl)pyrimidin-4-amine\|Indantadol hydrochloride\|1-(Pyrazin- | |
| | 2-yl)-3-(quinolin-4-yl)urea\|6-benzyl-4-N-(1H-indazol-6-yl)-2-N-(5-methyl-1H-pyrazol-3-yl)-1,3,5- | |
| | triazine-2,4-diamine\|1-(3,4-Dimethoxyphenyl)-2-(4-diphenylmethylpiperazinyl)ethanol | |
| | dihydrochloride\|2-Benzyl-4-(2-hydroxy-ethyl)-[1,2,4]thiadiazolidine-3,5-dione\|(4R)-4-(2- | |
| | methoxyphenyl)-3,7,7-trimethyl-4,6,8,9-tetrahydro-2H-pyrazolo[3,4-b]quinolin-5- | |
| | one\|Ceralifimod\|4-{[(1-{[2-(1-benzyl-3-methyl-1H-imidazol-3-ium-4-yl)-1-[(1-methylpiperidin-1- | |
| | ium-4-yl)carbamoyl]ethyl]carbamoyl}-5-({[12-({4-[6-(diethylamino)-3-(diethyliminiumyl)-3H- | |
| | xanthen-9-yl]-3-sulfobenzene}sulfonamido)dodecyl]carbamoyl}amino)pentyl)carbamoyl]methyl}- | |
| | 2-imino-3-methyl-2,3-dihydro-1,3-thiazol-3-ium tritrifluoroacetate\|5-(4-Methoxy-phenyl)-3- | |
| | (pyrimidin-2-ylamino)-1,5-dihydro-pyrazolo[4,3-c]pyridazin-6-one\|Sodium N- | |
| | methylphenylaminomethanesulfonate\|N-Methyl-N-(1-(4-phenoxybutyl)-3-pyrrolidinyl)-2- | |
| | benzothiazolamine\|1-(6-(3,6-Dihydro-2H-pyran-4-yl)pyridin-2-yl)-3-(8-fluoroquinolin-4-yl)urea\|N- | |
| | [2-[4,7-dichloro-6-(3-chlorothiophen-2-yl)pyrrolo[3,2-d]pyrimidin-5-yl]ethyl]-2,2,2- | |
| | trifluoroacetamide\|5-((6-Methylpyridin-2-yl)ethynyl)nicotinonitrile\|1-Pyrazin-2-yl-3-(6- | |
| | trifluoromethyl-quinolin-4-yl)-urea\|N-(3,4-dichlorophenyl)-1-(2-methoxyethyl)indazole-5- | |
| | carboxamide\|8-[(1-Acetylpiperidin-4-yl)amino]-6-amino-7,9-difluoro-1,1-dimethyl-5-oxo-2,3- | |
| | dihydropyrrolo[1,2-a]quinoline-4-carboxamide\|6-Amino-7-fluoro-4-oxo-8-[2-(pyridin-2- | |
| | ylamino)ethylamino]spiro[10-oxa-1-azatricyclo[7.4.1.05,14]tetradeca-2,5,7,9(14)-tetraene-13,1′- | |
| | cyclobutane]-3-carboxylic acid\|(+)-cis-H2-PAT\|GSK-3 Inhibitor X\|4-(7-butyl-5H-pyrrolo[2,3- | |
| | b]pyrazin-6-yl)phenol\|6-N-[2-[4-(2,4-dichlorophenyl)-5-imidazol-1-ylpyrimidin-2-yl]amino]ethyl]-3- | |
| | nitropyridine-2,6-diamine\|2-[(6s)-4-(4-Chlorophenyl)-2,3,9-Trimethyl-6h-[1,2]oxazolo[5,4- | |
| | C]thieno[2,3-E]azepin-6-Yl]acetamide\|(5-methoxy-1H-indol-3-yl)methanamine\|4-[4- | |
| | (Benzyloxy)piperidino]butyl-4-chlorobenzoate\|4-(Spiro[chromene-2,4″-piperidine]-4- | |
| | yl)phenol\|Tupichinol C\|(+)-(1R,1′S)-Berbamunine HCl\|leucettamine B | |
| DCLK3 | 1-[5-chloro-2-(trifluoromethyl)phenyl]methyl]-7-(2-piperazin-1-ylpyridin-4-yl)-3,4-dihydro-2H- | Inhibitor |
| | pyrido[2,3-b]pyrazine | |
| IKKB | Staurosporine\|4-Amino-[2,3′-bithiophene]-5-carboxamide\|5-Bromo-6-methoxy-beta-carboline\|5- | Inhibitor\| |
| | Amino-2-(4-methylphenyl)-1,3-oxazole-4-carboxamide\|5-Amino-2-phenyl-oxazole-4-carboxylic | Modulator |
| | acid amide\|2-Amino-5-phenylbenzamide\|3-Amino-1-benzothiophene-2-carboxamide\|2- | |
| | Aminoquinoline-3-carboxamide\|2-Amino-5-phenylthiophene-3-carboxamide\|6-Phenyl-thieno[3,2- | |
| | d]pyrimidin-4-ylamine\|3-Amino-5-(4-chlorophenyl)thiophene-2-carboxamide\|3,4-Dihydro-6-[4- | |
| | [[3-(methylsulfonyl)phenyl]methyl]amino]-5-(trifluoromethyl)-2-pyrimidinyl]amino]-2(1H)- | |
| | quinolinone\|(S)-N-(6-chloro-9H-pyrido[3,4-b]indol-8-yl)-4-(2-((2S,6R)-2,6-dimethylmorpholino)-2- | |
| | oxoethyl)-6,6-dimethylmorpholine-3-carboxamide mesylate\|N-(3,5-bis(trifluoromethyl)phenyl)-5- | |
| | chloro-2-hydroxybenzamide\|Kifunensine\|Fdt5cjg2AD\|Arsenic trioxide\|Parthenolide | |
| KGP1 | Rp-8-CPT-cGMPS | Inhibitor |
| TYK2 | Pyridone 6\|(6-Bromopyrazolo[1,5-a]pyrimidin-2-yl)-(2-methyl-4,5,7,8-tetrahydro-[1,3]thiazolo[4,5- | Inhibitor |
| | d]azepin-6-yl)methanone\|Brepocitinib\|Tyk2-IN-8\|Deucravacitinib\|N-(6-phenyl-1H-indazol-3- | |
| | yl)butyramide\|Cdk7-IN-2\|3-(2-(Pyridin-4-yl)ethyl)-1H-indole\|5,8-dichloro-7-[1-(2- | |
| | hydroxyacetyl)piperidin-4-yl]-methoxymethyl]-2-[(4-methoxy-6-methyl-2-oxo-1H-pyridin-3- | |
| | yl)methyl]-3,4-dihydroisoquinolin-1-one\|H-Gly-Gly-Thr-Thr-Gly-Gly-Thr-Gly-Thr-Gly-Gly-Thr-Thr- | |
| | Gly-Gly-OH\|Balapiravir\|(5-(3-methoxyphenyl)-1-pentyl-1H-pyrrol-3-yl)(naphthalen-1- | |
| | yl)methanone\|(3-(4-(6-Oxa-2-azaspiro[3.4]octan-2-ylmethyl)-3-methylphenoxy)azetidin-1-yl)(5- | |
| | phenyl-1,3,4-oxadiazol-2- | |
| | yl)methanone\|Enadoline\|Bifeprunox\|Fluspirilene\|[(4R,4aR,7S,7aR,12bS)-9-acetyloxy-3-methyl- | |
| | 1,2,3,4,4a,7,7a,13-octahydro-4,12-methanobenzofuro[3,2-e]isoquinolin-3-ium-7-yl]sulfate\|1- | |
| | Cyclohexyl-4-[3-(5-methoxy-1,2,3,4-tetrahydronaphthalen-1-yl)propyl]piperazine\|8-Bromo-3- | |
| | methyl-5-phenyl-2,3,4,5-tetrahydro-1H-benzo[d]azepin-7-ol\|Cyclofenil\|5-Butyl-8-phenyl-3H- | |
| | [1,2,4]triazolo[5,1-i]purine\|7 alpha,27-Dihydroxycholesterol\|1-(4-Iodophenyl)-2-pyrrolidin-1-yl- | |
| | pentan-1-one\|13,16,19,22-Tetraoxa-3,6-diazatricyclo[21.3.1.18,12]octacosa- | |
| | 1(27),2,6,8(28),9,11,23,25-octaene-27,28-diol\|Indiplon\|1-(3-carbamimidoylphenyl)-3-methyl-N- | |
| | [4-(2-sulfamoylphenyl)phenyl]-1H-pyrazole-5-carboxamide\|Rhein\|7,3′,4′-Trihydroxyflavone\|2-[4- | |
| | (3-Methyl-2-thienyl)phenyl]propionic acid\|Moxidectin\|Tiliroside\|(1S,14R,15R)-25- | |
| | (cyclopropylmethyl)-4,25-diazahexacyclo[13.7.3.01,14.03,12.05,10.017,22]pentacosa- | |
| | 3,5,7,9,11,17(22),18,20-octaen-20-ol\|2-[4-[2-(diethylamino)ethoxy]phenyl]-N-[(2,4- | |
| | difluorophenyl)methyl]-5-fluoropyrimidin-4-amine\|Nicotinamide riboside\|Gliclazide\|4-(4- | |
| | Iodobenzylidene)-1-(4-iodophenyl)pyrazolidine-3,5-dione\|5- | |
| | (Nonyloxy)tryptamine\|Plomestane\|Pyrotinib\|WtInsagyllgphavgnhpsfsdknglts-conh2 | |
| ULK1 | N-[(3R)-8-[5-chloro-4-[2-fluoro-6-(methylcarbamoyl)anilino]pyrimidin-2-yl]amino]-5,5-dimethyl-2- | Inhibitor |
| | oxo-3,4-dihydro-1H-1-benzazepin-3-yl]pyrrolidine-1-carboxamide | |
| MK12 | 2-(2-chloro-4-iodophenylamino)-N-cyclopropylmethoxy-3,4- | Inhibitor |
| | difluorobenzamide\|Phosphoaminophosphonic acid-adenylate | |
| | ester\|Phosphonothreonine\|Bisindolylmaleimide i\|2-(4-Fluorophenyl)-4-methoxy-3-(pyridin-4-YL)- | |
| | 1H-pyrrolo[2,3-B]pyridin-6-amine\|2-(Methylthio)-4-(4-fluorophenyl)-5-(4-pyridinyl)-1H- | |
| | imidazole\|4-[5-(4-Fluorophenyl)-2-(4-methanesulfinyl-benzylsulfanyl)-3H-imidazol-4- | |
| | yl]pyridine\|p38 MAP Kinase Inhibitor III\|Bisindolylmaleimide IV\|(S)-4-(2-(N-methylisoquinoline-5- | |
| | sulfonamido)-3-oxo-3-(4-phenylpiperazin-1-yl)propyl)phenyl isoquinoline-5-sulfonate\|4,5,6,7- | |
| | Tetrabromobenzotriazole\|bisindolylmaleimide\|X\|Indiplon\|KT5720\|4-(5-(4-Fluorophenyl)-3-(4- | |
| | piperidyl)imidazol-4-yl)pyridine\|Pramiconazole\|p38alpha Inhibitor 1\|Neflamapimod\|#N/A | |
| STK36 | [4-Amino-2-[4-(4-methyl-1-piperazinyl)phenyl]amino]-5-thiazolyl](2,3-dihydro-1,4-benzodioxin-6- | Inhibitor |
| | yl)methanone | |
| PK3CA | 5-(7-(methylsulfonyl)-2-morpholino-6,7-dihydro-5H-pyrrolo[2,3-d]pyrimidin-4-yl)pyrimidin-2- | Inhibitor\| |
| | amine\|5-(2-((4-(Methylsulfonyl)piperazin-1-yl)methyl)-8-morpholinoimidazo[1,2-a]pyrazin-6- | Modulator |
| | yl)pyrimidin-2-amine\|[Sar1Gly]GAL-B2\|N-(4-(4-(2-(difluoromethyl)-4-methoxy-1H- | |
| | benzo[d]imidazol-1-yl)-6-morpholino-1,3,5-triazin-2-yl)phenyl)-2- | |
| | (dimethylamino)ethanesulfonamide\|Unii- | |
| | rca38L4HB5\|Serabelisib\|Samotolisib\|Taselisib\|Buparlisib\|Alpelisib\|Copanlisib | |
| AVR2B | 2-Cyclohexyl-N-(1-isopropylpiperidin-4-yl)-6-methoxy-7-(3-(pyrrolidin-1-yl)propoxy)quinazolin-4- | Antagonist |
| | amine | |
| CSK22 | [4,5,6,7-Tetrabromo-2-(Dimethylamino)-1h-Benzimidazol-1-Yl]acetic Acid\|4-Amino-N-(trans-4- | Inhibitor |
| | hydroxycyclohexyl)-7-(2-fluoro-4-pyridinyl)pyrrolo[2,1-f][1,2,4]triazine-5-carboxamide\|ethyl N-[4- | |
| | [(4-amino-7-pyridin-4-ylpyrrolo[2,1-f][1,2,4]triazine-5-carbonyl)amino]cyclohexyl]carbamate\|2-[(3- | |
| | Cyano-1-methylindazol-5-yl)amino]-4-(cyclopropylamino)imidazo[2,1-f][1,2,4]triazine-7- | |
| | carbonitrile\|ethyl N-[4-[4-amino-7-(2-methoxypyridin-4-yl)pyrrolo[2,1-f][1,2,4]triazine-5- | |
| | carbonyl]amino]cyclohexyl]carbamate\|2-[2-Chloro-5-cyano-3-(3-hydroxypyrrolidin-1-yl)anilino]-4- | |
| | (cyclopropylamino)imidazo[2,1-f][1,2,4]triazine-7-carbonitrile\|2-[2-Chloro-5-cyano-3-(6-methyl- | |
| | 2,6-diazaspiro[3.3]heptan-2-yl)anilino]-4-(cyclopropylamino)imidazo[2,1-f][1,2,4]triazine-7- | |
| | carbonitrile | |
| CDC7 | 2-(2-Aminopyrimidin-4-yl)-4-(2,4-dichlorophenyl)-1,3-thiazole-5-carboxamide\|2-(2- | Inhibitor\| |
| | Aminopyrimidin-4-yl)-4-(2-chloro-4-methoxyphenyl)-1,3-thiazole-5-carboxamide\|(7S)-2-(2- | Modulator |
| | aminopyrimidin-4-yl)-7-(2-fluoroethyl)-1,5,6,7-tetrahydro-4H-pyrrolo[3,2-c]pyridin-4-one\|5-(2- | |
| | aminopyrimidin-4-yl)-2-phenyl-1H-pyrrole-3-carboxamide\|2-Pyridin-4-yl-1,5,6,7- | |
| | tetrahydropyrrolo[3,2-c]pyridin-4-one\|Simurosertib\|8-Chloro-2-[(2s)-Pyrrolidin-2- | |
| | Yl][1]benzofuro[3,2-D]pyrimidin-4(3h)-One\|Albaconazole | |
| DAPK3 | 3-(1H-indol-5-yl)-5-(4-((4-methylpiperazin-1-yl)methyl)phenyl)-1H-pyrrolo[2,3-b]pyridine | Inhibitor |
| PRKDC | c[Glu26-Lys30][Gly8]GLP-1(7-37)-NH2\|2-(4-ethylpiperazin-1-yl)-N-[4-(2-morpholin-4-yl-4- | .\|Inhibitor |
| | oxochromen-8-yl)dibenzothiophen-1-yl]acetamide\|1-(2-Hydroxy-4- | |
| | morpholinophenyl)ethanone\|DNA-PK Inhibitor VIN-[(E)-(6-Bromoimidazo[1,2-a]pyridin-3- | |
| | yl)methylideneamino]-N,2-dimethyl-5-nitrobenzenesulfonamide\|1-cyclopentyl-3-(1H-pyrrolo[2,3- | |
| | b]pyridin-5-yl)-1H-pyrazolo[3,4-d]pyrimidin-4-amine\|2-Morpholin-4-yl-8-thiophen-2-ylchromen-4- | |
| | one\|8-(4-Dibenzothienyl)-2-(4-morpholinyl)-4H-1-benzopyran-4-one\|2-Morpholin-4-yl-8-thiophen- | |
| | 3-ylchromen-4-one\|7-Hydroxy-2-(morpholin-4-yl)chromen-4-one\|2-morpholinobenzo[h]quinolin- | |
| | 4(1H)-one\|2-morpholino-6-phenyl-4H-thiopyran-4-one\|2-morpholino-6-phenyl-4H-pyran-4-one\|8- | |
| | (4-Acetylphenyl)-2-morpholin-4-ylchromen-4-one\|2-Morpholin-4-yl-8-phenoxathiin-4-ylchromen- | |
| | 4-one\|8-(1H-indol-5-yl)-2-morpholin-4-ylchromen-4-one\|8-(4-Hydroxyphenyl)-2-morpholin-4- | |
| | ylchromen-4-one\|8-(3-Hydroxyphenyl)-2-morpholin-4-ylchromen-4-one\|8-(4-Chlorophenyl)-2- | |
| | morpholin-4-ylchromen-4-one\|8-Dibenzofuran-4-yl-2-morpholin-4-ylchromen-4-one\|8-Furan-2-yl- | |
| | 2-morpholin-4-ylchromen-4-one\|2-Morpholin-4-yl-8-naphthalen-2-ylchromen-4-one\|8-Biphenyl-2- | |
| | yl-2-morpholin-4-ylchromen-4-one\|8-(2-Acetylphenyl)-2-morpholin-4-ylchromen-4-one\|2- | |
| | Morpholin-4-yl-8-styrylchromen-4-one\|7-Phenyloxyethoxy-2-morpholin-4-yl-chromen-4-one\|7- | |
| | Ethyloxyethoxy-2-morpholin-4-yl-chromen-4-one\|7-Phenylethoxy-2-morpholin-4-yl-chromen-4- | |
| | one\|7-Benzoyloxy-2-(morpholin-4-yl)-chromen-4-one\|7-Benzyloxy-2-(morpholin-4-yl)-chromen-4- | |
| | one\|7-Propoxy-2-(morpholin-4-yl)-chromen-4-one\|6-(4-Hydroxyphenyl)-2-morpholin-4- | |
| | ylchromen-4-one\|6-(3-Hydroxyphenyl)-2-morpholin-4-ylchromen-4-one\|2-Morpholin-4-yl-6- | |
| | naphthalen-1-ylchromen-4-one\|2-Morpholin-4-yl-6-thianthren-1-ylchromen-4-one\|6-[(E)-2-(4- | |
| | fluorophenyl)ethenyl]-2-morpholin-4-ylchromen-4-one\|2-Morpholin-4-yl-6-(2- | |
| | phenylphenyl)chromen-4-one\|6-(2-Acetylphenyl)-2-morpholin-4-ylchromen-4-one\|2-(2-Fluoro- | |
| | phenyl)-benzo[h]chromen-4-one\|2-(3-Fluoro-phenyl)-benzo[h]chromen-4-one\|2-(4-Amino- | |
| | phenyl)-benzo[h]chromen-4-one\|2-Pyridin-3-yl-benzo[h]chromen-4-one\|2-Morpholin-4- | |
| | ylpyrido[1,2-a]pyrimidin-4-one\|2-Thiomorpholin-4-yl-pyrido[1,2-a]pyrimidin-4-one\|2-Morpholin-4- | |
| | yl-benzo[h]chromene-4-thione\|2-thiomorpholino-4H-benzo[h]chromen-4-one\|2-morpholino-4H- | |
| | benzo[g]chromen-4-one\|3-morpholino-1H-benzo[f]chromen-1-one\|4-Morpholin-4-yl- | |
| | benzo[g]chromen-2-one\|4-Thiomorpholin-4-yl-benzo[g]chromen-2-one\|4-Morpholin-4-yl- | |
| | benzo[h]chromen-2-one\|1-Morpholin-4-yl-benzo[f]chromen-3-one\|2-morpholino-4H-pyrimido[2,1- | |
| | alisoquinolin-4-one\|8-Methyl-2-morpholin-4-yl-chromen-4-one\|7-Methoxy-2-morpholin-4-yl- | |
| | chromen-4-one\|8-Methoxy-2-morpholin-4-yl-chromen-4-one\|2-Morpholin-4-yl-6-phenyl-chromen- | |
| | 4-one\|8-Phenyl-2-piperidin-1-yl-chromen-4-one\|8-Phenyl-2-thiomorpholin-4-yl-chromen-4- | |
| | one\|4H-Naphtho(1,2-b)pyran-4-one,2-(1-piperidinyl)-\|2-(4-Morpholinyl)-4H-1-benzopyran-4- | |
| | one\|2-(Morpholin-4-yl)-benzo[h]chromen-4-one\|4H-Naphtho(1,2-b)pyran-4-one,2-(1- | |
| | pyrrolidinyl)-\|2-[1,4]Oxazepan-4-yl-benzo[h]chromen-4-one\|2-(2-Methyl-morpholin-4-yl)- | |
| | benzo[h]chromen-4-one\|4-Morpholin-4-yl-chromen-2-one\|4-Piperidin-1-yl-chromen-2-one\|6- | |
| | Methyl-4-morpholin-4-yl-chromen-2-one\|8-Methyl-4-morpholin-4-yl-chromen-2-one\|6-methoxy-4- | |
| | morpholino-2H-chromen-2-one\|7-Methoxy-4-morpholin-4-yl-chromen-2-one\|8-Methoxy-4- | |
| | morpholin-4-yl-chromen-2-one\|6-Chloro-4-morpholin-4-yl-chromen-2-one\|alpha- | |
| | Naphthoflavone\|6-(4-Chlorophenyl)-2-morpholin-4-ylpyridin-4-ol\|6-tert-Butyl-2-morpholin-4-yl-4H- | |
| | thiopyran-4-one\|6-isopropyl-2-morpholin-4-yl-4H-thiopyran-4-one\|6-(4-chlorophenyl)-4- | |
| | morpholino-2H-pyran-2-one\|6-(4-methoxyphenyl)-4-morpholino-2H-pyran-2-one\|4-morpholino-6- | |
| | phenyl-2H-pyran-2-one\|2-(4-methoxyphenyl)-6-morpholino-4H-pyran-4-one\|2-(3- | |
| | methoxyphenyl)-6-morpholino-4H-pyran-4-one\|2-(2-methoxyphenyl)-6-morpholino-4H-pyran-4- | |
| | one\|2-(4-fluorophenyl)-6-morpholino-4H-pyran-4-one\|2-(4-chlorophenyl)-6-morpholino-4H-pyran- | |
| | 4-one\|2-morpholino-6-styryl-4H-pyran-4-one\|2-(3-acetylphenyl)-6-morpholino-4H-pyran-4-one\|4- | |
| | (6-morpholino-4-oxo-4H-pyran-2-yl)benzonitrile\|3-(6-morpholino-4-oxo-4H-pyran-2- | |
| | yl)benzonitrile\|2-(2-aminophenyl)-6-morpholino-4H-pyran-4-one\|2-Biphenyl-4-yl-6-morpholin-4- | |
| | yl-pyran-4-one\|3-(6-morpholino-4-oxo-4H-pyran-2-yl)benzoic acid\|3-(6-morpholino-4-oxo-4H- | |
| | pyran-2-yl)benzaldehyde\|2-morpholino-6-(naphthalen-2-yl)-4H-pyran-4-one\|2-morpholino-6- | |
| | (naphthalen-1-yl)-4H-pyran-4-one\|2-(benzofuran-2-yl)-6-morpholino-4H-pyran-4-one\|2-(1H-indol- | |
| | 5-yl)-6-morpholino-4H-pyran-4-one\|2-(furan-2-yl)-6-morpholino-4H-pyran-4-one\|2-morpholino-6- | |
| | (thiophen-3-yl)-4H-pyran-4-one\|2-morpholino-6-(thiophen-2-yl)-4H-pyran-4-one\|2-Morpholino-6- | |
| | (thianthren-1-yl)-4H-pyran-4-one\|2-Dibenzofuran-4-yl-6-morpholin-4-yl-pyran-4-one\|2- | |
| | morpholino-6-(quinolin-8-yl)-4H-pyran-4-one\|2-Methylbenzo[d]thiazole-5-carboxylic | |
| | acid\|Nedisertib\|7-Methyl-2-[(7-methyl-[1,2,4]triazolo[1,5-a]pyridin-6-yl)amino]-9-(oxan-4-yl)purin- | |
| | 8-one | |
| SIK3 | 1-(2,4-Dimethoxyphenyl)-3-(2,6-dimethylphenyl)-1-[6-[4-(4-methylpiperazin-1- | Inhibitor |
| | yl)anilino]pyrimidin-4-yl]urea | |
| KKCC2 | 7-Oxo-7h-Benzimidazo[2,1-A]benz[de]isoquinoline-3-Carboxylic Acid\|Spiro[piperidine-4,3′- | Inhibitor |
| | [3H]pyrido[3,4-b]indole]-4′-carbonitrile,7′,8′-dichloro-1′,2′,4′,9′-tetrahydro-9′-methyl-1′-oxo-\|4-(4- | |
| | (cyclopentyloxy)-5-(4-(methylcarbamoyl)phenyl)-7H-pyrrolo[2,3-d]pyrimidin-2-ylamino)-3- | |
| | methoxy-N-methylbenzamide\|4-((4-(Cyclopentyloxy)-5-(2-methylbenzo[d]oxazol-6-yl)-7H- | |
| | pyrrolo-[2,3-d]pyrimidin-2-yl)amino)-3-methoxy-N-methylbenzamide\|4-(4-(cyclopentyloxy)-5-(2- | |
| | methylbenzo[d]oxazol-6-yl)-7H-pyrrolo[2,3-d]pyrimidin-2-ylamino)-N-(2-hydroxyethyl)-3- | |
| | methoxybenzamide | |
| UFO | Furnidipine\|N-[4-(6,7-dimethoxyquinolin-4-yl)oxy-3-fluorophenyl]-4-ethoxy-1-(4-fluoro-2- | .\|Inhibitor\| |
| | methylphenyl)pyrazole-3-carboxamide\|Ceftobiprole medocaril free acid\|Belvarafenib\|N,N,N′,N′- | Modulator |
| | Tetramethyl-p-phenylenediamine dihydrochloride\|8-(3-Hydroxyphenyl)-2-morpholin-4- | |
| | ylchromen-4-one\|cyclo[Cys6,Cys10]N/OFQ(1-13)NH2\|7-fluoro-2-(4-fluorophenyl)-4H-chromene- | |
| | 4-thione\|N-[4-(6,7-dimethoxyquinolin-4-yl)oxy-3-fluorophenyl]-3-(4-fluorophenyl)-2,4-dioxo-1- | |
| | propan-2-ylpyrimidine-5-carboxamide\|(1s,2s)-2-(3,4-Dichlorobenzoyl)cyclopropanecarboxylic | |
| | Acid\|(2S)-2-[[(2S)-2-[(2S)-2-[[(2S,3R)-2-[(2S)-2-[(2S)-2-[(2S)-2-[(2S)-2-[(2S)-2-[(2- | |
| | acetamidoacetyl)amino]propanoyl]amino]-5-amino-5-oxopentanoyl]amino]-3- | |
| | phenylpropanoyl]amino]-3-hydroxypropanoyl]amino]-6-aminohexanoyl]amino]-3- | |
| | hydroxybutanoyl]amino]propanoyl]amino]propanoyl]amino]-6-aminohexanoic acid\|N-(3-(9H- | |
| | carbazol-9-yl)-2-hydroxypropyl)-N-(furan-2-ylmethyl)methanesulfonamide\|2-((5-chloro-2-((4-((4- | |
| | methylpiperazin-1-yl)methyl)phenyl)amino)pyrimidin-4-yl)amino)-N,N- | |
| | dimethylbenzenesulfonamide\|N~1~-({5-[4,4-bis(ethoxymethyl)cyclohexyl]-1H-pyrazol-4- | |
| | yl}methyl)-N~1~,N~2~-dimethylethane-1,2-diamine\|N-(5-((6,7-Dimethoxyquinolin-4- | |
| | yl)oxy)pyridin-2-yl)-2,5-dioxo-1-phenyl-1,2,5,6,7,8-hexahydroquinoline-3- | |
| | carboxamide\|Bemcentinib\|N-(3-fluoro-4-(2-(1-methyl-1H-imidazol-4-yl)thieno[3,2-b]pyridin-7- | |
| | yloxy)phenylcarbamothioyl)-2-phenylacetamide\|1-(Benzhydrylamino)ethaniminium | |
| | bromide\|Gilteritinib | |
| DYR1B | Methyl 9-(4-bromo-2-fluoroanilino)-[1,3]thiazolo[5,4-f]quinazoline-2-carboximidate\|Methyl 9-(4- | Inhibitor |
| | chloroanilino)-[1,3]thiazolo[5,4-f]quinazoline-2-carboximidate\|4-(1,3-benzodioxol-5-yl)-N-[(3,5- | |
| | dichlorophenyl)methyl]pyrimidin-2-amine\|Dcdpc\|Nabumetone\|3-(2,7-Dimethoxyacridin-9- | |
| | ylthio)propan-1-amine\|#N/A\|2,3-Diphenyl-1H-indole\|2-[(4-{2-[(4- | |
| | Cyclohexylbutyl)(cyclohexylcarbamoyl)amino]ethyl}phenyl)sulfanyl]-2-methylpropanoic | |
| | acid\|Bicyclo(2.2.2)octane-1-carboxylic acid,4-((((3′R,4′S,5′R)-6″-chloro-4′-(3-chloro-2- | |
| | fluorophenyl)-1′-ethyl-1″,2″-dihydro-2″-oxodispiro(cyclohexane-1,2′-pyrrolidine-3′,3″-(3H)indol)-5′- | |
| | yl)carbonyl)amino)-\|Methyl 9-((2-fluoro-4-methoxyphenyl)amino)thiazolo[5,4-f]quinazoline-2- | |
| | carbimidate\|Methyl 9-(2,4-dichloroanilino)-[1,3]thiazolo[5,4-f]quinazoline-2-carboximidate\|9- | |
| | methoxy-11H-pyrido[4,3-a]carbazole-6-carbonitrile\|1,3,4(2H)-Isoquinolinetrione,2-(2-propenyl)- | |
| | \|3-amino-N-(5-phenyl-1,3,4-thiadiazol-2-yl)-6,7,8,9-tetrahydro-5H-cyclohepta[b]thieno[3,2- | |
| | e]pyridine-2-carboxamide\|1,9-dimethoxy-11H-pyrido[4,3-a]carbazole-6-carbonitrile\|Methyl 9-(4- | |
| | methylanilino)-[1,3]thiazolo[5,4-f]quinazoline-2-carboximidate\|N-butyl-2-(1H-indol-3-yl)-2- | |
| | oxoacetamide\|Methyl 9-(4-chloro-2-fluoroanilino)-[1,3]thiazolo[5,4-f]quinazoline-2- | |
| | carboximidate\|Methyl 9-(2,4-difluoroanilino)-[1,3]thiazolo[5,4-f]quinazoline-2- | |
| | carboximidate\|Vyxeos | |
| KC1G2 | 2-[2-(4-fluoro-2-methylphenoxymethyl)-5-methoxypyridin-4-yl]-1H,4H,5H,6H,7H-pyrrolo[3,2- | Inhibitor |
| | c]pyridin-4-one | |
| ABL2 | N-[3-(Trifluoromethyl)-4-[(4-methylpiperazino)methyl]phenyl]-3-[(imidazo[1,2-a]pyrazine-5- | Inhibitor |
| | yl)ethynyl]-4-methylbenzamide | |
| KPCI | 3-(4-{[5-Fluoro-2-(4-methylphenyl)phenyl]methoxy}phenyl)propanoic acid[(1S,2S,3R,4S)-4-(5- | Inhibitor |
| | amino-4-carbamoylimidazol-1-yl)-2,3-dihydroxycyclopentyl]methyl dihydrogen phosphate | |
| KS6A1 | 2-((2-((1-(2-(Dimethylamino)acetyl)-5-methoxyindolin-6-yl)amino)-7H-pyrrolo[2,3-d]pyrimidin-4- | Inhibitor |
| | yl)amino)-6-fluoro-N-methylbenzamide\|2-[3,4-Dioxo-2-(pyridin-4-ylamino)cyclobuten-1- | |
| | yl]amino]-2-(3-hydroxyphenyl)acetamide\|2-((3,5-difluoro-4-hydroxyphenyl)amino)-8-isopentyl- | |
| | 5,7-dimethyl-7,8-dihydropteridin-6(5H)-one\|7beta-[2-(2-Aminothiazol-4-yl)-2-(z)- | |
| | (hydroxyimino)acetamido]-3-[(pyrazol-4-yl)methylthio]-3-cephem-4-carboxylic | |
| | acid\|Ethambutol\|FIt-3 Inhibitor III\|Ac-YR[CEH(pCI-dF)RWC]-NH2\|4-(6,11-dioxo-3H-naphtho[2,3- | |
| | e]benzimidazol-2-yl)benzonitrile | |
| NTRK2 | 5-oxo-1-tosyl-2,5-dihydro-1Hpyrrol-2-yl acetate\|(Z)-1-(3-((3-((1H-pyrrol-2-yl)methylene)-2- | Modulator\| |
| | oxoindolin-6-yl)amino)-4-methylphenyl)-3-(2-fluoro-5-(trifluoromethyl)phenyl)urea\|(2R)-2-[5-[6- | Inhibitor |
| | amino-5-[(1R)-1-[5-fluoro-2-(triazol-2-yl)phenyl]ethoxy]pyridin-3-yl]-4-methyl-1,3-thiazol-2- | |
| | yl]propane-1,2-diol\|5-chloro-N2-(1-(5-fluoropyridin-2-yl)ethyl)-N4-(5-isopropoxy-1H-pyrazol-3- | |
| | yl)pyrimidine-2,4-diamine\|(3S,4S,5S)-5-(Hydroxymethyl)piperidine-3,4-diol\|5-[4-[2-(4-oxo-2H- | |
| | 1,3-benzoxazin-3-yl)ethoxy]phenyl]methyl]-1,3-thiazolidine-2,4- | |
| | dione\|Aekkdegpyrmehfrwgsppkd\|20-Amino-14-fluoro-4,17-dimethyl-7,10,18-trioxa-4,5,21- | |
| | triazatetracyclo[17.3.1.02,6.011,16]tricosa-1(23),2,5,11(16),12,14,19,21-octaene-3- | |
| | carbonitrile\|3-amino-6-methyl-N-pyridin-2-ylpyrazine-2-carboxamide\|2-[6-(4- | |
| | Chlorophenoxy)hexyl]-1-cyano-3-pyridin-4-ylguanidine\|MK-2461\|Entrectinib\|Larotrectinib\|N-(6- | |
| | fluoro-3-pyridyl)-1-[4-[(5-isopropyl-1H-pyrazol-3-yl)amino]-6,7-dihydro-5H- | |
| | cyclopenta[d]pyrimidin-2-yl]pyrrolidine-2-carboxamide\|4-[3-amino-4-(oxan-4-yloxy)-1H-indazol-6- | |
| | yl]-2-(4-cyclohexylpiperazin-1-yl)-N-methylbenzamide\|4-[3-amino-4-(1,1,1-trifluoropropan-2- | |
| | yloxy)-1H-indazol-6-yl]-2-(4-phenylpiperazin-1-yl)benzamide\|4-[3-amino-4-(oxan-4-yloxy)-1H- | |
| | indazol-6-yl]-2-(4-phenylpiperazin-1-yl)benzamide\|XX8E5WP7FS | |
| INSR | 4-Hexynoic acid,2-(((4-((4-methoxybenzoyl)amino)phenyl)sulfonyl)amino)-6-(4-morpholinyl)- | Modulator\|.\| |
| | IpGlu-Gln-Arg-Leu-Gly-Asn-Gln-Trp-Ala-Val\|(6-Methoxy-9-tricyclo[9.4.0.03,8]pentadeca- | Inhibitor\| |
| | 1(15),3(8),4,6,11,13-hexaenyl)methylurea\|3-(6-Amino-purin-9-yl)-6-o-tolyl-hexan-2-ol\|5-Me- | Agonist\| |
| | IAA\|cacospongionolide E\|Cefsulodine\|2″,4″-Dicyanobiphenyl-4-yl sulfamate\|2-((2-((1-(2- | Activator\| |
| | (Dimethylamino)acetyl)-5-methoxyindolin-6-yl)amino)-7H-pyrrolo[2,3-d]pyrimidin-4-yl)amino)-6- | Enhancer\| |
| | fluoro-N-methylbenzamide\|(1S,2S,3R,4R)-3-[5-Chloro-2-(1-ethyl-6-methoxy-2-oxo-2,3,4,5- | Stimulator\| |
| | tetrahydro-1H-benzo[b]azepin-7-ylamino)-pyrimidin-4-ylamino]-bicyclo[2.2.1]hept-5-ene-2- | Binder |
| | carboxylic acid amide\|Phosphomethylphosphonic acid adenylate ester\|4-((1H-indazol-6- | |
| | ylamino)methyl)benzene-1,2-diol\|4-((Naphthalen-2-ylamino)methyl)benzene-1,2-diol\|5-chloro- | |
| | N4-(2-(isopropylsulfonyl)phenyl)-N2-(2-methoxy-4-(4-(4-methylpiperazin-1-yl)piperidin-1- | |
| | yl)phenyl)pyrimidine-2,4-diamine\|ethyl 6-ethyl-2-methyl-4-oxo-1H-quinoline-3- | |
| | carboxylate\|Daedalin A\|2-Thiophenecarboxamide,N-(5-((4-(2-hydroxyacetyl)-1- | |
| | piperazinyl)methyl)-2-((1E)-2-(1H-indazol-3-yl)ethenyl)phenyl)-3-methyl-,4- | |
| | methylbenzenesulfonate (1:1)\|(5Z,8Z,11Z,14Z)-16-hydroxyicosa-5,8,11,14-tetraenoic acid\|Acetic | |
| | Acid\|4-(4-(4-Tert-butylphenylamino)phthalazin-1-yl)benzamide\|Methyl 2-cyclopropyl-2-[6- | |
| | (cyclopropylmethoxy)-5-(3,3-difluoroazetidin-1-yl)pyrazine-2- | |
| | carbonyl]amino]propanoate\|Lrhylnlltrqry-NH2\|1-Cyano-2-(2-Methyl-1-Benzofuran-5-Yl)-3-[(3s)-2- | |
| | Oxo-1-(2-Oxo-2-Pyrrolidin-1-Ylethyl)azepan-3-Yl]guanidine\|Alprenoxime | |
| | hydrochloride\|Guanosine-2′,3′-O-ethylidenephosphonate\|Nvp-aew541\|beta-D-glucosyl-N- | |
| | (tetracosanoyl)sphingosine\|Leurubicin\|(Z)-1-(oxazol-2-yl)octadec-9-en-1-one\|5-[(R)-1- | |
| | Aminoethyl]isoxazol-3-ol\|Tesofensine\|Sofosbuvir\|Atpo\|Mitoglitazone\|5-(4-(2-(3-Methoxyphenyl)- | |
| | 2-oxoethoxy)benzyl)thiazolidine-2,4-dione\|(1R,2R)-2-(1H-Imidazol-4-yl)-1-methyl- | |
| | propylamine\|Metformin glycinate\|#N/A\|Raclopride\|O\|Insulin glulisine\|Dalanated insulin\|9,10- | |
| | dihydroanthracene(DHA),2b\|Insulin lispro\|3-m-Tolyl-1,5-naphthyridine\|Triacyl lipid A OM-174 | |
| CDK4 | Roniciclib\|[4-Amino-2-[4-(4-methyl-1-piperazinyl)phenyl]amino]-5-thiazolyl](2,3-dihydro-1,4- | Inhibitor\| |
| | benzodioxin-6-yl)methanone\|2-fluoro-4-(4-(1-isopropyl-2-methyl-1H-imidazol-5-yl)pyrimidin-2- | Modulator |
| | ylamino)-N-methylbenzamide\|4-(6-Cyclohexylmethoxy-9H-purin-2-ylamino)-N,N- | |
| | diethylbenzamide\|Cdk4 Inhibitor\|1,4-dimethoxy-10H-acridine-9- | |
| | thione\|Ryuvidine\|Fascaplysine\|Pyrazolopyridazine 1\|Pyrazolopyridazine 2\|Diarylurea deriv. | |
| | 14b\|Diarylurea deriv. 14\|Diarylurea deriv. 14a\|Diarylurea deriv. 15\|2-(4-Methoxyphenyl)-3- | |
| | phenylmaleimide\|O6-Cyclohexylmethoxy-2-(4′-sulphamoylanilino) purine\|#N/A\|2-(1H-Indole-3- | |
| | yl)-3-phenylmaleimide\|1H-Pyrrole-2,5-dione,3,4-bis(4-methoxyphenyl)-\|3,4-diphenyl-1H-pyrrole- | |
| | 2,5-dione\|N-(2-(1H-Indol-3-yl)ethyl)biphenyl-4-carboxamide\|10-Hydroxy-18- | |
| | methoxybetaenone\|1-(1-Phenyl-2-(pyridin-2-yl)ethyl)piperazine\|8-Bicyclo[2.2.1]hept-2-yl-2-{4-[4- | |
| | (3-hydroxypropyl)piperidin-1-yl]phenylamino}-8H-pyrido[2,3-d]pyrimidin-7-one\|2-({2-[(R)-2- | |
| | Amino-3-(4-hydroxy-2,6-dimethyl-phenyl)-propionyl]-1,2,3,4-tetrahydro-isoquinoline-3-carbonyl}- | |
| | amino)-propionic acid\|(4-Amino-2-((1-(methylsulfonyl)piperidin-4-yl)amino)pyrimidin-5-yl)(2,3- | |
| | difluoro-6-methoxyphenyl)methanone\|ZK-Cdk\|Voruciclib\|Zidovudine\|Rgb-286638\|3- | |
| | Pyridinemethanamine,5-(3-(4,6-difluoro-1H-benzimidazol-2-yl)-1H-indazol-5-yl)-N-ethyl-4- | |
| | methyl-[N-(6,6-dimethyl-5-(1-methylpiperidine-4-carbonyl)-1,4,5,6-tetrahydropyrrolo[3,4- | |
| | c]pyrazol-3-yl)-3-methylbutanamide\|Maralixibat\|N-[4-[(4-methylpiperazin-1-yl)methyl]phenyl]-4- | |
| | (7H-pyrrolo[2,3-d]pyrimidin-4-ylamino)-1H-pyrazole-5-carboxamide\|Unii- | |
| | uch3qqh1C0\|Ceftobiprole medocaril free acid\|Riviciclib hydrochloride\|1H-pyrrole-2,5-dione,3-(1- | |
| | methyl-1h-indol-3-yl)-4-(1-methyl-6-nitro-1h-indol-3-yl)-\|2-(2-chlorophenyl)-5,7-dihydroxy-8- | |
| | [(2R,3S)-2-(hydroxymethyl)-1-methylpyrrolidin-3-yl]chromen-4- | |
| | one\|Lerociclib\|Ribociclib\|Palbociclib\|Trilaciclib\|Apremilast\|Abemaciclib\|Ribociclib succinate\|ethyl | |
| | (7S)-11-oxo-14-(2-trimethylsilylethynyl)-2,4,10-triazatetracyclo[10.4.0.02,6.07,10]hexadeca- | |
| | 1(16),3,5,12,14-pentaene-5-carboxylate\|4-((1R,2R)-2-Hydroxycyclohexyl)- | |
| | 2(trifluoromethyl)benzonitrile\|4-[3-Hydroxyanilino]-6,7-dimethoxyquinazoline\|N-(4- | |
| | (Diethylamino)benzyl)-4-methoxy-N-(p-tolyl)benzenesulfonamide\|(2,5-Dimethoxybenzyl)(2- | |
| | phenylethyl)amine\|N-(9-chloro-2-(furan-2-yl)-[1,2,4]triazolo[1,5-c]quinazolin-5-yl)-2- | |
| | phenylacetamide\|Cpzen-45\|7-(4-tert-butylphenyl)-2-[5-(3,8-diazabicyclo[3.2.1]octane-3- | |
| | carbonyl)pyridin-2-yl]amino]-N,N-dimethylpyrrolo[2,3-d]pyrimidine-6-carboxamide\|4-[5-(4- | |
| | dimethylamino-piperidin-1-yl)-3H-imidazo[4,5-b]pyridine-2-carbonyl]-2-isoquinolin-4-yl- | |
| | benzonitrile\|7-cyclopentyl-2-[5-(4-isopropyl-piperazin-1-yl)-pyridin-2-ylamino]-7H-pyrrolo[2,3- | |
| | d]pyrimidine-6-carboxylic acid dimethylamide\|4-[4-[2-[5-[(5-tert-butyl-1,3-oxazol-2- | |
| | yl)methylsulfanyl]-1,3-thiazol-2-yl]amino]-2-oxoethyl]phenoxy]-N-hydroxybutanamide\|4-[(5- | |
| | Piperazin-1-ylpyridin-2-yl)amino]spiro[1,3,5,11-tetrazatricyclo[7.4.0.02,7]trideca-2,4,6,8-tetraene- | |
| | 13,1′-cyclohexane]-10-one\|2-(5,7-dichloro-1H-indol-3-yl)ethanamine\|Fascaplysin | |
| SRC | 1-(tert-Butyl)-3-(2-((4-(diethylamino)butyl)amino)-6-(3,5-dimethoxyphenyl)pyrido[2,3-d]pyrimidin- | Inhibitor\| |
| | 7-yl)urea\|N-(4-Phenylamino-Quinazolin-6-Yl)-Acrylamide\|N-[4-[(4-ethylpiperazin-1-yl)methyl]-3- | Modulator |
| | (trifluoromethyl)phenyl]-4-methyl-3-(1H-pyrrolo[2,3-b]pyridin-4-yloxy)benzamide\|3,4- | |
| | Methylenedioxy-beta-nitrostyrene\|1-cyclopentyl-3-(1H-pyrrolo[2,3-b]pyridin-5-yl)-1H- | |
| | pyrazolo[3,4-d]pyrimidin-4-amine\|2,6-Dimethylphenyl(2,4-dimethoxyphenyl)(2-((4-(4- | |
| | methylpiperazin-1-yl)phenyl)amino)pyrimidin-4-yl)carbamate\|N-(5-methyl-1H-pyrazol-3-yl)-2- | |
| | phenylquinazolin-4-amine\|3-[(4-chlorophenyl)-(1H-imidazol-2-yl)methylidene]-5-[(1- | |
| | ethylpiperidin-4-yl)amino]-1H-indol-2-one\|N-(2-chloro-6-methylphenyl)-2-[(2,6-dimethylpyrimidin- | |
| | 4-yl)amino]-1,3-thiazole-5-carboxamide\|H-Glu-Pro-Gln-Tyr(PO3H2)-Glu-Glu-Ile-Pro-Ile-Tyr-Leu- | |
| | OH\|Glu-Pro-Gln-F2Pmp-Glu-Glu-Ile-Pro-Ile-Tyr-Leu\|O-Phospho-L-tyrosine\|S- | |
| | Hydroxycysteine\|2,6-Diformyl-4-methylphenyl dihydrogen phosphate\|N-Acetyl-N-[1-(1,1′- | |
| | biphenyl-4-ylmethyl)-2-oxoazepan-3-YL]-3-formyl-O-phosphonotyrosinamide\|(2R)-2-phenyl-2- | |
| | sulfoacetic acid\|N6-Benzyl adenosine-5′-diphosphate\|{4-[2-Acetylamino-2-(1-biphenyl-4- | |
| | ylmethyl-2-oxo-azepan-3-ylcarbamoyl)-ethyl]-2-phosphono-phenoxy}-acetic acid\|2-Formylphenyl | |
| | dihydrogen phosphate\|N-Acetyl-N-[1-(1,1′-biphenyl-4-ylmethyl)-2-oxoazepan-3-YL]-3,4- | |
| | diphosphonophenylalaninamide\|2-{4-[2-Acetylamino-2-(1-biphenyl-4-ylmethyl-2-oxo-azepan-3- | |
| | ylcarbamoyl)-ethyl]-2-methoxycarbonyl-phenyl}-2-fluoro-malonic acid\|2-Cyanoquinolin-8-YL | |
| | dihydrogen phosphate\|(Phenyl-phosphono-methyl)-phosphonic acid\|Pasbn\|[4-((1Z)-2- | |
| | (Acetylamino)-3-{[1-(1,1′-biphenyl-4-ylmethyl)-2-oxoazepan-3-YL]amino}-3-oxoprop-1-enyl)-2- | |
| | formylphenyl]acetic acid\|Cyclohexylmethyl 2-formylphenyl hydrogen phosphate\|Hydroxy(1- | |
| | naphthyl)methylphosphonic acid\|4-[2-Acetylamino-2-(1-biphenyl-4-ylmethyl-2-oxo-azepan-3- | |
| | ylcarbamoyl)-ethyl]-2-formyl-benzoic acid\|Phenyl dihydrogen phosphate\|2-Formyl-6- | |
| | methoxyphenyl dihydrogen phosphate\|1,2,3,4-Tetrahydroquinolin-8-YL dihydrogen | |
| | phosphate\|N-Acetyl-N-[1-(1,1′-biphenyl-4-ylmethyl)-2-oxoazepan-3-YL]-4- | |
| | [difluoro(phosphono)methyl]phenylalaninamide\|[(4-Ethylphenyl)amino]carbonylphosphonic | |
| | acid\|5-[2-Acetylamino-2-(1-biphenyl-4-ylmethyl-2-oxo-azepan-3-ylcarbamoyl)-ethyl]-2- | |
| | carboxymethyl-benzoic acid\|Oxalic Acid\|Phenylmalonic acid\|N-Acetyl-N-[1-(1,1′-biphenyl-4- | |
| | ylmethyl)-2-oxoazepan-3-YL]-O-phosphonotyrosinamide\|2-(4-Carcoxy-5- | |
| | isopropylthiazolyl)benzopiperidine\|1-(4-(4-amino-5-(3-methoxyphenyl)-7H-pyrrolo[2,3- | |
| | d]pyrimidin-7-yl)phenethyl)piperidin-4-ol\|1H-Indole-2-carboxamide,N-(4-(1-(trans-4-(4-acetyl-1- | |
| | piperazinyl)cyclohexyl)-4-amino-1H-pyrazolo(3,4-d)pyrimidin-3-yl)-2-methoxyphenyl)-1-methyl- | |
| | IN-(3-fluorophenyl)-6,7-dimethoxy-1,4-dihydroindeno[1,2-c]pyrazol-3-amine\|Y-c[D-Pen-(2S,3R)- | |
| | 2-Me-(2″)Nal-GSFC]KR-NH2\|Y-c[D-Pen-(2R,3R)-2-Me-(2″)Nal-GSFC]KR-NH2\|Y-c[D-Pen-(3- | |
| | I)Tyr-GSFC]KR-NH2\|Y-c[D-Pen-(3,5-dil)Tyr-GSFC]KR-NH2\|Y-c[D-Pen-(2S,3S)-2-Me-(2″)Nal- | |
| | GSFC]KR-NH2\|Y-c[D-Pen-(2″)Nal-GSFC]KR-NH2\|Y-c[D-Pen-D-(2″)Nal-GSFC]KR-NH2\|Y-c[D- | |
| | Pen-(2R,3S)-2-Me-(2″)Nal-GSFC]KR-NH2\|N-(4-{4-Amino-1-[4-(4-Methylpiperazin-1-Yl)-Trans- | |
| | Cyclohexyl]-1h-Pyrazolo[3,4-D]pyrimidin-3-Yl}-2-Methoxyphenyl)-1-Methyl-1h-Indole-2- | |
| | Carboxamide\|Unii-3mae0mxi6K\|N,5-Diphenylimidazo[1,5-A]pyrazin-8-amine\|Imidazo(1,5- | |
| | a)pyrido(3,2-E)pyrazin-6-amine,N-(2-chloro-6-methylphenyl)-2-((3S)-3-methyl-1-piperazinyl)-\|7- | |
| | (Naphthalen-2-yl)benzo[e][1,2,4]triazin-3-amine\|3-(3-Aminobenzo[e][1,2,4]triazin-7-yl)phenol\|6- | |
| | (2,6-dichlorophenyl)-8-methyl-2-((3-(methylthio)phenyl)amino)pyrido[2,3-d]pyrimidin-7(8H)- | |
| | one\|4-((5-(3-Chloro-4-methylphenyl)furan-2-yl)methylene)-1-(3,4-dimethylphenyl)pyrazolidine- | |
| | 3,5-dione\|4-(3-(Benzyloxy)benzylidene)-1-(3,4-dichlorophenyl)pyrazolidine-3,5-dione\|(4Z)-4-[(6- | |
| | bromo-1,3-benzodioxol-5-yl)methylidene]-1-(4-iodophenyl)pyrazolidine-3,5-dione\|4-(4- | |
| | (Diethylamino)-2-hydroxybenzylidene)-1-(4-iodophenyl)pyrazolidine-3,5-dione\|4-(4- | |
| | Iodobenzylidene)-1-(4-iodophenyl)pyrazolidine-3,5-dione\|4-Benzylidene-1-(4-iodophenyl)-3,5- | |
| | pyrazolidinedione\|4-(2-(Benzyloxy)benzylidene)-1-phenylpyrazolidine-3,5-dione\|N-[5-(1H-indol- | |
| | 3-ylmethyl)-1,3,4-thiadiazol-2-yl]-4-methoxybenzamide\|2-methoxy-4-(methylthio)-N-(5- | |
| | phenethyl-1,3,4-thiadiazol-2-yl)benzamide\|2,4-dichloro-N-(5-phenethyl-1,3,4-thiadiazol-2- | |
| | yl)benzamide\|2-iodo-N-(5-phenethyl-1,3,4-thiadiazol-2-yl)benzamide\|N-(5-phenethyl-1,3,4- | |
| | thiadiazol-2-yl)benzamide\|H-Ala-Gly-Cys(1)-Lys-D-Asn-Phe-D-Phe-Trp-D-Lys-D-xiThr-Ala(Unk)- | |
| | D-xiThr-Ser-Cys(1)-OH\|Cyclo[Ac-Cys-Ile-Tyr-Lys-Tyr-Phe]\|(2S)-2-[(2S)-2-[(2S)-6-amino-2- | |
| | [[(8S,11S,14S)-11-butan-2-yl-3,6,9,12-tetraoxo-8-(sulfanylmethyl)-2,7,10,13- | |
| | tetrazabicyclo[13.2.2]nonadeca-1(17),15,18-triene-14-carbonyl]amino]hexanoyl]amino]-3-(4- | |
| | hydroxyphenyl)propanoyl]amino]-3-(4-hydroxyphenyl)propanoic acid\|Cyclo[Ac-Cys-Ile-Tyr-Lys- | |
| | Tyr-Tyr]\|Ac-Cys-xille-Tyr-Lys-Phe(4-N3)-Tyr-OH\|Ac-Cys-xille-Tyr-Lys-Phe(4-CN)-Tyr-OH\|Ac- | |
| | Cys-xille-Tyr-Lys-Phe(4-1)-Tyr-OH\|Ac-Cys-xille-Tyr-Lys-Phe(4-NO2)-Tyr-Phe-OH\|Ac-Cys-xille- | |
| | Phe(4-NO2)-Lys-Tyr-Phe(4-NO2)-OH\|Ac-Cys-xille-Tyr-Lys-Phe(4-NO2)-Phe(4-NO2)-OH\|Ac- | |
| | Cys-xille-Phe(4-NO2)-Lys-Phe(4-NO2)-Tyr-OH\|Ac-Cys-xille-Tyr-Lys-Tyr-Phe(4-NO2)-OH\|Ac- | |
| | Cys-xille-Phe(4-NO2)-Lys-Tyr-Tyr-OH\|Ac-Cys-xille-Tyr-Lys-Phe(4-Cl)-Tyr-OH\|6-(2,6- | |
| | Dichlorophenyl)-2-{[3-(hydroxymethyl)phenyl]amino}-8-methylpyrido[2,3-D]pyrimidin-7(8H)- | |
| | one\|6-(2,6-Dichlorophenyl)-8-methyl-2-(4-morpholin-4-ylanilino)pyrido[2,3-d]pyrimidin-7- | |
| | one\|Pyrido(2,3-d)pyrimidin-7(8H)-one,6-(2,6-dichlorophenyl)-2-((4-fluorophenyl)amino)-8- | |
| | methyl-\|2-[(4-Ethoxyphenyl)amino]-8-methyl-6-(2,6-dichlorophenyl)pyrido[2,3-d]pyrimidine- | |
| | 7(8H)-one\|2-[4-[2-[Ethyl(acetyl)amino]ethoxy]phenyl]amino]-8-methyl-6-(2,6- | |
| | dichlorophenyl)pyrido[2,3-d]pyrimidine-7(8H)-one\|6-(2,6-Dichlorophenyl)-2-[(4-Fluoro-3- | |
| | Methylphenyl)amino]-8-Methylpyrido[2,3-D]pyrimidin-7(8h)-One\|(3z)-N,N-Dimethyl-2-Oxo-3- | |
| | (4,5,6,7-Tetrahydro-1h-Indol-2-Ylmethylidene)-2,3-Dihydro-1h-Indole-5-Sulfonamide\|1-tert-butyl- | |
| | 3-(naphthalen-1-ylmethyl)-1H-pyrazolo[3,4-d]pyrimidin-4-amine\|Benzamide,3-((1E)-2-(6- | |
| | (cyclopropylamino)-9H-purin-9-yl)ethenyl)-4-methyl-N-(4-(trifluoromethyl)-2-pyridinyl)-\|3-{(E)-2- | |
| | [6-(Cyclopropylamino)-9h-Purin-9-Yl]ethenyl}-4-Methyl-N-[3-(4-Methyl-1h-Imidazol-1-Yl)-5- | |
| | (Trifluoromethyl)phenyl]benzamide\|4-[(2,4-Dichloro-5-methoxyphenyl)amino]-6-methoxy-7-[4-(4- | |
| | methylpiperazin-1-yl)but-1-ynyl]-3-quinolinecarbonitrile\|5,7-Diphenylpyrrolo(2,3-d)pyrimidin-4- | |
| | amine\|5,7-Diphenyl-7H-pyrrolo[2,3-d]pyrimidin-4-ol\|1,3-diphenyl-1H-pyrazolo[3,4-d]pyrimidin-4- | |
| | amine\|4-Chloro-5,7-diphenyl-7H-pyrrolo[2,3-d]pyrimidine\|N-{4-[(3- | |
| | Bromophenyl)amino]quinazolin-6-Yl}propanamide\|{4-[2-Acetylamino-2-(3-carbamoyl-2- | |
| | cyclohexylmethoxy-6,7,8,9-tetrahydro-5H-benzocyclohepten-5ylcarbamoyl)-ethyl]-2-phosphono- | |
| | phenyl}-phosphonic acid\|N-(3-fluorobenzyl)-2-(5-(4-morpholinophenyl)pyridin-2-yl)acetamide\|(+/−)- | |
| | 2-(2-Cinnamamidobenzamido)-3-phenylpropanoic acid\|Penicilliol A\|Csf1R-IN-2\|Methyl | |
| | Pyridazine-4-carboxylate\|Saracatinib\|Anlotinib\|Crenolanib\|Masitinib\|7-Fluoro-3-[(2-Fluoro-4- | |
| | Iodophenyl) amino]-N-{[(2s)-2-Hydroxypropyl]oxy}furo[3,2-C]pyridine-2-Carboxamide\|Herbimycin | |
| | a\|Bosutinib\|4-[(2,4-Dichloro-5-methoxyphenyl)amino]-6-methoxy-7-{5-[(4-methylpiperazin-1- | |
| | yl)methyl]furan-3-yl}quinoline-3-carbonitrile\|Dasatinib\|6-(2,6-Dichlorophenyl)-2-[4-[2- | |
| | (diethylamino)ethoxy]anilino]-8-methylpyrido[2,3-d]pyrimidin-7-one\|N-[4-chloro-3-[2-[4-(4- | |
| | methylpiperazin-1-yl)anilino]-5-oxo-7,8-dihydropyrido[4,3-d]pyrimidin-6-yl]phenyl]-3- | |
| | (trifluoromethyl)benzamide\|Unii-zsa4DW7Idj\|4-(4-Chloro-2-cyclopropyl-5-methoxyanilino)-7-[3- | |
| | [4-(deuteriomethyl)piperazin-1-yl]propoxy]-6-methoxyquinoline-3-carbonitrile\|3-(2- | |
| | Hydroxyethylcarbamoyl)pyrido[1,2-a]indole-10-carboxylic acid\|8-(1,3-oxazol-5-yl)-N-phenyl-6- | |
| | (trifluoromethyl)imidazo[1,2-a]pyridine-3-carboxamide\|Topaquinone\|2-(3-Chloro-phenyl)-1H- | |
| | [1,8]naphthyridin-4-one\|(3R,5R,6E)-7-[4,5-bis(4-fluorophenyl)-2-(propan-2-yl)-1H-imidazol-1-yl]- | |
| | 3,5-dihydroxyhept-6-enoic acid\|H-DL-Tyr(2,6-diMe)-DL-Tic-DL-Lys(Cbz)-NHPh | |
| SPHK1 | 4-[4-(4-Chlorophenyl)-2-thiazolyl]amino]phenol\|(2R,3S,4E)-2-(methylamino)-5-(4- | Inhibitor\| |
| | pentylphenyl)pent-4-ene-1,3-diol\|{(2R)-1-[4-({3-Methyl-5- | Modulator |
| | [(phenylsulfonyl)methyl]phenoxy}methyl)benzyl]pyrrolidin-2-YL}methanol\|Pyridoxamine\|CID | |
| | 9862964\|Fingolimod hydrochloride\|Idronoxil\|1-[2-(4-Octylphenyl)ethyl]piperidin-4-amine\|[(2R)-1- | |
| | [2-(4-dodecylphenyl)ethyl]pyrrolidin-2-yl]methanol\|3-{4-[6-(5,5,8,8-Tetramethyl-5,6,7,8- | |
| | tetrahydronaphthalen-2-yl)pyridin-2-yl]-piperazin-1-yl}propan-1-ol\|Tetrahydroquinoline A\|[(2R)-1- | |
| | [[5-[3-(cyclopentyloxymethyl)phenyl]-1H-indol-2-yl]methyl]pyrrolidin-2-yl]methanol\|CID | |
| | 46911228\|2-[3-[2-[(2R)-2-(hydroxymethyl)pyrrolidin-1-yl]methyl]-1H-indol-5-yl]phenyl]-4,4- | |
| | dimethylcyclohex-2-en-1-one\|[(2R)-1-[5-[3-(cyclohexen-1-yl)phenyl]-1H-indol-2- | |
| | yl]methyl]pyrrolidin-2-yl]methanol\|[(2R)-1-[5-(3-tert-butylphenyl)-1H-indol-2-yl]methyl]pyrrolidin- | |
| | 2-yl]methanol\|4-[4-(5,5,8,8-Tetramethyl-6,7-dihydronaphthalen-2-yl)pyrimidin-2- | |
| | yl]benzenecarboximidamide\|(2S,3S)-N-(4-(5-heptyl-1,2,4-oxadiazol-3-yl)benzyl)-3- | |
| | hydroxypyrrolidine-2-carboxamide | |
| BRD2 | Carbamic acid,(1-methyl-6-phenyl-4H-[1,2,4]triazolo[4,3-a][1,4]benzodiazepin-4-yl)- | Inhibitor\| |
| | phenylmethyl ester (9CI)\|7-(3,5-Dimethylisoxazol-4-yl)-8-methoxy-1-((R)-1-(pyridin-2-yl)ethyl)- | Modulator |
| | 1H-imidazo[4,5-c]quinolin-2(3H)-one\|Methyl (2r)-2-[(4s)-6-(4-Chlorophenyl)-8-Methoxy-1-Methyl- | |
| | 4h-[1,2,4]triazolo[4,3-A][1,4]benzodiazepin-4-Yl]butanoate\|Methyl (2r)-2-[(4s)-6-(4- | |
| | Chlorophenyl)-8-Methoxy-1-Methyl-4h-[1,2,4]triazolo[4,3-A][1,4]benzodiazepin-4- | |
| | Yl]propanoate\|4-acetyl-N-[5-(diethylsulfamoyl)-2-hydroxyphenyl]-3-ethyl-5-methyl-1H-pyrrole-2- | |
| | carboxamide\|1-[2-(1H-benzimidazol-2-ylsulfanyl)ethyl]-3-methyl-1,3-dihydro-2H-benzimidazole- | |
| | 2-thione\|3-(dimethyl-1,2-oxazol-4-yl)-2-methoxy-9-[2-(trifluoromethoxy)phenyl]-7H,8H,9H- | |
| | pyrrolo[2,3-c]1,5-naphthyridin-8-one\|9-benzyl-3-(dimethyl-1,2-oxazol-4-yl)-2-methoxy-7H,8H,9H- | |
| | pyrrolo[2,3-c]1,5-naphthyridin-8-one\|1-[3-(Dimethyl-1,2-oxazol-4-yl)-5-ethoxyphenyl]ethan-1- | |
| | ol\|N-ethyl-4-[2-(4-fluoro-2,6-dimethylphenoxy)-5-(2-hydroxypropan-2-yl)phenyl]-6-methyl-7-oxo- | |
| | 1H-pyrrolo[2,3-c]pyridine-2-carboxamide\|Birabresib\|U.S. Pat No. 9,550,796, Example 17\|N-[3-hydroxy-5- | |
| | [(R)-hydroxy(phenyl)methyl]phenyl]-N-methylprop-2-enamide\|N-[3-hydroxy-5- | |
| | [hydroxy(phenyl)methyl]phenyl]-N-methylprop-2-enamide\|N-[3-hydroxy-5-[(S)- | |
| | hydroxy(phenyl)methyl]phenyl]-N-methylprop-2-enamide\|[3-[Hydroxy(phenyl)methyl]-5- | |
| | [methyl(prop-2-enoyl)amino]phenyl]2,2-dimethylpropanoate | |
| KC1D | 2-[2-(4-fluoro-2-methylphenoxymethyl)-5-methoxypyridin-4-yl]-1H,4H,5H,6H,7H-pyrrolo[3,2- | Inhibitor |
| | c]pyridin-4-one\|3-[(2,4,6-Trimethoxy-phenyl)-methylene]-indolin-2-one\|3-[(3- | |
| | Chlorophenoxy)methyl]-1-(Tetrahydro-2h-Pyran-4-Yl)-1h-Pyrazolo[3,4-D]pyrimidin-4-Amine\|CK1 | |
| | Inhibitor\|PF-670462 | |
| AURKA | Phosphonothreonine\|2-(1H-pyrazol-3-yl)-1H-benzimidazole\|Indirubin-3-methoxime\|Indirubin-3′- | Inhibitor\| |
| | monoxime\|6-Bromoindirubin-3′-oxime\|Indirubin-3-acetoxime\|#N/A\|N-[4-[6-Methoxy-7-[3-(4- | Modulator |
| | morpholinyl)propoxy]-4-quinazolinyl]amino]phenyl]benzamide\|4-(1H-pyrazol-4-yl)-1H-pyrrolo[2,3- | |
| | b]pyridine\|N-(2-((1S,4R)-6-((4-(Cyclobutylamino)-5-(trifluoromethyl)pyrimidin-2-yl)amino)-1,2,3,4- | |
| | tetrahydro-1,4-epiminonaphthalen-9-yl)-2-oxoethyl)acetamide\|Sns-314\|MK-5108\|4-[9-Chloro-7- | |
| | (2,6-difluorophenyl)-5H-pyrimido[5,4-d][2]benzazepin-2-yl]amino]benzoic | |
| | acid\|Cenisertib\|Angiotensin IV\|4-Methyl-5-(2-(4-morpholinophenylamino)pyrimidin-4-yl)thiazol-2- | |
| | amine\|N-(4-(3-(2-aminopyrimidin-4-yl)pyridin-2-yloxy)phenyl)-4-(4-methylthiophen-2- | |
| | yl)phthalazin-1-amine\|Sulfadiazine\|Simufilam\|(2R,4R)-1-(3-Chloro-2-fluorobenzyl)-4-((3-fluoro-6- | |
| | ((5-methyl-1H-pyrazol-3-yl)amino)pyridin-2-yl)methyl)-2-methylpiperidine-4-carboxylic acid\|(E)- | |
| | N-(5-Methyl-1H-pyrazol-3-yl)-6-(4-methylpiperazin-1-yl)-2-styrylpyrimidin-4- | |
| | amine\|Danusertib\|Tozasertib\|Ilorasertib\|Alisertib\|1-Cyclopropyl-3-(3-(5-(morpholinomethyl)-1H- | |
| | benzo[d]imidazol-2-yl)-1H-pyrazol-4-yl)urea\|Orantinib | |
| ROR1 | Lintopride\|4-(3-(4-Hydroxybutyl)-4,4-dimethyl-2,5-dioxoimidazolidin-1-yl)-2- | CAR-T-Cell- |
| | (trifluoromethyl)benzonitrile\|Sibofimloc\|2,3-Diphenyl-1H-indole\|Zeteletinib hemiadipate | Therapy\|.\| |
| | | Antagonist |
| IRAK3 | N-[(3R)-8-[5-chloro-4-[2-fluoro-6-(methylcarbamoyl)anilino]pyrimidin-2-yl]amino]-5,5-dimethyl-2- | Inhibitor |
| | oxo-3,4-dihydro-1H-1-benzazepin-3-yl]pyrrolidine-1-carboxamide | |
| ARAF | (3R)-3-hydroxy-3-methyl-5-(phosphonooxy)pentanoic acid\|2-(4-Methylphenyl)-1H- | .\|Inhibitor |
| | anthraceno[1,2-d]imidazole-6,11-dione\|Leucovorin\|Esuberaprost Sodium\|\|gernellin\|MK- | |
| | 2461\|Fasiglifam\|2-(6H-benzo[b][1,4]benzodioxepin-3-yl)acetic acid\|1H-Imidazole,1-[2-(4- | |
| | chlorophenyl)ethyl]-\|Trifenagrel\|4-Benzoyl-I-phenylalanine\|Phenanthrene-3-carboxylic acid | |
| | hydroxyamide\|4-(4-Chlorophenylsulfonylcarbamoyl)benzoyl-valyl-prolyl-1-(1-trifluoroacetyl-2- | |
| | methylprolyl)amide\|Cki27; CK127; CK 127; RG-7304; RG 7304 | |
| M3K10 | 3-(1H-indol-5-yl)-5-(4-((4-methylpiperazin-1-yl)methyl)phenyl)-1H-pyrrolo[2,3- | Inhibitor |
| | b]pyridine\|Itraconazole\|2-Isopropoxy-12-(2-hydroxyethyl)-13,14-dihydronaphthol[2,1- | |
| | a]pyrrolo[3,4-c]carbazole-5-one\|2-Methoxy-12-(2-hydroxyethyl)-13,14-dihydronaphthol[2,1- | |
| | a]pyrrolo[3,4-c]carbazole-5-one | |
| CDK6 | Cdk/Crk Inhibitor\|Fisetin\|Deschloroflavopiridol\|Fascaplysine\|Apigenin\|Chrysin\|1H-Pyrrole-2,5- | Inhibitor\| |
| | dione,3,4-bis(4-methoxyphenyl)-\|3,4-diphenyl-1H-pyrrole-2,5-dione\|2-(4-Methoxyphenyl)-3- | Modulator |
| | phenylmaleimide\|1-(1-Phenyl-2-(pyridin-2-yl)ethyl)piperazine\|8-Bicyclo[2.2.1]hept-2-yl-2-{4-[4-(3- | |
| | hydroxypropyl)piperidin-1-yl]phenylamino}-8H-pyrido[2,3-d]pyrimidin-7-one\|2-({2-[(R)-2-Amino-3- | |
| | (4-hydroxy-2,6-dimethyl-phenyl)-propionyl]-1,2,3,4-tetrahydro-isoquinoline-3-carbonyl}-amino)- | |
| | propionic acid\|Zidovudine\|N-[4-[(4-methylpiperazin-1-yl)methyl]phenyl]-4-(7H-pyrrolo[2,3- | |
| | d]pyrimidin-4-ylamino)-1H-pyrazole-5-carboxamide\|Unii-uch3qqh1C0\|Ceftobiprole medocaril free | |
| | acid\|Lerociclib\|Ribociclib\|Palbociclib\|Trilaciclib\|Apremilast\|Abemaciclib\|Ribociclib succinate\|4-[4- | |
| | [2-[5-[(5-tert-butyl-1,3-oxazol-2-yl)methylsulfanyl]-1,3-thiazol-2-yl]amino]-2-oxoethyl]phenoxy]-N- | |
| | hydroxybutanamide\|4-((1R,2R)-2-Hydroxycyclohexyl)-2(trifluoromethyl)benzonitrile\|4-[3- | |
| | Hydroxyanilino]-6,7-dimethoxyquinazoline\|N-(4-(Diethylamino)benzyl)-4-methoxy-N-(p- | |
| | tolyl)benzenesulfonamide\|(2,5-Dimethoxybenzyl)(2-phenylethyl)amine\|N-(9-chloro-2-(furan-2-yl)- | |
| | [1,2,4]triazolo[1,5-c]quinazolin-5-yl)-2-phenylacetamide\|Cpzen-45 | |
| SBK3 | N-[(3R)-8-[5-chloro-4-[2-fluoro-6-(methylcarbamoyl)anilino]pyrimidin-2-yl]amino]-5,5-dimethyl-2- | Inhibitor |
| | oxo-3,4-dihydro-1H-1-benzazepin-3-yl]pyrrolidine-1-carboxamide | |
| MARK3 | N-{3-[(5-cyclopropyl-2-{[4-(morpholin-4-yl)phenyl]amino}pyrimidin-4- | Inhibitor |
| | yl)amino]propyl}cyclobutanecarboxamide\|Cdc25C phosphatase (211-221) | |
| BRAF | N-(3-(5-chloro-1H-pyrrolo[2,3-b]pyridine-3-carbonyl)-2,4-difluorophenyl)propane-1- | Inhibitor\|.\| |
| | sulfonamide\|Dadle\|Caged vemurafenib prodrug\|Prinaberel\|Elamipretide\|syn-BrP-LPAJERK | Modulator |
| | Inhibitor III\|(1S,2S,3R,4R)-3-((5-chloro-2-(((S)-1-methoxy-7-morpholino-6,7,8,9-tetrahydro-5H- | |
| | benzo[7]annulen-2-yl)amino)pyrimidin-4-yl)amino)bicyclo[2.2.1]hept-5-ene-2- | |
| | carboxamide.\|U.S. Pat No. 9,550,796, Example 54\|3-(dimethylamino)-N-(3-(4-hydroxybenzamido)-4- | |
| | methylphenyl)benzamide\|2-chloro-5-(2-phenyl-5-(pyridin-4-yl)-1H-imidazol-4-yl)phenol\|2- | |
| | (Benzylamino)-6-(3-acetamidophenyl)pyrazine\|2-(Phenylamino)-6-(3- | |
| | acetamidophenyl)pyrazine\|Ecopladib\|Amphotericin b\|Cki27; CK127; CK 127; RG-7304; RG | |
| | 7304\|(r)-2-(1-(6-Amino-5-chloropyrimidine-4-carboxamido)ethyl)-n-(5-chloro-4- | |
| | (trifluoromethyl)pyridin-2-yl)thiazole-5-carboxamide\|2-[4-[(1Z)-1-hydroxyimino-2,3-dihydroinden- | |
| | 5-yl]-3-pyridin-4-ylpyrazol-1-yl]ethanol\|3-(2-Cyanopropan-2-yl)-N-(4-methyl-3-((3-methyl-4-oxo- | |
| | 3,4-dihydroquinazolin-6-yl)amino)phenyl)benzamide\|MK-0773\|BMS-908662 free base\|2- | |
| | Phenylethynyl-5,6,7,8-tetrahydro-quinoline\|Arq 736[who-DD]\|(2R)-N-[(2R)-3,3-dimethyl-1- | |
| | (methylamino)-1-oxobutan-2-yl]-N′-hydroxy-2-(2-methylpropyl)butanediamide\|Mgb-BP- | |
| | 3\|Beigene-283\|Agerafenib\|(R)-N-(3-(5-(2-cyclopropylpyrimidin-5-yl)-1H-pyrrolo[2,3-b]pyridine-3- | |
| | carbonyl)-2,4-difluorophenyl)-3-fluoropyrrolidine-1- | |
| | sulfonamide\|Dabrafenib\|Vemurafenib\|Encorafenib | |
| BMP2K | [4-[1-[5-chloro-2-(trifluoromethyl)phenyl]methyl]-3,4-dihydro-2H-pyrido[2,3-b]pyrazin-7- | Inhibitor |
| | yl]phenyl]-(4-pyrrolidin-1-ylpiperidin-1-yl)methanone | |
| BRDT | 4-acetyl-N-[5-(diethylsulfamoyl)-2-hydroxyphenyl]-3-ethyl-5-methyl-1H-pyrrole-2- | Inhibitor |
| | carboxamide\|(S)-(+)-tert-Butyl 2-(4-(4-chlorophenyl)-2,3,9-trimethyl-6H-thieno(3,2- | |
| | f)(1,2,4)triazolo(4,3-a)(1,4)diazepin-6-yl)acetate\|N-[3-hydroxy-5-[(R)- | |
| | hydroxy(phenyl)methyl]phenyl]-N-methylprop-2-enamide\|N-[3-hydroxy-5- | |
| | [hydroxy(phenyl)methyl]phenyl]-N-methylprop-2-enamide\|N-[3-hydroxy-5-[(S)- | |
| | hydroxy(phenyl)methyl]phenyl]-N-methylprop-2-enamide\|[3-[Hydroxy(phenyl)methyl]-5- | |
| | [methyl(prop-2-enoyl)amino]phenyl]2,2-dimethylpropanoate | |
| FGFR3 | Trapidil\|Cyclopropanolamide,12a\|Actb-1003\|(S)-5-fluoro-2-(1-(5-fluoropyridin-2-yl)ethylamino)- | Inhibitor\|.\| |
| | 6-(5-methyl-1H-pyrazol-3-ylamino)nicotinonitrile\|3-[(3-(2-Carboxyethyl)-4-methylpyrrol-2- | Antagonist |
| | YL)methylene]-2-indolinone\|5,11-dimethyl-6H-pyrido[4,3-b]carbazol-9-ol\|7-(4-fluoroanilino)-1- | |
| | [(1R,3R)-3-hydroxycyclopentyl]-3-(4-methoxyphenyl)-4H-pyrimido[4,5-d]pyrimidin-2-one\|8- | |
| | Bicyclo[2.2.1]hept-2-yl-2-{4-[4-(3-hydroxypropyl)piperidin-1-yl]phenylamino}-8H-pyrido[2,3- | |
| | d]pyrimidin-7-one\|(E)-4-(5-ethoxypyridin-3-yl)-N-methylbut-3-en-1-amine\|MK-2461 \|(R)-6-(4-((4- | |
| | Ethylpiperazin-1-yl)methyl)phenyl)-N-(1-phenylethyl)-7H-pyrrolo[2,3-d]pyrimidin-4-amine\|Debio- | |
| | 1347\|Phosphorodiamidic acid,N,N-bis(2-chloroethyl)-N′-(3-hydroxypropyl)-,(3-chloropropyl) | |
| | ester\|Tofisopam\|Dovitinib lactate\|Brivanib alaninate\|Lucitanib\|Pemigatinib | |
| ROR2 | (3R,5S,6E)-3,5-Dihydroxy-7-[1-phenyl-2-isopropyl-4-(4-fluorophenyl)-1H-pyrrol-3-yl]-6-heptenoic | CAR-T-Cell- |
| | acid\|3,4-Dimethoxy-N-[6-(4-Methoxyphenoxy)-1,3-Dimethyl-2-Oxo-2,3-Dihydro-1h- | Therapy |
| | Benzimidazol-5-Yl]benzenesulfonamide | |
| TNK1 | 2-{[2-ethoxy-4-(4-hydroxypiperidin-1-yl)phenyl]amino}-5,11-dimethyl-5,11-dihydro-6H- | Inhibitor |
| | pyrimido[4,5-b][1,4]benzodiazepin-6-one | |
| ADK | Adenosine kinase inhibitor GP515\|Propanamide,N-(4,6-diphenyl-2-pyrimidinyl)-2-methyl-\|5′- | Inhibitor |
| | Iodotubercidin\|(3R,4S,5R)-2-(4-amino-5-iodopyrrolo[2,3-d]pyrimidin-7-yl)-5- | |
| | (hydroxymethyl)oxolane-3,4-diol\|7-(5-Deoxy-Beta-D-Ribofuranosyl)-5-Iodo-7h-Pyrrolo[2,3- | |
| | D]pyrimidin-4-Amine\|6-Benzylthioinosine\|(1S,2R,3S,5R)-3-amino-5-{4-amino-5-iodo-7H- | |
| | pyrrolo[2,3-d]pyrimidin-7-yl}cyclopentane-1,2-diol\|2-Methyl-5-(5-phenyl-4-phenylamino- | |
| | pyrrolo[2,3-d]pyrimidin-7-yl)-tetrahydro-furan-3,4-diol\|Abt-702\|Udenafil | |
| ITK | N-(5-(3-(1-acetylpiperazine-4-carbonyl)-4,5-dimethylbenzylthio)thiazol-2-yl)-4-((3,3- | Inhibitor |
| | dimethylbutan-2-ylamino)methyl)benzamide\|Benzamide,N-(5-((5-((4-acetyl-1- | |
| | piperazinyl)carbonyl)-4-methoxy-2-methylphenyl)thio)-2-thiazolyl)-4-(((1,2,2- | |
| | trimethylpropyl)amino)methyl)-[N-[1-[(2R,4R,5R)-5-(hydroxymethyl)-4-prop-2-ynoxyoxolan-2-yl]- | |
| | 2-oxopyrimidin-4-yl]benzamide\|Neorautenol\|(5-(3-methoxyphenyl)-1-pentyl-1H-pyrrol-3- | |
| | yl)(naphthalen-1-yl)methanone\|3-Hydroxybutan-2-yl 6,11-dimethyl-6,11- | |
| | diazatetracyclo[7.6.1.0\hat{ }{2,7}.0\hat{ }{12,16}]hexadeca-1(16),9,12,14-tetraene-4-carboxylate | |
| TIE2 | Bis(p-bromophenyl)sulfone\|5-((5-(4-((4-(2-hydroxyethyl)piperazin-1-yl)methyl)-3- | Modulator\| |
| | (trifluoromethyl)phenylcarbamoyl)-2-methylphenyl)ethynyl)-N,1-dimethyl-1H-imidazole-2- | Inhibitor\| |
| | carboxamide\|N-(4-phenoxyphenyl)quinazolin-4-amine\|N-(4-{4-Amino-1-[4-(4-Methylpiperazin-1- | Activator |
| | Yl)-Trans-Cyclohexyl]-1h-Pyrazolo[3,4-D]pyrimidin-3-Yl}-2-Methoxyphenyl)-1-Methyl-1h-Indole- | |
| | 2-Carboxamide\|2-(4-Methoxyphenyl)-3-phenylmaleimide\|2-(1H-Indole-3-yl)-3- | |
| | phenylmaleimide\|1H-Pyrrole-2,5-dione,3,4-bis(4-methoxyphenyl)-\|3,4-diphenyl-1H-pyrrole-2,5- | |
| | dione\|Mitoquinone\|Pexmetinib\|Altiratinib\|Cep-11981\|Rebastinib\|Razuprotafib | |
| RIPK2 | Src Kinase Inhibitor I\|5-Methyl-4-propan-2-ylbenzene-1,3-diol\|Thiourea,N-(5-chloro-2-pyridinyl)- | Inhibitor\|. |
| | N′-(2-(3-cyano-2,6-difluorophenyl)ethyl)-\|Hadacidin\|U.S. Pat No. 9,550,796, Example 98\|Econazole | |
| | nitrate\|L-Homoarginine\|Phenylacetylrinvanil\|1-tert-butyl-3-(naphthalen-1-ylmethyl)-1H- | |
| | pyrazolo[3,4-d]pyrimidin-4-amine\|\|(+/−)-2-(2-Cinnamamidobenzamido)-3-phenylpropanoic acid | |
| EF2K | Lapatinib | Inhibitor |
| DYR1A | Benzimidazole\|(Z)-1-(3-Ethyl-5-methoxy-2,3-dihydrobenzothiazol-2-ylidene)propan-2- | Inhibitor |
| | one\|Harmine\|Ethyl 3-[(E)-2-Amino-1-Cyanoethenyl]-6,7-Dichloro-1-Methyl-1h-Indole-2- | |
| | Carboxylate\|leucettine L41\|5-(benzo[d][1,3]dioxol-5-yl)-N-(3,5-dichlorobenzyl)pyrimidin-4- | |
| | amine\|Methyl 9-(4-bromo-2-fluoroanilino)-[1,3]thiazolo[5,4-f]quinazoline-2-carboximidate\|Methyl | |
| | 9-(4-chloroanilino)-[1,3]thiazolo[5,4-f]quinazoline-2-carboximidate\|4-(1,3-benzodioxol-5-yl)-N- | |
| | [(3,5-dichlorophenyl)methyl]pyrimidin-2-amine\|leucettamine B\|(E)-3-Tosylacrylonitrile\|Slotoxin\|N- | |
| | (5-Methoxy-1,3-Benzothiazol-2-Yl)ethanamide\|N-(6-nitro-1,3-benzothiazol-2-yl)acetamide\|N-(5- | |
| | Oxidanyl-1,3-Benzothiazol-2-Yl)ethanamide\|1-[2-(4-Hydroxyphenyl)pyrido[3,2-d]pyrimidin-7- | |
| | yl]piperidin-2-one\|6-(1,3-benzodioxol-5-yl)-N-(thiophen-2-ylmethyl)-4-quinazolinamine\|6- | |
| | (benzo[d][1,3]dioxol-5-yl)-N-neopentylquinazolin-4-amine\|Dgat1-IN-3\|Paprotrain\|5-[5-(piperidin- | |
| | 1-ylmethyl)pyridin-3-yl]-N-(6-propan-2-yloxypyridin-3-yl)-1H-indazole-3-carboxamide\|3-(4- | |
| | Hydroxyphenyl)-5-(3,4-dihydroxyphenyl)-1H-pyrrolo[2,3-b]pyridine\|N-(5-methylpyridin-3-yl)-5-[5- | |
| | (piperidin-1-ylmethyl)pyridin-3-yl]-1H-indazole-3-carboxamide\|N-(6-methylpyridin-3-yl)-5-[5- | |
| | (piperidin-1-ylmethyl)pyridin-3-yl]-1H-indazole-3-carboxamide\|5-[5-(piperidin-1-ylmethyl)pyridin- | |
| | 3-yl]-N-pyridin-3-yl-1H-indazole-3-carboxamide\|11H-pyrido[4,3-a]carbazole-6- | |
| | carbonitrile\|Dcdpc\|Nabumetone\|3-(2,7-Dimethoxyacridin-9-ylthio)propan-1- | |
| | amine\|Silymarin\|#N/A\|Lanperisone\|2,3-Diphenyl-1H-indole\|2-[(4-{2-[(4- | |
| | Cyclohexylbutyl)(cyclohexylcarbamoyl)amino]ethyl}phenyl) ulfanyl]-2-methylpropanoic | |
| | acid\|4,5,6,7-Tetrabromo-N,N-dimethyl-1H-benzimidazol-2-amine\|Bicyclo(2.2.2)octane-1- | |
| | carboxylic acid,4-((((3′R,4′S,5′R)-6″-chloro-4′-(3-chloro-2-fluorophenyl)-1′-ethyl-1″,2″-dihydro-2″- | |
| | oxodispiro(cyclohexane-1,2′-pyrrolidine-3′,3″-(3H)indol)-5′-yl)carbonyl)amino)-\|Methyl 9-((2- | |
| | fluoro-4-methoxyphenyl)amino)thiazolo[5,4-f]quinazoline-2-carbimidate\|Methyl 9-(2,4- | |
| | dichloroanilino)-[1,3]thiazolo[5,4-f]quinazoline-2-carboximidate\|9-methoxy-11H-pyrido[4,3- | |
| | a]carbazole-6-carbonitrile\|1,3,4(2H)-Isoquinolinetrione,2-(2-propenyl)-\|3-amino-N-(5-phenyl- | |
| | 1,3,4-thiadiazol-2-yl)-6,7,8,9-tetrahydro-5H-cyclohepta[b]thieno[3,2-e]pyridine-2- | |
| | carboxamide\|1,9-dimethoxy-11H-pyrido[4,3-a]carbazole-6-carbonitrile\|Methyl 9-(4- | |
| | methylanilino)-[1,3]thiazolo[5,4-f]quinazoline-2-carboximidate\|N-butyl-2-(1H-indol-3-yl)-2- | |
| | oxoacetamide\|Methyl 9-(4-chloro-2-fluoroanilino)-[1,3]thiazolo[5,4-f]quinazoline-2- | |
| | carboximidate\|Methyl 9-(2,4-difluoroanilino)-[1,3]thiazolo[5,4-f]quinazoline-2-carboximidate\|(1z)- | |
| | 1-(3-Ethyl-5-Hydroxy-1,3-Benzothiazol-2(3h)-Ylidene)propan-2-One\|Vyxeos | |
| PKN2 | (3S,4R)-N-(7-chloro-1-oxo-1,2-dihydroisoquinolin-6-yl)-4-(4-chlorophenyl)pyrrolidine-3- | Inhibitor |
| | carboxamide | |
| SRPK1 | 2,3-Di(thiophen-2-yl)benzo[g]quinoxaline-8-carboxylic acid\|Sphinx31\|5-methyl-N-[2-(morpholin- | Inhibitor |
| | 4-yl)-5-(trifluoromethyl)phenyl]furan-2-carboxamide\|N-[2-(4-methylpiperazin-1-yl)-5- | |
| | (trifluoromethyl)phenyl]thiophene-2-carboxamide\|SPHINX scaffold,3 | |
| KS6B1 | 2-(2-chloro-4-iodophenylamino)-N-cyclopropylmethoxy-3,4-difluorobenzamide\|Actb-1003\|(2s)-1- | Inhibitor |
| | {[6-Furan-3-Yl-5-(3-Methyl-2h-Indazol-5-Yl)pyridin-3-Yl]oxy}-3-(1h-Indol-3-Yl)propan-2-Amine\|N- | |
| | [2-(4-amino-1,2,5-oxadiazol-3-yl)-1-ethylimidazo[5,4-d]pyridin-7-yl]methyl]piperidin-4-amine\|3- | |
| | (3-chloro-4-hydroxyphenylamino)-4-(4-nitrophenyl)-1H-pyrrole-2,5-dione\|Bisindolylmaleimide | |
| | IV\|(S)-4-(2-(N-methylisoquinoline-5-sulfonamido)-3-oxo-3-(4-phenylpiperazin-1-yl)propyl)phenyl | |
| | isoquinoline-5-sulfonate\|4,5,6,7-Tetrabromobenzotriazole\|#N/A\|bisindolylmaleimide | |
| | IX\|Indiplon\|KT5720\|2-{[4-(5-Ethylpyrimidin-4-Yl)piperazin-1-Yl]methyl}-5-(Trifluoromethyl)-1h- | |
| | Benzimidazole\|M2698 free base\|3-{3-[5-(5,6-Difluoro-1H-indol-2-yl)-2-methoxy-phenyl]- | |
| | thioureido}-benzoic acid\|Dpt-prostaglandin E1 | |
| AKT2 | 4-dodecyl-N-(1,3,4-thiadiazol-2-yl)benzenesulfonamide\|(2s)-1-{[6-Furan-3-Yl-5-(3-Methyl-2h- | Inhibitor |
| | Indazol-5-Yl)pyridin-3-Yl]oxy}-3-(1h-Indol-3-Yl)propan-2-Amine\|Akt inhibitor VIII | |
| TYRO3 | N-[4-(6,7-dimethoxyquinolin-4-yl)oxy-3-fluorophenyl]-4-ethoxy-1-(4-fluoro-2- | Inhibitor |
| | methylphenyl)pyrazole-3-carboxamide\|N-[4-(6,7-dimethoxyquinolin-4-yl)oxy-3-fluorophenyl]-3-(4- | |
| | fluorophenyl)-2,4-dioxo-1-propan-2-ylpyrimidine-5-carboxamide\|4-[2-(Butylamino)-5-pyridin-2- | |
| | ylpyrimidin-4-yl]amino]butan-1-ol\|3-[2-(Butylamino)-5-pyridin-2-ylpyrimidin-4-yl]amino]propan-1- | |
| | ol\|N-{1-[5-Chloro-8-(3-fluorophenyl)cinnolin-7-yl]ethyl}-9H-purin-6-amine\|2-N-butyl-4-N- | |
| | (piperidin-4-ylmethyl)-5-pyridin-2-ylpyrimidine-2,4-diamine\|4-[(4-{3-cyano-2-[4-(7H-pyrrolo[2,3- | |
| | d]pyrimidin-4-yl)-1H-pyrazol-1-yl]propyl}piperazin-1-yl)carbonyl]-3-fluorobenzonitrile\|4-{3- | |
| | (cyanomethyl)-3-[4-(7H-pyrrolo[2,3-d]pyrimidin-4-yl)-1H-pyrazol-1-yl]azetidin-1-yl}-N-[4-fluoro-2- | |
| | (trifluoromethyl)phenyl]piperidine-1-carboxamide\|{trans-3-(4-{[4-{[(2S)-2-(hydroxymethyl) | |
| | pyrrolidin-1-yl]methyl}-6-(trifluoromethyl)pyridin-2-yl]oxy}piperidin-1-yl)-1-[4-(7H-pyrrolo[2,3- | |
| | d]pyrimidin-4-yl)-1H-pyrazol-1-yl]cyclobutyl}acetonitrile | |
| M3K14 | 4-{3-[2-Amino-5-(2-Methoxyethoxy)pyrimidin-4-Yl]-1h-Indol-5-Yl}-2-Methylbut-3-Yn-2-Ol | Inhibitor |
| KS6A6 | (1S,2S,3R,4R)-3-((5-chloro-2-(((S)-1-methoxy-7-morpholino-6,7,8,9-tetrahydro-5H- | Inhibitor |
| | benzo[7]annulen-2-yl)amino)pyrimidin-4-yl)amino)bicyclo[2.2.1]hept-5-ene-2-carboxamide.\|2- | |
| | ((3,5-difluoro-4-hydroxyphenyl)amino)-8-isopentyl-5,7-dimethyl-7,8-dihydropteridin-6(5H)- | |
| | one\|(R)-1-benzyl-N-(9-methyl-6-oxo-6,7,8,9-tetrahydropyrido[3″,2″:4,5]pyrrolo[1,2-a]pyrazin-2- | |
| | yl)-1H-pyrazole-4-carboxamide\|Synthesis of (R) inverted question markN-(1-(4-aminobenzyl)- | |
| | 1H-pyrazol-4-yl)-9-methyl-6-oxo-6,7,8,9-tetrahydropyrido[3′,2′:4,5]pyrrolo[1,2-a]pyrazine-2- | |
| | carboxamide hydrochloride salt | |
| PK3CG | 2-PyridinecarboxaMide,N-(5-fluoro-2-pyridinyl)-4-[(5-fluoro-3-pyridinyl)oxy]-6-Methyl-\|2-(5- | Inhibitor\| |
| | Bromo-1h-indol-3-yl)-1h-phenanthro[9,10-d]imidazole\|3-benzyl-5-fluorobenzo[d]oxazol-2(3H)- | Modulator |
| | one\|Escitalopram\|(1R,3R)-3-[4-(2-methylnonan-2-yl)phenyl]cyclohexan-1-ol\|1-Ethyl-3-[3-(3,4,5- | |
| | trimethoxyanilino)pyrido[2,3-b]pyrazin-6-yl]thiourea\|Quinoline,6-(1-methyl-1- | |
| | (methylsulfonyl)ethyl)-8-(3-((1E)-2-(3-methyl-1,2,4-oxadiazol-5-yl)-2-(4- | |
| | (methylsulfonyl)phenyl)ethenyl)phenyl)-\|4-(((2S,3R,4S,5S,6R)-3,4,5-Trihydroxy-6- | |
| | (hydroxymethyl)tetrahydro-2H-pyran-2-yl)oxy)benzaldehyde\|4-[7-Bromo-1-[(2,5- | |
| | difluorophenyl)methyl]benzimidazol-5-yl]sulfonyl-5-methylsulfanylthiophene-2- | |
| | carboximidamide\|4-(4-cyano-2-fluorophenyl)-2-morpholino-5-(1H-1,2,4-triazol-5-yl)thiophene-3- | |
| | carbonitrile\|1-(5-Bromopyridin-2-yl)-3-(2-thiophen-2-ylethyl)thiourea\|Ethyl 6-(5- | |
| | (phenylsulfonamido)pyridin-3-yl)imidazo[1,2-a]pyridine-3-carboxylate\|2-(4-ethylpiperazin-1-yl)-N- | |
| | [4-(2-morpholin-4-yl-4-oxochromen-8-yl)dibenzothiophen-1-yl]acetamide\|7-methyl-2-morpholino- | |
| | 9-(1-(phenylamino)ethyl)-4H-pyrido[1,2-a]pyrimidin-4-one\|(5E)-5-[(2,2-Difluoro-1,3-benzodioxol- | |
| | 5-YL)methylene]-1,3-thiazolidine-2,4-dione\|4-(6-((4-(cyclopropylmethyl)piperazin-1-yl)methyl)-2- | |
| | (5-fluoro-1H-indol-4-yl)thieno[3,2-d]pyrimidin-4-yl)morpholine\|N-[(E)-(6-Bromoimidazo[1,2- | |
| | alpyridin-3-yl)methylideneamino]-N,2-dimethyl-5-nitrobenzenesulfonamide\|1-cyclopentyl-3-(1H- | |
| | pyrrolo[2,3-b]pyridin-5-yl)-1H-pyrazolo[3,4-d]pyrimidin-4-amine\|5-(2-Amino-8-fluoro- | |
| | [1,2,4]triazolo[1,5-a]pyridin-6-yl)-N-tert-butylpyridine-3-sulfonamide\|Myricetin\|5-Quinoxalin-6- | |
| | ylmethylene-thiazolidine-2,4-dione\|Nvp-bag956\|PI-103\|2-(4-Morpholinyl)-4H-1-benzopyran-4- | |
| | one\|2-(Morpholin-4-yl)-benzo[h]chromen-4-one\|3-(4-Morpholin-4-ylthieno[3,2-d]pyrimidin-2- | |
| | yl)phenol\|1-(4-(3-ethyl-7-morpholino-3H-[1,2,3]triazolo[4,5-d]pyrimidin-5-yl)phenyl)-3-(4-(4- | |
| | methylpiperazine-1-carbonyl)phenyl)urea\|3-Dimethylaminomethyl-1H-indol-4- | |
| | ol\|Wortmannin[(3aR,6Z,9S,9aR,10R,11aS)-5-hydroxy-9-(methoxymethyl)-9a,11a-dimethyl- | |
| | 1,4,7-trioxo-6-(pyrrolidin-1-ylmethylidene)-2,3,3a,9,10,11-hexahydroindeno[4,5-h]isochromen- | |
| | 10-yl]acetate\|(Z)-5-((4-(pyridin-4-yl)quinolin-6-yl)methylene)thiazolidine-2,4- | |
| | dione\|Hydroxyisoleucine\|2-(4-Morpholinyl)-8-phenyl-4H-1-benzopyran-4- | |
| | one\|DCCCyB\|Omipalisib\|4,4′-(6-(2-(Difluoromethyl)-1H-benzo[d]imidazol-1-yl)-1,3,5-triazine-2,4- | |
| | diyl)dimorpholine\|Perifosine\|5-(9-Isopropyl-8-methyl-2-morpholino-9H-purin-6-yl)pyrimidin-2- | |
| | amine\|Cortisone acetate\|(R)-8-(1-((3,5-difluorophenyl)amino)ethyl)-N,N-dimethyl-2-morpholino- | |
| | 4-oxo-4H-chromene-6-carboxamide\|4-[4-(3-Hydroxyphenyl)phenyl]-2-methylphenol\|5-(7- | |
| | (methylsulfonyl)-2-morpholino-6,7-dihydro-5H-pyrrolo[2,3-d]pyrimidin-4-yl)pyrimidin-2- | |
| | amine\|Fimepinostat\|6,7-Bis(3-hydroxyphenyl)pteridine-2,4-diamine\|18-(p-Iodophenyl)octadecyl | |
| | phosphocholine\|Ceftriaxone\|BGT-226 free base\|Gdc- | |
| | 0084\|Bimiralisib\|Inavolisib\|Eganelisib\|Pictilisib\|Sonolisib\|Rotungenic acid\|Voxtalisib\|N-[3-(2,1,3- | |
| | benzothiadiazol-5-ylamino)quinoxalin-2-yl]-4-methylbenzenesulfonamide\|CID | |
| | 66577114\|Gedatolisib\|Apitolisib\|PF- | |
| | 04691502\|Dactolisib\|Rigosertib\|Buparlisib\|Enzastaurin\|Taselisib\|Duvelisib\|Copanlisib | |
| TAOK1 | 5-(2-aminopyrimidin-4-yl)-2-phenyl-1H-pyrrole-3-carboxamide | Inhibitor |
| MKNK1 | Tomivosertib | Inhibitor |
| MYLK | 1-(3-Hydroxybenzyl)-3-(4-(pyridin-4-yl)thiazol-2-yl)urea\|2-[(n- | Inhibitor |
| | Butoxycarbonylmethyl)sulfinyl]thiazolo[5,4-b]pyridine | |
| DYRK3 | Harmine\|N-[5-[3-cyano-7-(cyclopropylamino)pyrazolo[1,5-a]pyrimidin-5-yl]amino]-2- | Inhibitor |
| | methylphenyl]acetamide\|Dcdpc\|3-(2,7-Dimethoxyacridin-9-ylthio)propan-1-amine | |
| ATR | 3-Amino-6-(4-(methylsulfonyl)phenyl)-N-phenylpyrazine-2-carboxamide\|Diphenyl | Inhibitor |
| | acetamidotrichloroethyl fluoronitrophenyl thioureal(3R)-4-[2-(1H-indol-4-yl)-6-(1- | |
| | methylsulfonylcyclopropyl)pyrimidin-4-yl]-3-methylmorpholine\|Berzosertib\|Elimusertib\|2-amino-6- | |
| | fluoro-N-(5-fluoro-4-(4-(4-(oxetan-3-yl)piperazine-1-carbonyl)piperidin-1-yl)pyridin-3- | |
| | yl)pyrazolo[1,5-a]pyrimidine-3-carboxamide\|9-Mesityl-10-methylacridinium Perchlorate\|Bet-IN- | |
| | 4\|Ceralasertib | |
| ATM | Diphenyl acetamidotrichloroethyl fluoronitrophenyl thiourea\|KU-60019\|7-Fluoro-3-methyl-8-[6-(3- | Inhibitor |
| | piperidin-1-ylpropoxy)pyridin-3-yl]-1-propan-2-ylimidazo[4,5-c]quinolin-2-one\|Aminomalonic | |
| | acid\|2H-Imidazo[4,5-c]quinolin-2-one,8-[6-[3-(dimethylamino)propoxy]-3-pyridinyl]-1,3-dihydro- | |
| | 3-methyl-1-(tetrahydro-2H-pyran-4-yl)- | |
| M3K5 | N-(5-(cyclopropylamino)-7-(trifluoromethyl)-[1,2,4]triazolo[1,5-a]pyridin-2-yl)nicotinamide\|4-Tert- | Inhibitor |
| | Butyl-N-[6-(1h-Imidazol-1-Yl)imidazo[1,2-A]pyridin-2-Yl]benzamide\|N-(3,3-Dimethyl-butyl)-4- | |
| | indol-1-yl-benzamide\|Selonsertib | |
| AURKC | 4-amino-6-(4-octylphenyl)hexanoic Acid\|Mercaptopurine\|N-Propyl-2-phenylnorapomorphine | Inhibitor\| |
| | hydrochloride\|N-(5-methyl-1H-pyrazol-3-yl)-2-phenylquinazolin-4-amine\|H-1152 Glycyl | Modulator |
| | Dihydrochloride\|Aurora Kinase Inhibitor II\|Aurora Kinase Inhibitor III\|3-((R)-1-((2S,6R)-2,6- | |
| | dimethylmorpholino)ethyl)-N-(6-methyl-3-(1H-pyrazol-4-yl)imidazo[1,2-a]pyrazin-8-yl)isothiazol- | |
| | 5-amine\|N-(3-fluorophenyl)-2-(2-(7-(3-(4-(hydroxymethyl)piperidin-1-yl)propoxy)-6- | |
| | methoxyquinazolin-4-ylamino)thiazol-5-yl)acetamide\|Indirubin-3-methoxime\|Indirubin-3′- | |
| | monoxime\|6-Bromoindirubin-3′-oxime\|Indirubin-3-acetoxime\|#N/A\|7-Fluoroindirubin-3- | |
| | acetoxime\|7-Bromoindirubin-3-oxime\|7-Bromoindirubin-3-acetoxime\|(3z)-N,N-Dimethyl-2-Oxo-3- | |
| | (4,5,6,7-Tetrahydro-1h-Indol-2-Ylmethylidene)-2,3-Dihydro-1h-Indole-5-Sulfonamide\|Aurora B | |
| | inhibitor 1\|Sns-314\|Sulfadiazine\|3-(4-(4-(2-(3-((dimethylamino)methyl)phenyl)-1H-pyrrolo[2,3- | |
| | b]pyridin-4-yl)-1-ethyl-1H-pyrazol-3-yl)phenyl)-1,1-dimethylurea\|Fluorometholone\|N-(4-(3-(2- | |
| | aminopyrimidin-4-yl)pyridin-2-yloxy)phenyl)-4-(4-methylthiophen-2-yl)phthalazin-1- | |
| | amine\|lorasertib\|Danusertib | |
| M3K20 | 3-[6-(2-pyridin-2-yl-5,6-dihydro-4H-pyrrolo[2,1-e]pyrazol-3-yl)benzimidazol-1-yl]propan-1-ol | Inhibitor |
| GUC2C | #N/A\|N-[(1S,2S)-2-[(6-Fluoro-1,3-benzothiazol-2-yl)amino]cyclopentyl]-2,6-dimethoxy-N- | Inhibitor\| |
| | methylbenzamide\|2-(4-Isobutylcyclohexyl)-2-oxoethyl benzenesulfonate\|Linaclotide\|Linaclotide | Modulator\| |
| | acetate\|Plecanatide | Agonist\| |
| | | Activator |
| CDK19 | SEL120-34A HCl | Inhibitor |
| DCK | H-Glu-Pro-Gln-Tyr(PO3H2)-Glu-Glu-Ile-Pro-Ile-Tyr-Leu-OH | Modulator |
| MYLK2 | 1-(5-(4-amino-7-methyl-7H-pyrrolo[2,3-d]pyrimidin-5-yl)indolin-1-yl)-2-(3- | Inhibitor |
| | (trifluoromethyl)phenyl)ethanone | |
| CLK4 | 5-(benzo[d][1,3]dioxol-5-yl)-N-(3,5-dichlorobenzyl)pyrimidin-4-amine\|4-(1,3-benzodioxol-5-yl)-N- | Inhibitor |
| | [(3,5-dichlorophenyl)methyl]pyrimidin-2-amine | |
| PAK4 | N-((1S)-2-(Dimethylamino)-1-phenylethyl)-6,6-dimethyl-3-((2-methylthieno(3,2-d)pyrimidin-4- | Inhibitor |
| | yl)amino)-4,6-dihydropyrrolo(3,4-C)pyrazole-5(1H)-carboxamide\|Pak4-IN-1 | |
| TIE1 | N-[(3R)-8-[5-chloro-4-[2-fluoro-6-(methylcarbamoyl)anilino]pyrimidin-2-yl]amino]-5,5-dimethyl-2- | Inhibitor |
| | oxo-3,4-dihydro-1H-1-benzazepin-3-yl]pyrrolidine-1-carboxamide | |
| TOPK | (R)-9-(4-(1-(dimethylamino)propan-2-yl)phenyl)-8-hydroxy-6-methylthieno[2,3-c]quinolin-4(5H)- | Inhibitor |
| | one\|N-(12-cyanoindolizino[2,3-b]quinoxalin-2-yl)thiophene-2-carboxamide | |
| MKNK2 | Tomivosertib | Inhibitor |
| RIPK1 | 1-[3-[5-(3-aminophenyl)-1H-pyrrolo[2,3-b]pyridin-3-yl]phenyl]-3-[2-fluoro-5- | Inhibitor |
| | (trifluoromethyl)phenyl]urea\|Gallium citrate\|CID 134203923\|N-benzyl-N-hydroxy-2,2- | |
| | dimethylbutanamide\|rel-(S)-N-((S)-7-Methoxy-1-methyl-2-oxo-2,3,4,5-tetrahydro-1H-pyrido[3,4- | |
| | b]azepin-3-yl)-5-phenyl-6,7-dihydro-5H-pyrrolo[1,2-b][1,2,4]triazole-2-carboxamide\|Fevipiprant\|2- | |
| | (Methylthio)thiazole\|Fulacimstat\|5-benzyl-N-[(3S)-7,9-difluoro-2-oxo-1,3,4,5-tetrahydro-1- | |
| | benzazepin-3-yl]-1H-1,2,4-triazole-3-carboxamide\|(S)-5-benzyl-N-(5-methyl-4-oxo-2,3,4,5- | |
| | tetrahydrobenzo[b][1,4]oxazepin-3-yl)-1H-1,2,4-triazole-3-carboxamide\|3,4-Dibutoxy-3- | |
| | cyclobutene-1,2-dione\|5-(1H-indol-3-ylmethyl)-3-methylimidazolidine-2,4-dione\|4-Hydroxy-3-(4- | |
| | hydroxy-3-nitrocinnamoyl)-6-methyl-2H-pyran-2-one\|1-[3-(4-Fluorophenyl)-8-methyl-3,3a,4,5- | |
| | tetrahydrobenzo[g]indazol-2-yl]ethanone | |
| KC1E | 3-[(2,4,6-Trimethoxy-phenyl)-methylene]-indolin-2-one\|3-[(3-Chlorophenoxy)methyl]-1- | Inhibitor |
| | (Tetrahydro-2h-Pyran-4-Yl)-1h-Pyrazolo[3,4-D]pyrimidin-4-Amine\|PF-670462\|Umbralisib | |
| BMX | Blk-IN-1\|2-methyl-N-[2-({3-[2-(prop-2-enamido)acetamido]phenyl}amino)pyrimidin-5-yl]-5-[3- | Inhibitor |
| | (trifluoromethyl)benzamido]benzamide | |

TABLE 11
Cas enzyme sequences

| Cas | SEQ ID | Amino Acid Sequence |
|---|---|---|
| protein | NO | |
| dCas9 | 12950 | MDKKYSIGLAIGTNSVGWAVITDEYKVPSKKFKVLGNTDRHSIKKNLIGALLFDSGETAEATRLKRTARR |
| | | RYTRRKNRICYLQEIFSNEMAKVDDSFFHRLEESFLVEEDKKHERHPIFGNIVDEVAYHEKYPTIYHLRK |
| | | KLVDSTDKADLRLIYLALAHMIKFRGHFLIEGDLNPDNSDVDKLFIQLVQTYNQLFEENPINASGVDAKAI |
| | | LSARLSKSRRLENLIAQLPGEKKNGLFGNLIALSLGLTPNFKSNFDLAEDAKLQLSKDTYDDDLDNLLA |
| | | QIGDQYADLFLAAKNLSDAILLSDILRVNTEITKAPLSASMIKRYDEHHQDLTLLKALVRQQLPEKYKEIF |
| | | FDQSKNGYAGYIDGGASQEEFYKFIKPILEKMDGTEELLVKLNREDLLRKQRTFDNGSIPHQIHLGELH |
| | | AILRRQEDFYPFLKDNREKIEKILTFRIPYYVGPLARGNSRFAWMTRKSEETITPWNFEEVVDKGASAQ |
| | | SFIERMTNFDKNLPNEKVLPKHSLLYEYFTVYNELTKVKYVTEGMRKPAFLSGEQKKAIVDLLFKTNRK |
| | | VTVKQLKEDYFKKIECFDSVEISGVEDRFNASLGTYHDLLKIIKDKDFLDNEENEDILEDIVLTLTLFEDRE |
| | | MIEERLKTYAHLFDDKVMKQLKRRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDELVKVMGRHKPENIVIEMARENQ |
| | | TTQKGQKNSRERMKRIEEGIKELGSQILKEHPVENTQLQNEKLYLYYLQNGRDMYVDQELDINRLSDY |
| | | DVDAIVPQSFLKDDSIDNKVLTRSDKNRGKSDNVPSEEVVKKMKNYWRQLLNAKLITQRKFDNLTKAE |
| | | RGGLSELDKAGFIKRQLVETRQITKHVAQILDSRMNTKYDENDKLIREVKVITLKSKLVSDFRKDFQFYK |
| | | VREINNYHHAHDAYLNAVVGTALIKKYPKLESEFVYGDYKVYDVRKMIAKSEQEIGKATAKYFFYSNIM |
| | | NFFKTEITLANGEIRKRPLIETNGETGEIVWDKGRDFATVRKVLSMPQVNIVKKTEVQTGGFSKESILPK |
| | | RNSDKLIARKKDWDPKKYGGFDSPTVAYSVLVVAKVEKGKSKKLKSVKELLGITIMERSSFEKNPIDFL |
| | | EAKGYKEVKKDLIIKLPKYSLFELENGRKRMLASAGELQKGNELALPSKYVNFLYLASHYEKLKGSPED |
| | | NEQKQLFVEQHKHYLDEIIEQISEFSKRVILADANLDKVLSAYNKHRDKPIREQAENIIHLFTLTNLGAPA |
| | | AFKYFDTTIDRKRYTSTKEVLDATLIHQSITGLYETRIDLSQLGGD |
| dCas12a | 12951 | MTQFEGFTNLYQVSKTLRFELIPQGKTLKHIQEQGFIEEDKARNDHYKELKPIIDRIYKTYADQCLQLVQ |
| | | LDWENLSAAIDSYRKEKTEETRNALIEEQATYRNAIHDYFIGRTDNLTDAINKRHAEIYKGLFKAELFNG |
| | | KVLKQLGTVTTTEHENALLRSFDKFTTYFSGFYENRKNVFSAEDISTAIPHRIVQDNFPKFKENCHIFTR |
| | | LITAVPSLREHFENVKKAIGIFVSTSIEEVFSFPFYNQLLTQTQIDLYNQLLGGISREAGTEKIKGLNEVLN |
| | | LAIQKNDETAHIIASLPHRFIPLFKQILSDRNTLSFILEEFKSDEEVIQSFCKYKTLLRNENVLETAEALFNE |
| | | LNSIDLTHIFISHKKLETISSALCDHWDTLRNALYERRISELTGKITKSAKEKVQRSLKHEDINLQEIISAAG |
| | | KELSEAFKQKTSEILSHAHAALDQPLPTTLKKQEEKEILKSQLDSLLGLYHLLDWFAVDESNEVDPEFS |
| | | ARLTGIKLEMEPSLSFYNKARNYATKKPYSVEKFKLNFQMPTLASGWDVNKEKNNGAILFVKNGLYYL |
| | | GIMPKQKGRYKALSFEPTEKTSEGFDKMYYDYFPDAAKMIPKCSTQLKAVTAHFQTHTTPILLSNNFIE |
| | | PLEITKEIYDLNNPEKEPKKFQTAYAKKTGDQKGYREALCKWIDFTRDFLSKYTKTTSIDLSSLRPSSQY |
| | | KDLGEYYAELNPLLYHISFQRIAEKEIMDAVETGKLYLFQIYNKDFAKGHHGKPNLHTLYWTGLFSPEN |
| | | LAKTSIKLNGQAELFYRPKSRMKRMAHRLGEKMLNKKLKDQKTPIPDTLYQELYDYVNHRLSHDLSDE |
| | | ARALLPNVITKEVSHEIIKDRRFTSDKFFFHVPITLNYQAANSPSKFNQRVNAYLKEHPETPIIGIDRGER |
| | | NLIYITVIDSTGKILEQRSLNTIQQFDYQKKLDNREKERVAARQAWSVVGTIKDLKQGYLSQVIHEIVDLM |
| | | IHYQAVVVLANLNFGFKSKRTGIAEKAVYQQFEKMLIDKLNCLVLKDYPAEKVGGVLNPYQLTDQFTSF |
| | | AKMGTQSGFLFYVPAPYTSKIDPLTGFVDPFVWKTIKNHESRKHFLEGFDFLHYDVKTGDFILHFKMNR |
| | | NLSFQRGLPGFMPAWDIVFEKNETQFDAKGTPFIAGKRIVPVIENHRFTGRYRDLYPANELIALLEEKGI |
| | | VFRDGSNILPKLLENDDSHAIDTMVALIRSVLQMRNSNAATGEDYINSPVRDLNGVCFDSRFQNPEWP |
| | | MDADANGAYHIALKGQLLLNHLKESKDLKLQNGISNQDWLAYIQELRN |
| dCas12f1 | 12952 | MAKNTITKTLKLRIVRPYNSAEVEKIVADEKNNREKIALEKNKDKVKEACSKHLKVAAYCTTQVERNACL |
| | | FCKARKLDDKFYQKLRGQFPDAVFWQEISEIFRQLQKQAAEIYNQSLIELYYEIFIKGKGIANASSVEHY |
| | | LSDVCYTRAAELFKNAAIASGLRSKIKSNFRLKELKNMKSGLPTTKSDNFPIPLVKQKGGQYTGFEISN |
| | | HNSDFIIKIPFGRWQVKKEIDKYRPWEKFDFEQVQKSPKPISLLLSTQRRKRNKGWSKDEGTEAEIKKV |
| | | MNGDYQTSYIEVKRGSKICEKSAWMLNLSIDVPKIDKGVDPSIIGGIAVGVKSPLVCAINNAFSRYSISD |
| | | NDLFHFNKKMFARRRILLKKNRHKRAGHGAKNKLKPITILTEKSERFRKKLIERWACEIADFFIKNKVGT |
| | | VQMENLESMKRKEDSYFNIRLRGFWPYAEMQNKIEFKLKQYGIEIRKVAPNNTSKTCSKCGHLNNYFN |
| | | FEYRKKNKFPHFKCEKCNFKENAAYNAALNISNPKLKSTKEEP |
| dCasMINI | 12953 | MAKNTITKTLKLRIVRPYNSAEVEKIVADEKNNREKIALEKNKDKVKEACSKHLKVAAYCTTQVERNACL |
| | | FCKARKLDDKFYQKLRGQFPDAVFWQEISEIFRQLQKQAAEIYNQSLIELYYEIFIKGKGIANASSVEHY |
| | | LSRVCYRRAAELFKNAAIASGLRSKIKSNFRLKELKNMKSGLPTTKSDNFPIPLVKQKGGQYTGFEISN |
| | | HNSDFIIKIPFGRWQVKKEIDKYRPWEKFDFEQVQKSPKPISLLLSTQRRKRNKGWSKDEGTEAEIKKV |
| | | MNGDYQTSYIEVKRGSKICEKSAWMLNLSIDVPKIDKGVDPSIIGGIAVGVRSPLVCAINNAFSRYSISD |
| | | NDLFHFNKKMFARRRILLKKNRHKRAGHGAKNKLKPITILTEKSERFRKKLIERWACEIADFFIKNKVGT |
| | | VQMENLESMKRKEDSYFNIRLRGFWPYAEMQNKIEFKLKQYGIEIRKVAPNNTSKTCSKCGHLNNYFN |
| | | FEYRKKNKFPHFKCEKCNFKENAAYNAALNISNPKLKSTKERP |

TABLE 12
Confusion matrix table for sorafenib response prediction

| | CRISPRi− | CRISPRi− | | | | |
|---|---|---|---|---|---|---|
| | modulated | modulated | | | | |
| | predicted | predicted non- | FLT3 | FLT3 | FLT3/KIT | FLT3/KIT |
| | responders | responders | Mut+ | Mut− | Mut+ | Mut− |
| Observed | 11 | 3 | 9 | 5 | 10 | 4 |
| responders | | | | | | |
| Observed non- | 1 | 7 | 4 | 4 | 5 | 3 |
| responders | | | | | | |

TABLE 13
Linker, NLS, and T2A peptide sequences

| | SEQ ID | |
|---|---|---|
| Vector Element | NO: | Amino Acid Sequence |
| Linker | 12954 | YPTFLYKVVGGSGGSA |
| SV40 NLS | 12955 | PKKKRKV |
| SV40 NLS | 12956 | PKKKRKVD |
| SV40 NLS | 12957 | PKKKRKVG |
| Triple SV40 NLS | 12958 | PKKKRKVDPKKKRKVDPKKKRKVG |
| nucleoplasmin | 12959 | KRPAATKKAGQAKKKK |
| NLS | | |
| T2A peptide | 12960 | EGRGSLLTCGDVEENPGP |

TABLE 14
Vector nucleotide sequences

| | | | Seq ID | |
|---|---|---|---|---|
| Name | Source | mol_type | NO: | Nucleotide sequence |
| RSV promoter | Synthetic | other DNA | 12961 | tgtagtcttatgcaatactcttgtagtcttgcaacatggtaacgatgagttagcaac |
| | construct | | | atgccttacaaggagagaaaaagcaccgtgcatgccgattggtggaagtaagg |
| | | | | tggtacgatcgtgccttattaggaaggcaacagacgggtctgacatggattgga |
| | | | | cgaaccactgaattgccgcattgcagagatattgtatttaagtgcctagctcgata |
| | | | | cataaac |
| Truncated 5′ | Synthetic | other DNA | 12962 | tgtagtcttatgcaatactcttgtagtcttgcaacatggtaacgatgagttagcaac |
| LTR | construct | | | atgccttacaaggagagaaaaagcaccgtgcatgccgattggtggaagtaagg |
| | | | | tggtacgatcgtgccttattaggaaggcaacagacgggtctgacatggattgga |
| | | | | cgaaccactgaattgccgcattgcagagatattgtatttaagtgcctagctcgata |
| | | | | cataaac |
| Human U6 | Homo | Genomic | 12963 | gagggcctatttcccatgattccttcatatttgcatatacgatacaaggctgttaga |
| promoter - | sapiens | DNA | | gagataattggaattaatttgactgtaaacacaaagatattagtacaaaatacgtg |
| full length | | | | acgtagaaagtaataatttcttgggtagtttgcagttttaaaattatgttttaaaatgg |
| | | | | actatcatatgcttaccgtaacttgaaagtatttcgatttcttggctttatatatcttgt |
| | | | | ggaaaggacgaaacaccg |
| Human U6 | Homo | Genomic | 12964 | gagggcctatttcccatgattccttcatatttgcatatacgatagottaccgtaactt |
| promoter - | sapiens | DNA | | gaaagtatttcgatttcttggctttatatatcttgtggaaaggacgaaacaccg |
| minimum | | | | |
| minimal | Synthetic | other DNA | 12965 | atatttagcatgtcgctatgtgttctgggaaacttgacctaagtgtaaagttgagatt |
| compound | construct | | | tccttcaggtttatatagttctgtatgagaccactctttccc |
| pol-III | | | | |
| promoter M11 | | | | |
| tracrRNA-1 | Synthetic | other DNA | 12966 | gtttgagagctaagcagaaagctgcatagcaagttcaaataaggctagtccgta |
| | construct | | | cacaacttgaaaaagtggcagccgagtcggctgc |
| tracrRNA-1 | Synthetic | other DNA | 12967 | gtttgagagctaagcagaannnnnnagctgcatagcaagttcaaataaggcta |
| iBAR | construct | | | gtccgtacacaacttgaaaaagtggcagccgagtcggctgc |
| tracrRNA-2 | Synthetic | other DNA | 12968 | gtttcagagctaagcacaagagtgcatagcaagttgaaataaggctagtccgttt |
| | construct | | | acaacttggacttcggtccaagtggcacccgagtcgggtgc |
| tracrRNA-3 | Synthetic | other DNA | 12969 | gtttaagagctatgctggaaacagcatagcaagtttaaataaggctagtccgttat |
| | construct | | | caacttgaaaaagtggcaccgagtcggtgc |
| UbiC | Homo | Genomic | 12970 | ggcctccgcgccgggttttggcgcctcccgcgggcgcccccctcctcacggc |
| promoter | sapiens | DNA | | gagcgctgccacgtcagacgaagggcgcagcgagcgtcctgatccttccgcc |
| | | | | cggacgctcaggacagcggcccgctgctcataagactcggccttagaacccc |
| | | | | agtatcagcagaaggacattttaggacgggacttgggtgactctagggcactgg |
| | | | | ttttctttccagagagcggaacaggcgaggaaaagtagtcccttctcggcgattc |
| | | | | tgcggagggatctccgtggggcggtgaacgccgatgattatataaggacgcgc |
| | | | | cgggtgtggcacagctagttccgtcgcagccgggatttgggtcgcggttcttgtt |
| | | | | tgtggatcgctgtgatcgtcacttggt |
| T2A | Synthetic | other DNA | 12971 | gagggcagaggaagccttctaacatgcggtgacgtggaggagaatcccggcc |
| | construct | | | ct |
| T2A-with | Synthetic | other DNA | 12972 | ggatccgagggcagaggaagccttctaacatgcggtgacgtggaggagaatc |
| linkers | construct | | | ccggccctcccggg |
| Thy1 marker | Synthetic | other DNA | 12973 | atgaacccagccatcagcgtcgctctcctgctctcagtcttgcaggtgtcccgag |
| sequence | construct | | | ggcagaaggtgaccagcctgacagcctgcctggtgaaccaaaaccttcgcctg |
| | | | | gactgccgccatgagaataacaccaaggataactccatccagcatgagttcag |
| | | | | cctgaccegagagaagaggaagcacgtgctctcaggcaccctegggataccc |
| | | | | gagcacacgtaccgctcccgegtcaccctctccaaccagccctatatcaaggtc |
| | | | | cttaccctagccaacttcaccaccaaggatgagggcgactacttttgtgagcttc |
| | | | | gagtctcgggcgcgaatcccatgagctccaataaaagtatcagtgtgtatagag |
| | | | | acaaactggtcaagtgtggcggcataagcctgctggttcagaacacatcctgga |
| | | | | tgctgctgctgctgctttccctctccctcctccaagccctggacttcatttctctgtg |
| | | | | a |
| 3′ LTR | Synthetic | other DNA | 12974 | tggaagggctaattcactcccaacgaagataagatctgctttttgcttgtactggg |
| | construct | | | tctctctggttagaccagatctgagcctgggagctctctggctaactagggaacc |
| | | | | cactgcttaagcctcaataaagcttgccttgagtgcttcaagtagtgtgtgcccgt |
| | | | | ctgttgtgtgactctggtaactagagatccctcagacccttttagtcagtgtggaa |
| | | | | aatctctagca |
| SV40 | Synthetic | other DNA | 12975 | aacttgtttattgcagcttataatggttacaaataaagcaatagcatcacaaatttca |
| polyadenyla- | construct | | | caaataaagcatttttttcactgcattctagttgtggtttgtccaaactcatcaatgta |
| tion | | | | tctta |
| signal | | | | |
| Ori sequence | Synthetic | other DNA | 12976 | tttccataggctccgcccccctgacgagcatcacaaaaatcgacgctcaagtca |
| | construct | | | gaggtggcgaaacccgacaggactataaagataccaggcgtttccccctggaa |
| | | | | gctccctcgtgcgctctcctgttccgaccctgccgcttaccggatacctgtccgc |
| | | | | ctttctcccttcgggaagcgtggcgctttctcatagctcacgctgtaggtatctca |
| | | | | gttcggtgtaggtcgttcgctccaagctgggctgtgtgcacgaaccccccgttca |
| | | | | gcccgaccgctgcgccttatccggtaactatcgtcttgagtccaacccggtaag |
| | | | | acacgacttatcgccactggcagcagccactggtaacaggattagcagagcga |
| | | | | ggtatgtaggggtgctacagagttcttgaagtggtggcctaactacggctaca |
| | | | | ctagaagaacagtatttggtatctgcgctctgctgaagccagttaccttcggaaa |
| | | | | aagagttggtagctcttgatccggcaaacaaaccaccgctggtagcggtggtttt |
| | | | | tttgtttgcaagcagcagattacgcgcagaaaaaaaggatctcaa |
| AmpR | Synthetic | other DNA | 12977 | atgagtattcaacatttccgtgtcgcccttattcccttttttgcggcattttgccttcct |
| | construct | | | gtttttgctcacccagaaacgctggtgaaagtaaaagatgctgaagatcagttgg |
| | | | | gtgcacgagtgggttacatcgaactggatctcaacageggtaagatccttgaga |
| | | | | gttttcgccccgaagaacgttttccaatgatgagcacttttaaagttctgctatgtg |
| | | | | gcgcggtattatcccgtattgacgccgggcaagagcaactcggtcgccgcata |
| | | | | cactattctcagaatgacttggttgagtactcaccagtcacagaaaagcatcttac |
| | | | | ggatggcatgacagtaagagaattatgcagtgctgccataaccatgagtgataa |
| | | | | cactgcggccaacttacttctgacaacgatcggaggaccgaaggagctaaccg |
| | | | | cttttttgcacaacatgggggatcatgtaactcgccttgatcgttgggaaccgga |
| | | | | gctgaatgaagccataccaaacgacgagcgtgacaccacgatgcctgtagca |
| | | | | atggcaacaacgttgcgcaaactattaactggcgaactacttactctagcttccc |
| | | | | ggcaacaattaatagactggatggaggcggataaagttgcaggaccacttctgc |
| | | | | gctcggcccttccggctggctggtttattgctgataaatctggagccggtgagcg |
| | | | | tgggtctcgcggtatcattgcagcactggggccagatggtaagccctcccgtat |
| | | | | cgtagttatctacacgacggggagtcaggcaactatggatgaacgaaatagac |
| | | | | agatcgctgagataggtgcctcactgattaagcattggtaa |
| AmpR | Synthetic | other DNA | 12978 | cgcggaacccctatttgtttatttttctaaatacattcaaatatgtatccgctcatgag |
| promoter | construct | | | acaataaccctgataaatgcttcaataatattgaaaaaggaagagt |
| i7 index | Synthetic | other DNA | 12979 | gatcggaagagcacacgtctgaactccagtcacnnnnnnnnnngctagcaa |
| binding | construct | | | ggtccctatagtgagtcgtattagaattggcgcgccaa |
| site - | | | | |
| Random | | | | |
| Sequence | | | | |
| Label | | | | |
| central | Synthetic | other DNA | 12980 | tttaaaagaaaaggggggattggggggtacagtgcaggggaaagaatagtag |
| polypurine | construct | | | acataatagcaacagacatacaaactaaagaattacaaaaacaaattacaaaaa |
| tract and | | | | ttcaaaattt |
| central | | | | |
| termination | | | | |
| sequence of | | | | |
| HIV-1 | | | | |
| 3X-SV40 NLS | Synthetic | other DNA | 12981 | ccaaaaaagaagagaaaggtagatcctaagaaaaagcgtaaggttgatccaaa |
| nucleotide | construct | | | gaagaaaaggaaagtggg |

What is claimed is:

1. A method of treating cancer in a subject in need thereof, the method comprising administering to the subject a therapeutic molecule selected from a compilation of therapeutic molecules; wherein the therapeutic molecule has been selected by a method comprising: modifying cancer cells from the subject by knocking down the function of a plurality of genes without targeted DNA cleavage of one or more genomic loci to generate modified cancer cells, each gene in the plurality of genes encoding for a protein target of a therapeutic molecule in the compilation of therapeutic molecules, whereby the therapeutic molecule has been selected if knocking down the function of the gene that encodes for the protein target of the selected therapeutic molecule impairs cancer cell viability or proliferation rate during in vitro propagation.

2. The method of claim 1, wherein modifying cancer cells from the subject by knocking down the function of the plurality of genes comprises knocking down gene transcription of the plurality of genes.

3. The method of claim 2, wherein knocking down gene transcription of the plurality of genes comprises (i) reducing mRNA expression of the plurality of genes to below a threshold level, and/or (ii) altering a phenotype of the modified cancer cells.

4. The method of claim 2, wherein modifying cancer cells comprises perturbing the function of the plurality of genes without induction of a double-strand break (DSB) response in a majority of the modified cancer cells, wherein a majority comprises greater than 50% of the modified cancer cells.

5. The method of claim 3, wherein knocking down the function of the plurality of genes comprises using CRISPR interference (CRISPRi).

6. The method of claim 5, wherein using CRISPRi comprises using a catalytically inactive form of a CAS endonuclease.

7. The method of claim 6, wherein the catalytically inactive form of CAS endonuclease comprises dCas9 dCas12a, dCas12e, dCas12f, dCas12f1, dCasMINI, dCas12j2, or dCas12j3.

8. The method of claim 6, wherein the catalytically inactive form of CAS endonuclease is fused to an epigenetic modulator.

9. The method of claim 8, wherein the epigenetic modulator comprises a KRAB (also referred to as KOX), SID, MBD2, MBD3, HP1a, DNMT family (including DNMT1, DNMT3A, DNMT3B, DNMT3L, DNMT2A), Sin3a, Rb, MeCP2, ROM2, AtHD2A, LSD1, SUV39H1, G9a (EHMT2), ZFP1, ZFP14, ZFP82, ZIM2, ZIM3, ZNF18, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF419, ZNF140, ZNF175, ZNF214, ZNF184, ZNF8, ZNF60, ZNF595, ZNF596, ZNF10, ZNF37A, ZNF394, ZNF610, ZNF273, ZNF34, ZNF250, ZNF98, ZNF675, ZNF213, ZFP28-2, ZNF224, ZNF257, GLP (EHMT2), or a variant or combination thereof.

10. The method of claim 9, comprising the KRAB, wherein the KRAB comprises a KRAB, or variant thereof, selected from Table 1 or Table 2.

11. The method of claim 9, comprising the KRAB, wherein the KRAB comprises a ZNF10 KRAB.

12. The method of any one of claims 8-11, wherein the catalytically inactive form of CAS endonuclease is fused to the epigenetic modulator via a linker.

13. The method of claim 12, wherein the linker has about one to about 100 amino acids, optionally a flexible linker, and further optionally the linker is about 16 amino acids.

14. The method of any one of claims 6-13, wherein the catalytically inactive form of the CAS endonuclease comprises a nuclear localization sequence (NLS) positioned amino-terminal (N-terminal) to a RuvC-1 domain of the catalytically inactive form of the CAS endonuclease.

15. The method of claim 14, wherein the NLS positioned N-terminal to the RuvC-1 domain comprises a triple SV40 NLS.

16. The method of any one of claims 6-15, wherein the catalytically inactive form of the CAS endonuclease comprises an NLS positioned carboxy-terminal (C-terminal) to a RuvC-III domain of the catalytically inactive form of the CAS endonuclease.

17. The method of claim 16, wherein the NLS positioned C-terminal to the RuvC-III domain comprises a single nucleoplasmin NLS.

18. The method of any one of claims 6-17, wherein the catalytically inactive form of the CAS endonuclease comprises a peptide-linked downstream reporter C-terminal to a RuvC-III domain of the catalytically inactive form of the CAS endonuclease.

19. The method of claim 18, wherein the peptide-linked downstream reporter C-terminal to the RuvC-III domain comprises a self-cleaving T2A-peptide linked downstream reporter.

20. The method of claim 19, wherein the self-cleaving T2A-peptide linked downstream reporter is linked to the catalytically inactive form of CAS endonuclease C-terminal relative to the NLS positioned C-terminal to the RuvC-III domain.

21. The method of claim 19 or 20, wherein the self-cleaving T2A-peptide linked downstream reporter comprises Thy1.

22. The method of any one of claims 8-21, wherein the epigenetic modulator represses transcription of each of the plurality of genes.

23. The method of any one of claims 8-22, wherein the epigenetic modulator inhibits RNA polymerase binding to a promoter sequence near a transcription start site of each of the plurality of genes.

24. The method of any one of claims 8-23, wherein the epigenetic modulator stalls RNA polymerase during mRNA transcription of each of plurality of genes.

25. The method of any one of claims 8-24, wherein the epigenetic modulator inhibits DNA helicase function near a region of genomic DNA for each of the plurality of genes.

26. The method of any one of claims 5-25, wherein using CRISPRi comprises using PAM anchored gene interference without creating double-strand breaks in DNA for each of the plurality of genes.

27. The method of any one of claims 5-26, wherein using CRISPRi comprises using a library of gene modulatory reagents to generate the modified cancer cells.

28. The method of claim 27, wherein the library of gene modulatory reagents comprises a nucleic acid sequence at least 80% identical to at least about 15 contiguous nucleotides of a target gene of Table 3, Table 4, Table 9A, Table 9B, Table 9C, Table 9D, Table 10A, Table 10B, or Table 10C.

29. The method of claim 27 or 28, wherein one or more of the library of gene modulatory reagents comprise a guide RNA (gRNA) sequence comprising complementarity to at least a portion of the gene that encodes a protein target of a therapeutic molecule in the compilation of therapeutic molecules.

30. The method of claim 29, wherein the gRNA sequence further comprises a barcode sequence.

31. The method of claim 30, wherein the barcode sequence comprises an iBAR barcode sequence incorporated into a first stemloop of the gRNA sequence.

32. The method of any one of claims 27-31, wherein the library comprises a plurality of random sequence labels (RSLs) incorporated during library amplification.

33. The method of claim 32, wherein the plurality of random sequence labels comprises a plurality of random sequence barcodes of about 1 to about 15 nucleotides each.

34. The method of any one of claims 29-33, wherein the complementarity to at least a portion of the gene comprises complementarity to the sense strand of each of the plurality of genes.

35. The method of any one of claims 29-34, wherein the complementarity to at least a portion of the gene comprises complementarity to the antisense strand of each of the plurality of genes.

36. The method of any one of claims 29-35, wherein the at least a portion of the gene is downstream of a transcriptional start site for each of the plurality of genes.

37. The method of any one of claims 1-36, wherein a majority of the modified cancer cells do not exhibit a mild non-specific fitness defect.

38. The method of any one of claims 1-4, wherein the knocking down the function of the plurality of genes comprises using Transcription activator-like effector (TALE) repression.

39. The method of claim 38, wherein using TALE repression comprises using an engineered TALE binding protein fused to an epigenetic modulator.

40. The method of claim 39, wherein the epigenetic modulator comprises an epigenetic modulator domain, or variant thereof, which is listed in Table 1 or Table 2.

41. The method of any one of claims 1-4, wherein knocking down the function of the plurality of genes comprises using a catalytically inactive zinc finger nuclease.

42. The method of any one of claims 1-41, wherein modifying cancer cells comprises post-transcriptional knocking down of gene mRNA function.

43. The method of claim 42, wherein post-transcriptional knocking down of gene mRNA function comprises using RNA interference (RNAi).

44. The method of claim 43, wherein using RNAi comprises introducing a plurality of double-stranded RNA (dsRNA) molecules comprising RNA sequence complimentary to mRNA sequences of the plurality of genes into the cancer cells to generate the modified cancer cells.

45. The method of claim 43 or 44, wherein using RNAi comprises introducing a plurality of small hairpin RNA (shRNA) molecules comprising RNA sequence complimentary to mRNA sequences of the plurality of genes into the cancer cells to generate the modified cancer cells.

46. The method of any one of claims 1-45, wherein components used for knocking down the function of the plurality of genes are delivered by a viral vector.

47. The method of claim 46, wherein the viral vector comprises a retroviral vector, an adenoviral vector, an adeno-associated viral vector, or a herpes simplex viral vector.

48. The method of claim 47, wherein the retroviral vector comprises a lentiviral vector.

49. The method of claim 48, wherein the lentiviral vector comprises a pseudotyped lentiviral vector.

50. The method of claim 49, wherein the pseudotyped lentiviral vector comprises a plurality of envelope glycoproteins, wherein the plurality of envelope glycoproteins are derived from Morbillivirus, Sendai virus (SeV), Nipah virus (NiV), Newcastle disease virus (NDV), or Baboon endogenous virus (BaEV), or a combination thereof; and/or wherein the plurality of envelope glycoproteins are attached to an antibody or antibody fragment thereof, including, but not limited to, a single-chain antibody fragment, nanobody, or darpin.

51. The method of any one of claims 1-50, wherein modifying cancer cells comprises depleting a target protein level during in vitro propagation of the modified cancer cells by at least 10%, 20%, 30%, 40%, 50%, 60%, 70%, 75%, 80%, 85%, 90%, 95%, or 99% compared to a target protein level prior to modifying the cancer cells.

52. The method of any one of claims 1-51, wherein the compilation of therapeutic molecules comprises at least 1, at least 2, at least 3, at least 4, at least 5, at least 10, or at least 20 therapeutic agents of Table 3, Table 4, Table 10A, Table 10B, or Table 10C.

53. The method of any one of claims 1-52, wherein the selected therapeutic molecule comprises sorafenib, gilteritinib, quizartinib, glasdegib, ivosidenib, midostaurin, venetoclax, pemigatinib, or ruxolitinib.

54. The method of any one of claims 1-53, wherein the cancer cells prior to modification exhibit an active DNA damage response.

55. The method of any one of claims 1-54, wherein the cancer cells prior to modification exhibit a normal or a near-normal p53-dependent DNA double strand break (DSB) response.

56. The method of claim 55, wherein the normal or the near-normal p53-dependent DNA DSB response in the cancer cells prior to modification reflects an extent of p53-dependent DNA DSB response of the cancer of the subject.

57. The method of any one of claims 1-56, wherein the cancer in the subject comprises a hematologic malignancy.

58. The method of any one of claim 57, wherein the hematologic malignancy comprises a leukemia, a lymphoma, a myeloma, or a combination thereof.

59. The method of any one of claim 58, wherein the leukemia comprises Acute Myeloid Leukemia (AML).

60. The method of any one of claims 1-59, wherein the cancer in the subject comprises Glioblastoma Multiforme, Soft Tissue Tumors and Sarcomas, Kidney Adenomas and Adenocarcinomas, Liver Hepatocellular Carcinoma, Adrenocortical Carcinoma, Skin Cutaneous Melanoma, Mesothelioma, Prostate Adenocarcinomas, Diffuse Large B-cell Lymphoma, Cholangiocarcinoma, Cervical Squamous Cell Carcinoma and Endocervical Adenocarcinoma, Acute Myeloid Leukemia, Multiple Myeloma, Thymoma, Kidney Renal Clear Cell Carcinoma, Kidney Renal Papillary Cell Carcinoma, Acute Lymphoblastic Leukemia, Testicular Germ Cell Tumors, or Thyroid Carcinoma.

61. The method of any one of claims 1-60, wherein the cancer cells from the subject comprise primary cancer cells.

62. The method of any one of claims 1-61, wherein the cancer cells from the subject are isolated ex vivo and assayed for cancer cell viability or proliferation rate during in vitro propagation.

63. The method of any one of claims 1-62, wherein the cancer cells prior to modification do not comprise a mutation in p53.

64. The method of any one of claims 1-63, wherein the impairment of cancer cell viability or proliferation rate during in vitro propagation indicates a determination of a functional response of the cancer of the subject.

65. The method of claim 64, wherein the determination further comprises a functional call for suggested therapy.

66. The method of claim 65, wherein the functional call for suggested therapy indicates a weak, an intermediate, or a strong predicted response to the selected therapeutic molecule.

67. The method of claim 66, further comprising a weak predicted response to one or more small molecule therapeutics drugs listed in Table 3, 4 or 10, an intermediate predicted response to one or more small molecule therapeutics drugs listed in Table 3, 4 or 10, or a strong predicted response to one or more small molecule therapeutics drugs listed in Table 3, 4 or 10.

68. A method of treating cancer in a subject in need thereof, the method comprising administering to the subject a therapeutic molecule selected from a compilation of therapeutic molecules; wherein the cancer in the subject has been determined to be susceptible to the selected therapeutic molecule by a method comprising:
a) contacting a sample of cancer cells from the subject with a library of gene modulatory reagents to generate a plurality of modified cancer cells, wherein each modified cancer cell harbors one or more of the gene modulatory reagents, wherein the one or more of the gene modulatory reagents are capable of knocking down the function of a gene that encodes a protein target of a therapeutic molecule in the library of therapeutic molecules, wherein the one or more of the gene modulatory reagents does not cleave DNA, and
b) sequencing the plurality of modified cancer cells, wherein a gene modulatory reagent that impairs cell viability or proliferation rate will have fewer sequence reads than a gene modulatory reagent that does not impair cell viability of proliferation rate, wherein the one or more of the gene modulatory reagents capable of knocking down the function of the gene comprise a sequence complementarity to a gene that encodes for the protein targeted by the selected therapeutic molecule.

69. A method of perturbing gene function in a plurality of modified cancer cells from a subject having cancer, the method comprising delivering a library of gene modulatory reagents to a sample of cancer cells from the subject to generate the plurality of modified cancer cells; wherein each modified cancer cell harbors one or more of the gene modulatory reagents, and each gene modulatory reagent is capable of perturbing the function of a gene that encodes a protein target from a compilation of protein targets.

70. A compilation comprising a plurality of modified cancer cells, wherein each modified cancer cell harbors one or more gene modulatory reagents, and each gene modulatory reagent is capable of knocking down the function of a gene that encodes a protein target from a compilation of protein targets.

71. A method of evaluating the functional effect of perturbing gene activity in cancer cells from a subject, the method comprising: sequencing a plurality of modified cancer cells, wherein each modified cancer cell harbors one or more gene modulatory reagents, each gene perturbation reagent capable of knocking down the function of a gene that encodes a protein target in a library of protein targets; and wherein a gene perturbation reagent that impairs cell viability or proliferation rate will have fewer sequence reads than a gene perturbation reagent that does not impair cell viability or proliferation rate.

72. A library comprising a plurality of gene perturbation reagents, each gene perturbation reagent capable of knocking down the function of a gene that encodes a protein target from a library of protein targets.

73. The method of claim 6, wherein the catalytically inactive form of a CAS endonuclease is delivered to the cancer cells in a single vector or is delivered to the cancer cells in two of more vectors.

74. The method of any one of claims 8-12, wherein the catalytically inactive form of CAS endonuclease has one or more protein domains positioned in between the catalytically inactive form of CAS endonuclease and the epigenetic modulator.

75. A CRISPR interference (CRISPRi) system comprising a single vector transfer plasmid system comprising: a) a first promoter operably linked to a first nucleotide sequence encoding a catalytically inactive RNA-directed nuclease fused to a transcriptional repressor; and b) a second promoter operably linked to a second nucleotide sequence encoding a single-guide RNA (sgRNA) sequence comprising a CRISPR RNA (crRNA) sequence having complementarity to at least a portion of a gene that encodes a protein target and a trans-activating CRISPR RNA (tracrRNA) sequence.
